(19) United States
(12) Patent Application Publication
(10) Pub. No.: US 2025/0092014 A1
(43) Pub. Date: Mar. 20, 2025

(54) COMPOUNDS AND METHODS OF ACTIVATING LIPOPROTEIN LIPASE

(71) Applicant: Metrea Biosciences, Inc., San Francisco, CA (US)

(72) Inventors: Timothy Blizzard, Princeton, NJ (US); Paul Da Silva Jardine, Guilford, CT (US); Frederick Dewey, Winchester, MA (US); Nico Bräuer, Falkensee (DE); Giambattista Testolin, Potsdam (DE); Jelena Konstantinovic, Berlin (DE); Christian Lechner, Berlin (DE)

(21) Appl. No.: 18/261,942

(22) PCT Filed: Jan. 19, 2022
(86) PCT No.: PCT/US2022/012911

§ 371 Date: Jul. 18, 2023

Related U.S. Application Data
(60) Provisional application No. 63/139,096, filed on Jan. 19, 2021.
(60) Provisional application No. 63/139,098, filed on Jan. 19, 2021.

(51) Int. Cl. C07D 401/12; A61K 31/416; A61K 31/422; A61K 31/438; A61K 31/454; A61K 31/4545; A61K 31/496; A61K 31/5377; A61K 31/541; A61K 31/553; A61K 45/06; C07D 231/56; C07D 401/14; C07D 403/12; C07D 405/14; C07D 413/12; C07D 417/12; C07D 471/04

(52) U.S. Cl. CPC C07D 401/12; A61K 31/416; A61K 31/422; A61K 31/438; A61K 31/454; A61K 31/4545; A61K 31/496; A61K 31/5377; A61K 31/541; A61K 31/553; A61K 45/06; C07D 231/56; C07D 401/14; C07D 403/12; C07D 405/14; C07D 413/12; C07D 417/12; C07D 471/04

(57) ABSTRACT

The present disclosure is generally directed to activators of lipoprotein lipase useful in the treatment of diseases and disorders modulated by said enzyme and having the Formula (I),

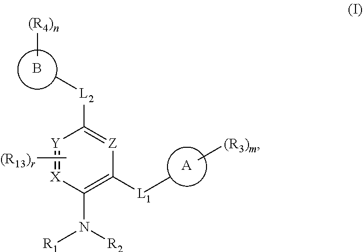

or a pharmaceutically acceptable salt, isomer, solvate, prodrug, or tautomer thereof.

### RELATED APPLICATION

This application claims the benefit of priority to U.S. Provisional Application No. 63/139,096, filed Jan. 19, 2021, and U.S. Provisional Application No. 63/139,098, filed Jan. 19, 2021, the contents of which are incorporated herein by reference in their entirety.

### FIELD

The present disclosure is directed to novel activators of lipoprotein lipase (LPL). The activators described herein can be useful in the treatment of dysmetabolism conditions. In particular, the disclosure is concerned with compounds and pharmaceutical compositions activating LPL, methods of treating dysmetabolism conditions associated with LPL including cardiovascular disease (CVD), insulin sensitization, and type 2 diabetes, and methods of synthesizing these compounds.

### BACKGROUND

Cardiovascular diseases (CVDs) are the major cause of mortality globally, resulting in an estimated 17.9 million deaths each year. CVDs include disorders of the heart and blood vessels, including coronary heart disease (CHD), cerebrovascular disease, rheumatic heart disease, among others. Elevated levels of blood triglycerides (TG), a type of lipid found in blood which provides energy, are considered a risk factor for the development of CVD. Even moderate increases in TG levels contribute to an increase in CVD risk, while severe elevations of TG levels are associated with an increased risk of pancreatitis. It is estimated that over 10 million patients in the United States alone have elevated TG levels and co-existing CHD.

Hypertriglyceridemia (HTG) is a common disorder in which triglyceride levels are elevated. HTG is usually seen with other metabolic abnormalities associated with increased CVD risk. An estimated 25% of Americans have HTG, which is defined as TG levels greater than 150 mg/dl, and are at increased CVD risk. In addition, greater that 3 million Americans have TGs greater that 500 mg/dl and are at even further risk for CHD and acute pancreatitis. Human genetic studies provide further support for the causal association between elevated TGs and CVD. Importantly, the strength of a variant's effect on TG levels is correlated with the magnitude of the variant's effect on CVD. (Sarwar et. al, Lancet 2010; 375(9726):1634-9). Genetically lowered TG levels are also associated with lower risk of recurrent CHD events independently of low-density lipoprotein cholesterol (LDL-C) levels. Miller et al. JACC 2008; 51:1724-30, Schwartz et. al. JACC 2015; 65(21) 2267-75. In a randomized controlled clinical trial, icosapent ethyl, a derivative of the omega-3 fatty acid eicosapentaenoic acid, reduced circulating TGs and the occurrence of a major adverse cardiovascular events by 25% in a study of >7000 patients who had elevated TGs but well controlled LDL-C.

As TGs circulate in the blood they are hydrolyzed by lipoprotein lipase (LPL). Despite the known association between TG levels and LPL activity, no efficacious TG lowering LPL therapies are currently available. Given the severity of CVD and the unmet clinical need, an effective therapeutic treatment is needed.

### SUMMARY

A first aspect of the present disclosure relates to compounds of Formula I:

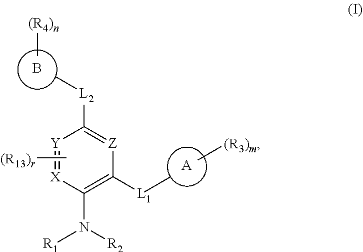

and pharmaceutically acceptable salts, isomers, solvates, prodrugs, or tautomers thereof,
   wherein:
X, Y, and Y are independently CH or N;
Ring A is heteroaryl;
Ring B is selected from aryl, and heteroaryl;
L₁ is selected from —N(R₇)—C(O)—N(R₇)—, —(CH₂)ₚ—N(R₇)—C(O)—, —C(O)—N(R₇)—(CH₂)ₚ—, —NR₇—S(O)₂—, and —S(O)₂—NR₇—;
L₂ is selected from —(CH₂)ₚ—N(R₇)—C(O)—, —C(O)—N(R₇)—(CH₂)ₚ—, —NH—CH₂—, —CH₂—NH—, —OCH₂—, —CH₂O—, —NR₇—S(O)₂—, —S(O)₂—NR₇—, —N(R₇)—NH₂—, or NH₂—N(R₇)—;
R₁ and R₂ are independently selected from H, C₁-C₆ alkyl, C₁-C₆ alkoxy, C₁-C₆ haloalkyl, C₂-C₆ alkenyl, and C₃-C₁₀ cycloalkyl, wherein the alkyl, alkoxy, or cycloalkyl is optionally substituted with one or more R₅; or
R₁ and R₂are taken together with the N atom to which they are attached to form 3- to 10-membered heterocyclyl, wherein the heterocyclyl is optionally substituted with one or more R₅;
each R₃ is independently selected from halogen, —CN, —OH, —C(═O)CH₃, C₁-C₆ alkyl, C₁-C₆ alkoxy, C₂-C₆ alkynyl, C₃-C₁₀ cycloalkyl, and C₆-C₁₀ aryl, wherein the alkyl, alkoxy, cycloalkyl, or aryl is optionally substituted with one or more R₆;
each R₄ is independently selected from halogen, —OH, —[C(R₁₀)₂]ₒ—COOH, —[C(R₁₀)₂]ₒ—CON(R₁₁)₂, C₁-C₆ alkyl, C₃-C₁₀ cycloalkyl, and 5- to 10-membered heteroaryl, wherein the alkyl and heteroaryl is optionally substituted with one or more groups selected from halogen, C₁-C₆ alkyl, —OH, and oxo;
each R₁₀ is independently halogen, C₁-C₆ alkyl, or C₃-C₁₀ carbocyclyl, or
two R₁₀ are taken together to form an C₃-C₆ cycloalkyl
each R₁ is independently hydrogen, C₁-C₆ alkyl, S(═O)₂R₁₂;
R₁₂ is C₁-C₆ alkyl or C₃-C₁₀ cycloalkyl;
each R₅ is independently selected from oxo, —OH, halogen, —C(═O)NH₂, —C(═O)OH, —CN, C₁-C₆ alkyl, C₁-C₆ alkoxy, C₁-C₆ haloalkyl, C₃-C₁₀ cycloalkyl, and aryl; wherein the alkyl, alkoxy, haloalkyl, cycloalkyl, and aryl is optionally substituted with one or more R₈; or
two R₅, together with the intervening atoms, forms a C₃-C₁₀ cycloalkyl or 3- to 10-membered heterocyclyl;
R₆ is independently selected from halogen, —OH, —NH₂, —CN, C₁-C₆ alkyl, C₂-C₆ alkynyl, C₁-C₆ alkoxy, C₁-C₆ haloalkoxy, C₁-C₆ haloalkyl, C₆-C₁₀ cycloalkyl, 3- to 10-membered heterocyclyl, heteroaryl, and C₆-C₁₀ aryl, wherein the cycloalkyl, heteroaryl or aryl is optionally substituted with one or more Rn;
R₇ is selected from H and C₁-C₃ alkyl; or
when L₂ is —N(R₇)—C(O)—, —C(O)—N(R₇)—, —N(R₇)—NH₂—, or —NH₂—N(R₇)—, and at least one R₄ is ortho to the L₂ substituent, R₄ and R₇, together with the atoms to which they are attached, can combine to form a 5- to 6-membered heterocyclyl or heteroaryl;
each R₈ is independently selected from —OH, —NH₂, and —C(═O)NH₂;
each R₉ is independently selected from halogen, —OH, —NH—, —CN, C₁-C₆ alkyl, C₁-C₆ alkoxy, C₁-C₆ haloalkoxy, C₁-C₆ haloalkyl, C₃-C₁₀ cycloalkyl, 3- to 10-membered heterocyclyl, heteroaryl, and C₆-C₁₀ aryl;
each R₁₃ is independently selected from halogen, —OH, —NH₂, —CN, C₁-C₆ alkyl, C₁-C₆ alkoxy, C₁-C₆ haloalkoxy, C₁-C₆ haloalkyl, C₃-C₁₀ cycloalkyl, 3- to 10-membered heterocyclyl, heteroaryl, and C₆-C₁₀ aryl;
m is an integer selected from 0, 1, 2, 3, 4, 5, and 6;
n is an integer selected from 0, 1, 2, 3, 4, and 5;
o is an integer selected from 0 to 2;
p is 0 or 1;
q is an integer selected from 0 to 3;
r is an integer selected from 0 to 3.

Another aspect of the present disclosure relates to compounds of Formula I:

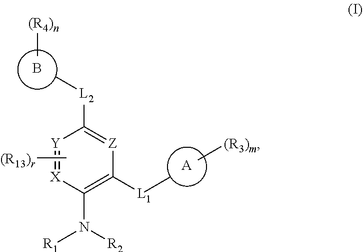

and pharmaceutically acceptable salts, isomers, solvates, prodrugs, or tautomers thereof,
   wherein:
X, Y, and Y are independently CH or N
Ring A is heteroaryl;
Ring B is selected from aryl, and heteroaryl;
L₁ is selected from —N(R₇)—C(O)—N(R₇)—, —(CH₂)ₚ—N(R₇)—C(O)—, —C(O)—N(R₇)—(CH₂)ₚ—, —NR₇—S(O)₂—, and —S(O)₂—NR₇—;
L₂ is selected from —(CH₂)ₚ—N(R₇)—C(O)—, —C(O)—N(R₇)—(CH₂)ₚ—, —NH—CH₂—, —CH₂—NH—, —OCH₂—, —CH₂O—, —NR₇—S(O)₂—, —S(O)₂—NR₇—, —N(R₇)—NH₂—, or NH₂—N(R₇)—;
R₁ and R₂are taken together with the N atom to which they are attached to form 3- to 10-membered heterocyclyl, wherein the heterocyclyl is optionally substituted with one or more R₅;
each R₁ is independently selected from halogen, —CN, —OH, —C(═O)CH₃, C₁-C₆ alkyl, C₁-C₆ alkoxy, C₂-C₆ alkynyl, C₃-C₁₀ cycloalkyl, and C₆-C₁₀ aryl, wherein the alkyl, alkoxy, cycloalkyl, or aryl is optionally substituted with one or more R₆;
each R₄ is independently selected from halogen, —OH, —[C(R₁₀)₂]ₒ—COOH, —[C(R₁₀)₂]ₒ—CON(R₁₁)₂, C₁-C₆ alkyl, C₃-C₁₀ cycloalkyl, and 5- to 10-membered heteroaryl, wherein the alkyl and heteroaryl is optionally substituted with one or more groups selected from halogen, C₁-C₆ alkyl, —OH, and oxo;
each R₁₀ is independently halogen, C₁-C₆ alkyl, or C₃-C₁₀ carbocyclyl, or
two R₁₀ are taken together to form an C₃-C₆ cycloalkyl each R₁₁ is independently hydrogen, C₁-C₆ alkyl, S(═O)₂R₂;
R₁₂ is C₁-C₆ alkyl or C₃-C₁ cycloalkyl;
each R₅ is independently selected from oxo, —OH, halogen, —C(═O)NH₂, —C(═O)OH, —CN, C₁-C₆ alkyl, C₁-C₆ alkoxy, C₁-C₆ haloalkyl, C₃-C₁₀ cycloalkyl, and aryl; wherein the alkyl, alkoxy, haloalkyl, cycloalkyl, and aryl is optionally substituted with one or more R₅; or
two R₅, together with the intervening atoms, forms a C₃-C₁₀ cycloalkyl or 3- to 10-membered heterocyclyl;
R₆ is independently selected from halogen, —OH, —NH₂, —CN, C₁-C₆ alkyl, C₂-C₆ alkynyl, C₁-C₆ alkoxy, C₁-C₆ haloalkoxy, C₁-C₆ haloalkyl, C₃-C₁₀ cycloalkyl, 3- to 10-membered heterocyclyl, heteroaryl, and C₆-C₁₀ aryl, wherein the cycloalkyl, heteroaryl or aryl is optionally substituted with one or more R₁₃;
R₇ is selected from H and C₁-C₃ alkyl; or
when L₂ is —N(R₇)—C(O)—, —C(O)—N(R₇)—, —N(R₇)—NH₂—, or —NH₂—N(R₇)—, and at least one R₄ is ortho to the L₂ substituent, R₄ and R₇, together with the atoms to which they are attached, can combine to form a 5- to 6-membered heterocyclyl or heteroaryl;
each R₈ is independently selected from —OH, —NH₂, and —C(═O)NH₂;
each R₁₃ is independently selected from halogen, —OH, —NH₂, —CN, C₁-C₆ alkyl, C₁-C₆ alkoxy, C₁-C₆ haloalkoxy, C₁-C₆ haloalkyl, C₆-C₁₀ cycloalkyl, 3- to 10-membered heterocyclyl, heteroaryl, and C₆-C₁₀ aryl;
m is an integer selected from 0, 1, 2, 3, 4, 5, and 6;
n is an integer selected from 0, 1, 2, 3, 4, and 5;
o is an integer selected from 0 to 2;
p is 0 or 1;
   r is an integer selected from 0 to 3.//.

Another aspect of the present disclosure is directed to pharmaceutical compositions comprising a compound of Formula (T), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the present disclosure relates to a method of treating a condition associated with activation of lipoprotein lipase (LPL). The method comprises administering to a patient in need of a treatment for conditions associated with activation of LPL an effective amount of a compound of Formula (I), or a pharmaceutically acceptable salt, hydrate, solvate, prodrug, stereoisomer, tautomer, or pharmaceutical composition thereof.

Another aspect of the present disclosure is directed to a method of treating or preventing a condition disclosed herein in a subject in need thereof. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present disclosure relates to compounds of Formula (I), and pharmaceutically acceptable salts, hydrates, solvates, prodrugs, stereoisomers, tautomers, or pharmaceutical compositions thereof, for use in the manufacture of a medicament for treating or preventing a condition disclosed herein.

Another aspect of the present disclosure relates to the use of a compound of Formula (I), or a pharmaceutically acceptable salt, hydrate, solvate, prodrug, stereoisomer, tautomer, or pharmaceutical composition thereof, in the treatment of a condition disclosed herein.

The present disclosure further provides methods of treating a condition associated with activation of LPL, including hypertriglyceridemia, moderate hypertriglyceridemia, severe hypertriglyceridemia, mixed dyslipidemia, familial chylomicronemia, type 1 hyperlipoproteinemia, type IIb hyperlipoproteinemia, type III hyperlipoproteinemia, type IV hyperlipoproteinemia, type V hyperlipoproteinemia, metabolic syndrome, partial lipodystrophy, prediabetes, type H diabetes, atherosclerotic cardiovascular disease, myocardial infarction, coronary heart disease, ischemic heart disease, peripheral vascular disease, cerebrovascular disease, peripheral arterial disease, acute pancreatitis, comprising administering to a patient suffering from at least one of said conditions a compound of Formula (I), or a pharmaceutically acceptable salt, hydrate, solvate, prodrug, stereoisomer, tautomer, or pharmaceutical composition thereof.

The present disclosure provides activation of LPL that are therapeutic agents in the treatment of diseases such as hypertriglyceridemia, moderate hypertriglyceridemia, severe hypertriglyceridemia, mixed dyslipidemia, familial chylomicronemia, type 1 hyperlipoproteinemia, type IIb hyperlipoproteinemia, type III hyperlipoproteinemia, type IV hyperlipoproteinemia, type V hyperlipoproteinemia, metabolic syndrome, partial lipodystrophy, prediabetes, type II diabetes, atherosclerotic cardiovascular disease, myocardial infarction, coronary heart disease, ischemic heart disease, peripheral vascular disease, cerebrovascular disease, peripheral arterial disease, acute pancreatitis.

The present disclosure further provides compounds and compositions with an improved efficacy and safety profile relative to known LPL activators. The present disclosure also provides agents with novel mechanisms of action toward LPL in the treatment of various types of diseases including hypertriglyceridemia, moderate hypertriglyceridemia, severe hypertriglyceridemia, mixed dyslipidemia, familial chylomicronemia, type 1 hyperlipoproteinemia, type IIb hyperlipoproteinemia, type III hyperlipoproteinemia, type IV hyperlipoproteinemia, type V hyperlipoproteinemia, metabolic syndrome, partial lipodystrophy, prediabetes, type II diabetes, atherosclerotic cardiovascular disease, myocardial infarction, coronary heart disease, ischemic heart disease, peripheral vascular disease, cerebrovascular disease, peripheral arterial disease, acute pancreatitis.

In some aspects, the present disclosure provides an intermediate as described herein, being suitable for use in a method for preparing a compound as described herein (e.g., the intermediate is selected from the intermediates described in Examples 1-13).

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed present disclosure. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE FIGURES

FIG. 1 is a chart demonstrating the protective action of Compound 158 on LPL from inactivation by ANGPTLA4 in a Chinese hamster ovary (CHO) pgsA-745/GPIHBP1 LPL lipolysis assay.

FIG. 2 is a chart demonstrating the intrinsic activation of LPL by Compound 158 on in a Chinese hamster ovary (CHO) pgsA-745/GPIHBP1 LPL lipolysis assay.

FIG. 3 is a chart illustrating Compound 171 blunts hypertriglyceridemia response in a dose-dependent fashion in an oral fat tolerance test.

FIG. 4 is an AUC (area under the curve) analysis of Compound 171 dose response in the oral fat tolerance test using zero (O) as baseline.

FIG. 5 is a chart illustrating Compound 158 blunts hypertriglyceridemia response in a dose-dependent fashion in an oral fat tolerance test.

FIG. 6 is an AUC analysis of Compound 158 dose response in the oral fat tolerance test using zero (O) as baseline.

FIG. 7 is a chart illustrating Compound 172 blunts hypertriglyceridemia response in a dose-dependent fashion in an oral fat tolerance test.

FIG. 8 is an AUC analysis of Compound 172 dose response in the oral fat tolerance test using initial value as baseline.

FIG. 9 is a chart illustrating Compound 170 blunts hypertriglyceridemia response in a dose-dependent fashion in an oral fat tolerance test.

FIG. 10A is a chart illustrating Compound 173 blunts hypertriglyceridemia response in a dose-dependent fashion in an oral fat tolerance test.

FIG. 10B is a chart illustrating Compound 174 blunts hypertriglyceridemia response in a dose-dependent fashion in an oral fat tolerance test.

FIG. 10C is a chart illustrating Compound 175 blunts hypertriglyceridemia response in a dose-dependent fashion in an oral fat tolerance test.

FIG. 10D is a chart illustrating Compound 176 blunts hypertriglyceridemia response in a dose-dependent fashion in an oral fat tolerance test.

### DETAILED DESCRIPTION

The present disclosure relates to compounds and compositions that are capable of activating the activity of LPL.

In a first aspect of the present disclosure, the compounds of Formula (I) are described:

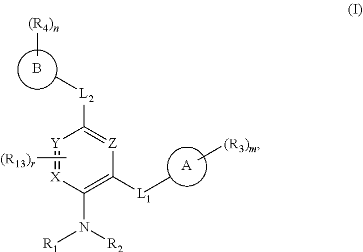

and pharmaceutically acceptable salts, hydrates, solvates, prodrugs, stereoisomers, and tautomers thereof, wherein Ring A, Ring B, X, Y, Z, L₁, L₂, R₁, R₂, R₃, R₁, R₅, m, n, and r are described herein.

The details of the present disclosure are set forth in the accompanying description below. Although methods and materials similar or equivalent to those described herein can be used in the practice or testing of the present disclosure, illustrative methods and materials are now described.

Other features, objects, and advantages of the present disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this present disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OH, —OP(O)(OH)₂, —OC(OXC₁-C₆) alkyl, —C(OXC₁-C₆) alkyl, —OC(O)O(C₁-C₆) alkyl, —NH—, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆) alkyl, and S(O)N((C₁-C₆) alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted. Furthermore, when containing two fused rings, the aryl groups herein defined may have a saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or polycyclic aromatic radical of 5 to 24 ring atoms, containing one or more ring heteroatoms selected from N, O, S, P, Se, or B, the remaining ring atoms being C. Heteroaryl as herein defined also means a bicyclic heteroaromatic group wherein the heteroatom is selected from N, O, S, P, Se, or B. Heteroaryl as herein defined also means a tricyclic heteroaromatic group containing one or more ring heteroatoms selected from N, O, S, P, Se, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolinyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1V′-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzoxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, 1H-tetrazolyl, tetrazolyl, 1,2,3-triazolyl, and derivatives thereof. Furthermore, when containing two or more fused rings, the heteroaryl groups defined herein may have one or more saturated or partially unsaturated ring fused with a fully unsaturated aromatic ring, e.g., a 5-membered heteroaromatic ring containing 1 to 3 heteroatoms selected from N, O, S, P, Se, or B, or a 6-membered heteroaromatic ring containing 1 to 3 nitrogens, wherein the saturated or partially unsaturated ring includes 0 to 4 heteroatoms selected from N, O, S, P, Se, or B, and is optionally substituted with one or more oxo. In heteroaryl ring systems containing more than two fused rings, a saturated or partially unsaturated ring may further be fused with a saturated or partially unsaturated ring described herein. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, or benzo[c][1,2]oxaborol-1(3H)-olyl.

“Halogen” or “halo” refers to fluorine, chlorine, bromine, or iodine.

“Alkyl” refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, t-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above-mentioned monovalent alkyl groups may be an alkylene by abstraction of a second hydrogen atom from the alkyl. As herein defined, alkylene may also be a C₁-C₆alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH)₂—, —CH₂C(CH₃)₂—, —CH₂CH₂CH₂—, —CH₂CH₂CH₂CH₂—, and the like.

“Cycloalkyl” means a saturated or partially unsaturated hydrocarbon monocyclic or polycyclic (e.g., fused, bridged, or spiro rings) system having 3 to 30 carbon atoms (e.g., C₁-C₁₂, C₃-C₁₀, or C₃-C₆). Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, bicyclo[2.2.2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a,4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, adamantyl, and derivatives thereof. In the case of polycyclic cycloalkyl, only one of the rings in the cycloalkyl needs to be non-aromatic.

“Heterocyclyl”, “heterocycle” or “heterocycloalkyl” refers to a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms (such as O, N, S, P, Se, or B), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted one or more halogen. Examples of haloalkoxy groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C═N.

The term “amine” as used herein refers to primary (R—NH₂, R w H), secondary ((R)₂—NH, R₂≠H) and tertiary ((R)₃—N, R≠H) amines. A substituted amine is intended to mean an amine where at least one of the hydrogen atoms has been replaced by the substituent.

The term “amino” as used herein means a substituent containing at least one nitrogen atom. Specifically, —NH₂, —NH(alkyl) or alkylamino, —N(alkyl)₂ or dialkylamino, amide-, carbamide-, urea, and sulfamide substituents are included in the term “amino”.

The term “solvate” refers to a complex of variable stoichiometry formed by a solute and solvent. Such solvents for the purpose of the present disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The present disclosure also contemplates isotopically-labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” when used in connection with a compound is an amount effective for treating or preventing a condition in a subject as described herein.

The term “carrier” as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug” as used in this disclosure, means a compound which is convertible in vivo by metabolic means (e.g., by hydrolysis) to a disclosed compound.

The present disclosure relates to compounds or pharmaceutically acceptable salts, hydrates, solvates, prodrugs, stereoisomers, or tautomers thereof, capable of activating LPL, which are useful for the treatment of diseases and disorders associated with activation of LPL. The present disclosure further relates to compounds, or pharmaceutically acceptable salts, hydrates, solvates, prodrugs, stereoisomers, or tautomers thereof, which can be useful for activating LPL.

In some embodiments, the compounds of Formula (I) have the structure of Formula (Ia):

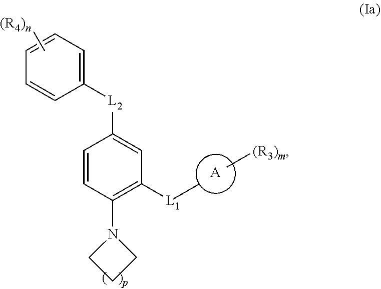

or a pharmaceutically acceptable salt, isomer, solvate, prodrug, or tautomer thereof, wherein p is an integer selected from 0, 1, 2, 3, 4, 5, 6, and 7.

In some embodiments, p is 0. In some embodiments, p is 1. In some embodiments, p is 2. In some embodiments, p is 3. In some embodiments, p is 4. In some embodiments, p is 5. In some embodiments, p is 6. In some embodiments, p is 7.

In some embodiments, the compounds of Formula (I) have the structure of Formula (Ia-1):

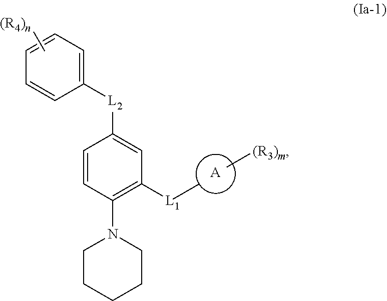

or a pharmaceutically acceptable salt, isomer, solvate, prodrug, or tautomer thereof.

In some embodiments, the compounds of Formula (I) have the structure of Formulae (Ib-1), (Ib-2), (Ib-3), (Ib-4), or (Ib-5):

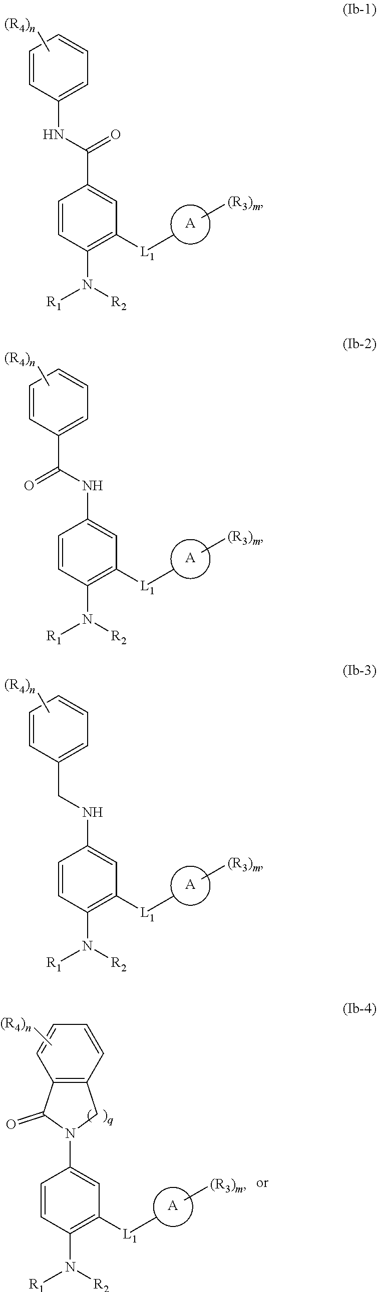

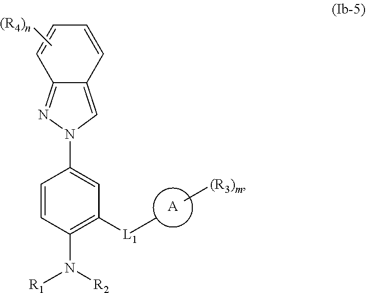

or a pharmaceutically acceptable salt, isomer, solvate, prodrug, or tautomer thereof,
wherein q is an integer selected from 1 and 2.

In some embodiments, q is 1. In some embodiments, q is 2.

In some embodiments, the compounds of Formula (I) have the structure of Formulae (Ic-1) or (Ic-2):

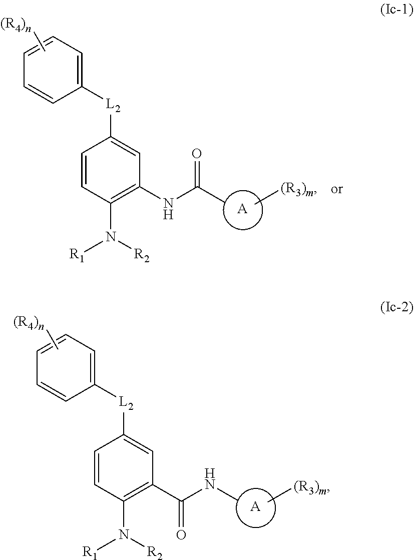

or a pharmaceutically acceptable salt, isomer, solvate, prodrug, or tautomer thereof.

In some embodiments, the compounds of Formula (I) have the structure of Formula (Id-1), (Id-2), (Id-3), (Id-4), (Id-5), (Id-6), or (Id-7):

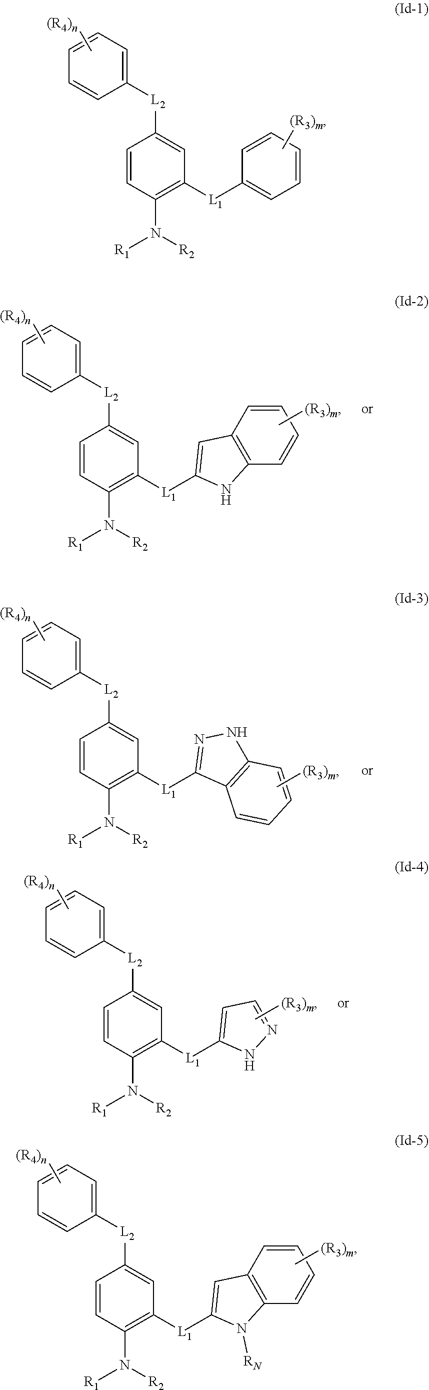

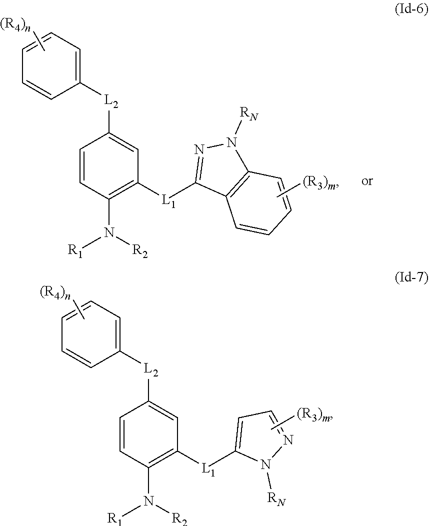

or a pharmaceutically acceptable salt, isomer, solvate, prodrug, or tautomer thereof;

wherein R_{N} is selected from C₁-C₆ alkyl, wherein the alkyl is optionally substituted with one or more halo or aryl.

In some embodiments, the compounds of Formula (I) have the structure of Formulae (Ie-1) or (Ie-2):

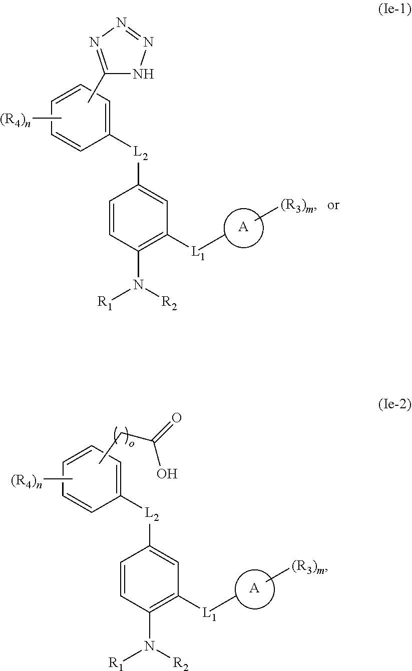

or a pharmaceutically acceptable salt, isomer, solvate, prodrug, or tautomer thereof.

In some embodiments, X is CH or N. In other embodiments, X is CH. In other embodiments, X is N. In some embodiments X is —CR₁₃. In some embodiments, Y is CH or N. In other embodiments, Y is CH. In other embodiments, Y is N. In some embodiments Y is —CR₁₃. In some embodiments, Z is CH or N. In other embodiments, Z is CH. In other embodiments, Z is N. In some embodiments Z is —CR₁₃.

In some embodiments, Ring A is heteroaryl. In some embodiments, Ring A is monocyclic.

In some embodiments, Ring A is bicyclic.

In some embodiments, Ring B is selected from aryl or heteroaryl. In some embodiments, Ring B is monocyclic. In some embodiments, Ring B is bicyclic.

In some embodiments, Ring A is selected from

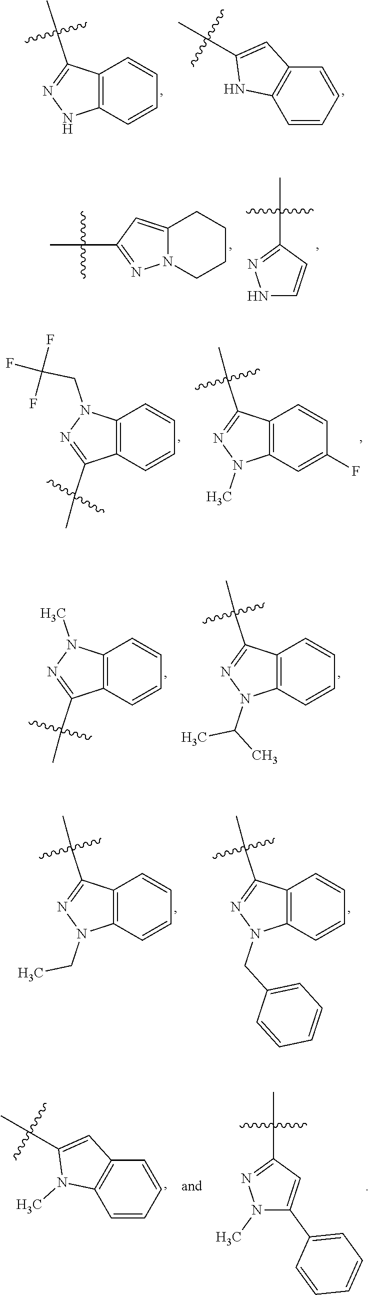

In some embodiments, L₁ is selected from —N(R₇)—C(O)—N(R₇)—, —(CH₂)ₚ—N(R₇)—C(O)—, —C(O)—N(R₇)(CH₂)ₚ—, —NR₇—S(O)₂—, and —S(O)₂—NR₇—. In some embodiments, L₁ is —N(R₇)—C(O)—N(R)—. In some embodiments, L₁ is —(CH₂)ₚ—N(R₇)—C(O)—. In some embodiments, L₁ is —C(O)—N(R₇)—(CH₂)ₚ—. In some embodiments, L₁ is —NR₇—S(O)₂—. In some embodiments, L₁-S(O)₂—NR₇—. In some embodiments, L₁ is —NH—C(O)—. In some embodiments, L₁ is —C(O)—NH—

In some embodiments, L₂ is selected from —(CH₂)ₚ—N(R₇)—C(O)—, —C(O)—N(R₇)—(CH₂)ₚ—, —NR₇—CH₂—, —CH₂—NR₇—, —OCH₂—, —CH₂O—, —NR₇—S(O)₂—, —S(O)₂—NR₇—, —N(R₇)—NH₂—, or NH₂—N(R₇)—. In some embodiments, L₂ is —(CH₂)ₚ—N(R₇)—C(O)—. In some embodiments, L₂ is —C(O)—N(R₇HCH₂)ₚ—. In some embodiments, L₂ is —NR₇—CH₂—. In some embodiments, L₂ is —CH₂—NR₇—. In some embodiments, L₂ is —OCH₂—. In some embodiments, L₂ is —CH₂O—. In some embodiments, L₂ is —NR₇—S(O)₂—. In some embodiments, L₂ is —S(O)₂—NR₇—. In some embodiments, L₂ is —N(R₇)—NH₂—. In some embodiments, L₂ is —NH₂—N(R₇)—. In some embodiments, L₂ is —NH—C(O)—. In some embodiments, L₂ is —C(O)—NH—. In some embodiments, L₂ is —N(R₇)—C(O)—. In some embodiments, L₂ is —C(O)—N(R₇)—. In some embodiments, L₂ is —NH—CH₂—. In some embodiments, L₂ is —CH₂—NH—. In some embodiments, L₂ is —NH—S(O)₂—. In some embodiments, L₂ is —S(O)₂—NH—.

In some embodiments, R₁ is H. In some embodiments, R₁ is C₁-C₆ alkyl. In some embodiments, R₁ is C₁-C₆ alkyl substituted with one or more R₅. In some embodiments, R₁ is C₁-C₆ alkoxy. In some embodiments, R₁ is C₁-C₆ alkoxy substituted with one or more R₅. In some embodiments, R₁ is C₁-C₆ haloalkyl. In some embodiments, R₁ is C₂-C₆ alkenyl. In some embodiments, R₁ is C₃-C₁₀ cycloalkyl. In some embodiments, R₁ is C₃-C₁₀ cycloalkyl substituted with one or more R₅. In some embodiments, R₁ is C₅ cycloalkyl.

In some embodiments, R₁ is

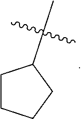 [SMILES: *C1CCCC1]

In some embodiments, R₂ is H. In some embodiments, R₂ is C₁-C₆ alkyl. In some embodiments, R₂ is C₁-C₆ alkyl substituted with one or more 11. In some embodiments, R₂ is C₁-C₆ alkoxy. In some embodiments, R₂ is C₁-C₆ alkoxy substituted with one or more R₅. In some embodiments, R₂ is C₁-C₆ haloalkyl. In some embodiments, R₂ is C₂-C₆ alkenyl. In some embodiments, R₂ is C₃-C₁₀ cycloalkyl. In some embodiments, R₂ is C₃-C₁₀ cycloalkyl substituted with one or more R₅. In some embodiments, R₂ is C₅ cycloalkyl.

In some embodiments, R₂ is

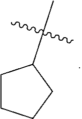 [SMILES: *C1CCCC1]

In some embodiments, R₁ is C₃-C₁₀ cycloalkyl and R₂ is H.

In some embodiments, R₁ and R₂ are taken together with the N atom to which they are attached to form 3- to 10-membered heterocyclyl. In some embodiments, R₁ and R₂ are taken together with the N atom to which they are attached to form 3- to 10-membered heterocyclyl optionally substituted with one or more R₅. In some embodiments, R₁ and R₂, taken together with the N atom to which they are attached, form 6-membered heterocyclyl. In some embodiments, R₁ and R₂, taken together with the N atom to which they are attached, form piperidinyl.

In some embodiments, each R₃ is independently selected from halogen, —CN, —OH, —C(O)CH₃, C₁-C₆ alkyl, C₁-C₆ alkoxy, C₂-C₆ alkynyl, C₃-C₁₀ cycloalkyl, and C₆-C₁₀ aryl. In some embodiments, R₃ is halogen. In some embodiments, R₃ is fluoro. In some embodiments, R₃ is chloro. In some embodiments, R₃ is bromo. In some embodiments, R₃ is iodo. In some embodiments, R₃ is —CN. In some embodiments, R₃ is —OH. In some embodiments, R₃ is —C(O)CH₃. In some embodiments, R₃ is C₁-C₆ alkyl. In some embodiments, R₃ is C₁-C₆ alkyl substituted with one or more R₆. In some embodiments, R₃ is methyl. In some embodiments, R₃ is ethyl. In some embodiments, R₃ is propyl. In some embodiments, R₃ is butyl. In some embodiments, R₃ is pentyl. In some embodiments, R₃ is hexyl. In some embodiments, R₃ is C₁-C₆ alkoxy. In some embodiments, R₃ is C₁-C₆ alkoxy substituted with one or more R₅. In some embodiments, R₃ is C₂-C₆ alkynyl. In some embodiments, R₃ is C₂-C₆ alkynyl substituted with one or more R₆. In some embodiments, R₃ is C₃-C₁₀ cycloalkyl. In some embodiments, R₃ is C₃-C₁₀ cycloalkyl substituted with one or more R₆. In some embodiments, R₃ is C₆-C₁₀ aryl. In some embodiments, R₃ is C₆-C₁₀ aryl substituted with one or more R₆.

In some embodiments, each R₃ is independently selected from —F, —Cl, —CH₃,

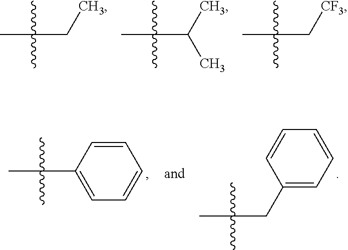

In some embodiments, each R₄ is independently selected from halogen, —OH, —[C(R₁₀)₂]ₒ—COOH, —[C(R₁₀)₂]ₒ—CON(R₁₁)₂, C₁-C₆ alkyl, C₃-C₁₀ cycloalkyl, and 5- to 10-membered heteroaryl, wherein the alkyl, cycloalkyl, and the heteroaryl is optionally substituted with one or more groups selected from halogen, C₁-C₆ alkyl, —OH, and oxo. In some embodiments, R₄ is halogen. In some embodiments, R₄ is fluoro. In some embodiments, R₄ is chloro. In some embodiments, R₄ is bromo. In some embodiments, R₄ is iodo. In some embodiments, R₄ is —OH. In some embodiments, R₄ is —[C(R₁₀)₂]ₒ—COOH. In some embodiments, R₄ is —CH₂—COOH. In some embodiments, R₄ is —COOH. In some embodiments, R₄ is —[C(R₁₀)₂]ₒ—CON(R₁₁)₂. In some embodiments, R₄ is —(CH₂)ₒ-C(O)NHS(O)₂R₁₂. In some embodiments, Ra is —CH₂—C(O)NHS(O)₂R₁₂. In some embodiments, R₄ is —C(O)NHS(O)₂R₁₂. In some embodiments, R₄ is 5- to 10-membered heteroaryl. In some embodiments, R₄ is monocyclic heteroaryl. In some embodiments, R₄ is 5-membered heteroaryl. In some embodiments, R₄ is 5-membered heteroaryl containing at least one N atom. In some embodiments, R₄ is 5-membered heteroaryl containing four N atoms. In some embodiments, R₄ is 5- to 10-membered heteroaryl containing at least one N atom. In some embodiments, R₄ is 5- to 10-membered heteroaryl containing four N atoms. In some embodiments, R₄ is tetrazole.

In some embodiments, each R is independently selected from —CH₂—COOH, —COOH, —CH₂—C(O)NHS(O)₂CH₃, —F,

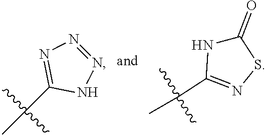 [SMILES: *c1nnn[nH]1 ; *c1nsc(=O)[nH]1]

In some embodiments, R₄ is

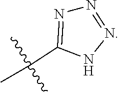 [SMILES: *c1nnn[nH]1]

In some embodiments, each R₅ is independently selected from oxo, —OH, halogen, —C(═O)NH₂, —C(═O)OH, —CN, C₁-C₆ alkyl, C₁-C₆ alkoxy, C₁-C₆ haloalkyl, C₃-C₁₀ cycloalkyl, and aryl. In some embodiments, the alkyl, alkoxy, haloalkyl, cycloalkyl, and aryl is optionally substituted with one or more R₈. In some embodiments, R₅ is oxo. In some embodiments, R₅ is —OH. In some embodiments, R₅ is halogen. In some embodiments, R₅ is fluro. In some embodiments, R₅ is chloro. In some embodiments, R₅ is bromo. In some embodiments, R₅ is iodo. In some embodiments, R₅ is —C(═O)NH₂. In some embodiments, R₅ is —C(═O)OH. In some embodiments, R₅ is —CN. In some embodiments, R₅ is C₁-C₆ alkyl. In some embodiments, R₅ is C₁-C₆ alkoxy. In some embodiments, R₅ is C₁-C₆ haloalkyl. In some embodiments, R₅ is C₃-C₁₀ cycloalkyl. In some embodiments, R₅ is aryl. In some embodiments, the alkyl, alkoxy, haloalkyl, cycloalkyl, and aryl is optionally substituted with one or more R₅.

In some embodiments, two R₅, together with the intervening atoms, forms a C₃-C₁₀ cycloalkyl or 3- to 10-membered heterocyclyl.

In some embodiments, R₆ is independently selected from halogen, —OH, —NH₂, —CN, C₁-C₆ alkyl, C₂-C₆ alkynyl, C₁-C₆ alkoxy, C₁-C₆ haloalkoxy, C₁-C₆ haloalkyl, C₃-C₁₀ cycloalkyl, 3to 10-membered heterocyclyl, heteroaryl, and C₆-C₁₀ aryl. In some embodiments, the cycloalkyl, heteroaryl or aryl is optionally substituted with one or more R₁₃. In some embodiments, R₆ is halogen. In some embodiments, R₆ is fluro. In some embodiments, R₆ is chloro. In some embodiments, R₆ is bromo. In some embodiments, R₆ is iodo. In some embodiments, R₆ is —OH. In some embodiments, R₆ is —NH₂. In some embodiments, R₆ is —CN. In some embodiments, R₆ is C₁-C₆ alkyl. In some embodiments, R₆ is C₂-C₆ alkynyl. In some embodiments, R₆ is C₁-C₆alkoxy. In some embodiments, Rr, is C₁-C₆ haloalkyl. In some embodiments, R₆, is C₆-C₁₀ cycloalkyl. In some embodiments, R₆ is 3- to 10-membered heterocyclyl. In some embodiments, R₆, is heteroaryl. In some embodiments, R₆ is C₆-C₁₀ aryl. In some embodiments, R₆ is C₆-C₁₀ aryl substituted with one or more halogen or —CH₂— cycloalkyl.

In some embodiments, R₇ is H. In some embodiments, R₇ is C₁-C₃ alkyl. In some embodiments, R₇ is methyl. In some embodiments, R₇ is ethyl. In some embodiments, R₇ is n-propyl. In some embodiments, R₇ is isopropyl.

In some embodiments, each R₅ is independently selected from —OH, —NH₂, and —C(═O)NH₂.

In some embodiments, R₅ is —OH. In some embodiments, Ra is —NH₂. In some embodiments, R₅ is —C(═O)NH₂.

In some embodiments, each R₁₀ is independently halogen, C₁-C₆ alkyl, or C₃-C₁₀ carbocyclyl. In some embodiments, R₁₀ is halogen. In some embodiments, R₁₀ is C₁-C₆ alkyl. In some embodiments, R₁₀ is C₃-C₁₀ carbocyclyl.

In some embodiments, two R₁₀ are taken together to form an C₃-C₆ cycloalkyl.

In some embodiments, each R₁₁ is independently hydrogen, C₁-C₆ alkyl, or —S(═O)₂R₁₂. In some embodiments, R₁₁ is hydrogen. In some embodiments, R₁₁ is C₁-C₆ alkyl. In some embodiments, R₁₁ is —S(═O)₂R₁₂.

In some embodiments, R₁₂ is C₁-C₆ alkyl or C₃-C₁₀ cycloalkyl. In some embodiments, R₁₂ is C₁-C₆ alkyl. In some embodiments, R₁₂ is C₆-C₁₀ cycloalkyl.

In some embodiments, each R₁₃ is independently selected from halogen, —OH, —NH₂, —CN, C₁-C₆ alkyl, C₁-C₆ alkoxy, C₁-C₆ haloalkoxy, C₁-C₆ haloalkyl, C₃-C₁₀ cycloalkyl, 3- to 10-membered heterocyclyl, heteroaryl, and C₆-C₁₀ aryl. In some embodiments, each R₁. is independently selected from halogen, —OH, —NH₂, and —CN. In some embodiments, each Rn is independently selected from C₁-C₆ alkyl, C₁-C₆ alkoxy, C₁-C₆ haloalkoxy, C₁-C₆ haloalkyl, C₁-C₁₀ cycloalkyl, 3- to 10-membered heterocyclyl, heteroaryl, and C₆-C₁₀ aryl.

In some embodiments, L₂ is —S(O)₂—NR₇— and an R₄ is ortho to the L₂ substituent, Ra and R₇ combine to form a 5- to 6-membered heterocyclyl. In some embodiments, L₂ is —NR₇—S(O)₂- and an R₄ is ortho to the L₂ substituent, R₄ and R—combine to form a 5- to 6-membered heterocyclyl. In some embodiments, L₂ is —(CH₂)ₚ—N(R₇)—C(O)—and an R₄ is ortho to the L₂ substituent, R₄ and R₇ combine to form a 5- to 6-membered heterocyclyl. In some embodiments, L₂ is —C(O)—N(R)—(CH₂)ₚ—and an R₄ is ortho to the L₂ substituent, R₄ and R₇ combine to form a 5- to 6-membered heterocyclyl. In some embodiments, L₂ is —N(R₇)—C(O)—and an R₄ is ortho to the L₂ substituent, R₄ and R—combine to form a 5- to 6-membered heterocyclyl. In some embodiments, L₂ is —C(O)—N(R₇)—and an R₄ is ortho to the L₂ substituent, R₄ and R₇ combine to form a 5- to 6-membered heterocyclyl. In some embodiments, L₂ is —N(R₇)—C(O)—and an R₄ is ortho to the L₂ substituent, R₄ and R—combine to form a 5-membered heterocyclyl. In some embodiments, L₂ is —C(O)—N(R₇)—and an R₄ is ortho to the L₂ substituent, R₄ and R₇ combine to form a 6-membered heterocyclyl. In some embodiments, L₂ is —N(R₇)—NH₂—and an R₄ is ortho to the L₂ substituent, R₄ and R₇ combine to form a 5- to 6-membered heterocyclyl. In some embodiments, L₂ is —N(R₇)—NH₂—and an R₄ is ortho to the L₂ substituent, R₄ and R₇ combine to form a 5- to 6-membered heterocyclyl. In some embodiments, L₂ is —NH₂—N(R₇)—and an R₄ is ortho to the L₂ substituent, R₄ and R₇ combine to form a 5-membered heterocyclyl. In some embodiments, L₂ is —NH₂—N(R₇)—and an R₄ is ortho to the L₂ substituent, R₄ and R₇ combine to form a 6-membered heterocyclyl.

In some embodiments, m is an integer selected from 0, 1, 2, 3, 4, 5, and 6. In some embodiments, m is 0. In some embodiments, m is 1. In some embodiments, m is 2. In some embodiments, m is 3. In some embodiments, m is 4. In some embodiments, m is 5. In some embodiments, m is 6.

In some embodiments, n is an integer selected from 0, 1, 2, 3, 4, and 5. In some embodiments, n is 0. In some embodiments, n is 1. In some embodiments, n is 2. In some embodiments, n is 3. In some embodiments, n is 4. In some embodiments, n is 5.

In some embodiments, o is an integer selected from 0 to 2. In some embodiments, o is 0.

In some embodiments, o is 1. In some embodiments, o is 2.

In some embodiments, p is 0 or 1. In some embodiments, p is 0. In some embodiments, p is 1.

In some embodiments, r is an integer selected from 0 to 3. In some embodiments, r is 0. In some embodiments, r is 1. In some embodiments, r is 2. In some embodiments, r is 3.

In some embodiments, suitable compounds of the instant disclosure are:

| Compound # | Name |
|---|---|
| 1 | 2-(5-fluoro-2-(N-methyl-4-(piperidin-1-yl)-3-(1-(2,2,2-trifluoroethyl)-1H- |
| | indazole-3-carboxamido)benzamido)phenyl)acetic acid |
| 2 | 4-(4-(piperidin-1-yl)-3-(1-(2,2,2-trifluoroethyl)-1H-indazole-3- |
| | carboxamido)benzamido)nicotinic acid |
| 3 | 5-(2-(4-(piperidin-1-yl)-3-(1-(2,2,2-trifluoroethyl)-1H-indazole-3- |
| | carboxamido)benzamido)-2-fluorophenyl)acetic acid |
| 4 | 2-(2-(4-(piperidin-1-yl)-3-(1-(2,2,2-trifluoroethyl)-1H-indazole-3- |
| | carboxamido)benzamido)phenyl)acetic acid |
| 5 | 2-(4-(piperidin-1-yl)-3-(1-(2,2,2-trifluoroethyl)-1H-indazole-3- |
| | carboxamido)benzamido)benzoic acid |
| 6 | 2-(5-fluoro-2-(4-(piperidin-1-yl)-3-(1-(2,2,2-trifluoroethyl)-1H-indazole-3- |
| | carboxamido)benzamido)phenyl)propanoic acid |
| 7 | 2-fluoro-2-(5-fluoro-2-(4-(piperidin-1-yl)-3-(1-(2,2,2-trifluoroethyl)-1H- |
| | indazole-3-carboxamido)benzamido)phenyl)acetic acid |
| 7a | 2,2-difluoro-2-(5-fluoro-2-(4-(piperidin-1-yl)-3-(1-(2,2,2-trifluoroethyl)-1H- |
| | indazole-3-carboxamido)benzamido)phenyl)acetic acid |
| 8 | 2,2-difluoro-2-(2-(4-(piperidin-1-yl)-3-(1-(2,2,2-trifluoroethyl)-1H-indazole-3- |
| | carboxamido)benzamido)phenyl)acetic acid |
| 9 | 3-(2-(4-(piperidin-1-yl)-3-(1-(2,2,2-trifluoroethyl)-1H-indazole-3- |
| | carboxamido)benzamido)phenyl)propanoic acid |
| 10 | 3-(4-(piperidin-1-yl)-3-(1-(2,2,2-trifluoroethyl)-1H-indazole-3- |
| | carboxamido)benzamido)benzoic acid |
| 11 | 4-(4-(piperidin-1-yl)-3-(1-(2,2,2-trifluoroethyl)-1H-indazole-3- |
| | carboxamido)benzamido)benzoic acid |
| 11a | 2-(4-(4-(piperidin-1-yl)-3-(1-(2,2,2-trifluoroethyl)-1H-indazole-3- |
| | carboxamido)benzamido)phenyl)acetic acid |
| 12 | 2-(3-fluoro-4-(4-(piperidin-1-yl)-3-(1-(2,2,2-trifluoroethyl)-1H-indazole-3- |
| | carboxamido)benzamido)phenyl)acetic acid |
| 13 | N-(5-((4-fluoro-2-(2-(methylsulfonamido)-2-oxoethyl)phenyl)carbamoyl)-2- |
| | (piperidin-1-yl)phenyl)-1-(2,2,2-trifluoroethyl)-1H-indazole-3-carboxamide |
| 14 | N-(5-((2-(2-(ethylsulfonamido)-2-oxoethyl)-4-fluorophenyl)carbamoyl)-2- |
| | (piperidin-1-yl)phenyl)-1-(2,2,2-trifluoroethyl)-1H-indazole-3-carboxamide |
| 15 | N-(5-((2-(2-(cyclopropanesulfonamido)-2-oxoethyl)-4-fluorophenyl)carbamoyl)- |
| | 2-(piperidin-1-yl)phenyl)-1-(2,2,2-trifluoroethyl)-1H-indazole-3-carboxamide |
| 16 | N-(5-((2-(2-amino-2-oxoethyl)phenyl)carbamoyl)-2-(piperidin-1-yl)phenyl)-1- |
| | (2,2,2-trifluoroethyl)-1H-indazole-3-carboxamide |
| 16a | 2-(2-(4-(piperidin-1-yl)-3-(1-(2,2,2-trifluoroethyl)-1H-indazole-3- |
| | carboxamido)benzamido)pyridin-3-yl)acetic acid |
| 17 | 2-(4-(piperidin-1-yl)-3-(1-(2,2,2-trifluoroethyl)-1H-indazole-3- |
| | carboxamido)benzamido)nicotinic acid |
| 18 | 2-((4-(piperidin-1-yl)-3-(1-(2,2,2-trifluoroethyl)-1H-indazole-3- |
| | carboxamido)benzamido)methyl)benzoic acid |
| 19 | 3-((4-(piperidin-1-yl)-3-(1-(2,2,2-trifluoroethyl)-1H-indazole-3- |
| | carboxamido)benzamido)methyl)benzoic acid |
| 20 | 2-(5-fluoro-2-((4-(piperidin-1-yl)-3-(1-(2,2,2-trifluoroethyl)-1H-indazole-3- |
| | carboxamido)phenyl)sulfonamido)phenyl)acetic acid |
| 21 | 2-(5-fluoro-2-((N-methyl-4-(piperidin-1-yl)-3-(1-(2,2,2-trifluoroethyl)-1H- |
| | indazole-3-carboxamido)phenyl)sulfonamido)phenyl)acetic acid |
| 22 | 2-(2-(4-(piperidin-1-yl)-3-(1-(2,2,2-trifluoroethyl)-1H-indazole-3- |
| | carboxamido)benzamido)-5-(trifluoromethyl)phenyl)acetic acid |
| 23 | 2-(5-chloro-2-(4-(piperidin-1-yl)-3-(1-(2,2,2-trifluoroethyl)-1H-indazole-3- |
| | carboxamido)benzamido)phenyl)acetic acid |
| 24 | 2-(3-fluoro-2-(4-(piperidin-1-yl)-3-(1-(2,2,2-trifluoroethyl)-1H-indazole-3- |
| | carboxamido)benzamido)phenyl)acetic acid |
| 25 | 2-(3-chloro-2-(4-(piperidin-1-yl)-3-(1-(2,2,2-trifluoroethyl)-1H-indazole-3- |
| | carboxamido)benzamido)phenyl)acetic acid |
| 26 | 3-chloro-2-(4-(piperidin-1-yl)-3-(1-(2,2,2-trifluoroethyl)-1H-indazole-3- |
| | carboxamido)benzamido)benzoic acid |
| 27 | 3-fluoro-2-(4-(piperidin-1-yl)-3-(1-(2,2,2-trifluoroethyl)-1H-indazole-3- |
| | carboxamido)benzamido)benzoic acid |
| 28 | 2-(5-fluoro-2-(4-(piperidin-1-yl)-3-(1-(2,2,2-trifluoroethyl)-1H-indazole-3- |
| | carboxamido)benzamido)phenyl)butanoic acid |
| 29 | 2-cyclopropyl-2-(5-fluoro-2-(4-(piperidin-1-yl)-3-(1-(2,2,2-trifluoroethyl)-1H- |
| | indazole-3-carboxamido)benzamido)phenyl)acetic acid |
| 30 | 2-(5-fluoro-2-(4-(piperidin-1-yl)-3-(1-(2,2,2-trifluoroethyl)-1H-indazole-3- |
| | carboxamido)benzamido)phenyl)-2-methylpropanoic acid |
| 31 | 1-(5-fluoro-2-(4-(piperidin-1-yl)-3-(1-(2,2,2-trifluoroethyl)-1H-indazole-3- |
| | carboxamido)benzamido)phenyl)cyclopropane-1-carboxylic acid |
| 32 | 2-(2-methyl-6-(4-(piperidin-1-yl)-3-(1-(2,2,2-trifluoroethyl)-1H-indazole-3- |
| | carboxamido)benzamido)phenyl)acetic acid |
| 33 | 2-(5-methyl-2-(4-(piperidin-1-yl)-3-(1-(2,2,2-trifluoroethyl)-1H-indazole-3- |
| | carboxamido)benzamido)phenyl)acetic acid |
| 34 | 2-(4-methyl-2-(4-(piperidin-1-yl)-3-(1-(2,2,2-trifluoroethyl)-1H-indazole-3- |
| | carboxamido)benzamido)phenyl)acetic acid |
| 35 | 2-(3-methyl-2-(4-(piperidin-1-yl)-3-(1-(2,2,2-trifluoroethyl)-1H-indazole-3- |
| | carboxamido)benzamido)phenyl)acetic acid |
| 36 | 2-(5-cyclopropyl-2-(4-(piperidin-1-yl)-3-(1-(2,2,2-trifluoroethyl)-1H-indazole-3- |
| | carboxamido)benzamido)phenyl)acetic acid |
| 37 | 2-(2-fluoro-6-(4-(piperidin-1-yl)-3-(1-(2,2,2-trifluoroethyl)-1H-indazole-3- |
| | carboxamido)benzamido)phenyl)acetic acid |
| 38 | 2-(4-fluoro-2-(4-(piperidin-1-yl)-3-(1-(2,2,2-trifluoroethyl)-1H-indazole-3- |
| | carboxamido)benzamido)phenyl)acetic acid |
| 39 | 2-(2-(4-(azetidin-1-yl)-3-(1-(2,2,2-trifluoroethyl)-1H-indazole-3- |
| | carboxamido)benzamido)-5-fluorophenyl)acetic acid |
| 40 | 2-(2-(4-(4,4-difluoropiperidin-1-yl)-3-(1-(2,2,2-trifluoroethyl)-1H-indazole-3- |
| | carboxamido)benzamido)-5-fluorophenyl)acetic acid |
| 41 | 2-(5-fluoro-2-(4-(piperidin-1-yl)-3-((1-(2,2,2-trifluoroethyl)-1H-indazol-3- |
| | yl)carbamoyl)benzamido)phenyl)acetic acid |
| 42 | 2-(5-fluoro-2-(2-methyl-4-(piperidin-1-yl)-3-(1-(2,2,2-trifluoroethyl)-1H- |
| | indazole-3-carboxamido)benzamido)phenyl)acetic acid |
| 43 | 2-(5-fluoro-2-(5-(piperidin-1-yl)-4-(1-(2,2,2-trifluoroethyl)-1H-indazole-3- |
| | carboxamido)picolinamido)phenyl)acetic acid |
| 44 | 2-(5-fluoro-2-(6-(piperidin-1-yl)-5-(1-(2,2,2-trifluoroethyl)-1H-indazole-3- |
| | carboxamido)nicotinamido)phenyl)acetic acid |
| 45 | 2-(5-fluoro-2-(5-(piperidin-1-yl)-6-(1-(2,2,2-trifluoroethyl)-1H-indazole-3- |
| | carboxamido)picolinamido)phenyl)acetic acid |
| 46 | 1-(4-((2-(carboxymethyl)-4-fluorophenyl)carbamoyl)-2-(1-(2,2,2-trifluoroethyl)- |
| | 1H-indazole-3-carboxamido)phenyl)piperidine-4-carboxylic acid |
| 47 | 2-(5-fluoro-2-(4-(piperazin-1-yl)-3-(1-(2,2,2-trifluoroethyl)-1H-indazole-3- |
| | carboxamido)benzamido)phenyl)acetic acid |
| 48 | 2-(2-(4-(4-cyanopiperidin-1-yl)-3-(1-(2,2,2-trifluoroethyl)-1H-indazole-3- |
| | carboxamido)benzamido)-5-fluorophenyl)acetic acid |
| 49 | 2-(2-(4-(2-azaspiro[3.3]heptan-2-yl)-3-(1-(2,2,2-trifluoroethyl)-1H-indazole-3- |
| | carboxamido)benzamido)-5-fluorophenyl)acetic acid |
| 50 | 2-(5-fluoro-2-(4-(pyrrolidin-1-yl)-3-(1-(2,2,2-trifluoroethyl)-1H-indazole-3- |
| | carboxamido)benzamido)phenyl)acetic acid |
| 50a | 2-(5-fluoro-2-((4-(piperidin-1-yl)-3-((1-(2,2,2-trifluoroethyl)-1H-indazol-3- |
| | yl)carbamoyl)phenyl)carbamoyl)phenyl)acetic acid |
| 51 | 2-(2-((4-(piperidin-1-yl)-3-((1-(2,2,2-trifluoroethyl)-1H-indazol-3- |
| | yl)carbamoyl)phenyl)carbamoyl)phenyl)acetic acid |
| 52 | 2-(2-(4-(3-azabicyclo[3.1.0]hexan-3-yl)-3-(1-(2,2,2-trifluoroethyl)-1H-indazole- |
| | 3-carboxamido)benzamido)-5-fluorophenyl)acetic acid |
| 53 | 2-(2-(4-(5-azaspiro[2.4]heptan-5-yl)-3-(1-(2,2,2-trifluoroethyl)-1H-indazole-3- |
| | carboxamido)benzamido)-5-fluorophenyl)acetic acid |
| 54 | 2-(2-(4-((1s,4s)-7-azabicyclo[2.2.1]heptan-7-yl)-3-(1-(2,2,2-trifluoroethyl)-1H- |
| | indazole-3-carboxamido)benzamido)-5-fluorophenyl)acetic acid |
| 55 | 2-(5-fluoro-2-(2-methyl-4-(piperidin-1-yl)-5-(1-(2,2,2-trifluoroethyl)-1H- |
| | indazole-3-carboxamido)benzamido)phenyl)acetic acid |
| 56 | 2-(5-fluoro-2-(4-morpholino-3-(1-(2,2,2-trifluoroethyl)-1H-indazole-3- |
| | carboxamido)benzamido)phenyl)acetic acid |
| 57 | 2-(5-fluoro-2-(4-(4-methylpiperazin-1-yl)-3-(1-(2,2,2-trifluoroethyl)-1H- |
| | indazole-3-carboxamido)benzamido)phenyl)acetic acid |
| 58 | rac2-(2-(4-(2-azabicyclo[2.2.1]heptan-2-yl)-3-(1-(2,2,2-trifluoroethyl)-1H- |
| | indazole-3-carboxamido)benzamido)-5-fluorophenyl)acetic acid |
| 59 | 2-(5-fluoro-2-(4-(2-oxopiperidin-1-yl)-3-(1-(2,2,2-trifluoroethyl)-1H-indazole-3- |
| | carboxamido)benzamido)phenyl)acetic acid |
| 60 | 2-(5-fluoro-2-(4-(2-oxooxazolidin-3-yl)-3-(1-(2,2,2-trifluoroethyl)-1H-indazole- |
| | 3-carboxamido)benzamido)phenyl)acetic acid |
| 61 | 2-(2-(4-(4,4-dimethylpiperidin-1-yl)-3-(1-(2,2,2-trifluoroethyl)-1H-indazole-3- |
| | carboxamido)benzamido)-5-fluorophenyl)acetic acid |
| 62 | 2-(2-(4-(3,3-difluoropiperidin-1-yl)-3-(1-(2,2,2-trifluoroethyl)-1H-indazole-3- |
| | carboxamido)benzamido)-5-fluorophenyl)acetic acid |
| 63 | 2-(2-(4-(1,4-oxazepan-4-yl)-3-(1-(2,2,2-trifluoroethyl)-1H-indazole-3- |
| | carboxamido)benzamido)-5-fluorophenyl)acetic acid |
| 64 | 2-(5-fluoro-2-(4-(4-methylpiperidin-1-yl)-3-(1-(2,2,2-trifluoroethyl)-1H- |
| | indazole-3-carboxamido)benzamido)phenyl)acetic acid |
| 65 | 2-(5-fluoro-2-(4-(4-methoxypiperidin-1-yl)-3-(1-(2,2,2-trifluoroethyl)-1H- |
| | indazole-3-carboxamido)benzamido)phenyl)acetic acid |
| 66 | 2-(2-(4-(1,1-dioxidothiomorpholino)-3-(1-(2,2,2-trifluoroethyl)-1H-indazole-3- |
| | carboxamido)benzamido)-5-fluorophenyl)acetic acid |
| 67 | 2-(5-fluoro-2-(4-(2-methylpiperidin-1-yl)-3-(1-(2,2,2-trifluoroethyl)-1H- |
| | indazole-3-carboxamido)benzamido)phenyl)acetic acid |
| 68 | 2-(2-(4-(2,6-dimethylpiperidin-1-yl)-3-(1-(2,2,2-trifluoroethyl)-1H-indazole-3- |
| | carboxamido)benzamido)-5-fluorophenyl)acetic acid |
| 69 | 2-(5-fluoro-2-(4-(2-(hydroxymethyl)piperidin-1-yl)-3-(1-(2,2,2-trifluoroethyl)- |
| | 1H-indazole-3-carboxamido)benzamido)phenyl)acetic acid |
| 70 | 2-(2-(4-(2-(aminomethyl)piperidin-1-yl)-3-(1-(2,2,2-trifluoroethyl)-1H-indazole- |
| | 3-carboxamido)benzamido)-5-fluorophenyl)acetic acid |
| 71 | 2-(2-(4-(2-carbamoylpiperidin-1-yl)-3-(1-(2,2,2-trifluoroethyl)-1H-indazole-3- |
| | carboxamido)benzamido)-5-fluorophenyl)acetic acid |
| 72 | 2-(2-(4-(2-ethylpiperidin-1-yl)-3-(1-(2,2,2-trifluoroethyl)-1H-indazole-3- |
| | carboxamido)benzamido)-5-fluorophenyl)acetic acid |
| 73 | 2-(5-fluoro-2-(4-(3-methylpiperidin-1-yl)-3-(1-(2,2,2-trifluoroethyl)-1H- |
| | indazole-3-carboxamido)benzamido)phenyl)acetic acid |
| 74 | 2-(2-(4-(3,5-dimethylpiperidin-1-yl)-3-(1-(2,2,2-trifluoroethyl)-1H-indazole-3- |
| | carboxamido)benzamido)-5-fluorophenyl)acetic acid |
| 75 | 2-(5-fluoro-2-(4-(4-hydroxypiperidin-1-yl)-3-(1-(2,2,2-trifluoroethyl)-1H- |
| | indazole-3-carboxamido)benzamido)phenyl)acetic acid |
| 76 | 2-(2-(4-(2-oxa-6-azaspiro[3.3]heptan-6-yl)-3-(1-(2,2,2-trifluoroethyl)-1H- |
| | indazole-3-carboxamido)benzamido)-5-fluorophenyl)acetic acid |
| 77 | (R)-2-(5-fluoro-2-(4-(2-methylpiperidin-1-yl)-3-(1-(2,2,2-trifluoroethyl)-1H- |
| | indazole-3-carboxamido)benzamido)phenyl)acetic acid |
| 78 | (S)-2-(5-fluoro-2-(4-(2-methylpiperidin-1-yl)-3-(1-(2,2,2-trifluoroethyl)-1H- |
| | indazole-3-carboxamido)benzamido)phenyl)acetic acid |
| 79 | (R)-2-(5-fluoro-2-(4-(3-methylpiperidin-1-yl)-3-(1-(2,2,2-trifluoroethyl)-1H- |
| | indazole-3-carboxamido)benzamido)phenyl)acetic acid |
| 80 | (S)-2-(5-fluoro-2-(4-(3-methylpiperidin-1-yl)-3-(1-(2,2,2-trifluoroethyl)-1H- |
| | indazole-3-carboxamido)benzamido)phenyl)acetic acid |
| 81 | 2-(2-(4-(2-(difluoromethyl)piperidin-1-yl)-3-(1-(2,2,2-trifluoroethyl)-1H- |
| | indazole-3-carboxamido)benzamido)-5-fluorophenyl)acetic acid |
| 82 | 2-(2-(4-(3-(difluoromethyl)piperidin-1-yl)-3-(1-(2,2,2-trifluoroethyl)-1H- |
| | indazole-3-carboxamido)benzamido)-5-fluorophenyl)acetic acid |
| 83 | 2-(2-(4-(4-(difluoromethyl)piperidin-1-yl)-3-(1-(2,2,2-trifluoroethyl)-1H- |
| | indazole-3-carboxamido)benzamido)-5-fluorophenyl)acetic acid |
| 84 | 2-(5-fluoro-2-(4-(3-methoxypiperidin-1-yl)-3-(1-(2,2,2-trifluoroethyl)-1H- |
| | indazole-3-carboxamido)benzamido)phenyl)acetic acid |
| 85 | 2-(5-fluoro-2-(4-(3-hydroxypiperidin-1-yl)-3-(1-(2,2,2-trifluoroethyl)-1H- |
| | indazole-3-carboxamido)benzamido)phenyl)acetic acid |
| 86 | 2-(5-fluoro-2-(4-(3-(hydroxymethyl)piperidin-1-yl)-3-(1-(2,2,2-trifluoroethyl)- |
| | 1H-indazole-3-carboxamido)benzamido)phenyl)acetic acid |
| 87 | 2-(2-(4-(3-cyanopiperidin-1-yl)-3-(1-(2,2,2-trifluoroethyl)-1H-indazole-3- |
| | carboxamido)benzamido)-5-fluorophenyl)acetic acid |
| 88 | 2-(2-(4-(3-carbamoylpiperidin-1-yl)-3-(1-(2,2,2-trifluoroethyl)-1H-indazole-3- |
| | carboxamido)benzamido)-5-fluorophenyl)acetic acid |
| 89 | 2-(2-(4-(3-ethylpiperidin-1-yl)-3-(1-(2,2,2-trifluoroethyl)-1H-indazole-3- |
| | carboxamido)benzamido)-5-fluorophenyl)acetic acid |
| 90 | 2-(2-(4-(4-ethylpiperidin-1-yl)-3-(1-(2,2,2-trifluoroethyl)-1H-indazole-3- |
| | carboxamido)benzamido)-5-fluorophenyl)acetic acid |
| 91 | 2-(2-(4-(4-azaspiro[2.5]octan-4-yl)-3-(1-(2,2,2-trifluoroethyl)-1H-indazole-3- |
| | carboxamido)benzamido)-5-fluorophenyl)acetic acid |
| 92 | 2-(2-(4-(5-azaspiro[2.5]octan-5-yl)-3-(1-(2,2,2-trifluoroethyl)-1H-indazole-3- |
| | carboxamido)benzamido)-5-fluorophenyl)acetic acid |
| 93 | 2-(2-(4-(6-azaspiro[2.5]octan-6-yl)-3-(1-(2,2,2-trifluoroethyl)-1H-indazole-3- |
| | carboxamido)benzamido)-5-fluorophenyl)acetic acid |
| 94 | 2-(2-(3-(1-(cyclopropylmethyl)-1H-indazole-3-carboxamido)-4-(piperidin-1- |
| | yl)benzamido)-5-fluorophenyl)acetic acid |
| 95 | 2-(5-fluoro-2-(3-(1-isobutyl-1H-indazole-3-carboxamido)-4-(piperidin-1- |
| | yl)benzamido)phenyl)acetic acid |
| 96 | 2-(2-(3-(1-(2,2-difluoroethyl)-1H-indazole-3-carboxamido)-4-(piperidin-1- |
| | yl)benzamido)-5-fluorophenyl)acetic acid |
| 97 | 2-(2-(3-(1-cyclopropyl-1H-indazole-3-carboxamido)-4-(piperidin-1- |
| | yl)benzamido)-5-fluorophenyl)acetic acid |
| 98 | 2-(2-(3-(1-(cyclobutylmethyl)-1H-indazole-3-carboxamido)-4-(piperidin-1- |
| | yl)benzamido)-5-fluorophenyl)acetic acid |
| 99 | 2-(5-fluoro-2-(3-(1-(2-hydroxyethyl)-1H-indazole-3-carboxamido)-4-(piperidin- |
| | 1-yl)benzamido)phenyl)acetic acid |
| 100 | 2-(2-(3-(1-(2-aminoethyl)-1H-indazole-3-carboxamido)-4-(piperidin-1- |
| | yl)benzamido)-5-fluorophenyl)acetic acid |
| 101 | 2-(5-fluoro-2-(4-(piperidin-1-yl)-3-(1-(2-(pyrrolidin-1-yl)ethyl)-1H-indazole-3- |
| | carboxamido)benzamido)phenyl)acetic acid |
| 102 | 2-(2-(3-(1-(cyanomethyl)-1H-indazole-3-carboxamido)-4-(piperidin-1- |
| | yl)benzamido)-5-fluorophenyl)acetic acid |
| 103 | 2-(5-fluoro-2-(4-(piperidin-1-yl)-3-(1-(piperidin-4-ylmethyl)-1H-indazole-3- |
| | carboxamido)benzamido)phenyl)acetic acid |
| 104 | 2-(5-fluoro-2-(4-(piperidin-1-yl)-3-(1-((tetrahydro-2H-pyran-4-yl)methyl)-1H- |
| | indazole-3-carboxamido)benzamido)phenyl)acetic acid |
| 105 | 2-(5-fluoro-2-(3-(1-(2-methoxyethyl)-1H-indazole-3-carboxamido)-4-(piperidin- |
| | 1-yl)benzamido)phenyl)acetic acid |
| 106 | 2-(5-fluoro-2-(3-(1-(2-hydroxy-2-methylpropyl)-1H-indazole-3-carboxamido)-4- |
| | (piperidin-1-yl)benzamido)phenyl)acetic acid |
| 107 | 2-(5-fluoro-2-(4-(piperidin-1-yl)-3-(1-(2,2,2-trifluoroethyl)-1H-pyrazolo[4,3- |
| | c]pyridine-3-carboxamido)benzamido)phenyl)acetic acid |
| 108 | 2-(5-fluoro-2-(3-(imidazo[1,2-a]pyridine-3-carboxamido)-4-(piperidin-1- |
| | yl)benzamido)phenyl)acetic acid |
| 109 | 2-(5-fluoro-2-(3-(imidazo[1,2-a]pyridine-2-carboxamido)-4-(piperidin-1- |
| | yl)benzamido)phenyl)acetic acid |
| 110 | 2-(5-fluoro-2-(3-(1-methylimidazo[1,5-a]pyridine-3-carboxamido)-4-(piperidin- |
| | 1-yl)benzamido)phenyl)acetic acid |
| 111 | 2-(5-fluoro-2-(4-(piperidin-1-yl)-3-(1-(2,2,2-trifluoroethyl)-1H-pyrazolo[4,3- |
| | b]pyridine-3-carboxamido)benzamido)phenyl)acetic acid |
| 112 | 2-(5-fluoro-2-(4-(piperidin-1-yl)-3-(1-(2,2,2-trifluoroethyl)-1H-pyrazolo[3,4- |
| | c]pyridine-3-carboxamido)benzamido)phenyl)acetic acid |
| 113 | 2-(5-fluoro-2-(4-(piperidin-1-yl)-3-(1-(2,2,2-trifluoroethyl)-1H-pyrazolo[3,4- |
| | b]pyridine-3-carboxamido)benzamido)phenyl)acetic acid |
| 114 | 2-(5-fluoro-2-(4-(piperidin-1-yl)-3-((1-(2,2,2-trifluoroethyl)-1H-indazole)-3- |
| | sulfonamido)benzamido)phenyl)acetic acid |
| 115 | 2-(5-fluoro-2-(3-(4-phenyl-1-(2,2,2-trifluoroethyl)-1H-pyrazole-3-carboxamido)- |
| | 4-(piperidin-1-yl)benzamido)phenyl)acetic acid |
| 116 | 2-(5-fluoro-2-(3-(5-phenyl-1-(2,2,2-trifluoroethyl)-1H-pyrazole-3-carboxamido)- |
| | 4-(piperidin-1-yl)benzamido)phenyl)acetic acid |
| 117 | 2-(2-(3-(4-cyclopropyl-1-(2,2,2-trifluoroethyl)-1H-pyrazole-3-carboxamido)-4- |
| | (piperidin-1-yl)benzamido)-5-fluorophenyl)acetic acid |
| 118 | 2-(5-fluoro-2-(4-(piperidin-1-yl)-3-(1-((tetrahydrofuran-3-yl)methyl)-1H- |
| | indazole-3-carboxamido)benzamido)phenyl)acetic acid |
| 119 | 2-(5-fluoro-2-(4-(piperidin-1-yl)-3-(1-((tetrahydrofuran-2-yl)methyl)-1H- |
| | indazole-3-carboxamido)benzamido)phenyl)acetic acid |
| 120 | 2-(5-fluoro-2-(3-(1-(2-morpholinoethyl)-1H-indazole-3-carboxamido)-4- |
| | (piperidin-1-yl)benzamido)phenyl)acetic acid |
| 121 | 2-(5-fluoro-2-(4-(piperidin-1-yl)-3-(1-(3,3,3-trifluoropropyl)-1H-indazole-3- |
| | carboxamido)benzamido)phenyl)acetic acid |
| 122 | 2-(5-fluoro-2-(4-(piperidin-1-yl)-3-(1-(prop-2-yn-1-yl)-1H-indazole-3- |
| | carboxamido)benzamido)phenyl)acetic acid |
| 123 | 2-(2-(3-(1-(but-2-yn-1-yl)-1H-indazole-3-carboxamido)-4-(piperidin-1- |
| | yl)benzamido)-5-fluorophenyl)acetic acid |
| 124 | 2-(5-fluoro-2-(3-(1-((5-methylisoxazol-3-yl)methyl)-1H-indazole-3- |
| | carboxamido)-4-(piperidin-1-yl)benzamido)phenyl)acetic acid |
| 125 | 2-(5-fluoro-2-(3-(1-((5-methyl-1,2,4-oxadiazol-3-yl)methyl)-1H-indazole-3- |
| | carboxamido)-4-(piperidin-1-yl)benzamido)phenyl)acetic acid |
| 126 | 2-(5-fluoro-2-(3-(1-((5-methyl-1,3,4-oxadiazol-2-yl)methyl)-1H-indazole-3- |
| | carboxamido)-4-(piperidin-1-yl)benzamido)phenyl)acetic acid |
| 127 | 2-(2-(3-(1-((1-cyanocyclopropyl)methyl)-1H-indazole-3-carboxamido)-4- |
| | (piperidin-1-yl)benzamido)-5-fluorophenyl)acetic acid |
| 128 | 2-(5-fluoro-2-(4-(piperidin-1-yl)-3-(1-(2,2,2-trifluoroethyl)-1H-indole-3- |
| | carboxamido)benzamido)phenyl)acetic acid |
| 129 | 2-(5-fluoro-2-(4-(piperidin-1-yl)-3-(3-(1-(2,2,2-trifluoroethyl)-1H-indazol-3- |
| | yl)ureido)benzamido)phenyl)acetic acid |
| 130 | 2-(5-fluoro-2-(3-(2-methyl-1-(2,2,2-trifluoroethyl)-1H-indole-3-carboxamido)-4- |
| | (piperidin-1-yl)benzamido)phenyl)acetic acid |
| 131 | 2-(5-fluoro-2-(4-(piperidin-1-yl)-3-(1-(2-(trifluoromethoxy)ethyl)-1H-indazole- |
| | 3-carboxamido)benzamido)phenyl)acetic acid |
| 132 | 2-(2-(3-(1-((1,4-dioxan-2-yl)methyl)-1H-indazole-3-carboxamido)-4-(piperidin- |
| | 1-yl)benzamido)-5-fluorophenyl)acetic acid |
| 133 | 2-(5-fluoro-2-(4-(piperidin-1-yl)-3-(1-((tetrahydro-2H-pyran-3-yl)methyl)-1H- |
| | indazole-3-carboxamido)benzamido)phenyl)acetic acid |
| 134 | 2-(5-fluoro-2-(3-(3-phenyl-1-(2,2,2-trifluoroethyl)-1H-pyrazole-5-carboxamido)- |
| | 4-(piperidin-1-yl)benzamido)phenyl)acetic acid |
| 135 | 2-(5-fluoro-2-(4-(piperidin-1-yl)-3-(2-(2,2,2-trifluoroethyl)-2H-indazole-3- |
| | carboxamido)benzamido)phenyl)acetic acidhydrochloride |
| 136 | (S)-2-(5-fluoro-2-(4-(piperidin-1-yl)-3-(1-((tetrahydrofuran-2-yl)methyl)-1H- |
| | indazole-3-carboxamido)benzamido)phenyl)acetic acid |
| 137 | (R)-2-(5-fluoro-2-(4-(piperidin-1-yl)-3-(1-((tetrahydrofuran-2-yl)methyl)-1H- |
| | indazole-3-carboxamido)benzamido)phenyl)acetic acid |
| 138 | 2-(5-fluoro-2-(3-(5-fluoro-1-(2,2,2-trifluoroethyl)-1H-indazole-3-carboxamido)- |
| | 4-(piperidin-1-yl)benzamido)phenyl)acetic acid |
| 139 | 2-(5-chloro-2-(3-(5-fluoro-1-(2,2,2-trifluoroethyl)-1H-indazole-3-carboxamido)- |
| | 4-(piperidin-1-yl)benzamido)phenyl)acetic acid |
| 140 | 2-(5-fluoro-2-(3-(5-methoxy-1-(2,2,2-trifluoroethyl)-1H-indazole-3- |
| | carboxamido)-4-(piperidin-1-yl)benzamido)phenyl)acetic acid |
| 141 | 2-(5-fluoro-2-(3-(4-methyl-1-(2,2,2-trifluoroethyl)-1H-indazole-3-carboxamido)- |
| | 4-(piperidin-1-yl)benzamido)phenyl)acetic acid |
| 142 | 2-(5-fluoro-2-(3-(5-methyl-1-(2,2,2-trifluoroethyl)-1H-indazole-3-carboxamido)- |
| | 4-(piperidin-1-yl)benzamido)phenyl)acetic acid |
| 143 | 2-(5-fluoro-2-(3-(6-methyl-1-(2,2,2-trifluoroethyl)-1H-indazole-3-carboxamido)- |
| | 4-(piperidin-1-yl)benzamido)phenyl)acetic acid |
| 144 | 2-(5-fluoro-2-(3-(7-methyl-1-(2,2,2-trifluoroethyl)-1H-indazole-3-carboxamido)- |
| | 4-(piperidin-1-yl)benzamido)phenyl)acetic acid |
| 145 | 2-(5-fluoro-2-(4-(piperidin-1-yl)-3-(1-(2,2,2-trifluoroethyl)-1,4,5,6- |
| | tetrahydrocyclopenta[c]pyrazole-3-carboxamido)benzamido)phenyl)acetic acid |
| 146 | 2-(5-fluoro-2-(4-(piperidin-1-yl)-3-(1-(1,1,1-trifluoropropan-2-yl)-1H-indazole- |
| | 3-carboxamido)benzamido)phenyl)acetic acid |
| 147 | 2-(2-(3-(1-(1-cyclopropylethyl)-1H-indazole-3-carboxamido)-4-(piperidin-1- |
| | yl)benzamido)-5-fluorophenyl)acetic acid |
| 148 | 2-(5-fluoro-2-(4-(piperidin-1-yl)-3-(1-(1-(tetrahydro-2H-pyran-4-yl)ethyl)-1H- |
| | indazole-3-carboxamido)benzamido)phenyl)acetic acid |
| 149 | 2-(2-(3-((1-(cyanomethyl)-1H-indazol-3-yl)carbamoyl)-4-(piperidin-1- |
| | yl)benzamido)-5-fluorophenyl)acetic acid |
| 150 | 2-(5-fluoro-2-(4-(piperidin-1-yl)-3-((1-((tetrahydro-2H-pyran-4-yl)methyl)-1H- |
| | indazol-3-yl)carbamoyl)benzamido)phenyl)acetic acid |
| 151 | 2-(5-fluoro-2-(4-(piperidin-1-yl)-3-((1-(2,2,2-trifluoroethyl)-4,5,6,7-tetrahydro- |
| | 1H-indazol-3-yl)carbamoyl)benzamido)phenyl)acetic acid |
| 152 | (R)-2-(2-(4-(2-ethylpiperidin-1-yl)-3-(1-(2,2,2-trifluoroethyl)-1H-indazole-3- |
| | carboxamido)benzamido)-5-fluorophenyl)acetic acid |
| 153 | (S)-2-(2-(4-(2-ethylpiperidin-1-yl)-3-(1-(2,2,2-trifluoroethyl)-1H-indazole-3- |
| | carboxamido)benzamido)-5-fluorophenyl)acetic acid |
| 154 | 2-(5-fluoro-2-(4-(piperidin-1-yl)-3-(1-(2,2,2-trifluoroethyl)-4,5,6,7-tetrahydro- |
| | 1H-indazole-3-carboxamido)benzamido)phenyl)acetic acid |
| 155 | 2-(2-(3-(5,5-difluoro-5,6-dihydro-4H-pyrrolo[1,2-b]pyrazole-2-carboxamido)-4- |
| | (piperidin-1-yl)benzamido)-5-fluorophenyl)acetic acid |
| 156 | 2-(5-fluoro-2-(4-(piperidin-1-yl)-3-(1-(2,2,2-trifluoroethyl)-1,4,6,7- |
| | tetrahydropyrano[4,3-c]pyrazole-3-carboxamido)benzamido)phenyl)acetic acid |
| 157 | 2-(5-fluoro-2-(4-(piperidin-1-yl)-3-(2-(1-(2,2,2-trifluoroethyl)-1H-indazol-3- |
| | yl)acetamido)benzamido)phenyl)acetic acid |
| 158 | 2-(5-fluoro-2-(4-(piperidin-1-yl)-3-(1-(2,2,2-trifluoroethyl)-1H-indazole-3- |
| | carboxamido)benzamido)phenyl)acetic acid; |
| 159 | 2-(2-(3-(1-benzyl-1H-indazole-3-carboxamido)-4-(piperidin-1-yl)benzamido)-5- |
| | fluorophenyl)acetic acid; |
| 160 | 2-(2-(3-(1-ethyl-1H-indazole-3-carboxamido)-4-(piperidin-1-yl)benzamido)-5- |
| | fluorophenyl)acetic acid; |
| 161 | 2-(2-(4-(piperidin-1-yl)-3-(4,5,6,7-tetrahydropyrazolo[1,5-a]pyridine-2- |
| | carboxamido)benzamido)phenyl)acetic acid; |
| 162 | 2-(5-fluoro-2-(3-(1-isopropyl-1H-indazole-3-carboxamido)-4-(piperidin-1- |
| | yl)benzamido)phenyl)acetic acid; |
| 163 | 2-(2-(3-(1-methyl-5-phenyl-1H-pyrazole-3-carboxamido)-4-(piperidin-1- |
| | yl)benzamido)phenyl)acetic acid; |
| 164 | N-(5-((4-(1H-tetrazol-5-yl)phenyl)carbamoyl)-2-(piperidin-1-yl)phenyl)-1-(2,2,2- |
| | trifluoroethyl)-1H-indazole-3-carboxamide; |
| 165 | N-(5-((4-(1H-tetrazol-5-yl)phenyl)carbamoyl)-2-(piperidin-1-yl)phenyl)-6-fluoro-1- |
| | methyl-1H-indazole-3-carboxamide; |
| 166 | N-(5-((2-fluoro-5-(1H-tetrazol-5-yl)phenyl)carbamoyl)-2-(piperidin-1-yl)phenyl)-1- |
| | methyl-1H-indazole-3-carboxamide; |
| 167 | 5-(6-(1H-tetrazol-5-yl)-2H-indazol-2-yl)-2-(piperidin-1-yl)-N-(p-tolyl)benzamide; |
| 168 | N-(4-methylphenyl)-5-({[4-(5-oxo-4,5-dihydro-1,2,4-thiadiazol-3- |
| | yl)phenyl]methyl}amino)-2-(piperidin-1-yl)benzamide; |
| 169 | 5-((2-fluoro-5-(1H-tetrazol-5-yl)benzyl)amino)-2-(piperidin-1-yl)-N-(p- |
| | tolyl)benzamide; |
| 170 | 5-(1-oxo-5-(1H-tetrazol-5-yl)isoindolin-2-yl)-2-(piperidin-1-yl)-N-(p-tolyl)benzamide; |
| 172 | 1-ethyl-N-(5-((2-(2-(methylsulfonamido)-2-oxoethyl)phenyl)carbamoyl)-2-(piperidin-1- |
| | yl)phenyl)-1H-indazole-3-carboxamide; |
| 173 | 2-(2-(3-(1-methyl-1H-indole-2-carboxamido)-4-(piperidin-1- |
| | yl)benzamido)phenyl)acetic acid; |
| 174 | 2-(2-(3-(1-methyl-1H-indazole-3-carboxamido)-4-(piperidin-1- |
| | yl)benzamido)phenyl)acetic acid; |
| 175 | 5-((4-(1H-tetrazol-5-yl)benzyl)amino)-2-(piperidin-1-yl)-N-(p-tolyl)benzamide; and |
| 176 | 2-((3-((3-chloro-4-methylphenyl)carbamoyl)-4- |
| | (cyclopentylamino)phenyl)carbamoyl)benzoic acid |

or a pharmaceutically acceptable salt, isomer, solvate, prodrug, or tautomer thereof

In some embodiments, the compound of Formula I is selected from:

| Compound | |
|---|---|
| No. | Compound Name |
| 7 | 2-fluoro-2-(5-fluoro-2-(4-(piperidin-1-yl)-3-(1-(2,2,2-trifluoroethyl)-1H- |
| | indazole-3-carboxamido)benzamido)phenyl)acetic acid |
| 30 | 2-(5-fluoro-2-(4-(piperidin-1-yl)-3-(1-(2,2,2-trifluoroethyl)-1H-indazole-3- |
| | carboxamido)benzamido)phenyl)-2-methylpropanoic acid |
| 31 | 1-(5-fluoro-2-(4-(piperidin-1-yl)-3-(1-(2,2,2-trifluoroethyl)-1H-indazole-3- |
| | carboxamido)benzamido)phenyl)cyclopropane-1-carboxylic acid |
| 73 | 2-(5-fluoro-2-(4-(3-methylpiperidin-1-yl)-3-(1-(2,2,2-trifluoroethyl)-1H- |
| | indazole-3-carboxamido)benzamido)phenyl)acetic acid |
| 122 | 2-(5-fluoro-2-(4-(piperidin-1-yl)-3-(1-(prop-2-yn-1-yl)-1H-indazole-3- |
| | carboxamido)benzamido)phenyl)acetic acid |
| 22 | 2-(2-(4-(piperidin-1-yl)-3-(1-(2,2,2-trifluoroethyl)-1H-indazole-3- |
| | carboxamido)benzamido)-5-(trifluoromethyl)phenyl)acetic acid |
| 28 | 2-(5-fluoro-2-(4-(piperidin-1-yl)-3-(1-(2,2,2-trifluoroethyl)-1H-indazole-3- |
| | carboxamido)benzamido)phenyl)butanoic acid |
| 61 | 2-(2-(4-(4,4-dimethylpiperidin-1-yl)-3-(1-(2,2,2-trifluoroethyl)-1H-indazole- |
| | 3-carboxamido)benzamido)-5-fluorophenyl)acetic acid |
| 104 | 2-(5-fluoro-2-(4-(piperidin-1-yl)-3-(1-((tetrahydro-2H-pyran-4-yl)methyl)-1H- |
| | indazole-3-carboxamido)benzamido)phenyl)acetic acid |
| 121 | 2-(5-fluoro-2-(4-(piperidin-1-yl)-3-(1-(3,3,3-trifluoropropyl)-1H-indazole-3- |
| | carboxamido)benzamido)phenyl)acetic acid |
| 40 | 2-(2-(4-(4,4-difluoropiperidin-1-yl)-3-(1-(2,2,2-trifluoroethyl)-1H-indazole-3- |
| | carboxamido)benzamido)-5-fluorophenyl)acetic acid |
| 25 | 2-(3-chloro-2-(4-(piperidin-1-yl)-3-(1-(2,2,2-trifluoroethyl)-1H-indazole-3- |
| | carboxamido)benzamido)phenyl)acetic acid |
| 123 | 2-(2-(3-(1-(but-2-yn-1-yl)-1H-indazole-3-carboxamido)-4-(piperidin-1- |
| | yl)benzamido)-5-fluorophenyl)acetic acid |
| 158 | 2-(5-fluoro-2-(4-(piperidin-1-yl)-3-(1-(2,2,2-trifluoroethyl)-1H-indazole-3- |
| | carboxamido)benzamido)phenyl)acetic acid; |
| 159 | 2-(2-(3-(1-benzyl-1H-indazole-3-carboxamido)-4-(piperidin-1-yl)benzamido)-5- |
| | fluorophenyl)acetic acid; |
| 160 | 2-(2-(3-(1-ethyl-1H-indazole-3-carboxamido)-4-(piperidin-1-yl)benzamido)-5- |
| | fluorophenyl)acetic acid; |
| 161 | 2-(2-(4-(piperidin-1-yl)-3-(4,5,6,7-tetrahydropyrazolo[1,5-a]pyridine-2- |
| | carboxamido)benzamido)phenyl)acetic acid; |
| 162 | 2-(5-fluoro-2-(3-(1-isopropyl-1H-indazole-3-carboxamido)-4-(piperidin-1- |
| | yl)benzamido)phenyl)acetic acid; and |
| 163 | 2-(2-(3-(1-methyl-5-phenyl-1H-pyrazole-3-carboxamido)-4-(piperidin-1- |
| | yl)benzamido)phenyl)acetic acid |

or a pharmaceutically acceptable salt, isomer, solvate, prodrug, or tautomer thereof.

It should be understood that all isomeric forms are included within the present disclosure, including mixtures thereof. If the compound contains a double bond, the substituent may be in the E or Z configuration. If the compound contains a disubstituted cycloalkyl, the cycloalkyl substituent may have a cis- or trans configuration. All tautomeric forms are also intended to be included.

Compounds of the present disclosure, and pharmaceutically acceptable salts, hydrates, solvates, stereoisomers and prodrugs thereof may exist in their tautomeric form (for example, as an amide or imino ether). All such tautomeric forms are contemplated herein as part of the present disclosure.

The compounds of the present disclosure may contain asymmetric or chiral centers, and, therefore, exist in different stereoisomeric forms. It is intended that all stereoisomeric forms of the compounds of the present disclosure as well as mixtures thereof, including racemic mixtures, form part of the present disclosure. In addition, the present disclosure embraces all geometric and positional isomers. For example, if a compound of the present disclosure incorporates a double bond or a fused ring, both the cis- and trans-forms, as well as mixtures, are embraced within the scope of the present disclosure. each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the present disclosure may be atropisomers (e.g., substituted biaryls) and are considered as part of this present disclosure. Enantiomers can also be separated by use of a chiral HPLC column.

It is also possible that the compounds of the present disclosure may exist in different tautomeric forms, and all such forms are embraced within the scope of the present disclosure. Also, for example, all keto-enol and imine-enamine forms of the compounds are included in the present disclosure.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this present disclosure, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the present disclosure. Also, for example, all keto-enol and imine-enamine forms of the compounds are included in the present disclosure). Individual stereoisomers of the compounds of the present disclosure may, for example, be substantially free of other isomers, or may be admixed, for example, as racemates or with all other, or other selected, stereoisomers. The chiral centers of the present disclosure can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The compounds of Formula I may form salts which are also within the scope of this present disclosure. Reference to a compound of the Formula herein is understood to include reference to salts thereof, unless otherwise indicated.

The present disclosure relates to compounds which are LPL activators. In one embodiment, the compounds of the present disclosure are LPL activators.

The present disclosure is directed to compounds as described herein and pharmaceutically acceptable salts, hydrates, solvates, prodrugs, stereoisomers, or tautomers thereof, and pharmaceutical compositions comprising one or more compounds as described herein, or pharmaceutically acceptable salts, hydrates, solvates, prodrugs, stereoisomers, or tautomers thereof

### Method of Synthesizing the Compounds

The compounds of the present disclosure may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (I). Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below. Compounds of the present disclosure can be synthesized by following the steps outlined in General Procedures I-XII which comprise different sequences of assembling intermediates or compounds. Starting materials are either commercially available or made by known procedures in the reported literature or as illustrated below.

### General Procedure I

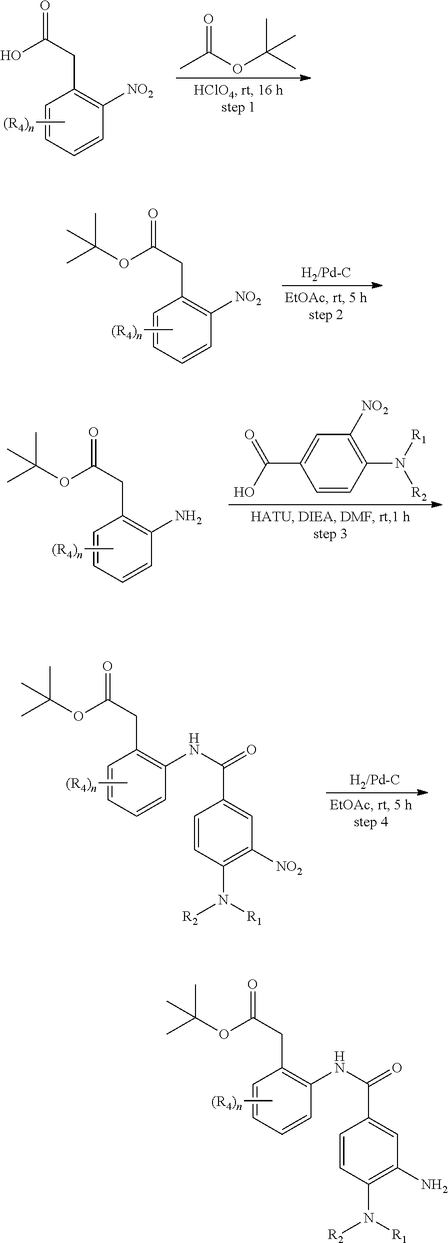

### General Procedure II

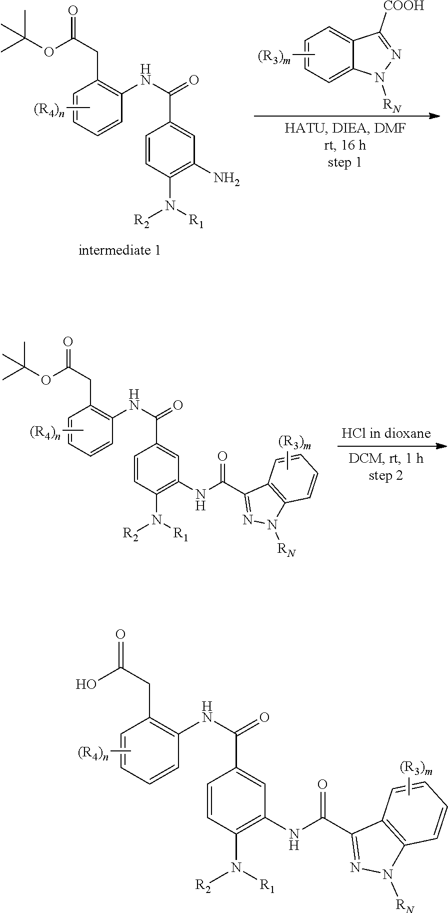

### General Procedure III

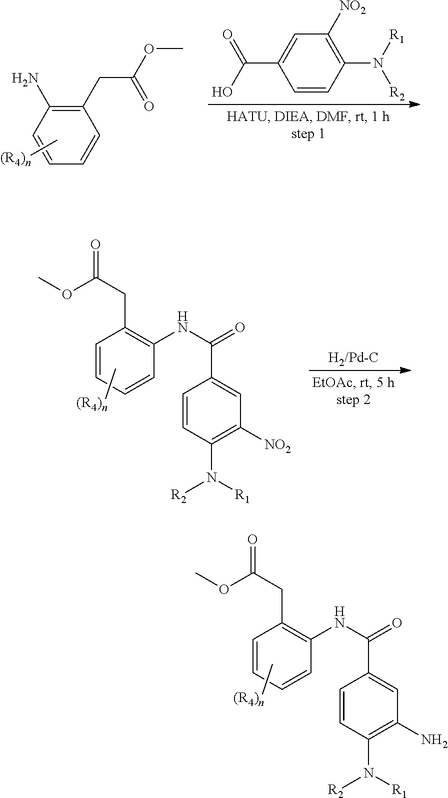

### General Procedure IV

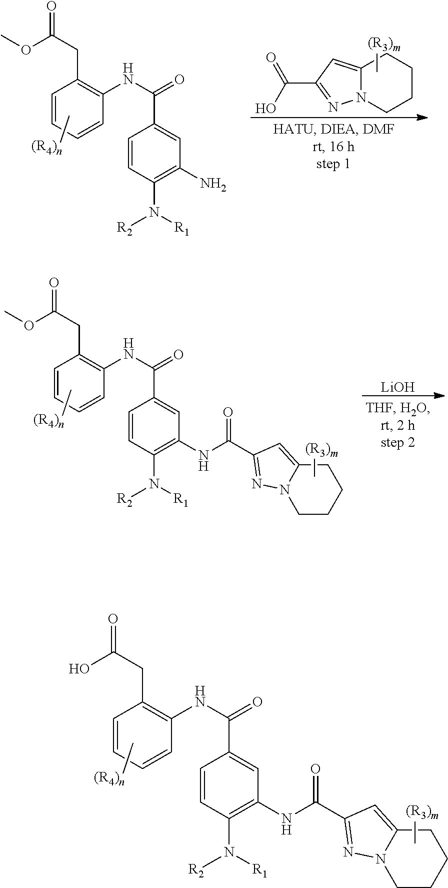

### General Procedure V

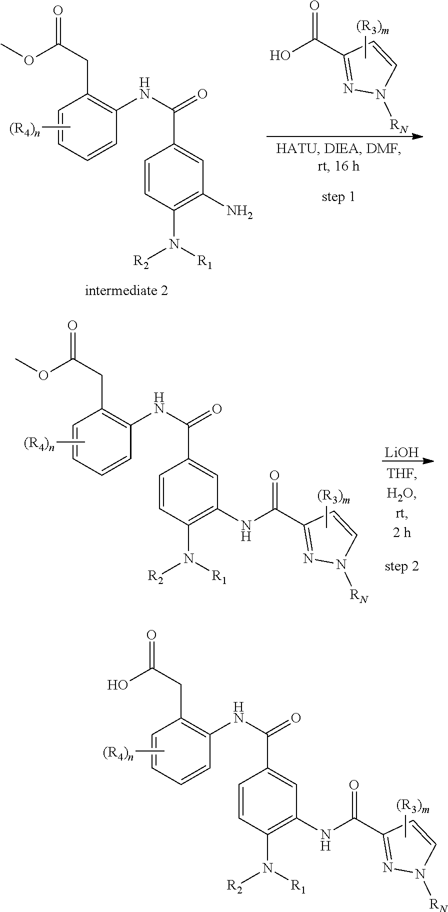

### General Procedure VI

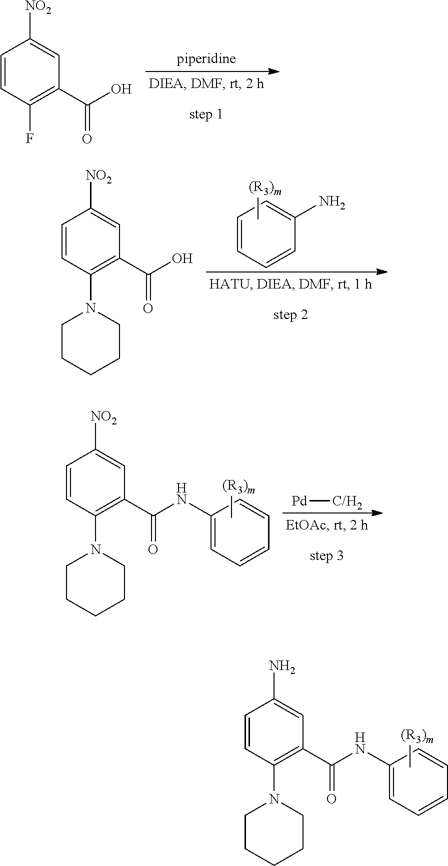

### General Procedure VII

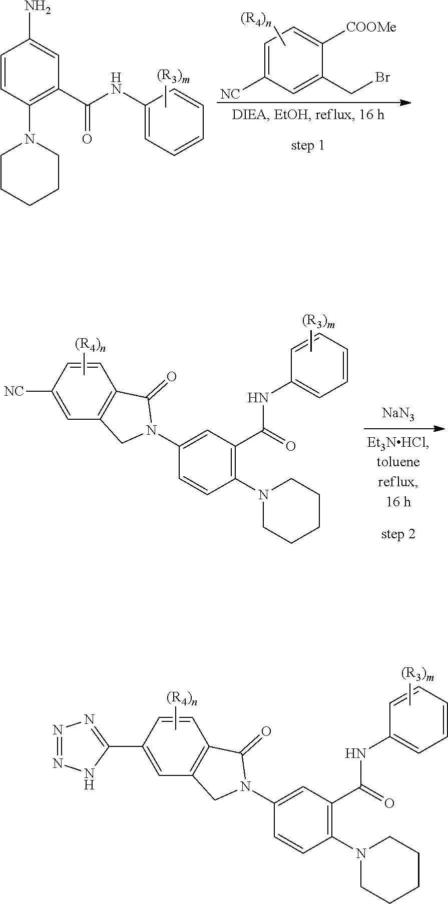

### General Procedure VIII

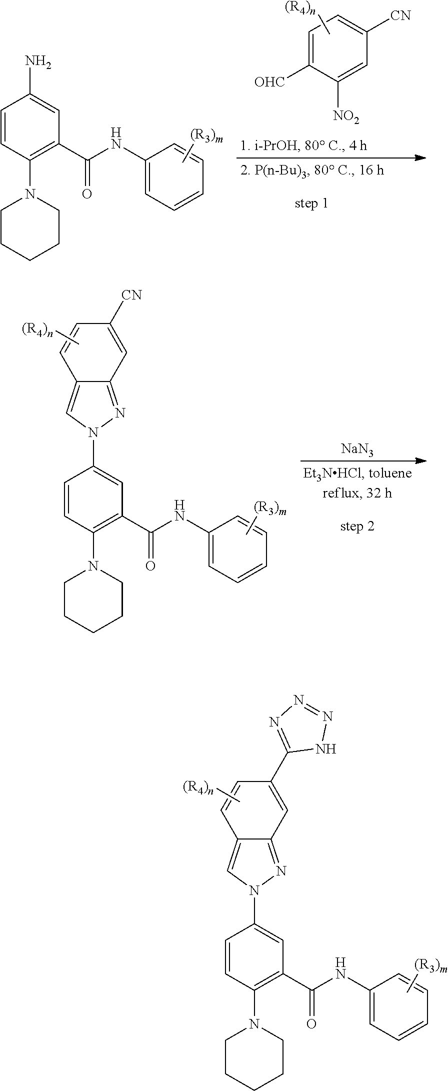

### General Procedure IX

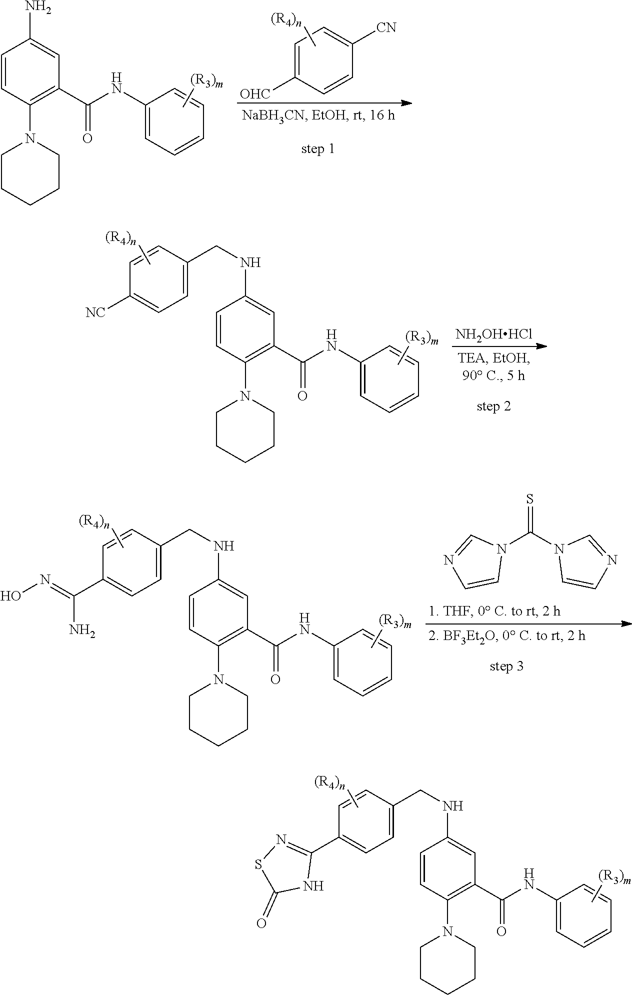

### General Procedure X

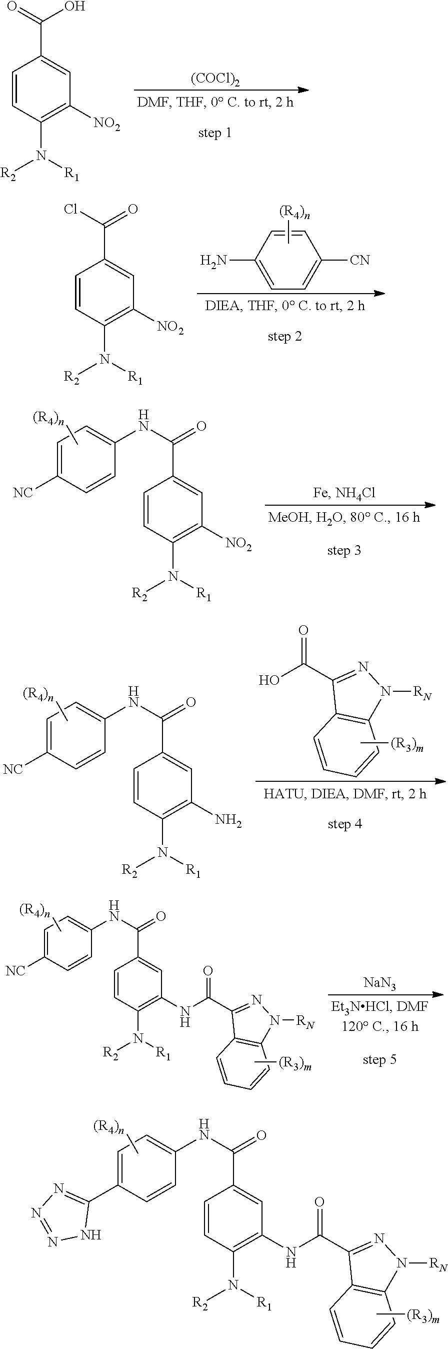

### General Procedure XI

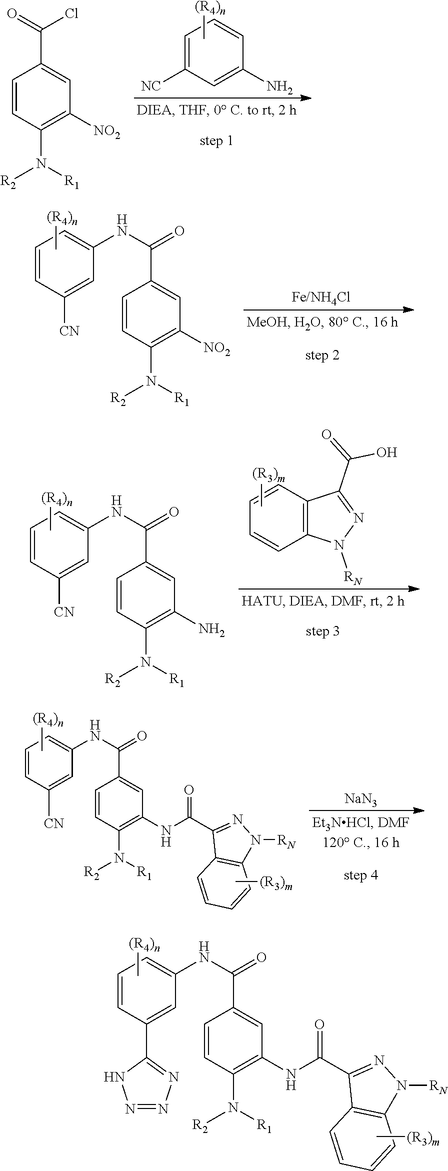

### General Procedure XII

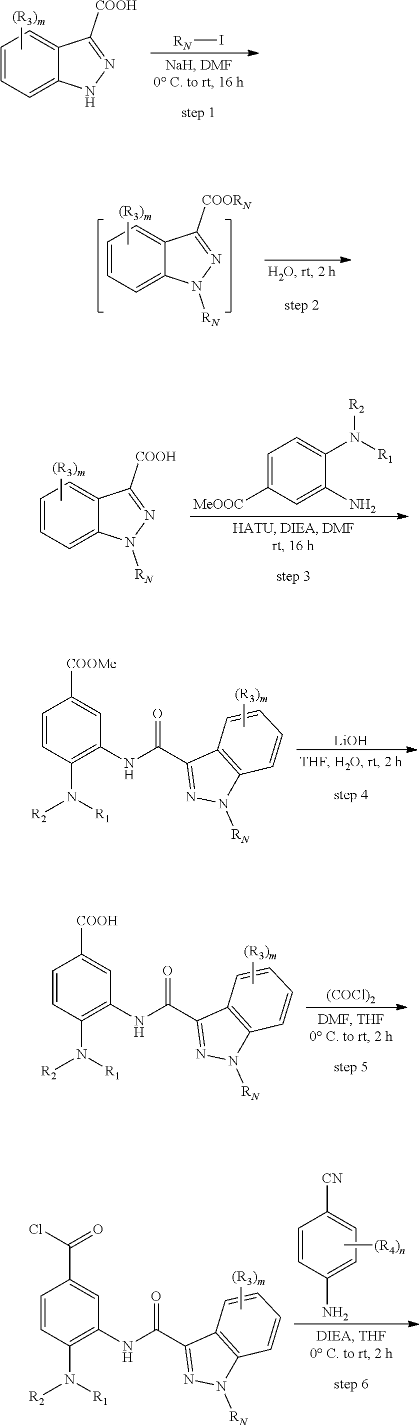

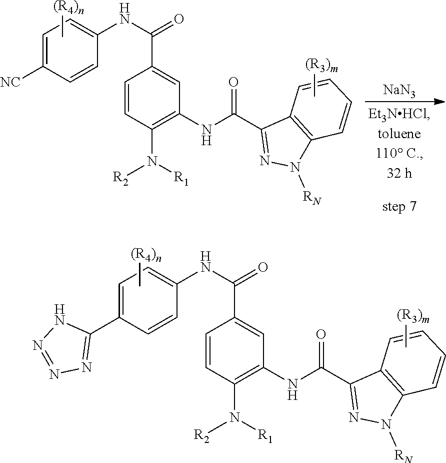

### Methods of Using the Disclosed Compounds

Another aspect of the present disclosure relates to a method of treating a condition associated with activation of LPL. The method comprises administering to a patient in need of a treatment for conditions associated with activation of LPL an effective amount the compositions and compounds of Formula (I).

In another aspect, the present disclosure is directed to a method of activating LPL. The method involves administering to a patient in need thereof an effective amount of a compound of Formula (I).

Another aspect of the present disclosure relates to a method of treating, preventing, inhibiting or eliminating a condition in a patient associated with the activation of LPL, the method comprising administering to a patient in need thereof an effective amount of a compound of Formula (I). In one embodiment, the disease or condition may be, but not limited to, hypertriglyceridemia, moderate hypertriglyceridemia, severe hypertriglyceridemia, mixed dyslipidemia, familial chylomicronemia, type 1 hyperlipoproteinemia, type IIb hyperlipoproteinemia, type III hyperlipoproteinemia, type IV hyperlipoproteinemia, type V hyperlipoproteinemia, metabolic syndrome, partial lipodystrophy, prediabetes, type II diabetes, atherosclerotic cardiovascular disease, myocardial infarction, coronary heart disease, ischemic heart disease, peripheral vascular disease, cerebrovascular disease, peripheral arterial disease, acute pancreatitis.

The present disclosure also relates to the use of an activator of LPL the preparation of a medicament used in the treatment, prevention, inhibition or elimination of a disease or condition mediated by LPL, wherein the medicament comprises a compound of Formula (I).

In another aspect, the present disclosure relates to a method for the manufacture of a medicament for treating, preventing, inhibiting, or eliminating a disease or condition mediated by LPL, wherein the medicament comprises a compound of Formula (I).

Another aspect of the present disclosure relates to a compound of Formula (I) for use in the manufacture of a medicament for treating a disease associated with activating LPL.

In another aspect, the present disclosure relates to the use of a compound of Formula (I) in the treatment of a disease associated with activating LPL.

Another aspect of the present disclosure relates to a method of treating hypertriglyceridemia. The method comprises administering to a patient in need thereof an effective amount of a compound of Formula (I).

Another aspect of the present disclosure relates to a method of preventing hypertriglyceridemia. The method comprises administering to a patient in need thereof an effective amount of a compound of Formula (I).

Another aspect of the present disclosure relates to a method of treating moderate hypertriglyceridemia. The method comprises administering to a patient in need thereof an effective amount of a compound of Formula (I).

Another aspect of the present disclosure relates to a method of preventing moderate hypertriglyceridemia. The method comprises administering to a patient in need thereof an effective amount of a compound of Formula (I).

Another aspect of the present disclosure relates to a method of treating severe hypertriglyceridemia. The method comprises administering to a patient in need thereof an effective amount of a compound of Formula (I).

Another aspect of the present disclosure relates to a method of preventing severe hypertriglyceridemia. The method comprises administering to a patient in need thereof an effective amount of a compound of Formula (I).

Another aspect of the present disclosure relates to a method of treating mixed dyslipidemia.

The method comprises administering to a patient in need thereof an effective amount of a compound of Formula (I).

Another aspect of the present disclosure relates to a method of preventing mixed dyslipidemia. The method comprises administering to a patient in need thereof an effective amount of a compound of Formula (I).

Another aspect of the present disclosure relates to a method of treating familial chylomicronemia. The method comprises administering to a patient in need thereof an effective amount of a compound of Formula (I).

Another aspect of the present disclosure relates to a method of preventing familial chylomicronemia. The method comprises administering to a patient in need thereof an effective amount of a compound of Formula (I).

Another aspect of the present disclosure relates to a method of treating type 1 hyperlipoproteinemia. The method comprises administering to a patient in need thereof an effective amount of a compound of Formula (I).

Another aspect of the present disclosure relates to a method of preventing type 1 hyperlipoproteinemia. The method comprises administering to a patient in need thereof an effective amount of a compound of Formula (I).

Another aspect of the present disclosure relates to a method of treating type IIb hyperlipoproteinemia. The method comprises administering to a patient in need thereof an effective amount of a compound of Formula (I).

Another aspect of the present disclosure relates to a method of preventing type IIb hyperlipoproteinemia. The method comprises administering to a patient in need thereof an effective amount of a compound of Formula (I).

Another aspect of the present disclosure relates to a method of treating type III hyperlipoproteinemia. The method comprises administering to a patient in need thereof an effective amount of a compound of Formula (I).

Another aspect of the present disclosure relates to a method of preventing type III hyperlipoproteinemia. The method comprises administering to a patient in need thereof an effective amount of a compound of Formula (I).

Another aspect of the present disclosure relates to a method of treating type IV hyperlipoproteinemia. The method comprises administering to a patient in need thereof an effective amount of a compound of Formula (I).

Another aspect of the present disclosure relates to a method of preventing type IV hyperlipoproteinemia. The method comprises administering to a patient in need thereof an effective amount of a compound of Formula (I).

Another aspect of the present disclosure relates to a method of treating type V hyperlipoproteinemia. The method comprises administering to a patient in need thereof an effective amount of a compound of Formula (I).

Another aspect of the present disclosure relates to a method of preventing type V hyperlipoproteinemia. The method comprises administering to a patient in need thereof an effective amount of a compound of Formula (I).

Another aspect of the present disclosure relates to a method of treating metabolic syndrome. The method comprises administering to a patient in need thereof an effective amount of a compound of Formula (I).

Another aspect of the present disclosure relates to a method of preventing metabolic syndrome. The method comprises administering to a patient in need thereof an effective amount of a compound of Formula (I).

Another aspect of the present disclosure relates to a method of treating partial lipodystrophy. The method comprises administering to a patient in need thereof an effective amount of a compound of Formula (I).

Another aspect of the present disclosure relates to a method of preventing partial lipodystrophy. The method comprises administering to a patient in need thereof an effective amount of a compound of Formula (I).

Another aspect of the present disclosure relates to a method of treating type IIb prediabetes.

The method comprises administering to a patient in need thereof an effective amount of a compound of Formula (I).

Another aspect of the present disclosure relates to a method of preventing type IIb prediabetes. The method comprises administering to a patient in need thereof an effective amount of a compound of Formula (I).

Another aspect of the present disclosure relates to a method of treating type II diabetes. The method comprises administering to a patient in need thereof an effective amount of a compound of Formula (I).

Another aspect of the present disclosure relates to a method of type II diabetes. The method comprises administering to a patient in need thereof an effective amount of a compound of Formula (T).

Another aspect of the present disclosure relates to a method of treating atherosclerotic cardiovascular disease. The method comprises administering to a patient in need thereof an effective amount of a compound of Formula (I).

Another aspect of the present disclosure relates to a method of preventing atherosclerotic cardiovascular disease. The method comprises administering to a patient in need thereof an effective amount of a compound of Formula (I).

Another aspect of the present disclosure relates to a method of treating myocardial infarction. The method comprises administering to a patient in need thereof an effective amount of a compound of Formula (I).

Another aspect of the present disclosure relates to a method of preventing myocardial infarction. The method comprises administering to a patient in need thereof an effective amount of a compound of Formula (I).

Another aspect of the present disclosure relates to a method of treating coronary heart disease. The method comprises administering to a patient in need thereof an effective amount of a compound of Formula (I).

Another aspect of the present disclosure relates to a method of preventing coronary heart disease. The method comprises administering to a patient in need thereof an effective amount of a compound of Formula (I).

Another aspect of the present disclosure relates to a method of treating ischemic heart disease. The method comprises administering to a patient in need thereof an effective amount of a compound of Formula (I).

Another aspect of the present disclosure relates to a method of preventing ischemic heart disease. The method comprises administering to a patient in need thereof an effective amount of a compound of Formula (I).

Another aspect of the present disclosure relates to a method of treating peripheral vascular disease. The method comprises administering to a patient in need thereof an effective amount of a compound of Formula (I).

Another aspect of the present disclosure relates to a method of preventing peripheral vascular disease. The method comprises administering to a patient in need thereof an effective amount of a compound of Formula (I).

Another aspect of the present disclosure relates to a method of treating cerebrovascular disease. The method comprises administering to a patient in need thereof an effective amount of a compound of Formula (I).

Another aspect of the present disclosure relates to a method of preventing cerebrovascular disease. The method comprises administering to a patient in need thereof an effective amount of a compound of Formula (I).

Another aspect of the present disclosure relates to a method of treating peripheral arterial disease. The method comprises administering to a patient in need thereof an effective amount of a compound of Formula (I).

Another aspect of the present disclosure relates to a method of preventing peripheral arterial disease. The method comprises administering to a patient in need thereof an effective amount of a compound of Formula (I).

Another aspect of the present disclosure relates to a method of treating acute pancreatitis. The method comprises administering to a patient in need thereof an effective amount of a compound of Formula (I).

Another aspect of the present disclosure relates to a method of preventing acute pancreatitis. The method comprises administering to a patient in need thereof an effective amount of a compound of Formula (I).

In one embodiment, the present disclosure relates to the use of an activator of LPL for the preparation of a medicament used in treatment, prevention, inhibition or elimination of a condition selected from hypertriglyceridemia, moderate hypertriglyceridemia, severe hypertriglyceridemia, mixed dyslipidemia, familial chylomicronemia, type 1 hyperlipoproteinemia, type IIb hyperlipoproteinemia, type III hyperlipoproteinemia, type IV hyperlipoproteinemia, type V hyperlipoproteinemia, metabolic syndrome, partial lipodystrophy, prediabetes, type II diabetes, atherosclerotic cardiovascular disease, myocardial infarction, coronary heart disease, ischemic heart disease, peripheral vascular disease, cerebrovascular disease, peripheral arterial disease, and acute pancreatitis.

In one embodiment, the subject is a mammal.

In one embodiment, the mammal is a human.

In one embodiment, the subject has high polygenic risk for coronary heart disease.

In one embodiment, the subject has high polygenic risk for hypertriglyceridemia.

In one embodiment, the subject has high polygenic risk for lipodystrophy.

In one embodiment, the subject has high polygenic risk for peripheral vascular disease.

In one embodiment, the subject has high polygenic risk for cerebrovascular disease.

In one embodiment, the subject has rare damaging heterozygous or homozygous mutations in one or more, two or more, three or more, four or more, five or more, or six or more of the genes selected from the group consisting of LPL, APOC2, APOA5, IMFI, GPIHBPI, APOF., and APOB.

The disclosed compounds of the present disclosure can be administered in effective amounts to treat or prevent a disorder and/or prevent the development thereof in subjects.

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices.

Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a compound of the present disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564 which is hereby incorporated by reference in its entirety.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the present disclosure is directed to pharmaceutical compositions comprising a compound of Formula (I) and a pharmaceutically acceptable carrier. The pharmaceutical acceptable carrier may further include an excipient, diluent, or surfactant. In some embodiments, the pharmaceutical composition can further comprise an additional pharmaceutically active agent.

In one embodiment, the pharmaceutical acceptable carrier further comprises an excipient, diluent, surfactant, or any combination thereof.

In one embodiment, the pharmaceutical composition further comprises at least one additional therapeutic agent.

Another aspect of the present disclosure is directed to pharmaceutical compositions for the treatment or prevention of a condition comprising a compound of Formula (I), or a pharmaceutically acceptable salt, ester, or amino acid conjugate thereof.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 1, 5, 20, 50, 75, 100, 150, 250, 500, 750, 1000, 1250, 2000, 2500, 3500, or 5000 mg of the disclosed compound, or, in a range of from one amount to another amount in the list of doses. In one embodiment, the compositions are in the form of a tablet that can be scored.

In one embodiment, the composition comprises about 1 mg to about 2000 mg of the compound.

In one embodiment, the composition is administered to the subject twice daily, once daily, once every other day, or once weekly.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

The compounds of the present disclosure may be prepared by use of known chemical reactions and procedures. Nevertheless, the following general preparative methods are presented to aid the reader in synthesizing the LPL activators with specific details provided below in the experimental section to illustrate working examples.

All variable groups of these methods are as described in the generic description if they are not specifically defined below.

It is recognized that compounds of the disclosure with each claimed optional functional group may not be prepared by each of the below-listed methods. Within the scope of each method, optional substituents may appear on reagents or intermediates which may act as protecting or otherwise non-participating groups. Utilizing methods well known to those skilled in the art, these groups are introduced and/or removed during the course of the synthetic schemes which provide the compounds of the present disclosure.

Abbreviations used in the following examples and elsewhere herein are:
(Bu₃Sn)₂O Bis(tributyltin) oxide
Cs₂CO₃ Caesium carbonate
DABCO 1,4-diazabicyclo[2.2.2]octane
DMAc Dimethylacetamide
DCM Dichloromethane
DIPEA N,N-Diisopropylethylamine
DMAc N,N-Dimethylacetamide
DME Dimethoxyethane
DMF N,N-Dimethylformamide
DMSO Dimethylsulfoxide
DPPP Propane-1,3-diylbis(diphenylphosphane)
Eq Equivalent
EtOAc Ethyl acetate
EtOH Ethanol
HATU (1-[Bis(dimethylamino)methylene]-1H-1,2,3-triazolo[4,5-b]pyridinium-3-oxide hexafluorophosphate
HCl Hydrochloric acid
Ir[dF(CF₃)ppy]₂(dtbpy)(PF₆) (4,4′-Di-t-butyl-2,2′-bipyridine)bis[3,5-difluoro-2-[5-trifluoromethyl-2-pyridinyl-kN)phenyl-kC]iridium(11) hexafluorophosphate
HPLC High Performance Liquid Chromatography
K₃PO₄ Potassium phosphate
LC Liquid chromatography
LiOH Lithium hydroxide
M Molar
MeCN Acetonitrile
MeOH Methanol
MS Mass spectrometry
MsCl Methanesulfonyl chloride
N Normal
Na₂CO₃ Sodium carbonate
NaH Sodium hydride
NaHCO₃ Sodium bicarbonate
Na₂SO₄ Sodium sulfate
NH₄Cl Ammonium chloride
NiCl₂.dtbbpy Bis(1,1-dimethylethyl)-2,2′-bipyridine]nickel (U) dichloride
NMR Nuclear magnetic resonance
Pd/C Palladium on carbon
Pd₂(dba)₃ Tris(dibenzylideneacetone)dipalladium(O)
Pd(dppf)Cl₂ 1,1′-Bis(diphenylphosphino)ferrocene]dichloropalladium(II)
Pd(OAc)₂ Palladium(II) acetate
PtO₂ Platinum oxide
Py Pyridine
rt Room temperature
RP Reverse-phase
TEA Triethylamine
TFA Trifluoroacetic acid
THF Tetrahydrofuran
T₃P Propanephosphonic acid anhydride
TTMSS Tris(trimethylsilyl)silane
UPLC Ultra Performance Liquid Chromatography
Xantphos 9,9-Dimethyl-9H-xanthene-4,5-diyl)bis(diphenylphosphane

Instruments and analytical methods used herein are:

| | LCMS | | |
|---|---|---|---|
| | No. | Column | Method |
| LCMS, | LCMS-B | Kinetex EVO | Method: 5-95AB: Instrument: Agilent |
| acidic | LCMS-D | C18, 2.1*30 mm, | 1200\G6110A; eluent A: water + 0.0375 vol % |
| condition | LCMS-G | 5 um | trifluoroacetic acid, eluent B: acetonitrile + |
| | | | 0.01875 vol % trifluoroacetic acid; gradient: 0- |
| | | | 0.8 min, 5-95% B, 0.8-1.2 min 95% B; flow 1.5 |
| | | | ml/min; temperature: 50° C.; PDA: 220 nm & |
| | | | 254 nm. |
| | LCMS-L | Kinetex EVO | Method: 5-95AB: Instrument: SHIMADZU |
| | LCMS-N | C18, 2.1*30 mm, | LCMS-2020; eluent A: water + 0.0375 vol % |
| | LCMS-X | 5 um | trifluoroacetic acid, eluent B: acetonitrile + |
| | LCMS-Q | | 0.01875 vol % trifluoroacetic acid; gradient: 0- |
| | LCMS-AJ | | 0.8 min, 5-95% B, 0.8-1.2 min 95% B; flow 1.5 |
| | LCMS-AG | | ml/min; temperature: 50° C.; PDA: 220 nm & |
| | LCMS-P | | 254 nm. |
| | LCMS-S | | Method: 0-60AB: Instrument: SHIMADZU |
| | LCMS-A | | LCMS-2020; eluent A: water + 0.0375 vol % |
| | LCMS-I | | trifluoroacetic acid, eluent B: acetonitrile + |
| | LCMS-R | | 0.01875 vol % trifluoroacetic acid; gradient: 0- |
| | LCMS-H | | 0.8 min, 0-60% B, 0.8-1.2 min 60% B; flow 1.5 |
| | LCMS-Y | | ml/min; temperature: 50° C.; PDA: 220 nm & |
| | LCMS-AA | | 254 nm. |
| | LCMS-AB | | |
| | LCMS-AH | | |
| | LCMS-J | | |
| | LCMS-T | | |
| | LCMS-AI | | |
| | LCMS-AN | | |
| | LCMS-AO | | |
| LCMS, | LCMS-M | Kinetex EVO | Method: 5-95CD: Instrument: SHIMADZU |
| basic | LCMS-W | C18, 2.1*30 mm, | LCMS-2020; eluent A: 0.025% NH3•H2O in |
| condition | LCMS-O | 5 um | water(v/v), eluent B: acetonitrile; gradient: 0-0.8 |
| | LCMS-K | | min, 5-95% B, 0.8-1.2 min 95% B; flow 1.5 |
| | LCMS-C | XBridge C18, | ml/min; temperature: 50° C.; PDA: 220 nm & |
| | | 2.1*50 mm, 5 um | 254 nm. |
| HPLC, | HPLC-L | Kinetex EVO | Method: 10-80AB: Instrument: SHIMADZU |
| acidic | HPLC-BK | C18, 3.0*50 mm, | LC-20AB; eluent A: water + 0.0375 vol % |
| condition | HPLC-S | 2.6 um | trifluoroacetic acid, eluent B: acetonitrile + |
| | HPLC-P | Kinetex EVO | 0.01875 vol % trifluoroacetic acid; gradient: 0- |
| | HPLC-BH | C18, 4.6*50 mm, | 2.4 min, 10-80% B, 2.4-3.7 min 80% B; flow 1.5 |
| | HPLC-BL | 5 um | ml/min; temperature: 50° C.; PDA: 220 nm & |
| | HPLC-BM | | 215 nm & 254 nm. |
| | HPLC-Q | | |
| | HPLC-M | | |
| | HPLC-N | | |
| | HPLC-R | | |
| | HPLC-AH | | |
| | HPLC-E | | |
| | HPLC-D | | |
| | HPLC-H | | |
| HPLC, | HPLC-O | XBridge C18, | Method: 10-80CD: Instrument: SHIMADZU |
| basic | | 3.0*50 mm, 3.5 um | LC-20AB; eluent A: 0.025% NH3•H2O in |
| condition | HPLC-C | XBridge C18, | water(v/v), eluent B: acetonitrile; gradient: 0- |
| | HPLC-B | 2.1*50 mm 5 um | 3.0 min, 10-80% B, 3.0-3.5 min 80% B; flow |
| | HPLC-J | | 1.2 ml/min; temperature: 50° C.; PDA: 220 nm |
| | HPLC-AG | | & 215 nm & 254 nm. |

### Preparative HPLC

### Method 1:

Instrument: pump: Labomatic HD-5000 or HD-3000, head HDK 280, low-pressure gradient module ND-B1000; manual injection valve: Rheodyne 3725i038; detector: Knauer Azura UVD 2.15; collector: Labomatic Labocol Vario-4000; column: Chromatorex RP C-18 10 μm, 125×30 mm; eluent acidic/basic; gradient B—F; UV-detection: 210-254 nm.

Eluent acidic: solvent A: water+0.1 vol-% formic acid, solvent B: acetonitrile

Eluent basic: solvent A: water+0.2 vol-% ammonia (32%), solvent B: acetonitrile

Gradient B: 0.00-0.50 min 40% B (150 ml/min), 0.50-6.00 min 40-70% B (150 ml/min), 6.00-6.05 min 70-100% B (150 ml/min), 6.05-8.00 min 100% B (150 ml/min)

Gradient C: 0.00-2.50 min 15% B (150 ml/min), 2.50-8.00 min 15-55% B (150 ml/min), 8.00-8.05 min 55-100% B (150 ml/min), 8.05-10.05 min 100% B (150 ml/min)

Gradient D: 0.00-5.00 min 30% B (60 ml/min), 5.00-17.00 min 30-70% B (60 ml/min), 17.00-17.10 min 70-100% B (60 ml/min), 17.10-20.00 min 100% B (60 ml/min)

Gradient E: 0.00-2.50 min 40% B (150 ml/min), 2.50-8.00 min 40-80% B (150 ml/min), 8.00-8.10 min 80-100% B (150 ml/min), 8.10-10.00 min 100% B (150 ml/min)

Gradient F: 0.00-2.50 min 65% B (150 ml/min), 2.50-8.00 min 65-100% B (150 ml/min), 8.00-10.00 min 100% B (150 ml/min)

### Analytical LCMS

### UPLC-MS

### Method 1:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 999% B; flow 0.8 ml/min; temperature: 60° C.; DAD scan: 210-400 nm.

### Method 2:

Instrument: Waters Acquity UPLCMS SingleQuad; Column: Acquity UPLC BEH C18 1.7 μm, 50×2. 1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

### Method 3:

Instrument: Waters Acquity UPLCMS SingleQuad; Column: Acquity UPLC BEH C18 1.7 μm, 50×2. 1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

### Method 4:

Instrument: Waters Acquity UPLCMS SingleQuad; Column: Acquity UPLC BEH C18 1.7 μm, 50×2. 1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

### Method 5:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 m/min; temperature: 60° C.; DAD scan: 210400 nm.

### Method 6:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 mi/min; temperature: 60° C.; DAD scan: 210-400 nm.

### LCMS

### Method 1:

Instrument: Waters Acquity UPLCMS Single Quad; column: BEH C 18 1.7 50×2.1 mm; Eluent A: water+0.05% formic acid (99%); Eluent B: acetonitrile+0.05% formic acid (99%); gradient: 0-0.2 2% B, 0.2-1.7 2-90% B, 1.7-1.9 90% B; 1.9-2.0 90-2% B, 2.0-2.5 2% B, flow 1.3 ml/min; temperature: 60° C.; DAD scan: 200-400 nm.

### Method 2:

Instrument: Agilent 1290 UPLCMS 6230 TOF; column: BEH C 18 1.7 μm, 50×2. mm; Eluent A: water+0.05% formic acid (99%); Eluent B: acetonitrile+0.05% formic acid (99/c); gradient: 0-1.7 2-90% B, 1.7-2.0 90% B; flow 1.2 ml/min; temperature: 60° C.; DAD scan: 190-400 nm.

### Intermediate 1a: tert-butyl 2-(5-fluoro-2-nitrophenyl)acetate

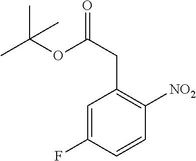 [SMILES: CC(C)(C)OC(=O)Cc1cc(F)ccc1[N+](=O)[O-]]

2-(5-Fluoro-2-nitrophenyl)acetic acid (10.1 g, 1 Eq, 50.7 mmol) was dissolved in tBuOH (130 mL), di-tert-butyl dicarbonate (10.5 g, 0.95 Eq, 48.2 mmol) followed by N,N-dimethylpyridin-4-amine (1.86 g, 0.3 Eq, 15.2 mmol) were added. Reaction was stirred 1 hour at room temperature (TLC (Hex EA 7:3): total conversion). The reaction was diluted with EtOAc and water/HCl 1N. The organic phase was washed with brine and dried over sodium sulfate. After filtration, the solvent was evaporated. The product (10.8 g, 42.3 mmol, 83.4%) was used in the next step without further purification.

¹H NMR (400 MHz, DMSO-d₆) δ ppm 8.16-8.27 (m, 1H), 7.47-7.52 (m, 1H), 7.40-7.47 (m, 1H), 3.99-4.04 (m, 2H), 1.34-1.41 (m, 9H).

UPLC-MS: R₄=1.22 min. MS (ESI−): m/z=254.4 [M−H]—.

### Intermediate 1b: tert-butyl 2-(5-fluoro-2-nitrophenyl)butanoate

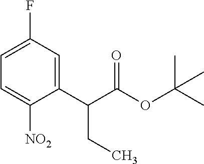 [SMILES: CCC(C(=O)OC(C)(C)C)c1cc(F)ccc1[N+](=O)[O-]]

Tert-butyl 2-(5-fluoro-2-nitrophenyl)acetate (500 mg, 1 Eq, 1.96 mmol) was dissolved in THF (15 mL), the resulting solution was cooled with and ice bath and NaH (86 mg, 60% Wt, 1.1 Eq, 2.15 mmol) was added at 0° C., the reaction was allowed to warm up to rt over 1h the cooled down again to 0° C. and iodoethane (336 mg, 173 μL, 1.1 Eq, 2.15 mmol) was added, the reaction was stirred for 16 hours the it was diluted with NH₄Cl saturated solution and EtOAc, the organic phase was washed with brined, dried over sodium sulfate and evaporated. The residue thus obtained was purified by automated flash column chromatography with a gradient 10-20-30% EtOAc in Hex to give 275 mg of desired material (0.97 mmol, 50%).

¹H NMR (400 MHz, DMSO-d₆) δ 8.09 (dd, J=8.9, 5.3 Hz, 1H), 7.47-7.39 (m, 2H), 3.90 (t, J=7.2 Hz, 1H), 2.09 (dd, J=14.1, 7.0 Hz, 1H), 1.85 (dt, J=13.8, 7.7 Hz, 1H), 1.33 (s, 10H), 0.85 (t, J=7.4 Hz, 3H).

### Intermediate 1c: tert-butyl 2-(5-fluoro-2-nitrophenyl)-2-methylpropanoate

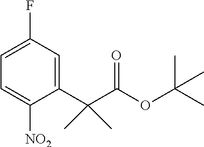 [SMILES: CC(C)(C)OC(=O)C(C)(C)c1cc(F)ccc1[N+](=O)[O-]]

Tert-butyl 2-(5-fluoro-2-nitrophenyl)acetate (200 mg, 1 Eq, 784 μmol) was dissolved in anhydrous DMF (3.0 mL), 18-crown-6 (31.1 mg, 0.15 Eq, 118 μmol) and iodomethane (745 mg, 327 μL, 6.7 Eq, 5.25 mmol) were added under inert atmosphere. The reaction was cooled with an ice bath and NaH (0.11 g, 60% Wt, 3.5 Eq, 2.8 mmol) was added, the reaction was stirred overnight. The reaction was diluted with EtOAc, HCl 1N, the organic phase was washed with brine, dried over sodium sulfate, and reduced in vacuo. The residue thus obtained was purified by automated flash column chromatography with a gradient 10-30-50% EtOAc in Hex to give 169 mg of desired material (0.78 mmol, 76%).

¹H NMR (400 MHz, DMSO-d₆) δ 8.07 (dd, J=8.9, 5.6 Hz, 1H), 7.60 (dd, J=10.9, 2.8 Hz, 1H), 7.39 (ddd, J=8.9, 7.3, 2.8 Hz, 1H), 1.57 (s, 6H), 1.31 (s, 9H)

### Intermediate 1d: tert-butyl 1-(5-fluoro-2-nitrophenyl)cyclopropane-1-carboxylate

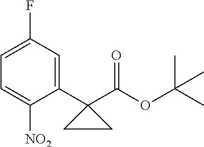 [SMILES: CC(C)(C)OC(=O)C1(c2cc(F)ccc2[N+](=O)[O-])CC1]

Tert-butyl 2-(5-fluoro-2-nitrophenyl)acetate (1000 mg, 1 Eq, 3.918 mmol) was dissolved in anhydrous DMF (7.5 mL), the solution was cooled with an ice bath and NaH (0.34 g, 60% Wt, 2.2 Eq, 8.619 mmol) was added, stirring was prolonged for 10 min. at rt, the reaction was then cooled again and 1,2-dibromoethane (809.6 mg, 371 μL, 1.1 Eq, 4.310 mmol) was added, the reaction was stirred at rt for 48 hours. The reaction was diluted with EtOAc and a saturated solution of NH₄Cl, the organic phase was washed with brine, dried over sodium sulfate, and reduced in vacuo. The residue thus obtained was purified by automated flash column chromatography with a gradient 10-30-50% EtOAc in Hex to give 115 mg of desired material (0.41 mmol, 10%).

¹H NMR (400 MHz, DMSO-d₆) δ 8.17 (dd, J=9.1, 5.3 Hz, 1H), 7.48 (dd, J=9.6, 2.8 Hz, 1H), 7.41 (ddd, J=9.0, 7.7, 2.8 Hz, 1H), 1.49 (br d, 0.1=3.3 Hz, 2H), 1.40 (d, 1=19.0 Hz, 1H), 1.35-1.29 (m, 1H), 1.27 (s, 9H).

### Intermediate 2: tert-butyl 2-(2-aminophenyl)acetate

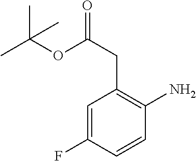 [SMILES: CC(C)(C)OC(=O)Cc1cc(F)ccc1N]

Tert-butyl 2-(5-fluoro-2-nitrophenyl)acetate (9.8 g, 1 Eq, 38 mmol) was dissolved in EtOAc (80 mL) under N₂ atmosphere. Palladium (10% on carbon) (0.82 g, 0.2 Eq, 7.7 mmol) was added and the resulting mixture was stirred at rt for 18 hours under H₂ atmosphere. The mixture was filtrated over celite, and the filtrate was evaporated to dryness. The product (8.6 g, 38 mmol, 99%) was used in the next step without further purification.

UPLC-MS: Rₜ=1.14 min. MS (ESI+): m/z=226.4 [M+H]⁺.

### Intermediate 2a: tert-butyl 2-(5-fluoro-2-(methylamino)phenyl)acetate

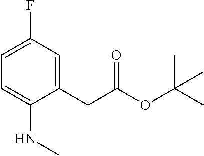 [SMILES: CNc1ccc(F)cc1CC(=O)OC(C)(C)C]

Intermediate 2 (685 mg, 1 Eq, 3.04 mmol) was dissolved in acetonitrile (10 mL). K₂CO (420 mg, 1 Eq, 3.04 mmol) and iodomethane (432 mg, 1 Eq, 3.04 mmol) were added and mixture was stirred at 50° C. for 6 hours. Acetonitrile was removed under vacuum, reaction mixture transferred to a separating funnel, EtOAc was added, and organic layer washed with water (2×). The obtained crude was purified on Biotage flash silica gel chromatography (Sfaer Silica 25 g, Hex/EtOAc=9/1). Product was obtained as yellow oil (147.9 mg, 18.9%).

¹H NMR (400 MHz, DMSO-d₆) δ: 6.89-6.96 (m, 1H), 6.85 (dd, J=9.63, 3.04 Hz, 1H), 6.45-6.51 (m, 1H), 4.90-4.98 (m, 1H), 3.46 (s, 2H), 2.67 (d, J=4.8 Hz, 3H), 1.39 (s, 9H).

UPLC-MS: Rₜ=1.27 min. MS (EST+): m/z=240.4 [M+H]⁺.

Intermediates 2b-f were prepared in analogy to intermediates 1a and 2 unless otherwise reported below.

| Int | name | structure | Analytical data |
|---|---|---|---|
| 2b | tert-butyl 2-(2- amino-3- fluorophenyl) acetate | 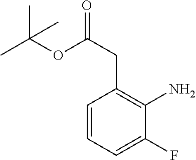 [SMILES: CC(C)(C)OC(=O)Cc1cccc(F)c1N] | UPLC-MS: Rₜ = 1.23 min. MS (ESI+): m/z = 226.4 [M + H]⁺. |
| 2c | tert-butyl 2-(2- amino-5- (trifluoromethyl) phenyl)acetate | 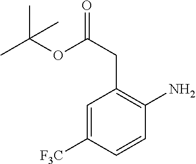 [SMILES: CC(C)(C)OC(=O)Cc1cc(C(F)(F)F)ccc1N] | ¹H NMR (400 MHz, DMSO-d₆) δ 7.29-7.21 (m, 2H), 6.73 (d, J = 7.9 Hz, 1H), 5.59 (s, 2H), 3.49 (s, 2H), 1.40 (s, 9H). |
| 2d | tert-butyl 2-(2- amino-5- chlorophenyl) acetate | 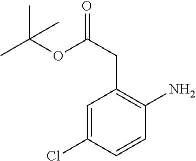 [SMILES: CC(C)(C)OC(=O)Cc1cc(Cl)ccc1N] | obtained as a 2.5:1 mixture of product and a byproduct. NMR data are given for the desired compound: ¹H NMR (400 MHz, DMSO-d₆) δ 7.00-6.95 (m, 2H), 6.66-6.60 (m, 1H), 5.03 (s, 2H), 3.42 (s, 2H), 1.40 (s, 9H). UPLC-MS (method 4): Rₜ = 1.24 min. MS (ESI+): m/z = 242.3 [M + H]⁺. |
| | tert-butyl 2-(2- amino-5- fluorophenyl) butanoate | 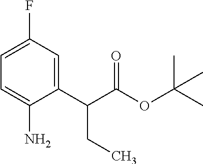 [SMILES: CCC(C(=O)OC(C)(C)C)c1cc(F)ccc1N] | ¹H NMR (400 MHz, DMSO-d₆) δ 6.85-6.71 (m, 2H), 6.70-6.61 (m, 1H), 4.86 (s, 2H), 3.58-3.47 (m, 1H), 1.89-1.75 (m, 1H), 1.66- 1.49 (m, 1H), 1.37 (s, 9H), 0.87 (t, J = 7.2 Hz, 3H). UPLC-MS: Rₜ = 1.28 min. MS (ESI+): m/z = 254.4 [M + H]⁺. |
| 2e | tert-butyl 2-(2- amino-5- fluorophenyl)-2- methylpropanoate | 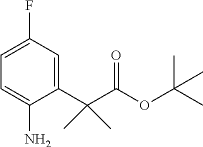 [SMILES: CC(C)(C)OC(=O)C(C)(C)c1cc(F)ccc1N] | obtained as a 1:1 mixture of product and a byproduct. NMR data are given for the mixture: ¹H NMR (400 MHz, DMSO-d₆) δ 10.33 (br s, 1H), 7.24 (dd, J = 8.5, 2.7 Hz, 1H), 6.97 (ddd, J = 9.8, 8.4, 2.8 Hz, 1H), 6.90 (dd, J = 11.0, 2.9 Hz, 1H), 6.84-6.76 (m, 2H), 6.66 (dd, J = 8.7, 5.4 Hz, 1H), 4.37 (s, 2H), 1.41 (s, 5H), 1.34 (s, 9H), 1.24 (s, 6H) UPLC-MS : Rₜ = 1.35 min. MS (ESI+): m/z = 254.4 [M + H]⁺. |
| 2f | tert-butyl 1-(2- amino-5- fluorophenyl) cyclopropane-1- carboxylate | 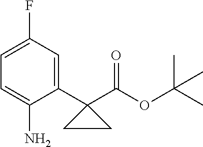 [SMILES: CC(C)(C)OC(=O)C1(c2cc(F)ccc2N)CC1] | UPLC-MS (method 6): Rₜ = 1.23 min. MS (ESI+): m/z = 252.5 [M + H]⁺. |

### Intermediate 2g: tert-butyl 2-(2-amino-3-chlorophenyl)acetate

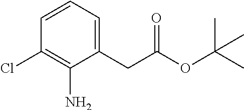 [SMILES: CC(C)(C)OC(=O)Cc1cccc(Cl)c1N]

tert-butyl 2-(3-chloro-2-nitrophenyl)acetate (300 mg, 1 Eq, 1.10 mmol) was dissolved in acetic acid (1.50 mL) and Ethanol (13.5 mL), zinc powder (722 mg, 10 Eq, 11.0 mmol) was added to the solution at 0° C. portion wise, the reaction mixture was stirred for 1 h at room temperature. The mixture was then filtered over a pad of celite and the solvent was removed in vacuo. The crude material thus obtained was purified by automated flash column chromatography with a gradient 10-30-50% EtOAc in Hex to give the desired material (246 mg, 92%).

¹H NMR (400 MHz, DMSO-d₆) δ 7.14 (dd, J=8.0, 1.4 Hz, 1H), 6.93 (dd, J=7.6, 1.5 Hz, 1H), 6.58-6.50 (m, 1H), 5.05 (s, 2H), 3.52 (s, 2H), 1.40 (s, 9H).

### Intermediate 3: 4-fluoro-3-nitrobenzoyl Chloride

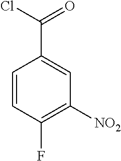 [SMILES: O=C(Cl)c1ccc(F)c([N+](=O)[O-])c1]

4-Fluoro-3-nitrobenzoic acid (7.3 g, 1 Eq, 39 mmol) was suspended in DCM (50 mL) under N₂ atmosphere. Oxalyl chloride (5.0 g, 3.5 mL, 1 Eq, 39 mmol) was added dropwise, followed by a catalytic amount of DMF. The resulting mixture was stirred at 25° C. for 18 hours. The mixture was evaporated to dryness. The product (8.0 g, 39 mmol, 100%) was used in the next step without further purification.

### Intermediate 4: tert-butyl 2-(5-fluoro-2-(4-fluoro-3-nitrobenzamido)phenyl)acetate

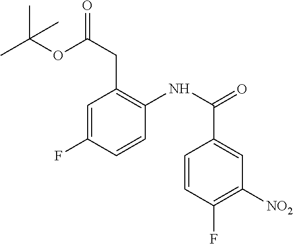 [SMILES: CC(C)(C)OC(=O)Cc1cc(F)ccc1NC(=O)c1ccc(F)c([N+](=O)[O-])c1]

4-Fluoro-3-nitrobenzoyl chloride (intermediate 3) (7.9 g, 1.02 Eq, 39 mmol) was dissolved in THF (60 mL) and NaHCO₃ (60 mL). Tert-butyl 2-(2-amino-5-fluorophenyl)acetate (intermediate 2) (8.6 g, 1 Eq, 38 mmol) was dissolved in THF (60 mL) and added to the reaction mixture dropwise. The resulting mixture was stirred at 25° C. for 10 min. Mixture is concentrated, H₂O was added, and the aqueous layer extracted with EtOAc (3×). The combined organics were evaporated to dryness and crude was purified by Biotage flash silica gel chromatography (SNAP Ultra 100 g, Hex/EtOAc=8/2 to 1/1), to give the title product (10.0 g, 25 mmol, 67%).

UPLC-MS: Rₜ=1.28 min. MS (ESI+): m/z=393.5 [M+H]⁺.

Intermediate 4a was prepared in analogy to intermediate 4.

| Int. | Name | Structure | Analytical Data |
|---|---|---|---|
| 4a | tert-butyl 2-(5-fluoro- 2-(4-fluoro-N- methyl-3- nitrobenzamido) phenyl)acetate | 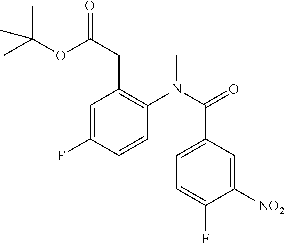 [SMILES: CN(C(=O)c1ccc(F)c([N+](=O)[O-])c1)c1ccc(F)cc1CC(=O)OC(C)(C)C] | ¹H NMR (400 MHz, DMSO-d₆) δ: 8.00-8.04 (m, 1 H), 7.57-7.63 (m, 1 H), 7.37-7.46 (m, 2 H), 7.06-7.16 (m, 2 H), 3.55 (s, 2 H), 3.24 (s, 3 H), 1.40 (s, 9 H). UPLC-MS: Rₜ = 1.31 min. MS (ESI+): m/z = 407.5 [M + H]⁺. |

### Intermediate 5: tert-butyl 2-(5-fluoro-2-(3-nitro-4-(piperidin-1-yl)benzamido)phenyl)acetate

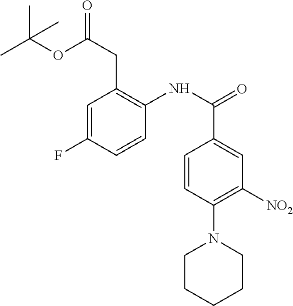 [SMILES: CC(C)(C)OC(=O)Cc1cc(F)ccc1NC(=O)c1ccc(N2CCCCC2)c([N+](=O)[O-])c1]

A mixture of tert-butyl 2-(5-fluoro-2-(4-fluoro-3-nitrobenzamido)phenyl)acetate (10 g, 1 Eq, 25 mmol), piperidine (2.6 g, 1.2 Eq, 31 mmol), triethylamine (3.1 g, 4.3 mL, 1.2 Eq, 31 mmol) and DMF (150 mL) was stirred at 25° C. for 18 hour. The mixture was evaporated to dryness. Crude was purified via Biotage flash silica gel chromatography (Sfaer Silica HC 200 g, Hex/EtOAc=10/0 to 3/7) to give the title product (5.1 g, 11 mmol, 44%).

UPLC-MS: Rₜ=1.49 min. MS (EST+): m/z=458.9 [M+H]⁺.

Intermediate 5a was prepared in analogy to intermediate 5.

| Int. | Name | Structure | Analytical Data |
|---|---|---|---|
| 5a | tert-butyl 2-(5-fluoro- 2-(N-methyl-3-nitro- 4-(piperidin-1- yl)benzamido)phenyl) acetate | 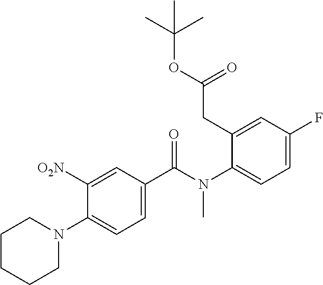 [SMILES: CN(C(=O)c1ccc(N2CCCCC2)c([N+](=O)[O-])c1)c1ccc(F)cc1CC(=O)OC(C)(C)C] | ¹H NMR (400 MHz, DMSO-d₆) δ: 7.61 (s, 1 H), 7.24-7.39 (m, 2 H), 7.07-7.22 (m, 2 H), 6.94-7.02 (m, 1 H), 3.43-3.59 (m, 2 H), 3.20 (s, 3 H), 2.89-2.99 (m, 4 H), 1.48-1.58 (m, 6 H), 1.38 (s, 9 H). UPLC-MS: Rₜ = 1.47 min. MS (ESI+): m/z = 473.0 [M + H]⁺. |

### Intermediate 6: tert-butyl 2-(2-(3-amino-4-(piperidin-1-yl)benzamido)-5-fluorophenyl)acetate

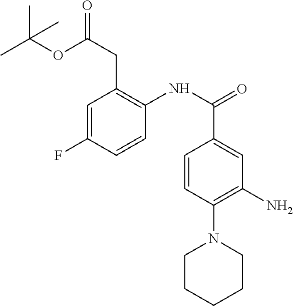 [SMILES: CC(C)(C)OC(=O)Cc1cc(F)ccc1NC(=O)c1ccc(N2CCCCC2)c(N)c1]

To a solution of tert-butyl 2-(5-fluoro-2-(3-nitro-4-(piperidin-1-yl)benzamido)phenyl)acetate (5.1 g, 1 Eq, 11 mmol) in EtOAc (200 mL) under N₂ atmosphere was added palladium (10% on carbon) (0.59 g, 0.05 Eq, 0.56 mmol). The resulting mixture was stirred at 25° C. for 18 hours under H₂ atmosphere. The mixture was filtrated over celite to give the title product (4.4 g, 9.9 mmol, 89%).

¹H NMR (400 MHz, DMSO-d₆) δ: 9.59 (s, 1H), 7.37 (dd, J=8.62, 5.58 Hz, 1H), 7.25 (d, J=2.03 Hz, 1H), 7.09-7.21 (m, 3H), 6.93 (dd, J=8.11 Hz, 1H), 4.85 (s, 2H), 3.63 (s, 2H), 2.75-2.84 (m, 4H), 1.63-1.72 (m, 4H), 1.49-1.58 (m, 2H), 1.28 (s, 9H).

UPLC-MS: Rₜ=1.44 min. MS (ESI+): m/z=429.0 [M+H]⁺.

Intermediate 6a was prepared in analogy to intermediate 6.

| Int. | Name | Structure | Analytical Data |
|---|---|---|---|
| 6a | tert-butyl 2-(2-(3- amino-N-methyl-4- (piperidin-1- yl)benzamido)-5- fluorophenyl)acetate | 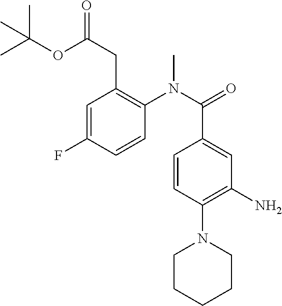 [SMILES: CN(C(=O)c1ccc(N2CCCCC2)c(N)c1)c1ccc(F)cc1CC(=O)OC(C)(C)C] | UPLC-MS: Rₜ = 1.44 min. MS (ESI+): m/z = 442.7 [M + H]⁺. |

### Intermediate 7a: methyl 1-(2,2-difluoroethyl)-1H-indazole-3-carboxylate

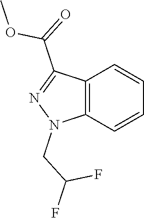 [SMILES: COC(=O)c1nn(CC(F)F)c2ccccc12]

Alkylation method described herein: Methyl 1H-indazole-3-carboxylate (300 mg, 1 Eq, 1.70 mmol) was dissolved in THF (3 mL) and the solution was cooled to 0° C. At this point sodium hydride (74.9 mg, 60% Wt, 1.1 Eq, 1.87 mmol) was added portion wise. The reaction was stirred at 0° C. for 10 min and then at 25° C. for 10 min. 2,2-difluoroethyl trifluoromethanesulfonate (438 mg, 272 μL, 1.2 Eq, 2.04 mmol) was added portion wise and the mixture was stirred for 3 h at 25° C. until complete conversion. The mixture was diluted with ethyl acetate and a saturated solution of NaHCO₃. The mixture was vigorously stirred for few minutes and then transferred into a separation funnel. The organic phase was washed with brine, dried over sodium sulfate, filtered and the solvent removed under reduce pressure. The remaining crude material was purified by prep-HPLC (Method described herein, Water (Basic): MeCN, gradient C) (309.2 mg of the title compound (1.287 mmol, 75.6%).

¹H NMR (500 MHz, DMSO-d₆) δ: 8.11 (m, 1H), 7.88 (d, 1H), 7.51-7.59 (m, 1H), 7.39 (m, 1H), 6.51 (m, 1H), 5.10 (m, 2H), 3.94 (s, 3H).

UPLC-MS: Rₜ=0.99 min. MS (EST+): m/z=241.4 [M+H]⁺.

Intermediate 7b: methyl 1-(3,3,3-trifluoropropyl)-1H-indazole-3-carboxylate

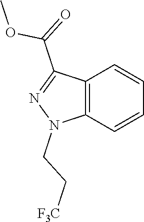 [SMILES: COC(=O)c1nn(CCC(F)(F)F)c2ccccc12]

Alkylation method 2: Methyl 1H-indazole-3-carboxylate (600 mg, 1 Eq, 3.41 mmol) was dissolved in DMF (6 mL) and the solution was cooled to 0° C. At this point sodium hydride (145 mg, 60% Wt, 1.1 Eq, 3.74 mmol) was added portion wise. The reaction was stirred at 0° C. for 10 min and then at 25° C. for 10 min. Cooled again in an ice-bath and 3-bromo-1,1,1-trifluoropropane (724 mg, 1.2 Eq, 4.09 mmol) in DMF (0.5 mL) was added. The mixture was stirred at 25° C. for 3 days. The mixture was diluted with ethyl acetate and a saturated solution of NaHCO₃. The mixture was vigorously stirred for few minutes and then transferred into a separation funnel. The organic phase was washed with brine, dried over sodium sulfate, filtered and the solvent removed under reduce pressure. The remaining crude material was purified by prep-HPLC (Method described herein, Water (Basic): MeCN, gradient D) to give 78 mg (8.4%) of the title compound.

¹H NMR (600 MHz, DMSO-d₆) &: 8.09 (m, 1H), 7.88 (d, 1H), 7.53 (m, 1H), 7.34-7.41 (m, 1H), 4.82 (m, 2H), 3.93 (s, 3H), 3.00 (qm, 2H).

UPLC-MS: Rₜ=1.10 min. MS (EST+): m/z=273.4 [M+H]⁺.

Intermediate 7c: Methyl 1-cyclopropyl-1H-indazole-3-carboxylate

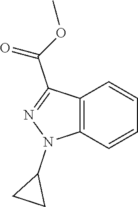 [SMILES: COC(=O)c1nn(C2CC2)c2ccccc12]

Alkylation method 3: Cyclopropylboronic acid (300 mg, 2 Eq, 3.49 mmol), methyl 1H-indazole-3-carboxylate (308 mg, 1 Eq, 1.75 mmol), sodium carbonate (370 mg, 2 Eq, 3.49 mmol) were dissolved in 1,2-dichloroethane (3 mL). Copper diacetate (317 mg, 1 Eq, 1.75 mmol) and 2,2′-bipyridine (273 mg, 1 Eq, 1.75 mmol) were added and the reaction mixture heated to 70° C. until complete conversion. The reaction mixture was quenched with water, extracted 3× times with DCM and concentrated under vacuum. The crude material was taken up with 10 ml 1N HCl, extracted with DCM, dried over a water repellent filter, and concentrated under vacuum. The remaining crude material was used in the next step without further purification.

UPLC-MS: Rₜ=1.06 min. MS (ESI+): n/z=217.4 [M+H]⁺.

### Intermediate 7d: methyl (S)-1-((tetrahydrofuran-2-yl)methyl)-1H-indazole-3-carboxylate

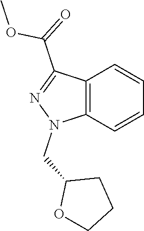 [SMILES: COC(=O)c1nn(C[C@@H]2CCCO2)c2ccccc12]

Alkylation method 4: In the first step, commercially available (S)-(tetrahydrofuran-2-yl)methanol (370 mg, 1 Eq, 3.62 mmol) was dissolved in DCM (5 mL) and triethylamine (733 mg, 1.01 mL, 2 Eq, 7.25 mmol) was added. The reaction mixture was cooled to 0° C., methanesulfonyl chloride (830 mg, 561 μL, 2 Eq, 7.25 mmol) was added and stirred at room temperature overnight. Mixture was evaporated to dryness. Fresh DCM was added and washed with water (2×) and brine (1×). Mixture is evaporated to dryness. In the second step, commercially available methyl 1H-indazole-3-carboxylate (300 mg, 1 Eq, 1.70 mmol) was dissolved in DMF (4 mL), cooled to 0° C., NaH (68 mg, 60% Wt, 1 Eq, 1.70 mmol) was added and the reaction mixture stirred at 0° C. for 15 min. Allowed to warm up to room temperature and stirred for 15 min. (S)-(tetrahydrofuran-2-yl)methyl methanesulfonate (307 mg, 1 Eq, 1.70 mmol) obtained in the first step was added at 0° C. to the reaction mixture and stirred for 5 days at 50° C. Reaction mixture was quenched with water, extracted 3× with DCM, organic phases combined and concentrated under vacuum. The obtained crude was purified by prep-HPLC (Method described herein, Water (Basic): MeCN, gradient C) to give 100 mg (22.6%) of the title compound.

¹H NMR (500 MHz, DMSO-d₆) δ: 8.07 (d, 1H), 7.83 (d, 1H), 7.48 (m, 1H), 7.34 (m, 1H), 4.51-4.64 (m, 2H), 4.254.34 (m, 1H), 3.93 (s, 3H), 3.64-3.73 (m, 1H), 3.54-3.62 (m, 11H), 1.93-2.04 (m, 1H), 1.77 (br d, 2H), 1.62-1.72 (m, 1H).

UPLC-MS (method 3): Rₜ=1.10 min. MS (ESI+): m/z=261.3 [M+H]⁺.

### Intermediate 7e: methyl 1-(1-cyclopropylethyl)-1H-indazole-3-carboxylate

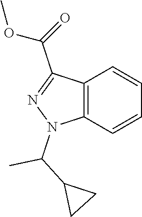 [SMILES: COC(=O)c1nn(C(C)C2CC2)c2ccccc12]

Alkylation method 5: Methyl 1H-indazole-3-carboxylate (1.9 g, 1 Eq, 11 mmol), 1-cyclopropylethan-1-ol (0.93 g, 1 Eq, 11 mmol) and Ph₃P (3.4 g, 1.2 Eq, 13 mmol) were dissolved in THE (5 mL). Cooled to 0° C., then di-tert-butyl (E)-diazene-1,2-dicarboxylate (3.2 g, 1.3 Eq, 14 mmol) was added dropwise. Mixture is stirred 5 minutes at 0° C., then 2 hours at 25° C. EtOAc and water were added, and the aqueous layer was extracted with EtOAc (3×). The combined organics were evaporated to dryness and purified by prep-HPLC (Method described herein, Water (Acid): MeCN, gradient D) to give the title compound (300 mg, 11%).

¹H NMR (400 MHz, DMSO-d₆) &: 8.06-8.11 (m, 1H), 7.83-7.89 (m, 1H), 7.43-7.50 (m, 1H), 7.30-7.37 (m, 1H), 4.33 (dq, J=9.25, 6.72 Hz, 1H), 3.92 (s, 3H), 1.62 (d, J=6.84 Hz, 3H), 1.37-1.48 (m, 1H), 0.58-0.67 (m, 1H), 0.41-0.49 (m, 1H), 0.31-0.39 (m, 1H), 0.22-0.30 (m, 1H).

UPLC-MS: Rₜ=1.23 min. MS (ESI+): m/z=245.3 [M+H]⁺.

### Intermediate 7f: Methyl 1-(2-hydroxy-2-methylpropyl)-1H-indazole-3-carboxylate

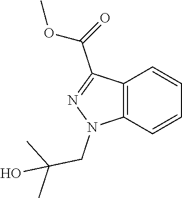 [SMILES: COC(=O)c1nn(CC(C)(C)O)c2ccccc12]

Alkylation method 6: Methyl 1H-indazole-3-carboxylate (250 mg, 1 eq, 1.42 mmol) and caesium carbonate (555 mg, 1.2 eq, 1.70 mmol) were taken up in MeCN (2 mL). At this point 2,2-dimethyloxirane (512 mg, 633 μL, 5 eq, 7.10 mmol) was added portion wise. The reaction was stirred at 80° C. until complete conversion. The mixture was diluted with ethyl acetate and a saturated aqueous solution of NaHCO₃. The mixture was vigorously stirred for several minutes and then transferred into a separation funnel. The organic phase was washed with brine, dried over sodium sulfate, filtered and the solvent removed under reduce pressure. The remaining crude was purified by prep-HPLC (Method described herein, acidic eluent, gradient C) to give 139 mg (0.56 mmol) of the title compound.

¹H NMR (600 MHz, DMSO-d₆) δ: 8.03-8.10 (m, 1H), 7.85 (d, 1H), 7.44-7.46 (m, 1H), 7.46 (m, 2H), 7.28-7.35 (m, 1H), 7.28-7.29 (m, 1H), 4.76 (s, 1H), 4.43 (s, 2H), 3.93 (s, 3H), 1.14 (s, 6H).

UPLC-MS: Rₜ=0.91 min. MS (ESI+): m/z=249.5 [M+H]⁺.

Intermediates 7g-7ap were prepared in analogy to intermediates 7a-f using either THF or DMF as solvent.

| | | Structure/ | |
|---|---|---|---|
| Int. | Name | Alkylation method | Analytical Data |
| 7g | methyl 1-(2-((tert- butoxycarbonyl) amino)ethyl)-1H- indazole-3- carboxylate | 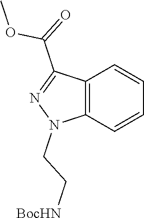 [SMILES: COC(=O)c1nn(CCNC(=O)OC(C)(C)C)c2ccccc12] | ¹H NMR (600 MHz, DMSO-d6) δ: 8.07 (d, 1H), 7.71 (d, 1H), 7.44-7.54 (m, 1H), 7.33 (m, 1H), 6.48-6.97 (m, 1H), 4.48- 4.61 (m, 2H), 3.92 (s, 3H), 3.36-3.44 (m, 2H), 1.25 (s, 9H). UPLC-MS: Rₜ = 1.13 min. MS (ESI+): m/z = 320.5 [M + H]⁺. |
| 7h | methyl 1-((1-(tert- butoxycarbonyl) piperidin-4- yl)methyl)-1H- indazole-3- carboxylate | 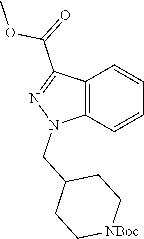 [SMILES: COC(=O)c1nn(CC2CCN(C(=O)OC(C)(C)C)CC2)c2ccccc12] | ¹H NMR (600 MHz, DMSO-d6) δ: 8.08 (d, 1H), 7.88 (d, 1H), 7.50 (m, 1H), 7.32-7.38 (m, 1H), 4.44 (d, 2H), 3.90- 3.94 (m, 2H), 3.92 (s, 3H), 2.54-2.75 (m, 2H), 2.13 (tm, 1H), 1.39-1.47 (m, 2H), 1.37 (s, 9H), 1.16 (qd, 2H). UPLC-MS: Rₜ = 1.31 min. MS (ESI+): m/z = 374.6 [M + H]⁺. prep-HPLC (Method described herein, Water (Basic): MeCN, gradient D) |
| 7i | methyl 1-(2,2,2- trifluoroethyl)- 1H-pyrazolo[4,3- c]pyridine-3- carboxylate | 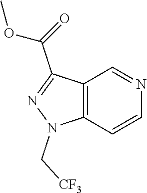 [SMILES: COC(=O)c1nn(CC(F)(F)F)c2ccncc12] | ¹H NMR (500 MHz, DMSO-d6) δ: 9.41 (d, 1H), 8.61 (d, 1H), 7.98 (m, 1H), 5.71 (m, 2H), 3.99 (s, 3H). UPLC-MS: Rₜ = 0.82 min. MS (ESI+): m/z = 260.4 [M + H]⁺. prep-HPLC (Method described herein, Water (Basic): MeCN, gradient C) |
| | | Method described | |
| | | herein | |
| 7j | ethyl 1-(2,2,2- trifluoroethyl)- 1H-pyrazolo[4,3- b]pyridine-3- carboxylate | 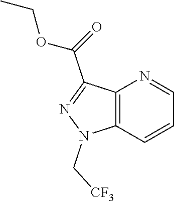 [SMILES: CCOC(=O)c1nn(CC(F)(F)F)c2cccnc12] | ¹H NMR (500 MHz, DMSO-d6) δ: 8.79 (m, 1H), 8.42 (m, 1H), 7.62 (m, 1H), 5.71 (m, 2H), 4.43 (m, 2H), 1.37 (m, 3H). UPLC-MS: Rₜ = 0.85 min. MS (ESI+): m/z = 274.4 [M + H]⁺. |
| | | Method described | |
| | | herein | |
| 7k | ethyl 1-(2,2,2- trifluoroethyl)- 1H-pyrazolo[3,4- c]pyridine-3- carboxylate | 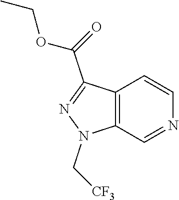 [SMILES: CCOC(=O)c1nn(CC(F)(F)F)c2cnccc12] | ¹H NMR (600 MHz, DMSO-d6) δ: 9.44 (s, 1H), 8.52 (d, 1H), 8.03 (dd, 1H), 5.83 (q, 2H), 4.45 (q, 2H), 1.40 (t, 3H). UPLC-MS: Rₜ = 0.96 min. MS (ESI+): m/z = 274.4 [M + H]⁺. prep-HPLC (Method described herein, Water (Basic): MeCN, gradient C) |
| 7l | methyl 1-(2,2,2- trifluoroethyl)- 1H-pyrazolo[3,4- b]pyridine-3- carboxylate | 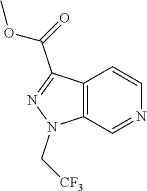 [SMILES: COC(=O)c1nn(CC(F)(F)F)c2cnccc12] | ¹H NMR (600 MHz, DMSO-d6) δ: 8.76 (dd, 1H), 8.55 (dd, 1H), 7.54 (dd, 1H), 5.58 (q, 2H), 3.98 (s, 3H). UPLC-MS (method 4): Rₜ = 1.01 min. MS (ESI+): m/z = 260.3 [M + H]⁺. prep-HPLC (Method described herein, Water (Acidic): MeCN, gradient C) |
| 7m | methyl 1-(2- hydroxyethyl)- 1H-indazole-3- carboxylate | 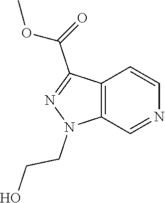 [SMILES: COC(=O)c1nn(CCO)c2cnccc12] | 1H NMR (500 MHz, DMSO-d6) δ: 8.02-8.12 (m, 1H), 7.81 (d, 1H), 7.48 (m, 1H), 7.30-7.38 (m, 1H), 4.91 (m, 1H), 4.56 (m, 2H), 3.92 (s, 3H), 3.85 (m, 2H). UPLC-MS: Rₜ = 0.78 min. MS (ESI+): m/z = 221.4 [M + H]⁺. |
| 7n | methyl 1-(2- methoxyethyl)- 1H-indazole-3- carboxylate | 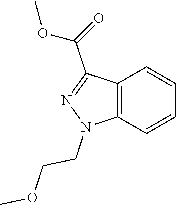 [SMILES: COCCn1nc(C(=O)OC)c2ccccc21] | UPLC-MS: Rₜ = 0.96 min. MS (ESI+): m/z = 235.6 [M + H]⁺. prep-HPLC (Method described herein, Water (Basic): MeCN, gradient C) |
| 7o | ethyl 4-phenyl-1- (2,2,2- trifluoroethyl)- 1H-pyrazole-3- carboxylate | 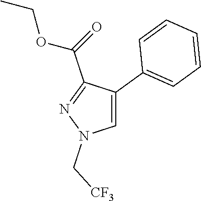 [SMILES: CCOC(=O)c1nn(CC(F)(F)F)cc1-c1ccccc1] | ¹H NMR (500 MHz, DMSO-d6) δ: 8.15 (s, 1H), 7.42-7.47 (m, 2H), 7.36-7.41 (m, 2H), 7.30-7.35 (m, 1H), 5.30 (m, 2H), 4.23 (m, 2H), 1.20 (m, 3H). UPLC-MS: Rₜ = 1.17 min. MS (ESI+): m/z = 299.5 [M + H]⁺. prep-HPLC (Method described herein, Water (Basic): MeCN, gradient D) |
| 7p | ethyl 5-phenyl-1- (2,2,2- trifluoroethyl)- 1H-pyrazole-3- carboxylate | 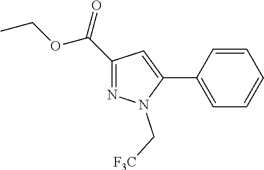 [SMILES: CCOC(=O)c1cc(-c2ccccc2)n(CC(F)(F)F)n1] | ¹H NMR (500 MHz, DMSO-d6) 6: 7.54 (s, 5H), 6.95 (s, 1H), 5.19 (m, 2H), 4.32 (m, 2H), 1.32 (m, 3H). UPLC-MS: Rₜ = 1.21 min. MS (ESI+): m/z = 299.4 [M + H]⁺. prep-HPLC (Method described herein, Water (Basic): MeCN, gradient D) |
| 7q | methyl 4-bromo- 1-(2,2,2- trifluoroethyl)- 1H-pyrazole-3- carboxylate | 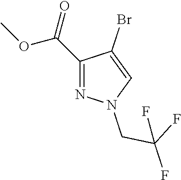 | ¹H NMR (500 MHz, DMSO-d6) δ: 8.26- 8.30 (m, 1H), 5.28 (m, 2H), 3.83 (s, 3H). |
| 7r | methyl 1- ((tetrahydrofuran- 3-yl)methyl)-1H- indazole-3- carboxylate | 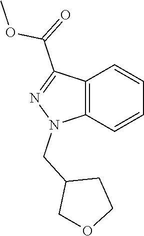 [SMILES: COC(=O)c1nn(CC2CCOC2)c2ccccc12] | ¹H NMR (500 MHz, DMSO-d6) δ: 8.09 (m, 1H), 7.85-7.92 (m, 1H), 7.51 (m, 1H), 7.36 (m, 1H), 4.53 (d, 2H), 3.93 (s, 3H), 3.82 (m, 1H), 3.60-3.69 (m, 2H), 3.50 (m, 1H), 2.79-2.92 (m, 1H), 1.84- 1.96 (m, 1H), 1.60-1.71 (m, 1H). UPLC-MS: Rₜ = 0.97 min. MS (ESI+): m/z = 261.5 [M + H]⁺. prep-HPLC (Method described herein, Water (Basic): MeCN, gradient C) |
| 7s | methyl 1- ((tetrahydrofuran- 2-yl)methyl)-1H- indazole-3- carboxylate | 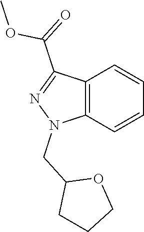 [SMILES: COC(=O)c1nn(CC2CCCO2)c2ccccc12] | ¹H NMR (600 MHz, DMSO-d6) δ: 8.05- 8.10 (m, 1H), 7.81-7.86 (m, 1H), 7.49 (m, 1H), 7.34 (m, 1H), 4.53-4.62 (m, 2H), 4.29 (qd, 1H), 3.93 (s, 3H), 3.68 (m, 1H), 3.55-3.62 (m, 1H), 1.94-2.04 (m, 1H), 1.71-1.83 (m, 2H), 1.64-1.71 (m, IH). UPLC-MS: Rₜ = 1.03 min. MS (ESI+): m/z = 261.6 [M + H]⁺. prep-HPLC (Method described herein, Water (Basic): MeCN, gradient C) |
| 7t | methyl 1-(prop-2- yn-1-yl)-1H- indazole-3- carboxylate | 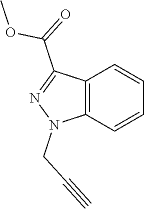 [SMILES: C#CCn1nc(C(=O)OC)c2ccccc21] | ¹H NMR (600 MHz, DMSO-d6) δ: 8.11 (d, 1H), 7.87 (d, 1H), 7.55 (m, 1H), 7.36-7.42 (m, 1H), 5.50 (d, 2H), 3.94 (s, 3H), 3.50 (m, 1H). UPLC-MS: Rₜ = 1.02 min. MS (ESI+): m/z = 215.4 [M + H]⁺. prep-HPLC (Method described herein, Water (Acidic): MeCN, gradient D) |
| 7u | methyl 1-(but-2- yn-1-yl)-1H- indazole-3- carboxylate | 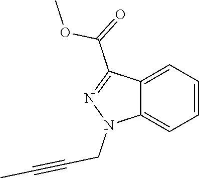 [SMILES: CC#CCn1nc(C(=O)OC)c2ccccc21] | UPLC-MS: Rₜ = 1.12 min. MS (ESI+): m/z = 229.3 [M + H]⁺. |
| 7v | methyl 1-((5- methylisoxazol-3- yl)methyl)-1H- indazole-3- carboxylate | 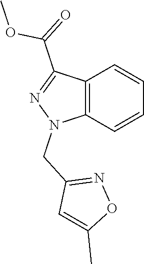 [SMILES: COC(=O)c1nn(Cc2cc(C)on2)c2ccccc12] | ¹H NMR (500 MHz, DMSO-d6) δ: 8.10 (d, 1H), 7.86 (d, 1H), 7.49-7.58 (m, 1H), 7.35-7.42 (m, 1H), 6.06 (m, 1H), 5.83- 5.89 (m, 2H), 3.93 (s, 3H), 2.33 (d, 3H). UPLC-MS: Rₜ = 1.02 min. MS (ESI+): m/z = 272.4 [M + H]⁺. prep-HPLC (Method described herein, Water (Basic): MeCN, gradient C) |
| 7w | methyl 1-((5- methyl-1,2,4- oxadiazol-3- yl)methyl)- 1H- indazole-3- carboxylate | 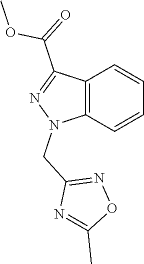 [SMILES: COC(=O)c1nn(Cc2noc(C)n2)c2ccccc12] | ¹H NMR (500 MHz, DMSO-d6) δ: 8.10 (m, 1H), 7.85-7.91 (m, 1H), 7.54 (m, 1H), 7.39 (m, 1H), 5.99 (s, 2H), 3.93 (s, 3H), 2.53 (s, 3H). UPLC-MS: Rₜ = 0.94 min. MS (ESI+): m/z = 273.4 [M + H]⁺. prep-HPLC (Method described herein, Water (Basic): MeCN, gradient C) |
| 7x | methyl 1-(2,2,2- trifluoroethyl)- 1H-indole-3- carboxylate | 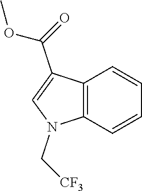 [SMILES: COC(=O)c1cn(CC(F)(F)F)c2ccccc12] | ¹H NMR (400 MHz, DMSO-d6) δ: 8.20 (s, 1 H), 8.02 − 8.07 (m, 1 H), 7.68 − 7.77 (m, 1 H), 7.24 − 7.38 (m, 2 H), 5.35 (q, J = 9.12 Hz, 2 H), 3.83 (s, 3 H). UPLC-MS: Rₜ = 1.14 min. MS (ESI+): m/z = 258.4 [M + H]⁺. |
| 7y | methyl 2-methyl- 1-(2,2,2- trifluoroethyl)- 1H-indole-3- carboxylate | 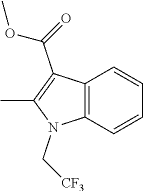 [SMILES: COC(=O)c1c(C)n(CC(F)(F)F)c2ccccc12] | ¹H NMR (400 MHz, DMSO-d6) δ: 7.94 − 8.02 (m, 1 H), 7.62 − 7.70 (m, 1 H), 7.18 − 7.29 (m, 2 H), 5.28 − 5.40 (m, 2 H), 3.85 (s, 3 H), 2.75 (s, 3 H). UPLC-MS: Rₜ = 1.22 min. MS (ESI+): m/z = 272.4 [M + H]⁺. |
| 7z | methyl 5-methyl- 1-(2,2,2- trifluoroethyl)- 1H-indazole-3- carboxylate | 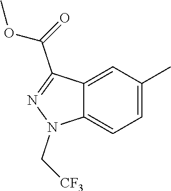 | UPLC-MS: Rₜ = 1.20 min. MS (ESI+): m/z = 273.3 [M + H]⁺. |
| 7aa | methyl 5-fluoro- 1-(2,2,2- trifluoroethyl)- 1H-indazole-3- carboxylate | 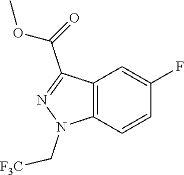 [SMILES: COC(=O)c1nn(CC(F)(F)F)c2ccc(F)cc12] | ¹H NMR (500 MHz, DMSO-d6) δ: 8.02 (m, 1H), 7.78 (m, 1H), 7.53 (m, 1H), 5.68 (m, 2H), 3.95 (s, 3H). UPLC-MS: Rₜ = 1.12 min. MS (ESI+): m/z = 277.3 [M + H]⁺. prep-HPLC (Method described herein, Water (Basic): MeCN, gradient D) |
| 7ab | methyl 1-((1,4- dioxan-2- yl)methyl)-1H- indazole-3- carboxylate | 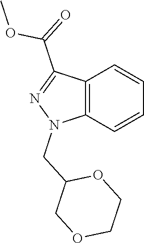 [SMILES: COC(=O)c1nn(CC2COCCO2)c2ccccc12] | ¹H NMR (500 MHz, DMSO-d6) δ: 8.04- 8.10 (m, 1H), 7.83 (d, 1H), 7.49 (m, 1H), 7.35 (m, 1H), 4.52-4.64 (m, 2H), 3.96-4.03 (m, 1H), 3.93 (s, 3H), 3.83 (m, 1H), 3.56-3.71 (m, 2H), 3.38-3.52 (m, 2H), 3.31-3.37 (m, 1H). UPLC-MS: Rₜ = 0.95 min. MS (ESI+): m/z = 277.4 [M + H]⁺. prep-HPLC (Method described herein, Water (Basic): MeCN, gradient C) |
| 7ac | methyl 5- methoxy-1-(2,2,2- trifluoroethyl)- 1H-indazole-3- carboxylate | 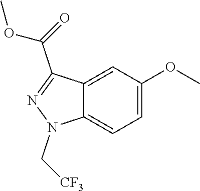 | UPLC-MS (method 6): Rₜ = 1.11 min. MS (ESI+): m/z = 289.5 [M + H]⁺. |
| 7ad | methyl 1- ((tetrahydro-2H- pyran-3- yl)methyl)-1H- indazole-3- carboxylate | 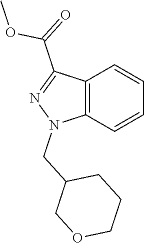 [SMILES: COC(=O)c1nn(CC2CCCOC2)c2ccccc12] | ¹H NMR (600 MHz, DMSO-d6) δ: 8.04- 8.12 (m, 1H), 7.86 (d, 1H), 7.51 (m, 1H), 7.32-7.39 (m, 1H), 4.46-4.51 (m, 1H), 4.38-4.45 (m, 1H), 3.93 (s, 3H), 3.66- 3.74 (m, 1H), 3.61 (m, 1H), 3.30-3.39 (m, 1H), 3.22 (m, 1H), 2.13-2.24 (m, 1H), 1.55-1.69 (m, 2H), 1.39-1.50 (m, 1H), 1,23-1.34 (m, 1H), UPLC-MS: Rₜ = 1.07 min. MS (ESI+): m/z = 275.4 [M + H]⁺. prep-HPLC (Method described herein, Water (Basic): MeCN, gradient C) |
| 7ae | methyl 1-(1,1,1- trifluoropropan-2- yl)-1H-indazole- 3-carboxylate | 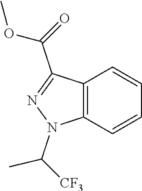 [SMILES: COC(=O)c1nn(C(C)C(F)(F)F)c2ccccc12] | UPLC-MS (method 4): Rₜ = 1.20 min. MS (ESI+): m/z = 273.3 [M + H]⁺. prep-HPLC (Method described herein, Water (Acid): MeCN, gradient D) |
| | | Method described | |
| | | herein | |
| 7af | methyl 4-methyl- 1-(2,2,2- trifluoroethyl)- 1H-indazole-3- carboxylate | 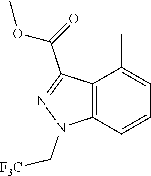 [SMILES: COC(=O)c1nn(CC(F)(F)F)c2cccc(C)c12] | ¹H NMR (500 MHz, DMSO-d6) δ: 7.74 (d, 1H), 7.44 (m, 1H), 7.14 (d, 1H), 5.60 (m, 2H), 3.92 (s, 3H), 2.71 (s, 3H). UPLC-MS: Rₜ = 1.20 min. MS (ESI+): m/z = 273.3 [M + H]⁺. prep-HPLC (Method described herein, Water (Acid): MeCN, gradient C) |
| 7ag | methyl (R)-1- ((tetrahydrofuran- 2-yl)methyl)-1H- indazole-3- carboxylate | 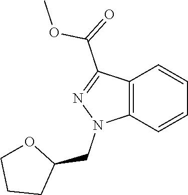 [SMILES: COC(=O)c1nn(C[C@H]2CCCO2)c2ccccc12] | ¹H NMR (500 MHz, DMSO-d6) δ: 8.03- 8.11 (m, 1H), 7.83 (d, 1H), 7.48 (m, 1H), 7.34 (m, 1H), 4.52-4.63 (m, 2H), 4.26-4.34 (m, 1H), 3.93 (s, 3H), 3.64-3.74 (m, 1H), 3.54-3.61 (m, 1H), 1.93-2.04 (m, 1H), 1.72- 1.82 (m, 2H), 1.63-1.71 (m, 1H). UPLC-MS (method 6): Rₜ = 1.05 min. MS (ESI+): m/z = 261.5 [M + H]⁺. prep-HPLC (Method described herein, Water (Acid): MeCN, gradient C) |
| 7ah | methyl 6-methyl- 1-(2,2,2- trifluoroethyl)- 1H-indazole-3- carboxylate | 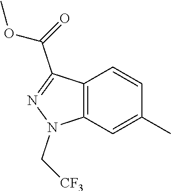 | ¹H NMR (500 MHz, DMSO-d6) δ: 7.99 (d, 1H), 7.71-7.76 (m, 1H), 7.25 (m, 1H), 5.58 (m, 2H), 3.94 (s, 3H), 2.50 (s, 3H). UPLC-MS: Rₜ = 1.16 min. MS (ESI+): m/z = 273.3 [M + H]⁺. prep-HPLC (Method described herein, Water (Basic): MeCN, gradient D) |
| 7ai | methyl 1-(2,2,2- trifluoroethyl)- 1,4,5,6- tetrahydrocyclopenta [c]pyrazole-3- carboxylate | 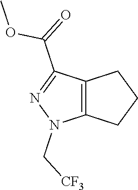 [SMILES: COC(=O)c1nn(CC(F)(F)F)c2c1CCC2] | ¹H NMR (500 MHz, DMSO-d6) δ: 5.15 (m, 2H), 3.77 (s, 3H), 2.73-2.79 (m, 2H), 2.66- 2.72 (m, 2H), 2.52-2.55 (m, 2H). UPLC-MS (method 5): Rₜ = 1.03 min. MS (ESI+): m/z = 249.5 [M + H]⁺. prep-HPLC (Method described herein, Water (Basic): MeCN, gradient C) |
| 7aj | methyl 1-(1- (tetrahydro-2H- pyran-4-yl)ethyl)- 1H-indazole-3- carboxylate | 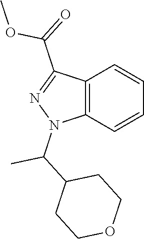 [SMILES: COC(=O)c1nn(C(C)C2CCOCC2)c2ccccc12] | ¹H NMR (600 MHz, DMSO-d6) δ: 8.05- 8.13 (m, 1H), 7.92 (d, 1H), 7.48 (m, 1H), 7.31-7.37 (m, 1H), 4.72-4.80 (m, 1H), 3.92 (s, 3H), 3.88 (br s, 1H), 3.67 (m, 1H), 3.29 (d, 1H), 3.11 (m, 1H), 2.07-2.19 (m, 1H), 1.74 (br m, 1H), 1.52 (d, 3H), 1.35 (qd, 1H), 1.15 (qd, 1H), 0.68-0.78 (m, 1H). UPLC-MS: Rₜ = 1.10 min. MS (ESI+): m/z = 289.3 [M + H]⁺. |
| 7ak | ethyl 3-phenyl-1- (2,2,2- trifluoroethyl)- 1H-pyrazole-5- carboxylate | 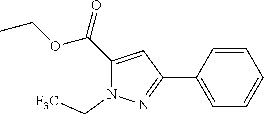 [SMILES: CCOC(=O)c1cc(-c2ccccc2)nn1CC(F)(F)F] | ¹H-NMR (600 MHz, DMSO-d6): δ 7.88 − 7.92 (m, 2H), 7.55 (s, 1H), 7.42 − 7.48 (m, 2H), 7.37 − 7.41 (m, IH), 5.51 (q, 2H), 4.37 (q, 2H), 1.35 (t, 3H). UPLC-MS: Rₜ = 1.38 min. MS (ESI+): m/z = 299.4 [M + H]⁺. |
| | | Method described | |
| | | herein | |
| 7al | methyl 2-(2,2,2- trifluoroethyl)- 2H-indazole-3- carboxylate | 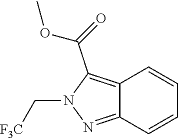 [SMILES: COC(=O)c1c2ccccc2nn1CC(F)(F)F] | ¹H-NMR (500 MHz, DMSO-d6) δ: 8.03 (dt, 1H), 7.86 (dt, 1H), 7.44 − 7.51 (m, 1H), 7.37 − 7.43 (m, 1H), 5.86 (q, 2H), 4.02 (s, 3H). UPLC-MS: Rₜ = 1.18 min. MS (ESI+): m/z = 259.3 [M + H]⁺. prep-HPLC (Method described herein, Water (Basic): MeCN, gradient D) |
| 7am | methyl 1- (cyclopropylmethyl)- 1H-indazole- 3-carboxylate | 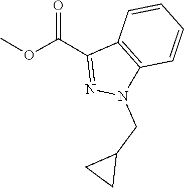 [SMILES: COC(=O)c1nn(CC2CC2)c2ccccc12] | ¹H-NMR (600 MHz, DMSO-d₆): δ [ppm]= 0.41 − 0.54 (m, 4H), 1.27 − 1.40 (m, 1H), 3.93 (s, 3H), 4.43 (d, 2H), 7.30 − 7.38 (m, 1H), 7.49 (ddd, 1H), 7.87 (d, 1H), 8.09 (d, 1H). |
| 7an | methyl 1- isobutyl-1H- indazole-3- carboxylate | 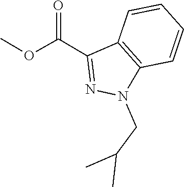 [SMILES: COC(=O)c1nn(CC(C)C)c2ccccc12] | UPLC-MS (method 5): Rₜ = 1.23 min. MS (ESI+): m/z = 232.3 [M + H]⁺. |
| 7ao | methyl 1- (cyclobutylmethyl)- 1H-indazole-3- carboxylate | 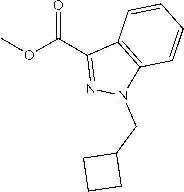 [SMILES: COC(=O)c1nn(CC2CCC2)c2ccccc12] | LC-MS: Rₜ = 1.28 min. MS (ESI+): m/z = 245.5 [M + H]⁺. |
| 7ap | methyl 1- ((tetrahydro-2H- pyran-4- yl)methyl)-1H- indazole-3- carboxylate | 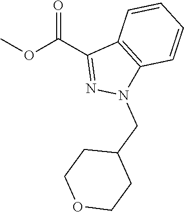 [SMILES: COC(=O)c1nn(CC2CCOCC2)c2ccccc12] | ¹H NMR (400 MHz, DMSO-d₆) δ 8.08 (dt, J = 8.1, 1.0 Hz, 1H), 7.93 − 7.84 (m, 1H), 7.50 (ddd, J = 8.4, 7.0, 1.0 Hz, 1H), 7.34 (ddd, J = 8.0, 7.0, 0.9 Hz, 1H), 4.43 (d, J = 7.1 Hz, 2H), 3.92 (s, 3H), 3.85 − 3.73 (m, 2H), 3.21 (td, J = 11.0, 3.7 Hz, 2H), 2.27 − 2.13 (m, 1H), 1.38 − 1.27 (m, 4H) UPLC-MS (method 5): Rₜ = 1.04 min. MS (ESI+): m/z = 275.6 [M + H]⁺. |

### Intermediate 8a: 1-(2,2-Difluoroethyl)-1H-indazole-3-carboxylic acid

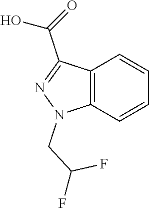 [SMILES: O=C(O)c1nn(CC(F)F)c2ccccc12]

Saponification method described herein: Intermediate 7a (300 mg, 1 Eq, 1.25 mmol) was dissolved in THF (4 mL) and then LiOH (120 mg, 5.00 mL, 1 molar, 4 Eq, 5.00 mmol) was added portion wise. The mixture was stirred at 25° C. for 2 h. The organic solvent was removed under reduce pressure. 1 M HCl solution was added, and the pH adjusted to pH: 1. Ethyl acetate was added, and the aqueous phase was extracted with ethyl acetate. The organic phase was washed successively with 1 M HCl and brine. Aqueous phases were combined and washed with DCM. Organic phases were combined, dried on sodium sulfate, filtered and the solvent removed under reduce pressure to give 259.6 mg (1.148 mmol) of the title compound.

UPLC-MS: Rₜ=0.84 min. MS (ESI+): m/z=227.4 [M+H]⁺.

### Intermediate 8b: 1-(3,3,3-trifluoropropyl)-1H-indazole-3-carboxylic acid

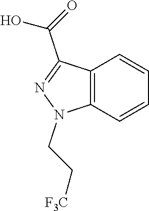 [SMILES: O=C(O)c1nn(CCC(F)(F)F)c2ccccc12]

Saponircation method 2S Intermediate 7b (75 mg, 1 Eq 0.28 mmol) was dissolved in THF (2 mL) and LiOH (33 mg, 1.4 mL, 1 molar, 5 Eq, 1.4 mmol) was added. The mixture was stirred at 25° C. for 1 h. The organic solvent was removed under reduce pressure. 1 M HCl solution was added, and the pH adjusted to pH: 1. White precipitate was formed, filtered, and washed with water and hexane. Dried under high vacuum overnight to give the title compound (60.3 mg, 85%).

UPLC-MS: Rₜ=0.96 min. MS (ESI+): m/z=259.3 [M+H]⁺.

Intermediates 8c-ao were prepared in analogy to intermediates 8a and 8b using THF or MeOH as a solvent, at room temperature or heating the reaction mixture to 80° C.

| | | Structure/ | |
|---|---|---|---|
| | | Saponification | |
| Int. | Name | method | Analytical Data |
| 8c | 1-cyclopropyl-1H- indazole-3- carboxylic acid | 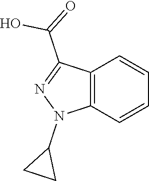 [SMILES: O=C(O)c1nn(C2CC2)c2ccccc12] | UPLC-MS: Rₜ = 0.45 min. MS (ESI+): m/z =203.4 [M + H]⁺. |
| 8d | 1-(2-((tert- butoxycarbonyl) amino)ethyl)-1H- indazole-3- carboxylic acid | 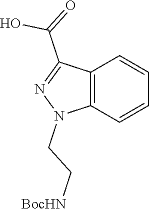 [SMILES: CC(C)(C)OC(=O)NCCn1nc(C(=O)O)c2ccccc21] | ¹H NMR (400 MHz, DMSO-d6) δ: 12.98 (br s, 1H), 8.07 (d, J = 8.12 Hz, 1H), 7.68 (d, J = 8.60 Hz, 1H), 7.43- 7.49 (m, 1H), 7.26-7.34 (m, 1H), 6.88- 6.97 (m, 1H), 4.48-4.56 (m, 2H), 3.30- 3.41 (m, 2H), 1.25 (s, 9H). UPLC-MS: Rₜ = 0.96 min. MS (ESI+): m/z = 306.5 [M + H]⁺. |
| 8e | 1-((1-(tert- butoxycarbonyl) piperidin-4-yl)methyl)- 1H-indazole-3- carboxylic acid | 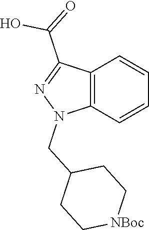 [SMILES: CC(C)(C)OC(=O)N1CCC(Cn2nc(C(=O)O)c3ccccc32)CC1] | UPLC-MS: Rₜ = 0.67 min. MS (ESI−): m/z = 358.5 [M − H]⁻. |
| 8f | 1-(2,2,2- trifluoroethyl)-1H- pyrazolo[4,3- c]pyridine-3- carboxylic acid | 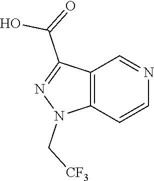 [SMILES: O=C(O)c1nn(CC(F)(F)F)c2ccncc12] | |
| 8g | 1-(2,2,2- trifluoroethyl)-1H- pyrazolo[4,3- b]pyridine-3- carboxylic acid | 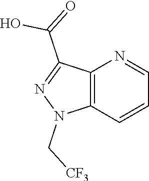 [SMILES: O=C(O)c1nn(CC(F)(F)F)c2cccnc12] | UPLC-MS: Rₜ = 0.65 min. MS (ESI+): m/z = 246.4 [M + H]⁺. |
| 8h | 1-(2,2,2- trifluoroethyl)-1H- pyrazolo[3,4- c]pyridine-3- carboxylic acid | 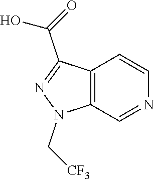 [SMILES: O=C(O)c1nn(CC(F)(F)F)c2cnccc12] | UPLC-MS: Rₜ = 0.58 min. MS (ESI+): m/z = 246.4 [M + H]⁺. |
| 8i | 1-(2-hydroxyethyl)- 1H-indazole-3- carboxylic acid | 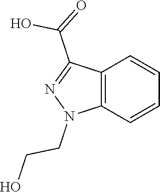 [SMILES: O=C(O)c1nn(CCO)c2ccccc12] | UPLC-MS: Rₜ = 0.65 min. MS (ESI+): m/z = 207.3 [M + H]⁺. |
| 8j | 1-(2,2,2- trifluoroethyl)-1H- pyrazolo[3,4- b]pyridine-3- carboxylic acid | 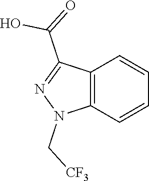 [SMILES: O=C(O)c1nn(CC(F)(F)F)c2ccccc12] | UPLC-MS: Rₜ = 0.80 min. MS (ESI+): m/z = 246.4 [M + H]⁺. |
| 8k | 1-(2-methoxyethyl)- 1H-indazole-3- carboxylic acid | 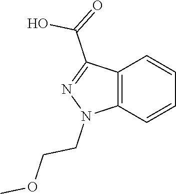 [SMILES: COCCn1nc(C(=O)O)c2ccccc21] | UPLC-MS: Rₜ = 0.82 min. MS (ESI+): m/z = 221.4 [M + H]⁺. |
| 8l | 4-phenyl-1-(2,2,2- trifluoroethyl)-1H- pyrazole-3- carboxylic acid | 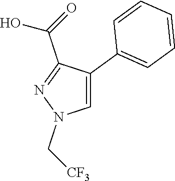 [SMILES: O=C(O)c1nn(CC(F)(F)F)cc1-c1ccccc1] | UPLC-MS: Rₜ = 0.47 min. MS (ESI+): m/z = 271.4 [M + H]⁺. |
| 8m | 5-phenyl-1-(2,2,2- trifluoroethyl)-1H- pyrazole-3- carboxylic acid | 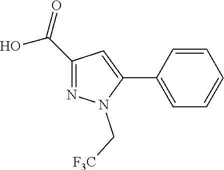 [SMILES: O=C(O)c1cc(-c2ccccc2)n(CC(F)(F)F)n1] | UPLC-MS: Rₜ = 1.00 min. MS (ESI+): m/z = 271.4 [M + H]⁺. |
| 8n | 1-((tetrahydrofuran- 3-yl)methyl)-1H- indazole-3- carboxylic acid | 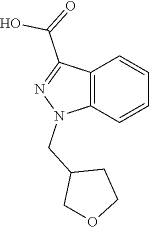 [SMILES: O=C(O)c1nn(CC2CCOC2)c2ccccc12] | UPLC-MS: Rₜ = 0.81 min. MS (ESI+): m/z = 247.4 [M + H]⁺. |
| 8o | 1-(2-hydroxy-2- methylpropyl)-1H- indazole-3- carboxylic acid | 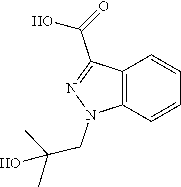 [SMILES: CC(C)(O)Cn1nc(C(=O)O)c2ccccc21] | UPLC-MS: Rₜ = 0.77 min. MS (ESI+): m/z = 235.4 [M + H]⁺. |
| 8p | 1-((tetrahydrofuran- 2-yl)methyl)-1H- indazole-3- carboxylic acid | 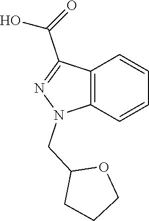 [SMILES: O=C(O)c1nn(CC2CCCO2)c2ccccc12] | UPLC-MS: Rₜ = 0.87 min. MS (ESI+): m/z = 247.4 [M + H]⁺. |
| 8q | 1-(prop-2-yn-1-yl)- 1H-indazole-3- carboxylic acid | 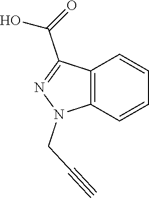 | ¹H NMR (400 MHz, DMSO-d6) δ: 13.17 (br s, 1 H), 8.08-8.13 (m, 1 H), 7.82 − 7.87 (m, 1 H), 7.53 (ddd, J = 8.43, 7.03, 1.01 Hz, 1 H), 7.36 (ddd, J = 8.05, 7.03, 0.89 Hz, 1 H), 5.48 (d, J = 2.53 Hz, 2 H), 3.50 (t, J = 3.15 Hz, 1 H). UPLC-MS: Rₜ = 0.82 min. MS (ESI+): m/z = 201.2 [M + H]⁺. prep-HPLC (Method described herein, Water (Acid): MeCN, gradient C) |
| 8r | 1-(but-2-yn-1-yl)- 1H-indazole-3- carboxylic acid | 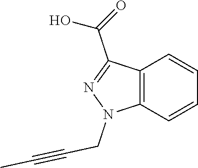 [SMILES: CC#CCn1nc(C(=O)O)c2ccccc21] | ¹H NMR (500 MHz, DMSO-d6) δ: 12.92-13.35 (m, 1H), 8.10 (m, 1H), 7.82 (d, 1H), 7.51 (m, 1H), 7.34 (m, 1H), 5.39 (m, 2H), 1.79 (m, 3H). LC-MS: Rₜ = 0.96 min. MS (ESI+): m/z = 215.1 [M + H]⁺. prep-HPLC (Method described herein, Water (Acid): MeCN, gradient C) |
| 8s | 1-((5- methylisoxazol-3- yl)methyl)-1H- indazole-3- carboxylic acid | 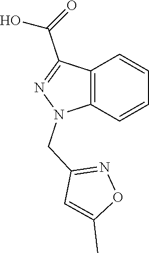 [SMILES: Cc1cc(Cn2nc(C(=O)O)c3ccccc32)no1] | UPLC-MS: Rₜ = 0.88 min. MS (ESI+): m/z = 258.4 [M + H]⁺. |
| 8t | 1-((5-methyl-1,2,4- oxadiazol-3- yl)methyl)-1H- indazole-3- carboxylic acid | 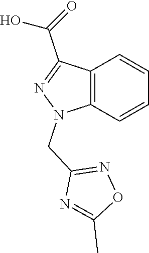 [SMILES: Cc1nc(Cn2nc(C(=O)O)c3ccccc32)no1] | UPLC-MS: Rₜ = 0.80 min. MS (ESI+): m/z = 259.4 [M + H]⁺. |
| 8u | 1-(2,2,2- trifluoroethyl)-1H- indole-3-carboxylic acid | 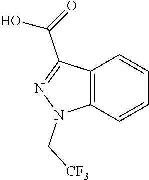 [SMILES: O=C(O)c1nn(CC(F)(F)F)c2ccccc12] | UPLC-MS: Rₜ = 0.98 min. MS (ESI+): m/z = 244.3 [M + H]⁺. |
| 8v | 2-methyl-1-(2,2,2- trifluoroethyl)-1H- indole-3-carboxylic acid | 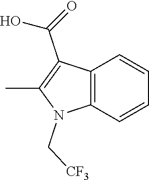 [SMILES: Cc1c(C(=O)O)c2ccccc2n1CC(F)(F)F] | UPLC-MS: Rₜ = 1.03 min. MS (ESI+): m/z = 258.4 [M + H]⁺. |
| 8w | 5-methyl-1-(2,2,2- trifluoroethyl)-1H- indazole-3- carboxylic acid | 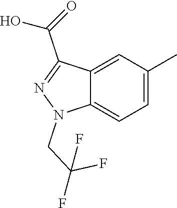 [SMILES: Cc1ccc2c(c1)c(C(=O)O)nn2CC(F)(F)F] | ¹H NMR (500 MHz, DMSO-d6) δ: 13.22 (br s, 1H), 7.91 (s, 1H), 7.79 (d, 1H), 7.39 (m, 1H), 5.57 (m, 2H), 2.46 (s, 3H). UPLC-MS: Rₜ = 1.00 min. MS (ESI+): m/z = 259.2 [M + H]⁺. prep-HPLC (Method described herein, Water (Acid): MeCN, gradient C) |
| 8x | 5-fluoro-1-(2,2,2- trifluoroethyl)-1H- indazole-3- carboxylic acid | 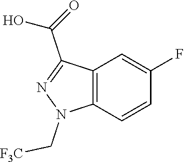 [SMILES: O=C(O)c1nn(CC(F)(F)F)c2ccc(F)cc12] | UPLC-MS (method 4): Rₜ = 0.95 min. MS (ESI+): m/z = 263.2 [M + H]⁺. |
| 8y | 1-((1,4-dioxan-2- yl)methyl)-1H- indazole-3- carboxylic acid | 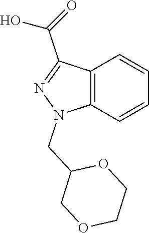 [SMILES: O=C(O)c1nn(CC2COCCO2)c2ccccc12] | UPLC-MS: Rₜ = 0.80 min. MS (ESI+): miz = 263.4 [M + H]⁺. |
| 8z | 5-methoxy-1-(2,2,2- trifluoroethyl)-1H- indazole-3- carboxylic acid | 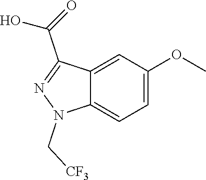 [SMILES: COc1ccc2c(c1)c(C(=O)O)nn2CC(F)(F)F] | ¹H NMR (600 MHz, DMSO-d6) δ: 13.02-13.43 (m, 1H), 7.83 (d, 1H), 7.46 (d, 1H), 7.21 (m, 1H), 5.58 (m, 2H), 3.84 (s, 3H). UPLC (method 6): Rₜ = 0.93 min. MS (ESI+): m/z = 275.5 [M + H]⁺. prep-HPLC (Method described herein, Water (Acid): MeCN, gradient C) |
| 8aa | 1-((tetrahydro-2H- pyran-3-yl)methyl)- 1H-indazole-3- carboxylic acid | 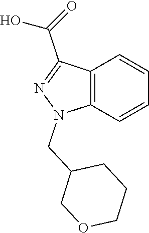 [SMILES: O=C(O)c1nn(CC2CCCOC2)c2ccccc12] | UPLC-MS: Rₜ = 0.90 min. MS (ESI+): m/z = 261.4 [M + H]⁺. |
| 8ab | 1-(1- cyclopropylethyl)- 1H-indazole-3- carboxylic acid | 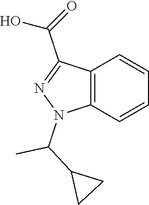 [SMILES: CC(C1CC1)n1nc(C(=O)O)c2ccccc21] | ¹H NMR (500 MHz, DMSO-d6) δ: 13.00 (s, 1H), 8.04-8.13 (m, 1H), 7.83 (d, 1H), 7.44 (m, 1H), 7.27-7.34 (m, 1H), 4.31 (m, 1H), 1.63 (d, 3H), 1.37- 1.48 (m, 1H), 0.63 (tm, 1H), 0.45 (m, 1H), 0.31-0.39 (m, 1H), 0.27 (m, 1H). UPLC-MS: Rₜ = 1.02 min. MS (ESI+): m/z =231.3 [M + H]⁺. |
| 8ac | 1-(1,1,1- trifluoropropan-2- yl)-1H-indazole-3- carboxylic acid | 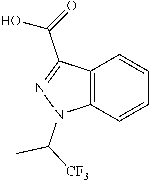 [SMILES: CC(n1nc(C(=O)O)c2ccccc21)C(F)(F)F] | UPLC-MS (method 4): Rₜ = 0.99 min. MS (ESI+): m/z = 259.2 [M + H]⁺. |
| 8ad | 4-methyl-1-(2,2,2- trifluoroethyl)-1H- indazole-3- carboxylic acid | 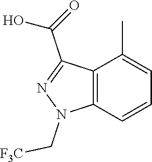 [SMILES: Cc1cccc2c1c(C(=O)O)nn2CC(F)(F)F] | UPLC-MS: Rₜ = 1.00 min. MS (ESI+): m/z = 259.2 [M + H]⁺. |
| 8ae | (S)-1- ((tetrahydrofuran-2- yl)methyl)-1H- indazole-3- carboxylic acid | 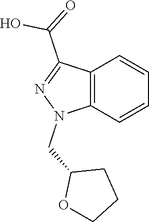 [SMILES: O=C(O)c1nn(C[C@@H]2CCCO2)c2ccccc12] | UPLC-MS: Rₜ = 0.87 min. MS (ESI+): m/z = 247.2 [M + H]⁺. |
| 8af | (R)-1- ((tetrahydrofuran-2- yl)methyl)-1H- indazole-3- carboxylic acid | 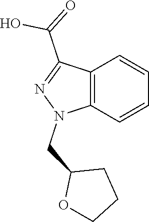 [SMILES: O=C(O)c1nn(C[C@H]2CCCO2)c2ccccc12] | ¹H NMR (400 MHz, DMSO-d6) δ: 13.02 (br s, 1 H), 8.03 − 8.13 (m, 1 H), 7.75 − 7.86 (m, 1 H), 7.41 − 7.51 (m, 1 H), 7.25 − 7.36 (m, 1 H), 4.52 − 4.58 (m, 2 H), 4.24 − 4.34 (m, 1 H), 3.65 − 3.74 (m, 1 H), 3.52 − 3.63 (m, 1 H), 1.93 − 2.04 (m, 1 H), 1.72 − 1.83 (m, 2 H), 1.60 − 1.72 (m, 1 H). UPLC-MS (method 6): Rₜ = 0.87 min. MS (ESI+): m/z = 247.5 [M + H]⁺. |
| 8ag | 6-methyl-1-(2,2,2- trifluoroethyl)-1H- indazole-3- carboxylic acid | 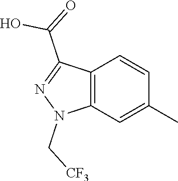 [SMILES: Cc1ccc2c(C(=O)O)nn(CC(F)(F)F)c2c1] | UPLC-MS (method 4): Rₜ = 1.01 min. MS (ESI+): m/z = 259.2 [M + H]⁺. |
| 8ah | 1-(2,2,2- trifluoroethyl)- 1,4,5,6- tetrahydrocyclopenta [c]pyrazole-3- carboxylic acid | 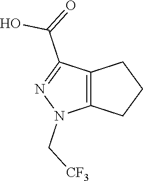 [SMILES: O=C(O)c1nn(CC(F)(F)F)c2c1CCC2] | UPLC-MS (method 4): Rₜ = 0.86 min. MS (ESI+): m/z = 235.2 [M + H]⁺. |
| 8ai | 1-(1-(tetrahydro-2H- pyran-4-yl)ethyl)- 1H-indazole-3- carboxylic acid | 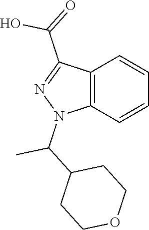 [SMILES: CC(C1CCOCC1)n1nc(C(=O)O)c2ccccc21] | UPLC-MS: Rₜ = 0.92 min. MS (ESI+): m/z = 275.3 [M + H]⁺. |
| 8aj | 3-phenyl-1-(2,2,2- trifluoroethyl)-1H- pyrazole-5- carboxylic acid | 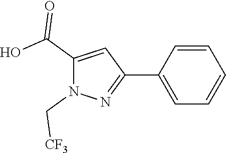 [SMILES: O=C(O)c1cc(-c2ccccc2)nn1CC(F)(F)F] | UPLC-MS: Rₜ = 1.12 min. MS (ESI+): m/z = 271.4 [M + H]⁺. |
| 8ak | 2-(2,2,2- trifluoroethyl)-2H- indazole-3- carboxylic acid | 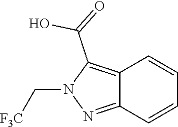 [SMILES: O=C(O)c1c2ccccc2nn1CC(F)(F)F] | UPLC-MS: Rₜ = 0.97 min. MS (ESI+): m/z = 245.3 [M + H]⁺. |
| 8al | 1- (cyclopropylmethyl)- 1H-indazole-3- carboxylic acid | 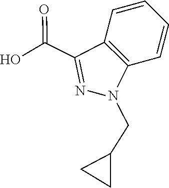 [SMILES: O=C(O)c1nn(CC2CC2)c2ccccc12] | UPLC-MS: Rₜ = 0.50 min. MS (ESI+): m/z = 217.4 [M + H]⁺. |
| 8am | 1-isobutyl-1H- indazole-3- carboxylic acid | 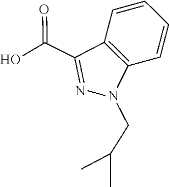 [SMILES: CC(C)Cn1nc(C(=O)O)c2ccccc21] | UPLC-MS: Rₜ = 0.54 min. MS (ESI+): m/z = 219.4 [M + H]⁺. |
| 8an | 1- (cyclobutylmethyl)- 1H-indazole-3- carboxylic acid | 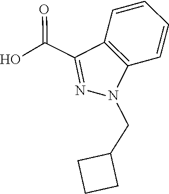 [SMILES: O=C(O)c1nn(CC2CCC2)c2ccccc12] | UPLC-MS: Rₜ = 0.56 min. MS (ESI+): m/z = 231.4 [M + H]⁺. |
| 8ao | 1-((tetrahydro-2H- pyran-4-yl)methyl)- 1H-indazole-3- carboxylic acid | 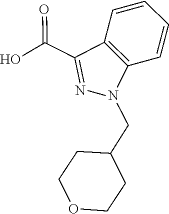 [SMILES: O=C(O)c1nn(CC2CCOCC2)c2ccccc12] | UPLC-MS: Rₜ = 0.86 min. MS (ESI+): m/z = 261.4 [M + H]⁺. |

### Intermediate 8ap: 4-cyclopropyl-1-(2,2,2-trifluoroethyl)-1H-pyrazole-3-carboxylic acid

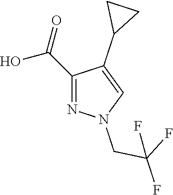 [SMILES: O=C(O)c1nn(CC(F)(F)F)cc1C1CC1]

A mixture of intermediate 7q (394 mg, 1 Eq, 1.37 mmol), cyclopropylboronic acid (129.7 mg, 1.1 Eq, 1.51 mmol), potassium phosphate (1.11 g, 3.8 Eq, 5.22 mmol), PdOAc₂ (30.8 mg, 0.1 Eq, 137 μmol), tricyclohexylphosphine (38.5 mg, 0.1 Eq, 137 μmol), toluene (7 mL) and water (4 mL) was stirred at 120° C. overnight. The mixture was evaporated to dryness. DCM and H₂O were added, and the aqueous layer was extracted with DCM (3×). The combined organics were evaporated to dryness. Crude was purified via prep-HPLC (Method described herein, Water (Acidic): MeCN, gradient C) to give the title compound (23 mg, 7.2%).

¹H-NMR (400 MHz, DMSO-d₆) δ: 12.79 (br s, 1H), 7.56 (s, 1H), 5.00-5.19 (m, 2H), 2.16-2.26 (m, 1H), 0.81-0.95 (m, 2H), 0.42-0.52 (m, 2H).

### Intermediate 8ba: 1-((1-cyanocyclopropyl)methyl)-1H-indazole-3-carboxylic acid

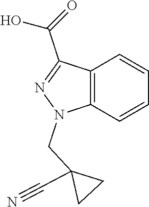 [SMILES: N#CC1(Cn2nc(C(=O)O)c3ccccc32)CC1]

Commercially available 1H-indazole-3-carboxylic acid (350 mg, 1.0 Eq, 2.16 mmol) was dissolved in DMF (5 mL), cooled to 0° C., then sodium hydride (216 mg, 60% Wt, 2.5 Eq, 5.38 mmol) was added, stirred 5 min at 0° C. and 25 min at 25° C. Cooled again in an ice-bath and 1-(bromomethyl)cyclopropane-1-carbonitrile (414 mg, 1.2 Eq, 2.59 mmol) was added. Stirred at rt overnight. EtOAc and sat. NaHCO₃were added, stirred couple of minutes, then organic layer was separated and washed with water and brine. Filtered and evaporated. The remaining crude was purified via prep-HPLC (Method described herein, Water (Acidic): MeCN, gradient C) to give the title product (140 mg, 26.9%).

¹H-NMR (600 MHz, DMSO-d₆) δ: 13.18 (br s, 1H), 8.08-8.15 (m, 1H), 7.94 (d, 1H), 7.52 (ddd, 1H), 7.31-7.39 (m, 1H), 4.77 (s, 2H), 1.37 (d, 4H).

UPLC-MS: Rₜ=0.81 min. MS (ESI+): m/z=242.4 [M+H]⁺.

Intermediates 8bb and 8bc were prepared in analogy to intermediate 8ba using bromide/chloride/triflate as an alkylating reagent.

| Int. | Name | Structure | Analytical Data |
|---|---|---|---|
| 8bb | 1-(cyanomethyl)-1H- indazole-3-carboxylic acid | 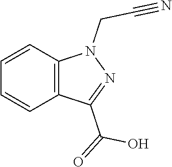 | ¹H NMR (500 MHz, DMSO-d₆) δ: 13.15-13.58 (m, 1H), 8.11- 8.18 (m, 1H), 7.91 (d, 1H), 7.60 (m, 1H), 7.41 (m, 1H), 5.97 (s, 2H). UPLC-MS: Rₜ = 0.73 min. MS (ESI+): m/z = 202.3 [M + H]⁺. |
| 8bc | 7-methyl-1-(2,2,2- trifluoroethyl)-1H- indazole-3-carboxylic acid | 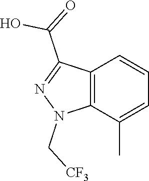 [SMILES: Cc1cccc2c(C(=O)O)nn(CC(F)(F)F)c12] | ¹H NMR (600 MHz, DMSO-d6) δ: 13.20-13.49 (m, 1H), 8.02 (d, 1H), 7.30-7.34 (m, 1H), 7.26 (m, 1H), 5.62 (m, 2H), 2.72 (s, 3H). UPLC-MS (method 6): Rₜ = 0.99 min. MS (ESI+): m/z = 259.5 [M + H]⁺. prep-HPLC (Method described herein, Water (Acidic): MeCN, gradient C) |

### Intermediate 8bd: 1-(2,2,2-trifluoroethyl)-1H-indazole-3-carboxylic acid

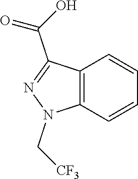 [SMILES: O=C(O)c1nn(CC(F)(F)F)c2ccccc12]

Methyl 1H-indazole-3-carboxylate (6.3 g, 1 Eq, 36 mmol) was dissolved in THF (130 mL) under an inert atmosphere and the solution was cooled with an ice bath, at this point sodium hydride (1.6 g, 60% Wt, 1.1 Eq, 39 mmol) was added portion wise over few minutes. The reaction was stirred at 0° C. for 5 min and at rt for an additional 15 minutes. The solution was cooled again with an ice bath and 2,2,2-trifluoroethyl trifluoromethanesulfonate (10 g, 1.2 Eq, 43 mmol) as a solution in THF (30 mL) was added dropwise over a few minutes. The reaction was stirred overnight at rt, TLC check (Hex EA 7:3) in the morning showed complete consumption of SM and formation of a single less polar spot. The reaction was diluted with water (80 mL), cooled with an ice bath and a solution of LiOH (5.1 g, 6 Eq, 0.21 mol) in water (80 mL) was added over few minutes, the reaction was stirred four hours. TLC showed completed consumption of N-alkylated methyl ester, the mixture was cooled with an ice bath and the pH was adjusted to acidic with HCl 1N, the mixture was further diluted with water (500 mL), the mixture was left stirring for 20 minutes in the ice bath and then the solid was collected by vacuum filtration, washed with hexane (×3) and dried over night at high vacuum.

¹H NMR (DMSO-d₆, 400 MHz) S 13.34 (br s, 1H), 8.1-8.1 (m, 1H), 7.92 (d, 1H, J=8.4 Hz), 7.57 (ddd, 1H, J=1.3, 7.1, 8.4 Hz), 7.38 (ddd, 1H, J=0.8, 7.2, 8.0 Hz), 5.63 (q, 2H, J=9.1 Hz).

### Intermediate 9a: 1-(2,2,2-trifluoroethyl)-1H-indazole-3-carbonyl chloride

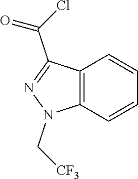 [SMILES: O=C(Cl)c1nn(CC(F)(F)F)c2ccccc12]

1-(2,2,2-Trifluoroethyl)-1H-indazole-3-carboxylic acid (2015 mg, 1 Eq, 8.252 mmol) was suspended in DCM (50 mL) under an inert atmosphere and cooled with an ice bath. At this point, oxalyl chloride (1.257 g, 866.8 μL, 1.2 Eq, 9.903 mmol) was added followed by a drop of DMF, the reaction was stirred at 0° C. for 10 min and then at rt for one hour, over the time the suspension became a solution. The solvent was removed in vacuo, the residue taken up again with DCM and evaporated twice to give a powdery residue that was directly used in the next step.

### Intermediate 10a: tert-Butyl 2-(2-(3-(1-(2,2-difluoroethyl)-1H-indazole-3-carboxamido)-4-(piperidin-1-yl)benzamido)-5-fluorophenyl)acetate

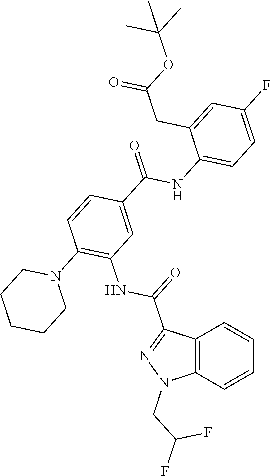 [SMILES: CC(C)(C)OC(=O)Cc1cc(F)ccc1NC(=O)c1ccc(N2CCCCC2)c(NC(=O)c2nn(CC(F)F)c3ccccc23)c1]

Amide coupling 1: A mixture of intermediate 6 (99.0 mg, 1.05 Eq, 232 μmol), 1-(2,2-difluoroethyl)-1Hindazole-3-carboxylic acid (49.9 mg, 1 Eq, 221 μmol) and HATU (168 mg, 2 Eq, 441 μmol) were dissolved in DMSO (5 mL) and then DIPEA (143 mg, 192 μL, 5 Eq, 1.10 mmol) was added. The mixture was stirred overnight at 25° C. Additional 2 eq of DIPEA and 1 eq of HATU were added and the mixture stirred until complete conversion. DCM and water were added. The organic phase was extracted. The aqueous phase was washed with DCM. The organic phases were combined, dried on sodium sulfate, filtered and the solvent was removed under reduced pressure. Crude was purified by prep-HPLC (Method described herein, Water (Basic): MeCN, gradient F): to give 76.4 mg (120 μmol) of the title compound.

UPLC-MS: Rₜ=1.57 min. MS (ESI+): m/z=636.8 [M+H]⁺.

Intermediate 10b: tert-butyl 2-(5-fluoro-2-(3-(1-methylimidazo[1,5-a]pyridine-3-carboxamido)-4-(piperidin-1-yl)benzamido)phenyl)acetate

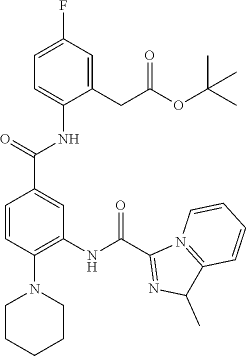

Amide coupling 2: To a mixture of 1-methylimidazo[1,5-a]pyridine-3-carboxylic acid (100 mg, 1 Eq, 568 μmol) in DCM (5 mL) was added oxalyl dichloride (256 mg, 5 Eq, 2.84 mmol) and catalytic amount of DMF (2.07 mg, 2.20 μL, 0.05 Eq, 28.4 μmol) at 0° C. The resulting mixture was stirred at 25° C. for 2 hours. The mixture was evaporated to dryness. The obtained 1-methylimidazo[1,5-a]pyridine-3-carbonyl chloride (110 mg, 1.1 Eq, 565 μmol) was dissolved in DCM (8 mL) and cooled to 0° C. Triethylamine (52.0 mg, 1 Eq, 514 μmol) was added, followed by intermediate 6 (220 mg, 1 Eq, 514 μmol). The mixture was stirred at 25° C. overnight. The mixture was evaporated to dryness and purified by Biotage flash silica gel chromatography (SNAP KP-Sil 25g, Hex/EtOAc=10/0 to 7/3).

UPLC-MS: Rₜ=1.73 min. MS (EST+): m/z=586.8 [M+H]⁺.

### Intermediate 10c: tert-butyl 2-(2-(3-(1-(cyanomethyl)-1H-indazole-3-carboxamido)-4-(piperidin-1-yl)benzamido)-5-fluorophenyl)acetate

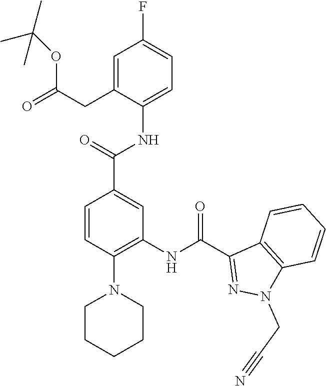 [SMILES: CC(C)(C)OC(=O)Cc1cc(F)ccc1NC(=O)c1ccc(N2CCCCC2)c(NC(=O)c2nn(CC#N)c3ccccc23)c1]

Amide coupling 3: tert-butyl 2-(2-(3-amino-4-(piperidin-1-yl)benzamido)-5-fluorophenyl)acetate (0.13 g, 1 Eq, 0.30 mmol) was dissolved in DCM/Py 1:1 (2 mL), to it a mixture of 1-(cyanomethyl)-1H-indazole-3-carbonyl chloride (99 mg, 1.5 Eq, 0.45 mmol) in DCM (2 mL) was added dropwise at rt, reaction stirred 2h at rt, UPLC shows main peak of desired mass. The reaction was diluted with DCM and HC11 N/ice, the organic phase was washed with a saturated solution of NaHCO₃, brine and dried over sodium sulfate. The residue was purified by automated flash column chromatography with a gradient 10-25-40% EtOAc in Hex to give 118 mg of the desired product (0.19 mmol, 64%).

UPLC-MS: Rₜ=1.53 min. MS (EST+): m/z=611.8 [M+H]⁺.

Intermediates 10d-10av were prepared in analogy to intermediate 10a and 10b.

Intermediate 10h was prepared using commercially available acid.

In method described herein, DMSO or DMF were used as solvents and DIPEA or Et₃N were used as a base.

| | | Structure/ | |
|---|---|---|---|
| Int. | Name | Coupling method | Analytical Data |
| 10d | tert-butyl 2-(5-fluoro- 2-(4-(piperidin-1-yl)- 3-(1-(3,3,3- trifluoropropyl)-1H- indazole-3- carboxamido) benzamido) phenyl)acetate | 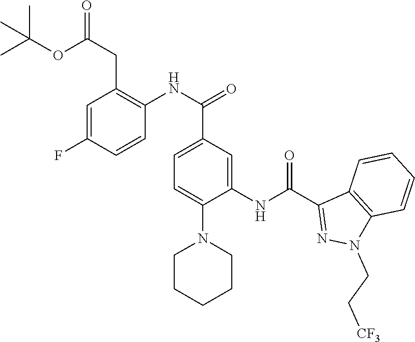 [SMILES: CC(C)(C)OC(=O)Cc1cc(F)ccc1NC(=O)c1ccc(N2CCCCC2)c(NC(=O)c2nn(CCC(F)(F)F)c3ccccc23)c1] | 1H NMR (400 MHz, DMSO-d6) δ: 10.08 (s, 1H), 9.93 (s, 1H), 8.97-9.03 (m, 1H), 8.25-8.32 (m, 1H), 7.93 (d, J = 8.62 Hz, 1H), 7.72 (dd, J = 8.36, 2.03 Hz, 1H), 7.53-7.59 (m, 1H), 7.36-7.44 (m, 3H), 7.14-7.22 (m, 2H), 4.82- 4.90 (m, 2H), 3.67 (s, 2H), 3.03- 3.18 (m, 2H), 2.87-2.94 (m, 4H), 1.79-1.90 (m, 4H), 1.58-1.72 (m, 2H), 1.29 (m, 9H). UPLC-MS: Rₜ = 1.63 min. MS (ESI+): m/z = 668.8 [M + H]⁺. |
| 10e | tert-butyl 2-(2-(3-(1- (2-((tert- butoxycarbonyl) amino)ethyl)-1H- indazole-3- carboxamido)-4- (piperidin-1- yl)benzamido)-5- fluorophenyl)acetate | 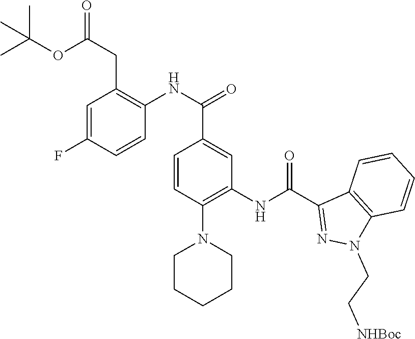 [SMILES: CC(C)(C)OC(=O)Cc1cc(F)ccc1NC(=O)c1ccc(N2CCCCC2)c(NC(=O)c2nn(CCNC(=O)OC(C)(C)C)c3ccccc23)c1] | ¹H NMR (400 MHz, DMSO-d6) δ: 10.03 (s, 1H), 9.88 (s, 1H), 9.01 (d, J = 2.03 Hz, 1H), 8.26 (d, J = 8.11 Hz, 1H), 7.67-7.78 (m, 2H), 7.48-7.54 (m, 1H), 7.33- 7.43 (m, 3H), 7.13-7.23 (m, 2H), 6.92-6.98 (m, 1H), 4.53-4.62 (m, 2H), 3.67 (s, 2H), 3.44-3.53 (m, 2H), 2.87-2.98 (m, 4H), 1.81- 1.91 (m, 4H), 1.60-1.71 (m, 2H), 1.31 (m, 9H), 1.23 (m, 9H). UPLC-MS (method 4): Rₜ = 1.65 min. MS (ESI+): m/z = 715.9 [M + H]⁺. |
| 10f | tert-butyl 4-((3-((5- ((2-(2-(tert-butoxy)- 2-oxoethyl)-4- fluorophenyl) carbamoyl)- 2-(piperidin-1- yl)phenyl)carbamoyl)- 1H-indazol-1- yl)methyl)piperidine- 1-carboxylate | 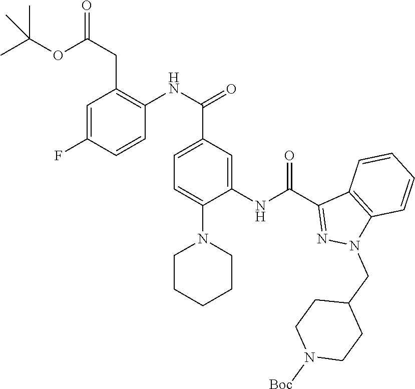 [SMILES: CC(C)(C)OC(=O)Cc1cc(F)ccc1NC(=O)c1ccc(N2CCCCC2)c(NC(=O)c2nn(CC3CCN(C(=O)OC(C)(C)C)CC3)c3ccccc23)c1] | UPLC-MS: Rₜ = 1.71 min. MS (ESI+): m/z = 770.1 [M + H]⁺. |
| | | Method described | |
| | | herein | |
| 10g | tert-butyl 2-(5-fluoro- 2-(4-(piperidin-1-yl)- 3-(1-(2,2,2- trifluoroethyl)-1H- pyrazolo[4,3- c]pyridine-3- carboxamido) benzamido) phenyl)acetate | 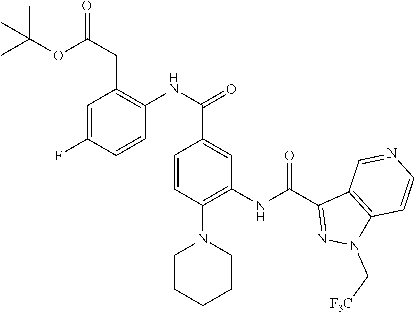 [SMILES: CC(C)(C)OC(=O)Cc1cc(F)ccc1NC(=O)c1ccc(N2CCCCC2)c(NC(=O)c2nn(CC(F)(F)F)c3ccncc23)c1] | ¹H NMR (400 MHz, DMSO-d6) δ: 10.03 (s, 1H), 9.94 (s, 1H), 9.53-9.60 (m, 1H), 8.98-9.00 (m, 1H), 8.63 (d, J = 5.83 Hz, 1H), 8.01-8.05 (m, 1H), 7.76 (dd, J = 8.36, 2.03 Hz, 1H), 7.37-7.45 (m, 2H), 7.13-7.23 (m, 2H), 5.66-5.75 (m, 2H), 3.67 (s, 2H), 2.86-2.98 (m, 4H), 1.76-1.87 (m, 4H), 1.54-1.66 (m, 2H), 1.29 (s, 9H). UPLC-MS: Rₜ = 1.51 min. MS (ESI+): m/z = 656.0 [M + H]⁺. |
| 10h | tert-butyl 2-(5-fluoro- 2-(3-(imidazo[1,2- a]pyridine-2- carboxamido)-4- (piperidin-1- yl)benzamido)phenyl) acetate | 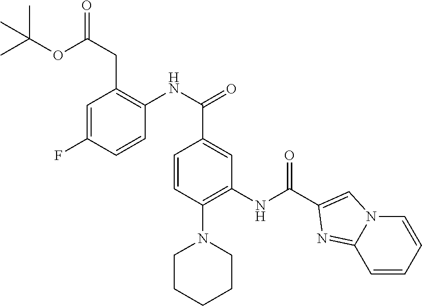 [SMILES: CC(C)(C)OC(=O)Cc1cc(F)ccc1NC(=O)c1ccc(N2CCCCC2)c(NC(=O)c2cn3ccccc3n2)c1] | ¹H NMR (400 MHz, DMSO-d6) δ: 10.26 (s, 1H), 9.87 (s, 1H), 8.96-8.99 (m, 1H), 8.60-8.64 (m, 1H), 8.57-8.59 (m, 1H), 7.69-7.73 (m, 1H), 7.64-7.68 (m, 1H), 7.38-7.43 (m, 2H), 7.31- 7.35 (m, 1H), 7.12-7.21 (m, 2H), 7.04 (td, J = 6.78, 1.14 Hz, 1H), 3.66 (s, 2H), 2.86-2.95 (m, 4H), 1.81-1.91 (m, 4H), 1.58-1.70 (m, 2H), 1.28 (s, 9H) UPLC-MS: Rₜ = 1.51 min. MS (ESI+): m/z = 572.8 [M + H]⁺. |
| 10i | tert-Butyl 2-(2-(3-(1- cyclopropyl-1H- indazole-3- carboxamido)-4- (piperidin-1- yl)benzamido)-5- fluorophenyl)acetate | 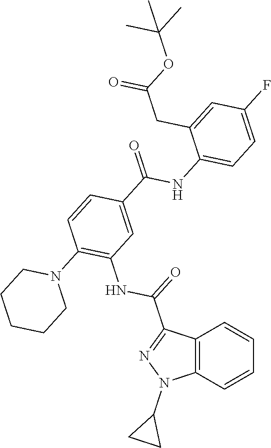 [SMILES: CC(C)(C)OC(=O)Cc1cc(F)ccc1NC(=O)c1ccc(N2CCCCC2)c(NC(=O)c2nn(C3CC3)c3ccccc23)c1] | UPLC-MS: Rₜ = 1.73 min. MS (ESI+): m/z = 612.9 [M + H]⁺. |
| 10j | tert-butyl 2-(5-fluoro- 2-(4-(piperidin-1-yl)- 3-(1-(2,2,2- trifluoroethyl)-1H- pyrazolo[4,3- b]pyridine-3- carboxamido) benzamido) phenyl)acetate | 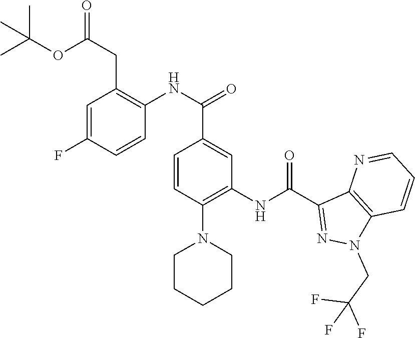 [SMILES: CC(C)(C)OC(=O)Cc1cc(F)ccc1NC(=O)c1ccc(N2CCCCC2)c(NC(=O)c2nn(CC(F)(F)F)c3cccnc23)c1] | ¹H NMR (400 MHz, DMSO-d6) δ: 11.27 (s, 1 H), 9.90 (s, 1 H), 9.02-9.11 (m, 1 H), 8.82-8.93 (m, 1 H), 8.45-8.64 (m, 1 H), 7.71-7.81 (m, 2 H), 7.36-7.47 (m, 2 H), 7.08-7.27 (m, 2 H), 5.69-5.86 (m, 2 H), 3.67 (s, 2 H), 2.85-2.93 (m, 4 H), 1.73- 1.87 (m, 4 H), 1.53-1.69 (m, 2 H), 1.22-1.32 (m, 9 H). UPLC-MS: Rₜ = 1.49 min. MS (ESI+): m/z = 655.8 [M + H]⁺. |
| 10k | tert-butyl 2-(5-fluoro- 2-(3-(1-(2- hydroxyethyl)-1H- indazole-3- carboxamido)-4- (piperidin-1- yl)benzamido)phenyl) acetate | 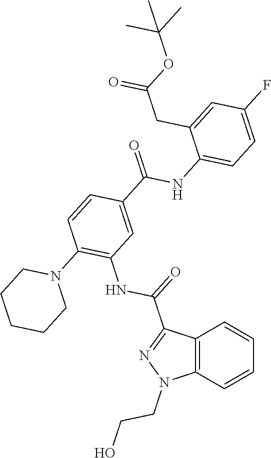 [SMILES: CC(C)(C)OC(=O)Cc1cc(F)ccc1NC(=O)c1ccc(N2CCCCC2)c(NC(=O)c2nn(CCO)c3ccccc23)c1] | UPLC-MS: Rₜ = 1.50 min. MS (ESI+): m/z = 616.8 [M + H]⁺. |
| 10l | tert-butyl 2-(5-fluoro- 2-(4-(piperidin-1-yl)- 3-(1-(2,2,2- trifluoroethyl)-1H- pyrazolo[3,4- c]pyridine-3- carboxamido) benzamido) phenyl)acetate | 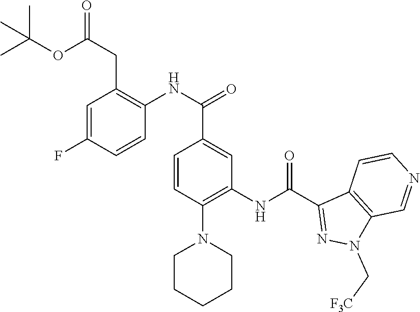 [SMILES: CC(C)(C)OC(=O)Cc1cc(F)ccc1NC(=O)c1ccc(N2CCCCC2)c(NC(=O)c2nn(CC(F)(F)F)c3cnccc23)c1] | ¹H NMR (400 MHz, DMSO-d6) δ: 10.02 (s, 1 H), 9.93 (s, 1 H), 9.50 (s, 1 H), 8.95-9.02 (m, 1 H), 8.50-8.59 (m, 1 H), 8.14- 8.25 (m, 1 H), 7.72-7.79 (m, 1 H), 7.36-7.44 (m, 2 H), 7.12- 7.25 (m, 2 H), 5.78-5.89 (m, 2 H), 3.67 (s, 2 H), 2.84-2.94 (m, 4 H), 1.76-1.87 (m, 4 H), 1.56- 1.70 (m, 2 H), 1.29 (s, 9 H). UPLC-MS: Rₜ = 1.52 min. MS (ESI+): m/z = 655.9 [M + H]⁺. |
| 10m | tert-butyl 2-(5-fluoro- 2-(4-(piperidin-1-yl)- 3-(1-(2,2,2- trifluoroethyl)-1H- pyrazolo[3,4- b]pyridine-3- carboxamido) benzamido) phenyl)acetate | 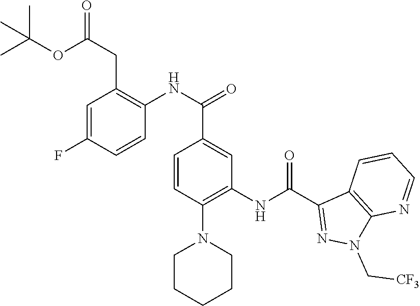 [SMILES: CC(C)(C)OC(=O)Cc1cc(F)ccc1NC(=O)c1ccc(N2CCCCC2)c(NC(=O)c2nn(CC(F)(F)F)c3ncccc23)c1] | ¹H NMR (400 MHz, DMSO-d6) δ: 10.04 (s, 1 H), 9.94 (s, 1 H), 8.95-9.01 (m, 1 H), 8.77-8.82 (m, 1 H), 8.71 (dd, J = 8.11, 1.52 Hz, 1 H), 7.72-7.80 (m, 1 H), 7.57 (dd, J = 8.11, 4.56 Hz, 1 H), 7.36-7.42 (m, 2 H), 7.19 (td, J = 9.12, 3.04 Hz, 2 H), 5.54- 5.63 (m, 2 H), 3.67 (s, 2 H), 2.87- 2.95 (m, 4 H), 1.74-1.87 (m, 4 H), 1.57-1.69 (m, 2 H), 1.29 (s, 9 H). UPLC-MS: Rₜ = 1.61 min. MS (ESI+): m/z = 655.9 [M + H]⁺. |
| 10n | tert-butyl 2-(5-fluoro- 2-(3-(1-(2- methoxyethyl)-1H- indazole-3- carboxamido)-4- (piperidin-1- yl)benzamido)phenyl) acetate | 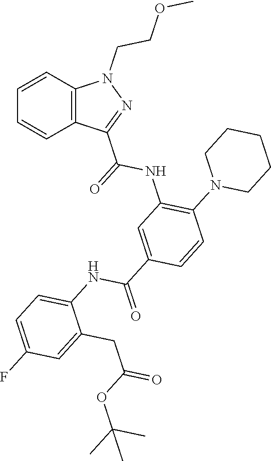 [SMILES: COCCn1nc(C(=O)Nc2cc(C(=O)Nc3ccc(F)cc3CC(=O)OC(C)(C)C)ccc2N2CCCCC2)c2ccccc21] | UPLC-MS: Rₜ = 1.60 min. MS (ESI+): m/z = 630.8 [M + H]⁺. Prep-HPLC: Method described herein, basic eluent, gradient F |
| 10o | tert-butyl 2-(5-fluoro- 2-(3-(4-phenyl-1- (2,2,2-trifluoroethyl)- 1H-pyrazole-3- carboxamido)-4- (piperidin-1- yl)benzamido)phenyl) acetate | 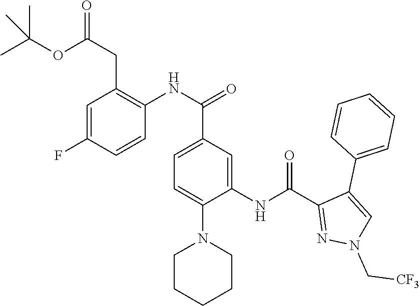 [SMILES: CC(C)(C)OC(=O)Cc1cc(F)ccc1NC(=O)c1ccc(N2CCCCC2)c(NC(=O)c2nn(CC(F)(F)F)cc2-c2ccccc2)c1] | ¹H NMR (400 MHz, DMSO-d6) δ: 9.94 (s, 1 H), 9.87 (s, 1 H), 8.87 (d, J = 2.03 Hz, 1 H), 8.26 (s, 1 H), 7.67-7.71 (m, 1 H), 7.62- 7.65 (m, 2 H), 7.30-7.43 (m, 5 H), 7.11-7.20 (m, 2 H), 5.26- 5.38 (m, 2 H), 3.63 (s, 2 H), 2.80- 2.93 (m, 4 H), 1.73-1.85 (m, 4 H), 1.54-1.66 (m, 2 H), 1.24- 1.31 (m, 9 H). UPLC-MS (method 3): Rₜ = 1.66 min. MS (ESI+): m/z = 680.8 [M + H]⁺. |
| 10p | tert-butyl 2-(5-fluoro- 2-(3-(5-phenyl-1- (2,2,2-trifluoroethyl)- 1H-pyrazole-3- carboxamido)-4- (piperidin-1- yl)benzamido)phenyl) acetate | 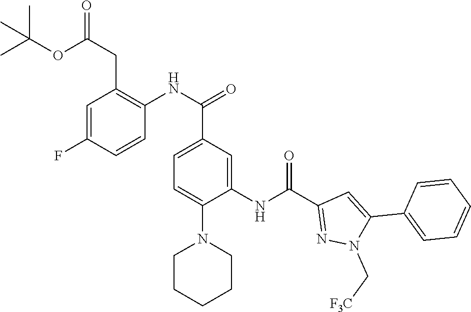 [SMILES: CC(C)(C)OC(=O)Cc1cc(F)ccc1NC(=O)c1ccc(N2CCCCC2)c(NC(=O)c2cc(-c3ccccc3)n(CC(F)(F)F)n2)c1] | ¹H NMR (400 MHz, DMSO-d6) δ: 9.85-9.95 (m, 2 H), 8.87- 9.00 (m, 1 H), 7.70-7.75 (m, 1 H), 7.52-7.59 (m, 5 H), 7.34- 7.42 (m, 2 H), 7.13-7.21 (m, 2 H), 7.03-7.05 (m, 1 H), 5.12- 5.22 (m, 2 H), 3.66 (s, 2 H), 2.85- 2.91 (m, 4 H), 1.76-1.85 (m, 4 H), 1.56-1.64 (m, 2 H), 1.25- 1.32 (m, 9 H). UPLC-MS: Rₜ = 1.65 min. MS (ESI+): m/z = 680.9 [M + H]⁺. |
| 10r | tert-butyl 2-(2-(3-(4- cyclopropyl-1-(2,2,2- trifluoroethyl)-1H- pyrazole-3- carboxamido)-4- (piperidin-1- yl)benzamido)-5- fluorophenyl)acetate | 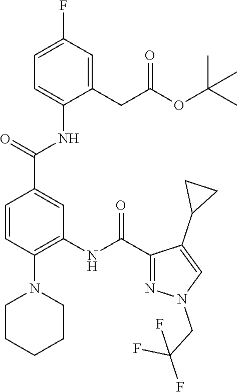 [SMILES: CC(C)(C)OC(=O)Cc1cc(F)ccc1NC(=O)c1ccc(N2CCCCC2)c(NC(=O)c2nn(CC(F)(F)F)cc2C2CC2)c1] | UPLC-MS: Rt = 1.63 min. MS (ESI+): m/z = 644.7 [M + H]⁺. |
| 10s | tert-butyl 2-(5-fluoro- 2-(3-(1-(2-hydroxy- 2-methylpropyl)-1H- indazole-3- carboxamido)-4- (piperidin-1- yl)benzamido)phenyl) acetate | 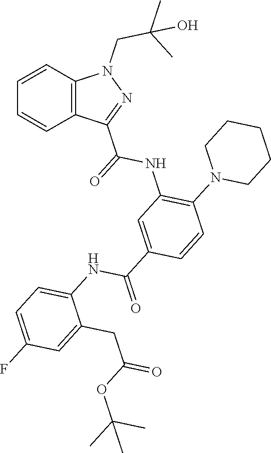 [SMILES: CC(C)(O)Cn1nc(C(=O)Nc2cc(C(=O)Nc3ccc(F)cc3CC(=O)OC(C)(C)C)ccc2N2CCCCC2)c2ccccc21] | UPLC-MS: Rₜ = 1.55 min. MS (ESI+): m/z = 644.9 [M + H]⁺. Prep-HPLC: Method described herein, acidic eluent, gradient F |
| 10t | tert-butyl 2-(5-fluoro- 2-(4-(piperidin-1-yl)- 3-(1- ((tetrahydrofuran-3- yl)methyl)-1H- indazole-3- carboxamido) benzamido) phenyl)acetate | 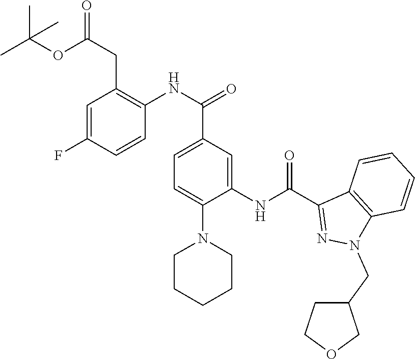 [SMILES: CC(C)(C)OC(=O)Cc1cc(F)ccc1NC(=O)c1ccc(N2CCCCC2)c(NC(=O)c2nn(CC3CCOC3)c3ccccc23)c1] | UPLC-MS: Rₜ = 1.58 min. MS (ESI+): m/z = 656.9 [M + H]⁺. |
| 10u | tert-butyl 2-(5-fluoro- 2-(4-(piperidin-1-yl)- 3-(1- ((tetrahydrofuran-2- yl)methyl)-1H- indazole-3- carboxamido) benzamido) phenyl)acetate | 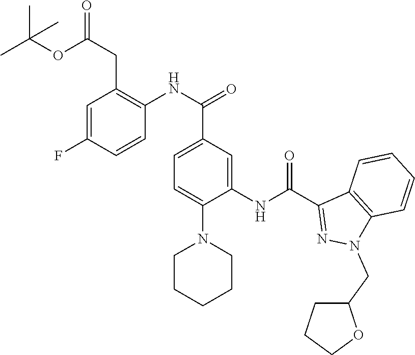 [SMILES: CC(C)(C)OC(=O)Cc1cc(F)ccc1NC(=O)c1ccc(N2CCCCC2)c(NC(=O)c2nn(CC3CCCO3)c3ccccc23)c1] | ¹H NMR (400 MHz, DMSO-d6) δ: 10.03 (s, 1 H), 9.92 (s, 1 H), 9.00 (d, J = 2.03 Hz, 1 H), 8.24- 8.28 (m, 1 H), 7.84-7.88 (m, 1 H), 7.69-7.73 (m, 1 H), 7.49- 7.55 (m, 1 H), 7.34-7.43 (m, 3 H), 7.13-7.22 (m, 2 H), 4.58- 4.64 (m, 2 H), 4.36-4.44 (m, 1 H), 3.58-3.76 (m, 4 H), 2.86- 2.97 (m, 4 H), 1.98-2.07 (m, 1 H), 1.74-1.89 (m, 7 H), 1.58- 1.69 (m, 2 H), 1.29 (s, 9 H). UPLC-MS: Rₜ = 1.63 min. MS (ESI+): m/z = 656.9 [M + H]⁺. |
| 10w | tert-butyl 2-(5-fluoro- 2-(4-(piperidin-1-yl)- 3-(1-(prop-2-yn-1- yl)-1H-indazole-3- carboxamido) benzamido) phenyl)acetate | 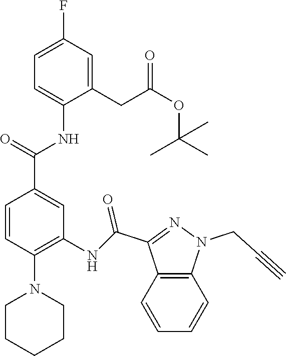 [SMILES: C#CCn1nc(C(=O)Nc2cc(C(=O)Nc3ccc(F)cc3CC(=O)OC(C)(C)C)ccc2N2CCCCC2)c2ccccc21] | UPLC-MS: Rt = 1.60 min. MS (ESI+): m/z = 610.9 [M + H]⁺. |
| 10x | tert-butyl 2-(2-(3-(1- (but-2-yn-1-yl)-1H- indazole-3- carboxamido)-4- (piperidin-1- yl)benzamido)-5- fluorophenyl)acetate | 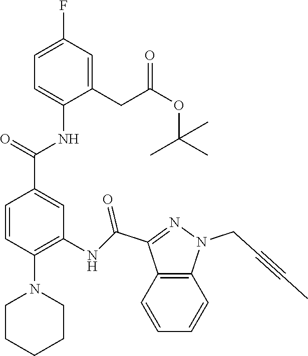 [SMILES: CC#CCn1nc(C(=O)Nc2cc(C(=O)Nc3ccc(F)cc3CC(=O)OC(C)(C)C)ccc2N2CCCCC2)c2ccccc21] | ¹H NMR (400 MHz, DMSO-d6) δ: 10.00 (s, 1 H), 9.91 (s, 1 H), 8.96-9.03 (m, 1 H), 8.26-8.30 (m, 1 H), 7.87-7.93 (m, 1 H), 7.68-7.75 (m, 1 H), 7.53-7.60 (m, 1 H), 7.34-7.44 (m, 3 H), 7.13-7.22 (m, 2 H), 5.45 (q, J = 2.03 Hz, 2 H), 3.68 (s, 2 H), 2.86-2.95 (m, 4 H), 1.83-1.90 (m, 4 H), 1.82 (t, J = 2.41 Hz, 3 H), 1.61-1.70 (m, 2 H), 1.29 (s, 9 H). UPLC-MS: Rₜ = 1.67 min. MS (ESI+): m/z = 624.8 [M + H]⁺. prep-HPLC (Method described herein, Water (Acidic): MeCN, gradient F) |
| 10y | tert-butyl 2-(5-fluoro- 2-(3-(1-((5- methylisoxazol-3- yl)methyl)-1H- indazole-3- carboxamido)-4- (piperidin-1- yl)benzamido)phenyl) acetate | 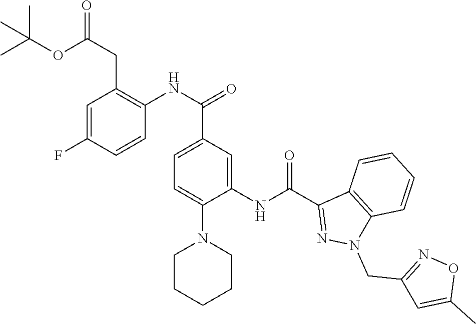 [SMILES: Cc1cc(Cn2nc(C(=O)Nc3cc(C(=O)Nc4ccc(F)cc4CC(=O)OC(C)(C)C)ccc3N3CCCCC3)c3ccccc32)no1] | ¹H NMR (400 MHz, DMSO-d6) δ: 9.97 (s, 1 H), 9.91 (s, 1 H), 8.95-9.00 (m, 1 H), 8.25-8.33 (m, 1 H), 7.92-7.99 (m, 1 H), 7.68-7.74 (m, 1 H), 7.55-7.60 (m, 1 H), 7.37-7.43 (m, 2 H), 7.34 (d, J = 8.36 Hz, 1 H), 7.13- 7.22 (m, 2 H), 6.10-6.12 (m, 1 H), 5.87 (s, 2 H), 3.67 (s, 2 H), 2.80-2.93 (m, 4 H), 2.32-2.37 (m, 3 H), 1.67-1.76 (m, 4 H), 1.54-1.65 (m, 2 H), 1.29 (m, 9 H). UPLC-MS: Rₜ = 1.57 min. MS (ESI+): m/z = 667.8 [M + H]⁺. |
| 10z | tert-butyl 2-(5-fluoro- 2-(3-(1-((5-methyl- 1,2,4-oxadiazol-3- yl)methyl)-1H- indazole-3- carboxamido)-4- (piperidin-1- yl)benzamido)phenyl) acetate | 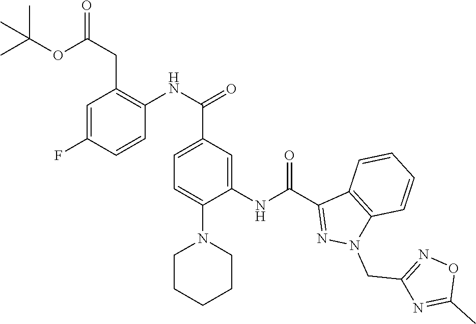 [SMILES: Cc1nc(Cn2nc(C(=O)Nc3cc(C(=O)Nc4ccc(F)cc4CC(=O)OC(C)(C)C)ccc3N3CCCCC3)c3ccccc32)no1] | ¹H NMR (400 MHz, DMSO-d6) δ: 9.95 (s, 1 H), 9.91 (s, 1 H), 8.94-8.98 (m, 1 H), 8.27-8.31 (m, 1 H), 7.95-7.99 (m, 1 H), 7.70 (dd, J = 8.36, 2.03 Hz, 1 H), 7.57-7.62 (m, 1 H), 7.38-7.44 (m, 2 H), 7.32-7.36 (m, 1 H), 7.13-7.22 (m, 2 H), 6.00 (s, 2 H), 3.67 (s, 2 H), 2.79-2.88 (m, 4 H), 2.55 (s, 3 H), 1.62-1.72 (m, 4 H), 1.53-1.62 (m, 2 H), 1.29 (s, 9 H). UPLC-MS: Rₜ = 1.53 min. MS (ESI+): m/z = 668.9 [M + H]⁺. |
| 10aa | tert-butyl 2-(2-(3-(1- ((1- cyanocyclopropyl) methyl)- 1H-indazole-3- carboxamido)-4- (piperidin-1- yl)benzamido)-5- fluorophenyl)acetate | 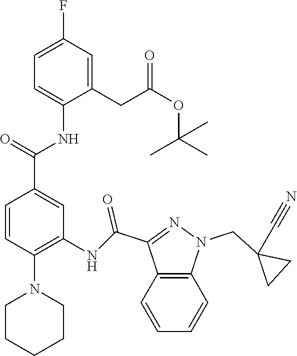 [SMILES: CC(C)(C)OC(=O)Cc1cc(F)ccc1NC(=O)c1ccc(N2CCCCC2)c(NC(=O)c2nn(CC3(C#N)CC3)c3ccccc23)c1] | UPLC-MS: Rₜ = 1.56 min. MS (ESI+): m/z = 652.0 [M + H]⁺. |
| 10ab | tert-butyl 2-(5-fluoro- 2-(4-(piperidin-1-yl)- 3-(1-(2,2,2- trifluoroethyl)-1H- indole-3- carboxamido) benzamido) phenyl)acetate | 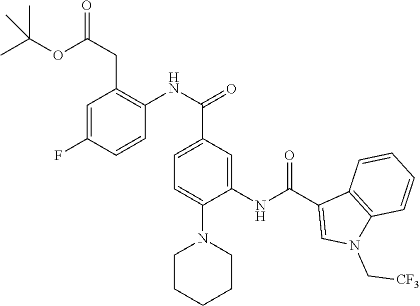 [SMILES: CC(C)(C)OC(=O)Cc1cc(F)ccc1NC(=O)c1ccc(N2CCCCC2)c(NC(=O)c2cn(CC(F)(F)F)c3ccccc23)c1] | ¹H NMR (400 MHz, DMSO-d6) δ: 9.87 (s, 1 H), 9.22 (s, 1 H), 8.70 (d, J = 2.03 Hz, 1 H), 8.30 (s, 1 H), 8.24-8.28 (m, 1 H), 7.75- 7.81 (m, 1 H), 7.68-7.75 (m, 1 H), 7.27-7.43 (m, 4 H), 7.11- 7.22 (m, 2 H), 5.40 (q, J = 9.29 Hz, 2 H), 3.66 (s, 2 H), 2.84- 2.97 (m, 4 H), 1.67-1.76 (m, 4 H), 1.53-1.61 (m, 2 H), 1.30 (m, 9 H). UPLC-MS: Rₜ = 1.58 min. MS (ESI+): m/z = 653.8 [M + H]⁺. |
| 10ac | tert-butyl 2-(5-fluoro- 2-(3-(2-methyl-1- (2,2,2-trifluoroethyl)- 1H-indole-3- carboxamido)-4- (piperidin-1- yl)benzamido)phenyl) acetate | 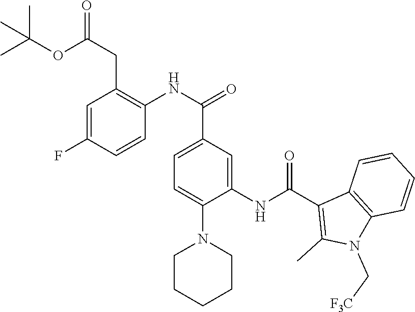 [SMILES: Cc1c(C(=O)Nc2cc(C(=O)Nc3ccc(F)cc3CC(=O)OC(C)(C)C)ccc2N2CCCCC2)c2ccccc2n1CC(F)(F)F] | UPLC-MS: Rₜ = 1.62 min. MS (ESI+): m/z = 667.8 [M + H]⁺. |
| 10ad | tert-butyl 2-(5-fluoro- 2-(3-(5-methyl-1- (2,2,2-trifluoroethyl)- 1H-indazole-3- carboxamido)-4- (piperidin-1- yl)benzamido)phenyl) acetate | 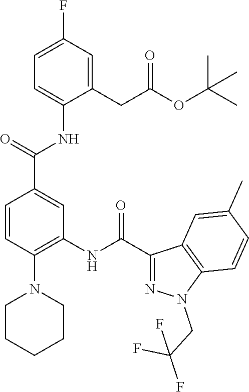 [SMILES: Cc1ccc2c(c1)c(C(=O)Nc1cc(C(=O)Nc3ccc(F)cc3CC(=O)OC(C)(C)C)ccc1N1CCCCC1)nn2CC(F)(F)F] | UPLC-MS: Rₜ = 1.70 min. MS (ESI+): m/z = 668.7 [M + H]⁺. prep-HPLC (Method described herein, Water (Acid): MeCN, gradient F) |
| 10ae | tert-butyl 2-(5-fluoro- 2-(3-(5-fluoro-1- (2,2,2-trifluoroethyl)- 1H-indazole-3- carboxamido)-4- (piperidin-1- yl)benzamido)phenyl) acetate | 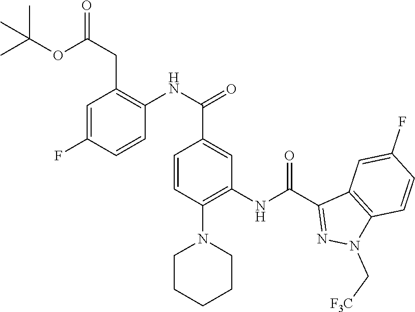 [SMILES: CC(C)(C)OC(=O)Cc1cc(F)ccc1NC(=O)c1ccc(N2CCCCC2)c(NC(=O)c2nn(CC(F)(F)F)c3ccc(F)cc23)c1] | ¹H NMR (400 MHz, DMSO-d6) δ: 9.98 (s, 1 H), 9.92 (s, 1 H), 8.94-9.02 (m, 1 H), 8.06-8.12 (m, 1 H), 7.92-7.99 (m, 1 H), 7.71-7.76 (m, 1 H), 7.53-7.62 (m, 1 H), 7.36-7.43 (m, 2 H), 7.13-7.23 (m, 2 H), 5.65-5.74 (m, 2 H), 3.67 (s, 2 H), 2.85- 2.93 (m, 4 H), 1.80 (quin, J = 5.39 Hz, 4 H), 1.57-1.64 (m, 2 H), 1.29 (s, 9 H) UPLC-MS: Rₜ = 1.60 min. MS (ESI+): m/z = 672.6 [M + H]⁺. |
| 10af | tert-butyl 2-(2-(3-(1- ((1,4-dioxan-2- yl)methyl)-1H- indazole-3- carboxamido)-4- (piperidin-1- yl)benzamido)-5- fluorophenyl)acetate | 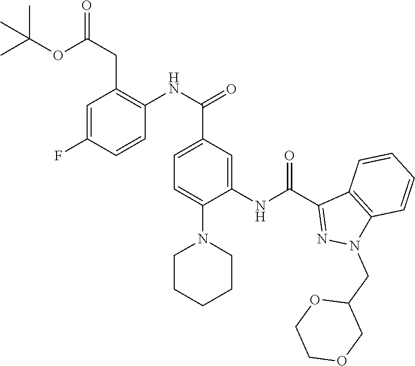 [SMILES: CC(C)(C)OC(=O)Cc1cc(F)ccc1NC(=O)c1ccc(N2CCCCC2)c(NC(=O)c2nn(CC3COCCO3)c3ccccc23)c1] | UPLC-MS: Rₜ = 1.55 min. MS (ESI+): m/z = 672.9 [M + H]⁺. |
| | | Method described | |
| | | herein | |
| 10ag | tert-butyl 2-(5-fluoro- 2-(3-(5-methoxy-1- (2,2,2-trifluoroethyl)- 1H-indazole-3- carboxamido)-4- (piperidin-1- yl)benzamido)phenyl) acetate | 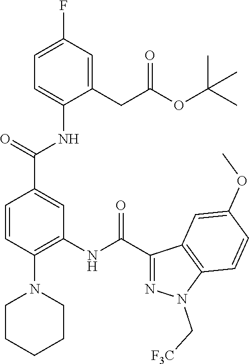 [SMILES: COc1ccc2c(c1)c(C(=O)Nc1cc(C(=O)Nc3ccc(F)cc3CC(=O)OC(C)(C)C)ccc1N1CCCCC1)nn2CC(F)(F)F] | ¹H NMR (400 MHz, DMSO-d6) δ: 9.96 (s, 1 H), 9.91 (s, 1 H), 8.98-9.03 (m, 1 H), 7.91 (d, J = 9.12 Hz, 1 H), 7.70-7.75 (m, 1 H), 7.64-7.69 (m, 1 H), 7.35- 7.43 (m, 2 H), 7.26 (dd, J = 9.25, 2.41 Hz, 1 H), 7.14-7.22 (m, 2 H), 5.57-5.70 (m, 2 H), 3.86 (s, 3 H), 3.67 (s, 2 H), 2.83-2.97 (m, 4 H), 1.74-1.89 (m, 4 H), 1.56-1.70 (m, 2 H), 1.29 (s, 9 H). UPLC-MS (method 6): Rₜ = 1.65 min. MS (ESI+): m/z = 684.8 [M + H]⁺. prep-HPLC (Method described herein, Water (Acid): MeCN, gradient F) |
| 10ah | tert-butyl 2-(5-fluoro- 2-(4-(piperidin-1-yl)- 3-(1-((tetrahydro-2H- pyran-3-yl)methyl)- 1H-indazole-3- carboxamido) benzamido) phenyl)acetate | 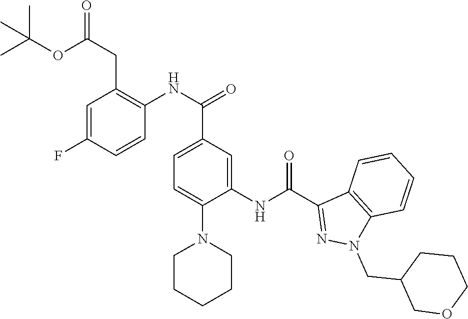 [SMILES: CC(C)(C)OC(=O)Cc1cc(F)ccc1NC(=O)c1ccc(N2CCCCC2)c(NC(=O)c2nn(CC3CCCOC3)c3ccccc23)c1] | UPLC-MS: Rₜ = 1.65 min. MS (ESI+): m/z = 671.0 [M + H]⁺. |
| 10ai | tert-butyl 2-(2-(3-(1- (1-cyclopropylethyl)- 1H-indazole-3- carboxamido)-4- (piperidin-1- yl)benzamido)-5- fluorophenyl)acetate | 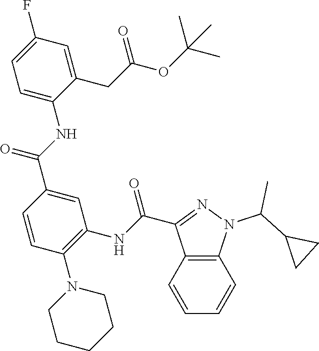 [SMILES: CC(C1CC1)n1nc(C(=O)Nc2cc(C(=O)Nc3ccc(F)cc3CC(=O)OC(C)(C)C)ccc2N2CCCCC2)c2ccccc21] | ¹H NMR (400 MHz, DMSO-d6) δ: 10.13 (s, 1 H), 9.92 (s, 1 H), 9.02-9.05 (m, 1 H), 8.24-8.33 (m, 1 H), 7.84-7.92 (m, 1 H), 7.69-7.74 (m, 1 H), 7.47-7.53 (m, 1 H), 7.33-7.43 (m, 3 H), 7.13-7.22 (m, 2 H), 4.39-4.48 (m, 1 H), 3.68 (s, 2 H), 2.88-2.95 (m, 4 H), 1.80-1.90 (m, 4 H), 1.71 (d, J = 6.84 Hz, 3 H), 1.59- 1.67 (m, 2 H), 1.39-1.51 (m, 1 H), 1.30 (s, 9 H), 0.62-0.72 (m, 1 H), 0.45-0.54 (m, 1 H), 0.31- 0.44 (m, 2 H). UPLC-MS: Rₜ = 1.75 min. MS (ESI+): m/z = 640.7 [M + H]⁺. prep-HPLC (Method described herein, Water (Acid): MeCN, gradient F) |
| | | Method described | |
| | | herein | |
| 10aj | tert-butyl 2-(5-fluoro- 2-(4-(piperidin-1-yl)- 3-(1-(1,1,1- trifluoropropan-2-yl)- 1H-indazole-3- carboxamido) benzamido) phenyl)acetate | 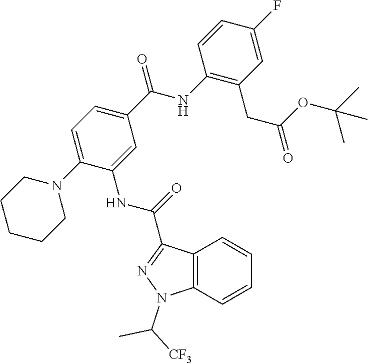 [SMILES: CC(n1nc(C(=O)Nc2cc(C(=O)Nc3ccc(F)cc3CC(=O)OC(C)(C)C)ccc2N2CCCCC2)c2ccccc21)C(F)(F)F] | UPLC-MS (method 3): Rₜ = 1.68 min. MS (ESI+): m/z = 668.8 [M + H]⁺. |
| 10ak | tert-butyl 2-(5-fluoro- 2-(3-(4-methyl-1- (2,2,2-trifluoroethyl)- 1H-indazole-3- carboxamido)-4- (piperidin-1- yl)benzamido)phenyl) acetate | 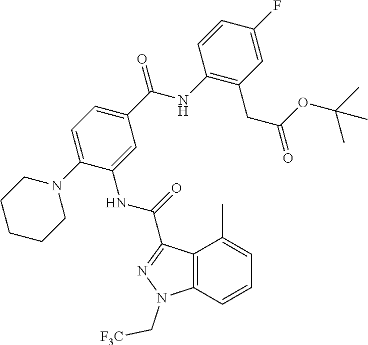 [SMILES: Cc1cccc2c1c(C(=O)Nc1cc(C(=O)Nc3ccc(F)cc3CC(=O)OC(C)(C)C)ccc1N1CCCCC1)nn2CC(F)(F)F] | UPLC-MS: Rₜ = 1.67 min. MS (ESI+): m/z = 668.7 [M + H]⁺. |
| 10al | tert-butyl (S)-2-(5- fluoro-2-(4- (piperidin-1-yl)-3-(1- ((tetrahydrofuran-2- yl)methyl)-1H- indazole-3- carboxamido) benzamido) phenyl)acetate | 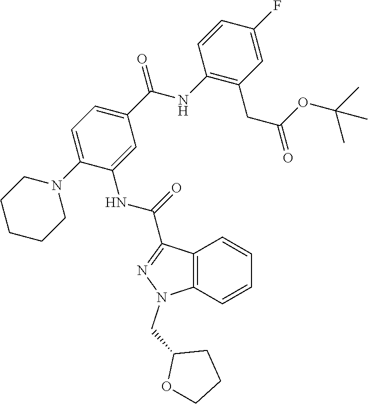 [SMILES: CC(C)(C)OC(=O)Cc1cc(F)ccc1NC(=O)c1ccc(N2CCCCC2)c(NC(=O)c2nn(C[C@@H]3CCCO3)c3ccccc23)c1] | UPLC-MS: Rₜ = 1.67 min. MS (ESI+): m/z = 656.7 [M + H]⁺. |
| | | Method described | |
| | | herein | |
| 10am | tert-butyl (R)-2-(5- fluoro-2-(4- (piperidin-1-yl)-3-(1- ((tetrahydrofuran-2- yl)methyl)-1H- indazole-3- carboxamido) benzamido) phenyl)acetate | 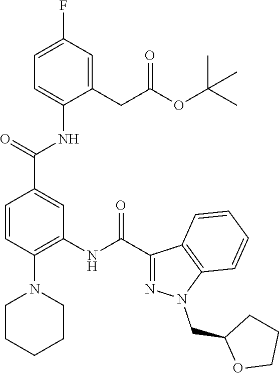 [SMILES: CC(C)(C)OC(=O)Cc1cc(F)ccc1NC(=O)c1ccc(N2CCCCC2)c(NC(=O)c2nn(C[C@H]3CCCO3)c3ccccc23)c1] | ¹H NMR (400 MHz, DMSO-d6) δ: 10.04 (s, 1 H), 9.91 (s, 1 H), 9.00 (d, J = 2.03 Hz, 1 H), 8.24- 8.27 (m, 1 H), 7.85-7.88 (m, 1 H), 7.71 (dd, J = 8.24, 2.15 Hz, 1 H), 7.49-7.54 (m, 1 H), 7.34- 7.43 (m, 3 H), 7.13-7.22 (m, 2 H), 4.61 (d, J = 5.83 Hz, 2 H), 4.34-4.45 (m, 1 H), 3.57-3.76 (m, 4 H), 2.85-2.96 (m, 4 H), 1.96-2.09 (m, 1 H), 1.72-1.89 (m, 7 H), 1.57-1.68 (m, 2 H), 1.29 (s, 9 H). UPLC-MS (method 6): Rₜ = 1.66 min. MS (ESI+): m/z = 656.8 [M + H]⁺. |
| 10an | tert-butyl 2-(5-fluoro- 2-(3-(6-methyl-1- (2,2,2-trifluoroethyl)- 1H-indazole-3- carboxamido)-4- (piperidin-1- yl)benzamido)phenyl) acetate | 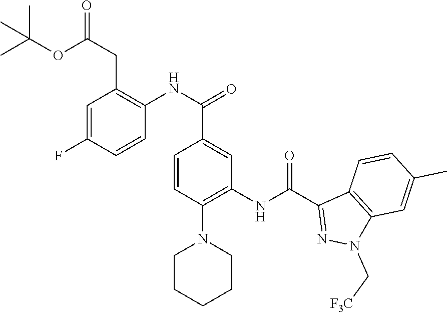 [SMILES: Cc1ccc2c(C(=O)Nc3cc(C(=O)Nc4ccc(F)cc4CC(=O)OC(C)(C)C)ccc3N3CCCCC3)nn(CC(F)(F)F)c2c1] | ¹H NMR (400 MHz, DMSO-d6) δ: 9.98 (s, 1 H), 9.91 (s, 1 H), 8.96-9.01 (m, 1 H), 8.15-8.20 (m, 1 H), 7.79 (br s, 1 H), 7.69- 7.76 (m, 1 H), 7.35-7.43 (m, 2 H), 7.26-7.29 (m, 1 H), 7.13- 7.22 (m, 2 H), 5.53-5.66 (m, 2 H), 3.67 (s, 2 H), 2.85-2.92 (m, 4 H), 2.50 (s, 3H, behind DMSO), 1.77-1.87 (m, 4 H), 1.57-1.65 (m, 2 H), 1.29 (s, 9 H). UPLC-MS: Rₜ = 1.65 min. MS (ESI+): m/z = 668.7 [M + H]⁺. |
| 10ao | tert-butyl 2-(5-fluoro- 2-(3-(7-methyl-1- (2,2,2-trifluoroethyl)- 1H-indazole-3- carboxamido)-4- (piperidin-1- yl)benzamido)phenyl) acetate | 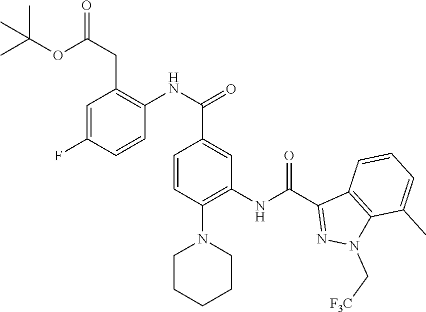 [SMILES: Cc1cccc2c(C(=O)Nc3cc(C(=O)Nc4ccc(F)cc4CC(=O)OC(C)(C)C)ccc3N3CCCCC3)nn(CC(F)(F)F)c12] | UPLC-MS: Rₜ = 1.68 min. MS (ESI+): m/z = 668.7 [M + H]⁺. |
| 10ap | tert-butyl 2-(5-fluoro- 2-(4-(piperidin-1-yl)- 3-(1-(2,2,2- trifluoroethyl)- 1,4,5,6- tetrahydrocyclopenta [c]pyrazole-3- carboxamido) benzamido) phenyl)acetate | 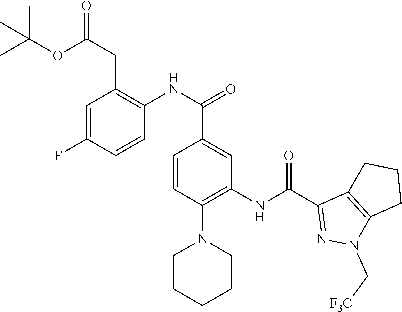 [SMILES: CC(C)(C)OC(=O)Cc1cc(F)ccc1NC(=O)c1ccc(N2CCCCC2)c(NC(=O)c2nn(CC(F)(F)F)c3c2CCC3)c1] | ¹H NMR (400 MHz, DMSO-d6) δ: 9.86 (s, 1 H), 9.67 (s, 1 H), 8.88 (d, J = 2.28 Hz, 1 H), 7.68 (dd, J = 8.36, 2.03 Hz, 1 H), 7.38 (dd, J = 8.74, 5.70 Hz, 1 H), 7.32 (d, J = 8.36 Hz, 1 H), 7.11-7.21 (m, 2 H), 5.11-5.25 (m, 2 H), 3.65 (s, 2 H), 2.72-2.88 (m, 8 H), 2.53-2.61 (m, 2 H), 1.70- 1.82 (m, 4 H), 1.52-1.64 (m, 2 H), 1.27 (m, 9 H). UPLC-MS: Rₜ = 1.58 min. MS (ESI+): m/z = 644.7 [M + H]⁺. |
| 10aq | tert-butyl 2-(5-fluoro- 2-(4-(piperidin-1-yl)- 3-(1-(1-(tetrahydro- 2H-pyran-4- yl)ethyl)-1H- indazole-3- carboxamido) benzamido) phenyl)acetate | 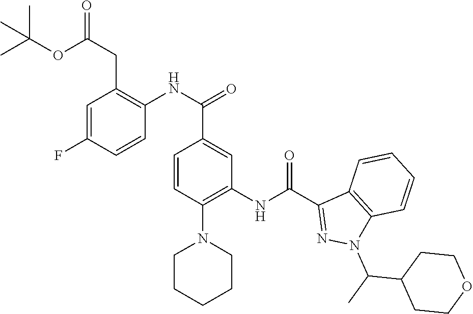 [SMILES: CC(C1CCOCC1)n1nc(C(=O)Nc2cc(C(=O)Nc3ccc(F)cc3CC(=O)OC(C)(C)C)ccc2N2CCCCC2)c2ccccc21] | UPLC-MS: Rₜ = 1.65 min. MS (ESI+): m/z = 684.7 [M + H]⁺. |
| 10ar | tert-butyl 2-(5-fluoro- 2-(N-methyl-4- (piperidin-1-yl)-3-(1- (2,2,2-trifluoroethyl)- 1H-indazole-3- carboxamido) benzamido) phenyl)acetate | 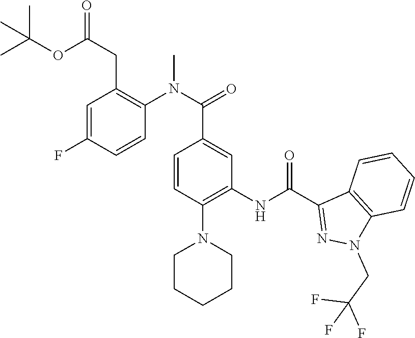 [SMILES: CN(C(=O)c1ccc(N2CCCCC2)c(NC(=O)c2nn(CC(F)(F)F)c3ccccc23)c1)c1ccc(F)cc1CC(=O)OC(C)(C)C] | ¹H NMR (400 MHz, DMSO-d6) δ: 9.74-10.15 (m, 1 H), 8.40- 8.69 (m, 1 H), 8.26-8.37 (m, 1 H), 7.94-8.05 (m, 1 H), 7.19- 7.67 (m, 4 H), 6.82-7.16 (m, 2 H), 6.80-6.90 (m, 1 H), 5.57- 5.73 (m, 2 H), 3.50-3.70 (m, 2 H), 3.22 (s, 3 H), 2.69-2.94 (m, 4 H), 1.68-1.86 (m, 4 H), 1.48- 1.66 (m, 2 H), 1.41 (s, 9 H). UPLC-MS: Rₜ = 1.64 min. MS (ESI+): m/z = 668.9 [M + H]⁺. |
| 10as | tert-butyl 2-(5-fluoro- 2-(3-(3-phenyl-1- (2,2,2-trifluoroethyl)- 1H-pyrazole-5- carboxamido)-4- (piperidin-1- yl)benzamido)phenyl) acetate | 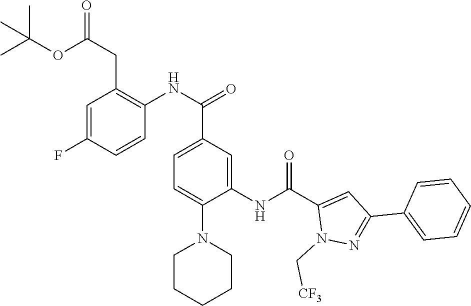 [SMILES: CC(C)(C)OC(=O)Cc1cc(F)ccc1NC(=O)c1ccc(N2CCCCC2)c(NC(=O)c2cc(-c3ccccc3)nn2CC(F)(F)F)c1] | ¹H NMR (400 MHz, DMSO-d₆) δ: 9.89 (s, 1 H), 9.78 (s, 1 H), 8.21-8.26 (m, 1 H), 7.80-7.88 (m, 3 H), 7.70-7.74 (m, 1 H), 7.48-7.55 (m, 2 H), 7.35-7.45 (m, 2 H), 7.24-7.28 (m, 1 H), 7.12-7.22 (m, 2 H), 5.56-5.69 (m, 2 H), 3.65 (s, 2 H), 2.88- 2.98 (m, 4 H), 1.61-1.72 (m, 4 H), 1.50-1.60 (m, 2 H), 1.28 (s, 9 H). UPLC-MS: Rₜ = 1.65 min. MS (ESI+): m/z = 680.8 [M + H]⁺. |
| 10at | tert-butyl 2-(5-fluoro- 2-(4-(piperidin-1-yl)- 3-(2-(2,2,2- trifluoroethyl)-2H- indazole-3- carboxamido) benzamido) phenyl)acetate | 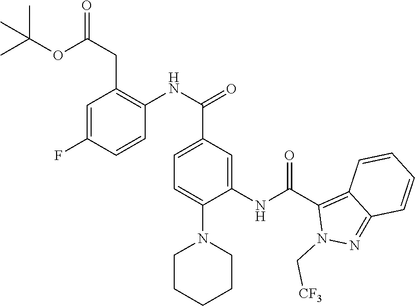 [SMILES: CC(C)(C)OC(=O)Cc1cc(F)ccc1NC(=O)c1ccc(N2CCCCC2)c(NC(=O)c2c3ccccc3nn2CC(F)(F)F)c1] | ¹H NMR (400 MHz, DMSO-d₆) δ: 9.96 (s, 1 H), 9.88 (s, 1 H), 8.69-8.75 (m, 1 H), 8.19-8.23 (m, 1 H), 7.88-7.95 (m, 1 H), 7.79-7.85 (m, 1 H), 7.42-7.54 (m, 3 H), 7.37-7.42 (m, 1 H), 7.13-7.23 (m, 2 H), 5.84-5.96 (m, 2 H), 3.66 (s, 2 H), 2.85- 2.96 (m, 4 H), 1.58-1.67 (m, 4 H), 1.48-1.56 (m, 2 H), 1.30 (s, 9 H). UPLC-MS: Rₜ = 1.61 min. MS (ESI+): m/z = 654.8 [M + H]⁺. |
| 10au | tert-butyl 2-(2-(3-(1- (cyclobutylmethyl)- 1H-indazole-3- carboxamido)-4- (piperidin-1- yl)benzamido)-5- fluorophenyl)acetate | 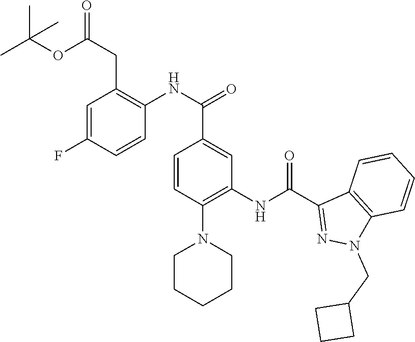 [SMILES: CC(C)(C)OC(=O)Cc1cc(F)ccc1NC(=O)c1ccc(N2CCCCC2)c(NC(=O)c2nn(CC3CCC3)c3ccccc23)c1] | UPLC-MS: Rₜ = 1.77 min. MS (ESI+): m/z = 640.8 [M + H]⁺. |
| 10av | tert-butyl 2-(5-fluoro- 2-(4-(piperidin-1-yl)- 3-(1-((tetrahydro-2H- pyran-4-yl)methyl)- 1H-indazole-3- carboxamido) benzamido) phenyl)acetate | 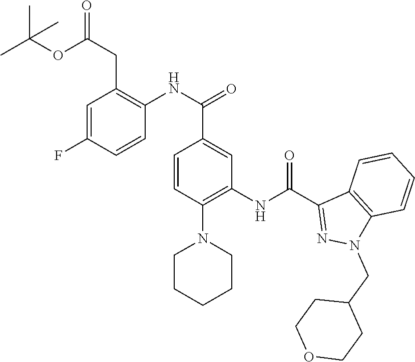 [SMILES: CC(C)(C)OC(=O)Cc1cc(F)ccc1NC(=O)c1ccc(N2CCCCC2)c(NC(=O)c2nn(CC3CCOCC3)c3ccccc23)c1] | UPLC-MS (method 6): Rₜ = 1.63 min. MS (ESI+): m/z = 671.0 [M + H]⁺. |

### Intermediate 11: Methyl 3-bromo-4-(1-piperidyl)benzoate

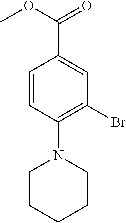 [SMILES: COC(=O)c1ccc(N2CCCCC2)c(Br)c1]

To a solution of methyl 3-bromo-4-fluoro-benzoate (1 g, 4.29 mmol, 1 eq) and piperidine (438.46 mg, 5.15 mmol, 1.2 eq) in DMF (10 mL) was added TEA (868.45 mg, 8.58 mmol, 1.19 mL, 2 eq). The mixture was stirred at 60° C. for 16 h. LCMS showed the reaction was completed and 54% desired mass was detected. The mixture was diluted with H₂O (30 mL) and extracted with EtOAc (30 mL×2). The combined organic layer was concentrated to afford a residue which was purified by column chromatography on silica (Petroleum ether: Ethyl acetate=100:1 to 20:1) to afford methyl 3-bromo-4-(1-piperidyl)benzoate (1.2 g, crude) as yellow oil. ¹H NMR (400 MHz, CDCl₃) δ=8.23 (d, J=2.1 Hz, 1H), 7.93 (dd, J=2.0, 8.4 Hz, 1H), 7.06-6.99 (m, 1H), 3.90 (s, 3H), 3.11-3.01 (m, 4H), 1.81-1.74 (m, 4H), 1.66-1.59 (m, 2H). MS (ESIpos): m/z=298.1, 300.1 [M+H]⁺.

### Intermediate 12a: 3-Bromo-4-(1-piperidyl)benzoic acid

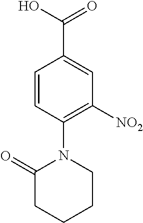 [SMILES: O=C(O)c1ccc(N2CCCCC2=O)c([N+](=O)[O-])c1]

To a solution of methyl 3-bromo-4-(1-piperidyl)benzoate (1.2 g, 4.02 mmol, 1 eq) in MeOH (8 mL) and H₂O (4 mL) was added NaOH (482.90 mg, 12.07 mmol, 3 eq). The mixture was stirred at 25° C. for 16 h. LCMS showed the reaction was completed and 51% desired mass was detected. The mixture was adjusted to pH=6 with 1M of HCl, then diluted with H₂O (20 mL) and extracted with EtOAc (20 mL×2). The combined organic layer was concentrated to afford 3-bromo-4-(1-piperidyl)benzoic acid (1 g, crude) as a white solid. MS (ESIpos): m/z=284.0, 286.0 [M+H]⁺.

### Intermediate 12b: 3-nitro-4-(2-oxopiperidin-1-yl)benzoic acid

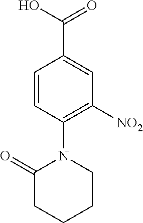 [SMILES: O=C(O)c1ccc(N2CCCCC2=O)c([N+](=O)[O-])c1]

To a solution of 4-fluoro-3-nitro-benzoic acid (1 g, 5.40 mmol, 1 eq) and piperidin-2-one (642.63 mg, 6.48 mmol, 1.2 eq) in THF (40 mL) was added NaH (540.17 mg, 13.51 mmol, 60% purity, 2.5 eq) at 0° C. The mixture was stirred at 25° C. for 1 hr. LCMS-S showed major of desired compound was detected. The reaction mixture was poured into 1M HCl solution (60 mL) and extracted with ethyl acetate 60 mL. The organic layer was washed with brine 60 mL, dried over Na₂SO₄, filtered, and concentrated under reduced pressure to give a residue. The residue was purified by reversed phase HPLC (0.1% FA condition) followed by lyophilization to afford the title compound (800 mg, 2.36 mmol, 43.71% yield, 78% purity) as a pale-yellow solid.

¹H NMR (400 MHz, CDCl₃) δ=8.81 (dd, J=2.1, 7.3 Hz, 1H), 8.65 (d, J=1.8 Hz, 1H), 8.32 (dd, J=1.8, 8.2 Hz, 1H), 3.80-3.75 (m, 2H), 2.60 (t, J=6.4 Hz, 2H), 2.11-2.06 (m, 2H), 2.01 (d, J=5.0 Hz, 2H).

LCMS-B; MS (ESIpos): m/z=265.2 [M+H]⁺.

### Intermediate 12c: 3-nitro-4-(2-oxooxazolidin-3-yl)benzoic acid

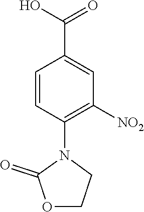 [SMILES: O=C(O)c1ccc(N2CCOC2=O)c([N+](=O)[O-])c1]

To a solution of 4-fluoro-3-nitro-benzoic acid (0.8 g, 4.32 mmol, 1 eq) and oxazolidin-2-one (451.59 mg, 5.19 mmol, 1.2 eq) in THF (20 mL) was added NaH (432.14 mg, 10.80 mmol, 60% purity, 2.5 eq) at 0° C. The mixture was stirred at 20° C. for 1 hr. The reaction mixture was poured into 1M HCl solution (60 mL) and extracted with ethyl acetate 60 mL. The organic layer was washed with brine 60 mL, dried over Na₃SO₄, filtered, and concentrated under reduced pressure to give a residue. The residue was purified by reversed phase HPLC (0.1% FA condition) followed by lyophilization to afford the title compound (430 mg, 1.62 mmol, 37.48% yield, 95% purity) as a yellow solid.

¹H NMR (400 MHz, DMSO-d₆) δ=13.62 (br s, 1H), 8.38 (d, J=1.8 Hz, 1H), 8.26 (dd, J=2.0, 8.4 Hz, 1H), 7.74 (d, J=8.6 Hz, 1H), 4.58-4.54 (m, 2H), 4.26-4.22 (m, 2H).

LCMS-B; MS (ESIpos): m/z=253.1 [M+H]⁺.

### Intermediate 13: Tert-butyl 2-12-113-bromo-4-(1-piperidyl)benzoyl]amino]-5-fluoro-phenyl]acetate

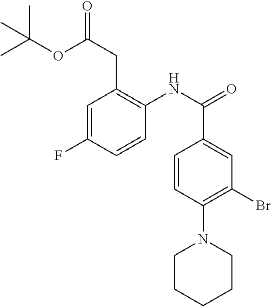 [SMILES: CC(C)(C)OC(=O)Cc1cc(F)ccc1NC(=O)c1ccc(N2CCCCC2)c(Br)c1]

To a solution of 3-bromo-4-(1-piperidyl)benzoic acid (950 mg, 3.34 mmol, 1 eq) in DMF (10 mL) were added HATU (1.91 g, 5.01 mmol, 1.5 eq), DIPEA (1.30 g, 10.03 mmol, 1.75 mL, 3 eq) followed by tert-butyl 2-(2-amino-5-fluoro-phenyl)acetate (828.42 mg, 3.68 mmol, 1.1 eq).

The mixture was stirred at 25° C. for 16 h. The mixture was diluted with H₂O (30 mL) and extracted with EtOAc (30 mL×2). The combined organic layer was concentrated to afford a residue which was purified by prep-HPLC (column: Phenomenex luna C18 150*40 mm*15 um; mobile phase: [water(FA)-ACN]; B %: 70%-100%, 10 min). The eluent was concentrated and freeze-dried to afford tert-butyl 2-[2-[[3-bromo-4-(1-piperidyl)benzoyl]amino]-5-fluoro-phenyl]acetate (800 mg, 1.47 mmol, 43.83% yield, 90% purity) as a brown solid. ¹H NMR (400 MHz, DMSO-d₆) δ=9.91 (s, 1H), 8.16 (d, J=2.0 Hz, 1H), 7.92 (dd, J=2.0, 8.4 Hz, 1H), 7.36 (dd, J=5.6, 8.7 Hz, 1H), 7.26-7.11 (m, 3H), 3.65 (s, 2H), 3.05-2.95 (m, 4H), 1.75-1.65 (m, 4H), 1.62-1.52 (m, 2H), 1.27 (s, 9H).

MS (ESIpos): m/z=491.1, 493.1 [M+H]⁺.

### Intermediate 14: 5-[[2-(2-Tert-butoxy-2-oxo-ethyl)-4-fluoro-phenyl]carbamoyl]-2-(1-piperidyl)benzoic acid

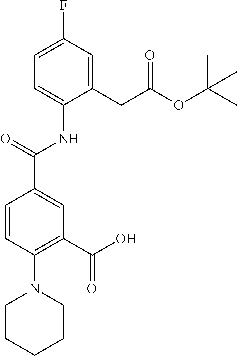 [SMILES: CC(C)(C)OC(=O)Cc1cc(F)ccc1NC(=O)c1ccc(N2CCCCC2)c(C(=O)O)c1]

To a solution of tert-butyl 2-[2-[[3-bromo-4(1-piperidyl)benzoyl]amino]-5-fluoro-phenyl]acetate (450 mg, 0.916 mmol, 1 eq) in DMF (9 mL) and H₂O (3 mL) were added Pd(OAc)₂ (10.28 mg, 0.0458 mmol, 0.05 eq), DPPP (18.89 mg, 0.0458 mmol, 0.05 eq) and TEA (185.33 mg, 1.83 mmol, 2 eq). The mixture was degassed under reduced pressure and purged with CO for three times. The mixture was stirred at 80° C. for 16 h under CO (45 Psi). The mixture was diluted with H₂O (10 mL) and extracted with EtOAc (10 mL×2). The combined organic layer was concentrated to afford 5-[[2-(2-tert-butoxy-2-oxo-ethyl)-4-fluoro-phenyl]carbamoyl]-2-(1-piperidyl)benzoic acid (200 mg, crude) as yellow oil. ¹H NMR (400 MHz, DMSO-d₆) δ=10.09 (s, 1H), 8.53 (d, J=2.1 Hz, 1H), 8.14 (dd, J=2.2, 8.4 Hz, 1H), 7.70 (d, J=8.5 Hz, 1H), 7.38 (dd, J=5.5, 8.6 Hz, 1H), 7.27-7.10 (m, 2H), 3.66 (s, 2H), 3.18-3.03 (m, 4H), 1.80-1.71 (m, 4H), 1.66-1.57 (m, 2H), 1.27 (s, 9H).

MS (ESIpos): m/z=457.2 [M+H]⁺.

### Intermediate 15a: 2-(3-amino-1H-indazol-1-yl)acetonitrile

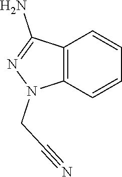 [SMILES: N#CCn1nc(N)c2ccccc21]

To a solution of commercially available 1H-indazol-3-amine (112 mg, 1.0 Eq, 841 μmol) in DMF (5 mL) was added NaH (40 mg, 60%/6 Wt, 1.2 Eq, 1.01 mmol) at 0° C. The resulting mixture was stirred at 0° C. for 30 min. 2-Chloroacetonitrile (69.9 mg, 1.1 Eq, 925 μmol) was added and the resulting mixture was stirred at 25° C. for 18 hours. Sat. NaHCO₃ solution and DCM were added, and the aqueous layer was extracted with DCM (3×). The combined organics were evaporated to dryness and purified via prep-HPLC (Method described herein, Water (Acid) MeCN, gradient B) to give the title compound (50 mg, 35%).

¹H NMR (500 MHz, DMSO-d₆) δ: 7.76 (d, 1H), 7.51 (d, 1H), 7.34-7.43 (m, 1H), 7.06 (m, 1H), 5.83 (s, 2H), 5.36 (s, 2H).

UPLC-MS: Rₜ=0.68 min; MS (ESI+): m/z=173.3 [M+H]⁺.

The following intermediates 15b and 15c were prepared in analogy to the intermediate 15a, using NaH or Cs₂CO₃ as a base:

| Int. | Name | Structure | Analytical Data |
|---|---|---|---|
| 15b | 1-(2,2,2- trifluoroethyl)- 1H- indazol-3- amine | 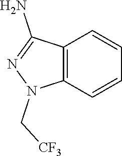 [SMILES: Nc1nn(CC(F)(F)F)c2ccccc12] | ¹H NMR (500 MHz, DMSO-d6) δ: 7.73 (d, 1H), 7.48 (d, 1H), 7.33 (m, 1H), 7.00 (m, 1H), 5.66 (s, 2H), 5.02 (m, 2H). UPLC-MS: Rₜ = 0.90 min; MS (ESI+): m/z = 216.4 [M + H]⁺. prep-HPLC (Method described herein, Water (Acid): MeCN, gradient C) |
| 15c | 1- ((tetrahydro- 2H-pyran-4- yl)methyl)- 1H-indazol- 3-amine | 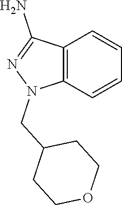 [SMILES: Nc1nn(CC2CCOCC2)c2ccccc12] | ¹H NMR (500 MHz, DMSO-d6) δ: 7.62-7.70 (m, 1H), 7.36 (d, 1H), 7.23 (m, 1H), 6.88 (m, 1H), 5.34-5.44 (m, 2H), 3.97 (d, 2H), 3.74-3.85 (m, 2H), 3.21 (m, 2H), 1.99-2.14 (m, 1H), 1.32-1.40 (m, 2H), 1.19-1.31 (m, 2H). UPLC-MS: Rₜ = 0.79 min; MS (ESI+): m/z = 232.4 [M + H]⁺. prep-HPLC (Method described herein, Water (Acid): MeCN, gradient C) |

### Intermediate 16a: tert-butyl 2-(2-(3-((1-(cyanomethyl)-1H-indazol-3-yl)carbamoyl)-4-(piperidin-1-yl)benzamido)-5-fluorophenyl)acetate

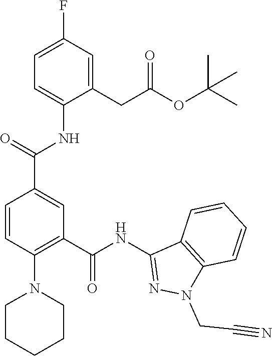 [SMILES: CC(C)(C)OC(=O)Cc1cc(F)ccc1NC(=O)c1ccc(N2CCCCC2)c(C(=O)Nc2nn(CC#N)c3ccccc23)c1]

A mixture of intermediate 14 (0.13 g, 1 Eq, 0.29 mmol), DIPEA (0.11 g, 0.15 mL, 3 Eq, 0.87 mmol), HATU (0.22 g, 2 Eq, 0.58 mmol) and DMF (2 mL) was stirred at 25° C. for 30 min. Intermediate 15a (50 mg, 1 Eq, 0.29 mmol) was added and the resulting mixture was stirred at 25° C. for 18 hours. DCM and water were added. The organic phase was extracted. The aqueous phase was washed with DCM. The organic phases were combined, dried over sodium sulfate, filtered and the solvent was removed under reduced pressure. Crude was purified by prep-HPLC (Method described herein, Water (Acidic): MeCN, gradient F): to give 50 mg (28%) of the title compound.

¹H NMR (400 MHz, DMSO-d₆) δ: 11.97 (s, 1H), 10.02 (s, 1H), 8.43-8.47 (m, 1H), 8.01-8.11 (m, 2H), 7.74-7.79 (m, 1H), 7.51-7.57(in, 1H), 7.35-7.43 (m, 2H), 7.12-7.27 (m, 3H), 5.77 (s, 2H), 3.66 (s, 2H), 3.07-3.17 (mn, 4H), 1.71-1.80 (mn, 4H), 1.51-1.62 (mn, 2H), 1.29 (m, 9H).

UPLC-MS: Rₜ=1.45 min; MS (ESI+): m/z=611.8 [M+H]⁺.

The following intermediates 16b and 16c were prepared in analogy to the intermediate 16a:

| Int. | Name | Structure | Analytical Data |
|---|---|---|---|
| 16b | tert-butyl 2-(5- fluoro-2-(4- (piperidin-1- yl)-3-((1- (2,2,2- trifluoroethyl)- 1H-indazol- 3- yl)carbamoyl) benzamido) phenyl)acetate | 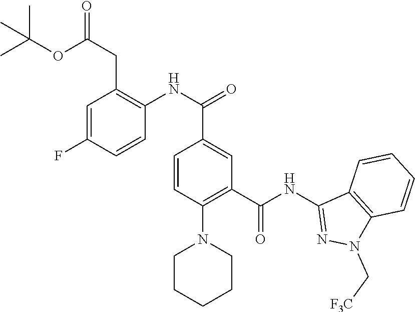 [SMILES: CC(C)(C)OC(=O)Cc1cc(F)ccc1NC(=O)c1ccc(N2CCCCC2)c(C(=O)Nc2nn(CC(F)(F)F)c3ccccc23)c1] | LCMS-A; MS (ESIpos): m/z = 654.2 [M + H]⁺. |
| 16c | tert-butyl 2-(5- fluoro-2-(4- (piperidin-1- yl)-3-((1- ((tetrahydro- 2H-pyran-4- yl)methyl)- 1H-indazol-3- yl)carbamoyl) benzamido) phenyl)acetate | 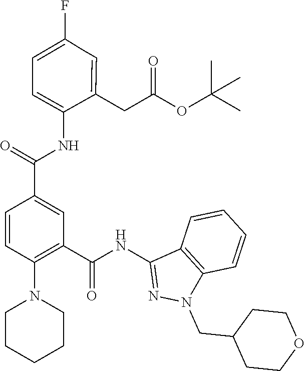 | ¹H NMR (400 MHz, DMSO-d6) δ: 12.01 (s, 1 H), 10.03 (s, 1 H), 8.45- 8.54 (m, 1 H), 8.00-8.11 (m, 2 H), 7.64-7.74 (m, 1 H), 7.34-7.46 (m, 3 H), 7.06-7.24 (m, 3 H), 4.22-4.32 (m, 2 H), 3.78-3.89 (m, 2 H), 3.66 (s, 2 H), 3.19-3.28 (m, 2 H), 3.05-3.14 (m, 4 H), 2.08-2.21 (m, 1 H), 1.71-1.83 (m, 4 H), 1.52-1.63 (m, 2 H), 1.39- 1.47 (m, 2 H), 1.30-1.39 (m, 2 H), 1.25-1.29 (m, 9 H). UPLC-MS: Rₜ = 1.55 min; MS (ESI+): m/z = 670.9 [M + H]⁺. |

### Intermediate 18: tert-butyl 2-(5-fluoro-2-(4-(piperidin-1-yl)-3-(3-(1-(2,2,2-trifluoroethyl)-1H-indazol-3-yl)ureido)benzamido)phenyl)acetate

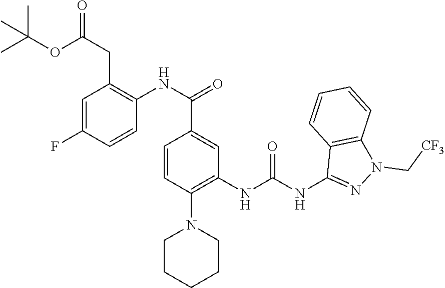 [SMILES: CC(C)(C)OC(=O)Cc1cc(F)ccc1NC(=O)c1ccc(N2CCCCC2)c(NC(=O)Nc2nn(CC(F)(F)F)c3ccccc23)c1]

To a solution of 4-nitrobenzyl chloroformate (67.5 mg, 1.1 Eq, 335 μmol) in DCM (4 mL) was added intermediate 6 (130 mg, 1 Eq, 304 μmol) (dissolved in DCM) dropwise. The resulting mixture was stirred at 25° C. for 17 hours. The mixture was evaporated to dryness. The obtained crude tert-butyl 2-(5-fluoro-2-(3-(((4-nitrophenoxy)carbonyl)amino)-4-(piperidin-1-yl)benzamido)phenyl)acetate (120 mg, 80% Wt, 1 Eq, 162 μmol), intermediate 15b (34.9 mg, 1 Eq, 162 μmol) and DCM (5 mL) were stirred at 60° C. for 18 hours. The mixture was evaporated to dryness. Crude was purified by prep-HPLC (Method described herein, Water (Acidic): MeCN, gradient F): to give 80 mg (74%) of the title compound.

¹H NMR (400 MHz, DMSO-d₆) δ: 10.29 (s, 1H), 9.80 (s, 1H), 9.33 (br s, 1H), 8.55-8.63 (m, 1H), 8.12 (d, J=8.11 Hz, 1H), 7.70-7.75 (m, 1H), 7.60-7.68 (m, 1H), 7.47-7.56 (m, 1H), 7.36-7.44 (m, 1H), 7.24 (d, J=8.36 Hz, 1H), 7.09-7.21 (m, 3H), 5.28-5.40 (m, 2H), 3.64 (s, 2H), 2.81-2.86 (m, 4H), 1.67-1.81 (m, 4H), 1.50-1.63 (m, 2H), 1.27 (s, 9H).

UPLC-MS: Rₜ=1.53 min; MS (ESI+): m/z=669.9 [M+H]⁺.

### Intermediate 19a: tert-butyl 2-(2-(4-(azetidin-1-yl)-3-nitrobenzamido)-5-fluorophenyl)acetate

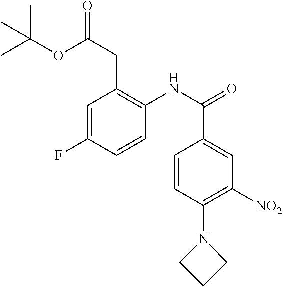 [SMILES: CC(C)(C)OC(=O)Cc1cc(F)ccc1NC(=O)c1ccc(N2CCC2)c([N+](=O)[O-])c1]

A mixture of intermediate 4 (400 mg, 1.02 mmol, 1 eq), azetidine (116.41 mg, 2.04 mmol, 2 eq) and DIPEA (263.52 mg, 2.04 mmol, 2 eq) in MeCN (4 mL) was stirred at 60° C. for 2 h under N₂ atmosphere. The reaction mixture was concentrated under reduced pressure to give a residue. The residue was purified by flash silica gel chromatography (Eluent of 0-20% Ethyl acetate/Petroleum ether) to afford the title compound (400 mg, 931.44 μmol, 91.36% yield, 100% purity) as a yellow solid.

¹H NMR (400 MHz, CDCh3) δ=9.76 (s, 1H), 8.52 (d, J=2.1 Hz, 1H), 8.05 (dd, J=2.2, 8.9 Hz, 1H), 7.95 (dd, J=5.4, 8.9 Hz, 1H), 7.05 (dt, J=2.9, 8.5 Hz, 1H), 6.96 (dd, J=2.9, 8.8 Hz, 1H), 6.66 (d, J=8.9 Hz, 1H), 4.12 (t, J=7.6 Hz, 4H), 3.58 (s, 2H), 2.51-2.38 (m, 2H), 1.49 (s, 9H).

LCMS-S; MS (ESIpos): m/z=452.0 [M+Na]

The following intermediates 19b-al were prepared in analogy to the intermediate 19a:

K₂CO as base was used instead of DIPEA for examples 19f-h.

| Int. | Name | Structure | Analytical Data |
|---|---|---|---|
| 19b | tert-butyl 2-(2- (4-(4,4- difluoropiperidin- 1-yl)-3- nitrobenzamido)- 5- fluorophenyl) acetate | 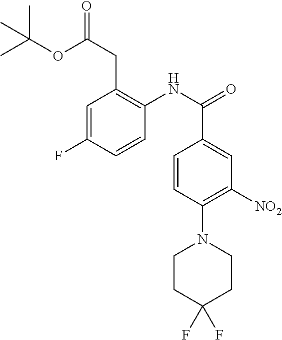 [SMILES: CC(C)(C)OC(=O)Cc1cc(F)ccc1NC(=O)c1ccc(N2CCC(F)(F)CC2)c([N+](=O)[O-])c1] | LCMS-S; MS (ESIpos): m/z 516.2 [M + Na]⁺. |
| 19c | tert-butyl 2-(2- (4-(4- cyanopiperidin- 1-yl)-3- nitrobenzamido)- 5- fluorophenyl) acetate | 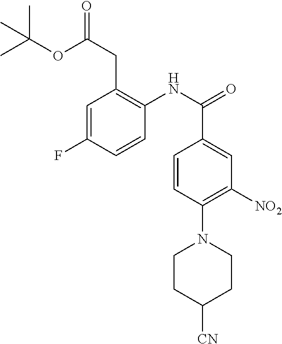 [SMILES: CC(C)(C)OC(=O)Cc1cc(F)ccc1NC(=O)c1ccc(N2CCC(C#N)CC2)c([N+](=O)[O-])c1] | LCMS-S; MS (ESIpos): m/z = 483.1 [M + H]⁺. |
| 19d | tert-butyl 2-(5- fluoro-2-(3- nitro-4-(2- azaspiro[3.3] heptan-2- yl)benzamido) phenyl)acetate | 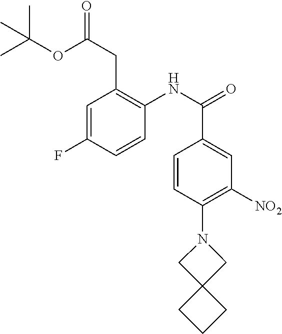 [SMILES: CC(C)(C)OC(=O)Cc1cc(F)ccc1NC(=O)c1ccc(N2CC3(CCC3)C2)c([N+](=O)[O-])c1] | ¹H NMR (400 MHz, CDCl₃) δ = 9.76 (s, 1H), 8.50 (d, J = 2.1 Hz, 1H), 8.04 (dd, J = 2.1, 8.8 Hz, 1H), 7.95 (dd, J = 5.4, 9.0 Hz, 1H), 7.08- 7.02 (m, 1H), 6.96 (dd, J = 2.9, 8.9 Hz, 1H), 6.63 (d, J = 8.9 Hz, 1H), 4.05 (s, 4H), 3.58 (s, 2H), 2.25 (t, J = 7.6 Hz, 4H), 1.92-1.83 (m, 2H), 1.49 (s, 9H). |
| 19e | tert-butyl 2-(5- fluoro-2-(3- nitro-4- (pyrrolidin-1- yl)benzamido) phenyl)acetate | 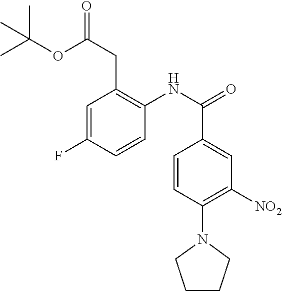 [SMILES: CC(C)(C)OC(=O)Cc1cc(F)ccc1NC(=O)c1ccc(N2CCCC2)c([N+](=O)[O-])c1] | ¹H NMR (400 MHz, CDCl₃) δ = 9.72 (s, 1H), 8.45 (d, J = 2.2 Hz, 1H), 8.04 (dd, J = 2.2, 9.0 Hz, 1H), 7.95 (dd, J = 5.4, 9.0 Hz, 1H), 7.08- 7.02 (m, 1H), 7.00-6.93 (m, 2H), 3.58 (s, 2H), 3.37-3.26 (m, 4H), 2.05-2.01 (m, 4H), 1.50 (s, 9H). |
| 19f | tert-butyl 2-(2- (4-(3- azabicyclo[3.1.0] hexan-3-yl)- 3- nitrobenzamido)- 5- fluorophenyl) acetate | 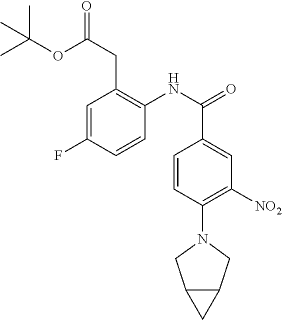 [SMILES: CC(C)(C)OC(=O)Cc1cc(F)ccc1NC(=O)c1ccc(N2CC3CC3C2)c([N+](=O)[O-])c1] | ¹H NMR (400 MHz, CDCl₃) δ = 9.72 (s, 1H), 8.33 (d, J = 2.3 Hz, 1H), 8.00 (dd, J = 2.4, 9.0 Hz, 1H), 7.95 (dd, J = 5.4, 8.9 Hz, 1H), 7.05 (dt, J = 3.0, 8.5 Hz, 1H), 6.99-6.94 (m, 2H), 3.59-3.51 (m, 4H), 3.47- 3.42 (m, 2H), 1.71-1.67 (m, 2H), 1.49 (s, 9H), 0.81-0.74 (m, 1H), 0.21 (q, J = 4.2 Hz, 1H). LCMS-B; MS (ESIpos): m/z = 456.3 [M + H]⁺. |
| 19g | tert-butyl 2-(5- fluoro-2-(3- nitro-4-(5- azaspiro[2.4] heptan-5- yl)benzamido) phenyl)acetate | 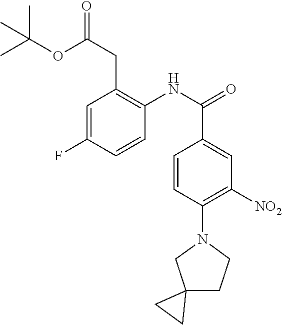 [SMILES: CC(C)(C)OC(=O)Cc1cc(F)ccc1NC(=O)c1ccc(N2CCC3(CC3)C2)c([N+](=O)[O-])c1] | ¹H NMR (400 MHz, CDCl₃) δ = 9.76 (s, 1H), 8.45 (d, J = 2.3 Hz, 1H), 8.06 (dd, J = 2.3, 9.0 Hz, 1H), 7.96 (dd, J = 5.4, 8.9 Hz, 1H), 7.06 (dt, J = 2.9, 8.6 Hz, 1H), 6.98-6.94 (m, 2H), 3.58 (s, 2H), 3.50 (t, J = 6.6 Hz, 2H), 3.19 (s, 2H), 1.94 (t, J = 6.7 Hz, 2H), 1.50 (s, 9H), 0.67 (d, J = 1.6 Hz, 2H), 0.66-0.65 (m, 2H). LCMS-B; MS (ESIpos): m/z = 470.3 [M + H]⁺. |
| 19h | tert-butyl 2-(2- (4-((1s,4s)-7- azabicyclo[2.2.1] heptan-7-yl)- 3- nitrobenzamido)- 5- fluorophenyl) acetate | 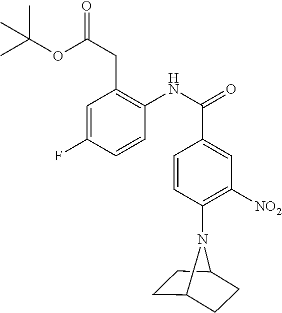 [SMILES: CC(C)(C)OC(=O)Cc1cc(F)ccc1NC(=O)c1ccc(N2C3CCC2CC3)c([N+](=O)[O-])c1] | ¹H NMR (400 MHz, CDCl₃) δ = 9.80 (s, 1H), 8.44 (d, J = 2.3 Hz, 1H), 8.04 (dd, J = 2.2, 8.8 Hz, 1H), 7.95 (dd, J = 5.4, 9.0 Hz, 1H), 7.09- 7.03 (m, 2H), 6.97 (dd, J = 2.8, 8.8 Hz, 1H), 4.11-4.07 (m, 2H), 3.58 (s, 2H), 1.93-1.86 (m, 4H), 1.54 (d, J = 7.1 Hz, 4H), 1.50 (s, 9H). LCMS-B; MS (ESIpos): m/z = 470.4 [M + H]⁺. |
| 19i | tert-butyl 2-(5- fluoro-2-(4- morpholino-3- nitrobenzamido) phenyl)acetate | 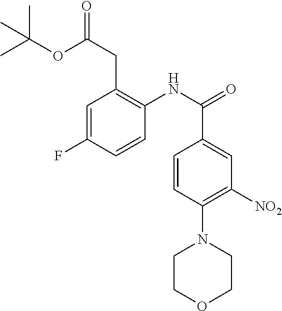 [SMILES: CC(C)(C)OC(=O)Cc1cc(F)ccc1NC(=O)c1ccc(N2CCOCC2)c([N+](=O)[O-])c1] | ¹H NMR (400 MHz, CDCl³) δ = 9.95 (br s, 1H), 8.51 (d, J = 2.1 Hz, 1H), 8.16 (dd, J = 2.1, 8.6 Hz, 1H), 7.97 (dd, J = 5.4, 8.9 Hz, 1H), 7.19 (d, J = 8.6 Hz, 1H), 7.10-7.03 (m, 1H), 6.97 (dd, J = 2.9, 8.9 Hz, 1H), 3.91-3.85 (m, 4H), 3.59 (s, 2H), 3.21-3.14 (m, 4H), 1.50 (s, 9H). |
| 19j | tert-butyl 2-(5- fluoro-2-(4-(4- methylpiperazin- 1-yl)-3- nitrobenzamido) phenyl)acetate | 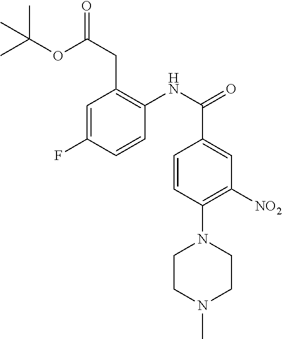 [SMILES: CN1CCN(c2ccc(C(=O)Nc3ccc(F)cc3CC(=O)OC(C)(C)C)cc2[N+](=O)[O-])CC1] | ¹H NMR (400 MHz, CDCl₃) δ = 9.91 (s, 1H), 8.49 (d, J = 2.3 Hz, 1H), 8.13 (dd, J = 2.3, 8.8 Hz, 1H), 7.96 (dd, J = 5.4, 8.9 Hz, 1H), 7.18 (d, J = 8.8 Hz, 1H), 7.10-7.03 (m, 1H), 6.97 (dd, J = 2.9, 8.9 Hz, 1H), 3.58 (s, 2H), 3.27-3.19 (m, 4H), 2.67-2.58 (m, 4H), 2.39 (s, 3H), 1.49 (s, 9H). |
| 19k | tert-butyl 2-(2- (4-(2- azabicyclo[2.2.1] heptan-2-yl)- 3- nitrobenzamido)- 5- fluorophenyl) acetate | 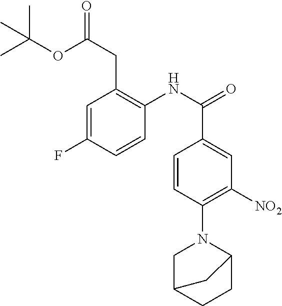 [SMILES: CC(C)(C)OC(=O)Cc1cc(F)ccc1NC(=O)c1ccc(N2CC3CCC2C3)c([N+](=O)[O-])c1] | ¹H NMR (400 MHz, DMSO-d₆) δ = 9.89 (s, 1H), 8.38 (d, J = 2.2 Hz, 1H), 7.99 (dd, J = 2.2, 9.0 Hz, 1H), 7.35 (dd, J = 5.6, 8.7 Hz, 1H), 7.24- 7.07 (m, 3H), 4.45 (s, 1H), 3.64 (s, 2H), 3.54-3.46 (m, 1H), 2.63 (s, 1H), 2.18 (d, J = 9.7 Hz, 1H), 1.81- 1.54 (m, 5H), 1.26 (s, 9H). LCMS-A; MS (ESIpos): m/z = 470.2 [M + H]⁺. |
| 19l | tert-butyl 2-(5- fluoro-2-(3- nitro-4-(2- oxopiperidin- 1- yl)benzamido) phenyl)acetate | 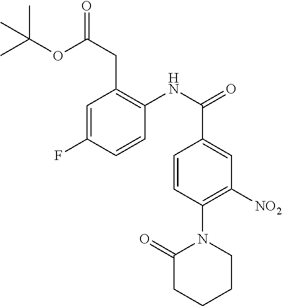 [SMILES: CC(C)(C)OC(=O)Cc1cc(F)ccc1NC(=O)c1ccc(N2CCCCC2=O)c([N+](=O)[O-])c1] | ¹H NMR (400 MHz, CDCl₃) δ = 10.21 (s, 1H), 8.65 (d, J = 2.0 Hz, 1H), 8.35 (dd, J = 2.0, 8.3 Hz, 1H), 7.99 (dd, J = 5.3, 8.9 Hz, 1H), 7.50 (d, J = 8.3 Hz, 1H), 7.09 (dt, J = 2.9, 8.5 Hz, 1H), 6.99 (dd, J = 2.9, 8.8 Hz, 1H), 3.77 (s, 2H), 3.60 (s, 2H), 2.55 (t, J = 6.5 Hz, 2H), 2.12-2.06 (m, 2H), 2.03-1.95 (m, 2H), 1.50 (s, 9H). LCMS-B; MS (ESIpos): m/z = 416.2 [M − 55]⁺. |
| 19m | tert-butyl 2-(5- fluoro-2-(3- nitro-4-(2- oxooxazolidin- 3- yl)benzamido) phenyl)acetate | 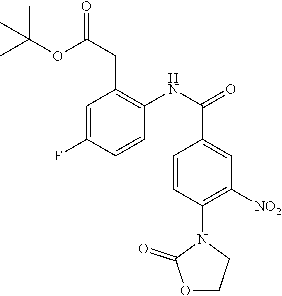 [SMILES: CC(C)(C)OC(=O)Cc1cc(F)ccc1NC(=O)c1ccc(N2CCOC2=O)c([N+](=O)[O-])c1] | LCMS-B; MS (ESIpos): m/z = 482.1 [M + Na]⁺. |
| 19n | tert-butyl 2-(2- (4-(4,4- dimethylpiperi- din-1-yl)-3- nitrobenzamido)- 5- fluorophenyl) acetate | 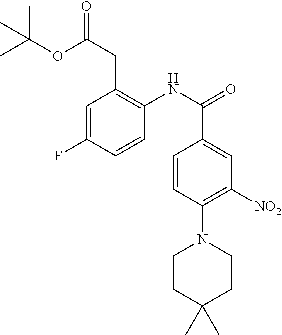 [SMILES: CC1(C)CCN(c2ccc(C(=O)Nc3ccc(F)cc3CC(=O)OC(C)(C)C)cc2[N+](=O)[O-])CC1] | ¹H NMR (400 MHz, DMSO-d₆) δ = 9.99 (s, 1H), 8.40 (d, J = 1.8 Hz, 1H), 8.08 (dd, J = 1.8, 8.7 Hz, 1H), 7.46-7.28 (m, 2H), 7.25-7.05 (m, 2H), 3.65 (s, 2H), 3.20-3.06 (m, 4H), 1,49-1.42 (m, 4H), 1.27 (s, 9H), 0.99 (s, 6H). LCMS-A; MS (ESIpos): m/z = 486.2 [M + H]⁺. |
| 19o | tert-butyl 2-(2- (4-(3,3- difluoropiperidin- 1-yl)-3- nitrobenzamido)- 5- fluorophenyl) acetate | 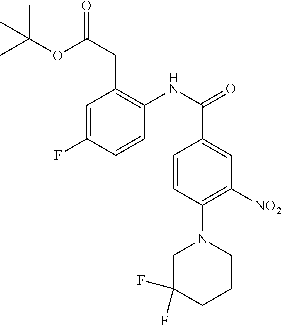 [SMILES: CC(C)(C)OC(=O)Cc1cc(F)ccc1NC(=O)c1ccc(N2CCCC(F)(F)C2)c([N+](=O)[O-])c1] | LCMS-B; MS (ESIpos): m/z = 494.2 [M + H]⁺. |
| 19p | tert-butyl 2-(5- fluoro-2-(3- nitro-4-(1,4- oxazepan-4- yl)benzamido) phenyl)acetate | 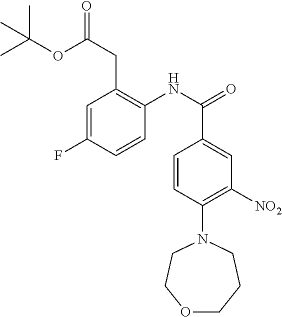 [SMILES: CC(C)(C)OC(=O)Cc1cc(F)ccc1NC(=O)c1ccc(N2CCCOCC2)c([N+](=O)[O-])c1] | LCMS-B; MS (ESIpos): m/z = 474.3 [M + H]⁺. |
| 19q | tert-butyl 2-(5- fluoro-2-(4-(4- methylpiperidin- 1-yl)-3- nitrobenzamido) phenyl)acetate | 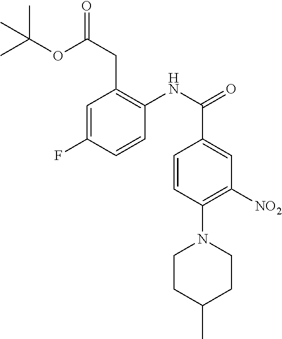 [SMILES: CC1CCN(c2ccc(C(=O)Nc3ccc(F)cc3CC(=O)OC(C)(C)C)cc2[N+](=O)[O-])CC1] | LCMS-B; MS (ESIpos): m/z = 472.3 [M + H]⁺. |
| 19r | tert-butyl 2-(5- fluoro-2-(4-(4- methoxypiperidin- 1-yl)-3- nitrobenzamido) phenyl)acetate | 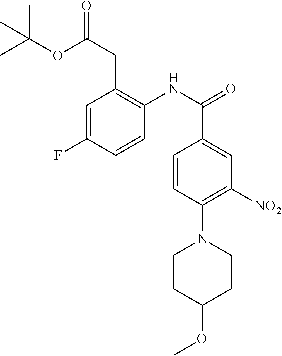 [SMILES: COC1CCN(c2ccc(C(=O)Nc3ccc(F)cc3CC(=O)OC(C)(C)C)cc2[N+](=O)[O-])CC1] | ¹H NMR (400 MHz, DMSO-d₆) δ = 10.00 (s, 1H), 8.41 (s, 1H), 8.09 (d, J = 8.4 Hz, 1H), 7.46-7.30 (m, 2H), 7.26-7.10 (m, 2H), 4.10-4.00 (m, 1H), 3.65 (s, 2H), 3.32-3.22 (m, 5H), 3.10-1.94 (m, 2H), 1.98-1.87 (m, 2H), 1.66-1.51 (m, 2H), 1.27 (s, 9H). LCMS-A; MS (ESIpos): m/z = 488.2 [M + H]⁺. |
| 19s | tert-butyl 2-(2- (4-(1,1- dioxidothio- morpholino)-3- nitrobenzamido)- 5- fluorophenyl) acetate | 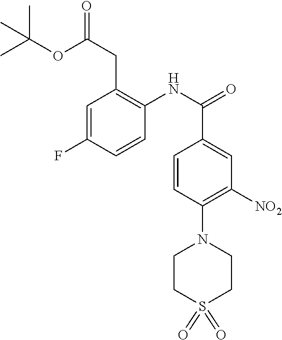 [SMILES: CC(C)(C)OC(=O)Cc1cc(F)ccc1NC(=O)c1ccc(N2CCS(=O)(=O)CC2)c([N+](=O)[O-])c1] | LCMS-B; MS (ESIpos): m/z = 508.2 [M + H]⁺. |
| 19t | tert-butyl 2-(5- fluoro-2-(4-(2- methylpiperidin- 1-yl)-3- nitrobenzamido) phenyl)acetate | 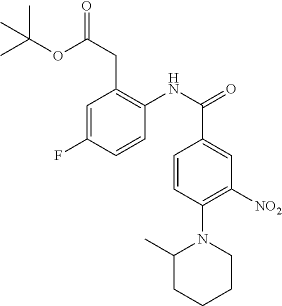 [SMILES: CC1CCCCN1c1ccc(C(=O)Nc2ccc(F)cc2CC(=O)OC(C)(C)C)cc1[N+](=O)[O-]] | ¹H NMR (400 MHz, CDCl₃) δ = 9.83 (s, 1H), 8.39 (d, J = 2.3 Hz, 1H), 8.09 (dd, J = 2.3, 8.8 Hz, 1H), 7.96 (dd, J = 5.4, 8.9 Hz, 1H), 7.23 (d, J = 8.8 Hz, 1H), 7.06 (dt, J = 2.9, 8.5 Hz, 1H), 6.97 (dd, J = 2.9, 8.8 Hz, 1H), 3.62 (d, J = 6.4 Hz, 1H), 3.59 (s, 2H), 3.33-3.25 (m, 1H), 2.92 (d, J = 12.3 Hz, 1H), 1.93- 1.86 (m, 1H), 1.76-1.66 (m, 3H), 1.66-1.60 (m, 1H), 1.52 (d, J = 4.5 Hz, 1H), 1.50 (s, 9H), 1.11 (d, J = 6.5 Hz, 3H). LCMS-B; MS (ESIpos): m/z = 472.3 [M + H]⁺. |
| 19u | tert-butyl 2-(2- (4-(2,6- dimethylpiperidin- 1-yl)-3- nitrobenzamido)- 5- fluorophenyl) acetate | 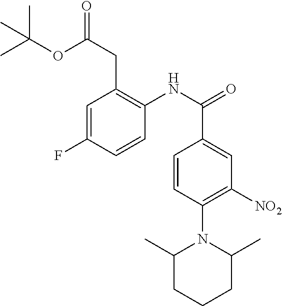 [SMILES: CC1CCCC(C)N1c1ccc(C(=O)Nc2ccc(F)cc2CC(=O)OC(C)(C)C)cc1[N+](=O)[O-]] | LCMS-B; MS (ESIpos): m/z = 486.3 [M + H]⁺. |
| 19v | tert-butyl 2-(5- fluoro-2-(4-(2- (hydroxymethyl) piperidin-1- yl)-3- nitrobenzamido) phenyl)acetate | 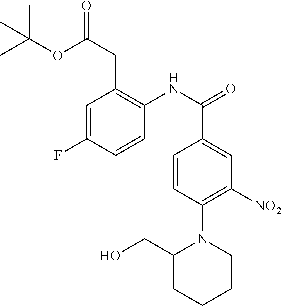 [SMILES: CC(C)(C)OC(=O)Cc1cc(F)ccc1NC(=O)c1ccc(N2CCCCC2CO)c([N+](=O)[O-])c1] | ¹H NMR (400 MHz, CDCl₃) δ = 9.88 (s, 1H), 8.45 (d, J = 2.3 Hz, 1H), 8.10 (dd, J = 2.2, 8.8 Hz, 1H), 7.97 (dd, J = 5.3, 9.0 Hz, 1H), 7.33- 7.29 (m, 1H), 7.07 (dt, J = 2.9, 8.5 Hz, 1H), 6.97 (dd, J = 2.8, 8.8 Hz, 1H), 4.10-4.04 (m, 2H), 3.58 (s, 2H), 3.51 (dd, J = 7.2, 9.9 Hz, 1H), 3.39-3.31 (m, 1H), 2.74-2.68 (m, 1H), 2.61-2.55 (m, 1H), 2.00-1.90 (m, 1H), 1.79-1.59 (m, 5H), 1.50 (s, 9H). LCMS-B; MS (ESIpos): m/z = 488.3 [M + H]⁺. |
| 19w | tert-butyl 2-(2- (4-(2- carbamoyl- piperidin-1-yl)-3- nitrobenzamido)- 5- fluorophenyl) acetate | 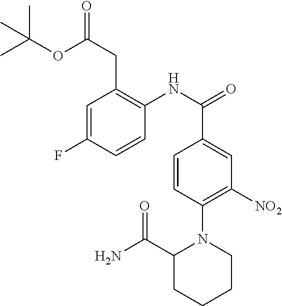 [SMILES: CC(C)(C)OC(=O)Cc1cc(F)ccc1NC(=O)c1ccc(N2CCCCC2C(N)=O)c([N+](=O)[O-])c1] | ¹H NMR (400 MHz, CDCl₃) δ = 10.00 (s, 1H), 8.49 (d, J = 2.1 Hz, 1H), 8.16 (dd, J = 2.1, 8.7 Hz, 1H), 7.97 (dd, J = 5.4, 8.9 Hz, 1H), 7.36- 7.29 (m, 2H), 7.07 (dt, J = 2.9, 8.5 Hz, 1H), 6.97 (dd, J = 2.8, 8.8 Hz, 1H), 5.51 (s, 1H), 4.30 (t, J = 4.6 Hz, 1H), 3.58 (s, 2H), 3.37-3.28 (m, 1H), 2.86-2.76 (m, 1H), 2.43-2.32 (m, 1H), 1.99-1.86 (m, 1H), 1.70- 1.56 (m, 4H), 1.50 (s, 9H). LCMS-T; MS (ESIpos): m/z = 501.3 [M + H]⁺. |
| 19x | | 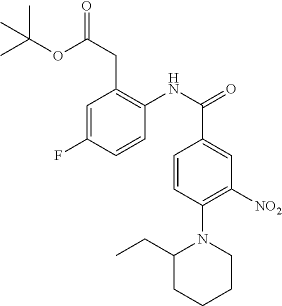 [SMILES: CCC1CCCCN1c1ccc(C(=O)Nc2ccc(F)cc2CC(=O)OC(C)(C)C)cc1[N+](=O)[O-]] | LCMS-R; MS (ESIpos): m/z = 486.3 [M + H]⁺. |
| 19y | tert-butyl 2-(5- fluoro-2-(4-(3- methylpiperidin- 1-yl)-3- nitrobenzamido) phenyl)acetate | 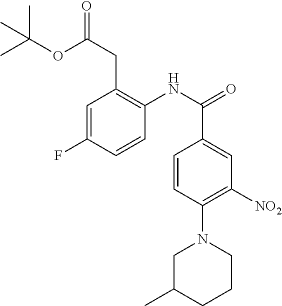 [SMILES: CC1CCCN(c2ccc(C(=O)Nc3ccc(F)cc3CC(=O)OC(C)(C)C)cc2[N+](=O)[O-])C1] | ¹H NMR (400 MHz, CDCl₃) δ = 9.74 (s, 1H), 8.39 (d, J = 2.2 Hz, 1H), 8.01 (dd, J = 2.2, 8.8 Hz, 1H), 7.88 (dd, J = 5.4, 8.9 Hz, 1H), 7.07 (d, J = 8.8 Hz, 1H), 7.02-6.94 (m, 1H), 6.89 (dd, J = 2.8, 8.8 Hz, 1H), 3.50 (s, 2H), 3.33-3.10 (m, 2H), 2.91-2.76 (m, 1H), 2.54 (dd, J = 10.4, 12.2 Hz, 1H), 1.86-1.73 (m, 2H), 1.72-1.61 (m, 2H), 1.42 (s, 9H), 1.12-0.99 (m, 1H), 0.86 (d, J = 6.5 Hz, 3H). LCMS-A; MS (ESIpos): m/z = 472.2 [M + H]⁺. |
| 19z | tert-butyl 2-(2- (4-(3,5- dimethylpiperi- din-1-yl)-3- nitrobenzamido)- 5- fluorophenyl) acetate | 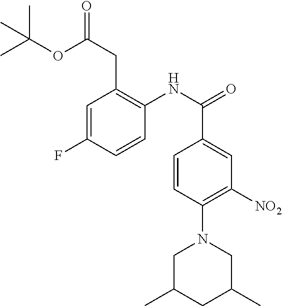 [SMILES: CC1CC(C)CN(c2ccc(C(=O)Nc3ccc(F)cc3CC(=O)OC(C)(C)C)cc2[N+](=O)[O-])C1] | ¹H NMR (400 MHz, CDCl₃) δ = 9.83 (s, 1H), 8.51-8.42 (m, 1H), 8.08 (dd, J = 2.3, 8.9 Hz, 1H), 7.96 (dd, J = 5.4, 8.9 Hz, 1H), 7.15 (d, J = 8.8 Hz, 1H), 7.06 (dt, J = 2.9, 8.5 Hz, 1H), 6.97 (dd, J = 2.9, 8.8 Hz, 1H), 3.58 (s, 2H), 3.33-3.12 (m, 2H), 2.86 (dd, J = 7.0, 12.4 Hz, 0.5H), 2.52 (t, J = 11.8 Hz, 1.6H), 2.15-2.08 (m, 0.5H), 1.94-1.82 (m, 2.5H), 1.50 (s, 9H), 1.02-0.88 (m, 6H), 0.83-0.72 (m, 1H). |
| 19aa | tert-butyl 2-(5- fluoro-2-(4-(4- hydroxypiperidin- 1-yl)-3- nitrobenzamido) phenyl)acetate | 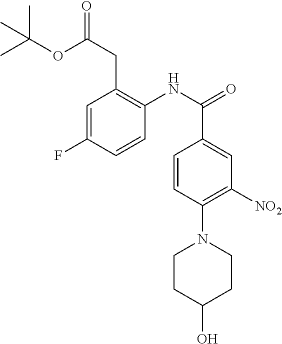 [SMILES: CC(C)(C)OC(=O)Cc1cc(F)ccc1NC(=O)c1ccc(N2CCC(O)CC2)c([N+](=O)[O-])c1] | LCMS-B; MS (ESIpos): m/z = 474.3 [M + H]⁺. |
| 19ab | tert-butyl (R)- 2-(5-fluoro-2- (4-(2- methylpiperidin- 1-yl)-3- nitrobenzamido) phenyl)acetate | 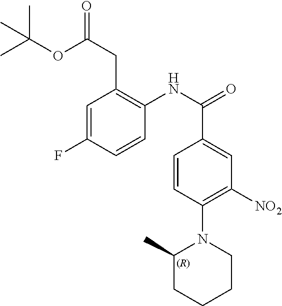 | LCMS-B; MS (ESIpos): m/z = 472.3 [M + H]⁺. |
| 19ac | tert-butyl (S)- 2-(5-fluoro-2- (4-(2- methylpiperidin- 1-yl)-3- nitrobenzamido) phenyl)acetate | 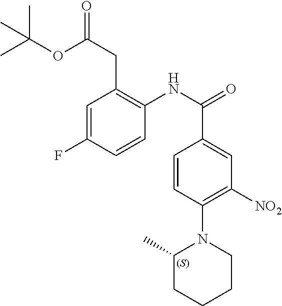 | LCMS-B; MS (ESIpos): m/z = 472.3 [M + H]⁺. |
| 19ad | tert-butyl (R)- 2-(5-fluoro-2- (4-(3- methylpiperidin- 1-yl)-3- nitrobenzamido) phenyl)acetate | 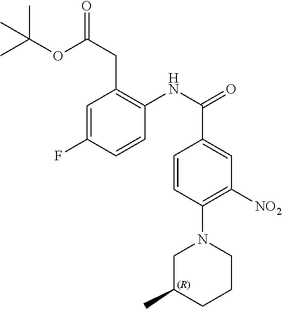 | ¹H NMR (400 MHz, CDCl₃) δ = 9.83 (s, 1H), 8.47 (d, J = 2.3 Hz, 1H), 8.09 (dd, J = 2.2, 8.8 Hz, 1H), 7.97 (dd, J = 5.4, 8.9 Hz, 1H), 7.15 (d, J = 8.9 Hz, 1H), 7.06 (d, J = 3.0 Hz, 1H), 6.97 (dd, J = 2.9, 8.9 Hz, 1H), 3.58 (s, 2H), 3.37-3.25 (m, 2H), 3.02-2.84 (m, 1H), 2.62 (dd, J = 10.4, 12.2 Hz, 1H), 1.89-1.84 (m, 1H), 1.81-1.71 (m, 2H), 1.50 (s, 9H), 1.37-1.19 (m, 2H), 0.94 (d, J = 6.4 Hz, 3H). LCMS-B; MS (ESIpos): m/z = 472.3 [M + H]⁺. |
| 19ae | tert-butyl (S)- 2-(5-fluoro-2- (4-(3- methylpiperidin- 1-yl)-3- nitrobenzamido) phenyl)acetate | 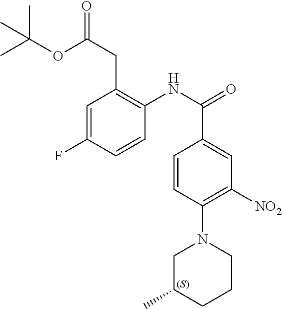 | LCMS-B; MS (ESIpos): m/z = 472.3 [M + H]⁺. |
| 19af | tert-butyl 2-(2- (4-(3- (difluoromethyl) piperidin-1- yl)-3- nitrobenzamido)- 5- fluorophenyl) acetate | 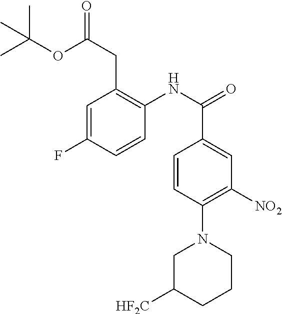 | LCMS-B; MS (ESIpos): m/z = 508.3 [M + H]⁺. |
| 19ag | tert-butyl 2-(2- (4-(4- (difluoromethyl) piperidin-1- yl)-3- nitrobenzamido)- 5- fluorophenyl) acetate | 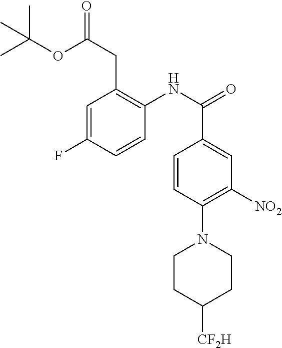 [SMILES: CC(C)(C)OC(=O)Cc1cc(F)ccc1NC(=O)c1ccc(N2CCC(C(F)F)CC2)c([N+](=O)[O-])c1] | LCMS-B; MS (ESIpos): m/z = 508.3 [M + H]⁺. |
| 19ah | tert-butyl 2-(5- fluoro-2-(4-(3- methoxypiperidin)- 1-yl)-3- nitrobenzamido) phenyl)acetate | 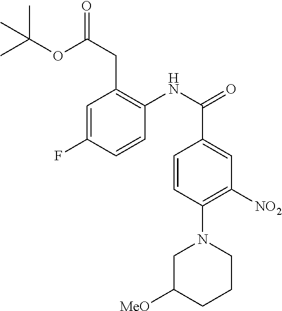 [SMILES: COC1CCCN(c2ccc(C(=O)Nc3ccc(F)cc3CC(=O)OC(C)(C)C)cc2[N+](=O)[O-])C1] | LCMS-B; MS (ESIpos): m/z = 488.3 [M + H]⁺. |
| 19ai | tert-butyl 2-(5- fluoro-2-(4-(3- hydroxypiperidin- 1-yl)-3- nitrobenzamido) phenyl)acetate | 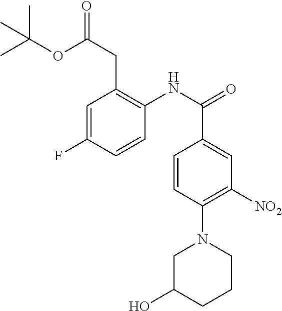 [SMILES: CC(C)(C)OC(=O)Cc1cc(F)ccc1NC(=O)c1ccc(N2CCCC(O)C2)c([N+](=O)[O-])c1] | LCMS-B; MS (ESIpos): m/z = 474.2 [M + H]⁺. |
| 19aj | tert-butyl 2-(5- fluoro-2-(4-(3- (hydroxymethyl) piperidin-1- yl)-3- nitrobenzamido) phenyl)acetate | 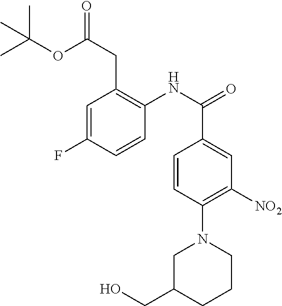 [SMILES: CC(C)(C)OC(=O)Cc1cc(F)ccc1NC(=O)c1ccc(N2CCCC(CO)C2)c([N+](=O)[O-])c1] | LCMS-B; MS (ESIpos): m/z = 488.3 [M + H]⁺. |
| 19ak | tert-butyl 2-(2- (4-(3- carbamoylpiper- idin-1-yl)-3- nitrobenzamido)- 5- fluorophenyl) acetate | 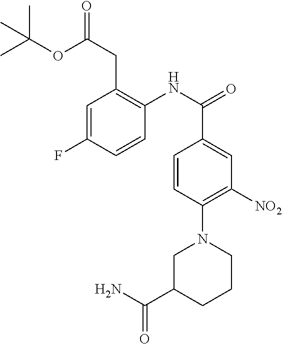 [SMILES: CC(C)(C)OC(=O)Cc1cc(F)ccc1NC(=O)c1ccc(N2CCCC(C(N)=O)C2)c([N+](=O)[O-])c1] | LCMS-B; MS (ESIpos): m/z = 501.3 [M + H]⁺. |
| 19al | tert-butyl 2-(2- (4-(4- ethylpiperidin- 1-yl)-3- nitrobenzamido)- 5- fluorophenyl) acetate | 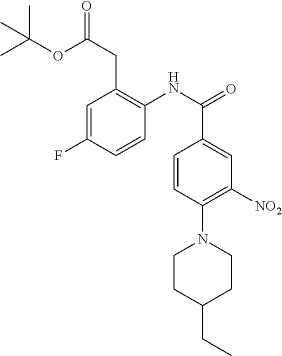 [SMILES: CCC1CCN(c2ccc(C(=O)Nc3ccc(F)cc3CC(=O)OC(C)(C)C)cc2[N+](=O)[O-])CC1] | ¹H NMR (400 MHz, CDCl₃) δ = 9.84 (s, 1H), 8.47 (d, J = 2.2 Hz, 1H), 8.09 (dd, J = 2.3, 8.8 Hz, 1H), 7.96 (dd, J = 5.4, 8.9 Hz, 1H), 7.15 (d, J = 8.8 Hz, 1H), 7.06 (dt, J = 2.9, 8.5 Hz, 1H), 6.97 (dd, J = 2.9, 8.8 Hz, 1H), 3.58 (s, 2H), 3.40 (d, J = 12.1 Hz, 2H), 2.96 (t, J = 11.9 Hz, 2H), 1.84-1.76 (m, 2H), 1.50 (s, 9H), 1.42-1.31 (m, 5H), 0.98-0.89 (m, 3H). |
| 19am | tert-butyl 2-(5- fluoro-2-(3- nitro-4-(6- azaspiro[2.5] octan-6- yl)benzamido) phenyl)acetate | 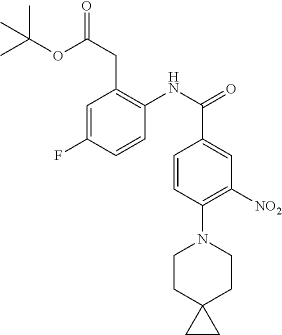 [SMILES: CC(C)(C)OC(=O)Cc1cc(F)ccc1NC(=O)c1ccc(N2CCC3(CC2)CC3)c([N+](=O)[O-])c1] | LCMS-B; MS (ESIpos): m/z = 484.2 [M + H]⁺. |
| 19an | tert-butyl (S)- 2-(2-(4-(2- ethylpiperidin- 1-yl)-3- nitrobenzamido)- 5- fluorophenyl) acetate | 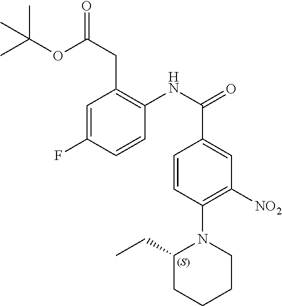 | LCMS-B; MS (ESIpos): m/z = 486.2 [M + H]⁺. |

### Intermediate 20a: tert-butyl 2-(2-(3-amino-4-(azetidin-1-yl)benzamido)-5-fluorophenyl)acetate

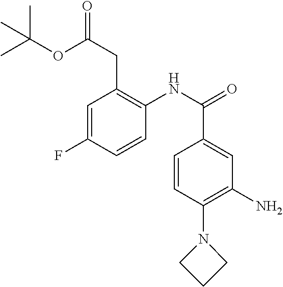 [SMILES: CC(C)(C)OC(=O)Cc1cc(F)ccc1NC(=O)c1ccc(N2CCC2)c(N)c1]

A mixture of intermediate 19a (200 mg, 465.72 μmol, 1 eq) and Pd/C (50 mg, 10% purity) in EtOAc (20 mL) was degassed and purged with H₂ for 2 times, and then the mixture was stirred at 25° C. for 0.5 hr under H₂ atmosphere (15 psi). LCMS-S showed ˜99% of desired compound was detected. The reaction mixture was filtered and concentrated under reduced pressure to give the title compound (180 mg, crude) as a white solid.

LCMS-S; MS (ESIpos): m/z=400.0 [M+H]⁺.

The following intermediates 20b-e, i-a1 were prepared in analogy to the intermediate 20a:

| Int. | Name | Structure | Analytical Data |
|---|---|---|---|
| 20b | tert-butyl 2-(2-(3- amino-4-(4,4- difluoropiperidin-1- yl)benzamido)-5- fluorophenyl)acetate | 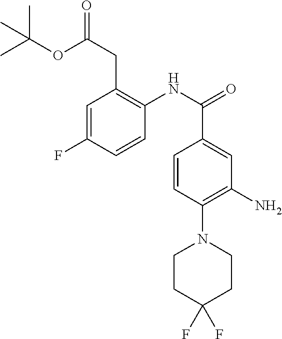 [SMILES: CC(C)(C)OC(=O)Cc1cc(F)ccc1NC(=O)c1ccc(N2CCC(F)(F)CC2)c(N)c1] | LCMS-S; MS (EPIpos): m/z = 464.1 [M + H]⁺. |
| 20c | tert-butyl 2-(2-(3- amino-4-(4- cyanopiperidin-1- yl)benzamido)-5- fluorophenyl)acetate | 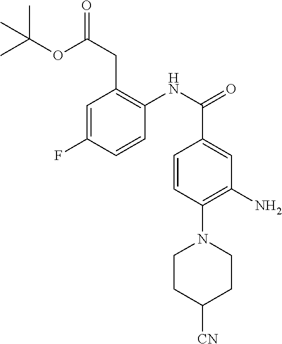 [SMILES: CC(C)(C)OC(=O)Cc1cc(F)ccc1NC(=O)c1ccc(N2CCC(C#N)CC2)c(N)c1] | ¹H NMR (400 MHz, CDCl₃) δ = 9.43 (s, 1H), 7.92 (dd, J = 5.4, 8.9 Hz, 1H), 7.42 (d, J = 2.1 Hz, 1H), 7.36 (dd, J = 2.0, 8.3 Hz, 1H), 7.09-7.02 (m, 2H), 6.96 (dd, J = 2.9, 8.9 Hz, 1H), 4.02 (br s, 2H), 3.56 (s, 2H), 3.23-3.14 (m, 2H), 2.97- 2.76 (m, 3H), 2.17-2.06 (m, 3H), 2.05-1.99 (m, 1H), 1.48 (s, 9H). LCMS-S; MS (ESIpos): m/z = 453.1 [M + H]⁺. |
| 20d | tert-butyl 2-(2-(3- amino-4-(2- azaspiro[3.3]heptan- 2-yl)benzamido)-5- fluorophenyl)acetate | 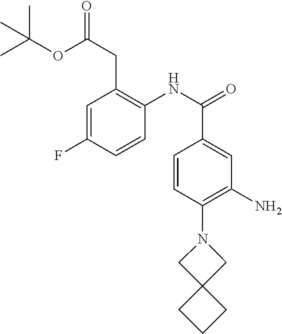 [SMILES: CC(C)(C)OC(=O)Cc1cc(F)ccc1NC(=O)c1ccc(N2CC3(CCC3)C2)c(N)c1] | LCMS-J; MS (ESIpos): m/z = 440.2 [M + H]⁺. |
| 20e | tert-butyl 2-(2-(3- amino-4-(pyrrolidin- 1-yl)benzamido)-5- fluorophenyl)acetate | 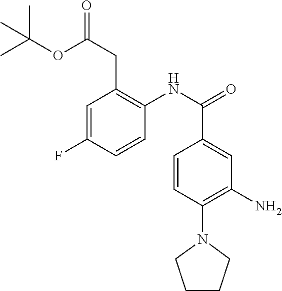 [SMILES: CC(C)(C)OC(=O)Cc1cc(F)ccc1NC(=O)c1ccc(N2CCCC2)c(N)c1] | LCMS-I; MS (ESIpos): m/z = 414.3 [M + H]⁺. |
| 20i | tert-butyl 2-(2-(3- amino-4- morpholinobenzamido)-5- fluorophenyl)acetate | 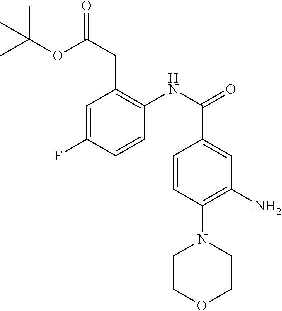 [SMILES: CC(C)(C)OC(=O)Cc1cc(F)ccc1NC(=O)c1ccc(N2CCOCC2)c(N)c1] | LCMS-AH; MS (ESIpos): m/z = 430.2 [M + H]⁺. |
| 20j | tert-butyl 2-(2-(3- amino-4-(4- methylpiperazin-1- yl)benzamido)-5- fluorophenyl)acetate | 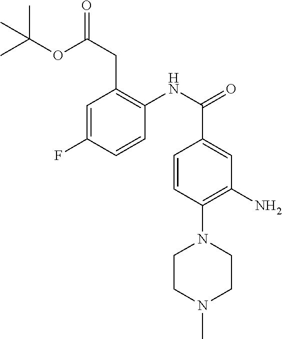 [SMILES: CN1CCN(c2ccc(C(=O)Nc3ccc(F)cc3CC(=O)OC(C)(C)C)cc2N)CC1] | LCMS-AH; MS (ESIpos): m/z = 443.3 [M + H]⁺. |
| 20k | tert-butyl 2-(2-(3- amino-4-(2- azabicyclo[2.2.1]heptan- 2-yl)benzamido)-5- fluorophenyl)acetate | 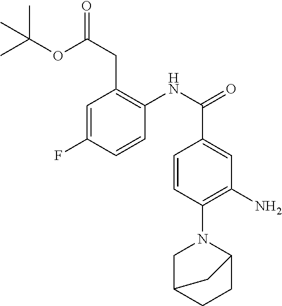 [SMILES: CC(C)(C)OC(=O)Cc1cc(F)ccc1NC(=O)c1ccc(N2CC3CCC2C3)c(N)c1] | LCMS-A; MS (ESIpos): m/z = 440.2 [M + H]⁺. |
| 20l | tert-butyl 2-(2-(3- amino-4-(2- oxopiperidin-1- yl)benzamido)-5- fluorophenyl)acetate | 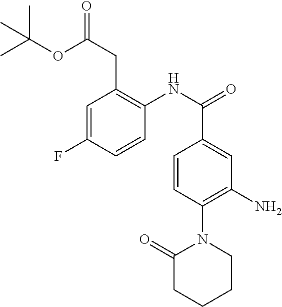 [SMILES: CC(C)(C)OC(=O)Cc1cc(F)ccc1NC(=O)c1ccc(N2CCCCC2=O)c(N)c1] | LCMS-S; MS (ESIpos): m/z = 385.9 [M − 55]⁺. |
| 20m | tert-butyl 2-(2-(3- amino-4-(2- oxooxazolidin-3- yl)benzamido)-5- fluorophenyl)acetate | 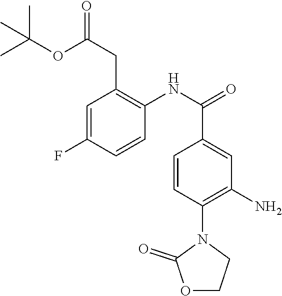 [SMILES: CC(C)(C)OC(=O)Cc1cc(F)ccc1NC(=O)c1ccc(N2CCOC2=O)c(N)c1] | LCMS-B; MS (ESIpos): m/z = 374.2 [M − 55]⁺. |
| 20n | tert-butyl 2-(2-(3- amino-4-(4,4- dimethylpiperidin-1- yl)benzamido)-5- fluorophenyl)acetate | 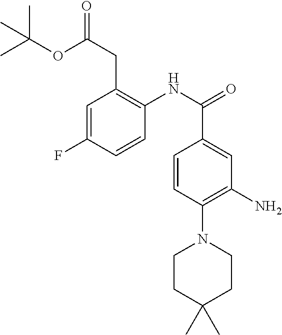 [SMILES: CC1(C)CCN(c2ccc(C(=O)Nc3ccc(F)cc3CC(=O)OC(C)(C)C)cc2N)CC1] | LCMS-A; MS (ESIpos): m/z = 456.3 [M + H]⁺. |
| 20o | tert-butyl 2-(2-(3- amino-4-(3,3- difluoropiperidin-1- yl)benzamido)-5- fluorophenyl)acetate | 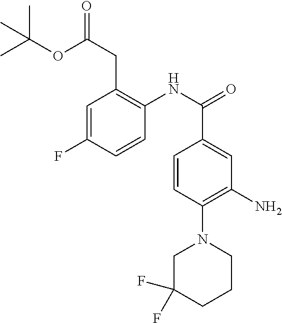 [SMILES: CC(C)(C)OC(=O)Cc1cc(F)ccc1NC(=O)c1ccc(N2CCCC(F)(F)C2)c(N)c1] | LCMS-S; MS (ESIpos): m/z = 464.1 [M + H]⁺. |
| 20p | tert-butyl 2-(2-(3- amino-4-(1,4- oxazepan-4- yl)benzamido)-5- fluorophenyl)acetate | 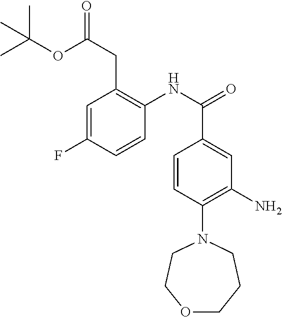 [SMILES: CC(C)(C)OC(=O)Cc1cc(F)ccc1NC(=O)c1ccc(N2CCCOCC2)c(N)c1] | LCMS-B; MS (ESIpos): m/z = 444.3 [M + H]⁺. |
| 20q | tert-butyl 2-(2-(3- amino-4-(4- methylpiperidin-1- yl)benzamido)-5- fluorophenyl)acetate | 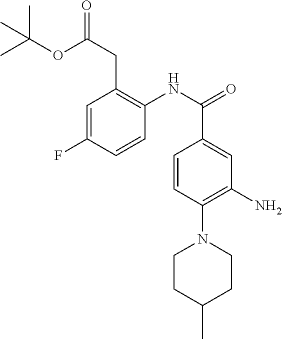 [SMILES: CC1CCN(c2ccc(C(=O)Nc3ccc(F)cc3CC(=O)OC(C)(C)C)cc2N)CC1] | LCMS-B; MS (ESIpos): m/z = 442.3 [M + H]⁺. |
| 20r | tert-butyl 2-(2-(3- amino-4-(4- methoxypiperidin-1- yl)benzamido)-5- fluorophenyl)acetate | 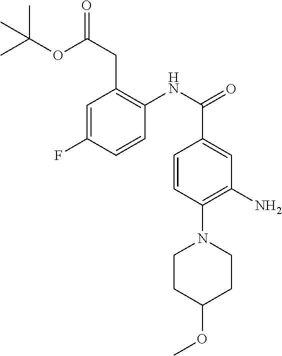 [SMILES: COC1CCN(c2ccc(C(=O)Nc3ccc(F)cc3CC(=O)OC(C)(C)C)cc2N)CC1] | LCMS-A; MS (ESIpos): m/z = 458.2 [M + H]⁺. |
| 20s | tert-butyl 2-(2-(3- amino-4-(1,1- dioxidothiomorpholino) benzamido)-5- fluorophenyl)acetate | 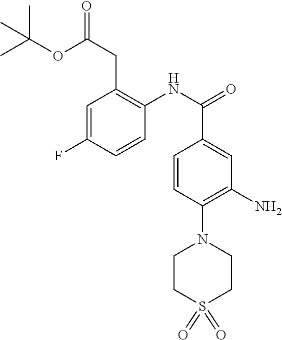 [SMILES: CC(C)(C)OC(=O)Cc1cc(F)ccc1NC(=O)c1ccc(N2CCS(=O)(=O)CC2)c(N)c1] | LCMS-B; MS (ESIpos): m/z = 422.2 [M − 55]⁺. |
| 20t | tert-butyl 2-(2-(3- amino-4-(2- methylpiperidin-1- yl)benzamido)-5- fluorophenyl)acetate | 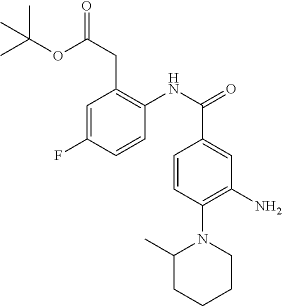 [SMILES: CC1CCCCN1c1ccc(C(=O)Nc2ccc(F)cc2CC(=O)OC(C)(C)C)cc1N] | LCMS-B; MS (ESIpos): m/z = 442.3 [M + H]⁺. |
| 20u | tert-butyl 2-(2-(3- amino-4-(2,6- dimethylpiperidin-1- yl)benzamido)-5- fluorophenyl)acetate | 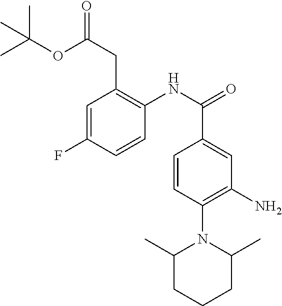 [SMILES: CC1CCCC(C)N1c1ccc(C(=O)Nc2ccc(F)cc2CC(=O)OC(C)(C)C)cc1N] | LCMS-B; MS (ESIpos): m/z = 456.3 [M + H]⁺. |
| 20v | tert-butyl 2-(2-(3- amino-4-(2- (hydroxymethyl)piperidin- 1-yl)benzamido)-5- fluorophenyl)acetate | 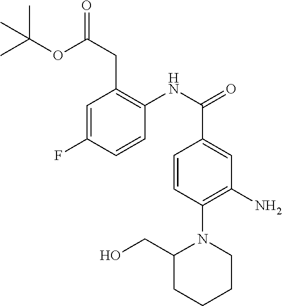 [SMILES: CC(C)(C)OC(=O)Cc1cc(F)ccc1NC(=O)c1ccc(N2CCCCC2CO)c(N)c1] | LCMS-B; MS (ESIpos): m/z = 458.3 [M + H]⁺. |
| 20w | tert-butyl 2-(2-(3- amino-4-(2- carbamoylpiperidin- 1-yl)benzamido)-5- fluorophenyl)acetate | 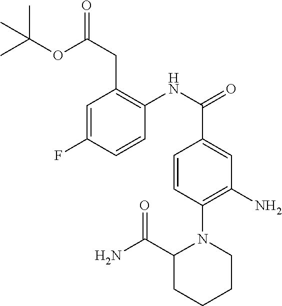 | LCMS-AO; MS (ESIpos): m/z = 454.2 [M + H]⁺. |
| 20x | tert-butyl 2-(2-(3- amino-4-(2- ethylpiperidin-1- yl)benzamido)-5- fluorophenyl)acetate | 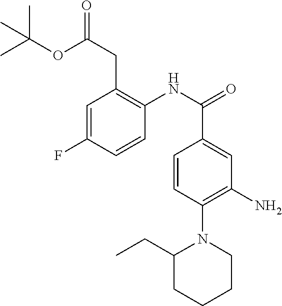 [SMILES: CCC1CCCCN1c1ccc(C(=O)Nc2ccc(F)cc2CC(=O)OC(C)(C)C)cc1N] | LCMS-A; MS (ESIpos): m/z = 456.3 [M + H]⁺. |
| 20y | tert-butyl 2-(2-(3- amino-4-(3- methylpiperidin-1- yl)benzamido)-5- fluorophenyl)acetate | 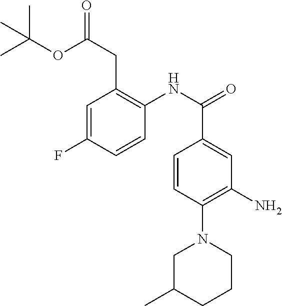 [SMILES: CC1CCCN(c2ccc(C(=O)Nc3ccc(F)cc3CC(=O)OC(C)(C)C)cc2N)C1] | LCMS-A; MS (ESIpos): m/z = 442.2 [M + H]⁺. |
| 20z | tert-butyl 2-(2-(3- amino-4-(3,5- dimethylpiperidin-1- yl)benzamido)-5- fluorophenyl)acetate | 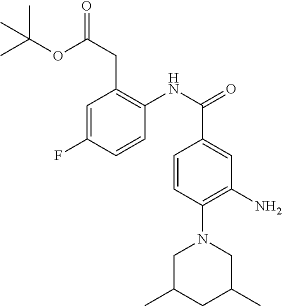 [SMILES: CC1CC(C)CN(c2ccc(C(=O)Nc3ccc(F)cc3CC(=O)OC(C)(C)C)cc2N)C1] | |
| 20aa | tert-butyl 2-(2-(3- amino-4-(4- hydroxypiperidin-1- yl)benzamido)-5- fluorophenyl)acetate | 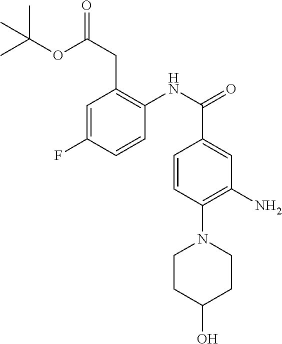 [SMILES: CC(C)(C)OC(=O)Cc1cc(F)ccc1NC(=O)c1ccc(N2CCC(O)CC2)c(N)c1] | LCMS-B; MS (ESIpos): m/z = 444.3 [M + H]⁺. |
| 20ab | tert-butyl (R)-2-(2- (3-amino-4-(2- methylpiperidin-1- yl)benzamido)-5- fluorophenyl)acetate | 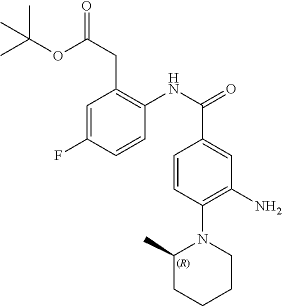 | LCMS-B; MS (ESIpos): m/z = 442.3 [M + H]⁺. |
| 20ac | tert-butyl (S)-2-(2- (3-amino-4-(2- methylpiperidin-1- yl)benzamido)-5- fluorophenyl)acetate | 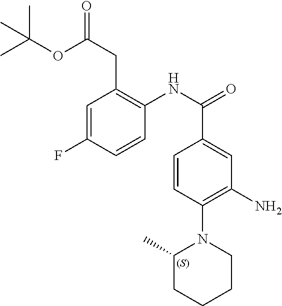 | LCMS-B; MS (ESIpos): m/z = 442.3 [M + H]⁺. |
| 20ad | tert-butyl (R)-2-(2- (3-amino-4-(3- methylpiperidin-1- yl)benzamido)-5- fluorophenyl)acetate | 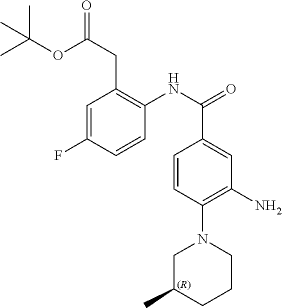 | LCMS-B; MS (ESIpos): m/z = 442.3 [M + H]⁺. |
| 20ae | tert-butyl (S)-2-(2- (3-amino-4-(3- methylpiperidin-1- yl)benzamido)-5- fluorophenyl)acetate | 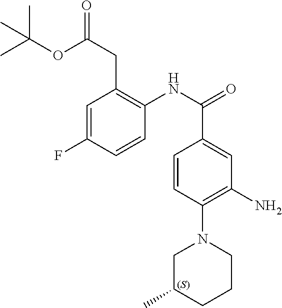 | LCMS-AH; MS (ESIpos): m/z = 442.2 [M + H]⁺. |
| 20af | tert-butyl 2-(2-(3- amino-4-(3- (difluoromethyl)piperidin-1- yl)benzamido)-5- fluorophenyl)acetate | 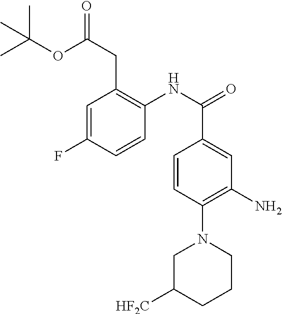 | LCMS-B; MS (ESIpos): m/z = 478.3 [M + H]⁺. |
| 20ag | tert-butyl 2-(2-(3- amino-4-(4- (difluoromethyl)piperidin-1- yl)benzamido)-5- fluorophenyl)acetate | 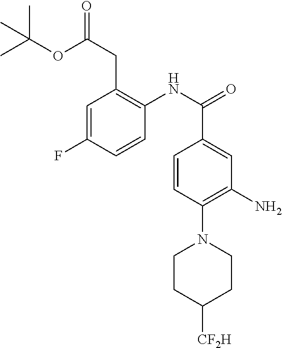 [SMILES: CC(C)(C)OC(=O)Cc1cc(F)ccc1NC(=O)c1ccc(N2CCC(C(F)F)CC2)c(N)c1] | LCMS-B; MS (ESIpos): m/z = 478.3 [M + H]⁺. |
| 20ah | tert-butyl 2-(2-(3- amino-4-(3- methoxypiperidin-1- yl)benzamido)-5- fluorophenyl)acetate | 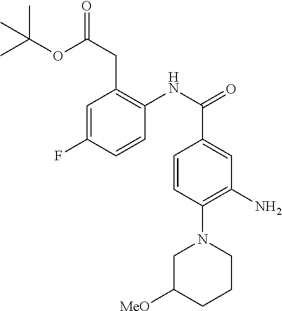 [SMILES: COC1CCCN(c2ccc(C(=O)Nc3ccc(F)cc3CC(=O)OC(C)(C)C)cc2N)C1] | LCMS-B; MS (ESIpos): m/z = 458.3 [M + H]⁺. |
| 20ai | tert-butyl 2-(2-(3- amino-4-(3- hydroxypiperidin-1- yl)benzamido)-5- fluorophenyl)acetate | 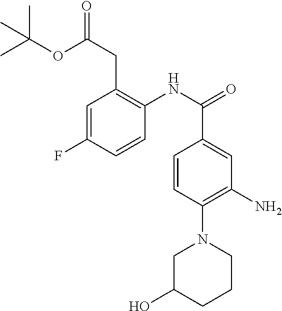 [SMILES: CC(C)(C)OC(=O)Cc1cc(F)ccc1NC(=O)c1ccc(N2CCCC(O)C2)c(N)c1] | LCMS-B; MS (ESIpos): m/z = 444.3 [M + H]⁺. |
| 20aj | tert-butyl 2-(2-(3- amino-4-(3- (hydroxymethyl)piperidin- 1-yl)benzamido)-5- fluorophenyl)acetate | 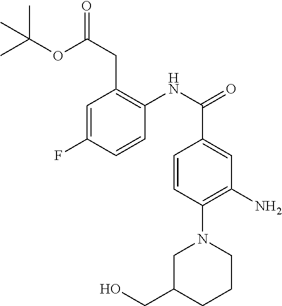 [SMILES: CC(C)(C)OC(=O)Cc1cc(F)ccc1NC(=O)c1ccc(N2CCCC(CO)C2)c(N)c1] | LCMS-AH; MS (ESIpos): m/z = 458.2 [M + H]⁺. |
| 20ak | tert-butyl 2-(2-(3- amino-4-(3- carbamoylpiperidin-1- yl)benzamido)-5- fluorophenyl)acetate | 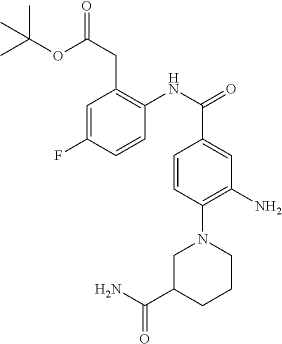 [SMILES: CC(C)(C)OC(=O)Cc1cc(F)ccc1NC(=O)c1ccc(N2CCCC(C(N)=O)C2)c(N)c1] | LCMS-B; MS (ESIpos): m/z = 471.3 [M + H]⁺. |
| 20al | tert-butyl 2-(2-(3- amino-4-(4- ethylpiperidin-1- yl)benzamido)-5- fluorophenyl)acetate | 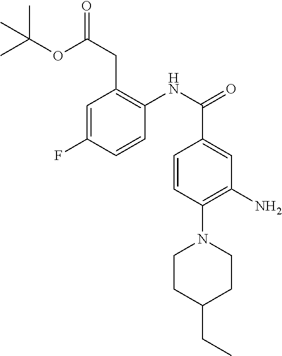 [SMILES: CCC1CCN(c2ccc(C(=O)Nc3ccc(F)cc3CC(=O)OC(C)(C)C)cc2N)CC1] | |
| 20an | tert-butyl (S)-2-(2- (3-amino-4-(2- ethylpiperidin-1- yl)benzamido)-5- fluorophenyl)acetate | 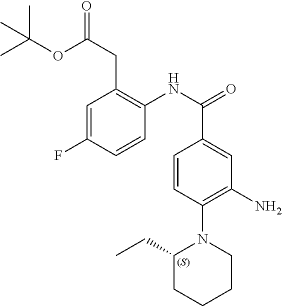 | LCMS-B; MS (ESIpos): m/z = 456.3 [M + H]⁺. |

### Intermediate 20f: tert-butyl 2-(2-(3-amino-4-(3-azabicyclo[3.1.0]hexan-3-yl)benzamido)-5-fluorophenyl)acetate

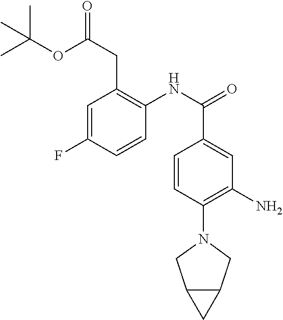 [SMILES: CC(C)(C)OC(=O)Cc1cc(F)ccc1NC(=O)c1ccc(N2CC3CC3C2)c(N)c1]

A mixture of intermediate 19f (340 mg, 746.47 μmol, 1 eq), iron (208.45 mg, 3.73 mmol, 5 eq) and NH₄Cl (399.29 mg, 7.46 mmol, 10 eq) in EtOH (10 mL) and H₂O (2 mL) was stirred at 80° C. for 1 hr under N₂ atmosphere. The reaction mixture was filtered and concentrated under reduced pressure to give a residue. The residue was partitioned between brine (30 mL) and ethyl acetate 30 (mL). The organic layer was washed with brine 60 mL, dried over Na₂SO₄, filtered, and concentrated under reduced pressure. The residue was purified by flash silica gel chromatography (Eluent of 0˜20% Ethyl acetate/Petroleum ether) to give the title compound (250 mg, 564.05 μmol, 75.56% yield, 96% purity) as a pale-yellow solid.

LCMS-S; MS (ESIpos): m/z=426.0 [M+H]⁺.

The following intermediates 20g-h were prepared in analogy to the intermediate 20f:

| Int. | Name | Structure | Analytical Data |
|---|---|---|---|
| 20g | tert-butyl 2-(2-(3- amino-4-(5- azaspiro[2.4] heptan-5- yl)benzamido)-5- fluorophenyl) acetate | 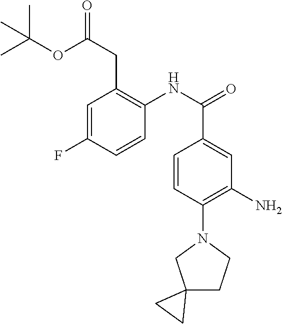 [SMILES: CC(C)(C)OC(=O)Cc1cc(F)ccc1NC(=O)c1ccc(N2CCC3(CC3)C2)c(N)c1] | ¹H NMR (400 MHz, CDCl₃) δ = 9.45 (br s, 1H), 7.93 (dd, J = 5.3, 9.0 Hz, 1H), 7.45-7.34 (m, 2H), 7.17-6.98 (m, 2H), 6.96 (dd, J = 2.9, 8.8 Hz, 1H), 3.62-3.11 (m, 6H), 2.13-1.93 (m, 2H), 1.47 (s, 9H), 0.82-0.59 (m, 4H). LCMS-B; MS (ESIpos): m/z = 440.3 [M + H]⁺. |
| 20h | tert-butyl 2-(2-(3- amino-4-((1s,4s)- 7- azabicyclo[2.2.1] heptan-7- yl)benzamido)-5- fluorophenyl) acetate | 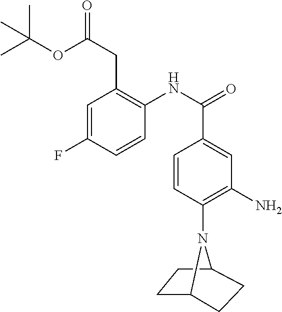 [SMILES: CC(C)(C)OC(=O)Cc1cc(F)ccc1NC(=O)c1ccc(N2C3CCC2CC3)c(N)c1] | LCMS-S; MS (ESIpos): m/z = 440.1 [M + H]⁺. |
| 20am | tert-butyl 2-(2-(3- amino-4-(6- azaspiro[2.5] octan-6- yl)benzamido)-5- fluorophenyl) acetate | 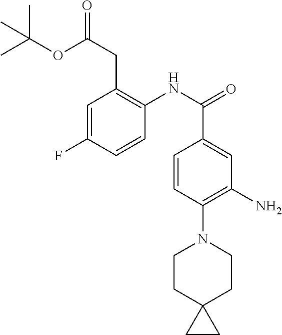 [SMILES: CC(C)(C)OC(=O)Cc1cc(F)ccc1NC(=O)c1ccc(N2CCC3(CC2)CC3)c(N)c1] | LCMS-B; MS (ESIpos): m/z = 454.2 [M + H]⁺. |

### Intermediate 21a: tert-butyl 2-(2-(4-(azetidin-1-yl)-3-(1-(2,2,2-trifluoroethyl)-1H-indazole-3-carboxamido)benzamido)-5-fluorophenyl)acetate

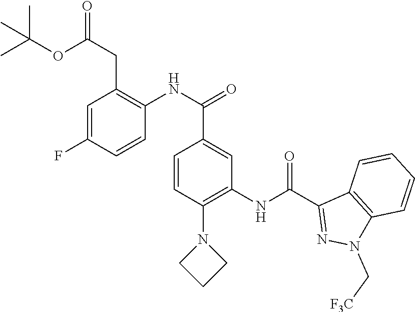 [SMILES: CC(C)(C)OC(=O)Cc1cc(F)ccc1NC(=O)c1ccc(N2CCC2)c(NC(=O)c2nn(CC(F)(F)F)c3ccccc23)c1]

A mixture of intermediate 20a (180 mg, 450.61 μmol, 1 eq) (crude), 1-(2,2,2-trifluoroethyl)indazole-3-carboxylic acid (132.03 mg, 540.73 μmol, 1.2 eq), HATU (257.00 mg, 675.92 μmol, 1.5 eq) and DIPEA (116.47 mg, 901.22 μmol, 2 eq) in DCM (2 mL) was stirred at 25° C. for 15 h. LCMS-S showed one main peak with desired mass was detected. The reaction mixture was concentrated under reduced pressure. The residue was purified by flash silica gel chromatography (Eluent of 0˜20% Ethyl acetate/Petroleum ether) to afford the title compound (260 mg, 415.59 μmol, 92.23% yield, 100% purity) as a pale-yellow solid.

LCMS-B; MS (ESIpos): m/z=626.3 [M+H]˜.

The following intermediates 21b-al were prepared in analogy to the intermediate 21a.

| Int. | Name | Structure | Analytical Data |
|---|---|---|---|
| 21b | tert-butyl 2-(2-(4- (4,4- difluoropiperidin-1- yl)-3-(1-(2,2,2- trifluoroethyl)-1H- indazole-3- carboxamido) benzamido)-5- fluorophenyl)acetate | 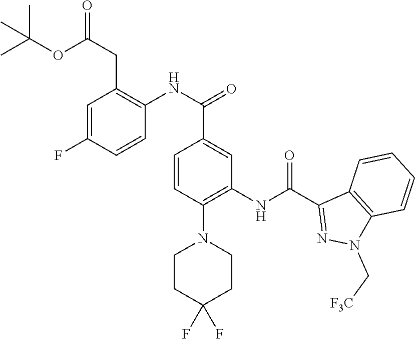 [SMILES: CC(C)(C)OC(=O)Cc1cc(F)ccc1NC(=O)c1ccc(N2CCC(F)(F)CC2)c(NC(=O)c2nn(CC(F)(F)F)c3ccccc23)c1] | LCMS-S; MS (ESIpos): m/z = 690.3 [M + H]⁺. |
| 21c | tert-butyl 2-(2-(4-(4- cyanopiperidin-1- yl)-3-(1-(2,2,2- trifluoroethyl)-1H- indazole-3- carboxamido) benzamido)-5- fluorophenyl)acetate | 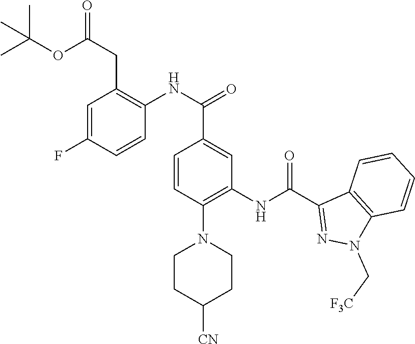 [SMILES: CC(C)(C)OC(=O)Cc1cc(F)ccc1NC(=O)c1ccc(N2CCC(C#N)CC2)c(NC(=O)c2nn(CC(F)(F)F)c3ccccc23)c1] | LCMS-S; MS (ESIpos): m/z = 679.3 [M + H]⁺. |
| 21d | tert-butyl 2-(2-(4-(2- azaspiro[3.3]heptan- 2-yl)-3-(1-(2,2,2- trifluoroethyl)-1H- indazole-3- carboxamido) benzamido)-5- fluorophenyl)acetate | 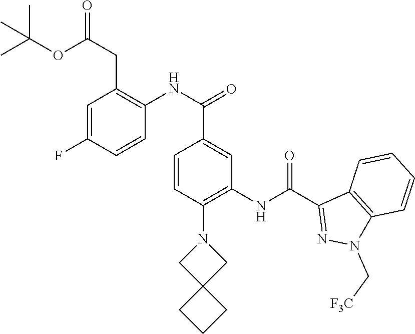 [SMILES: CC(C)(C)OC(=O)Cc1cc(F)ccc1NC(=O)c1ccc(N2CC3(CCC3)C2)c(NC(=O)c2nn(CC(F)(F)F)c3ccccc23)c1] | LCMS-AB; MS (ESIpos): m/z = 666.2 [M + H]⁺. |
| 21e | tert-butyl 2-(5- fluoro-2-(4- (pyrrolidin-1-yl)-3- (1-(2,2,2- trifluoroethyl)-1H- indazole-3- carboxamido) benzamido)phenyl) acetate | 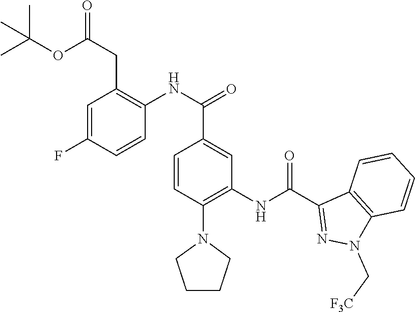 [SMILES: CC(C)(C)OC(=O)Cc1cc(F)ccc1NC(=O)c1ccc(N2CCCC2)c(NC(=O)c2nn(CC(F)(F)F)c3ccccc23)c1] | LCMS-A; MS (ESIpos): m/z = 640.2 [M + H]⁺. |
| 21f | tert-butyl 2-(2-(4-(3- azabicyclo[3.1.0] hexan-3-yl)-3-(1-(2,2,2- trifluoroethyl)-1H- indazole-3- carboxamido) benzamido)-5- fluorophenyl)acetate | 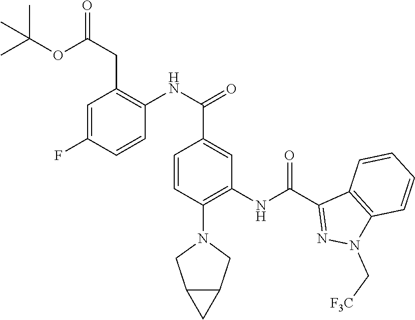 [SMILES: CC(C)(C)OC(=O)Cc1cc(F)ccc1NC(=O)c1ccc(N2CC3CC3C2)c(NC(=O)c2nn(CC(F)(F)F)c3ccccc23)c1] | LCMS-B; MS (ESIpos): m/z = 652.3 [M + H]⁺. |
| 21g | tert-butyl 2-(2-(4-(5- azaspiro[2.4]heptan- 5-yl)-3-(1-(2,2,2- trifluoroethyl)-1H- indazole-3- carboxamido) benzamido)-5- fluorophenyl)acetate | 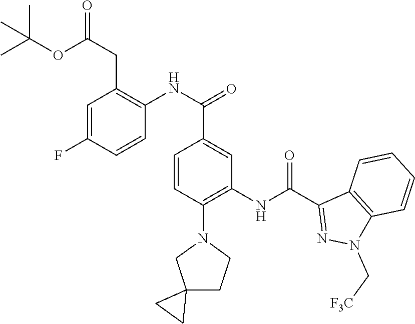 [SMILES: CC(C)(C)OC(=O)Cc1cc(F)ccc1NC(=O)c1ccc(N2CCC3(CC3)C2)c(NC(=O)c2nn(CC(F)(F)F)c3ccccc23)c1] | LCMS-B; MS (ESIpos): m/z = 666.3 [M + H]⁺. |
| 21h | tert-butyl 2-(2-(4- ((1s,4s)-7- azabicyclo[2.2.1] heptan-7-yl)-3-(1-(2,2,2- trifluoroethyl)-1H- indazole-3- carboxamido) benzamido)-5- fluorophenyl)acetate | 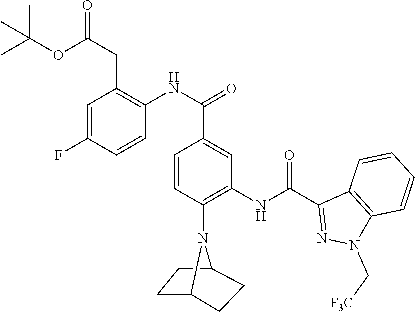 [SMILES: CC(C)(C)OC(=O)Cc1cc(F)ccc1NC(=O)c1ccc(N2C3CCC2CC3)c(NC(=O)c2nn(CC(F)(F)F)c3ccccc23)c1] | LCMS-B; MS (ESIpos): m/z = 666.3 [M + H]⁺. |
| 21i | tert-butyl 2-(5- fluoro-2-(4- morpholino-3-(1- (2,2,2- trifluoroethyl)-1H- indazole-3- carboxamido)benzamido) phenyl)acetate | 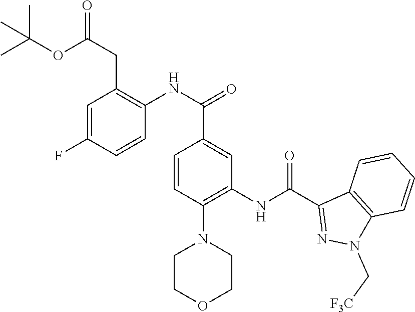 [SMILES: CC(C)(C)OC(=O)Cc1cc(F)ccc1NC(=O)c1ccc(N2CCOCC2)c(NC(=O)c2nn(CC(F)(F)F)c3ccccc23)c1] | LCMS-AH; MS (ESIpos): m/z = 656.3 [M + H]⁺. |
| 21j | tert-butyl 2-(5- fluoro-2-(4-(4- methylpiperazin-1- yl)-3-(1-(2,2,2- trifluoroethyl)-1H- indazole-3- carboxamido)benzamido) phenyl)acetate | 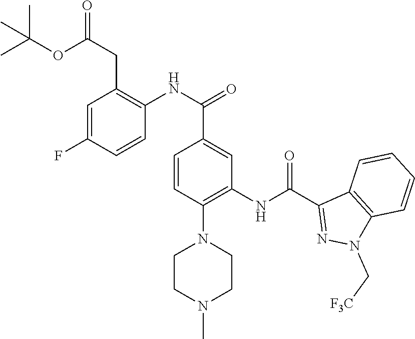 [SMILES: CN1CCN(c2ccc(C(=O)Nc3ccc(F)cc3CC(=O)OC(C)(C)C)cc2NC(=O)c2nn(CC(F)(F)F)c3ccccc23)CC1] | LCMS-Y; MS (ESIpos): m/z = 669.4 [M + H]⁺. |
| 21k | tert-butyl 2-(2-(4-(2- azabicyclo[2.2.1] heptan-2-yl)-3-(1-(2,2,2- trifluoroethyl)-1H- indazole-3- carboxamido) benzamido)-5- fluorophenyl)acetate | 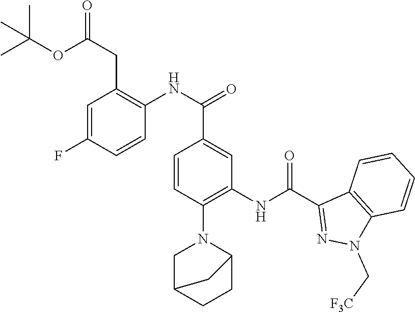 [SMILES: CC(C)(C)OC(=O)Cc1cc(F)ccc1NC(=O)c1ccc(N2CC3CCC2C3)c(NC(=O)c2nn(CC(F)(F)F)c3ccccc23)c1] | LCMS-A; MS (ESIpos): m/z = 666.2 [M + H]⁺. |
| 21l | tert-butyl 2-(5- fluoro-2-(4-(2- oxopiperidin-1-yl)- 3-(1-(2,2,2- trifluoroethyl)-1H- indazole-3- carboxamido)benzamido) phenyl)acetate | 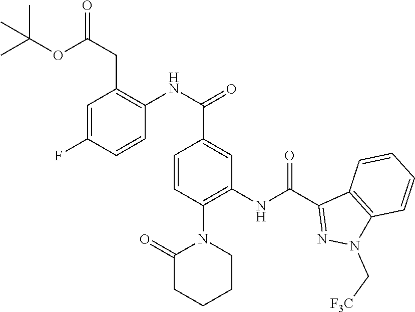 [SMILES: CC(C)(C)OC(=O)Cc1cc(F)ccc1NC(=O)c1ccc(N2CCCCC2=O)c(NC(=O)c2nn(CC(F)(F)F)c3ccccc23)c1] | LCMS-S; MS (ESIpos): m/z = 668.2 [M + H]⁺. |
| 21m | tert-butyl 2-(5- fluoro-2-(4-(2- oxooxazolidin-3-yl)- 3-(1-(2,2,2- trifluoroethyl)-1H- indazole-3- carboxamido)benzamido) phenyl)acetate | 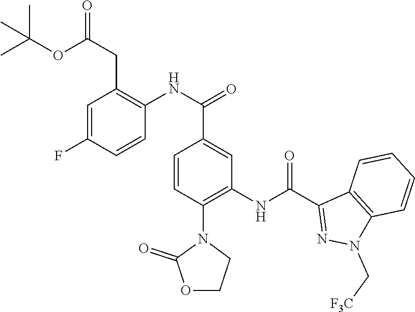 [SMILES: CC(C)(C)OC(=O)Cc1cc(F)ccc1NC(=O)c1ccc(N2CCOC2=O)c(NC(=O)c2nn(CC(F)(F)F)c3ccccc23)c1] | LCMS-B; MS (ESIpos): m/z = 600.2 [M − 55]⁺. |
| 21n | tert-butyl 2-(2-(4- (4,4- dimethylpiperidin-1- yl)-3-(1-(2,2,2- trifluoroethyl)-1H- indazole-3- carboxamido) benzamido)-5- fluorophenyl)acetate | 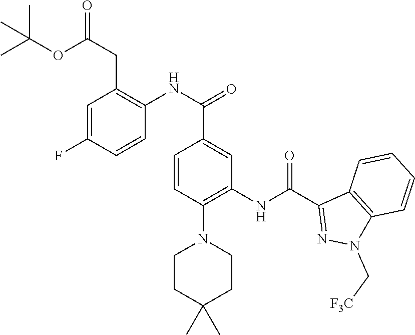 [SMILES: CC1(C)CCN(c2ccc(C(=O)Nc3ccc(F)cc3CC(=O)OC(C)(C)C)cc2NC(=O)c2nn(CC(F)(F)F)c3ccccc23)CC1] | LCMS-A; MS (ESIpos): m/z = 682.2 [M + H]⁺. |
| 21o | tert-butyl 2-(2-(4- (3,3- difluoropiperidin-1- yl)-3-(1-(2,2,2- trifluoroethyl)-1H- indazole-3- carboxamido) benzamido)-5- fluorophenyl)acetate | 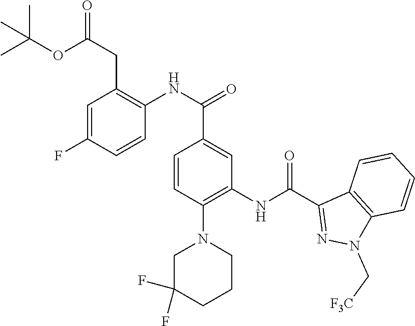 [SMILES: CC(C)(C)OC(=O)Cc1cc(F)ccc1NC(=O)c1ccc(N2CCCC(F)(F)C2)c(NC(=O)c2nn(CC(F)(F)F)c3ccccc23)c1] | LCMS-S; MS (ESIpos): m/z = 690.2 [M + H]⁺. |
| 21p | tert-butyl 2-(2-(4- (1,4-oxazepan-4-yl)- 3-(1-(2,2,2- trifluoroethyl)-1H- indazole-3- carboxamido) benzamido)-5- fluorophenyl)acetate | 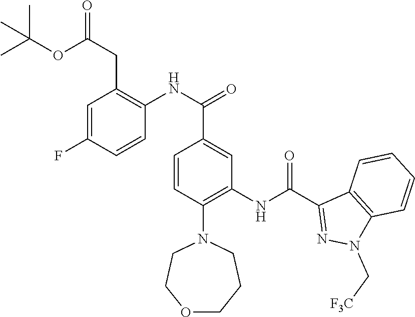 [SMILES: CC(C)(C)OC(=O)Cc1cc(F)ccc1NC(=O)c1ccc(N2CCCOCC2)c(NC(=O)c2nn(CC(F)(F)F)c3ccccc23)c1] | LCMS-B; MS (ESIpos): m/z = 670.3 [M + H]⁺. |
| 21q | tert-butyl 2-(5- fluoro-2-(4-(4- methylpiperidin-1- yl)-3-(1-(2,2,2- trifluoroethyl)-1H- indazole-3- carboxamido) benzamido)phenyl) acetate | 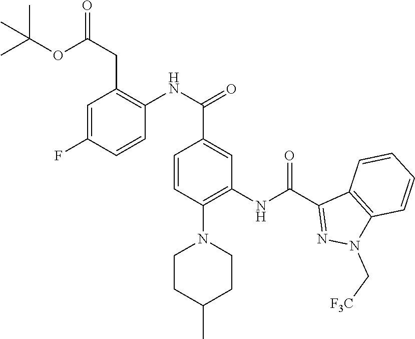 [SMILES: CC1CCN(c2ccc(C(=O)Nc3ccc(F)cc3CC(=O)OC(C)(C)C)cc2NC(=O)c2nn(CC(F)(F)F)c3ccccc23)CC1] | LCMS-B; MS (ESIpos): m/z = 668.3 [M + H]⁺. |
| 21r | tert-butyl 2-(5- fluoro-2-(4-(4- methoxypiperidin-1- yl)-3-(1-(2,2,2- trifluoroethyl)-1H- indazole-3- carboxamido) benzamido)phenyl) acetate | 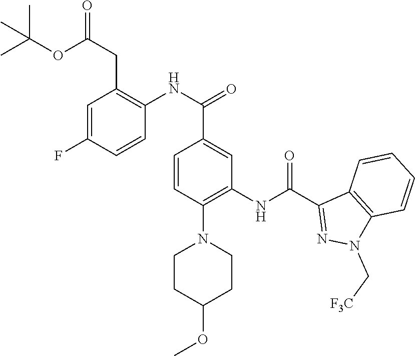 [SMILES: COC1CCN(c2ccc(C(=O)Nc3ccc(F)cc3CC(=O)OC(C)(C)C)cc2NC(=O)c2nn(CC(F)(F)F)c3ccccc23)CC1] | LCMS-A; MS (ESIpos): m/z = 684.2 [M + H]⁺. |
| 21s | tert-butyl 2-(2-(4- (1,1-dioxido- thiomorpholino)- 3-(1-(2,2,2- trifluoroethyl)-1H- indazole-3- carboxamido) benzamido)-5- fluorophenyl)acetate | 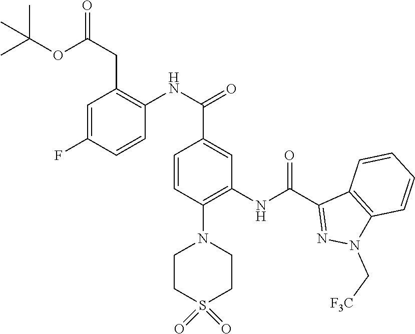 [SMILES: CC(C)(C)OC(=O)Cc1cc(F)ccc1NC(=O)c1ccc(N2CCS(=O)(=O)CC2)c(NC(=O)c2nn(CC(F)(F)F)c3ccccc23)c1] | LCMS-B; MS (ESIpos): m/z = 704.2 [M + H]⁺. |
| 21t | tert-butyl 2-(5- fluoro-2-(4-(2- methylpiperidin-1- yl)-3-(1-(2,2,2- trifluoroethyl)-1H- indazole-3- carboxamido) benzamido)phenyl) acetate | 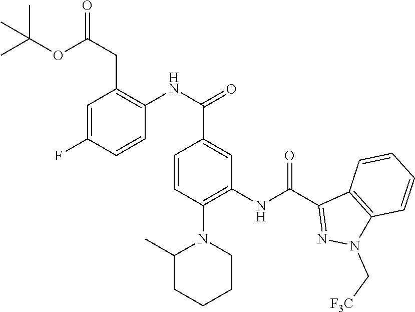 [SMILES: CC1CCCCN1c1ccc(C(=O)Nc2ccc(F)cc2CC(=O)OC(C)(C)C)cc1NC(=O)c1nn(CC(F)(F)F)c2ccccc12] | LCMS-B; MS (ESIpos): m/z = 668.3 [M + H]⁺. |
| 21u | tert-butyl 2-(2-(4- (2,6- dimethylpiperidin-1- yl)-3-(1-(2,2,2- trifluoroethyl)-1H- indazole-3- carboxamido) benzamido)-5- fluorophenyl)acetate | 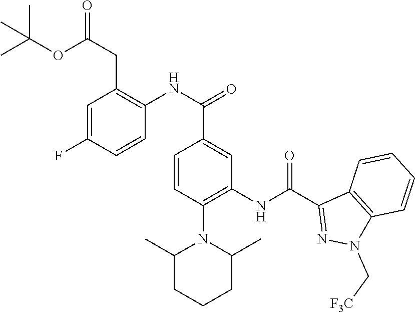 [SMILES: CC1CCCC(C)N1c1ccc(C(=O)Nc2ccc(F)cc2CC(=O)OC(C)(C)C)cc1NC(=O)c1nn(CC(F)(F)F)c2ccccc12] | ¹H NMR (400 MHz, CDCl₃) δ = 11.00 (s, 1H), 9.42 (s, 1H), 9.30 (d, J = 2.0 Hz, 1H), 8.50 (d, J = 7.9 Hz, 1H), 7.94 (dd, J = 5.3, 8.9 Hz, 1H), 7.82 (dd, J = 2.0, 8.1 Hz, 1H), 7.57-7.53 (m, 1H), 7.53- 7.49 (m, 1H), 7.43- 7.37 (m, 2H), 7.07 (dt, J = 2.7, 8.5 Hz, 1H), 7.01 (dd, J = 2.9, 8.9 Hz, 1H), 5.03 (q, J = 8.3 Hz, 2H), 3.69-3.64 (m, 2H), 2.96- 2.87 (m, 2H), 1.93- 1.83 (m, 3H), 1.58-1.50 (m, 3H), 1.48 (s, 9H), 0.78 (d, J = 6.3 Hz, 6H). LCMS-AO; MS |
| | | | (ESIpos): m/z = 682.4 |
| | | | [M + H]⁺. |
| 21v | tert-butyl 2-(5- fluoro-2-(4-(2- (hydroxymethyl) piperidin-1-yl)-3-(1- (2,2,2- trifluoroethyl)-1H- indazole-3- carboxamido) benzamido)phenyl) acetate | 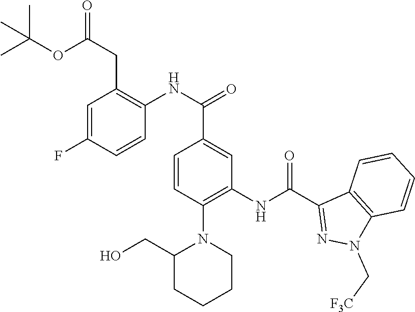 [SMILES: CC(C)(C)OC(=O)Cc1cc(F)ccc1NC(=O)c1ccc(N2CCCCC2CO)c(NC(=O)c2nn(CC(F)(F)F)c3ccccc23)c1] | ¹H NMR (400 MHz, CDCl₃) δ = 10.55 (s, 1H), 9.53 (s, 1H), 9.27 (d, J = 2.1 Hz, 1H), 8.48 (d, J = 8.3 Hz, 1H), 7.94 (dd, J = 5.4, 8.8 Hz, 1H), 7.81 (dd, J = 2.1, 8.3 Hz, 1H), 7.58-7.53 (m, 1H), 7.52- 7.48 (m, 1H), 7.43- 7.37 (m, 2H), 7.07 (dt, J = 2.9, 8.4 Hz, 1H), 7.00 (dd, J = 2.9, 8.9 Hz, 1H), 5.06-4.98 (m, 2H), 3.65 (s, 2H), 3.49 (s, 2H), 3.18- 3.12 (m, 1H), 3.04 (d, J = 11.8 Hz, 1H), 2.78- 2.71 (m, 1H), 2.05-2.01 (m, 1H), 1.98 (d, J = 13.4 Hz, 1H), 1.88-1.77 (m, |
| | | | 3H), 1.60-1.55 (m, 1H), |
| | | | 1.49 (s, 9H). |
| | | | LCMS-B; MS (ESIpos): |
| | | | m/z = 684.3 [M + H]⁺. |
| 21w | tert-butyl 2-(2-(4-(2- carbamoylpiperidin- 1-yl)-3-(1-(2,2,2- trifluoroethyl)-1H- indazole-3- carboxamido) benzamido)-5- fluorophenyl)acetate | 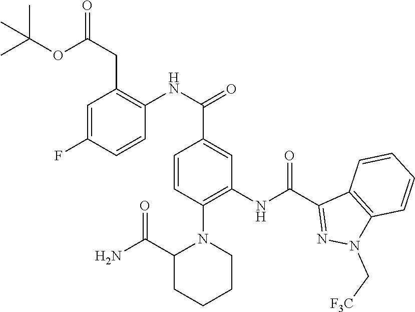 [SMILES: CC(C)(C)OC(=O)Cc1cc(F)ccc1NC(=O)c1ccc(N2CCCCC2C(N)=O)c(NC(=O)c2nn(CC(F)(F)F)c3ccccc23)c1] | LCMS-B; MS (ESIpos): m/z = 697.3 [M + H]⁺. |
| 21x | tert-butyl 2-(2-(4-(2- ethylpiperidin-1-yl)- 3-(1-(2,2,2- trifluoroethyl)-1H- indazole-3- carboxamido) benzamido)-5- fluorophenyl)acetate | 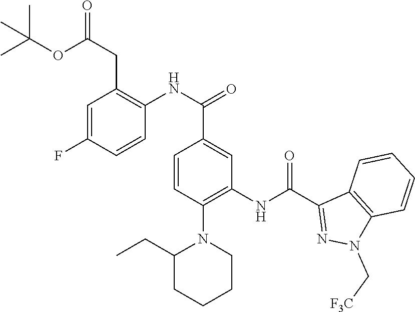 [SMILES: CCC1CCCCN1c1ccc(C(=O)Nc2ccc(F)cc2CC(=O)OC(C)(C)C)cc1NC(=O)c1nn(CC(F)(F)F)c2ccccc12] | LCMS-A; MS (ESIpos): m/z = 682.2 [M + H]⁺. |
| 21y | tert-butyl 2-(5- fluoro-2-(4-(3- methylpiperidin-1- yl)-3-(1-(2,2,2- trifluoroethyl)-1H- indazole-3- carboxamido) benzamido)phenyl) acetate | 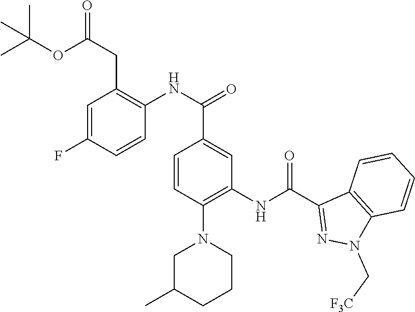 [SMILES: CC1CCCN(c2ccc(C(=O)Nc3ccc(F)cc3CC(=O)OC(C)(C)C)cc2NC(=O)c2nn(CC(F)(F)F)c3ccccc23)C1] | LCMS-A; MS (ESIpos): m/z = 668.2 [M + H]⁺. |
| 21z | tert-butyl 2-(2-(4- (3,5- dimethylpiperidin-1- yl)-3-(1-(2,2,2- trifluoroethyl)-1H- indazole-3- carboxamido) benzamido)-5- fluorophenyl)acetate | 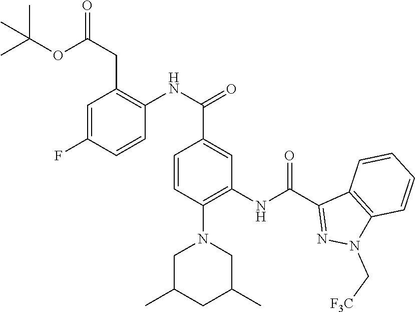 [SMILES: CC1CC(C)CN(c2ccc(C(=O)Nc3ccc(F)cc3CC(=O)OC(C)(C)C)cc2NC(=O)c2nn(CC(F)(F)F)c3ccccc23)C1] | LCMS-S; MS (ESIpos): m/z = 682.3 [M + H]⁺. |
| 21aa | tert-butyl 2-(5- fluoro-2-(4-(4- hydroxypiperidin-1- yl)-3-(1-(2,2,2- trifluoroethyl)-1H- indazole-3- carboxamido) benzamido)phenyl) acetate | 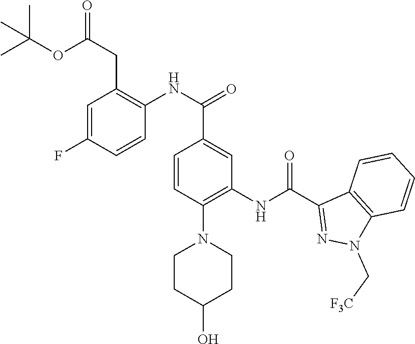 [SMILES: CC(C)(C)OC(=O)Cc1cc(F)ccc1NC(=O)c1ccc(N2CCC(O)CC2)c(NC(=O)c2nn(CC(F)(F)F)c3ccccc23)c1] | ¹H NMR (400 MHz, CDCl₃) δ = 9.97 (s, 1H), 9.49 (s, 1H), 9.20 (d, J = 1.8 Hz, 1H), 8.51 (d, J = 8.1 Hz, 1H), 7.94 (dd, J = 5.4, 8.9 Hz, 1H), 7.80 (dd, J = 1.8, 8.3 Hz, 1H), 7.55 (d, J = 6.9 Hz, 1H), 7.52-7.49 (m, 1H), 7.40 (t, J = 7.4 Hz, 1H), 7.29 (s, 1H), 7.10-7.03 (m, 1H), 7.00 (dd, J = 2.8, 8.9 Hz, 1H), 5.03 (q, J = 8.1 Hz, 2H), 4.05-3.98 (m, 1H), 3.64 (s, 2H), 3.25- 3.17 (m, 2H), 2.90-2.84 (m, 2H), 2.20-2.10 (m, 2H), 1.98-1.87 (m, 2H), 1.48 (s, 9H). LCMS-AO; MS (ESIpos): m/z = 670.3 |
| | | | [M + H]⁺ |
| 21ab | tert-butyl (R)-2-(5- fluoro-2-(4-(2- methylpiperidin-1- yl)-3-(1-(2,2,2- trifluoroethyl)-1H- indazole-3- carboxamido) benzamido)phenyl) acetate | 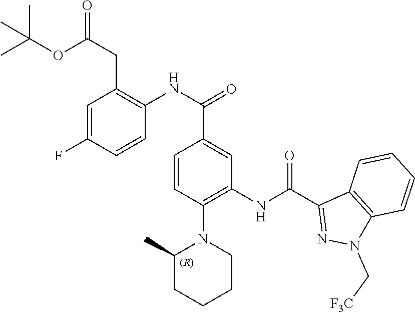 | LCMS-B; MS (ESIpos): m/z = 668.3 [M + H]⁺. |
| 21ac | tert-butyl (S)-2-(5- fluoro-2-(4-(2- methylpiperidin-1- yl)-3-(1-(2,2,2- trifluoroethyl)-1H- indazole-3- carboxamido) benzamido)phenyl) acetate | 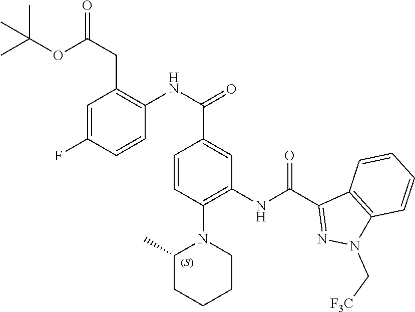 | ¹H NMR (400 MHz, DMSO-d₆) δ = 10.62 (s, 1H), 9.95 (s, 1H), 9.05 (d, J = 2.1 Hz, 1H), 8.31 (d, J = 8.2 Hz, 1H), 8.00 (d, J = 8.6 Hz, 1H), 7.74 (dd, J = 2.0, 8.3 Hz, 1H), 7.62 (t, J = 7.7 Hz, 1H), 7.52- 7.39 (m, 3H), 7.24-7.15 (m, 2H), 5.65 (q, J = 9.0 Hz, 2H), 3.68 (s, 2H), 3.15-3.05 (m, 1H), 2.89 (d, J = 11.1 Hz, 1H), 2.77- 2.68 (m, 1H), 1.93- 1.70 (m, 4H), 1.55-1.45 (m, 2H), 1.28 (s, 9H), 0.81 (d, J = 6.1 Hz, 3H). LCMS-AO; MS (ESIpos): m/z = 668.4 |
| | | | [M + H]⁺. |
| 21ad | tert-butyl (R)-2-(5- fluoro-2-(4-(3- methylpiperidin-1- yl)-3-(1-(2,2,2- trifluoroethyl)-1H- indazole-3- carboxamido) benzamido)phenyl) acetate | 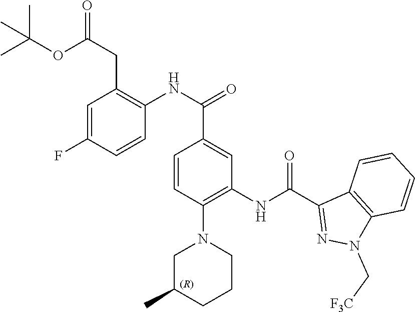 | LCMS-B; MS (ESIpos): m/z = 668.3 [M + H]⁺. |
| 21ae | tert-butyl (S)-2-(5- fluoro-2-(4-(3- methylpiperidin-1- yl)-3-(1-(2,2,2- trifluoroethyl)-1H- indazole-3- carboxamido) benzamido)phenyl) acetate | 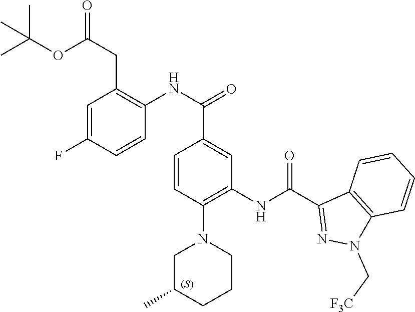 | LCMS-Y; MS (ESIpos): m/z = 668.2 [M + H]⁺. |
| 21af | tert-butyl 2-(2-(4-(3- (difluoromethyl) piperidin-1-yl)-3-(1- (2,2,2- trifluoroethyl)-1H- indazole-3- carboxamido) benzamido)-5- fluorophenyl)acetate | 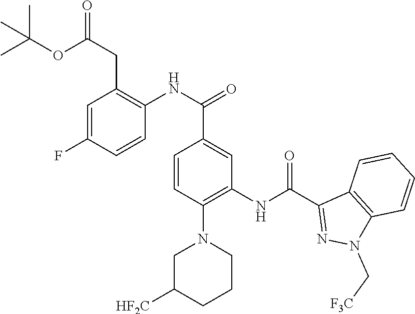 | ¹H NMR (400 MHz, DMSO-d₆) δ = 9.98 (s, 1H), 9.94 (s, 1H), 8.99 (d, J = 2.0 Hz, 1H), 8.31 (d, J = 8.1 Hz, 1H), 7.99 (d, J = 8.6 Hz, 1H), 7.75 (dd, J = 2.0, 8.3 Hz, 1H), 7.63 (t, J = 7.7 Hz, 1H), 7.47- 7.39 (m, 3H), 7.23-7.17 (m, 2H), 6.10 (dt, J = 5.0, 56.5 Hz, 1H), 5.66 (q, J = 9.0 Hz, 2H), 3.67 (s, 2H), 3.15 (d, J = 9.8 Hz, 1H), 3.02 (d, J = 11.2 Hz, 1H), 2.80-2.72 (m, 2H), 1.95- 1.76 (m, 4H), 1.49- 1.40 (m, 1H), 1.30 (s, 9H). LCMS-AO; MS |
| | | | (ESIpos): m/z = 704.4 |
| | | | [M + H]⁺. |
| 21ag | tert-butyl 2-(2-(4-(4- (difluoromethyl) piperidin-1-yl)-3-(1- (2,2,2- trifluoroethyl)-1H- indazole-3- carboxamido) benzamido)-5- fluorophenyl)acetate | 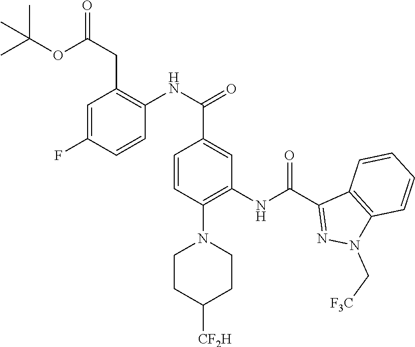 [SMILES: CC(C)(C)OC(=O)Cc1cc(F)ccc1NC(=O)c1ccc(N2CCC(C(F)F)CC2)c(NC(=O)c2nn(CC(F)(F)F)c3ccccc23)c1] | ¹H NMR (400 MHz, DMSO-d₆) δ = 10.07 (s, 1H), 9.94 (s, 1H), 9.00 (d, J = 1.9 Hz, 1H), 8.33 (d, J = 8.1 Hz, 1H), 7.99 (d, J = 8.6 Hz, 1H), 7.75 (dd, J = 1.9, 8.3 Hz, 1H), 7.68-7.59 (m, 1H), 7.48- 7.43 (m, 1H), 7.41-7.39 (m, 1H), 7.24-7.13 (m, 3H), 6.21-5.91 (m, 1H), 5.63 (q, J = 9.0 Hz, 2H), 3.68 (s, 2H), 3.15 (d, J = 11.4 Hz, 2H), 2.88-2.79 (m, 2H), 2.60-2.55 (m, 1H), 1.91-1.79 (m, 4H), 1.31 (s, 9H). LCMS-B; MS (ESIpos): m/z = 704.3 [M + H]⁺. |
| 21ah | tert-butyl 2-(5- fluoro-2-(4-(3- methoxypiperidin-1- yl)-3-(1-(2,2,2- trifluoroethyl)-1H- indazole-3- carboxamido) benzamido)phenyl) acetate | 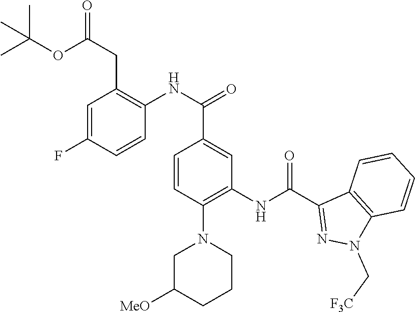 [SMILES: COC1CCCN(c2ccc(C(=O)Nc3ccc(F)cc3CC(=O)OC(C)(C)C)cc2NC(=O)c2nn(CC(F)(F)F)c3ccccc23)C1] | LCMS-AO; MS (ESIpos): m/z = 684.4 [M + H]⁺. |
| 21ai | tert-butyl 2-(5- fluoro-2-(4-(3- hydroxypiperidin-1- yl)-3-(1-(2,2,2- trifluoroethyl)-1H- indazole-3- carboxamido) benzamido)phenyl) acetate | 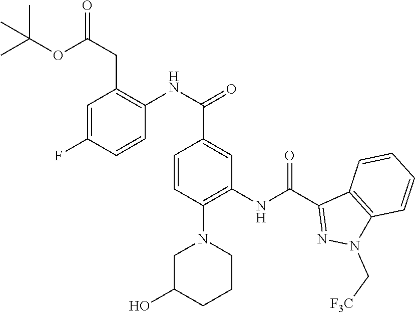 [SMILES: CC(C)(C)OC(=O)Cc1cc(F)ccc1NC(=O)c1ccc(N2CCCC(O)C2)c(NC(=O)c2nn(CC(F)(F)F)c3ccccc23)c1] | LCMS-AO; MS (ESIpos): m/z = 670.3 [M + H]⁺. |
| 21aj | tert-butyl 2-(5- fluoro-2-(4-(3- (hydroxymethyl) piperidin-1-yl)-3-(1- (2,2,2- trifluoroethyl)-1H- indazole-3- carboxamido) benzamido)phenyl) acetate | 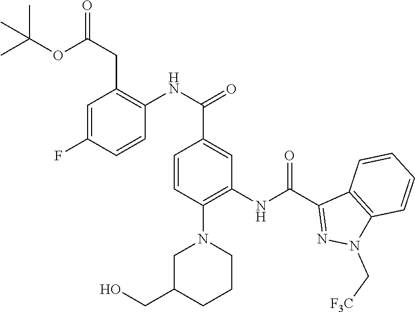 [SMILES: CC(C)(C)OC(=O)Cc1cc(F)ccc1NC(=O)c1ccc(N2CCCC(CO)C2)c(NC(=O)c2nn(CC(F)(F)F)c3ccccc23)c1] | LCMS-Y; MS (ESIpos): m/z = 684.2 [M + H]⁺. |
| 21ak | tert-butyl 2-(2-(4-(3- carbamoylpiperidin- 1-yl)-3-(1-(2,2,2- trifluoroethyl)-1H- indazole-3- carboxamido) benzamido)-5- fluorophenyl)acetate | 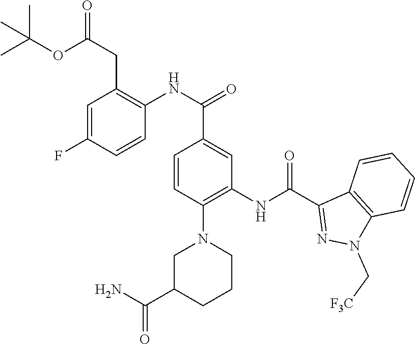 [SMILES: CC(C)(C)OC(=O)Cc1cc(F)ccc1NC(=O)c1ccc(N2CCCC(C(N)=O)C2)c(NC(=O)c2nn(CC(F)(F)F)c3ccccc23)c1] | LCMS-B; MS (ESIpos): m/z = 697.3 [M + H]⁺. |
| 21al | tert-butyl 2-(2-(4-(4- ethylpiperidin-1-yl)- 3-(1-(2,2,2- trifluoroethyl)-1H- indazole-3- carboxamido) benzamido)-5- fluorophenyl)acetate | 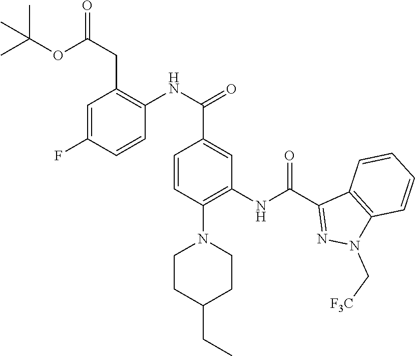 [SMILES: CCC1CCN(c2ccc(C(=O)Nc3ccc(F)cc3CC(=O)OC(C)(C)C)cc2NC(=O)c2nn(CC(F)(F)F)c3ccccc23)CC1] | LCMS-B; MS (ESIpos): m/z = 682.3 [M + H]⁺. |
| 21am | tert-butyl 2-(2-(4-(6- azaspiro[2.5]octan- 6-yl)-3-(1-(2,2,2- trifluoroethyl)-1H- indazole-3- carboxamido) benzamido)-5- fluorophenyl)acetate | 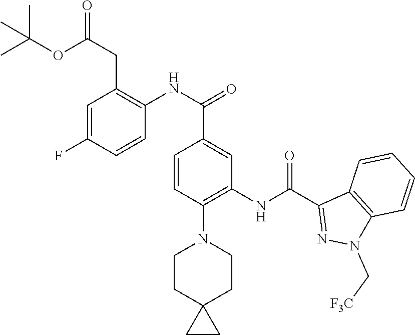 [SMILES: CC(C)(C)OC(=O)Cc1cc(F)ccc1NC(=O)c1ccc(N2CCC3(CC2)CC3)c(NC(=O)c2nn(CC(F)(F)F)c3ccccc23)c1] | LCMS-B; MS (ESIpos): m/z = 680.3 [M + H]⁺. |
| 20an | tert-butyl (S)-2-(2- (4-(2-ethylpiperidin- 1-yl)-3-(1-(2,2,2- trifluoroethyl)-1H- indazole-3- carboxamido) benzamido)-5- fluorophenyl)acetate | 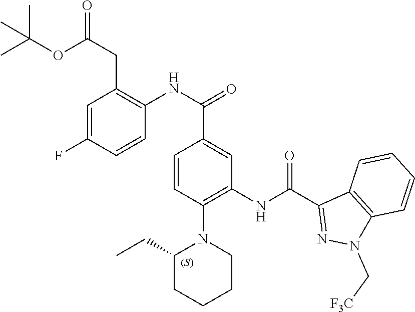 | LCMS-B; MS (ESIpos): m/z = 682.3 [M + H]⁺. |

### Intermediate 22: 4-piperidin-1-yl)-3-(l-(2,2,2-trifluoroethyl)-1H-indazole-3-carboxamido)benzoic acid

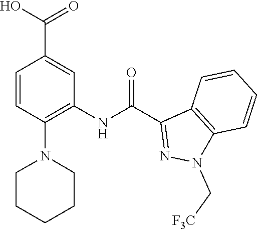 [SMILES: O=C(O)c1ccc(N2CCCCC2)c(NC(=O)c2nn(CC(F)(F)F)c3ccccc23)c1]

3-amino-4-(piperidin-1-yl)benzoic acid (1.82 g, 1.0 Eq, 8.25 mmol) was dissolved in THF (30 mL) and a saturated solution of NaHCO₃ (30 mL), while vigorously stirring the solution was cooled with an ice bath and a solution of 1-(2,2,2-trifluoroethyl)-1H-indazole-3-carbonyl chloride (2.17 g, 1 Eq, 8.25 mmol) in THF (20 mL) was added dropwise to it over few minutes, the reaction was stirred for two hours at room temperature. After cooling the reaction with an ice bath, the reaction's pH was adjusted to acidic by addition of HCl 1N causing precipitation of the product, further 300 mL of water were added. The precipitate was collected by vacuum filtration, was washed with water, and triturated with hexane (3×). The solid was then dried at high vacuum and directly used in the next step.

¹H NMR (400 MHz, DMSO-d₆) δ 12.84 (br s, 1H), 9.91 (s, 1H), 9.02 (d, J=1.8 Hz, 1H), 8.32 (d, J=8.1 Hz, 1H), 8.00 (d, J=8.6 Hz, 1H), 7.71 (dd, J=8.1, 2.0 Hz, 1H), 7.62 (ddd, J=8.4, 7.0, 1.0 Hz, 1H), 7.44 (t, J=7.6 Hz, 1H), 7.33 (d, J=8.4 Hz, 1H), 5.66 (q, J=9.0 Hz, 2H), 2.92-2.85 (m, 4H), 1.84-1.76 (m, 4H), 1.60 (br d, J=3.5 Hz, 2H).

LC-MS: Rₜ=1.45 min. MS (ESI+): m/z=447.3 [M+H]⁺.

### Intermediate 23: 4-(Piperidin-1-yl)-3-(1-(2,2,2-trifluoroethyl)-1H-indazole-3-carboxamido)benzoyl chloride

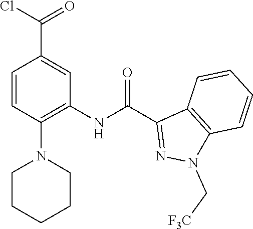 [SMILES: O=C(Cl)c1ccc(N2CCCCC2)c(NC(=O)c2nn(CC(F)(F)F)c3ccccc23)c1]

4-(Piperidin-1-yl)-3-(1-(2,2,2-trifluoroethyl)-1H-indazole-3-carboxamido)benzoic acid (160 mg, 86% Wt, 1 Eq, 308 μmol) was suspended in DCM (3 mL) under an inert atmosphere, oxalyl chloride (58.7 mg, 40.5 μL, 1.5 Eq, 462 μmol) followed by catalytic DMF were added at 0° C. The reaction was stirred for 4 hours letting the ice bath melting by itself. The solvent was evaporated in vacuo and the residue thus obtained redissolved in DCM and evaporated twice. The crude acyl chloride was directly used in the next step.

### Example 1: 2-(5-fluoro-2-(4-(piperidin-1-yl)-3-(1-(2,2,2-trifluoroethyl)-1H-indazole-3-carboxamido)benzamido)phenyl)acetic acid

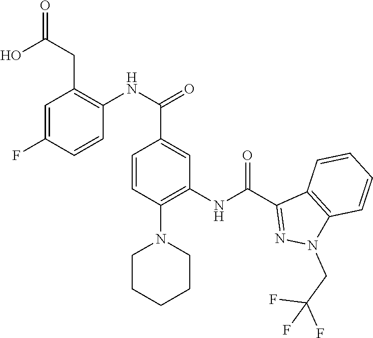 [SMILES: O=C(O)Cc1cc(F)ccc1NC(=O)c1ccc(N2CCCCC2)c(NC(=O)c2nn(CC(F)(F)F)c3ccccc23)c1]

The product was synthesized following General Procedure starting from 1-(2,2,2-trifluoroethyl)-1H-indazole-3-carboxylic acid to afford 2-(5-fluoro-2-(4-(piperidin-1-yl)-3-(1-(2,2,2-trifluoroethyl)-1H-indazole-3-carboxamido)benzamido)phenyl)acetic acid (24.0 mg, 36%) as a white solid. ¹H NMR (300 MHz, DMSO-d₆) δ 12.41 (s, 1H), 10.08 (s, 1H), 10.01 (s, 1H), 8.98 (d, J=2.1 Hz, 1H), 8.39-8.28 (m, 1H), 8.00 (d, J=8.7 Hz, 1H), 7.73 (dd, J=8.4, 2.1 Hz, 1H), 7.68-7.58 (m, 1H), 7.52-7.32 (m, 3H), 7.27-7.04 (m, 2H), 5.67 (q, J=9.0 Hz, 2H), 3.66 (s, 2H), 2.90 (t, J=5.1 Hz, 4H), 1.81 (s, 4H), 1.61 (s, 2H). MS (ESI, m/z): 598 (M+H)⁺.

### Example 2: 2-(2-(3-(1-benzyl-1H-indazole-3-carboxamido)-4-(piperidin-1-yl)benzamido)-5-fluorophenyl)acetic acid

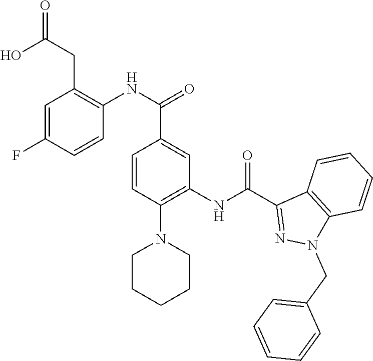 [SMILES: O=C(O)Cc1cc(F)ccc1NC(=O)c1ccc(N2CCCCC2)c(NC(=O)c2nn(Cc3ccccc3)c3ccccc23)c1]

The product was synthesized following General Procedure starting from 1-benzyl-1H-indazole-3-carboxylic acid to afford 2-(2-(3-(1-benzyl-1H-indazole-3-carboxamido)-4-(piperidin-1-yl)benzamido)-5-fluorophenyl)acetic acid (46.7 mg, 51%) as a white solid. ¹H NMR (300 MHz, DMSO-d₆) δ 12.35 (s, 1H), 9.98 (s, 1H), 9.95 (s, 1H), 8.95 (d, J=2.1 Hz, 1H), 8.27 (dd, J=8.1, 1.2 Hz, 1H), 7.98 (d, J=8.4 Hz, 1H), 7.69 (dd, J=8.4, 2.1 Hz, 1H), 7.58-7.49 (m, 1H), 7.46-7.25 (m, 9H), 7.25-7.07 (m, 2H), 5.79 (s, 2H), 3.65 (s, 2H), 2.84 (t, J=5.1 Hz, 4H), 1.67 (d, J=6.6 Hz, 4H), 1.55 (s, 2H). MS (ESI, m/z): 606 (M+H)⁺.

### Example 3: 2-(2-(3-(1-ethyl-1H-indazole-3-carboxamido)-4-(piperidin-1-yl)benzamido)-5-fluorophenyl)acetic acid

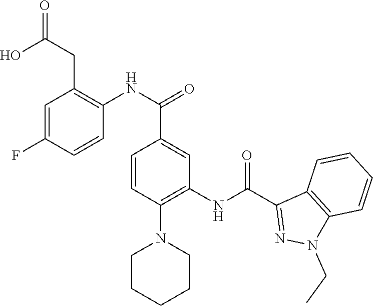 [SMILES: CCn1nc(C(=O)Nc2cc(C(=O)Nc3ccc(F)cc3CC(=O)O)ccc2N2CCCCC2)c2ccccc21]

The product was synthesized following General Procedure starting from I-ethyl-1H-indazole-3-carboxylic acid to afford 2-(2-(3-(1-ethyl-1H-indazole-3-carboxamido)-4-(piperidin-1-yl)benzamido)-5-fluorophenyl)acetic acid (52.5 mg, 54%) as a white solid. ¹H NMR (300 MHz, DMSO-d₆) δ 12.39 (s, 1H), 10.04 (s, 1H), 9.97 (s, 1H), 8.98 (d, J=2.1 Hz, 1H), 8.31-8.22 (m, 1H), 7.85 (d, J=8.7 Hz, 1H), 7.69 (dd, J=8.4, 2.1 Hz, 1H), 7.57-7.46 (m, 1H), 7.45-7.30 (m, 3H), 7.26-7.08 (m, 2H), 4.58 (q, J=7.2 Hz, 2H), 3.66 (s, 2H), 2.97-2.83 (m, 4H), 1.83 (s, 4H), 1.63 (s, 2H), 1.55 (t, J=7.2 Hz, 3H). MS (ESI, m/z): 544 (M+H)⁺.

### Example 4: 2-(5-fluoro-2-(3-(1-isopropyl-1H-indazole-3-carboxamido)-4-(piperidin-1-yl)benzamido)phenyl)acetic acid

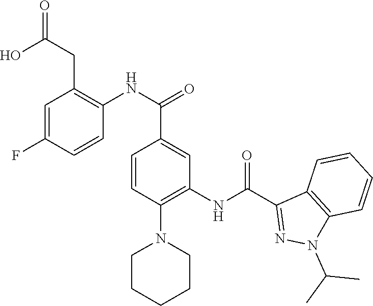 [SMILES: CC(C)n1nc(C(=O)Nc2cc(C(=O)Nc3ccc(F)cc3CC(=O)O)ccc2N2CCCCC2)c2ccccc21]

The product was synthesized following General Procedure starting from 1-isopropyl-1H-indazole-3-carboxylic acid to afford 2-(5-fluoro-2-(3-(1-isopropyl-1H-indazole-3-carboxamido)-4-(piperidin-1-yl)benzamido)phenyl)acetic acid (50.6 mg, 49%) as a white solid. ¹H NMR (300 MHz, DMSO-d₆) δ 12.42 (s, 1H), 10.12 (s, 1H), 10.05 (s, 1H), 9.01 (d, J=2.1 Hz, 1H), 8.27 (dd, J=8.1, 1.1 Hz, 1H), 7.88 (d, J=8.7 Hz, 1H), 7.74-7.65 (m, 1H), 7.56-7.46 (m, 1H), 7.46-7.30 (m, 3H), 7.25-7.08 (m, 2H), 5.26-5.11 (m, 1H), 3.65 (s, 2H), 2.90 (t, J=5.1 Hz, 4H), 1.91-1.78 (m, 4H), 1.70-1.52 (m, 8H). MS (ESI, m/z): 558 (M+H).

### Synthesis of methyl 2-(2-(3-nitro-4-(piperidin-1-yl)benzamido)phenyl)acetate

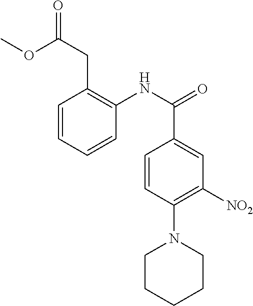 [SMILES: COC(=O)Cc1ccccc1NC(=O)c1ccc(N2CCCCC2)c([N+](=O)[O-])c1]

To a stirred mixture of methyl 2-(2-aminophenyl)acetate (0.66 g, 3.632 mmol, 1.00 equiv), 3-nitro-4-(piperidin-1-yl)benzoic acid (1.0 g, 3.995 mmol, 1.1 equiv) and DIEA (1.41 g, 10.896 mmol, 3.0 equiv) in DMF (10 mL) at ambient temperature was added HATU (2.07 g, 5.448 mmol, 1.5 equiv). The resulting mixture was stirred at this temperature for 1 h and then diluted with EtOAc. The organic layer was washed with brine 3 times, dried over Na₂SO₄ and concentrated under vacuum. The residue was purified by flash chromatography on silica gel (0-60%/6 EtOAc in petroleum ether) to afford methyl 2-(2-(3-nitro-4-(piperidin-1-yl)benzamido)phenyl)acetate (1.4g. 88%) as a red solid. MS (ESI, m/z): 398 (M+H)⁺.

### Synthesis of methyl 2-(2-(3-amino-4-(piperidin-1-yl)benzamido)phenyl)acetate

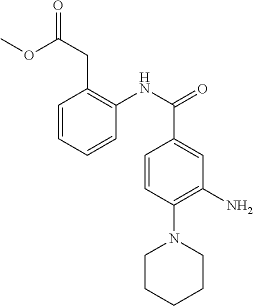 [SMILES: COC(=O)Cc1ccccc1NC(=O)c1ccc(N2CCCCC2)c(N)c1]

A mixture of methyl 2-(2-(3-nitro-4-(piperidin-1-yl)benzamido)phenyl)acetate (1.40 g, 3.523 mmol, 1.00 equiv) and Pd—C(wet, 109%, 270 mg, 10% w/w) in EtOAc (30 mL) was stirred at ambient temperature for 5 h under H₂ (˜3 atm) atmosphere and then filtered. The filtrate was concentrated under vacuum to afford methyl 2-(2-(3-amino-4-(piperidin-1-yl)benzamido)phenyl)acetate (1.2 g, 93%) as a white solid, which was used for the next step without further purification. MS (ESI, m/z): 368 (M+H)⁺.

### Example 5 2-(2-(4-(piperidin-1-yl)-3-(4,5,6,7-tetrahydropyrazolo[1,5-a]pyridine-2-carboxamido)benzamido)phenyl)acetic acid

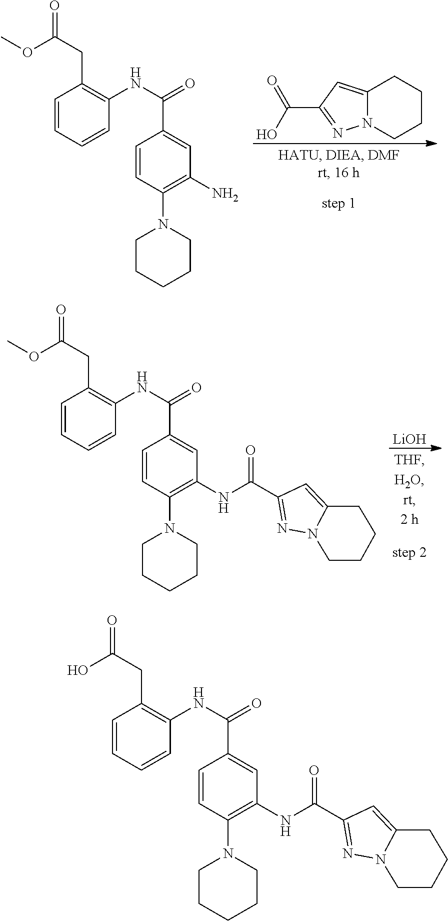 [SMILES: COC(=O)Cc1ccccc1NC(=O)c1ccc(N2CCCCC2)c(N)c1 ; O=C(O)c1cc2n(n1)CCCC2 ; COC(=O)Cc1ccccc1NC(=O)c1ccc(N2CCCCC2)c(NC(=O)c2cc3n(n2)CCCC3)c1 ; "LiOH" ; O=C(O)Cc1ccccc1NC(=O)c1ccc(N2CCCCC2)c(NC(=O)c2cc3n(n2)CCCC3)c1]

### Step 1: Synthesis of methyl 2-(2-(4-(piperidin-1-yl)-3-(4,5,6,7-tetrahydropyrazolo[1,5-a]pyridine-2-carboxamido)benzamido)phenyl)acetate

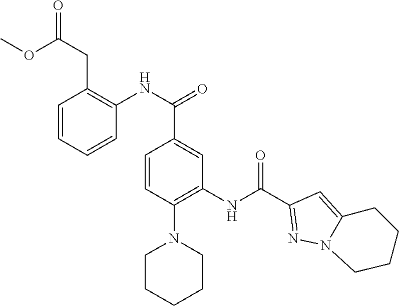 [SMILES: COC(=O)Cc1ccccc1NC(=O)c1ccc(N2CCCCC2)c(NC(=O)c2cc3n(n2)CCCC3)c1]

To a stirred mixture of 4,5,6,7-tetrahydropyrazolo[1,5-a]pyridine-2-carboxylic acid (45 mg, 0.271 mmol, 1.00 equiv), methyl 2-(2-(3-amino-4-(piperidin-1-yl)benzamido)phenyl)acetate (109 mg, 0.298 mmol, 1.10 equiv) and DIEA (105 mg, 0.812 mmol, 3.00 equiv) in DMF (5 mL) at ambient temperature was added HATU (154 mg, 0.406 mmol, 1.50 equiv). The resulting mixture was stirred at this temperature for 1 h and then diluted with EtOAc. The organic layer was washed with brine 3 times, dried over Na₃SO₄ and concentrated under vacuum. The residue was purified by flash chromatography on silica gel (0-80/o EtOAc in petroleum ether) to afford methyl 2-(2-(4-(piperidin-1-yl)-3-(4,5,6,7-tetrahydropyrazolo[1,5-a]pyridine-2-carboxamido)benzamido)phenyl)acetate (87 mg, 62%) as colorless oil. MS (ESI, m/z): 516 (M+H)⁺.

Step 2: Synthesis of 2-(2-(4-(piperidin-1-yl)-3-(4,5,6,7-tetrahydmopymzolo[1,5-a]pyridine-2-carboxamido)benzanido)phenyl)acetic acid

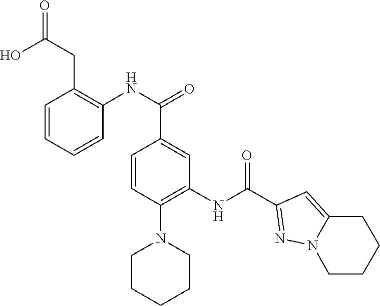 [SMILES: O=C(O)Cc1ccccc1NC(=O)c1ccc(N2CCCCC2)c(NC(=O)c2cc3n(n2)CCCC3)c1]

To a stirred mixture of methyl 2-(2-(4-(piperidin-1-yl)-3-(4,5,6,7-tetrahydropyrazolo[1,5-a]pyridine-2-carboxamido)benzamido)phenyl)acetate (67 mg, 0.130 mmol, 1.00 equiv) in THF (4 mL) and H₂O (1 mL) at ambient temperature was added a solution of LiOH (10 mg, 0.390 mmol, 3.00 equiv) in H₂O (0.5 mL). The resulting mixture solution was stirred at this temperature for 1 h and then concentrated under vacuum. The residue was purified by reverse phase flash chromatography on C18 gel (5-60% acetonitrile in water) to afford 2-(2-(4-(piperidin-1-yl)-3-(4,5,6,7-tetrahydropyrazolo[1,5-a]pyridine-2-carboxamido)benzamido)phenyl)acetic acid (31.2 mg, 48%) as a white solid. ¹H NMR (300 MHz, DMSO-d₆) δ 12.37 (s, 1H), 9.99 (s, 1H), 9.79 (s, 1H), 8.88 (d, J=2.1 Hz, 1H), 7.66 (dd, J=8.4, 2.1 Hz, 1H), 7.50-7.41 (m, 1H), 7.35-7.24 (m, 3H), 7.23-7.13 (m, 1H), 6.52 (s, 1H), 4.16 (t, J=6.0 Hz, 2H), 3.63 (s, 2H), 2.95-2.74 (m, 6H), 2.05-1.93 (m, 2H), 1.87-1.72 (m, 6H), 1.65-1.52 (m, 2H). MS (ESI, m/z): 502 (M+H)˜.

### Example 6: 2-(2-(3-(1-methyl-5-phenyl-1H-pyrazole-3-carboxamido)-4-(piperidin-1-yl)benzamido)phenyl)acetic acid

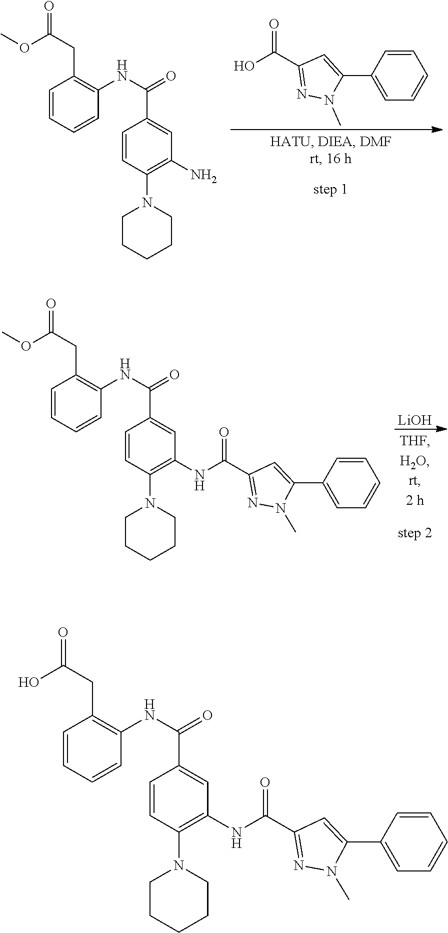 [SMILES: COC(=O)Cc1ccccc1NC(=O)c1ccc(N2CCCCC2)c(N)c1 ; Cn1nc(C(=O)O)cc1-c1ccccc1 ; COC(=O)Cc1ccccc1NC(=O)c1ccc(N2CCCCC2)c(NC(=O)c2cc(-c3ccccc3)n(C)n2)c1 ; "LiOH" ; Cn1nc(C(=O)Nc2cc(C(=O)Nc3ccccc3CC(=O)O)ccc2N2CCCCC2)cc1-c1ccccc1]

### Step 1: Synthesis of methyl 2-(2-(3-(1-methyl-5-phenyl-1H-pyrazole-3-carboxamido)-4-(piperidin-1-yl)benzamido)phenyl)acetate

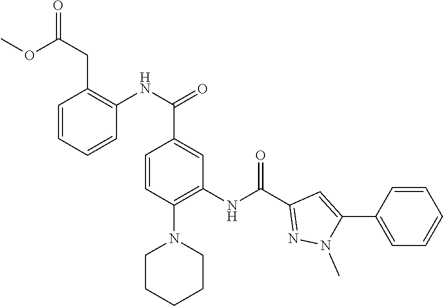 [SMILES: COC(=O)Cc1ccccc1NC(=O)c1ccc(N2CCCCC2)c(NC(=O)c2cc(-c3ccccc3)n(C)n2)c1]

To a stirred mixture of 1-methyl-5-phenyl-1H-pyrazole-3-carboxylic acid (31 mg, 0.161 mmol, 1.00 equiv), methyl 2-(2-(3-amino-4-(piperidin-1-yl)benzamido)phenyl)acetate (59 mg, 0.161 mmol, 1.00 equiv) and DIEA (62 mg, 0.482 mmol, 3.0 equiv) in DMF (3 mL) at ambient temperature was added HATU (92 mg, 0.241 mmol, 1.5 equiv). The resulting mixture was stirred at this temperature for 16 h and then diluted with EtOAc. The organic layer was washed with brine 3 times, dried over Na₂SO₄ and concentrated under vacuum. The residue was purified by flash chromatography on silica gel (0-80% EtOAc in petroleum-ether) to afford methyl 2-(2-(3-(1-methyl-5-phenyl-1H-pyrazole-3-carboxamido)-4-(piperidin-1-yl)benzamido)phenyl)acetate (77 mg, 91%) as a yellow solid. MS (ESI, m/z): 552 (M+H)⁺.

### Step 2: Synthesis of 2-(2-(3-(1-methyl-5-phenyl-1H-pyrazole-3-carboxamido)-4-(piperidin-1-yl)benzamido)phenyl)acetic acid

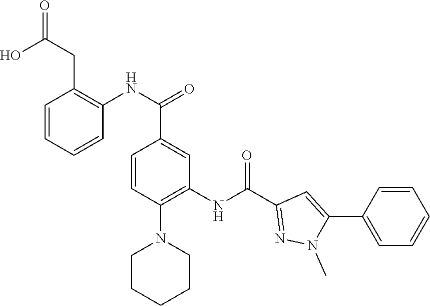 [SMILES: Cn1nc(C(=O)Nc2cc(C(=O)Nc3ccccc3CC(=O)O)ccc2N2CCCCC2)cc1-c1ccccc1]

To a stirred mixture of methyl 2-(2-(3-(1-methyl-5-phenyl-1H-pyrazole-3-carboxamido)-4-(piperidin-1-yl)benzamido)phenyl)acetate (77 mg, 0.140 mmol, 1.00 equiv) in THF (5 mL) and H₂O (1 mL) at ambient temperature was added a solution of LiOH (10 mg, 0.419 mmol, 3.00 equiv) in H₂O (0.5 mL). The resulting mixture solution was stirred at this temperature for 1 h and then concentrated under vacuum. The residue was purified by reverse phase flash chromatography on C18 gel (5-80% acetonitrile in water) to afford 2-(2-(3-(1-methyl-5-phenyl-1H-pyrazole-3-carboxamido)-4-(piperidin-1-yl)benzamido)phenyl)acetic acid (35.7 mg, 48%) as a yellow solid. ¹H NMR (300 MHz, DMSO-d₆) δ 12.36 (brs, 1H), 10.08 (brs, 1H), 9.88 (s, 1H), 8.92 (d, J=2.1 Hz, 1H), 7.69 (dd, J=8.4, 2.1 Hz, 1H), 7.66-7.59 (m, 2H), 7.59-7.43 (m, 4H), 7.37-7.23 (m, 3H), 7.23-7.13 (m, 1H), 6.95 (s, 1H), 3.98 (s, 3H), 3.63 (s, 2H), 2.89 (t, J=5.1 Hz, 4H), 1.89-1.76 (m, 4H), 1.68-1.54 (m, 2H). MS (ESI, m/z): 538 (M+H)⁺.

### Synthesis of 5-nitro-2-(piperidin-1-yl)benzoic acid

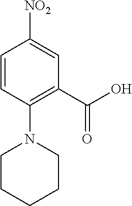 [SMILES: O=C(O)c1cc([N+](=O)[O-])ccc1N1CCCCC1]

A mixture of 2-fluoro-5-nitrobenzoic acid (20 g, 10.8 mmol, 1.00 equiv), piperidine (18.3 g, 21.6 mmol, 2.00 equiv) and DIEA (16.3 g, 21.6 mmol, 2.00 equiv) in DMF (100 mL) was stirred at ambient temperature for 16 h and then poured into ice-water. The precipitated solid was collected by filtration and dried under vacuum to afford 5-nitro-2-(piperidin-1-yl)benzoic acid (crude, 15 g) as a yellow solid, which was used for the next step without further purification. MS (EST, m/z): 251 (M+H)⁺.

### Synthesis of 5-nitro-2-(piperidin-1-yl)-N-(p-tolyl)benzamide

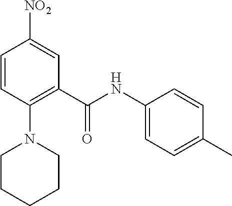 [SMILES: Cc1ccc(NC(=O)c2cc([N+](=O)[O-])ccc2N2CCCCC2)cc1]

To a stirred solution of 5-nitro-2-(piperidin-1-yl)benzoic acid (5.00 g, 19.980 mmol, 1.00 equiv), toluidine (2.14 g, 19.980 mmol, 1.00 equiv) and DIEA (7.75 g, 59.964 mmol, 3.00 equiv) in DMF (50 mL) at ambient temperature was added HATU (11.40 g, 29.970 mmol, 1.50 equiv). The resulting mixture was stirred at this temperature for 1 h and then diluted with EtOAc. The organic layer was washed with brine 3 times, dried over Na₃SO₄ and concentrated under vacuum. The residue was purified by flash chromatography on silica gel (0˜70/a EtOAc in DCM) to afford 5-nitro-2-(piperidin-1-yl)-N-(p-tolyl)benzamide (5 g, 74%) as a brown solid. MS (ESI, m/z): 340 (M+H)⁺.

### Synthesis of 5-amino-2-(piperidin-1-yl)-N-(p-tolyl)benzamide

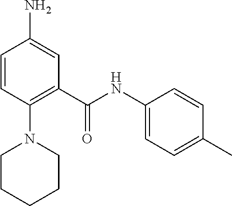 [SMILES: Cc1ccc(NC(=O)c2cc(N)ccc2N2CCCCC2)cc1]

A mixture of N-(4-methylphenyl)-5-nitro-2-(piperidin-1-yl)benzamide (5.00 g, 14.732 mmol, 1.00 equiv) and Pd—C(wet, 10%, 500 mg, 10% w/w) in EtOAc (100 mL) was stirred at ambient temperature for 5 h under H₂ (˜3 atm) atmosphere and then filtered. The filtrate was concentrated under vacuum to afford 5-amino-N-(4-methylphenyl)-2-(piperidin-1-yl)benzamide (4.2 g, 92%) as a white solid, which was used for the next step without further purification. MS (ESI, m/z): 310 (M+H)⁺.

### Example 7: 5-(1-oxo-5-(1H-tetrazol-5-yl)isoindolin-2-yl)-2-(piperidin-1-yl)-N-(p-tolyl)benzamide

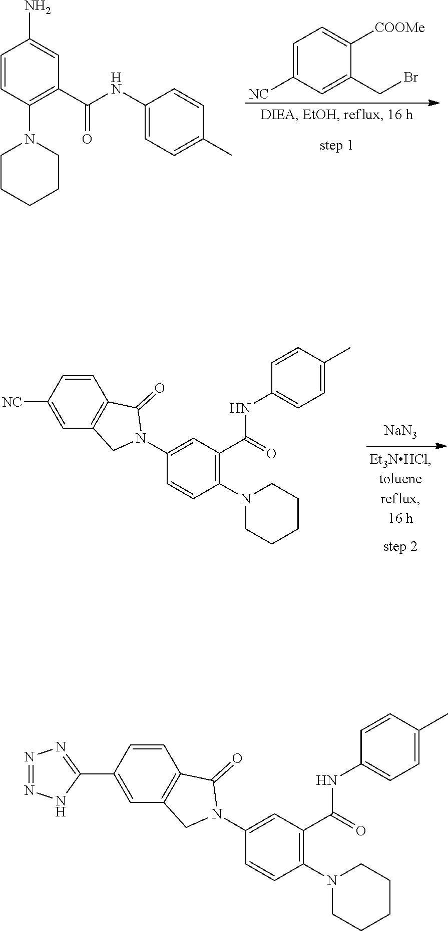 [SMILES: Cc1ccc(NC(=O)c2cc(N)ccc2N2CCCCC2)cc1 ; COC(=O)c1ccc(C#N)cc1CBr ; Cc1ccc(NC(=O)c2cc(N3Cc4cc(C#N)ccc4C3=O)ccc2N2CCCCC2)cc1 ; Cc1ccc(NC(=O)c2cc(N3Cc4cc(-c5nnn[nH]5)ccc4C3=O)ccc2N2CCCCC2)cc1 ; "NaN3"]

### Step 1: Synthesis of 5-(5-cyano-1-Oxoisoindolin-2-yl)-2-(piperidin-1-yl)-N-(p-tolyl)benzamide

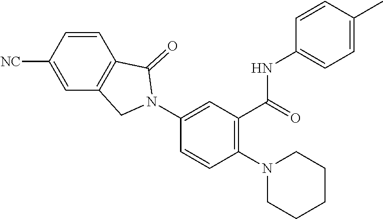 [SMILES: Cc1ccc(NC(=O)c2cc(N3Cc4cc(C#N)ccc4C3=O)ccc2N2CCCCC2)cc1]

A mixture of 5-amino-2-(piperidin-1-yl)-N-(p-tolyl)benzamide (300 mg, 0.970 mmol, 1.00 equiv), methyl 2-(bromomethyl)-4-cyanobenzoate (296 mg, 1.163 mmol, 1.20 equiv) and DIEA (627 mg, 4.848 mmol, 5.00 equiv) in EtOH (10 mL) was refluxed for 32 h and then concentrated under vacuum. The residue was purified by flash chromatography on silica gel (0-40% EtOAc in DCM) to afford 5-(5-cyano-1-oxo-3H-isoindol-2-yl)-N-(4-methylphenyl)-2-(piperidin-1-yl)benzamide (175 mg, 40%) as a white solid. MS (ESI, ma): 451 (M+H)⁺.

### Step 2: Synthesis of 5-(1-oxo-5-(1H-tetrazol-5-yl)isoindolin-2-yl)-2-(piperidin-1-yl)-N-(p-tolyl)benzamide

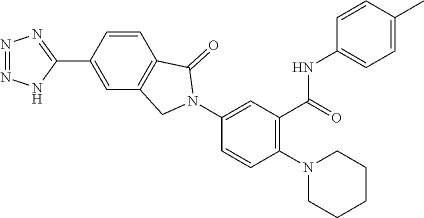 [SMILES: Cc1ccc(NC(=O)c2cc(N3Cc4cc(-c5nnn[nH]5)ccc4C3=O)ccc2N2CCCCC2)cc1]

A mixture of 5-(5-cyano-1-oxo-3H-isoindol-2-yl)-N-(4-methylphenyl)-2-(piperidin-1-yl)benzamide (175 mg, 0.388 mmol, 1.00 equiv), NaN₃ (76 mg, 1.165 mmol, 3.00 equiv) and Et₃N·HCl (214 mg, 1.554 mmol, 4.00 equiv) in toluene (20 mL) was stirred at 110° C. for 20 h. The resulting mixture was basified with NaHCO₃ solution to pH ˜10 and then diluted with brine and extracted with DCM 3 times. The combined organic layers were dried over Na₂SO₄ and concentrated under vacuum. The residue was purified by reverse phase flash chromatography on C18 gel (5-50% acetonitrile in water) to afford 5-(1-oxo-5-(1H-tetrazol-5-yl)isoindolin-2-yl)-2-(piperidin-1-yl)-N-(p-tolyl)benzamide (102.2 mg, 53%) as a white solid. ¹H NMR (400 MHz, DMSO-d₆) δ 11.97 (s, OH), 8.39 (t, J=3.0 Hz, 1H), 8.34 (s, 1H), 8.24-8.18 (m, 1H), 8.11 (dd, J=8.7, 2.7 Hz, 1H), 8.01 (d, J=8.1 Hz, 1H), 7.68 (d, J=8.1 Hz, 2H), 7.46 (dd, J=8.7, 3.3 Hz, 1H), 7.20 (dd, J=8.1 Hz, 2H), 5.18 (s, 2H), 2.97 (t, J=5.1 Hz, 4H), 2.29 (s, 3H), 1.70 (q, J=5.7 Hz, 4H), 1.56 (d, J=7.2 Hz, 2H), 1.23 (s, 1H). MS (ESI, m/z) 494 (M+H)+.

### Step 3: 5-(1-oxo-2-(4-(piperidin-1-yl)-3-(p-tolylcarbamoyl)phenyl)isoindolin-S-yl)tetrazol-1-ide potassium(I)

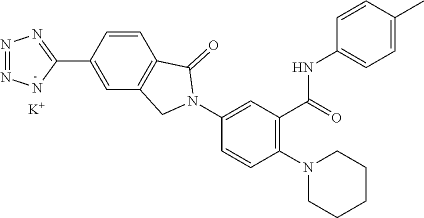 [SMILES: Cc1ccc(NC(=O)c2cc(N3Cc4cc(-c5nnn[nH]5)ccc4C3=O)ccc2N2CCCCC2)cc1 ; [K+]]

To a solution of 5-(1-oxo-5-(1H-tetrazol-5-yl)isoindolin-2-yl)-2-(piperidin-1-yl)-N-(p-tolyl)benzamide (125.00 mg, 0.253 mmol, 1.00 equiv) in MeOH (2 mL) at ambient temperature was added a solution of KOH (15.63 mg, 0.279 mmol, 1.10 equiv) in MeOH (1 mL). The resulting mixture was diluted with H₂O (10 mL) and lyophilized to afford 5-(1-oxo-2-(4-(piperidin-1-yl)-3-(p-tolylcarbamoyl)phenyl)isoindolin-5-yl)tetrazol-1-ide potassium(I) salt (124.5 mg, 92%) as a light yellow solid. ¹H NMR (400 MHz, DMSO-d₆) δ 12.04 (s, 1H), 8.42 (d, J=2.7 Hz, 1H), 8.24 (s, 1H), 8.18 (d, J=8.1 Hz, 1H), 8.11 (dd, J=8.7, 2.7 Hz, 1H), 7.79 (d, J=8.1 Hz, 1H), 7.70 (d, J=8.4 Hz, 2H), 7.46 (d, J=8.7 Hz, 1H), 7.21 (d, J=8.1 Hz, 2H), 5.11 (s, 2H), 3.18 (s, 1H), 2.98 (t, J=5.1 Hz, 4H), 2.30 (s, 3H), 1.79-1.66 (m, 4H), 1.62-1.51 (m, 2H). MS (ESI, m/z): 494 (M+H)⁺.

### Example 8: 5-(6-(1H-tetrazol-5-yl)-2H-indazol-2-yl)-2-(piperidin-1-yl)-N-(p-tolyl)benzamide

### Step 1: Synthesis of S-(6-cyano-2H-indazol-2-yl)-2-(piperidin-yl)-N-(p-tolyl)benzamide

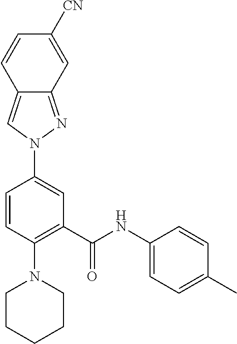 [SMILES: Cc1ccc(NC(=O)c2cc(-n3cc4ccc(C#N)cc4n3)ccc2N2CCCCC2)cc1]

A mixture of 5-amino-2-(piperidin-1-yl)-N-(p-tolyl)benzamide (200 mg, 0.646 mmol, 1.00 equiv), and 4-formyl-3-nitrobenzonitrile (125 mg, 0.711 mmol, 1.10 equiv) in i-PrOH (5 mL) was stirred at 80° C. for 4 h under N₂ atmosphere. To this at ambient temperature was added P(t-Bu)₃ (392 mg, 1.939 mmol, 3.0 equiv). The resulting solution was stirred at 80° C. for another 16 h and then concentrated under vacuum. The residue was purified by reverse phase flash chromatography on C18 gel (5-60% acetonitrile in water) to afford 5-(6-cyano-2H-indazol-2-yl)-2-(piperidin-1-yl)-N-(p-tolyl)benzamide (251 mg, 89%) as a yellow solid. MS (ESI, m/z): 436 (M+H)⁺.

### Step 2: Synthesis of S-(6-(1H-tetrazol-S-yl)-2H-indazol-2-yl)-2-(piperidin-1-yl)-N-(p-tolyl)benzamide

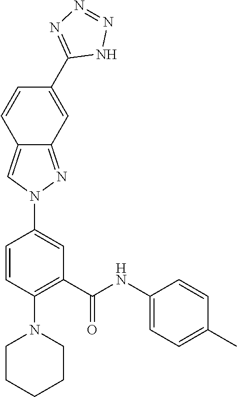 [SMILES: Cc1ccc(NC(=O)c2cc(-n3cc4ccc(-c5nnn[nH]5)cc4n3)ccc2N2CCCCC2)cc1]

A mixture of 5-(6-cyano-2H-indazol-2-yl)-2-(piperidin-1-yl)-N-(p-tolyl)benzamide (250 mg, 0.574 mmol, 1.00 equiv), NaN₃ (112 mg, 1.722 mmol, 3.00 equiv) and Et₃N·HCl (158 mg, 1.148 mmol, 2.00 equiv) in toluene (10 mL) was stirred at 110° C. for 32 h. The resulting mixture was basified with NaHCO₃ solution to pH ˜10 and then diluted with brine and extracted with DCM 3 times. The combined organic layers were dried over Na₂SO₄ and concentrated under vacuum. The residue was purified by reverse phase flash chromatography on C18 gel (5-100% acetonitrile in water) to afford 5-(6-(1H-tetrazol-5-yl)-2H-indazol-2-yl)-2-(piperidin-1-yl)-N-(p-tolyl)benzamide (26.4 mg, 10%) as a yellow solid. ¹H NMR (300 MHz, DMSO-d₆) δ 11.38 (s, 1H), 9.25 (d, J=1.2 Hz, 1H), 8.47 (d, J=2.7 Hz, 1H), 8.41 (d, J=1.2 Hz, 1H), 8.20 (dd, J=8.7, 2.7 Hz, 1H), 7.97 (dd, J=8.7, 0.9 Hz, 1H), 7.76 (dd, J=8.7, 1.5 Hz, 1H), 7.72-7.64 (m, 2H), 7.48 (d, J=8.7 Hz, 1H), 7.19 (d, J=8.1 Hz, 2H), 3.10-2.96 (m, 4H), 2.28 (s, 3H), 1.76-1.60 (m, 4H), 1.59-1.45 (m, 2H). MS (ESI, m/z): 479 (M+H)+.

### Example 9: 5-((4-(5-oxo-4,5-dihydro-1,2,4-thiadiazol-3-yl)benzyl)amino)-2-(piperidin-1-yl)-N-(p-tolyl)benzamide

### Step 1: Synthesis of 5-((4-cyanobenzyl)amino)-2-(piperidin-1-yl)-N-(p-tolyl)benzamide

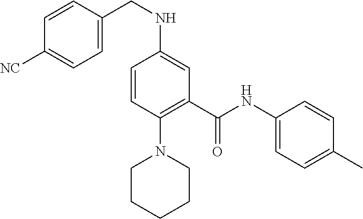 [SMILES: Cc1ccc(NC(=O)c2cc(NCc3ccc(C#N)cc3)ccc2N2CCCCC2)cc1]

A mixture of 5-amino-2-(piperidin-1-yl)-N-(p-tolyl)benzamide (640 mg, 2.068 mmol, 1.00 equiv) and 4-formylbenzonitrile (298 mg, 2.275 mmol, 1.10 equiv) in EtOH (20 mL) was stirred at ambient temperature for 16 h. To this was added NaBH₃CN (273 mg, 4.344 mmol, 2.10 equiv). The resulting mixture was stirred for further 2 h and then quenched with HCl (1M). The resulting mixture was basified with NaHCO₃ solution to pH ˜9 and then diluted with brine and extracted with DCM 3 times. The combined organic layers were dried over Na₂SO₄ and concentrated under vacuum. The residue was purified by flash chromatography on silica gel (0-40% EtOAc in DCM) to afford 5-((4-cyanobenzyl)amino)-2-(piperidin-1-yl)-N-(p-tolyl)benzamide (650 mg, 74%) as a white solid. MS (EST, m/z): 425 (M+H)⁺.

### Step 2: Synthesis of(Z)-5-((4-(N′-hydroxyarbamimidoyl)benzyl)amino)-2-(piperidin-1-yl)-N-(p-tolyl)benzamide

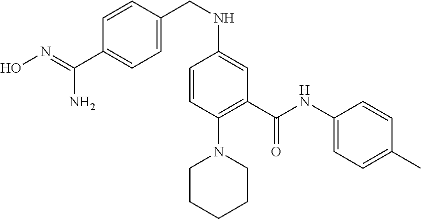 [SMILES: Cc1ccc(NC(=O)c2cc(NCc3ccc(/C(N)=N/O)cc3)ccc2N2CCCCC2)cc1]

A mixture of 5-((4-cyanobenzyl)amino)-2-(piperidin-1-yl)-N-(p-tolyl)benzamide (500 mg, 1.178 mmol, 1.00 equiv), NH₂OH·HCl (164 mg, 2.355 mmol, 2.00 equiv) and TEA (357.52 mg, 3.533 mmol, 3 equiv) in EtOH (20 mL) was stirred at 90° C. for 16 h and then concentrated under vacuum. The residue was purified by reverse phase flash chromatography on C18 gel (5-80% acetonitrile in water) to afford (Z)-5-((4-(N′-hydroxycarbamimidoyl)benzyl)amino)-2-(piperidin-1-yl)-N-(p-tolyl)benzamide (300 mg, 56%) as a white solid. MS (ESI, m. z): 458 (M+H)⁺.

### Step 3: Synthesis of 5-((4-(5-oxo-4,5-dihydro-,2,4-thiadiazol-3-v)benzyl)amino)-2-(piperidin-1-yl)-N-(p-tolyl)benzamide

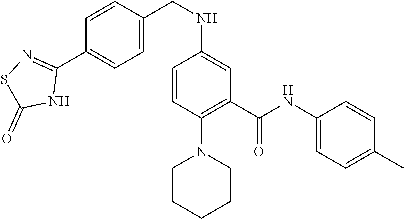 [SMILES: Cc1ccc(NC(=O)c2cc(NCc3ccc(-c4nsc(=O)[nH]4)cc3)ccc2N2CCCCC2)cc1]

A mixture of (Z)-5-((4-(N′-hydroxycarbamimidoyl)benzyl)amino)-2-(piperidin-1-yl)-N-(p-tolyl)benzamide (200 mg, 0.437 mmol, 1.00 equiv) and CDI (86 mg, 0.481 mmol, 1.10 equiv) in THF (5.00 mL) was stirred at ambient temperature for 2 h. To this at 0° C. was added dropwise a solution of BF₃·Et₂₀ (310 mg, 2.185 mmol, 5.00 equiv) in THF (1 mL). The resulting mixture was stirred at ambient temperature for another 2 h and then quenched with water and extracted with EtOAc 3 times. The combined organic layers were dried over Na₂SO₄ and concentrated under vacuum. The residue was purified by reverse phase flash chromatography on C18 gel (5-70% acetonitrile in water) and further purified by Prep-HPLC to afford 5-((4-(5-oxo-4,5-dihydro-1,2,4-thiadiazol-3-yl)benzyl)amino)-2-(piperidin-1-yl)-N-(p-tolyl)benzamide (6 mg, 3%) as a yellow solid. ¹H NMR (300 MHz, DMSO-d₆) δ 13.34 (s, 1H), 13.01 (s, 1H), 7.89 (d, J=8.1 Hz, 2H), 7.60 (d, J=8.4 Hz, 2H), 7.50 (d, J=8.1 Hz, 2H), 7.33 (d, J=3.0 Hz, 1H), 7.18 (t, J=8.4 Hz, 3H), 6.70 (dd, J=8.7, 3.0 Hz, 1H), 6.56 (t, J=6.3 Hz, 1H), 4.36 (d, J=5.7 Hz, 2H), 2.82 (t, J=5.1 Hz, 4H), 2.27 (s, 3H), 1.69 (brs, 4H), 1.55 (brs, 2H). MS (ESI, m/z): 500 (M+H)⁺.

### Example 10: N-(5-((4-(1H-tetrazol-5-yl)phenyl)carbamoyl)-2-(piperidin-1-yl)phenyl)-1-(2,2,2-trifluoroethyl)-1H-indazole-3-carboxamide

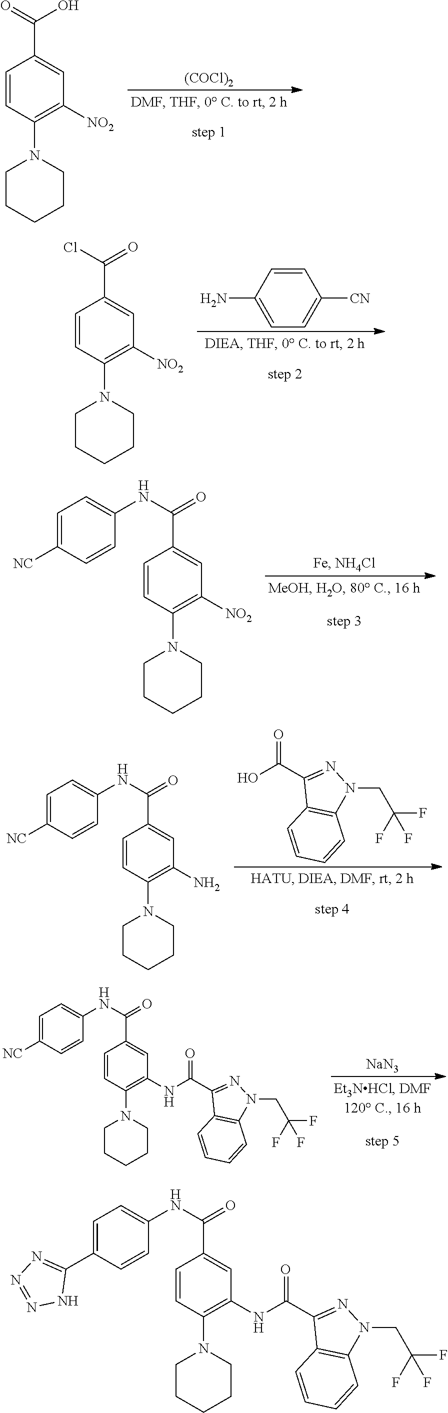

### Step 1: Synthesis of 3-nitro-4-(piperidin-1-yl)benzoyl Chloride

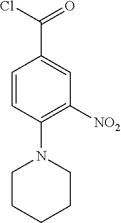 [SMILES: O=C(Cl)c1ccc(N2CCCCC2)c([N+](=O)[O-])c1]

To a stirred mixture of 3-nitro-4-(piperidin-1-yl)benzoic acid (500 mg, 1.998 mmol, 1.00 equiv) in THF (10 mL) at 0° C. was added (COCl)₂ (761 mg, 5.994 mmol, 3.00 equiv) and DMF (1.5 mg, 0.01 equiv). The resulting mixture was stirred at ambient temperature for 2 h and then concentrated under vacuum to afford 3-nitro-4-(piperidin-1-yl)benzoyl chloride (crude, 538 mg) brown oil, which was used for the next step without further purification.

### Step 2: Synthesis of N-(4-cyanophenyl)-3-nitro-4-(piperidin-1-yl)benzamide

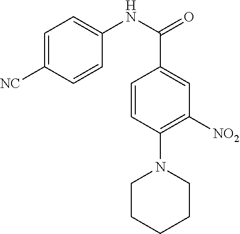 [SMILES: N#Cc1ccc(NC(=O)c2ccc(N3CCCCC3)c([N+](=O)[O-])c2)cc1]

To a stirred mixture of 4-aminobenzonitrile (236 mg, 2.0 mmol, 1.00 equiv) in THF (10 mL) at 0° C. was added dropwise a solution of 3-nitro-4-(piperidin-1-yl)benzoyl chloride (538 mg, 2.0 mmol, 1.00 equiv) in THF (5 mL). The resulting mixture was stirred at ambient temperature for 2 h and then concentrated under vacuum. The residue was purified by flash chromatography on silica gel (0-80% EtOAc in petroleum ether) to afford N-(4-cyanophenyl)-3-nitro-4-(piperidin-1-yl)benzamide (500 mg, 71%) as brown oil. MS (ESI, m/z): 351 (M+H)⁺.

### Step 3: Synthesis of 3-amino-N-(4-cyanophenyl)-4-(piperidin-1-yl)benzamide

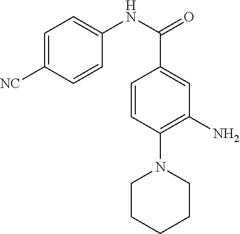 [SMILES: N#Cc1ccc(NC(=O)c2ccc(N3CCCCC3)c(N)c2)cc1]

A mixture of N-(4-cyanophenyl)-3-nitro-4-(piperidin-1-yl)benzamide (500 mg, 1.427 mmol, 1.00 equiv), NH₄Cl (763 mg, 14.270 mmol, 10 equiv) and Fe power (398 mg, 7.135 mmol, 5 equiv) in EtOH (16 mL) and H₂O (2 mL) was stirred at 80° C. for 2 h and then filtered. The filtrate was concentrated vacuum. The residue was purified by flash chromatography on silica gel (0-10% MeOH in DCM) to afford 3-amino-N-(4-cyanophenyl)-4-(piperidin-1-yl)benzamide (170 mg, 37%) as a yellow solid. MS (EST, m/z): 321 (M+H)⁺.

### Step 4: Synthesis of N—(S-((4-cyanophenyl)carbamoyl)-2-(piperidin-1-yl)phenyl)-1-(2,2,2-trifluoroethyl)-1H-indazole-3-carboxamide

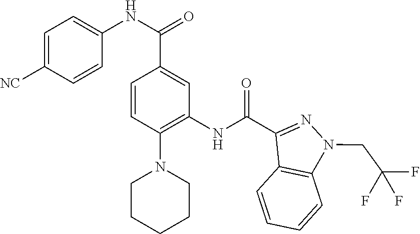 [SMILES: N#Cc1ccc(NC(=O)c2ccc(N3CCCCC3)c(NC(=O)c3nn(CC(F)(F)F)c4ccccc34)c2)cc1]

To a stirred mixture of 3-amino-N-(4-cyanophenyl)-4-(piperidin-1-yl)benzamide (170 mg, 0.531 mmol, 1.00 equiv), 1-(2,2,2-trifluoroethyl)-1H-indazole-3-carboxylic acid (143 mg, 0.584 mmol, 1.10 equiv) and DIEA (206 mg, 1.592 mmol, 3.0 equiv) in DMF (5 mL) at ambient temperature was added HATU (303 mg, 0.796 mmol, 1.5 equiv). The resulting mixture was stirred at this temperature for 2 h and then diluted with EtOAc. The organic layer was washed with brine 3 times, dried over Na₂SO₄ and concentrated under vacuum. The residue was purified by flash chromatography on silica gel (0-80% EtOAc in petroleum ether) to afford N-(5-((4-cyanophenyl)carbamoyl)-2-(piperidin-1-yl)phenyl)-1-(2,2,2-trifluoroethyl)-1H-indazole-3-carboxamide (240 mg, 83%) as a yellow solid. MS (ESI, m/z): 547 (M+H)⁺.

### Step 5: Synthesis of N-(5-((4-(1H-tetrazol-1-yl)phenyl)carbamoyl)-2-(piperidin-yl)phenyl)-1-(2,2,2-trifluoroethyl)-1H-indazole-3-carboxamide

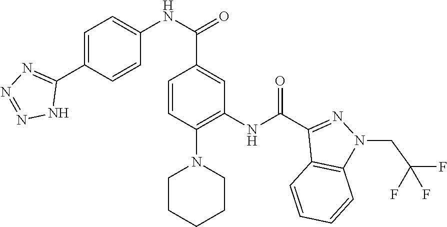 [SMILES: O=C(Nc1ccc(-c2nnn[nH]2)cc1)c1ccc(N2CCCCC2)c(NC(=O)c2nn(CC(F)(F)F)c3ccccc23)c1]

A mixture of N-(5-((4-cyanophenyl)carbamoyl)-2-(piperidin-1-yl)phenyl)-1-(2,2,2-trifluoroethyl)-1H-indazole-3-carboxamide (240 mg, 0.439 mmol, 1.00 equiv), NaN₃ (86 mg, 1.317 mmol, 3.00 equiv) and Et₃N·HCl (242 mg, 1.756 mmol, 4 equiv) in DMF (10.00 mL) was stirred at 120° C. for 32 h. The resulting mixture was basified with NaHCO₃ solution to pH 10, diluted with brine and extracted with DCM 3 times. The combined organic layers were dried over Na₂SO₄ and concentrated under vacuum. The residue was purified by reverse phase flash chromatography on C18 gel (5-80% acetonitrile in water) to afford N-(5-((4-(1H-tetrazol-5-yl)phenyl)carbamoyl)-2-(piperidin-1-yl)phenyl)-1-(2,2,2-trifluoroethyl)-1H-indazole-3-carboxamide (113 mg, 44%) as a white solid. ¹H NMR (300 MHz, DMSO-d₆) δ 10.53 (s, 1H), 10.01 (s, 1H), 8.98 (d, J=2.1 Hz, 1H), 8.37-8.27 (m, 1H), 8.07-7.94 (m, 5H), 7.74 (dd, J=8.4, 2.1 Hz, 1H), 7.67-7.55 (m, 1H), 7.49-7.34 (m, 2H), 5.66 (q, J=9.0 Hz, 2H), 2.89 (t, J=5.1 Hz, 4H), 1.87-1.73 (m, 4H), 1.65-1.54 (m, 2H). MS (ESI, m/z): 590(M+H)⁺.

### Example 11: N-(5-((2-fluoro-5-(1H-tetrazol-5-yl)phenyl)carbamoyl)-2-(piperidin-1-yl)phenyl)-1-methyl-1H-indazole-3-carboxamide

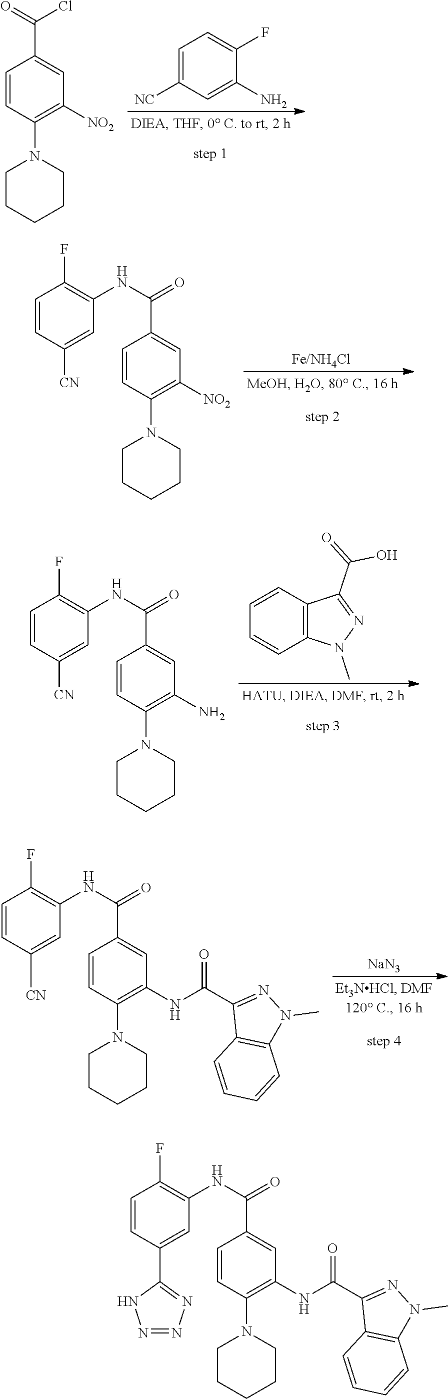

### Step 1: Synthesis of N-(5-cyano-2-fluorophenyl)-3-nitro-4-(piperidin-1-yl)benzamide

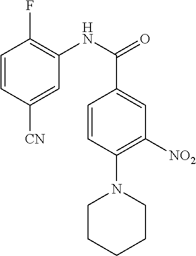 [SMILES: N#Cc1ccc(F)c(NC(=O)c2ccc(N3CCCCC3)c([N+](=O)[O-])c2)c1]

To a stirred solution of 3-amino-4-fluorobenzonitrile (979 mg, 7.193 mmol, 1.2 equiv) and TEA (1.82 g, 17.982 mmol, 3.00 equiv) in THF (15 mL) at 0° C. was added dropwise a solution of 3-nitro-4-(piperidin-1-yl)benzoyl chloride (1.61 g, 5.994 mmol, 1.00 equiv) in THF (5 mL). The resulting mixture was stirred at ambient temperature for 2 h and then quenched with water and extracted with EtOAc 3 times. The combined organic layers were dried over Na₂SO₄ and concentrated under vacuum. The residue was purified by flash chromatography on silica gel (0-80% EtOAc in petroleum ether) to afford N-(5-cyano-2-fluorophenyl)-3-nitro-4-(piperidin-1-yl)benzamide (680 mg, 31%) as a yellow solid. MS (EST, m/z): 369 (M+H)⁺.

### Step 2: Synthesis of 3-amino-N—(S-cyano-2-fluorophenyl)-4-(piperidin-yl)benzamide

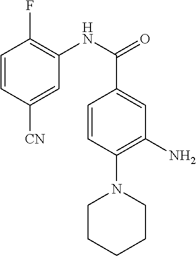 [SMILES: N#Cc1ccc(F)c(NC(=O)c2ccc(N3CCCCC3)c(N)c2)c1]

A mixture of N-(5-cyano-2-fluorophenyl)-3-nitro-4-(piperidin-1-yl)benzamide (680 mg, 1.846 mmol, 1.00 equiv), iron power (515 mg, 9.230 mmol, 5 equiv) and NH₄Cl (987 mg, 18.460 mmol, 10.00 equiv) in MeOH (20.00 mL) and H₂O (2.50 mL) was stirred at 80° C. for 16 h and then filtered. The filter cake was washed with MeOH and the filtrate was concentrated under vacuum. The residue was purified by flash chromatography on silica gel (0-10% MeOH in DCM) to afford 3-amino-N-(5-cyano-2-fluorophenyl)-4-(piperidin-1-yl)benzamide (500 mg, 80%) as a brown solid. MS (ESI, m/z): 339 (M+H)⁺.

### Step 3: Synthesis of N-(5-((5-cyano-2-fluorophenyl)carbamoyl)-2-(piperidin-1-yl)phenyl)-1-methyl-1H-indazole-3-carboxamide

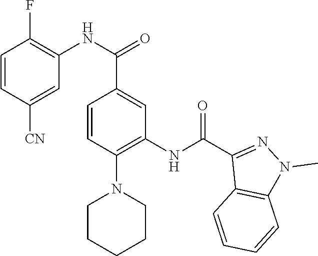 [SMILES: Cn1nc(C(=O)Nc2cc(C(=O)Nc3cc(C#N)ccc3F)ccc2N2CCCCC2)c2ccccc21]

To a stirred mixture of 3-amino-N-(5-cyano-2-fluorophenyl)-4-(piperidin-1-yl)benzamide (250 mg, 0.739 mmol, 1.00 equiv), 1-methylindazole-3-carboxylic acid (156 mg, 0.887 mmol, 1.2 equiv) and DIEA (382 mg, 2.955 mmol, 4 equiv) in DMF (10 mL) at ambient temperature was added HATU (421 mg, 1.108 mmol, 1.5 equiv). The resulting mixture was stirred at this temperature for 2 h and then diluted with EtOAc. The organic solution was washed with brine 3 times, dried over Na₂SO₄ and concentrated under vacuum. The residue was purified by flash chromatography on silica gel (0-10/MeOH in DCM) to afford N-(5-((5-cyano-2-fluorophenyl)carbamoyl)-2-(piperidin-1-yl)phenyl)-1-methyl-1H-indazole-3-carboxamide (270 mg, 73%) as a light yellow solid. MS (EST, m/z): 497 (M+H)⁺.

### Step 4: Synthesis of N-(5-((2-fluoro-5-(1H-tetrazol-5-yl)phenyl)carbamoyl)-2-(piperidin-1-yl)phenyl)-1-methyl-1H-indazole-3-carboxamide

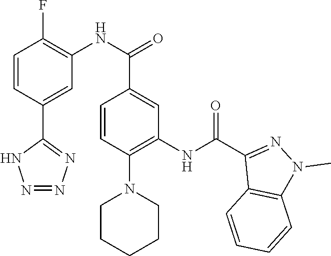 [SMILES: Cn1nc(C(=O)Nc2cc(C(=O)Nc3cc(-c4nnn[nH]4)ccc3F)ccc2N2CCCCC2)c2ccccc21]

A mixture of N-(5-((5-cyano-2-fluorophenyl)carbamoyl)-2-(piperidin-1-yl)phenyl)-1-methyl-1H-indazole-3-carboxamide (243 mg, 0.489 mmol, 1.00 equiv), NaN₃ (95 mg, 1.468 mmol, 3.00 equiv) and Et₃N·HCl (268 mg, 1.958 mmol, 4.00 equiv) in DMF (10 mL) was stirred at 120° C. for 16 h. The resulting mixture was basified with NaHCO₃ solution to pH ˜10, diluted with brine and extracted with DCM 3 times. The combined organic layers were dried over Na₂SO₄ and concentrated under vacuum. The residue was purified by reverse phase flash chromatography on C18 gel (5-80% acetonitrile in water) to afford N-(5-((2-fluoro-5-(1H-tetrazol-5-yl)phenyl)carbamoyl)-2-(piperidin-1-yl)phenyl)-1-methyl-1H-indazole-3-carboxamide (50 mg, 19%) as an off-white solid. ¹H NMR (300 MHz, DMSO-d₆) δ 10.36 (s, 1H), 10.00 (s, 1H), 9.06 (d, J=2.1 Hz, 1H), 8.39 (dd, J=7.2, 2.1 Hz, 1H), 8.33-8.22 (m, 1H), 8.00-7.90 (m, 1H), 7.87-7.74 (m, 2H), 7.64-7.51 (m, 2H), 7.44-7.33 (m, 2H), 4.23 (s, 3H), 2.99-2.90 (m, 4H), 1.93-1.79 (s, 4H), 1.73-1.58(m. 2H). MS (ESI, m/z): 540 (M+H)⁺.

### Example 12: N-(5-((4-(1H-tetrazol-5-yl)phenyl)carbamoyl)-2-(piperidin-1-yl)phenyl)-6-fluoro-1-methyl-11H-indazole-3-carboxamide

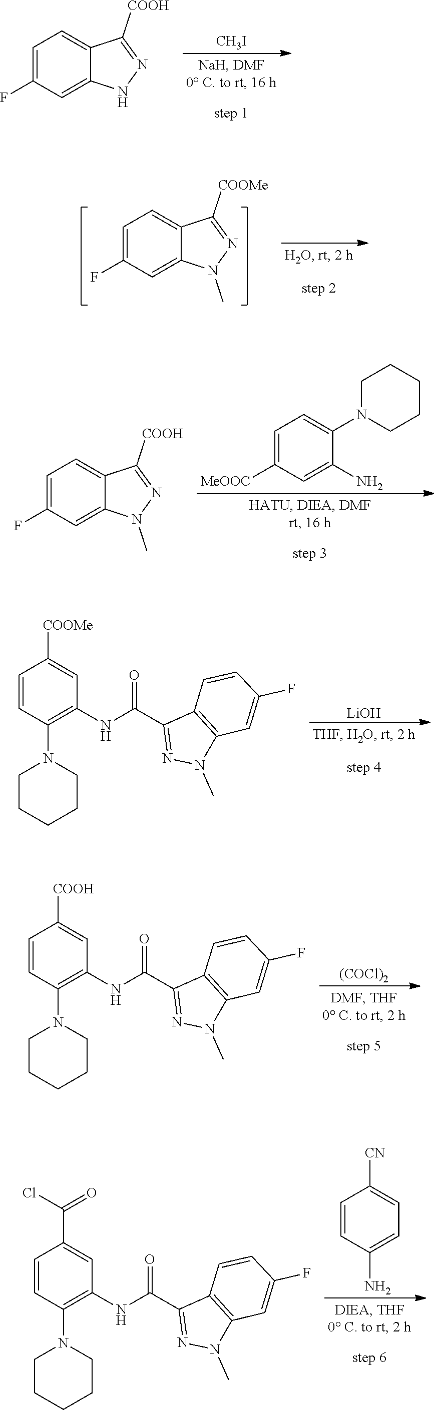

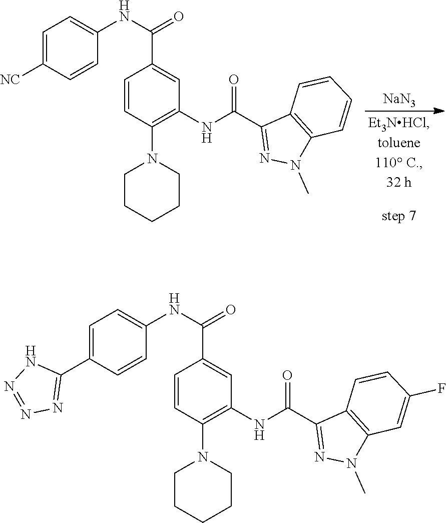 [SMILES: Cn1nc(C(=O)Nc2cc(C(=O)Nc3ccc(C#N)cc3)ccc2N2CCCCC2)c2ccccc21 ; Cn1nc(C(=O)Nc2cc(C(=O)Nc3ccc(-c4nnn[nH]4)cc3)ccc2N2CCCCC2)c2ccc(F)cc21 ; "NaN3"]

### Step 1: Synthesis of 6-fluoro-1-methyl-1H-indazole-3-carboxylic acid

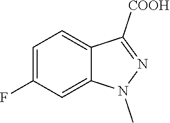

To a stirred mixture of 6-fluoro-1H-indazole-3-carboxylic acid (280 mg, 1.560 mmol, 1.00 equiv) in DMF (10 mL) at 0° C. was added NaH (60% in mineral oil, 187 mg, 4.680 mmol, 3.00 equiv). The resulting mixture was stirred at this temperature for 1 h and to this was added CH (487 mg, 3.432 mmol, 2.20 equiv). The resulting mixture was stirred at ambient temperature for another 16 h and then cooled down to 0° C. To this was added H₂O (10 mL). The resulting mixture was stirred at ambient temperature for further 1 h and then acidified at 0° C. with HCl (1M) to pH ˜6 and extracted with DCM 3 times. The combined organic layers were dried over Na₂SO₄ and concentrated under vacuum. The residue was purified by reverse phase flash chromatography on C18 gel (5-40% acetonitrile in water) to afford 6-fluoro-1-methyl-1H-indazole-3-carboxylic acid (170 mg, 56%) as a white solid. MS (ESI, m z): 195 (M+H)⁺.

### Step 2: Synthesis of methyl 3-(6-fluoro-1-methyl-1H-indazole-3-carboxamido)-4-(piperidin-1-yl)benzoate

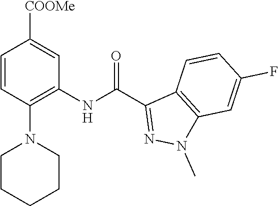 [SMILES: COC(=O)c1ccc(N2CCCCC2)c(NC(=O)c2nn(C)c3cc(F)ccc23)c1]

To a stirred solution of 6-fluoro-1-methyl-1H-indazole-3-carboxylic acid (170 mg, 0.876 mmol, 1.00 equiv), methyl 3-amino-4-(piperidin-1-yl)benzoate (205 mg, 0.876 mmol, 1.00 equiv) and DIEA (339 mg, 2.628 mmol, 3.00 equiv) in DMF (2 mL) at ambient temperature was added HATU (499 g, 1.314 mmol, 1.50 equiv). The resulting mixture was stirred at this temperature for 1 h and then diluted with EtOAc. The organic layer was washed with brine 3 times, dried over Na₂SO₄ and concentrated under vacuum. The residue was purified by flash chromatography on silica gel (0-50% EtOAc in DCM) to afford methyl 3-(6-fluoro-1-methyl-1H-indazole-3-carboxamido)-4-(piperidin-1-yl)benzoate (293 mg, 82%) as a brown solid.

MS (ESI, m/z): 411 (M+H)⁺.

### Step 3: Synthesis of 3-(6-fluoro-1-methyl-1H-indazole-3-carboxamido)-4-(piperidin-1-yl)benzoic acid

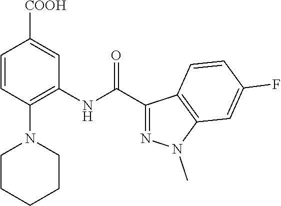 [SMILES: Cn1nc(C(=O)Nc2cc(C(=O)O)ccc2N2CCCCC2)c2ccc(F)cc21]

To a stirred mixture of methyl 3-(6-fluoro-1-methyl-1H-indazole-3-carboxamido)-4-(piperidin-1-yl)benzoate (293 mg, 0.715 mmol, 1.00 equiv) in THF (6 mL) and H₂O (1 mL) at ambient temperature was added a solution of LiOH (51 mg, 2.14 mmol, 3.00 equiv) in H₂O (1 mL). The resulting mixture solution was stirred at this temperature for 1 h and then concentrated under vacuum. The residue was purified by reverse phase flash chromatography on C18 gel (5-50% acetonitrile in water) to afford 3-(6-fluoro-1-methyl-1H-indazole-3-carboxamido)-4-(piperidin-1-yl)benzoic acid (240 mg, 85%) as a brown solid. MS (ESI, m/z): 397 (M+H)⁺.

### Step 4: Synthesis of 3-(6-fluoro-1-methyl-1H-indazole-3-carboxamido)-4-(piperidin-1-yl)benzoyl chloride

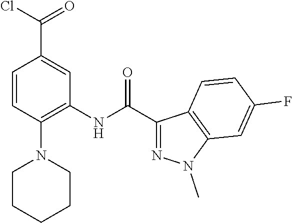 [SMILES: Cn1nc(C(=O)Nc2cc(C(=O)Cl)ccc2N2CCCCC2)c2ccc(F)cc21]

To a stirred mixture of 3-(6-fluoro-1-methyl-1H-indazole-3-carboxamido)-4-(piperidin-1-yl)benzoic acid (130 mg, 0.328 mmol, 1.00 equiv) in THF (10 mL) at 0° C. was added (COCl)₂ (761 mg, 5.994 mmol, 3.00 equiv) and DMF (1.5 mg, 0.01 equiv). The resulting mixture was stirred at ambient temperature for 2 h and then concentrated under vacuum to afford 3-(6-fluoro-1-methyl-1H-indazole-3-carboxamido)-4-(piperidin-1-yl)benzoyl chloride (crude, 140 mg) as brown oil, which was used for the next step without for further purification.

### Step 5: Synthesis of N-(5-((4-cyanophenyl)carbamoyl)-2-(piperidin-1-yl)phenyl)-methyl-1-indazole-3-carboxamide

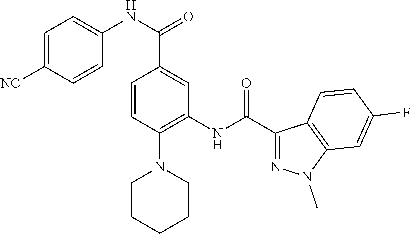 [SMILES: Cn1nc(C(=O)Nc2cc(C(=O)Nc3ccc(C#N)cc3)ccc2N2CCCCC2)c2ccc(F)cc21]

To a stirred solution of 4-aminobenzonitrile (60 mg, 0.506 mmol, 1.5 equiv) and DIEA (131 mg, 1.012 mmol, 3.00 equiv) in THF (5 mL) at 0° C. was added dropwise a solution of 3-(6-fluoro-1-methyl-1H-indazole-3-carboxamido)-4-(piperidin-1-yl)benzoyl chloride (140 mg, 0.329 mmol, 1.00 equiv) in THF (2 mL). The resulting mixture was stirred at ambient temperature for 2 h and then quenched with water and extracted with EtOAc 3 times. The combined organic layers were dried over Na₂SO₄ and concentrated under vacuum. The residue was purified by flash chromatography on silica gel (0-60% EtOAc in DCM) to afford N-(5-((4-cyanophenyl)carbamoyl)-2-(piperidin-1-yl)phenyl)-1-methyl-1H-indazole-3-carboxamide (108 mg, 67%) as an off-white solid. MS (ESI, m/z): 497 (M+H)⁺.

### Step 6: Synthesis of N—(S-((4-(JH-tetrazol-S-yl)phenyl)carbamoyl)-2-(piperidin-1-yl)phenyl)-6-fluoro-1-methyl-1H-indazole-3-carboxamide

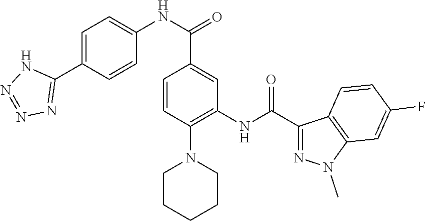 [SMILES: Cn1nc(C(=O)Nc2cc(C(=O)Nc3ccc(-c4nnn[nH]4)cc3)ccc2N2CCCCC2)c2ccc(F)cc21]

A mixture of N-(5-((4-cyanophenyl)carbamoyl)-2-(piperidin-1-yl)phenyl)-1-methyl-1H-indazole-3-carboxamide (108 mg, 0.218 mmol, 1.00 equiv), NaN₃ (85 mg, 1.305 mmol, 6.00 equiv) and Et₃N·HCl (180 mg, 1.305 mmol, 6.00 equiv) in toluene (20 mL) was refluxed for 32 h. The resulting mixture was basified with NaHCO₃ solution to pH ˜10, diluted with brine, and extracted with DCM 3 times. The combined organic layers were dried over Na₂SO₄ and concentrated under vacuum. The residue was purified by reverse phase flash chromatography on C18 gel (5-65% acetonitrile in water) to afford N-(5-((4-(1H-tetrazol-5-yl)phenyl)carbamoyl)-2-(piperidin-1-yl)phenyl)-6-fluoro-1-methyl-1H-indazole-3-carboxamide (32.1 mg, 26%) as an off-white solid. ¹H NMR (300 MHz, DMSO-d₆) δ 10.54 (s, 1H), 9.98 (s, 1H), 8.99 (d, J=2.1 Hz, 1H), 8.30-8.22 (m, 1H), 8.03 (s, 4H), 7.80-7.69 (m, 2H), 7.39 (d, J=8.4 Hz, 1H), 7.34-7.21 (m, 1H), 4.18 (s, 3H), 2.97-2.88 (m, 4H), 1.85 (s, 3H), 1.91-1.78 (m, 1H), 1.69

### Example 13: 2-(5-Fluoro-2-((4-(piperidin-1-yl)-3-(1-(222-trifluoroethyl)-1H-indazole-3-carboxamido)phenyl)sulfonamido)phenyl)acetic acid

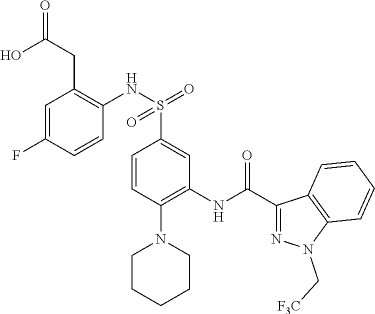 [SMILES: O=C(O)Cc1cc(F)ccc1NS(=O)(=O)c1ccc(N2CCCCC2)c(NC(=O)c2nn(CC(F)(F)F)c3ccccc23)c1]

### Step 1: 1-(4-bromo-2-nitro-phenyl)piperidine

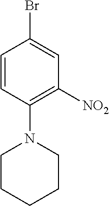 [SMILES: O=[N+]([O-])c1cc(Br)ccc1N1CCCCC1]

To a solution of 4-bromo-1-fluoro-2-nitro-benzene (9.8 g, 44.55 mmol, 5.47 mL, 1 eq) in MeCN (30 mL) was added DIPEA (11.51 g, 89.09 mmol, 15.52 mL, 2 eq) followed by piperidine (4.93 g, 57.91 mmol, 5.72 mL, 1.3 eq). The mixture was stirred at 25° C. for 1 h. The mixture was concentrated under vacuum. The residue was purified by flash silica gel chromatography (80 g SepaFlash® Silica Flash Column, Eluent of 0-10% Ethyl acetate/Petroleum ether gradient @100 mL/min) to afford the title compound (12 g, 42.09 mmol, 94.47% yield) as brown oil.

¹H NMR (400 MHz, CDCl₃) δ=7.91-7.87 (m, 1H), 7.55-7.49 (m, 1H), 6.99 (dd, J=8.8 Hz, 1H), 3.05-2.95 (m, 4H), 1.75-7.67 (m, 4H), 1.64-1.58 (m, 2H).

### Step 2: 1-(4-((4-methoxybenzyl)thio)-2-nitrophenyl)piperidine

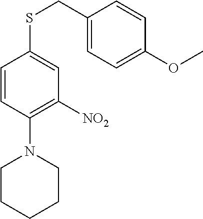 [SMILES: COc1ccc(CSc2ccc(N3CCCCC3)c([N+](=O)[O-])c2)cc1]

To a solution of 1-(4-bromo-2-nitro-phenyl)piperidine (0.5 g, 1.75 mmol, 1 eq) and (4-methoxyphenyl)methanethiol (297.49 mg, 1.93 mmol, 1.1 eq) in dioxane (5 mL) were added Pd₂(dba)₃ (80.29 mg, 87.68 μmol, 0.05 eq), DIPEA (453.27 mg, 3.51 mmol, 2 eq) and Xantphos (50.73 mg, 87.68 μmol, 0.05 eq) under N₂. The mixture was stirred at 100° C. for 16 hr under N₂.

The mixture was concentrated under vacuum. The residue was purified by flash silica gel chromatography (20 g SepaFlash® Silica Flash Column, Eluent of 0˜10% Ethyl acetate/Petroleum ether gradient @60 mL/min) to afford the title compound (0.6 g, 1.67 mmol, 95.46% yield) as brown oil.

¹H NMR (400 MHz, CDCl₃) δ=7.69 (d, J=2.2 Hz, 1H), 7.32 (dd, J=2.3, 8.6 Hz, 1H), 7.19-7.12 (m, 2H), 6.97 (d, J=8.6 Hz, 1H), 6.82 (d, J=8.6 Hz, 2H), 4.00 (s, 2H), 3.80 (s, 3H), 3.04-2.96 (m, 4H), 1.74-1.67 (m, 4H), 1.64-1.57 (m, 2H).

LCMS-AA; MS (ESIpos): m/z=358.9 [M+H]⁺.

### Step 3: 3-nitro-4-(piperidin-1-yl)benzenesulfonyl chloride

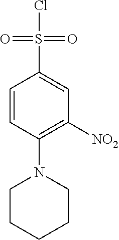 [SMILES: O=[N+]([O-])c1cc(S(=O)(=O)Cl)ccc1N1CCCCC1]

To a solution of 1-(4-((4-methoxybenzyl)thio)-2-nitrophenyl)piperidine (600.00 mg, 1.67 mmol, 1 eq) in DCM (7 mL), AcOH (1 mL) and H₂O (2 mL) was added a solution of 1,3-dichloro-5,5-dimethyl-imidazolidine-2,4-dione (989.34 mg, 5.02 mmol, 3 eq) in DCM (4 mL) slowly at 0° C. The mixture was stirred at 0° C. for 1 h. LCMS-A (with piperidine) showed a peak (48%) with desired mass. The mixture was poured into ice-water (50 mL). The aqueous phase was extracted with DCM (30 mL×2). The combined organic phase was washed with water (20 mL), dried with anhydrous Na₂SO₄, filtered, and concentrated in vacuum to afford the title compound (1 g, crude) as yellow solid.

LCMS-A; MS (ESIpos): m/z=354.2 [M−Cl+piperidine]⁺.

### Step 4: tert-butyl 2-(S-fluoro-2-((3-nitro-4-(piperidin-1-yl)phenyl)sulfonamido)phenyl)acetate

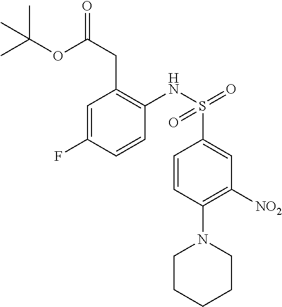 [SMILES: CC(C)(C)OC(=O)Cc1cc(F)ccc1NS(=O)(=O)c1ccc(N2CCCCC2)c([N+](=O)[O-])c1]

A mixture of 3-nitro-4-(piperidin-1-yl)benzenesulfonyl chloride (1 g, crude) and tert-butyl 2-(2-amino-5-fluoro-phenyl)acetate (591.33 mg, 2.63 mmol, 0.8 eq) in Py (5 mL) and DCM (5 mL) was stirred at 25° C. for 2 hr under N₂ atmosphere. The mixture was concentrated in reduced pressure. The residue was poured into water (30 mL). The aqueous phase was extracted with ethyl acetate (30 mL×3). The combined organic phase was washed with brine (30 mL), dried with anhydrous Na₂SO₄, filtered and concentrated in vacuum. The residue was purified by flash silica gel chromatography (40 g SepaFlash® Silica Flash Column, Eluent of 10-30% Ethyl acetate/Petroleum ether gradient @60 mL/min) to give the title compound (0.5 g, 749.68 μmol, 22.85% yield, 74% purity) as brown oil.

¹H NMR (400 MHz, CDCl₃) δ=8.18 (d, J=2.3 Hz, 1H), 8.10 (s, 1H), 7.68 (dd, J=2.3, 9.0 Hz, 1H), 7.34 (dd, J=5.2, 8.9 Hz, 1H), 7.05 (d, J=8.9 Hz, 1H), 6.95 (dt, J=3.0, 8.3 Hz, 1H), 6.89 (dd, J=2.9, 8.7 Hz, 1H), 3.30 (s, 2H), 3.19-3.13 (m, 4H), 1.73-1.60 (m, 6H), 1.45 (s, 9H).

LCMS-AA; MS (ESIpos): m/z=494.1 [M+H]⁺.

### Step 5: tert-butyl 2-(2-((3-amino-4-(piperidin-1-yl)phenyl)sulfonamido)-S-fluorophenyl)acetate

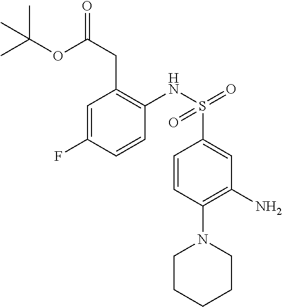 [SMILES: CC(C)(C)OC(=O)Cc1cc(F)ccc1NS(=O)(=O)c1ccc(N2CCCCC2)c(N)c1]

To a solution of tert-butyl 2-(5-fluoro-2-((3-nitro-4-(piperidin-1-yl)phenyl)sulfonamido)phenyl)acetate (0.2 g, 405.23 μmol, 1 eq) in EtOAc (10 mL) was added Pd/C (50 mg, 10% purity) under N₂. The mixture was stirred at 25° C. for 1 hr under H₂ (15 psi). The mixture was filtered and the filtrate was concentrated under vacuum to afford the title compound (0.2 g, 314.95 μmol, 77.72% yield, 73% purity) as brown oil.

LCMS-B; MS (ESIpos): m/z=464.3 [M+H]⁺.

### Step 6: ten-butyl 2-(5-fluoro-2-((4-(piperidin-1-yl)-3-(1-(2,2,2-trifluoroethyl)-1H-indazole-3-carboxamido)phenyl)sulfonamido)phenyl)acetate

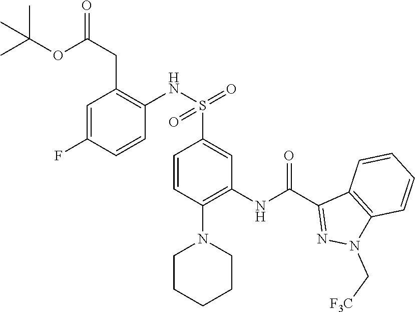 [SMILES: CC(C)(C)OC(=O)Cc1cc(F)ccc1NS(=O)(=O)c1ccc(N2CCCCC2)c(NC(=O)c2nn(CC(F)(F)F)c3ccccc23)c1]

To a solution of tert-butyl 2-(2-((3-amino-4-(piperidin-1-yl)phenyl)sulfonamido)-5-fluorophenyl)acetate (0.2 g, 431.44 μmol, 1 eq), 1-(2,2,2-trifluoroethyl)indazole-3-carboxylic acid (158.02 mg, 647.16 μmol, 1.5 eq) and DIPEA (167.28 mg, 1.29 mmol, 3 eq) in DCM (5 mL) was added HATU (246.07 mg, 647.16 μmol, 1.5 eq). The mixture was stirred at 25° C. for 16 h. The mixture was concentrated under vacuum. The residue was purified by flash silica gel chromatography (20 g SepaFlash® Silica Flash Column, Eluent of 10˜30% Ethylacetate/Petroleum ether gradient @40 mL/min) to afford the title compound (0.18 g, 156.59 μmol, 36.29% yield, 60% purity) as brown oil.

LCMS-T; MS (ESIpos): m/z=690.3 [M+H]⁺.

### Step 7. 2-(5-Fluoro-2-((4-(piperidin-1-yl)-3-(1-(2,2,2-trifluormethyl)-1H-indazole-3-carboxamido)phenyl)sulfonamido)phenyl)acetic acid

To a solution of tert-butyl 2-(5-fluoro-2-((4-(piperidin-1-yl)-3-(1-(2,2,2-trifluoroethyl)-1H-indazole-3-carboxamido)phenyl)sulfonamido)phenyl)acetate (0.18 g, 156.59 μmol, 60/o purity, 1 eq) in DCM (2 mL) was added TFA (3.08 g, 27.01 mmol, 2 mL, 172.51 eq). The mixture was stirred at 25° C. for 0.5 h. The mixture was concentrated under vacuum. The residue was purified by prep-HPLC (column: Phenomenex Synergi C18 150*25 mm*I0 um; mobile phase: [water (FA)-ACN]; B %: 60%-90%, 10 min) followed by lyophilization to afford example 20 (44.5 mg, 68.13 μmol, 43.51% yield, 97% purity) as white solid.

¹H NMR (400 MHz, DMSO-d₆) δ=12.57-12.04 (m, 1H), 9.96 (s, 1H), 8.82 (d, J=2.0 Hz, 1H), 8.29 (d, J=8.2 Hz, 1H), 8.00 (d, J=8.6 Hz, 1H), 7.65-7.60 (m, 1H), 7.44 (t, J=7.6 Hz, 1H), 7.39-7.30 (m, 2H), 7.12 (dd, J=3.1, 9.7 Hz, 1H), 6.98 (dt, J=3.1, 8.5 Hz, 1H), 6.79 (dd, J=5.6, 8.8 Hz, 1H), 5.65 (q, J=9.1 Hz, 2H), 3.61 (s, 2H), 2.93-2.84 (m, 4H), 1.86-1.75 (m, 4H), 1.65-1.56 (m, 2H).

LCMS-B; HPLC-BH; MS (ESIpos): m/z=634.2 [M+H]⁺.

### Example 14: 2-(2-(4-(azetidin-1-yl)-3-(1-(2,2,2-trifluoroethyl)-1H-indazole-3-carboxamido)benzamido)-5-fluorophenyl)acetic acid

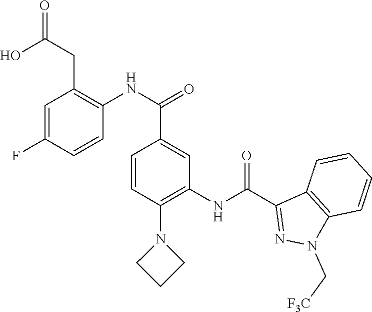 [SMILES: O=C(O)Cc1cc(F)ccc1NC(=O)c1ccc(N2CCC2)c(NC(=O)c2nn(CC(F)(F)F)c3ccccc23)c1]

A mixture of intermediate 21a (150 mg, 239.77 μmol, 1 eq) and TFA (1.93 g, 16.88 mmol, 1.25 mL, 70.41 eq) in DCM (10 mL) was stirred at 25° C. for 0.5 hr. LCMS-B showed one main peak with desired mass was detected. The reaction mixture was concentrated under reduced pressure to give a residue. The residue was purified by prep-HPLC (column: Phenomenex Synergi C18 150*25 mm*10 um; mobile phase: [water(FA)-ACN]; B %: 47%-67%, 10 min) followed by lyophilization to afford the title compound (47 mg, 80.34 μmol, 33.51% yield, 97.347% purity) as a white solid.

¹H NMR (400 MHz, DMSO-d₆) δ=9.82 (br s, 1H), 9.74 (s, 1H), 8.24 (d, J=8.2 Hz, 1H), 7.97-7.89 (m, 2H), 7.79 (dd, J=2.0, 8.6 Hz, 1H), 7.62-7.56 (m, 1H), 7.43-7.35 (m, 2H), 7.18 (dd, J=3.0, 9.7 Hz, 1H), 7.12 (dt, J=3.0, 8.5 Hz, 1H), 6.58 (d, 1=8.6 Hz, 1H), 5.62 (q, J=8.9 Hz, 2H), 4.00 (t, J=7.3 Hz, 4H), 3.63 (s, 2H), 2.23 (q, =7.3 Hz, 2H).

LCMS-H; HPLC-Q; MS (ESIpos): m/z=570.3 [M+H]⁺.

The following examples were prepared in a methodology analogous to the Example 14 with suitable reagents, precursors and starting materials:

| Compound | | | |
|---|---|---|---|
| # | Name | Structure | Analytical Data |
| 40 | 2-(2-(4- (4,4- difluoropiperidin- 1-yl)- 3-(1-(2,2,2- trifluoroethyl)- 1H- indazole-3- carboxamido) benzamido)- 5- fluorophenyl) acetic acid | 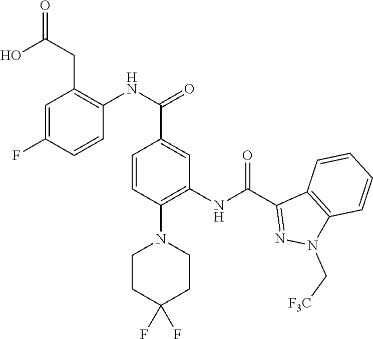 [SMILES: O=C(O)Cc1cc(F)ccc1NC(=O)c1ccc(N2CCC(F)(F)CC2)c(NC(=O)c2nn(CC(F)(F)F)c3ccccc23)c1] | ¹H NMR (400 MHz, DMSO-d₆) δ = 10.30 (br s, 1H), 9.99 (s, 1H), 8.96 (d, J = 1.8 Hz, 1H), 8.32 (d, J = 8.2 Hz, 1H), 7.99 (d, J = 8.6 Hz, 1H), 7.75 (dd, ) = 1.7, 8.3 Hz, 1H), 7.63 (t, J = 7.5 Hz, 1H), 7.50 − 7.41 (m, 3H), 7.23 − 7.11 (m, 2H), 5.66 (q, J = 9.1 Hz, 2H), 3.64 (s, 2H), 3.09 (t, J = 5.1 Hz, 4H), 2.33 − 2.22 (m, 4H). LCMS-AI; HPLC-E; MS (ESIpos): m/z = 634.0 [M + H]⁺. |
| 48 | 2-(2-(4-(4- cyanopiperidin- 1-yl)-3- (1-(2,2,2- trifluoroethyl)- 1H- indazole-3- carboxamido) benzamido)-5- fluorophenyyl) acetic acid | 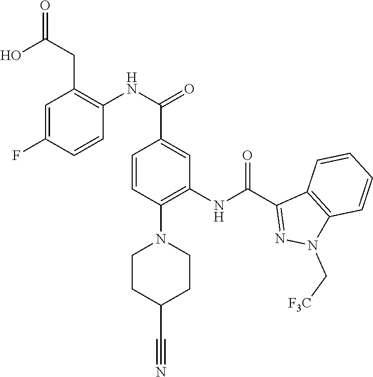 [SMILES: N#CC1CCN(c2ccc(C(=O)Nc3ccc(F)cc3CC(=O)O)cc2NC(=O)c2nn(CC(F)(F)F)c3ccccc23)CC1] | ¹H NMR (400 MHz, DMSO-d₆) δ = 10.12 (br d, J = 1.1 Hz, 1H), 9.98 (s, 1H), 8.96 (d, J = 1.8 Hz, 1H), 8.31 (d, J = 8.2 Hz, 1H), 8.00 (d, J = 8.6 Hz, 1H), 7.74 (dd, J = 1.8, 8.3 Hz, 1H), 7.63 (t, J = 7.7 Hz, 1H), 7.47 − 7.38 (m, 3H), 7.21 (dd, J = 2.9, 9.6 Hz, 1H), 7.16 (dt, J = 2.9, 8.5 Hz, 1H), 5.68 (q, J = 8.9 Hz, 2H), 3.66 (s, 2H), 3.12 (br s, 1H), 3.08 − 3.02 (m, 2H), 2.95 − 2.88 (m, 2H), 2.20 − 2.13 (m, 2H), 2.07 − 1.98 (m, 2H). LCMS-H; HPLC-E; MS (ESIpos): m/z = 623.2 [M + H]⁺. |
| 49 | 2-(2-(4-(2- azaspiro[3.3 heptan-2- yl)-3-(1- (2,2,2- trifluoroethyl)- 1H- indazole-3- carboxamido) benzamido)-5- fluorophenyl) acetic acid | 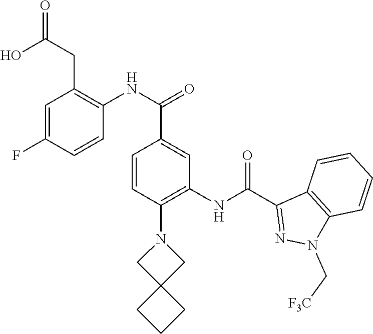 [SMILES: O=C(O)Cc1cc(F)ccc1NC(=O)c1ccc(N2CC3(CCC3)C2)c(NC(=O)c2nn(CC(F)(F)F)c3ccccc23)c1] | ¹H NMR (400 MHz, DMSO-d₆) δ = 12.40 (br s, 1H), 9.91 (br s, 1H), 9.68 (s, 1H), 8.24 (d, J = 8.1 Hz, 1H), 7.99 − 7.94 (m, 2H), 7.78 (dd, J = 2.0, 8.6 Hz, 1H), 7.64 − 7.57 (m, 1H), 7.45 − 7.36 (m, 2H), 7.17 (dd, J = 2.9, 9.7 Hz, 1H), 7.15 − 7.07 (m, 1H), 6.58 (d, J = 8.6 Hz, 1H), 5.64 (q, J = 9.0 Hz, 2H), 3.98 (s, 4H), 3.61 (s, 2H), 2.11 (t, J = 7.6 Hz, 4H), 1.73 (q, J = 7.5 Hz, 2H). LCMS-AO, HPLC-E; MS (ESIpos): m/z = 610.2 [M + H]⁺. |
| 50 | 2-(5-fluoro- 2-(4- (pyrrolidin- 1-yl)-3-(1- (2,2,2- trifluoroethyl)- 1H- indazole-3- carboxamido) benzamido) phenyl) acetic acid | 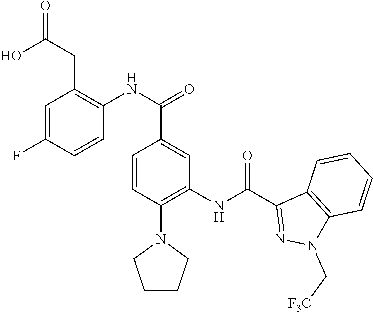 [SMILES: O=C(O)Cc1cc(F)ccc1NC(=O)c1ccc(N2CCCC2)c(NC(=O)c2nn(CC(F)(F)F)c3ccccc23)c1] | ¹H NMR (400 MHz, CDCl₃) δ = 9.25 (s, 1H), 9.14 (br s, 1H), 8.60 (d, J = 1.9 Hz, 1H), 8.33 (d, J = 8.0 Hz, 1H), 7.84 (dd, J = 2.1, 8.5 Hz, 1H), 7.68 − 7.58 (m, 1H), 7.55 − 7.44 (m, 2H), 7.32 (t, J = 7.4 Hz, 1H), 7.06 (d, J = 8.6 Hz, 1H), 7.01 − 6.92 (m, 2H), 5.00 (q, J = 8.3 Hz, 2H), 3.63 (s, 2H), 3.30 − 3.23 (m, 4H), 2.06 − 1.94 (m, 4H). LCMS-H, HPLC-Q; MS (ESIpos): m/z = 584.3 [M + H]⁺. |
| 52 | 2-(2-(4-(3- azabicyclo [3.1.0]hexan- 3-yl)-3-(1- (2,2,2- trifluoroethyl)- 1H- indazole-3- carboxamido) benzamido)-5- fluorophenyl) acetic acid | 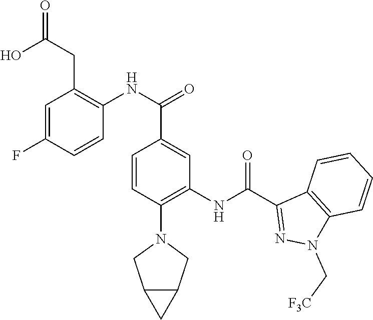 [SMILES: O=C(O)Cc1cc(F)ccc1NC(=O)c1ccc(N2CC3CC3C2)c(NC(=O)c2nn(CC(F)(F)F)c3ccccc23)c1] | ¹H NMR (400 MHz, DMSO-d₆) δ = 9.94 (d, J = 1.3 Hz, 1H), 9.85 (s, 1H), 8.39 (d, J = 2.0 Hz, 1H), 8.27 (d, J = 8.1 Hz, 1H), 7.97 (d, J = 8.6 Hz, 1H), 7.74 (dd, J = 2.1, 8.6 Hz, 1H), 7.61 (dt, J = 1.0, 7.8 Hz, 1H), 7.48 − 7.35 (m, 2H), 7.24 − 7.08 (m, 3H), 5.61 (q, J = 9.0 Hz, 2H), 3.64 (s, 2H), 3.49 (d, J = 9.3 Hz, 2H), 3.23 (d, J = 8.8 Hz, 2H), 1.64 − 1.56 (m, 2H), 0.75 (q, J = 3.7 Hz, 1H), 0.60 (dt, J = 4.4, 7.6 Hz, 1H). LCMS-H; HPLC-E; MS (ESIpos): m/z = 596.3 [M + H]⁺. |
| 53 | 2-(2-(4-(5- azaspiro[2.4] heptan-5- yl)-3-(1- (2,2,2- trifluoroethyl)- 1H- indazole-3- carboxamido) benzamido)-5- fluorophenyl) acetic acid | 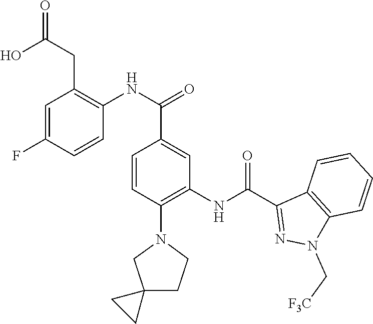 [SMILES: O=C(O)Cc1cc(F)ccc1NC(=O)c1ccc(N2CCC3(CC3)C2)c(NC(=O)c2nn(CC(F)(F)F)c3ccccc23)c1] | ¹H NMR (400 MHz, DMSO-d₆) δ = 10.01 (br s, 1H), 9.91 (s, 1H), 8.24 (d, J = 8.1 Hz, 1H), 8.08 (d, J = 2.1 Hz, 1H), 7.95 (d, J = 8.6 Hz, 1H), 7.80 (dd, J = 2.2, 8.7 Hz, 1H), 7.59 (ddd, J = 1.0, 7.1, 8.4 Hz, 1H), 7.46 − 7.36 (m, 2H), 7.18 (dd, J = 2.9, 9.7 Hz, 1H), 7.12 (dt, J = 3.1, 8.6 Hz, 1H), 6.92 (d, J = 8.7 Hz, 1H), 5.61 (q, J = 9.0 Hz, 2H), 3.62 (s, 2H), 3.54 (t, J = 6.7 Hz, 2H), 3.32 (s, 2H), 1.83 (t, J = 6.7 Hz, 2H), 0.59 − 0.51 (m, 4H). LCMS-Y; HPLC-E; MS (ESIpos): m/z = 610.3 [M + H]⁺. |
| 54 | 2-(2-(4- ((1s,4s)-7- azabicyclo [2.2.1]heptan- 7-yl)-3- (1-(2,2,2- trifluoroethyl)- 1H- indazole-3- carboxamido) benzamido)-5- fluorophenyl) acetic acid | 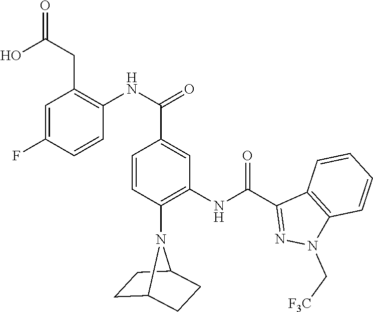 [SMILES: O=C(O)Cc1cc(F)ccc1NC(=O)c1ccc(N2C3CCC2CC3)c(NC(=O)c2nn(CC(F)(F)F)c3ccccc23)c1] | ¹H NMR (400 MHz, DMSO-d₆) δ = 10.02 (br s, 1H), 9.45 (s, 1H), 8.79 (d, J = 2.0 Hz, 1H), 8.32 (d, J = 8.2 Hz, 1H), 7.98 (d, J = 8.6 Hz, 1H), 7.68 (dd, J = 2.0, 8.4 Hz, 1H), 7.65 − 7.59 (m, 1H), 7.48 − 7.38 (m, 2H), 7.23 − 7.11 (m, 3H), 5.65 (q, J = 9.0 Hz, 2H), 4.14 (s, 2H), 3.65 (s, 2H), 1.90 (d, J = 6.8 Hz, 4H), 1.50 (d, J = 6.8 Hz, 4H). LCMS-H; HPLC-E; MS (ESIpos): m/z = 610.3 [M + H]⁺. |
| 56 | 2-(5-fluoro- 2-(4- morpholino- 3-(1- (2,2,2- trifluoroethyl)- 1H- indazole-3- carboxamido) benzamido) phenyl) acetic acid | 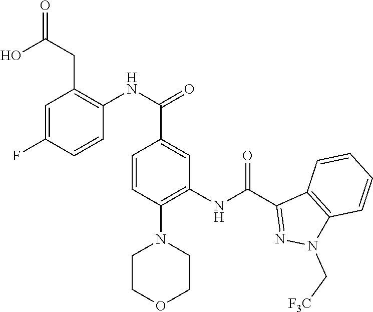 [SMILES: O=C(O)Cc1cc(F)ccc1NC(=O)c1ccc(N2CCOCC2)c(NC(=O)c2nn(CC(F)(F)F)c3ccccc23)c1] | ¹H NMR (400 MHz, DMSO-d₆) δ = 12.54 − 12.31 (m, 1H), 10.08 (s, 1H), 10.03 (s, 1H), 8.99 (d, J = 1.3 Hz, 1H), 8.32 (d, J = 8.1 Hz, 1H), 8.00 (d, J = 8.7 Hz, 1H), 7.76 (d, J = 8.3 Hz, 1H), 7.62 (t, J = 7.7 Hz, 1H), 7.49 − 7.40 (m, 3H), 7.26 − 7.12 (m, 2H), 5.71 (q, J = 9.1 Hz, 2H), 3.94 − 3.85(m, 4H), 3.67 (s, 2H), 3.00-2.92 (m, 4H). LCMS-J; HPLC-P; MS (ESIpos): m/z = 600.1 [M + H]⁺. |
| 57 | 2-(5-fluoro- 2-(4-(4- methylpiperazin- 1-yl)- 3-(1-(2,2,2- trifluoroethyl)- 1H- indazole-3- carboxamido) benzamido) phenyl) acetic acid | 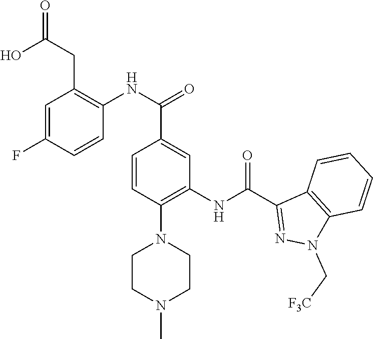 [SMILES: CN1CCN(c2ccc(C(=O)Nc3ccc(F)cc3CC(=O)O)cc2NC(=O)c2nn(CC(F)(F)F)c3ccccc23)CC1] | ¹H NMR (400 MHz, DMSO-d₆) δ = 10.66 − 10.39 (m, 1H), 9.98 (s, 1H), 8.98 (d, J = 1.5 Hz, 1H), 8.31 (d, J = 8.1 Hz, 1H), 8.00 (d, J = 8.4 Hz, 1H), 7.77 (d, J = 8.2 Hz, 1H), 7.62 (t, J = 7.7 Hz, 1H), 7.54 − 7.35 (m, 3H), 7.26 − 7.10 (m, 2H), 5.66 (q, J = 8.9 Hz, 2H), 3.63 (s, 2H), 3.02 − 2.92 (m, 4H), 2.75 − 2.61 (m, 4H), 2.33 (s, 3H). LCMS-J; HPLC-Q; MS (ESIpos): m/z = 613.2 [M + H]⁺. |
| 58 | 2-(2-(4-(2- azabicyclo [2.2.1]heptan- 2-yl)-3- (1-(2,2,2- trifluoroethyl)- 1H- indazole-3- carboxamido) benzamido)-5- fluorophenyl) acetic acid | 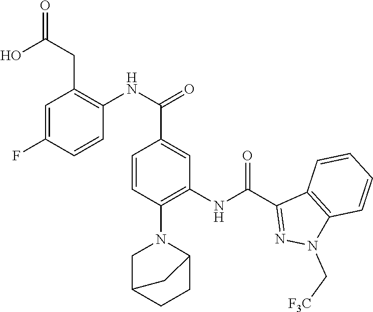 [SMILES: O=C(O)Cc1cc(F)ccc1NC(=O)c1ccc(N2CC3CCC2C3)c(NC(=O)c2nn(CC(F)(F)F)c3ccccc23)c1] | ¹H NMR (400 MHz, DMSO-d₆) δ = 9.83 (s, 1H), 9.73 (s, 1H), 8.25 (d, J = 8.1 Hz, 1H), 8.02 (d, J = 2.1 Hz, 1H), 7.95 (d, J = 8.6 Hz, 1H), 7.76 (dd, J = 2.2, 8.7 Hz, 1H), 7.64 − 7.55 (m, 1H), 7.43 − 7.34 (m, 2H), 7.24 − 7.09 (m, 2H), 6.89 (d, J = 8.8 Hz, 1H), 5.62 (q, J = 9.0 Hz, 2H), 4.23 (s, 1H), 3.65 (s, 2H), 3.60 (d, J = 7.6 Hz, 1H), 2.95 (d, J = 8.8 Hz, 1H), 2.49 − 2.48 (m, 1H), 1.79 (d, J = 10.5 Hz, 1H), 1.70 − 1.56 (m, 3H), 1.44 (d, J = 9.2 Hz, 1H), 1.37 − 1.26 (m, 1H). LCMS-A; HPLC-E; MS (ESIpos): m/z = 610.2 [M + H]⁺. |
| 59 | 2-(5-fluoro- 2-(4-(2- oxopiperidin- 1-yl)-3- (1-(2,2,2- trifluoroethyl)- 1H- indazole-3- carboxamido) benzamido) phenyl) acetic acid | 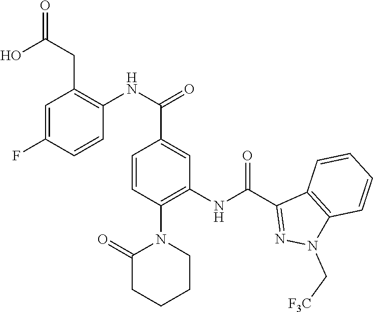 [SMILES: O=C(O)Cc1cc(F)ccc1NC(=O)c1ccc(N2CCCCC2=O)c(NC(=O)c2nn(CC(F)(F)F)c3ccccc23)c1] | ¹H NMR (400 MHz, DMSO-d₆) δ = 12.43 (br s, 1H), 10.10 (s, 1H), 9.22 (s, 1H), 8.73 (d, J = 2.0 Hz, 1H), 8.27 (d, J = 8.1 Hz, 1H), 7.98 (d, J = 8.6 Hz, 1H), 7.82 (dd, J = 2.0, 8.3 Hz, 1H), 7.62 (ddd, J = 1.0, 7.1, 8.4 Hz, 1H), 7.57 (d, J = 8.3 Hz, 1H), 7.43 (dd, J = 6.5, 8.3 Hz, 2H), 7.23 (dd, J = 2.9, 9.7 Hz, 1H), 7.20 − 7.13 (m, 1H), 5.74 − 5.57 (m, 2H), 3.86 − 3.77 (m, 1H), 3.69 (s, 2H), 3.49 − 3.42 (m, 1H), 2.57 − 2.51 (m, 2H), 1.99 − 1.83 (m, 4H). LCMS-AH; HPLC-E; MS (ESIpos): m/z = 612.2 [M + H]⁺. |
| 60 | 2-(5-fluoro- 2-(4-(2- oxooxazolidin- 3-yl)-3- (1-(2,2,2- trifluoroethyl)- 1H- indazole-3- carboxamido) benzamido) phenyl) acetic acid | 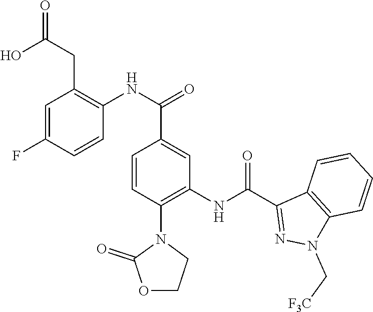 [SMILES: O=C(O)Cc1cc(F)ccc1NC(=O)c1ccc(N2CCOC2=O)c(NC(=O)c2nn(CC(F)(F)F)c3ccccc23)c1] | ¹H NMR (400 MHz, DMSO-d₆) δ = 12.90 − 11.95 (m, 1H), 10.14 (s, 1H), 9.95 (s, 1H), 8.47 (d, J = 1.7 Hz, 1H), 8.24 (d, J = 8.2 Hz, 1H), 7.97 (d, J = 8.4 Hz, 1H), 7.92 − 7.84 (m, 1H), 7.66 (d, J = 8.4 Hz, 1H), 7.61 (t, J = 7.8 Hz, 1H), 7.47 − 7.37 (m, 2H), 7.23 (dd, J = 2.9, 9.6 Hz, 1H), 7.17 (dt, J = 3.1, 8.5 Hz, 1H), 5.64 (q, J = 8.9 Hz, 2H), 4.50 (t, J = 7.8 Hz, 2H), 4.13 (t, J = 7.8 Hz, 2H), 3.67 (s, 2H). LCMS-H; HPLC-E; MS (ESIpos): m/z = 600.2 [M + H]⁺. |
| 61 | 2-(2-(4- (4,4- dimethylpiperidin- 1-yl)- 3-(1-(2,2,2- trifluoroethyl)- 1H- indazole-3- carboxamido) benzamido)-5- fluorophenyl) acetic acid | 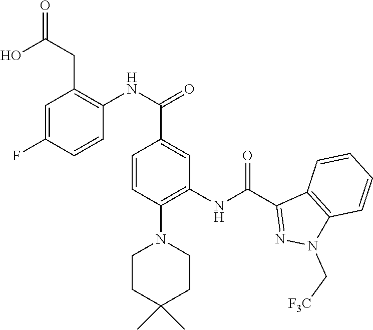 [SMILES: CC1(C)CCN(c2ccc(C(=O)Nc3ccc(F)cc3CC(=O)O)cc2NC(=O)c2nn(CC(F)(F)F)c3ccccc23)CC1] | ¹H NMR (400 MHz, DMSO-d₆) δ = 10.13 − 9.94 (m, 2H), 8.98 (d, J = 2.1 Hz, 1H), 8.33 (d, J = 8.2 Hz, 1H), 8.00 (d, J = 8.6 Hz, 1H), 7.74 (dd, J = 2.1, 8.3 Hz, 1H), 7.69 − 7.57 (m, 1H), 7.51 − 7.38 (m, 3H), 7.28 − 7.11 (m, 2H), 5.64 (q, J = 9.1 Hz, 2H), 3.68 (s, 2H), 2.95 − 2.89 (m, 4H), 1.70 − 1.58 (m, 4H), 1.06 (s, 6H). LCMS-A; HPLC-E; MS (ESIpos): m/z = 626.2 [M + H]⁺. |
| 62 | 2-(2-(4- (3,3- difluoropiperidin- 1-yl)- 3-(1-(2,2,2- trifluoroethyl)- 1H- indazole-3- carboxamido) benzamido)-5- fluorophenyl) acetic acid | 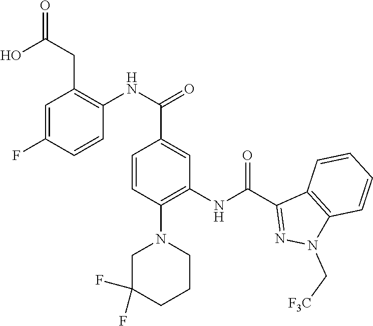 [SMILES: O=C(O)Cc1cc(F)ccc1NC(=O)c1ccc(N2CCCC(F)(F)C2)c(NC(=O)c2nn(CC(F)(F)F)c3ccccc23)c1] | ¹H NMR (400 MHz, DMSO-d₆) δ = 12.42 (br s, 1H), 10.01 (s, 1H), 9.90 (s, 1H), 8.99 (d, J = 2.0 Hz, 1H), 8.31 (d, J = 8.2 Hz, 1H), 7.99 (d, J = 8.6 Hz, 1H), 7.75 (dd, J = 2.1, 8.3 Hz, 1H), 7.62 (ddd, J = 1.0, 7.1, 8.3 Hz, 1H), 7.47 − 7.39 (m, 3H), 7.22 (dd, J = 2.9, 9.7 Hz, 1H), 7.20 − 7.12 (m, 1H), 5.58 (q, J = 9.0 Hz, 2H), 3.67 (s, 2H), 3.29 (br s, 2H), 2.96 − 1.87 (m, 2H), 2.15 − 2.04 (m, 2H), 2.03 − 1.95 (m, 2H). LCMS-AH; HPLC-E; MS (ESIpos): m/z = 634.2 [M + H]⁺. |
| 63 | 2-(2-(4- (1,4- oxazepan-4- yl)-3-(1- (2,2,2- trifluoroethyl)- 1H- indazole-3- carboxamido) benzamido)- 5- fluorophenyl) acetic acid | 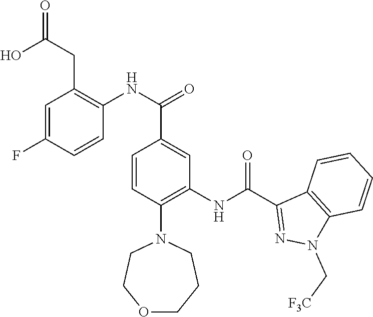 [SMILES: O=C(O)Cc1cc(F)ccc1NC(=O)c1ccc(N2CCCOCC2)c(NC(=O)c2nn(CC(F)(F)F)c3ccccc23)c1] | ¹H NMR (400 MHz, DMSO-d₆) δ = 10.12 (s, 2H), 8.95 (d, J = 2.0 Hz, 1H), 8.33 (d, J = 8.2 Hz, 1H), 8.00 (d, J = 8.6 Hz, 1H), 7.74 (dd, J = 2.0, 8.3 Hz, 1H), 7.63 (t, J = 7.2 Hz, 1H), 7.49 − 7.41 (m, 3H), 7.22 (dd, J = 3.0, 9.7 Hz, 1H), 7.16 (dt, J = 3.0, 8.5 Hz, 1H), 5.67 (q, J = 9.1 Hz, 2H), 3.98 (t, J = 6.2 Hz, 2H), 3.87 − 3.83(m, 2H), 3.67 (s, 2H), 3.20 − 3.14 (m, 4H), 2.12 − 2.05 (m, 2H). LCMS-H; HPLC-E; MS (ESIpos): m/z = 614.3 [M + H]⁺. |
| 64 | 2-(5-fluoro- 2-(4-(4- methylpiperidin- 1-yl)- 3-(1-(2,2,2- trifluoroethyl)- 1H- indazole-3- carboxamido) benzamido) phenyl) acetic acid | 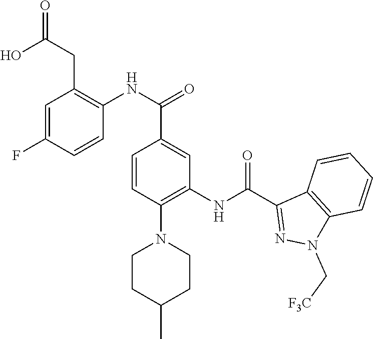 [SMILES: CC1CCN(c2ccc(C(=O)Nc3ccc(F)cc3CC(=O)O)cc2NC(=O)c2nn(CC(F)(F)F)c3ccccc23)CC1] | ¹H NMR (400 MHz, DMSO-d₆) δ = 12.42 (br s, 1H), 10.03 (s, 1H), 9.99 (s, 1H), 8.97 (d, J = 2.0 Hz, 1H), 8.32 (d, J = 8.3 Hz, 1H), 8.00 (d, J = 8.3 Hz, 1H), 7.72 (dd, J = 2.0, 8.3 Hz, 1H), 7.62 (dd, J = 7.0, 8.2 Hz, 1H), 7.47 − 7.40 (m, 2H), 7.37 (d, J = 8.3 Hz, 1H), 7.22 (dd, ) = 2.9, 9.8 Hz, 1H), 7.16 (dt, J = 2.9, 8.6 Hz, 1H), 5.71 − 5.58 (m, 2H), 3.67 (s, 2H), 3.08 − 3.01 (m, 2H), 2.77 (t, J = 10.5 Hz, 2H), 1.78 (d, J = 9.3 Hz, 2H), 1.60 − 1.48 (m, 3H), 1.02 (d, J = 5.4 Hz, 3H). LCMS-H; HPLC-Q; MS (ESIpos): m/z = 612.3 [M + H]⁺. |
| 65 | 2-(5-fluoro- 2-(4-(4- methoxypiperidin- 1-yl)- 3-(1-(2,2,2- trifluoroethyl)- 1H- indazole-3- carboxamido) benzamido) phenyl) acetic acid | 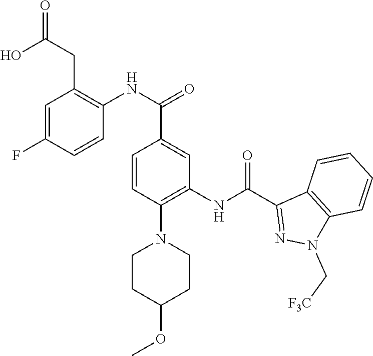 [SMILES: COC1CCN(c2ccc(C(=O)Nc3ccc(F)cc3CC(=O)O)cc2NC(=O)c2nn(CC(F)(F)F)c3ccccc23)CC1] | ¹H NMR (400 MHz, DMSO-d₆) δ = 10.02 − 9.95 (m, 2H), 8.97 (d, J = 2.0 Hz, 1H), 8.32 (d, J = 8.2 Hz, 1H), 7.99 (d, J = 8.6 Hz, 1H), 7.73 (dd, J = 2.0, 8.3 Hz, 1H), 7.66 − 7.59 (m, 1H), 7.50 − 7.34 (m, 3H), 7.29 − 7.10 (m, 2H), 5.64 (q, J = 9.0 Hz, 2H), 3.67 (s, 2H), 3.47 − 3.43 (m, 1H), 3.31 (s, 3H), 3.13 − 3.04 (m, 2H), 2.87 − 2.77 (m, 2H), 2.11 − 2.03 (m, 2H), 1.87 − 1.76 (m, 2H). LCMS-A; HPLC-E; MS (ESIpos): m/z = 628.2 [M + H]⁺. |
| 66 | 2-(2-(4- (1,1- dioxidothio- morpholino)- 3-(1- (2,2,2- trifluoroethyl)- 1H- indazole-3- carboxamido) benzamido)-5- fluorophenyl) acetic acid | 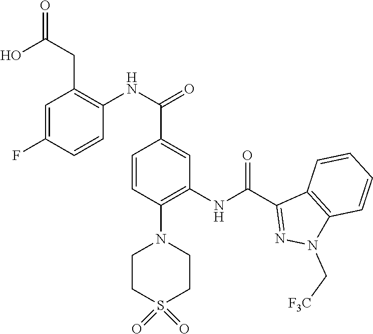 [SMILES: O=C(O)Cc1cc(F)ccc1NC(=O)c1ccc(N2CCS(=O)(=O)CC2)c(NC(=O)c2nn(CC(F)(F)F)c3ccccc23)c1] | ¹H NMR (400 MHz, DMSO-d₆) δ = 13.43 − 11.45 (m, 1H), 10.07 (s, 1H), 10.01 (s, 1H), 8.92 (d, J = 1.8 Hz, 1H), 8.31 (d, J = 8.2 Hz, 1H), 7.99 (d, J = 8.6 Hz, 1H), 7.75 (dd, J = 1.8, 8.3 Hz, 1H), 7.63 (t, J = 7.7 Hz, 1H), 7.53 (d, J = 8.3 Hz, 1H), 7.49 − 7.38 (m, 2H), 7.22 (dd, J = 2.9, 9.7 Hz, 1H), 7.16 (dt, J = 3.0, 8.5 Hz, 1H), 5.68 (q, J = 9.1 Hz, 2H), 3.67 (s, 2H), 3.46 − 3.40 (m, 8H). LCMS-Y; HPLC-E; MS (ESIpos): m/z = 648.1 [M + H]⁺. |
| 67 | 2-(S-fluoro- 2-(4-(2- methylpiperidin- 1-yl)- 3-(1-(2,2,2- trifluoroethyl)- 1H- indazole-3- carboxamido) benzamido) phenyl) acetic acid | 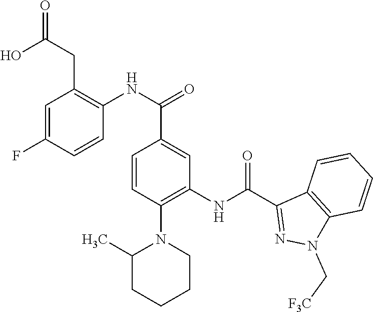 [SMILES: CC1CCCCN1c1ccc(C(=O)Nc2ccc(F)cc2CC(=O)O)cc1NC(=O)c1nn(CC(F)(F)F)c2ccccc12] | ¹H NMR (400 MHz, DMSO-d₆) δ = 13.19 − 11.72 (m, 1H), 10.62 (s, 1H), 10.05 (s, 1H), 9.03 (d, J = 2.1 Hz, IH), 8.31 (d, J = 8.2 Hz, 1H), 8.00 (d, J = 8.6 Hz, 1H), 7.72 (dd, J = 2.0, 8.3 Hz, 1H), 7.62 (dd, J = 7.1, 8.4 Hz, 1H), 7.50 (d, J = 8.2 Hz, 1H), 7.47 − 7.40 (m, 2H), 7.22 (dd, J = 2.9, 9.7 Hz, 1H), 7.16 (dt, J = 3.0, 8.5 Hz, 1H), 5.65 (q, J = 9.0 Hz, 2H), 3.68 (s, 2H), 3.13 − 3.06 (m, 1H), 2.90 (d, J = 11.9 Hz, 1H), 2.77 − 2.68 (m, 1H), 1.95 − 1.88 (m, 1H), 1.87 − 1.69 (m, 3H), 1.55 − 1.44 (m, 2H), 0.82 (d, J = 6.1 Hz, 3H). LCMS-H; HPLC-E; MS (ESIpos): m/z = 612.3 [M + H]⁺. |
| 68 | 2-(2-(4-(2,6- dimethylpiperidin- 1-yl)- 3-(1-(2,2,2- trifluoroethyl)- 1H- indazole-3- carboxamido) benzamido)-5- fluorophenyl) acetic acid | 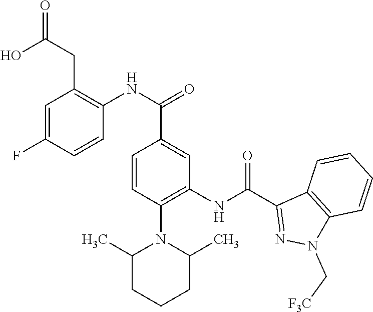 [SMILES: CC1CCCC(C)N1c1ccc(C(=O)Nc2ccc(F)cc2CC(=O)O)cc1NC(=O)c1nn(CC(F)(F)F)c2ccccc12] | ¹H NMR (400 MHz, DMSO-d₆) δ = 12.44 (br s, 1H), 10.97 (s, 1H), 10.06 (s, 1H), 9.04 (s, 1H), 8.31 (d, J = 7.9 Hz, 1H), 8.01 (d, J = 8.6 Hz, 1H), 7.75 (d, J = 7.6 Hz, 1H), 7.67 − 7.56 (m, 2H), 7.43 (t, J = 7.2 Hz, 2H), 7.23 (d, J = 9.4 Hz, 1H), 7.17 (t, J = 8.1 Hz, 1H), 5.65 (q, J = 8.6 Hz, 2H), 3.69 (s, 2H), 3.03 − 2.86 (m, 2H), 1.83 (d, J = 10.3 Hz, 3H), 1.63 − 1.41 (m, 3H), 0.70 (br d, J = 5.6 Hz, 6H). LCMS-H; HPLC-Q; MS (ESIpos): m/z = 626.3 [M + H]⁺. |
| 69 | 2-(5-fluoro- 2-(4-(2- (hydroxymethyl) piperidin- 1-yl)-3- (1-(2,2,2- trifluoroethyl)- 1H- indazole-3- carboxamido) benzamido) phenyl) acetic acid | 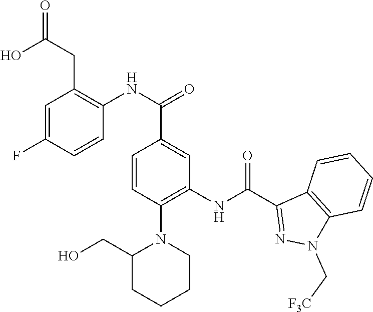 [SMILES: O=C(O)Cc1cc(F)ccc1NC(=O)c1ccc(N2CCCCC2CO)c(NC(=O)c2nn(CC(F)(F)F)c3ccccc23)c1] | ¹H NMR (400 MHz, DMSO-d₆) δ = 13.08 − 11.71 (m, 1H), 10.49 (s, 1H), 10.05 (s, 1H), 9.00 (d, J = 2.0 Hz, 1H), 8.32 (d, J = 8.3 Hz, 1H), 8.00 (d, J = 8.3 Hz, 1H), 7.72 (dd, J = 2.0, 8.3 Hz, 1H), 7.61 (t, J = 7.7 Hz, 1H), 7.52 (d, J = 8.3 Hz, 1H), 7.48 − 7.39 (m, 2H), 7.22 (dd, J = 2.9, 9.3 Hz, 1H), 7.16 (dt, J = 2.9, 8.6 Hz, 1H), 5.69 − 5.58 (m, 2H), 4.41 (t, J = 5.1 Hz, 1H), 3.68 (s, 2H), 3.27 − 3.23 (m, 1H), 3.19 − 3.12 (m, 1H), 3.07 − 3.00 (m, 1H), 2.97 − 2.90 (m, 1H), 2.78 − 2.70 (m, 1H), 2.09 (dd, J = 3.7, 8.1 Hz, 1H), 1.88 − 1.69 (m, 3H), 1.65 − 1.46 (m, 2H). LCMS-H; HPLC-Q; MS (ESIpos): m/z = 628.3 [M + H]⁺. |
| 71 | 2-(2-(4-(2- carbamoylpiperidin- 1- yl)-3-(1- (2,2,2- trifluoroethyl)-1H- indazole-3- carboxamido) benzamido)-5- fluorophenyyl) acetic acid | 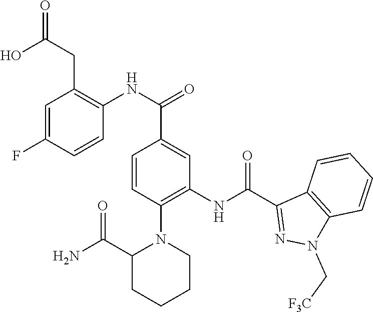 [SMILES: NC(=O)C1CCCCN1c1ccc(C(=O)Nc2ccc(F)cc2CC(=O)O)cc1NC(=O)c1nn(CC(F)(F)F)c2ccccc12] | ¹H NMR (400 MHz, DMSO-d₆) δ = 10.39 (s, 1H), 8.89 (d, J = 1.8 Hz, 1H), 8.33 (d, J = 8.1 Hz, 1H), 7.99 (d, J = 8.7 Hz, 1H), 7.76 (d, J = 7.8 Hz, 1H), 7.62 (t, J = 7.4 Hz, 2H), 7.43 (t, J = 7.6 Hz, 1H), 7.37 (s, 1H), 7.33 (d, J = 8.3 Hz, 1H), 7.18 − 7.05 (m, 2H), 6.77 (s, 1H), 5.77 − 5.55 (m, 2H), 3.75 − 3.68 (m, 1H), 3.55 (s, 2H), 3.47-3.41 (m, 1H), 3.17 − 3.10 (m, 1H), 2.05 − 1.95 (m, 1H), 1.92 − 1.63 (m, 4H), 1.53 − 1.40 (m, 1H). LCMS-N; HPLC-BL; MS (ESIpos): m/z = 641.3 [M + H]⁺. |
| 72 | 2-(2-(4-(2- ethylpiperidin- 1-yl)-3- (1-(2,2,2- trifluoroethyl)- 1H- indazole-3- carboxamido) benzamido)-5- fluorophenyl) acetic acid | 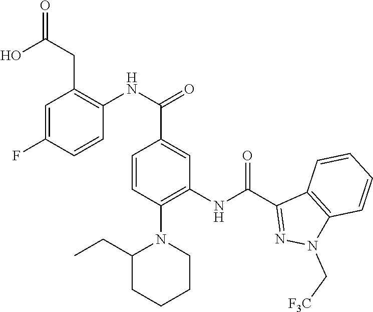 [SMILES: CCC1CCCCN1c1ccc(C(=O)Nc2ccc(F)cc2CC(=O)O)cc1NC(=O)c1nn(CC(F)(F)F)c2ccccc12] | ¹H NMR (400 MHz, DMSO-d₆) δ = 10.63 (s, 1H), 10.12 (s, 1H), 9.03 (d, J = 2.0 Hz, 1H), 8.32 (d, J = 8.2 Hz, 1H), 8.01 (d, J = 8.7 Hz, 1H), 7.72 (dd, J = 2.0, 8.3 Hz, 1H), 7.67 − 7.59 (m, 1H), 7.50 (d, J = 8.3 Hz, 1H), 7.47 − 7.40 (m, 2H), 7.26 − 7.13 (m, 2H), 5.65 (q, J = 8.7 Hz, 2H), 3.68 (s, 2H), 3.02 − 2.88 (m, 2H), 2.72 − 2.66 (m, 1H), 2.01 − 1.68 (m, 4H), 1.60 − 1.43 (m, 2H), 1.34 − 1.17 (m, 2H), 0.68 (t, J = 7.4 Hz, 3H). LCMS-A; HPLC-E; MS (ESIpos): m/z = 626.2 [M + H]⁺. |
| 73 | 2-(5-fluoro- 2-(4-(3- methylpiperidin- 1-yl)- 3-(1-(2,2,2- trifluoroethyl)- 1H- indazole-3- carboxamido) benzamido) phenyl) acetic acid | 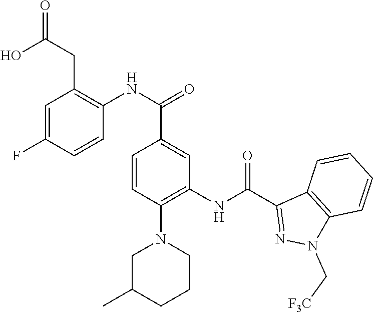 [SMILES: CC1CCCN(c2ccc(C(=O)Nc3ccc(F)cc3CC(=O)O)cc2NC(=O)c2nn(CC(F)(F)F)c3ccccc23)C1] | ¹H NMR (400 MHz, DMSO-d₆) δ = 10.12 − 9.94 (m, 2H), 8.99 (d, J = 2.0 Hz, 1H), 8.33 (d, J = 8.2 Hz, 1H), 8.00 (d, J = 8.7 Hz, 1H), 7.74 (dd, J = 2.1, 8.3 Hz, 1H), 7.68 − 7.58 (m, 1H), 7.48 − 7.40 (m, 2H), 7.37 (d, J = 8.3 Hz, 1H), 7.26 − 7.13 (m, 2H), 5.65 (q, J = 8.7 Hz, 2H), 3.67 (s, 2H), 3.08 − 2.98 (m, 2H), 2.74 − 2.67 (m, 1H), 2.43 (t, J = 10.8 Hz, 1H), 2.07 − 1.94 (m, 1H), 1.91 − 1.78 (m, 3H), 1.19 − 1.03 (m, 1H), 0.93 (d, J = 6.6 Hz, 3H). LCMS-A; HPLC-E; MS (ESIpos): m/z = 612.2 [M + H]⁺. |
| 74 | 2-(2-(4-(3,5- dimethylpiperidin- 1-yl)- 3-(1-(2,2,2- trifluoroethyl)- 1H- indazole-3- carboxamido) benzamido)-5- fluorophenyyl) acetic acid | 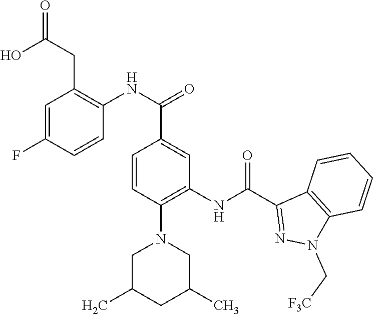 [SMILES: CC1CC(C)CN(c2ccc(C(=O)Nc3ccc(F)cc3CC(=O)O)cc2NC(=O)c2nn(CC(F)(F)F)c3ccccc23)C1] | ¹H NMR (400 MHz, DMSO-d₆) δ = 12.58 − 12.16 (m, 1H), 10.04 − 9.94 (m, 2H), 8.99 (s, 1H), 8.32 (d, J = 8.2 Hz, 1H), 7.98 (d, J = 8.6 Hz, 1H), 7.73 (dd, J = 1.7, 8.3 Hz, 1H), 7.62 (t, J = 7.8 Hz, 1H), 7.47 − 7.39 (m, 2H), 7.35 (d, J = 8.3 Hz, 1H), 7.22 (dd, J = 2.9, 9.7 Hz, 1H), 7.16 (dt, J = 2.9, 8.6 Hz, 1H), 5.63 (q, J = 9.1 Hz, 2H), 3.67 (s, 2H), 3.05 − 2.96 (m, 2H), 2.38 − 1,98 (m, 4H), 1.87 (d, J = 13.0 Hz, 1H), 1.52 − 1.41 (m, 0.5H), 1.07 (br d, J = 6.6 Hz, 1H), 0.88 (d, J = 6.6 Hz, 5H), 0.75 (q, J = 11.8 Hz, 1H). LCMS-Y; HPLC-P; MS (ESIpos): m/z = 626.5 [M + H]⁺. |
| 75 | 2-(5-fluoro- 2-(4-(4- hydroxypiperidin- 1-yl)- 3-(1-(2,2,2- trifluoroethyl)- 1H- indazole-3- carboxamido) benzamido) phenyl) acetic acid | 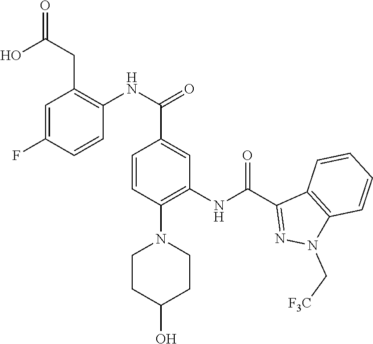 [SMILES: O=C(O)Cc1cc(F)ccc1NC(=O)c1ccc(N2CCC(O)CC2)c(NC(=O)c2nn(CC(F)(F)F)c3ccccc23)c1] | ¹H NMR (400 MHz, DMSO-d₆) δ = 12.42 (br s, 1H), 10.00 (s, 2H), 8.97 (s, 1H), 8.32 (d, J = 8.2 Hz, 1H), 7.99 (d, J = 8.4 Hz, 1H), 7.72 (d, J = 7.5 Hz, 1H), 7.63 (t, J = 7.6 Hz, 1H), 7.48 − 7.35 (m, 3H), 7.25 − 7.12 (m, 2H), 5.73 − 5.59 (m, 2H), 4.76 (d, J = 3.2 Hz, 1H), 3.77 − 3.70 (m, 1H), 3.69 − 3.63 (m, 2H), 3.12 − 3.04 (m, 2H), 2.80 (t, J = 9.2 Hz, 2H), 2.04 − 1.93 (m, 2H), 1.82 − 1.70 (m, 2H). LCMS-H; HPLC-Q; MS (ESIpos): m/z = 614.3 [M + H]⁺. |
| 77 | (R)-2-(5- fluoro-2-(4- (2- methylpiperidin- 1-yl)- 3-(1-(2,2,2- trifluoroethyl)- 1H- indazole-3- carboxamido) benzamido) phenyl) acetic acid | 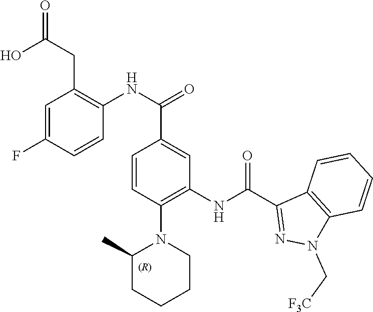 | ¹H NMR (400 MHz, DMSO-d₆) δ = 12.71 − 12.10 (m, 1H), 10.62 (s, 1H), 10.01 (s, 1H), 9.03 (d, J = 2.1 Hz, 1H), 8.32 (d, J = 8.1 Hz, 1H), 8.00 (d, J = 8.6 Hz, 1H), 7.72 (dd, J = 2.0, 8.3 Hz, 1H), 7.62 (t, J = 7.2 Hz, 1H), 7.50 (d, J = 8.2 Hz, 1H), 7.46 − 7.40 (m, 2H), 7.22 (dd, J = 2.9, 9.7 Hz, 1H), 7.16 (dt, J = 2.9, 8.5 Hz, 1H), 5.65 (q, J = 9.1 Hz, 2H), 3.68 (s, 2H), 3.12 − 3.07(m, 1H), 2.90 (d, J = 10.9 Hz, 1H), 2.71 (d, J = 11.0 Hz, 1H), 1.91 (d, J = 7.5 Hz, 1H), 1.87 − 1.78 (m, 2H), 1.76 − 1.73 (m, 1H), 1.55 − 1.46 (m, 2H), 0.82 (d, J = 6.1 Hz, 3H). LCMS-H; HPLC-E; MS (ESIpos): m/z = 612.3 [M + H]⁺. |
| 78 | (S)-2-(5- fluoro-2-(4- (2- methylpiperidin- 1-yl)- 3-(1-(2,2,2- trifluoroethyl)- 1H- indazole-3- carboxamido) benzamido) phenyl) acetic acid | 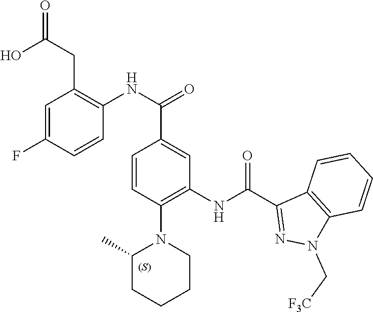 | ¹H NMR (400 MHz, DMSO-d₆) δ = 12.42 (br s, 1H), 10.62 (s, 1H), 10.01 (s, 1H), 9.03 (d, J = 2.0 Hz, 1H), 8.31 (d, J = 8.2 Hz, 1H), 8.00 (d, J = 8.6 Hz, 1H), 7.72 (dd, J = 2.0, 8.2 Hz, 1H), 7.62 (t, J = 7.7 Hz, 1H), 7.50 (d, J = 8.3 Hz, 1H), 7.47 − 7.39 (m, 2H), 7.22 (dd, J = 2.9, 9.7 Hz, IH), 7.16 (dt, J = 3.0, 8.5 Hz, 1H), 5.65 (q, J = 9.0 Hz, 2H), 3.68 (s, 2H), 3.13 − 3.04 (m, 1H), 2.90 (d, J = 11.1 Hz, 1H), 2.74 − 2.67 (m, 1H), 1.95 − 1.88 (m, 1H), 1.87 − 1.77 (m, 2H), 1.76 − 1.70 (m, 1H), 1.56 − 1.44 (m, 2H), 0.82 (d, J = 6.1 Hz, 3H). LCMS-L; HPLC-H; MS (ESIpos): m/z = 612.4 [M + H]⁺. |
| 79 | (R)-2-(5- fluoro-2-(4- (3- methylpiperidin- 1-yl)- 3-(1-(2,2,2- trifluoroethyl)- 1H- indazole-3- carboxamido) benzamido) phenyl) acetic acid | 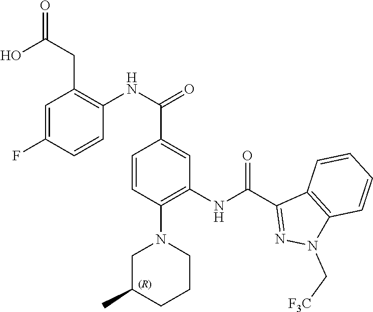 | ¹H NMR (400 MHz, DMSO-d₆) δ = 12.41 (br s, 1H), 9.98 (d, J = 9.0 Hz, 2H), 8.99 (d, J = 2.1 Hz, 1H), 8.33 (d, J = 8.2 Hz, 1H), 7.99 (d, J = 8.6 Hz, 1H), 7.73 (dd, J = 2.0, 8.3 Hz, 1H), 7.62 (t, J = 7.3 Hz, 1H), 7.47 − 7.39 (m, 2H), 7.37 (d, J = 8.3 Hz, 1H), 7.22 (dd, J = 2.9, 9.7 Hz, 1H), 7.16 (dt, J = 3.1, 8.6 Hz, 1H), 5.71 − 5.58 (m, 2H), 3.67 (s, 2H), 3.07 − 2.97 (m, 2H), 2.73 − 2.65 (m, 1H), 2.45 − 2.40 (m, 1H), 2.06 − 1.95 (m, 1H), 1.89 − 1.77 (m, 3H), 1.10 (d, J = 10.8 Hz, 1H), 0.93 (d, J = 6.6 Hz, 3H). LCMS-H; HPLC-E; MS (ESIpos): m/z = 612.4 [M + H]⁺. |
| 80 | (S)-2-(5- fluoro-2-(4- (3- methylpiperidin- 1-yl)- 3-(1-(2,2,2- trifluoroethyl)- 1H- indazole-3- carboxamido) benzamido) phenyl) acetic acid | 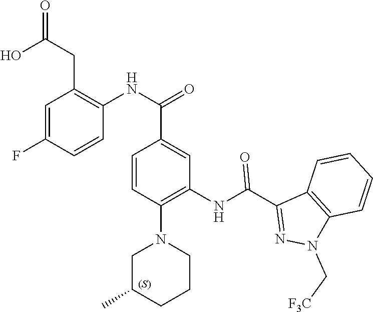 | ¹H NMR (400 MHz, DMSO-d₆) δ = 12.42 (br s, 1H), 9.99 (d, J = 5.3 Hz, 2H), 8.98 (d, J = 2.0 Hz, 1H), 8.32 (d, J = 8.2 Hz, 1H), 7.99 (d, J = 8.6 Hz, 1H), 7.73 (dd, J = 2.0, 8.3 Hz, 1H), 7.62 (t, J = 7.3 Hz, 1H), 7.46 − 7.39 (m, 2H), 7.37 (d, J = 8.3 Hz, 1H), 7.22 (dd, J = 2.9, 9.7 Hz, 1H), 7.16 (dt, J = 3.0, 8.5 Hz, 1H), 5.65 (q, J = 8.9 Hz, 2H), 3.67 (s, 2H), 3.06 − 2.98 (m, 2H), 2.73 − 2.66 (m, 1H), 2.46 − 2.40 (m, 1H), 2.05 − 1.99 (m, 1H), 1.90 − 1.79 (m, 3H), 1.16 − 1.05 (m, 1H), 0.93 (d, J = 6.6 Hz, 3H). LCMS-L; HPLC-H; MS (ESIpos): m/z = 612.4 [M + H]⁺. |
| 82 | 2-(2-(4-(3- (difluoromethyl) piperidin-1-yl)-3- (1-(2,2,2- trifluoroethyl)- 1H- indazole-3- carboxamido) benzamido)-5- fluorophenyl) acetic acid | 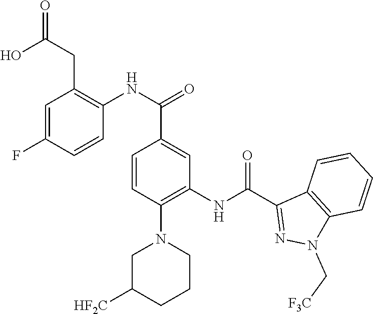 | ¹H NMR (400 MHz, DMSO-d₆) δ = 12.41 (br s, 1H), 10.08 − 9.91 (m, 2H), 8.98 (d, J = 2.1 Hz, 1H), 8.32 (d, J = 8.2 Hz, 1H), 7.99 (d, J = 8.6 Hz, 1H), 7.74 (dd, J = 2.0, 8.3 Hz, 1H), 7.63 (t, J = 7.3 Hz, 1H), 7.47 − 7.39 (m, 3H), 7.22 (dd, J = 2.9, 9.7 Hz, 1H), 7.16 (dt, J = 3.0, 8.5 Hz, 1H), 6.29 − 5.86 (m, 1H), 5.66 (q, J = 9.0 Hz, 2H), 3.67 (s, 2H), 3.19 − 3.13 (m, 1H), 3.03 (d, ) = 11.6 Hz, 1H), 2.76 (t, J = 10.8 Hz, 2H), 2.43 − 2.35 (m, 1H), 1.96 − 1.82 (m, 3H), 1.50 − 1.36 (m, 1H). LCMS-L; HPLC-H; MS (ESIpos): m/z = 648.5 [M + H]⁺. |
| 83 | 2-(2-(4-(4- (difluoromethyl) piperidin- 1-yl)-3- (1-(2,2,2- trifluoroethyl)- 1H- indazole-3- carboxamido) benzamido)-5- fluorophenyl) acetic acid | 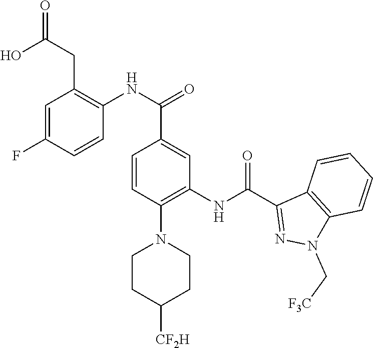 [SMILES: O=C(O)Cc1cc(F)ccc1NC(=O)c1ccc(N2CCC(C(F)F)CC2)c(NC(=O)c2nn(CC(F)(F)F)c3ccccc23)c1] | ¹H NMR (400 MHz, DMSO-d₆) δ = 12.54 − 12.26 (m, 1H), 10.06 (s, 1H), 9.98 (s, 1H), 8.97 (d, J = 1.8 Hz, 1H), 8.32 (d, J = 8.2 Hz, 1H), 7.98 (d, J = 8.6 Hz, 1H), 7.73 (dd, J = 1.8, 8.2 Hz, 1H), 7.63 (t, J = 7.8 Hz, 1H), 7.47 − 7.37 (m, 3H), 7.22 (dd, J = 2.9, 9.6 Hz, IH), 7.16 (dt, J = 2.9, 8.5 Hz, 1H), 6.05 (dt, J = 3.9, 56.8 Hz, 1H), 5.62 (q, J = 9.1 Hz, 2H), 3.67 (s, 2H), 3.18 − 3.12 (m, 2H), 2.87 − 2.78 (m, 2H), 2.10 − 2.00 (m, 1H), 1.90 − 1.76 (m, 4H). LCMS-L; HPLC-J; MS (ESIpos): m/z = 648.5 [M + H]⁺. |
| 84 | 2-(5-fluoro- 2-(4-(3- methoxypiperidin- 1-yl)- 3-(1-(2,2,2- trifluoroethyl)- 1H- indazole-3- carboxamido) benzamido) phenyl) acetic acid | 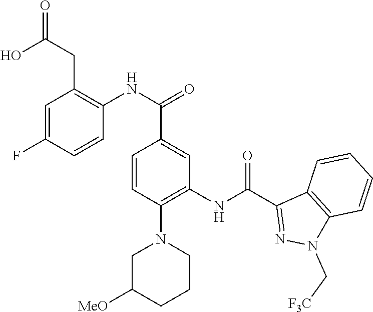 [SMILES: COC1CCCN(c2ccc(C(=O)Nc3ccc(F)cc3CC(=O)O)cc2NC(=O)c2nn(CC(F)(F)F)c3ccccc23)C1] | ¹H NMR (400 MHz, DMSO-d₆) δ = 12.42 (br s, 1H), 9.98 (s, 1H), 9.93 (s, 1H), 8.96 (d, J = 1.8 Hz, 1H), 8.32 (d, J = 8.2 Hz, 1H), 7.99 (d, J = 8.6 Hz, 1H), 7.74 (dd, J = 2.0, 8.3 Hz, 1H), 7.63 (t, J = 7.7 Hz, 1H), 7.46 − 7.37 (m, 3H), 7.22 (dd, J = 2.9, 9.7 Hz, 1H), 7.16 (dt, J = 2.9, 8.5 Hz, 1H), 5.67 (q, J = 9.1 Hz, 2H), 3.67 (s, 2H), 3.58 − 3.51 (m, 1H), 3.30 − 3.28 (m, 1H), 3.23 (s, 3H), 2.97 (d, J = 11.5 Hz, 1H), 2.74 − 2.66 (m, 1H), 2.60 (t, J = 10.0 Hz, 1H), 2.16 − 2.07 (m, 1H), 1.92 − 1.77 (m, 2H), 1.38 − 1.27 (m, 1H). LCMS-L; HPLC-H; MS (ESIpos): m/z = 628.5 [M + H]⁺. |
| 85 | 2-(5-fluoro- 2-(4-(3- hydroxypiperidin- 1-yl)- 3-(1-(2,2,2- trifluoroethyl)- 1H- indazole-3- carboxamido) benzamido) phenyl) acetic acid | 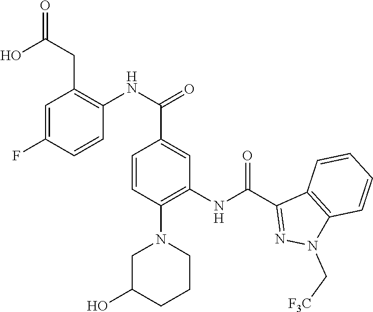 [SMILES: O=C(O)Cc1cc(F)ccc1NC(=O)c1ccc(N2CCCC(O)C2)c(NC(=O)c2nn(CC(F)(F)F)c3ccccc23)c1] | ¹H NMR (400 MHz, DMSO-d₆) δ = 12.89 − 11.86 (m, 1H), 9.96 (d, J = 5.0 Hz, 2H), 8.96 (d, J = 2.0 Hz, 1H), 8.32 (d, J = 8.2 Hz, 1H), 7.99 (d, J = 8.6 Hz, 1H), 7.73 (dd, J = 2.1, 8.3 Hz, 1H), 7.62 (t, J = 7.8 Hz, 1H), 7.46 − 7.36 (m, 3H), 7.22 (dd, J = 2.9, 9.7 Hz, 1H), 7.16 (dt, J =3.1, 8.6 Hz, 1H), 5.70 − 5.60 (m, 2H), 4.87 (br s, 1H), 3.88 − 3.80 (m, 1H), 3.67 (s, 2H), 3.11 (dd, J = 3.7, 10.4 Hz, 1H), 2.96 (d, J = 11.9 Hz, 1H), 2.67 − 2.53 (m, 2H), 2.01 (dd, J = 2.4, 12.4 Hz, 1H), 1.90 − 1.76 (m, 2H), 1.36 − 1.23 (m, 1H). LCMS-L; HPLC-J; MS (ESIpos): m/z = 614.4 [M + H]⁺. |
| 86 | 2-(5-fluoro- 2-(4-(3- (hydroxymethyl) piperidin- 1-yl)-3- (1-(2,2,2- trifluoroethyl)- 1H- indazole-3- carboxamido) benzamido) phenyl) acetic acid | 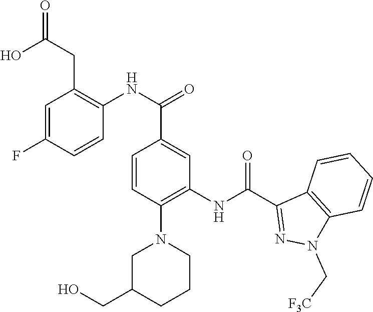 [SMILES: O=C(O)Cc1cc(F)ccc1NC(=O)c1ccc(N2CCCC(CO)C2)c(NC(=O)c2nn(CC(F)(F)F)c3ccccc23)c1] | ¹H NMR (400 MHz, DMSO-d₆) δ = 12.41 (br s, 1H), 10.01 (s, 1H), 9.98 (s, 1H), 8.98 (d, J = 2.0 Hz, 1H), 8.32 (d, J = 8.1 Hz, 1H), 7.99 (d, J = 8.6 Hz, 1H), 7.73 (dd, J = 1.9, 8.3 Hz, 1H), 7.62 (t, J = 7.7 Hz, 1H), 7.46 − 7.36 (m, 3H), 7.22 (dd, J = 2.9, 9.7 Hz, 1H), 7.16 (dt, J = 3.1, 8.5 Hz, 1H), 5.71 − 5.60 (m, 2H), 4.79 − 4.20 (m, 1H), 3.67 (s, 2H), 3.46 − 3.38 (m, 2H), 3.16 (d, J = 8.9 Hz, 1H), 3.01 (d, J = 11.0 Hz, 1H), 2.66 − 2.53 (m, 2H), 2.03 − 1.97 (m, 1H), 1.87 − 1.77 (m, 3H), 1.22 − 1.11 (m, 1H). LCMS-L; HPLC-H; MS (ESIpos): m/z = 628.4 [M + H]⁺. |
| 88 | 2-(2-(4-(3- carbamoylpiperidin- 1- yl)-3-(1- (2,2,2- trifluoroethyl)- 1H- indazole-3- carboxamido) benzamido)-5- fluorophenyl) acetic acid | 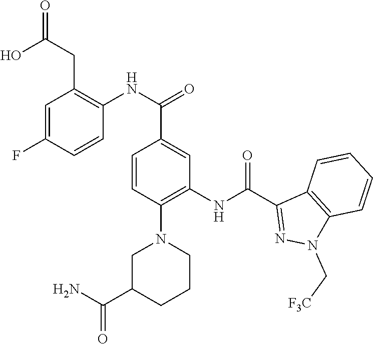 [SMILES: NC(=O)C1CCCN(c2ccc(C(=O)Nc3ccc(F)cc3CC(=O)O)cc2NC(=O)c2nn(CC(F)(F)F)c3ccccc23)C1] | ¹H NMR (400 MHz, DMSO-d₆) δ = 12.40 (br s, 1H), 10.04 (br s, 1H), 9.99 (s, 1H), 8.99 (d, J = 2.1 Hz, 1H), 8.32 (d, J = 8.3 Hz, 1H), 7.99 (d, J = 8.5 Hz, 1H), 7.74 (dd, J = 2.0, 8.4 Hz, 1H), 7.66 − 7.58 (m, 1H), 7.52 − 7.37 (m, 3H), 7.33 (br s, 1H), 7.27 − 7.08 (m, 2H), 6.87 (s, 1H), 5.85 − 5.50 (m, 2H), 3.67 (s, 2H), 3.18 (d, J = 8.8 Hz, 1H), 3.04 (d, J = 11.6 Hz, 1H), 2.93 (t, J = 10.9 Hz, 1H), 2.69 − 2.62 (m, 2H), 1.98 (d, J = 10.8 Hz, 1H), 1.94 − 1.76 (m, 2H), 1.66 − 1.47 (m, 1H). LCMS-H; HPLC-E; MS (ESIpos): m/z = 641.2 [M + H]⁺. |
| 90 | 2-(2-(4-(4- ethylpiperidin- 1-yl)-3- (1-(2,2,2- trifluoroethyl)-1H- indazole-3- carboxamido) benzamido)-5- fluorophenyl) acetic acid | 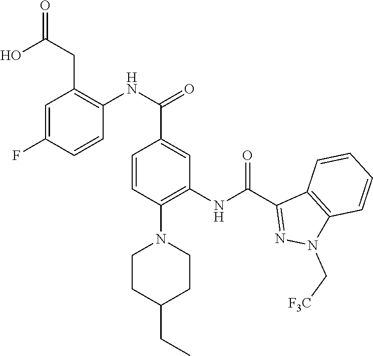 [SMILES: CCC1CCN(c2ccc(C(=O)Nc3ccc(F)cc3CC(=O)O)cc2NC(=O)c2nn(CC(F)(F)F)c3ccccc23)CC1] | ¹H NMR (400 MHz, DMSO-d₆) δ = 12.41 (br s, 1H), 10.01 (s, 1H), 9.97 (s, 1H), 8.97 (d, J = 2.0 Hz, 1H), 8.32 (d, J = 8.2 Hz, 1H), 8.00 (d, J = 8.7 Hz, 1H), 7.72 (dd, J = 2.0, 8.3 Hz, 1H), 7.66 − 7.58 (m, 1H), 7.47 − 7.39 (m, 2H), 7.37 (d, J = 8.3 Hz, 1H), 7.22 (dd, J = 2.9, 9.7 Hz, 1H), 7.16 (dt, J = 2.9, 8.6 Hz, 1H), 5.63 (q, J = 9.0 Hz, 2H), 3.67 (s, 2H), 3.08 (d, J = 11.5 Hz, 2H), 2.76 (t, J = 10.9 Hz, 2H), 1.84 (d, J = 11.9 Hz, 2H), 1.60 − 1.46 (m, 2H), 1.42 − 1.30 (m, 3H), 0.94 (t, J = 7.1 Hz, 3H). LCMS-J; HPLC-BH; MS (ESIpos): m/z = 626.1 [M + H]⁺. |
| 93 | 2-(2-(4-(6- azaspiro[2.5] octan-6- yl)-3-(1- (2,2,2- trifluoroethyl)- 1H- indazole-3- carboxamido) benzamido)- 5- fluorophenyl) acetic acid | 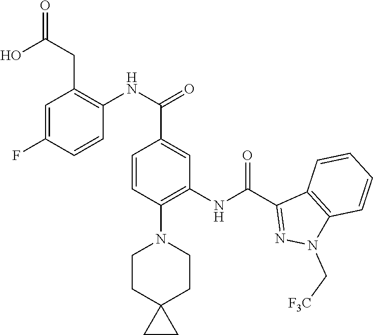 [SMILES: O=C(O)Cc1cc(F)ccc1NC(=O)c1ccc(N2CCC3(CC2)CC3)c(NC(=O)c2nn(CC(F)(F)F)c3ccccc23)c1] | ¹H NMR (400 MHz, DMSO-d₆) δ = 12.40 (br s, 1H), 10.02 (s, 1H), 9.97 (s, 1H), 8.98 (d, J = 2.0 Hz, 1H), 8.32 (d, J = 8.2 Hz, 1H), 8.00 (d, J = 8.6 Hz, 1H), 7.74 (dd, J = 2.0, 8.3 Hz, 1H), 7.62 (t, J = 7.6 Hz, 1H), 7.46 − 7.39 (m, 3H), 7.22 (dd, J = 2.9, 9.7 Hz, 1H), 7.16 (dt, ) = 3.0, 8.5 Hz, 1H), 5.65 (q, J = 9.1 Hz, 2H), 3.67 (s, 2H), 2.97 (t, J = 5.1 Hz, 4H), 1.64 (br s, 4H), 0.42 − 0.36 (m, 4H). LCMS-L; HPLC-H; MS (ESIpos): m/z = 624.5 [M + H]⁺. |
| 153 | (S)-2-(2-(4- (2- ethylpiperidin- 1-yl)-3- (1-(2,2,2- trifluoroethyl)- 1H- indazole-3- carboxamido) benzamido)- 5- fluorophenyl) acetic acid | 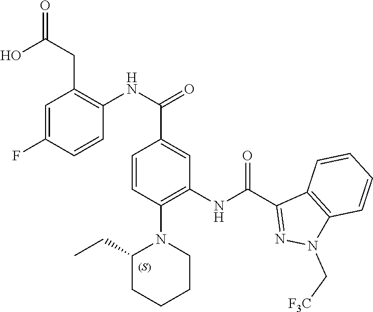 | ¹H NMR (400 MHz, DMSO-d₆) δ = 12.41 (br s, 1H), 10.62 (s, 1H), 10.01 (s, 1H), 9.03 (d, J = 2.0 Hz, 1H), 8.31 (d, J = 8.2 Hz, 1H), 8.00 (d, J = 8.6 Hz, 1H), 7.71 (dd, J = 1.8, 8.2 Hz, 1H), 7.62 (t, J = 7.3 Hz, 1H), 7.50 (d, J = 8.3 Hz, 1H), 7.46 − 7.39 (m, 2H), 7.22 (dd, J = 2.9, 9.7 Hz, 1H), 7.16 (dt, J = 2.9, 8.6 Hz, 1H), 5.70 − 5.59 (m, 2H), 3.68 (s, 2H), 2.98 (s, 1H), 2.91 (d, J = 11.5 Hz, 1H), 2.70 − 2.64 (m, 1H), 1.94 (d, J = 10.0 Hz, 1H), 1.90 − 1.69 (m, 3H), 1.58 − 1.45 (m, 2H), 1.30 − 1.20 (m, 2H), 0.68 (t, J = 7.4 Hz, 3H). LCMS-L; HPLC-H; MS (ESIpos): m/z 626.4 [M + H]⁺. |

### Example 15: 2-(5-fluoro-2-(2-methyl-4-(piperidin-1-yl)-3-(1-(2,2,2-trifluoroethyl)-1H-indazole-3-carboxamido)benzamido)phenyl)acetic acid

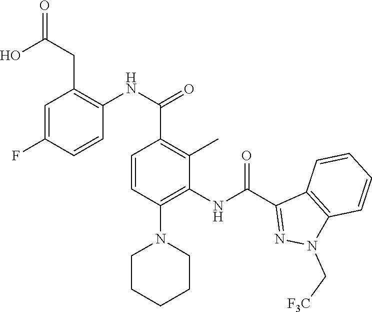 [SMILES: Cc1c(C(=O)Nc2ccc(F)cc2CC(=O)O)ccc(N2CCCCC2)c1NC(=O)c1nn(CC(F)(F)F)c2ccccc12]

### Step 1: 4-chloro-2-methyl-3-nitro-benzoic acid and 4-chloro-2-methyl-5-nitro-benzoic acid

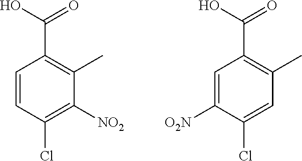

To a solution of HNO₃ (14.52 g, 149.74 mmol, 10.37 mL, 65% purity, 6.16 eq) in H₂SO₄ (20 mL) was added 4-chloro-2-methyl-benzoic acid (4 g, 23.45 mmol, 1 eq) at 0° C. The mixture was stirred at 0° C. for 1 hr. The reaction mixture was poured into cold water 200 mL, the resulting precipitate was filtered, washed with water (100 mL) and dried under reduced pressure to give a mixture (4.4 g, crude) of 4-chloro-2-methyl-3-nitro-benzoic acid and 4-chloro-2-methyl-5-nitro-benzoic acid as a white solid.

LCMS-O; HPLC-Q; MS (ESIpos): m/z=213.9 [M−H]—.

### Step 2: tert-butyl 2-(2-(4-chloro-2-methyl-3-nitrobenzamido)-S-fluorophenyl)acetate and tert-butyl 2-(2-(4-chloro-2-methyl-5-nitrobenzamido)-5-fluorophenyl)acetate

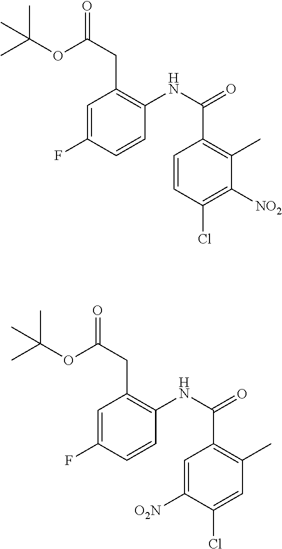 [SMILES: Cc1c(C(=O)Nc2ccc(F)cc2CC(=O)OC(C)(C)C)ccc(Cl)c1[N+](=O)[O-] ; Cc1cc(Cl)c([N+](=O)[O-])cc1C(=O)Nc1ccc(F)cc1CC(=O)OC(C)(C)C]

A mixture of 4-chloro-2-methyl-3-nitro-benzoic acid and 4-chloro-2-methyl-5-nitro-benzoic acid (2 g, crude), tert-butyl 2-(2-amino-5-fluoro-phenyl)acetate (2.09 g, 9.28 mmol, 1 eq), DIPEA (3.60 g, 27.84 mmol, 3 eq) and T₃P (8.86 g, 13.92 mmol, 500% purity, 1.5 eq) in THF (30 mL) was stirred at 25° C. for 1 hr. The reaction mixture was quenched by addition saturated NaHCO₃ solution 60 mL and extracted with ethyl acetate 60 mL. The organic layer was washed with brine 60 mL, dried over Na₂SO₄, filtered, and concentrated under reduced pressure to give a residue. The residue was purified by flash silica gel chromatography (Eluent of 0˜10% Ethyl acetate/Petroleum ether) to give a mixture (3.3 g, crude) tert-butyl 2-(2-(4-chloro-2-methyl-3-nitrobenzamido)-5-fluorophenyl)acetate and tert-butyl 2-(2-(4-chloro-2-methyl-5-nitrobenzamido)-5-fluorophenyl)acetate as a yellow solid.

LCMS-I; MS (ESIpos): m/z=445.2 [M+Na]³⁰.

### Step 3: tert-butyl 2-(5-fluoro-2-(2-methyl-3-nitro-4-(piperidin-1-yl)benzamido)phenyl)acetate and tert-butyl 2-(5-fluoro-2-(2-methyl-5-nitro-4-(piperidin-1-yl)benzamido)phenyl)acetate

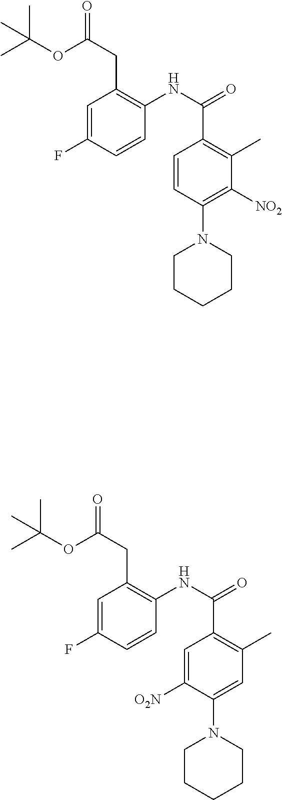 [SMILES: Cc1c(C(=O)Nc2ccc(F)cc2CC(=O)OC(C)(C)C)ccc(N2CCCCC2)c1[N+](=O)[O-] ; Cc1cc(N2CCCCC2)c([N+](=O)[O-])cc1C(=O)Nc1ccc(F)cc1CC(=O)OC(C)(C)C]

A mixture of tert-butyl 2-(2-(4-chloro-2-methyl-3-nitrobenzamido)-5-fluorophenyl)acetate and tert-butyl 2-(2-(4-chloro-2-methyl-5-nitrobenzamido)-5-fluorophenyl)acetate (1.5 g, crude), piperidine (302.27 mg, 3.55 mmol, 1 eq) and DIPEA (916.96 mg, 7.10 mmol, 2 eq) in MeCN (20 mL) was stirred at 80° C. for 48 hr. The reaction mixture was concentrated under reduced pressure to give a residue. The residue was purified by flash silica gel chromatography (Eluent of 0-20% Ethyl acetate/Petroleum ether) to afford a mixture (1.4 g) of tert-butyl 2-(5-fluoro-2-(2-methyl-3-nitro-4-(piperidin-1-yl)benzamido)phenyl)acetate and tert-butyl 2-(5-fluoro-2-(2-methyl-5-nitro-4-(piperidin-1-yl)benzamido)phenyl)acetate as a yellow solid.

LCMS-H; MS (ESIpos): m/z=472.3 [M+H]⁺.

### Step 4: tert-butyl 2-[2-[[3-amino-2-methyl-4-(1-piperidyl)benzoyl)amino]-5-fluoro-phenylacetate and tert-butyl 2-(2-(5-amino-2-methyl-4-(piperidin-1-yl)benzamido)-5-fluorophenyl)acetate

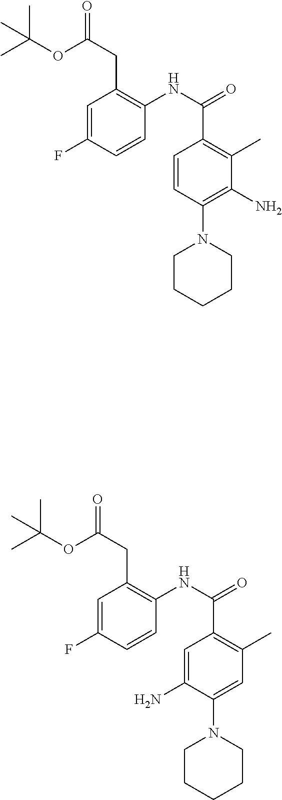 [SMILES: Cc1c(C(=O)Nc2ccc(F)cc2CC(=O)OC(C)(C)C)ccc(N2CCCCC2)c1N ; Cc1cc(N2CCCCC2)c(N)cc1C(=O)Nc1ccc(F)cc1CC(=O)OC(C)(C)C]

A mixture of tert-butyl 2-(5-fluoro-2-(2-methyl-3-nitro-4-(piperidin-1-yl)benzamido)phenyl)acetate and tert-butyl 2-(5-fluoro-2-(2-methyl-5-nitro-4-(piperidin-1-yl)benzamido)phenyl)acetate (1.4 g, crude) and Pd/C (300 mg, 109% purity) in EtOAc (40 mL) was degassed and purged with H₂ for 2 times, and then the mixture was stirred at 25° C. for 1 hr under H₂ atmosphere (15 psi). LCMS-S showed one main peak with desired mass was detected. The reaction mixture was filtered and concentrated under reduced pressure to give a mixture (1.3 g) of tert-butyl 2-[2-[[3-amino-2-methyl-4-(1-piperidyl)benzoyl]amino]-5-fluoro-phenyl]acetate and tert-butyl 2-(2-(5-amino-2-methyl-4-(piperidin-1-yl)benzamido)-5-fluorophenyl)acetate as a white solid.

LCMS-S; MS (ESIpos): m/z=442.1 [M+H]⁺.

### Step 5: tert-butyl 2-(5-fluoro-2-(2-methy-4-(piperidin-1-yl)-3-(1-(2,2,2-trifturoethyl)-1H-indazole-3-carboxamido)benzanido)phenyl)acetate and tert-butyl 2-(5-fluoro-2-(2-methyl-4-(piperidin-1-yl)-S-(1-(2,2,2-trifluoroethyl)-1H-indazole-3-carboxanido)benzamido)phenyl)acetate

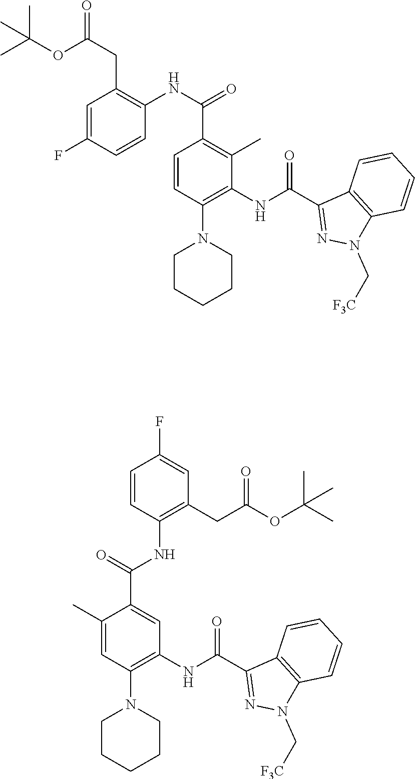 [SMILES: Cc1c(C(=O)Nc2ccc(F)cc2CC(=O)OC(C)(C)C)ccc(N2CCCCC2)c1NC(=O)c1nn(CC(F)(F)F)c2ccccc12 ; Cc1cc(N2CCCCC2)c(NC(=O)c2nn(CC(F)(F)F)c3ccccc23)cc1C(=O)Nc1ccc(F)cc1CC(=O)OC(C)(C)C]

A mixture of tert-butyl 2-[2-[[3-amino-2-methyl-4-(1-piperidyl)benzoyl]amino]-5-fluoro-phenyl]acetate and tert-butyl 2-(2-(5-amino-2-methyl-4-(piperidin-1-yl)benzamido)-5-fluorophenyl)acetate (650 mg, crude), 1-(2,2,2-trifluoroethyl) indazole-3-carboxylic acid (359.45 mg, 1.47 mmol, 1 eq), HATU (839.62 mg, 2.21 mmol, 1.5 eq) and DIPEA (380.52 mg, 2.94 mmol, 2 eq) in DCM (8 mL) was stirred at 25° C. for 15 hr. The reaction mixture was concentrated under reduced pressure. The residue was purified by prep-HPLC (column: Phenomenex Synergi C18 150*25 mm*10 um; mobile phase: [water(FA)-ACN]; B %: 75%-100%, 10 min) followed by lyophilization to afford tert-butyl 2-(5-fluoro-2-(2-methyl-4-(piperidin-1-yl)-3-(1-(2,2,2-trifluoroethyl)-1H-indazole-3-carboxamido)benzamido)phenyl)acetate (210 mg, 311.37 μmol, 21.15% yield, 99% purity) as a white solid.

¹H NMR (400 MHz, CDCl₃) δ=9.01 (s, 1H), 8.85 (s, 1H), 8.48 (d, J=8.2 Hz, 1H), 7.91 (dd, J=5.3, 8.7 Hz, 1H), 7.59-7.49 (m, 3H), 7.43-7.37 (m, 1H), 7.10-6.97 (m, 3H), 5.07 (br d, J=8.2 Hz, 2H), 3.60 (s, 2H), 2.85 (s, 4H), 2.56 (s, 3H), 1.64 (s, 4H), 1.53 (br d, J=4.6 Hz, 2H), 1.44 (s, 9H).

LCMS-S; MS (ESIpos): m/z=668.2 [M+H]⁺.

As a second fraction, tert-butyl 2-(5-fluoro-2-(2-methyl-4-(piperidin-1-yl)-5-(1-(2,2,2-trifluoroethyl)-1H-indazole-3-carboxamido)benzamido)phenyl)acetate (500 mg, 703.92 μmol, 47.82% yield, 94% purity) was isolated as a white solid.

¹H NMR (400 MHz, CDCl₃) δ=9.98 (s, 1H), 9.03-8.77 (m, 2H), 8.49 (d, J=8.1 Hz, 1H), 7.92 (dd, 0.1=5.4, 8.7 Hz, 1H), 7.59-7.47 (m, 2H), 7.44-7.36 (m, 1H), 7.12-6.96 (m, 3H), 5.02 (q, J=8.3 Hz, 2H), 3.65 (s, 2H), 2.92 (s, 4H), 2.55 (s, 3H), 1.88-1.83 (m, 4H), 1.66 (s, 2H), 1.37 (s, 9H).

LCMS-S; MS (ESIpos): m/z=668.2 [M+H]⁺.

### Step 6: 2-(5-fluoro-2-(2-methyl-4-(piperidin-1-yl)-3-(1-(2,2,2-trifluoroethyl)-1H-indazole-3-carboxamido)benzamido)phenyl)acetic acid

A mixture of tert-butyl 2-(5-fluoro-2-(2-methyl-4-(piperidin-1-yl)-3-(1-(2,2,2-trifluoroethyl)-1H-indazole-3-carboxamido)benzamido)phenyl)acetate (100 mg, 149.77 μmol, 1 eq) and TFA (828.42 mg, 7.27 mmol, 48.51 eq) in DCM (10 mL) was stirred at 25° C. for 1 hr. The reaction mixture was concentrated under reduced pressure. The residue was purified by prep-HPLC (column: Phenomenex Synergi C18 150*25 mm*10 um; mobile phase: [water (FA)-ACN]; B %: 51%-81%, 10 min) followed by lyophilization to afford the title compound (69.0 mg, 111.69 μmol, 74.58% yield, 99% purity) as a white solid.

¹H NMR (400 MHz, DMSO-d₆) δ=10.07 (br s, 1H), 9.33 (s, 1H), 8.25 (d, J=8.2 Hz, 1H), 7.96 (d, J=8.6 Hz, 1H), 7.60 (t, J=7.7 Hz, 1H), 7.49 (dd, J=5.7, 8.4 Hz, 1H), 7.46-7.36 (m, 2H), 7.18 (dd, J=2.9, 9.7 Hz, 1H), 7.16-7.09 (m, 1H), 7.05 (d, J=8.2 Hz, 1H), 5.67 (q, J=9.0 Hz, 2H), 3.69 (s, 2H), 2.85-2.75 (m, 4H), 2.30 (s, 3H), 1.55-1.48 (m, 4H), 1.43 (br d, J=4.2 Hz, 2H).

LCMS-H; HPLC-E; MS (ESIpos): m/z=612.3 [M+H]⁺.

### Example 16: 2-(5-fluoro-2-(2-methyl-4-(piperidin-1-yl)-5-(1-(2,2,2-trifluoroethyl)-1H-indazole-3-carboxamido)benzamido)phenyl)acetic acid

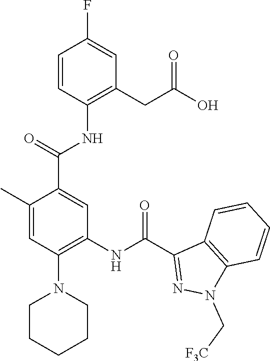 [SMILES: Cc1cc(N2CCCCC2)c(NC(=O)c2nn(CC(F)(F)F)c3ccccc23)cc1C(=O)Nc1ccc(F)cc1CC(=O)O]

A mixture of tert-butyl 2-(5-fluoro-2-(2-methyl-4-(piperidin-1-yl)-5-(1-(2,2,2-trifluoroethyl)-1H-indazole-3-carboxamido)benzamido)phenyl)acetate (from step 5 of example 42) (250 mg, 374.42 μmol, 1 eq) and TFA (3.08 g, 27.01 mmol, 2 mL, 72.14 eq) in DCM (10 mL) was stirred at 25° C. for 1 hr. The reaction mixture was concentrated under reduced pressure. The residue was purified by prep-HPLC (column: Phenomenex Synergi C18 150*25 mm*10 um; mobile phase: [water(FA)-ACN]; B %: 53%-73%, 10 min) followed by lyophilization to afford the title compound (143.2 mg, 231.80 μmol, 61.91% yield, 99% purity) as a white solid.

¹H NMR (400 MHz, DMSO-d₆) δ=10.18 (br s, 1H), 10.01 (s, 1H), 8.59 (s, 1H), 8.31 (d, J=8.2 Hz, 1H), 7.99 (d, j=8.7 Hz, 1H), 7.61 (t, J=7.7 Hz, 1H), 7.53 (br dd, J=5.7, 8.4 Hz, 1H), 7.43 (t, J=7.5 Hz, 1H), 7.22-7.09 (m, 3H), 5.66 (q, J=9.0 Hz, 2H), 3.67 (s, 2H), 2.90-2.82 (m, 4H), 2.39 (s, 3H), 1.85-1.75 (m, 4H), 1.65-1.53 (m, 2H).

LCMS-H; HPLC-E; MS (ESIpos): m/z=612.4 [M+H]⁺.

### Example 17: 2-(5-fluoro-2-(5-(piperidin-1-yl)-4-(1-(2,2,2-trifluoroethyl)-1H-indazole-3-carboxamido)picolinamide)phenyl)acetic acid

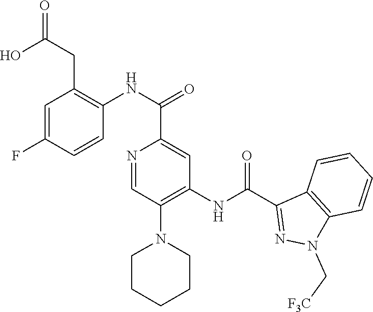 [SMILES: O=C(O)Cc1cc(F)ccc1NC(=O)c1cc(NC(=O)c2nn(CC(F)(F)F)c3ccccc23)c(N2CCCCC2)cn1]

### Step 1: tert-butyl 4-amino-5-(piperidin-1-yl)picolinate

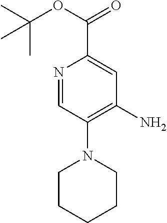 [SMILES: CC(C)(C)OC(=O)c1cc(N)c(N2CCCCC2)cn1]

To an 40 mL vial equipped with a stir bar was added tert-butyl 4-amino-5-bromopicolinate (600 mg, 2.20 mmol, 1 eq), piperidine (1000 mg, 11.74 mmol, 1.16 mL, 5.35 eq), DABCO (246.42 mg, 2.20 mmol, 241.59 uL, 1 eq), Ir[dF(CF₃)ppy]2(dtbpy)(PF₆) (24.65 mg, 21.97 μmol, 0.01 eq) and NiCl₂.dtbbpy (4.37 mg, 10.98 μmol, 0.005 eq) in DMAc (20 mL). The vial was sealed and placed under nitrogen was added. The reaction was stirred and irradiated with a 34 W blue LED lamp (7 cm away), with cooling fan to keep the reaction temperature at 25° C. for 15 h. The reaction mixture was filtered and concentrated under reduced pressure to give a residue. The residue was purified by flash silica gel chromatography (12 g SepaFlash® Silica Flash Column, Eluent of 0˜20% Ethyl acetate/Petroleum ether gradient @40 mL/min) to afford the title compound (210 mg, 719.28 μmol, 32.74% yield, 95% purity) as a white solid.

1H NMR (400 MHz, CDCl₃) δ=8.17 (s, 1H), 7.42 (s, 1H), 4.86 (br s, 2H), 2.93-2.87 (m, 4H), 1.71 (q, J=5.4 Hz, 4H), 1.61 (s, 9H).

LCMS-B; MS (ESIpos): m/z=278.3 [M+H]⁺.

### Step 2: tert-butyl 5-(piperidin-1-yl)-4-(1-(2,2,2-trifluromethyl)-1H-indazole-3-carboxamido)picolinate

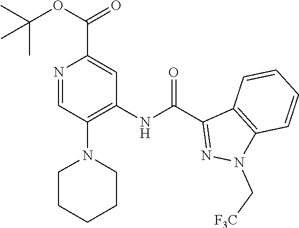 [SMILES: CC(C)(C)OC(=O)c1cc(NC(=O)c2nn(CC(F)(F)F)c3ccccc23)c(N2CCCCC2)cn1]

To a solution of 1-(2,2,2-trifluoroethyl)indazole-3-carboxylic acid (316.92 mg, 1.30 mmol, 2 eq) and DMF (4.74 mg, 64.90 μmol, 0.1 eq) in DCM (6 mL) was added oxalyl chloride (98.85 mg, 778.77 μmol, 1.2 eq) at 0° C. The mixture was stirred at 25° C. for 0.5 h. A solution tert-butyl 4-amino-5-(piperidin-1-yl)picolinate (180 mg, 648.97 μmol, 1 eg) in Py (10 mL) was added. The mixture was stirred at 25° C. for 16 h. The mixture was concentrated under reduced pressure. The residue was poured into saturated NaHCO₃ (20 mL). The aqueous phase was extracted with ethyl acetate (20 mL×3). The combined organic phase was dried with anhydrous Na₂SO₄, filtered, and concentrated in vacuum. The residue was purified by flash silica gel chromatography (20 g SepaFlash® Silica Flash Column, Eluent of 10˜60% Ethyl acetate/Petroleum ether gradient @40 mL/min) to afford the title compound (250 mg, 387.28 μmol, 59.68% yield, 78% purity) as white solid.

LCMS-I; MS (ESIpos): m/z=504.3 [M+H].

### Step 3: 5-(piperidin-1-yl)-4-(1-(2,2,2-trifluoroethyl)-1H-indazole-3-carboxamido)picolinic acid

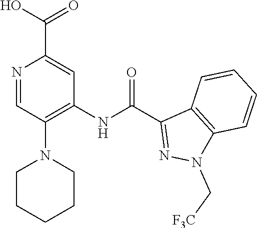 [SMILES: O=C(O)c1cc(NC(=O)c2nn(CC(F)(F)F)c3ccccc23)c(N2CCCCC2)cn1]

A mixture of tert-butyl 5-(piperidin-1-yl)-4-(1-(2,2,2-trifluoroethyl)-1H-indazole-3-carboxamido)picolinate (200.00 mg, 309.82 μmol, 78% purity, 1 eq) and HCl/dioxane (4 M, 10 mL, 129.11 eq) in dioxane (2 mL) was stirred at 25° C. for 16 h. LCMS-A showed a peak (73%, 254 nm) with desired mass. The mixture was concentrated der vacuum to afford the title compound (0.15 g, crude) as white solid.

LCMS-A; MS (ESIpos): m/z=448.1 [M+H]⁺.

### Step 4: tert-butyl 2-(5-fluoro-2-(5-(piperidin-1-yl)-4-(1-(2,2,2-fluoroethyl)-1H-indazole-3-carboxamido)picolinamide)phenyl)acetate

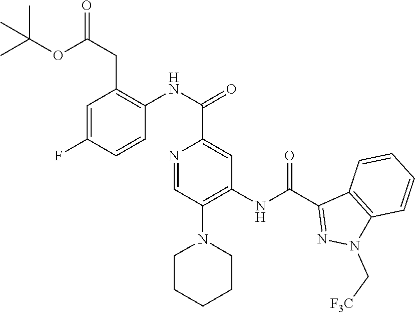 [SMILES: CC(C)(C)OC(=O)Cc1cc(F)ccc1NC(=O)c1cc(NC(=O)c2nn(CC(F)(F)F)c3ccccc23)c(N2CCCCC2)cn1]

To a solution of 5-(piperidin-1-yl)-4-(1-(2,2,2-trifluoroethyl)-1H-indazole-3-carboxamido)picolinic acid (150.00 mg, 335.26 μmol, 1 eq)(crude) and DIPEA (129.99 mg, 1.01 mmol, 3 eq) in DCM (5 mL) was added tert-butyl 2-(2-amino-5-fluoro-phenyl)acetate (90.63 mg, 402.32 μmol, 1.2 eq) followed by HATU (191.22 mg, 502.89 μmol, 1.5 eq). The mixture was stirred at 25° C. for 2 h. The mixture was concentrated under vacuum. The residue was purified by flash silica gel chromatography (20 g SepaFlash® Silica Flash Column, Eluent of 0-15% Ethyl acetate/Petroleum ether gradient (@, 40 mL/min) to afford the title compound (0.1 g, 145.12 μmol, 43.28% yield, 95% purity) as white solid.

¹H NMR (400 MHz, DMSO-d₆) δ=10.25 (s, 1H), 10.07 (s, 1H), 9.13 (s, 1H), 8.47 (s, 1H), 8.32 (d, J=8.3 Hz, 1H), 8.03 (d, J=8.5 Hz, 1H), 7.75 (dd, J=5.5, 8.8 Hz, 1H), 7.68-7.62 (m, 1H), 7.47 (t, J=7.4 Hz, 1H), 7.25-7.17 (m, 2H), 5.69 (q, J=9.1 Hz, 2H), 3.70 (s, 2H), 3.05-2.98 (m, 4H), 1.87-1.80 (m, 4H), 1.69-1.60 (m, 2H), 1.34 (s, 9H).

LCMS-1; MS (ESIpos): m/z=655.2 [M+H]⁺.

### Step 5: 2-(5-fluoro-2-(5-(piperidin-1-yl)-4-(1-(2,2,2-trifluoroethyl)-1H-indazole-3-carboxamido)picolinamide)phenyl)acetic acid

To a solution of tert-butyl 2-(5-fluoro-2-(5-(piperidin-1-yl)-4-(1-(2,2,2-trifluoroethyl)-1H-indazole-3-carboxamido)picolinamido)phenyl)acetate (0.1 g, 152.75 μmol, 1 eq) in DCM (1 mL) was added TFA (1.54 g, 13.51 mmol, 1 mL, 88.42 eq). The mixture was stirred at 25° C. for 1 h. The mixture was concentrated under vacuum. The residue was purified by prep-HPLC (column: Unisil 3-100 C18 Ultra 150*50 mm*3 um; mobile phase: [water(FA)-ACN]; B %: 50%-80%, 10 min) followed by lyophilization to afford the title compound (83.2 mg, 137.61 μmol, 90.09% yield, 99% purity) as white solid.

¹H NMR (400 MHz, DMSO-d₆) δ=10.40 (s, 1H), 10.05 (s, 1H), 9.12 (s, 1H), 8.48 (s, 1H), 8.31 (d, J=8.2 Hz, 1H), 8.02 (d, J=8.6 Hz, 1H), 7.75 (dd, J=5.6, 8.9 Hz, 1H), 7.68-7.61 (m, 1H), 7.47 (t, J=7.6 Hz, 1H), 7.26-7.15 (m, 2H), 5.68 (q, J=9.0 Hz, 2H), 3.69 (s, 2H), 3.05-2.96 (m, 4H), 1.86-1.78 (m, 4H), 1.68-1.58 (m, 2H).

LCMS-AN, HPLC-BH; MS (ESIpos): m/z=599.2 [M+H]⁺

### Example 18: 2-(5-fluoro-2-(6-(piperidin-1-yl)-5-(1-(2,2,2-trifluoroethyl)-1H-indazole-3-carboxamido)nicotinamido)phenyl)acetic acid

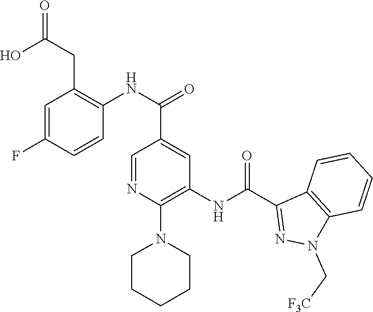 [SMILES: O=C(O)Cc1cc(F)ccc1NC(=O)c1cnc(N2CCCCC2)c(NC(=O)c2nn(CC(F)(F)F)c3ccccc23)c1]

### Step 1: 5-nitro-6-(piperidin-1-yl)nicotinic acid

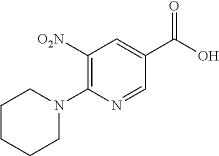 [SMILES: O=C(O)c1cnc(N2CCCCC2)c([N+](=O)[O-])c1]

To a solution of 6-chloro-5-nitronicotinic acid (1 g, 4.94 mmol, 1 eq) in MeCN (10 mL) was added DIPEA (1.28 g, 9.87 mmol, 1.72 mL, 2 eq) followed by piperidine (420.37 mg, 4.94 mmol, 1 eq). The mixture was stirred at 40° C. for 1 h. The mixture was concentrated under vacuum to afford the title compound (1.3 g, crude) as brown solid.

LCMS-J; MS (ESIpos): m/z=252.2 [M+H].

### Step 2: tert-butyl 2-(S-fluoro-2-(5-nitro-6-(piperidin-1-yl)nicotinamido)phenyl)acetate

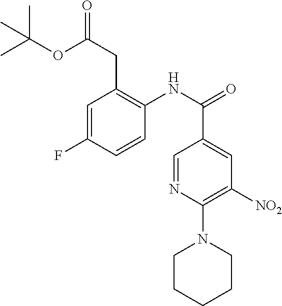 [SMILES: CC(C)(C)OC(=O)Cc1cc(F)ccc1NC(=O)c1cnc(N2CCCCC2)c([N+](=O)[O-])c1]

To a solution of 5-nitro-6-(piperidin-1-yl)nicotinic acid (0.3 g, 1.19 mmol, 1 eq) (crude), tert-butyl 2-(2-amino-5-fluoro-phenyl)acetate (268.98 mg, 1.19 mmol, 1 eq) and DIPEA (308.65 mg, 2.39 mmol, 2 eq) in MeCN (4 mL) was added T₃P (1.14 g, 1.79 mmol, 1.07 mL, 50% purity, 1.5 eq). The mixture was stirred at 25° C. for 6 h. The mixture was concentrated under vacuum. The residue was poured into water (10 mL). The aqueous phase was extracted with ethyl acetate (10 mL×2). The combined organic phase was dried with anhydrous Na₂SO₄, filtered, and concentrated in vacuum. The residue was purified by flash silica gel chromatography (12 g SepaFlash® Silica Flash Column, Eluent of 0-10/6 Ethyl acetate/Petroleum ether gradient @30 mL/min) to afford the title compound (0.4 g, 0.864 mmol, 72.33% yield, 99% purity) as yellow solid.

¹H NMR (400 MHz, CDCl₃) δ=9.89 (br s, 1H), 8.94 (d, J=2.2 Hz, 1H), 8.75 (d, J=2.2 Hz, 1H), 7.97 (dd, J=5.4, 8.9 Hz, 1H), 7.08-7.02 (m, 1H), 6.97 (dd, J=2.9, 8.7 Hz, 1H), 3.58 (s, 2H), 3.56-3.50 (m, 4H), 1.50 (s, 9H), 1.23-1.13 (m, 6H).

LCMS-H; MS (ESIpos): m/z=459.3 [M+H]⁺.

### Step 3: tert-butyl 2-(2-(5-anion-6-(piperidin-1-yl)nicotinamido)-S-fluorophenyl)acetate

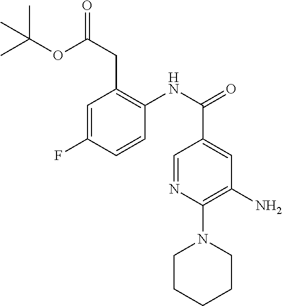 [SMILES: CC(C)(C)OC(=O)Cc1cc(F)ccc1NC(=O)c1cnc(N2CCCCC2)c(N)c1]

To a solution of tert-butyl 2-(5-fluoro-2-(5-nitro-6-(piperidin-1-yl)nicotinamido)phenyl)acetate (0.2 g, 0.436 mmol, 1 eq) in EtOAc (5 mL) was added Pd/C (50 mg, 10% purity) under N₂. The mixture was stirred at 25° C. for 0.5 h under H₂ (15 psi). The mixture was filtered, and the filtrate was concentrated under vacuum to afford the title compound (0.2 g, crude) as white solid.

### Step 4: tert-butyl 2-(5-fluoro-2-(6-(piperidin-1-yl)-5-(1-(2,2,2-trifluoroethyl)-1H-indazole-3-carboxamido)nicotinamido)phenyl)acetate

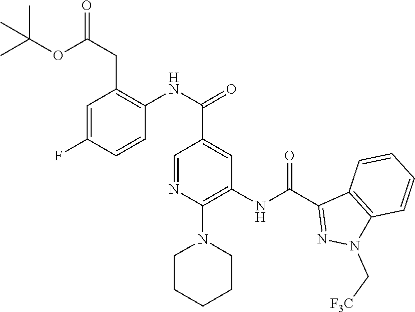 [SMILES: CC(C)(C)OC(=O)Cc1cc(F)ccc1NC(=O)c1cnc(N2CCCCC2)c(NC(=O)c2nn(CC(F)(F)F)c3ccccc23)c1]

To a solution of tert-butyl 2-(2-(5-amino-6-(piperidin-1-yl)nicotinamido)-5-fluorophenyl)acetate (200 mg, 0.468 mmol, 1 eq)(crude), 1-(2,2,2-trifluoroethyl)indazole-3-carboxylic acid (170.95 mg, 0.700 mmol, 1.5 eq) and DIPEA (120.64 mg, 0.933 mmol, 2 eq) in DCM (5 mL) was added HATU (266.21 mg, 0.700 mmol, 1.5 eq). The mixture was stirred at 25° C. for 16 h. The mixture was concentrated under vacuum. The residue was purified by flash silica gel chromatography (12 g SepaFlash® Silica Flash Column, Eluent of 10˜30% Ethyl acetate/Petroleum ether gradient @40 mL/min) to afford the title compound (0.3 g, 0.297 mmol, 63.82% yield, 65% purity) as off-white solid.

LCMS-S; MS (ESIpos): m/z=655.3 [M+H]⁺.

### Step 5: 2-(5-fluoro-2-(6-(piperidin-1-yl)-5-(1-(2,2,2-trifluoroethyl)-1H-indazole-3-carboxamido)nicotinamido)phenyl)acetic acid

To a solution of tert-butyl 2-(5-fluoro-2-(6-(piperidin-1-yl)-5-(1-(2,2,2-trifluoroethyl)-1H-indazole-3-carboxamido)nicotinamido)phenyl)acetate (300.45 mg, 0.298 mmol, 65% purity, 1 eq) in DCM (3 mL) was added TFA (4.62 g, 40.52 mmol, 3.00 mL, 135.82 eq). The mixture was stirred at 25° C. for 1 h. The mixture was concentrated under vacuum. The residue was purified by prep-HPLC (column: Unisil 3-100 C18 Ultra 150*50 mm*3 um; mobile phase: [water(FA)-ACN]; B %: 45%-75%, 10mmin) followed by lyophilization to afford the title compound (157 mg, 0.256 mmol, 85.90% yield, 97.7% purity) as white solid.

¹H NMR (400 MHz, DMSO-d₆) δ=12.44 (br s, 1H), 10.23 (br s, 1H), 9.56 (s, 1H), 8.98 (d, =2.0 Hz, 1H), 8.68 (dd, J=2.1 Hz, 1H), 8.30 (d, J=8.1 Hz, 1H), 8.00 (d, J=8.5 Hz, 1H), 7.63 (t, J=7.4 Hz, 1H), 7.51-7.38 (m, 2H), 7.22 (dd, J=2.9, 9.6 Hz, 1H), 7.19-7.13 (m, 1H), 5.67 (q, J=9.0 Hz, 2H), 3.67 (s, 2H), 3.19-3.12 (m, 4H), 1.83-1.72 (m, 4H), 1.67-1.57 (m, 2H).

LCMS-Y, HPLC-BH; MS (ESIpos): m/z=599.2 [M+H]˜.

### Example 19: 2-(5-fluoro-2-(5-(piperidin-1-yl)-6-(1-(2,2,2-trifluoroethyl)-1H-indazole-3-carboxamido)picolinamido)phenyl)acetic acid

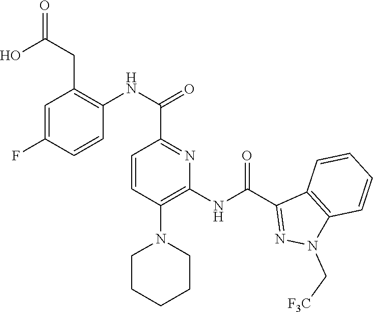 [SMILES: O=C(O)Cc1cc(F)ccc1NC(=O)c1ccc(N2CCCCC2)c(NC(=O)c2nn(CC(F)(F)F)c3ccccc23)n1]

### Step 1: (6-amino-5-(piperidin-1-yl)pyridin-2-yl)(piperidin-1-yl)methanone

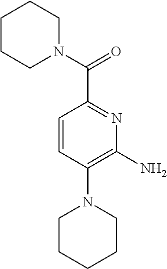 [SMILES: Nc1nc(C(=O)N2CCCCC2)ccc1N1CCCCC1]

To an 40 mL vial equipped with a stir bar was added methyl 6-amino-5-bromopicolinate (1 g, 4.29 mmol, 1 eq), piperidine (1.95 g, 22.96 mmol, 2.27 mL, 5.35 eq), DABCO (481.36 mg, 4.29 mmol, 1 eq), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (48.14 mg, 42.91 μmol, 0.01 eq), NiCl₂.dtbbpy (8.54 mg, 21.46 μmol, 0.005 eq) in DMAc (30 mL). The vial was sealed and placed under nitrogen. The reaction was stirred and irradiated with a 34 W blue LED lamp (7 cm away), with cooling fan to keep the reaction temperature at 25° C. for 15 h. The mixture was diluted with H₂O (50 mL) and extracted with EtOAc (50 mL×2). The combined organic layer was concentrated to afford crude residue. The crude residue was purified by flash silica gel chromatography (12 g SepaFlash® Silica Flash Column, Eluent of 0-60% Ethyl acetate/Petroleum ether gradient @80 mL/min) to afford the title compound (1 g, 3.02 mmol, 70.30% yield, 87% purity) as yellow oil.

LCMS-A; MS (ESIpos): m/z=289.2 [M+H]⁺.

### Step 2: 6-amino-5-(piperidin-1-yl)picolinic acid

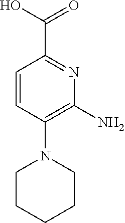 [SMILES: Nc1nc(C(=O)O)ccc1N1CCCCC1]

To a solution of (6-amino-5-(piperidin-1-yl)pyridin-2-yl(piperidin-1-yl)methanone (610 mg, 2.12 mmol, 1 eq) in H₂O (15 mL) and EtOH (30 mL) was added NaOH (3.38 g, 84.61 mmol, 40 eq). The mixture was stirred at 80° C. for 16 h. The mixture was adjusted to a pH of 7 with 1M HCl, then diluted with H₂O (60 mL) and extracted with EtOAc (60 mL×2). The combined organic layer was concentrated to afford crude residue. The crude residue was purified by prep-HPLC (column: Unisil 3-100 C18 Ultra 150*50 mm*3 um; mobile phase: [water(FA)-ACN]; B %: 1%-45%, 10 min). The eluent was concentrated and freeze-dried to afford the title compound (240 mg, 1.06 mmol, 50.26% yield, 98% purity) as yellow oil.

¹H NMR (400 MHz, DMSO-d₆) δ=7.32-7.27 (m, 1H), 7.26-7.20 (m, 1H), 6.43-5.61 (m, 2H), 2.90-2.70 (m, 4H), 1.74-1.64 (m, 4H), 1.60-1.47 (m, 2H).

LCMS-A; MS (ESIpos): m/z=222.2 [M+H]⁺

### Step 3: tert-butyl 2-(2-(6-amino-5-(piperidin-1-yl)picolinamido)-5-fluorophenyl)acetate

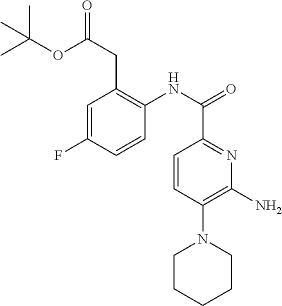 [SMILES: CC(C)(C)OC(=O)Cc1cc(F)ccc1NC(=O)c1ccc(N2CCCCC2)c(N)n1]

To a solution of 6-amino-5-(piperidin-1-yl)picolinic acid (140 mg, 632.75 μmol, 1 eq) and tert-butyl 2-(2-amino-5-fluoro-phenyl)acetate (185.29 mg, 822.58 μmol, 1.3 eq) in DMF (3 mL) were added HATU (360.89 mg, 949.13 μmol, 1.5 eq) and DIPEA (245.33 mg, 1.90 mmol, 3 eq). The mixture was stirred at 25° C. for 1.5 h. The mixture was diluted with H₂O (10 mL) and extracted with EtOAc (10 mL×2). The combined organic layer was concentrated to afford crude residue. The crude residue was purified by flash silica gel chromatography (12 g SepaFlash® Silica Flash Column, Eluent of 0˜50% Ethyl acetate/Petroleum ether gradient @80 mL/min) to afford the title compound (100 mg, 231.04 μmol, 36.51% yield, 99% purity) as a white solid.

¹H NMR (400 MHz, DMSO-d₆) δ=10.00 (s, 1H), 7.82 (dd, J=5.5, 8.8 Hz, 1H), 7.39-7.33 (m, 1H), 7.30-7.25 (m, 1H), 7.23-7.10 (m, 2H), 5.66 (s, 2H), 2.88-2.79 (m, 4H), 1.76-1.65 (m, 4H), 1.55 (d, J=5.0 Hz, 2H), 1.30 (s, 9H).

LCMS-A; MS (ESIpos): m/z=429.2 [M+H]⁺.

### Step 4: tert-butyl 2-(5-fluoro-2-(5-(piperidin-1-yl)-6-(1-(2,2,2-trifluoroethyl)-1H-indazole-3-carboxamido)picolinamido)phenyl)acetate

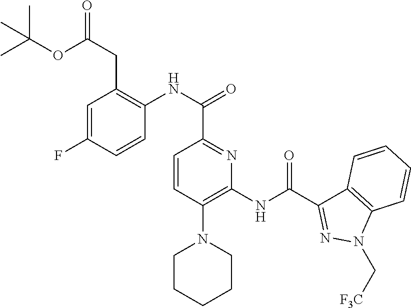 [SMILES: CC(C)(C)OC(=O)Cc1cc(F)ccc1NC(=O)c1ccc(N2CCCCC2)c(NC(=O)c2nn(CC(F)(F)F)c3ccccc23)n1]

To a solution of tert-butyl 2-(2-(6-amino-5-(piperidin-1-yl)picolinamido)-5-fluorophenyl)acetate (40 mg, 93.35 μmol, 1 eq) and 1-(2,2,2-trifluoroethyl)indazole-3-carboxylic acid (45.59 mg, 186.70 μmol, 2 eq) in pyridine (1 mL) was added POCl₃ (71.57 mg, 466.75 μmol, 5 eq). The mixture was stirred at 25° C. for 1 h. The mixture was diluted with H₂O (10 mL) and extracted with EtOAc (10 mL×2). The combined organic layer was concentrated to afford crude residue. The crude residue was purified by flash silica gel chromatography (12 g SepaFlash® Silica Flash Column, Eluent of 0˜50% Ethyl acetate/Petroleum ether gradient @80 mL/min) to afford the title compound (63 mg, 90.46 μmol, 96.91% yield, 94% purity) as a white solid. LCMS-A; MS (ESIpos): m/z=655.2 [M+H]⁺.

### Step 5: 2-(5-fluro-2-(5-(piperidin-1-yl)-6-(1-(2,2,2-fluoroethyl)-1H-indazole-3-carboxamido)picolinamido)phenyl)acetic acid

To a solution of tert-butyl 2-(5-fluoro-2-(5-(piperidin-1-yl)-6-(1-(2,2,2-trifluoroethyl)-1H-indazole-3-carboxamido)picolinamido)phenyl)acetate (63 mg, 96.23 μmol, 1 eq) in dichloromethane (3 mL) was added TFA (4.62 g, 40.52 mmol, 3 mL, 421.04 eq). The mixture was stirred at 25° C. for 1 h. The mixture was concentrated to afford crude residue. The residue was purified by prep-HPLC (column: Unisil 3-100 C18 Ultra 150*50 mm*3 um; mobile phase: [water(FA)-ACN]; B %: 48%-78%, 10 min). The eluent was concentrated and freeze-dried to afford the title compound (29.6 mg, 47.97 μmol, 49.85% yield, 97% purity) as a white solid.

¹H NMR (400 MHz, DMSO-d( ) δ=12.69-12.33 (m, 1H), 10.46 (s, 1H), 10.06 (s, 1H), 8.40 (d, J=8.2 Hz, 1H), 8.16 (dd, J=5.6, 8.9 Hz, 1H), 8.00 (d, J=8.6 Hz, 1H), 7.90 (d, J=8.1 Hz, 1H), 7.78 (d, J=8.1 Hz, 1H), 7.64 (t, J=7.7 Hz, 1H), 7.45 (t, J=7.5 Hz, 1H), 7.29-7.15 (m, 2H), 5.68 (q, J=9.0 Hz, 2H), 3.92 (s, 2H), 3.04-2.91 (m, 4H), 1.85-1.74 (m, 4H), 1.61 (d, J=4.5 Hz, 2H).

LCMS-A; HPLC-E; MS (ESIpos): m/z=599.2 [M+H]⁺.

### Example 20: 2-(2-((4-(piperidin-1-yl)-3-((1-(2,2,2-trifluoroethyl)-1H-indazol-3-yl)carbamoyl)phenyl)carbamoyl)phenyl)acetic acid

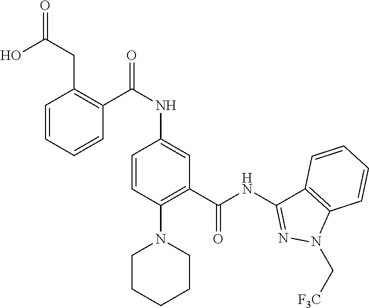 [SMILES: O=C(O)Cc1ccccc1C(=O)Nc1ccc(N2CCCCC2)c(C(=O)Nc2nn(CC(F)(F)F)c3ccccc23)c1]

### Step 1: 5-nitro-2-(piperidin-1-yl)benzoic acid

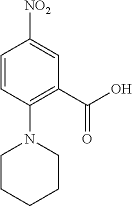 [SMILES: O=C(O)c1cc([N+](=O)[O-])ccc1N1CCCCC1]

To a solution of 2-fluoro-5-nitro-benzoic acid (1 g, 5.40 mmol, 1 eq) in dioxane (20 mL) was added piperidine (2.30 g, 27.01 mmol, 2.67 mL, 5 eq) at 25° C. The mixture was stirred at 25° C. for 16 h. The mixture was concentrated under vacuum. The residue was purified by flash silica gel chromatography (40 g SepaFlash® Silica Flash Column, Eluent of 50-100% Ethyl acetate/Petroleum ether to 10% methanol/Ethyl acetate gradient @60 mL/min) to afford the title compound (0.6 g, 2.37 mmol, 43.94% yield, 99% purity) as yellow solid.

¹H NMR (400 MHz, DMSO-d₆) δ=14.12-13.30 (m, 1H), 8.35 (d, J=2.8 Hz, 1H), 8.16 (dd, J=2.8, 9.3 Hz, 1H), 7.19 (d, 0.1=9.3 Hz, 1H), 3.29-3.20 (m, 4H), 1.67-1.55 (m, 6H).

LCMS-AB; MS (ESIpos): m/z=250.8 [M+H]˜.

### Step 2: 5-nitro-2-(piperidin-1-yl)-N-(1-(2,2,2-trifluoroethyl)-1H-indazol-3-y)benzamide

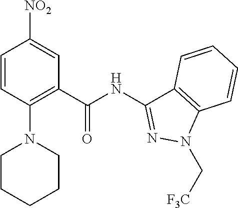 [SMILES: O=C(Nc1nn(CC(F)(F)F)c2ccccc12)c1cc([N+](=O)[O-])ccc1N1CCCCC1]

To a solution of 5-nitro-2-(piperidin-1-yl)benzoic acid (0.3 g, 1.20 mmol, 1 eq) and 1-(2,2,2-trifluoroethyl)indazol-3-amine (257.95 mg, 1.20 mmol, 1 eq) in DMF (6 mL) were added HATU (683.73 mg, 1.80 mmol, 1.5 eq) and DIPEA (309.87 mg, 2.40 mmol, 2 eq). The mixture was stirred at 25° C. for 16 h. The mixture was poured into ice water (100 mL). The aqueous phase was extracted with ethyl acetate (50 mL×3). The combined organic phase was washed with brine (50 mL×3), dried with anhydrous Na₂SO₄, filtered, and concentrated in vacuum. The residue was purified by flash silica gel chromatography (20 g SepaFlash® Silica Flash Column, Eluent of 0-15% Ethyl acetate/Petroleum ether gradient @40 mL/min) to afford the title compound (0.4 g, 563.24 μmol, 46.98% yield, 63% purity) as yellow solid.

¹H NMR (400 MHz, DMSO-d₆) δ=11.27 (s, 1H), 8.35 (d, J=2.7 Hz, 1H), 8.22 (dd, J=2.6, 9.2 Hz, 1H), 7.96-7.90 (m, 1H), 7.78 (d, J=8.6 Hz, 1H), 7.53-7.46 (m, 1H), 7.25-7.19 (m, 2H), 5.41 (q, J=9.0 Hz, 2H), 3.32-3.26 (m, 4H), 1.68-1.53 (m, 6H).

LCMS-AI; MS (ESIpos): m/z=447.9 [M+H]⁺.

### Step 3: 5-amino-2-(piperidin-1-yl)-N-(1-(2,2,2-trifluoroethyl)-1H-indazol-3-yl)benzamide

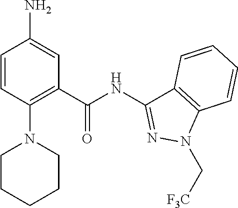 [SMILES: Nc1ccc(N2CCCCC2)c(C(=O)Nc2nn(CC(F)(F)F)c3ccccc23)c1]

To a solution of 5-nitro-2-(piperidin-1-yl)-N-(1-(2,2,2-trifluoroethyl)-1H-indazol-3-yl)benzamide (0.4 g, 894.04 μmol, 1 eq) in EtOAc (8 mL) was added Pd/C (50 mg, 10% purity) under N₂. The mixture was stirred at 25° C. for 1 h under H₂ (15 psi). The mixture was filtered, and the filtrate was concentrated under vacuum to afford the title compound (0.4 g, 757.02 μmol, 84.67% yield, 79% i6 purity) as yellow oil.

LCMS-Y; MS (ESIpos): m/z=418.3 [M+H]⁺.

### Step 4: methyl 2-(2-((4-(piperidin-1-yl)-3-((1-(2,2,2-trifluoroethyl)-1H-indazol-3-yl)carbamoyl)phenyl)carbamoyl)phenyl)acetate

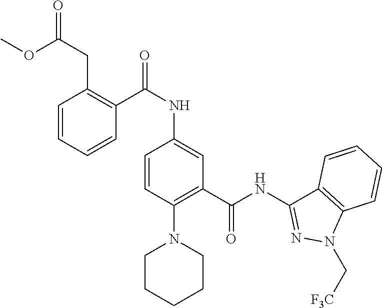 [SMILES: COC(=O)Cc1ccccc1C(=O)Nc1ccc(N2CCCCC2)c(C(=O)Nc2nn(CC(F)(F)F)c3ccccc23)c1]

To a solution of 5-amino-2-(piperidin-1-yl)-N-(1-(2,2,2-trifluoroethyl)-1H-indazol-3-yl)benzamide (0.3 g, 718.69 μmol, 1 eq), DIPEA (185.77 mg, 1.44 mmol, 2 eq) and 2-(2-methoxy-2-oxo-ethyl)benzoic acid (167.47 mg, 862.43 μmol, 1.2 eq) in DCM (10 mL) was added HATU (409.90 mg, 1.08 mmol, 1.5 eq). The mixture was stirred at 25° C. for 16 h. The mixture was concentrated under vacuum. The residue was purified by flash silica gel chromatography (20 g SepaFlash (@Silica Flash Column, Eluent of 5˜20% Ethylacetate/Petroleum ether gradient 40 mL/min) to give the title compound (0.35 g, 454.01 μmol, 63.17% yield, 77% purity) as yellow oil.

LCMS-P; MS (ESIpos): m/z=594.3 [M+H]⁺.

### Step 5: 2-(2-((4-(piperidin-1-yl)-3-((1-(2,2,2-trifluoroethyl)-1H-indazol-3-yl)carbamoyl)phenyl)carbamoyl)phenyl)acetic acid

To a solution of methyl 2-(2-((4-(piperidin-1-yl)-3-((1-(2,2,2-trifluoroethyl)-1H-indazol-3-yl)carbamoyl)phenyl)carbamoyl)phenyl)acetate (150 mg, 194.58 μmol, 77% purity, 1 eq) in toluene (3 mL) was added (Bu₃Sn)₂O (115.99 mg, 194.58 μmol, 1 eq). The mixture was stirred at 90° C. for 16 h. The mixture was concentrated under vacuum. The residue was purified by prep-HPLC (column: Unisil 3-100 C18 Ultra 150*50 mm*3 um; mobile phase: [water(FA)-ACN]; B %: 50%-80%, 5 min) followed by lyophilization to afford the crude product. The crude product was purified by prep-TLC (Ethyl acetate: Petroleum ether=2:1, R₆=0.5) and prep-HPLC (column: Phenomenex Synergi Polar-RP 100*25 mm*4 um; mobile phase: [water(TFA)-ACN]; B %: 46/6-66%, 7 min) followed by lyophilization to the title compound (7.5 mg, 10.27 μmol, 5.28% yield, 95% purity, TFA) as white solid.

¹H NMR (400 MHz, DMSO-d₆) δ=13.50 (br s, 1H), 10.54 (s, 1H), 8.51 (d, J=2.6 Hz, 1H), 8.16 (d, J=8.3 Hz, 1H), 7.95 (dd, J=2.6, 8.7 Hz, 11H), 7.77 (d, J=8.5 Hz, 1H), 7.62 (dd, J=1.2, 7.4 Hz, 1H), 7.55-7.45 (m, 3H), 7.44-7.35 (m, 2H), 7.23-7.16 (m, 1H), 5.39 (q, J=9.1 Hz, 2H), 3.87 (s, 2H), 3.09-2.97 (m, 4H), 1.85-1.77 (m, 4H), 1.62-1.53 (m, 2H).

LCMS-I; HPLC-P; MS (ESIpos): m/z=580.2 [M+H]⁺.

### Example 21: 2-(2-(3-(1-(2,2-Difluoroethyl)-1H-indazole-3-carboxamido)-4-(piperidin-1-yl)benzamido)-5-fluorophenyl)acetic acid F

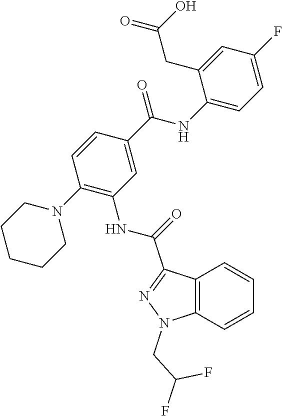 [SMILES: O=C(O)Cc1cc(F)ccc1NC(=O)c1ccc(N2CCCCC2)c(NC(=O)c2nn(CC(F)F)c3ccccc23)c1]

Intermediate 10a (66.4 mg, 115 μmol, 95.3%) was dissolved in DCM (1 mL) and then cooled down to 0° C. HCl in dioxane/water (87.6 mg, 601 μL, 4 molar, 20 Eq, 2.40 mmol) was added portion wise. The mixture was stirred 15 min at 0° C. and then at 25° C. until complete conversion. The solvent was removed under reduce pressure. The remaining crude material was purified by prep-HPLC (Method described herein, Water (Acid): MeCN) to give the title compound (66.4 mg, 115 μmol, 95.3%).

¹H NMR (500 MHz, DMSO-d₆) 5:12.27-12.63 (m, 1H), 10.01-10.04 (m, 1H), 9.97-10.01 (m, 1H), 8.99 (d, 1H), 8.30 (d, 1H), 7.91 (d, 1H), 7.72 (m, 1H), 7.58 (m, 1H), 7.38-7.44 (m, 2H), 7.36 (d, 1H), 7.22 (mn, 1H), 7.16 (mn, 1H), 6.55 (mn, 1H), 5.13 (mn, 2H), 3.67 (s, 2H), 2.85-2.96 (mn, 4H), 1.83 (mn, 4H), 1.57-1.69 (mn, 2H).

LC-MS: Rₜ=1.39 min; MS (ESI+): m/z=580.4 [M+H]⁺.

The following examples were prepared in a methodology analogous to the Example 21 with suitable reagents, precursors and starting materials.

| Compound # | Name | Structure | Analytical Data |
|---|---|---|---|
| 1 | 2-(5-fluoro-2-(N- methyl-4- (piperidin-1-yl)-3- (1-(2,2,2- trifluoroethyl)-1H- indazole-3- carboxamido) benzamido) phenyl)acetic acid | 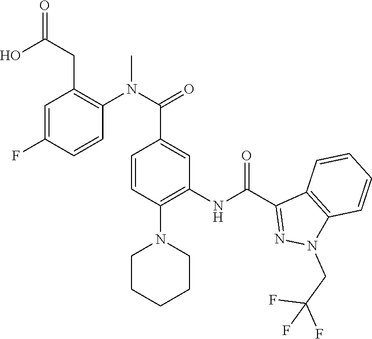 [SMILES: CN(C(=O)c1ccc(N2CCCCC2)c(NC(=O)c2nn(CC(F)(F)F)c3ccccc23)c1)c1ccc(F)cc1CC(=O)O] | ¹H NMR (400 MHz, DMSO- d6) δ: 12.23 − 13.07 (m, 1 H), 9.75 − 10.15 (m, 1 H), 8.39 − 8.69 (m, 1 H), 8.27 − 8.36 (m, 1 H), 7.93 − 8.04 (m, 1 H), 7.58 − 7.65 (m, 1 H), 7.18 − 7.54 (m, 3 H), 6.83 − 7.18 (m, 3 H), 5.57 − 5.72 (m, 2 H), 3.55 − 3.66 (m, 2 H), 3.22 (s, 3 H), 2.68 − 2.95 (m, 4 H), 1.67 − 1.86 (m, 4 H), 1.46 − 1.66 (m, 2 H). UPLC-MS: Rₜ = 1.41 min. MS (ESI+): m/z = 612.8 [M + H]⁺. prep-HPLC (Method described herein, Water (Acid): MeCN, gradient E) |
| 94 | 2-(2-(3-(1- (cyclopropylmethyl)- 1H-indazole-3- carboxamido)-4- (piperidin-1- yl)benzamido)-5- fluorophenyl)acetic acid | 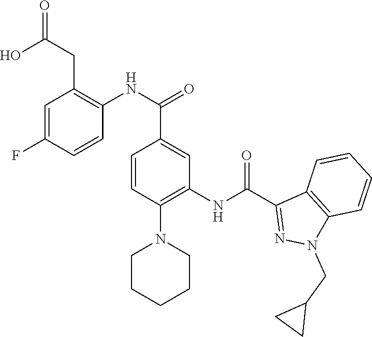 [SMILES: O=C(O)Cc1cc(F)ccc1NC(=O)c1ccc(N2CCCCC2)c(NC(=O)c2nn(CC3CC3)c3ccccc23)c1] | ¹H NMR (DMSO-d6) δ: 11.66-13.29 (m, 1H), 10.42-11.17 (m, 1H), 10.02 (s, 1H), 9.02 (d, 1H), 8.29 (d, 1H), 7.89 (d, 1H), 7.76 (m, 1H), 7.52-7.58 (m, 1H), 7.52 (m, 1H), 7.34-7.39 (m, 1H), 7.33 (d, 1H), 7.16 (m, 1H), 7.12 (m, 1H), 4.46 (d, 2H), 3.56-3.65 (m, 2H), 2.85-2.98 (m, 4H), 1.78- 1.91 (m, 4H), 1.63 (br s, 2H), 1.40 (quinm, 1H), 0.57-0.65 (m, 2H), 0.47- 0.55 (m, 2H). UPLC-MS: Rₜ = 0.92 min. MS (ESI+): m/z = 570.8 [M + H]⁺. |
| 95 | 2-(5-fluoro-2-(3-(1- isobutyl-1H- indazole-3- carboxamido)-4- (piperidin-1- yl)benzamido) phenyl) acetic acid | 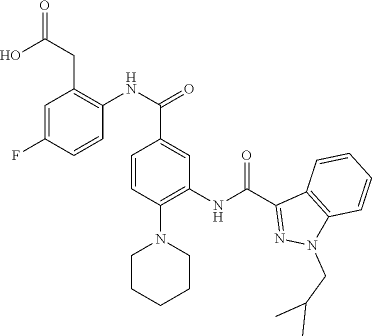 [SMILES: CC(C)Cn1nc(C(=O)Nc2cc(C(=O)Nc3ccc(F)cc3CC(=O)O)ccc2N2CCCCC2)c2ccccc21] | ¹H NMR (400 MHz, DMSO-d6) δ 12.42 (br s, 1H), 10.07 (s, 1H), 9.96 (s, 1H), 8.99 (d, J = 2.0 Hz, 1H), 8.27 (dt, J = 8.2, 1.0 Hz, 1H), 7.88 (d, J = 8.6 Hz, 1H), 7.70 (dd, J = 8.4, 2.0 Hz, 1H), 7.52 (ddd, J = 8.4, 7.0, 1.0 Hz, 1H), 7.42 (dd, J = 8.6, 5.6 Hz, 1H), 7.39 − 7.30 (m, 2H), 7.22 (dd, J = 9.6, 3.0 Hz, 1H), 7.16 (td, J = 8.5, 3.0 Hz, 1H), 4.39 (d, J = 7.1 Hz, 2H), 3.67 (s, 2H), 2.94 − 2.88 (m, 4H), 2.42 − 2.29 (m, 1H), 1.88 − 1.79 (m, 4H), 1.63 (br d, J = 3.5 Hz, 2H), 0.95 (d, J = 6.6 Hz, 6H) LC-MS: Rₜ = 1.57 min. MS (ESI+): m/z = 572.5 [M + H]⁺. |
| 97 | 2-(2-(3-(1- Cyclopropyl-1H- indazole-3- carboxamido)-4- (piperidin-1- yl)benzamido)-5- fluorophenyl)acetic acid | 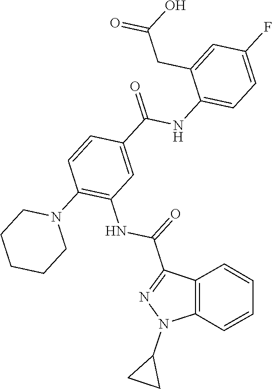 [SMILES: O=C(O)Cc1cc(F)ccc1NC(=O)c1ccc(N2CCCCC2)c(NC(=O)c2nn(C3CC3)c3ccccc23)c1] | ¹H NMR (400 MHz, DMSO- d6) δ: 10.66 − 11.09 (m, 1 H) 10.07 (s, 1 H) 8.93 − 9.11 (m, 1 H) 8.21 − 8.35 (m, 1 H) 7.88 (d, J = 8.36 Hz, 1 H) 7.76 (dd, J = 8.11, 2.03 Hz, 1 H) 7.50 − 7.61 (m, 2 H) 7.29 − 7.46 (m, 2 H) 7.09 − 7.19 (m, 2 H) 3.98 − 4.11 (m, 1 H) 3.59 (s, 2 H) 2.83 − 2.95 (m, 4 H) 1.77 − 1.95 (m, 4 H) 1.65 (br s, 2 H) 1.19 − 1.30 (m, 4 H). UPLC-MS: Rₜ = 0.90 min. MS (ESI+): m/z = 556.7 [M + H]⁺. |
| 98 | 2-(2-(3-(1- (cyclobutylmethyl)- 1H-indazole-3- carboxamido)-4- (piperidin-1- yl)benzamido)-5- fluorophenyl)acetic acid | 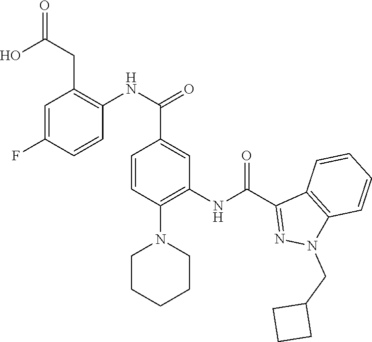 [SMILES: O=C(O)Cc1cc(F)ccc1NC(=O)c1ccc(N2CCCCC2)c(NC(=O)c2nn(CC3CCC3)c3ccccc23)c1] | ¹H NMR (DMSO-d6) δ: 12.43 (br s, 1H), 10.04 (s, 1H), 9.95-10.03 (m, 1H), 9.00 (d, 1H), 8.27 (d, 1H), 7.88 (d, 1H), 7.71 (m, 1H), 7.52 (m, 1H), 7.42 (m, 1H), 7.36 (br d, 1H), 7.34-7.38 (m, 1H), 7.22 (m, 1H), 7.16 (m, 1H), 4.59 (d, 2H), 3.67 (s, 2H), 2.93-3.03 (m, 1H), 2.87-2.95 (m, 2H), 2.05 (br d, 2H), 1.86-1.93 (m, 4H), 1.83-1.88 (m, 4H), 1.65 (br s, 2H) LC-MS: Rₜ = 1.60 min. MS (ESI+): m/z = 584.5 [M + H]⁺. |
| 99 | 2-(5-fluoro-2-(3-(1- (2-hydroxyethyl)- 1H-indazole-3- carboxamido)-4- (piperidin-1- yl)benzamido)phenyl) acetic acid | 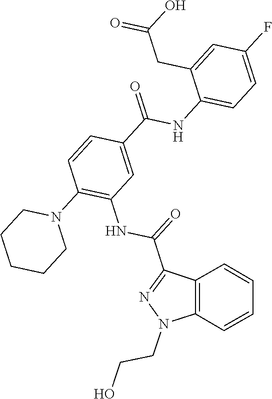 [SMILES: O=C(O)Cc1cc(F)ccc1NC(=O)c1ccc(N2CCCCC2)c(NC(=O)c2nn(CCO)c3ccccc23)c1] | ¹H NMR (600 MHz, DMSO- d6) δ: 12.22-12.63 (m, 1H), 10.04 (s, 1H), 9.97-10.02 (m, 1H), 9.00 (d, 1H), 8.27 (d, 1H), 7.83 (d, 1H), 7.71 (m, 1H), 7.51 (m, 1H), 7.43 (m, 1H), 7.35 (s, 1H), 7.36 (s, 1H), 7.22 (m, 1H), 7.16 (m, 1H), 4.98 (m, 1H), 4.59 (m, 2H), 3.95 (m, 2H), 3.67 (s, 2H), 2.85-2.97 (m, 4H), 1.84 (m, 4H), 1.64 (br s, 2H). UPLC-MS: Rₜ = 1.20 min; MS (ESI+): m/z = 560.7 [M + H]* |
| 104 | 2-(5-fluoro-2-(4- (piperidin-1-yl)-3- (1-((tetrahydro-2H- pyran-4-yl)methyl)- 1H-indazole-3- carboxamido) benzamido) phenyl)acetic acid | 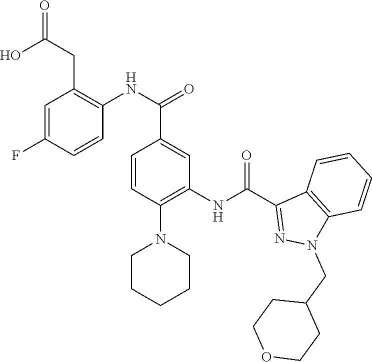 [SMILES: O=C(O)Cc1cc(F)ccc1NC(=O)c1ccc(N2CCCCC2)c(NC(=O)c2nn(CC3CCOCC3)c3ccccc23)c1] | ¹H NMR (DMSO-d6) δ: 12.06-12.72 (m, 1H), 10.04 (s, 2H), 8.99 (d, 1H), 8.25- 8.30 (m, 1H), 7.91 (d, 1H), 7.72 (m, 1H), 7.53 (m, 1H), 7.43 (m, 1H), 7.35-7.39 (m, 1H), 7.36 (d, 1H), 7.22 (m, 1H), 7.16 (m, 1H), 4.48 (d, 2H), 3.80-3.90 (m, 2H), 3.67 (s, 2H), 3.24 (m, 2H), 2.86-2.97 (m, 4H), 2.24- 2.34 (m, 1H), 1.85 (m, 4H), 1.65 (br s, 2H), 1.50 (br m, 2H), 1.35-1.45 (m, 2H). UPLC-MS (method 6): Rₜ = 1.36 min. MS (ESI+): m/z = 614.9 [M + H]⁺. |
| 105 | 2-(5-fluoro-2-(3-(1- (2-methoxyethyl)- 1H-indazole-3- carboxamido)-4- (piperidin-1- yl)benzamido)phenyl) acetic acid | 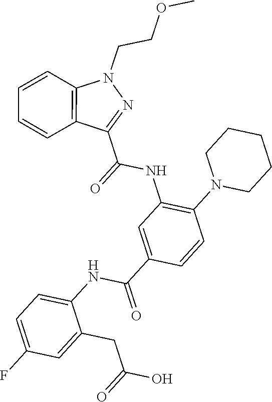 [SMILES: COCCn1nc(C(=O)Nc2cc(C(=O)Nc3ccc(F)cc3CC(=O)O)ccc2N2CCCCC2)c2ccccc21] | ¹H NMR (DMSO-d6) δ: 12.28-12.59 (m, 1H), 10.02 (s, 1H), 9.97-10.00 (m, 1H), 8.99 (d, 1H), 8.27 (d, 1H), 7.86 (d, IH), 7.71 (m, 1H), 7.52 (m, 1H), 7.43 (m, 1H), 7.35-7.39 (m, 1H), 7.36 (d, 1H), 7.22 (m, 1H), 7.16 (m, 1H), 4.73 (m, 2H), 3.91 (m, 2H), 3.67 (s, 2H), 3.24 (s, 3H), 2.86-2.97 (m, 4H), 1.85 (m, 4H), 1.64 (br s, 2H). UPLC-MS: Rₜ = 1.35 min; MS (ESI+): m/z = 574.8 [M + H]⁺. |
| 106 | 2-(5-fluoro-2-(3-(1- (2-hydroxy-2- methylpropyl)-1H- indazole-3- carboxamido)-4- (piperidin-1- yl)benzamido)phenyl) acetic acid | 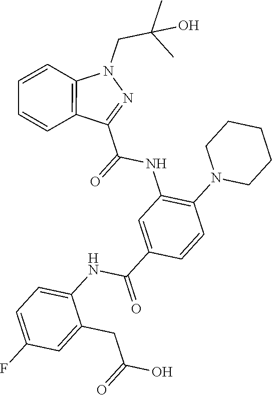 [SMILES: CC(C)(O)Cn1nc(C(=O)Nc2cc(C(=O)Nc3ccc(F)cc3CC(=O)O)ccc2N2CCCCC2)c2ccccc21] | ¹H NMR (600 MHz, DMSO- d6) δ: 12.29-12.55 (m, IH), 10.03-10.06 (m, 1H), 9.98- 10.03 (m, 1H), 9.03 (d, 1H), 8.27 (d, 1H), 7.86 (d, 1H), 7.71 (m, 1H), 7.49 (m, 1H), 7.43 (m, 1H), 7.34-7.38 (m, 2H), 7.31-7.36 (m, 1H), 7.22 (m, 1H), 7.16 (m, 1H), 4.78- 4.86 (m, 1H), 4.38-4.50 (m, 2H), 3.67 (s, 2H), 2.82-2.97 (m, 4H), 1.80-1.87 (m, 4H), 1.58-1.70 (m, 2H), 1.25 (s, 6H). UPLC-MS: Rₜ = 1.30 min; MS (ESI+): m/z = 588.8 [M + H]⁺. Prep-HPLC: Method described herein, acidic eluent, gradient E. |
| 107 | 2-(5-fluoro-2-(4- (piperidin-1-yl)-3- (1-(2,2,2- trifluoroethyl)-1H- pyrazolo[4,3- c]pyridine-3- carboxamido) benzamido) phenyl)acetic acid | 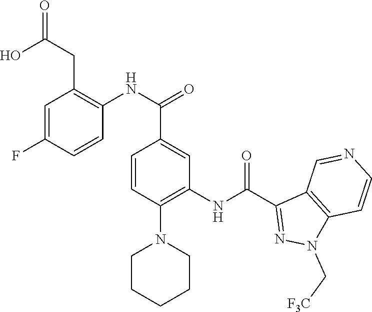 [SMILES: O=C(O)Cc1cc(F)ccc1NC(=O)c1ccc(N2CCCCC2)c(NC(=O)c2nn(CC(F)(F)F)c3ccncc23)c1] | ¹H NMR (400 MHz, DMSO-d6) δ: 12.41 (s, 1H), 10.33 (br s, 1H), 10.03 (s, 1H), 9.55-9.63 (m, 1H), 8.95-9.05 (m, 1H), 8.59- 8.66 (m, 1H), 8.00-8.07 (m, 1H), 7.73-7.81 (m, 1H), 7.43-7.51 (m, 1H), 7.38 (d, J = 8.36 Hz, 1H), 7.11-7.23 (m, 2H), 5.72 (q, J = 9.04 Hz, 2H), 3.64 (s, 2H), 2.85-2.94 (m, 4H), 1.75-1.85 (m, 4H), 1.56-1.68 (m, 2H). UPLC-MS: Rₜ = 0.81 min; MS (ESI+): m/z = 599.8 [M + H]⁺. prep-HPLC (Method described herein, Water (Basic): MeCN, gradient B) |
| 109 | 2-(5-fluoro-2-(3- (imidazo[1,2- a]pyridine-2- carboxamido)-4- (piperidin-1- yl)benzamido) phenyl)acetic acid | 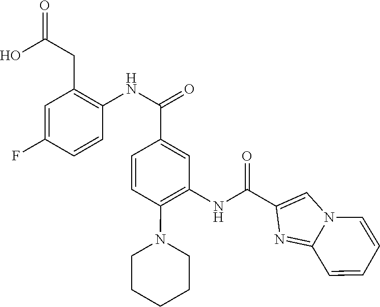 [SMILES: O=C(O)Cc1cc(F)ccc1NC(=O)c1ccc(N2CCCCC2)c(NC(=O)c2cn3ccccc3n2)c1] | ¹H NMR (400 MHz, DMSO-d6) δ: 11.90 (br s, 1H), 10.25 (s, 1H), 8.94- 9.17 (m, 1H), 8.60-8.64 (m, 1H), 8.57-8.59 (m, 1H), 7.79-7.84 (m, 1H), 7.69- 7.75 (m, 1H), 7.64-7.68 (m, 1H), 7.37-7.43 (m, 1H), 7.30 (d, J = 8.36 Hz, 1H), 7.06-7.12 (m, 2H), 7.03 (td, J = 6.78, 1.14 Hz, 1H), 3.49 (s, 2H), 2.86-2.93 (m, 4H), 1.81-1.91 (m, 4H), 1.58- 1.69 (m, 2H). UPLC-MS: Rₜ = 0.79 min; MS (ESI+): m/z = 516.7 [M + H]⁺. prep-HPLC (Method described herein, Water (Basic): MeCN, gradient B) |
| 110 | 2-(5-fluoro-2-(3-(1- methylimidazo[1,5- a]pyridine-3- carboxamido)-4- (piperidin-1- yl)benzamido)phenyl) acetic acid | 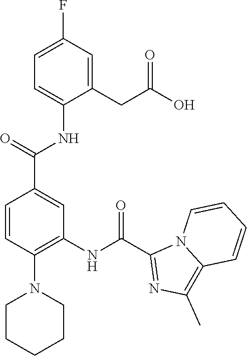 [SMILES: Cc1nc(C(=O)Nc2cc(C(=O)Nc3ccc(F)cc3CC(=O)O)ccc2N2CCCCC2)n2ccccc12] | ¹H NMR (400 MHz, DMSO-d6) δ: 12.41 (br s, 1H), 10.18 (m, 1H), 10.03 (br s, 1H), 9.32-9.42 (m, 1H), 8.87-8.99 (m, 1H), 7.83-7.88 (m, 1H), 7.67- 7.72 (m, 1H), 7.43 (dd, J = 8.62, 5.58 Hz, 1H), 7.31- 7.35 (m, 1H), 7.18-7.24 (m, 1H), 7.08-7.18 (m, 2H), 7.02-7.08 (m, 1H), 3.66 (s, 2H), 2.86-2.96 (m, 4H), 2.55 (s, 3H), 1.80-1.93 (m, 4H), 1.57-1.69 (m, 2H). LC-MS: Rₜ = 1.47 min; MS (ESI+): m/z = 530.4 [M + H]⁺. prep-HPLC (Method described herein, Water (Acidic): MeCN, gradient F) |
| 111 | 2-(5-fluoro-2-(4- (piperidin-1-yl)-3- (1-(2,2,2- trifluorocthyl)-1H- pyrazolo[4,3- b]pyridine-3- carboxamido) benzamido) phenyl)acetic acid | 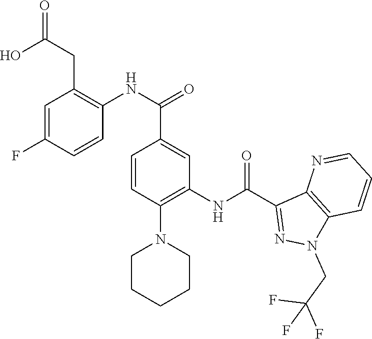 [SMILES: O=C(O)Cc1cc(F)ccc1NC(=O)c1ccc(N2CCCCC2)c(NC(=O)c2nn(CC(F)(F)F)c3cccnc23)c1] | ¹H NMR (400 MHz, DMSO-d6) δ: 12.46 (s, 1 H), 11.27 (s, 1 H), 10.00 (s, 1 H), 9.00 − 9.09 (m, 1 H), 8.82 − 8.90 (m, 1 H), 8.48 − 8.59 (m, 1 H), 7.71 − 7.80 (m, 2 H), 7.36 − 7.48 (m, 2 H), 7.11 − 7.26 (m, 2 H), 5.70 − 5.82 (m, 2 H), 3.67 (s, 2 H), 2.84 − 2.94 (m, 4 H), 1.71 − 1.86 (m, 4 H), 1.53 − 1.67 (m, 2 H). UPLC-MS: Rₜ = 1,29 min; MS (ESI+): m/z = 599.8 [M + H]⁺. prep-HPLC (Method described herein, Water (Acid): MeCN, gradient D) |
| 112 | 2-(5-fluoro-2-(4- (piperidin-1-yl)-3- (1-(2,2,2- trifluoroethyl)-1H- pyrazolo[3,4- c]pyridine-3- carboxamido) benzamido) phenyl)acetic acid | 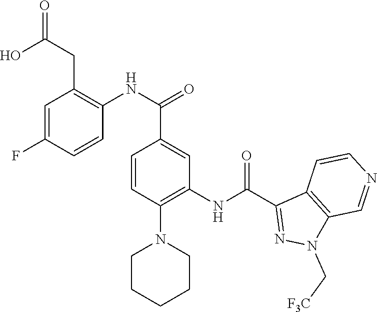 [SMILES: O=C(O)Cc1cc(F)ccc1NC(=O)c1ccc(N2CCCCC2)c(NC(=O)c2nn(CC(F)(F)F)c3cnccc23)c1] | ¹H NMR (400 MHz, DMSO-d6) δ: 12.43 (s, 1 H), 10.16 (br s, 1 H), 10.02 (s, 1 H), 9.45 − 9.55 (m, 1 H), 8.91 − 9.01 (m, 1 H), 8.54 (d, J = 5.32 Hz, 1 H), 8.21 (dd, J = 5.70, 1.39 Hz, 1 H), 7.75 (dd, J = 8.36, 2.03 Hz, 1 H), 7.41 − 7.47 (m, 1 H), 7.38 (d, J = 8.36 Hz, 1 H), 7.21 (dd, J = 9.76, 2.92 Hz, 1 H), 7.15 (td, J = 8.49, 3.04 Hz, 1 H), 5.78 − 5.89 (m, 2 H), 3.66 (s, 2 H), 2.87 − 2.95 (m, 4 H), 1.76 − 1.85 (m, 4 H), 1.54 − 1.69 (m, 2H) UPLC-MS: Rₜ = 0.81 min; MS (ESI+): m/z = 599.7 [M + H]⁺. |
| | | | prep-HPLC (Method |
| | | | described herein, Water |
| | | | (Basic): MeCN, gradient |
| | | | B) |
| 113 | 2-(5-fluoro-2-(4- (piperidin-1-yl)-3- (1-(2,2,2- trifluoroethyl)-1H- pyrazolo[3,4- b]pyridine-3- carboxamido) benzamido) phenyl)acetic acid | 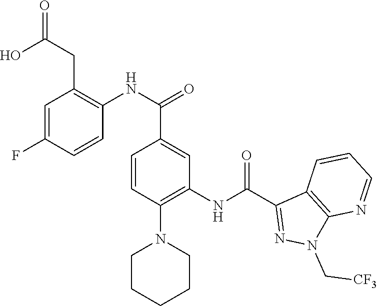 [SMILES: O=C(O)Cc1cc(F)ccc1NC(=O)c1ccc(N2CCCCC2)c(NC(=O)c2nn(CC(F)(F)F)c3ncccc23)c1] | ¹H NMR (400 MHz, DMSO-d6) δ: 12.45 (s, 1 H), 10.31 (br s, 1 H), 10.04 (s, 1 H), 8.96 − 8.99 (m, 1 H), 8.77 − 8.81 (m, 1 H), 8.69 − 8.74 (m, 1 H), 7.73 − 7.79 (m, 1 H), 7.53 − 7.59 (m, 1 H), 7.47 (dd, J = 8.62, 5.58 Hz, 1 H), 7.38 (d, J = 8.36 Hz, 1 H), 7.11 − 7.23 (m, 2 H), 5.58 (q, J = 9.04 Hz, 2 H), 3.64 (s, 2 H), 2.86 − 2.95 (m, 4 H), 1.75 − 1.88 (m, 4 H), 1.54 − 1.70 (m, 2 H). UPLC-MS: Rₜ = 0.83 min; MS (ESI+): m/z = 599.7 [M + H]⁺. prep-HPLC (Method described herein, Water |
| | | | (Basic): MeCN, gradient C) |
| 115 | 2-(5-fluoro-2-(3-(4- phenyl-1-(2,2,2- trifluoroethyl)-1H- pyrazole-3- carboxamido)-4- (piperidin-1- yl)benzamido) phenyl)acetic acid | 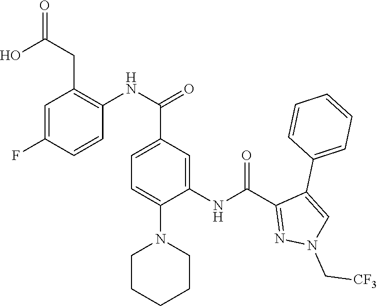 [SMILES: O=C(O)Cc1cc(F)ccc1NC(=O)c1ccc(N2CCCCC2)c(NC(=O)c2nn(CC(F)(F)F)cc2-c2ccccc2)c1] | ¹H NMR (400 MHz, DMSO-d6) δ: 11.49 (s, 1 H), 9.92 (s, 1 H), 8.86 − 8.92 (m, 1 H), 8.27 (s, 1 H), 7.75 − 7.83 (m, 1 H), 7.59 − 7.68 (m, 3 H), 7.38 − 7.45 (m, 2 H), 7.28 − 7.37 (m, 2 H), 7.03 − 7.13 (m, 2 H), 5.32 (q, J = 9.04 Hz, 2 H), 3.50 (s, 2 H), 2.81 − 2.92 (m, 4 H), 1.72 − 1.84 (m, 4 H), 1.54 − 1.65 (m, 2 H). LC-MS: Rₜ = 1.45 min; MS (ESI+): m/z = 624.6 prep-HPLC (Method described herein, Water (Basic): MeCN, gradient C) |
| 116 | 2-(5-fluoro-2-(3-(5- phenyl-1-(2,2,2- trifluoroethyl)-1H- pyrazole-3- carboxamido)-4- (piperidin-1- yl)benzamido) phenyl)acetic acid | 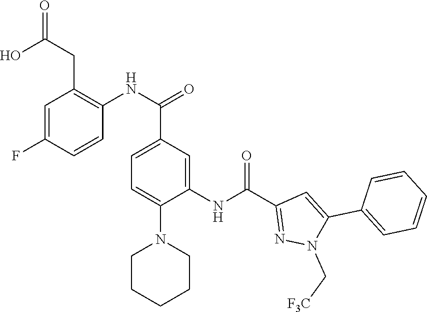 [SMILES: O=C(O)Cc1cc(F)ccc1NC(=O)c1ccc(N2CCCCC2)c(NC(=O)c2cc(-c3ccccc3)n(CC(F)(F)F)n2)c1] | ¹H NMR (400 MHz, DMSO-d6) δ: 12.42 (s, 1 H), 9.97 (s, 1 H), 9.91 (s, 1 H), 8.86 − 8.99 (m, 1 H), 7.66 − 7.77 (m, 1 H), 7.52 − 7.60 (m, 5 H), 7.41 (dd, J = 8.62, 5.58 Hz, 1 H), 7.36 (d, J = 8.11 Hz, 1 H), 7.19 − 7.24 (m, 1 H), 7.15 (td, J = 8.49, 3.04 Hz, 1 H), 7.04 (s, 1 H), 5.09 − 5.24 (m, 2 H), 3.66 (s, 2 H), 2.84 − 2.95 (m, 4 H), 1.74 − 1.86 (m, 4 H), 1.54 − 1.69 (m, 2 H). ¹⁹F NMR (377 MHz, DMSO-d6) δ: −68.97 (t, J = 8.58 Hz, 3F), −118.77 − −115.93 (m, 1F). LC-MS: R: = 1.49 min; MS |
| | | | (ESI+): m/z = 624.3 |
| | | | [M + H]⁺. |
| | | | prep-HPLC (Method |
| | | | described herein, Water |
| | | | (Acidic): MeCN, gradient F) |
| 118 | 2-(5-fluoro-2-(4- (piperidin-1-yl)-3- (1- ((tetrahydrofuran-3- yl)methyl)-1H- indazole-3- carboxamido) benzamido) phenyl)acetic acid | 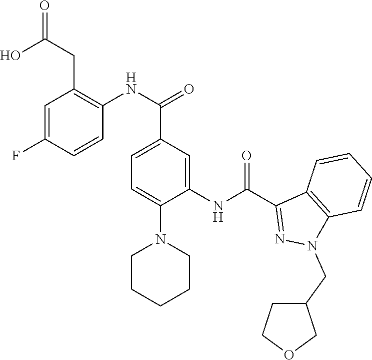 [SMILES: O=C(O)Cc1cc(F)ccc1NC(=O)c1ccc(N2CCCCC2)c(NC(=O)c2nn(CC3CCOC3)c3ccccc23)c1] | ¹H NMR (400 MHz, DMSO-d6) δ: 10.46 (br s, 1 H), 10.02 (s, 1 H), 9.01 (d, J = 2.03 Hz, 1 H), 8.26-8.31 (m, 1 H), 7.89-7.93 (m, 1 H), 7.74 (dd, J = 8.24, 2.15 Hz, 1 H), 7.47 − 7.57 (m, 2 H), 7.33 − 7.41 (m, 2 H), 7.11 − 7.22 (m, 2 H), 4.57 (d, J = 7.35 Hz, 2 H), 3.80 − 3.87 (m, 1 H), 3.74 − 3.80 (m, 1 H), 3.59 − 3.71 (m, 4 H), 2.87 − 2.98 (m, 5 H), 1.94 − 2.04 (m, 1 H), 1.79 − 1.88 (m, 4 H), 1.71 − 1.80 (m, 1 H), 1.57 − 1.67 (m, 2 H). LC-MS: Rₜ = 1.39 min; MS (ESI+): m/z = 600.4 [M + H]⁺. prop-HPLC (Method described herein, Water (Basic): MeCN, gradient C) |
| 119 | 2-(5-fluoro-2-(4- (piperidin-1-yl)-3- (1- ((tetrahydrofuran-2- yl)methyl)-1H- indazole-3- carboxamido) benzamido) phenyl)acetic acid | 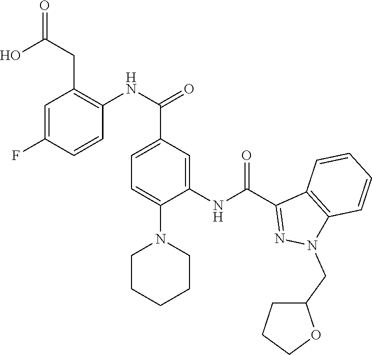 [SMILES: O=C(O)Cc1cc(F)ccc1NC(=O)c1ccc(N2CCCCC2)c(NC(=O)c2nn(CC3CCCO3)c3ccccc23)c1] | ¹H NMR δ: MHz, DMSO-d6) δ: 10.57 (s, 1 H), 10.04 (s, 1 H), 8.97 − 9.05 (m, 1 H), 8.26 (d, J = 8.11 Hz, 1 H), 7.86 (d, J = 8.62 Hz, 1 H), 7.74 (dd, J = 8.11, 2.03 Hz, 1 H), 7.47 − 7.56 (m, 2 H), 7.33 − 7.43 (m, 2 H), 7.09 − 7.23 (m, 2 H), 4.61 (d, J = 5.58 Hz, 2 H), 4.36 − 4.45 (m, 1 H), 3.68 − 3.75 (m, 1 H), 3.58 − 3.66 (m, 3 H), 2.87 − 2.96 (m, 4 H), 1.96 − 2.08 (m, 1 H), 1.74 − 1.89 (m, 7 H), 1.62 (br s, 2 H). LC-MS: Rₜ = 1.44 min; MS (ESI+): m/z = 600.5 [M + H]⁺. prep-HPLC (Method described herein, Water (Basic): MeCN, gradient C) |
| 121 | 2-(5-fluoro-2-(4- (piperidin-1-yl)-3- (1-(3,3,3- trifluoropropyl)- 1H-indazole-3- carboxamido) benzamido) phenyl)acetic acid | 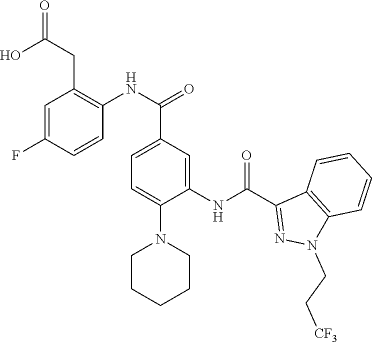 [SMILES: O=C(O)Cc1cc(F)ccc1NC(=O)c1ccc(N2CCCCC2)c(NC(=O)c2nn(CCC(F)(F)F)c3ccccc23)c1] | ¹H NMR (400 MHz, DMSO-d6) δ: 12.41 (s, 1H), 10.08 (s, 1H), 10.03 (br s, 1H), 8.97-9.01 (m, 1H), 8.26-8.33 (m, 1H), 7.88- 7.96 (m, 1H), 7.69-7.75 (m, 1H), 7.53-7.60 (m, 1H), 7.35-7.47 (m, 3H), 7.12- 7.25 (m, 2H), 4.86 (t, J = 6.59 Hz, 2H), 3.66 (s, 2H), 3.03-3.17 (m, 2H), 2.86-2.96 (m, 4H), 1.78- 1.90 (m, 4H), 1.58-1.69 (m, 2H). ¹⁹F NMR (377 MHz, DMSO-d6) δ: − 65.60 − −63.19 (m, 3F), −119.06 − −116.21 (m, 1F) UPLC-MS: Rₜ = 0.93 min; MS (ESI+): m/z = 612.7 [M + H]⁺. prep-HPLC (Method described herein, Water (Acid): MeCN, gradient E) |
| 123 | 2-(2-(3-(1-(but-2- yn-1-yl)-1H- indazole-3- carboxamido)-4- (piperidin-1- yl)benzamido)-5- fluorophenyl)acetic acid | 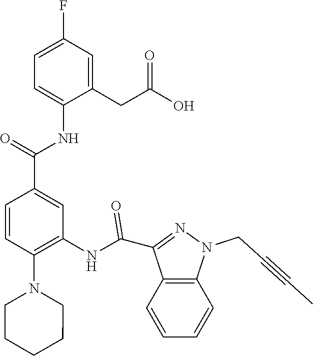 [SMILES: CC#CCn1nc(C(=O)Nc2cc(C(=O)Nc3ccc(F)cc3CC(=O)O)ccc2N2CCCCC2)c2ccccc21] | ¹H NMR (400 MHz, DMSO-d6) δ: 12.42 (br s, 1 H), 9.95 − 10.06 (m, 2 H), 8.91 − 9.01 (m, 1 H), 8.29 (d, J = 8.11 Hz, 1 H), 7.90 (d, J = 8.62 Hz, 1 H), 7.72 (dd, J = 8.36, 2.03 Hz, 1 H), 7.53 − 7.59 (m, 1 H), 7.34 − 7.45 (m, 3 H), 7.12 − 7.24 (m, 2 H), 5.44 − 5.47 (m, 2 H), 3.67 (s, 2 H), 2.86 − 2.97 (m, 4 H), 1.80 − 1.91 (m, 7 H), 1.58 − 1.69 (m, 2 H). LC-MS: Rₜ = 1.47 min; MS (ESI+): m/z = 568.4 [M + H]⁺. prep-HPLC (Method described herein, Water (Acid): MeCN, gradient F) |
| 124 | 2-(5-fluoro-2-(3-(1- ((5-methylisoxazol- 3-yl)methyl)-1H- indazole-3- carboxamido)-4- (piperidin-1- yl)benzamido) phenyl)acetic acid | 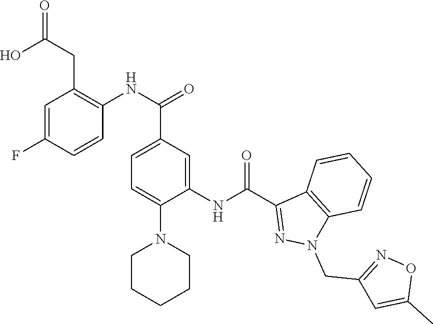 [SMILES: Cc1cc(Cn2nc(C(=O)Nc3cc(C(=O)Nc4ccc(F)cc4CC(=O)O)ccc3N3CCCCC3)c3ccccc32)no1] | ¹H NMR (400 MHz, DMSO-d6) δ: 12.42 (br s, 1 H), 10.04 (br s, 1 H), 9.97 (s, 1 H), 8.92 − 9.01 (m, 1 H), 8.27-8.32 (m, 1 H), 7.90 − 7.98 (m, 1 H), 7.71 (dd, J = 8.24, 2.15 Hz, 1 H), 7.55- 7.60 (m, 1 H), 7.37 − 7.46 (m, 2 H), 7.34 (d, J = 8.36 Hz, 1 H), 7.21 (dd, J = 9.63, 3.04 Hz, 1 H), 7.15 (td, J = 8.62, 3.04 Hz, 1 H), 6.09- 6.12 (m, 1 H), 5.87 (s, 2 H), 3.66 (s, 2 H), 2.81 − 2.91 (m, 4 H), 2.34-2.36 (m, 3 H), 1.66 − 1.77 (m, 4 H), 1.59 (br d, J = 3.55 Hz, 2 H). LC-MS: Rₜ = 1.40 min; MS (ESI+): m/z = 611.4 prep-HPLC (Method described herein, Water |
| | | | (Acid): MeCN, gradient D) |
| 125 | 2-(5-fluoro-2-(3-(1- ((5-methyl-1,2,4- oxadiazol-3- yl)methyl)-1H- indazole-3- carboxamido)-4- (piperidin-1- yl)benzamido) phenyl)acetic acid | 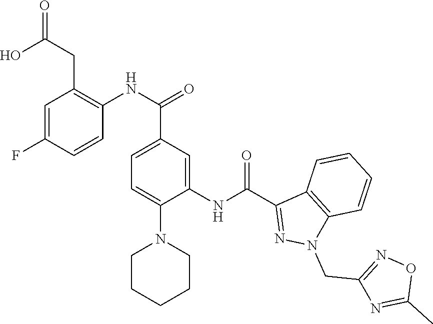 [SMILES: Cc1nc(Cn2nc(C(=O)Nc3cc(C(=O)Nc4ccc(F)cc4CC(=O)O)ccc3N3CCCCC3)c3ccccc32)no1] | ¹H NMR (400 MHz, DMSO-d6) δ: 12.42 (br s, 1 H), 10.02 (br s, 1 H), 9.95 (s, 1 H), 8.90 − 8.99 (m, 1 H), 8.24 − 8.36 (m, 1 H), 7.97 (d, J = 8.62 Hz, 1 H), 7.68 − 7.73 (m, 1 H), 7.57- 7.62 (m, 1 H), 7.39 − 7.45 (m, 2 H), 7.33 (d, J = 8.36 Hz, 1 H), 7.21 (dd, J = 9.76, 2.92 Hz, 1 H), 7.15 (td, J = 8.49, 3.04 Hz, 1 H), 6.00 (s, 2 H), 3.66 (s, 2 H), 2.80 − 2.89 (m, 4 H), 2.55 (s, 3 H), 1.63-1.71 (m, 4H), 1.53 − 1.62 (m, 2 H). LC-MS: Rₜ = 1.30 min; MS (ESI+): m/z = 612.1 [M + H]⁺. prep-HPLC (Method described herein, Water |
| | | | (Acid): MeCN, gradient D) |
| 132 | 2-(2-(3-(1-((1,4- dioxan-2- yl)methyl)-1H- indazole-3- carboxamido)-4- (piperidin-1- yl)benzamido)-5- fluorophenyl)acetic acid | 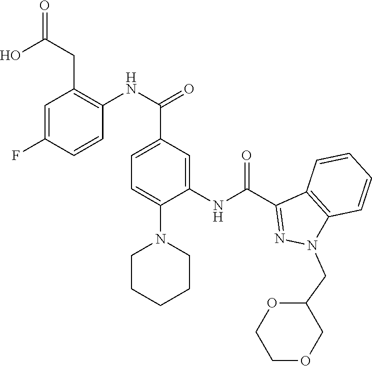 [SMILES: O=C(O)Cc1cc(F)ccc1NC(=O)c1ccc(N2CCCCC2)c(NC(=O)c2nn(CC3COCCO3)c3ccccc23)c1] | ¹H NMR (400 MHz, DMSO-d6) δ: 10.47 (br s, 1 H), 10.01 (s, 1 H), 8.97- 9.01 (m, 1 H), 8.27 (d, J = 8.11 Hz, 1 H), 7.85 (d, J = 8.62 Hz, 1 H), 7.72-7.77 (m, 1 H), 7.46 − 7.57 (m, 2 H), 7.33 − 7.41 (m, 2 H), 7.10 − 7.23 (m, 2 H), 4.56 − 4.69 (m, 2 H), 4.08 − 4.16 (m, 1 H), 3.84 − 3.92 (m, 1 H), 3.60 − 3.75 (m, 4 H), 3.40 − 3.56 (m, 3 H), 2.84 − 2.98 (m, 4 H), 1.78 − 1.90 (m, 4 H), 1.57 − 1.70 (m, 2 H). LC-MS: R: = 1.37 min; MS (ESI+): m/z = 616.4 [M + H]⁺. prep-HPLC (Method described herein, Water (Basic): MeCN, gradient B) |
| 133 | 2-(5-fluoro-2-(4- (piperidin-1-yl)-3- (1-((tetrahydro-2H- pyran-3-yl)methyl)- 1H-indazole-3- carboxamido) benzamido) phenyl)acetic acid | 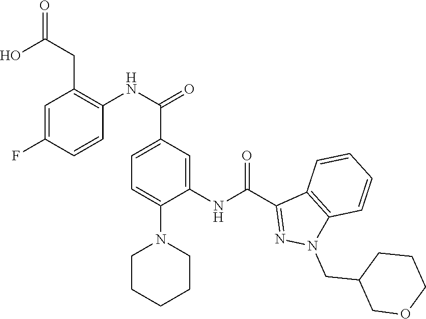 [SMILES: O=C(O)Cc1cc(F)ccc1NC(=O)c1ccc(N2CCCCC2)c(NC(=O)c2nn(CC3CCCOC3)c3ccccc23)c1] | ¹H NMR (400 MHz, DMSO-d6) δ: 12.42 (br s, 1 H), 10.05 (s, 1 H), 10.00 (s, 1 H), 8.96 − 9.02 (m, 1 H), 8.24 − 8.30 (m, 1 H), 7.86 − 7.91 (m, 1 H), 7.68 − 7.73 (m, 1 H), 7.51-7.56 (m, 1 H), 7.34 − 7.45 (m, 3 H), 7.20 − 7.24 (m, 1 H), 7.12 − 7.19 (m, 1 H), 4.40 − 4.55 (m, 2 H), 3.69 − 3.78 (m, 2 H), 3.66 (s, 2 H), 3.26 − 3.41 (m, 2 H), 2.85 − 2.97 (m, 4 H), 2.24 − 2.37 (m, 1 H), 1.79 − 1.90 (m, 4 H), 1.71 − 1.79 (m, 1 H), 1.58 − 1.70 (m, 3 H), 1.27 − 1.52 (m, 2 H). LC-MS: R: = 1.46 min; MS (ESI+): m/z = 614.6[M + H]⁺. prep-HPLC (Method |
| | | | described herein, Water |
| | | | (Acid): MeCN, gradient E) |
| 134 | 2-(5-fluoro-2-(3-(3- phenyl-1-(2,2,2- trifluoroethyl)-1H- pyrazole-5- carboxamido)-4- (piperidin-1- yl)benzamido)phenyl) acetic acid | 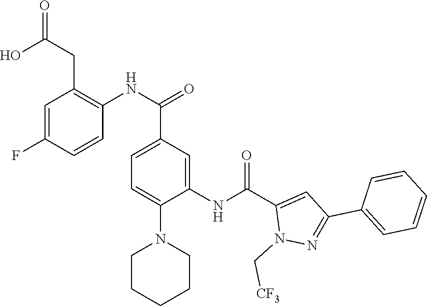 [SMILES: O=C(O)Cc1cc(F)ccc1NC(=O)c1ccc(N2CCCCC2)c(NC(=O)c2cc(-c3ccccc3)nn2CC(F)(F)F)c1] | ¹H NMR (400 MHz, DMSO-d6) δ: 12.41 (br s, 1 H), 10.00 (s, 1 H), 9.77 (s, 1 H), 8.22 (d, J = 2.03 Hz, 1 H), 7.81 − 7.87 (m, 3 H), 7.71 (s, 1 H), 7.48 − 7.54 (m, 2 H), 7.37 − 7.45 (m, 2 H), 7.26 (d, J = 8.62 Hz, 1 H), 7.21 (dd, J = 9.63, 3.04 Hz, 1 H), 7.14 (td, J = 8.49, 3.04 Hz, 1 H), 5.62 (q, J = 9.04 Hz, 2 H), 3.65 (s, 2 H), 2.85 − 2.98 (m, 4 H), 1.60 − 1.72 (m, 4 H), 1.49 − 1.57 (m, 2 H) UPLC-MS: Rₜ = 0.92 min; MS (ESI+): m/z = 624.7 [M + H]⁺. prep-HPLC (Method described herein, Water (Acid): MeCN, gradient F) |
| 136 | (S)-2-(5-fluoro-2- (4-(piperidin-1-yl)- 3-(1- ((tetrahydrofuran-2- yl)methyl)-1H- indazole-3- carboxamido) benzamido) phenyl)acetic acid | 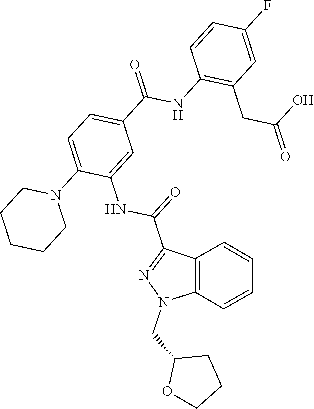 [SMILES: O=C(O)Cc1cc(F)ccc1NC(=O)c1ccc(N2CCCCC2)c(NC(=O)c2nn(C[C@@H]3CCCO3)c3ccccc23)c1] | ¹H NMR (400 MHz, DMSO-d6) δ: 12.41 (br s, 1 H), 10.05 (s, 1 H), 9.97 (s, 1 H), 8.94 − 9.02 (m, 1 H), 8.21 − 8.33 (m, 1 H), 7.83 − 7.91 (m, 1 H), 7.67 − 7.75 (m, 1 H), 7.49 − 7.55 (m, 1 H), 7.39 − 7.45 (m, 1 H), 7.33 − 7.39 (m, 2 H), 7.22 (dd, J = 9.63, 3.04 Hz, 1 H), 7.13 − 7.19 (m, 1 H), 4.61 (d, J = 5.58 Hz, 2 H), 4.36 − 4.46 (m, 1 H), 3.58 − 3.76 (m, 4 H), 2.86 − 2.96 (m, 4 H), 1.98 − 2.06 (m, 1 H), 1.74 − 1.89 (m, 7 H), 1.57 − 1.68 (m, 2 H). UPLC-MS: Rₜ = 1.39 min; MS (ESI+): m/z = 600.6 [M + H]⁺. prep-HPLC (Method described herein, Water (Acid): MeCN, gradient D) |
| 137 | (R)-2-(5-fluoro-2- (4-(piperidin-1-yl)- 3-(1- ((tetrahydrofuran-2- yl)methyl)-1H- indazole-3- carboxamido) benzamido) phenyl)acetic acid | 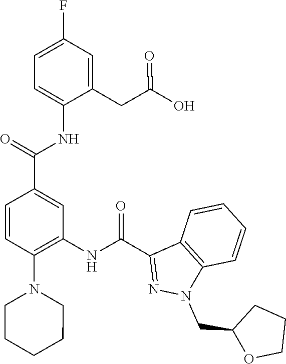 [SMILES: O=C(O)Cc1cc(F)ccc1NC(=O)c1ccc(N2CCCCC2)c(NC(=O)c2nn(C[C@H]3CCCO3)c3ccccc23)c1] | ¹H NMR (400 MHz, DMSO-d6) δ: 10.72 (br s, 1 H), 10.03 (s, 1 H), 8.98 − 9.03 (m, 1 H), 8.23 − 8.31 (m, 1 H), 7.84 − 7.89 (m, 1 H), 7.72 − 7.78 (m, 1 H), 7.48 − 7.56 (m, 2 H), 7.32 − 7.39 (m, 2 H), 7.10 − 7.20 (m, 2 H), 4.59 − 4.64 (m, 2 H), 4.35 − 4.45 (m, 1 H), 3.57 − 3.74 (m, 4 H), 2.85 2.96 (m, 4 H), 1.97 − 2.07 (m, 1 H), 1.75 − 1.88 (m, 7 H), 1.59 − 1.67 (m, 2 H). LC-MS: Rₜ = 1.4 min; MS (ESI+): m/z = 600.1 [M + H]⁺. prep-HPLC (Method described herein, Water (Basic): MeCN, gradient C) |
| 138 | 2-(5-fluoro-2-(3-(5- fluoro-1-(2,2,2- trifluoroethyl)-1H- indazole-3- carboxamido)-4- (piperidin-1- yl)benzamido) phenyl)acetic acid | 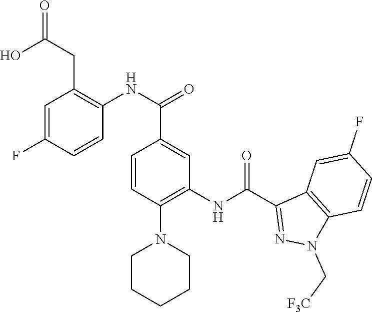 [SMILES: O=C(O)Cc1cc(F)ccc1NC(=O)c1ccc(N2CCCCC2)c(NC(=O)c2nn(CC(F)(F)F)c3ccc(F)cc23)c1] | ¹H NMR (400 MHz, DMSO-d6) δ: 12.41 (s, 1 H), 9.95 − 10.04 (m, 2 H), 8.96 (d, J = 2.03 Hz, 1 H), 8.05 − 8.11 (m, 1 H), 7.96 (dd, J = 8.62, 2.28 Hz, 1 H), 7.73 (dd, J = 8.36, 2.03 Hz, 1 H), 7.54 − 7.61 (m, 1 H), 7.40 − 7.45 (m, 1 H), 7.37 (d, J = 8.36 Hz, 1 H), 7.19 − 7.24 (m, 1 H), 7.13 − 7.19 (m, 1 H), 5.63 − 5.75 (m, 2 H), 3.67 (s, 2 H), 2.85 − 2.95 (m, 4 H), 1.75 − 1.86 (m, 4 H), 1.55 − 1.67 (m, 2 H). ¹⁹F NMR (377 MHz, DMSO-d6) δ: −69.79 (t, J = 9.16 Hz, 3 F), −118.23 − −116.81 (m, 1 F), −118.84 − −119.03 (m, 1 F) |
| | | | LC-MS: R: = 1.40 min; MS |
| | | | (ESI+): m/z = 616.0 [M + H]⁺. |
| | | | prep-HPLC (Method |
| | | | described herein, Water |
| | | | (Acid): MeCN, gradient E) |
| 140 | 2-(5-fluoro-2-(3-(5- methoxy-1-(2,2,2- trifluoroethyl)-1H- indazole-3- carboxamido)-4- (piperidin-1- yl)benzamido) phenyl)acetic acid | 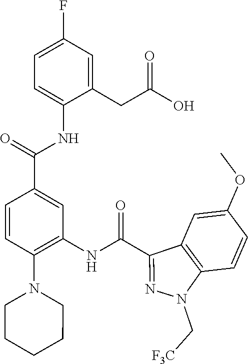 [SMILES: COc1ccc2c(c1)c(C(=O)Nc1cc(C(=O)Nc3ccc(F)cc3CC(=O)O)ccc1N1CCCCC1)nn2CC(F)(F)F] | ¹H NMR (400 MHz, DMSO-d6) δ: 12.41 (br s, 1 H), 10.01 (br s, 1 H), 9.96 (s, 1 H), 8.94 − 9.05 (m, 1 H), 7.91 (d, J = 9.13 Hz, 1 H), 7.70 − 7.75 (m, 1 H), 7.66 − 7.69 (m, 1 H), 7.39 − 7.44 (m, 1 H), 7.34 − 7.38 (m, 1 H), 7.24 − 7.29 (m, 1 H), 7.19 − 7.24 (m, 1 H), 7.12 − 7.19 (m, 1 H), 5.62 (q, J = 9.04 Hz, 2 H), 3.86 (s, 3 H), 3.66 (s, 2 H), 2.85 − 2.95 (m, 4 H), 1.76 − 1.86 (m, 4 H), 1.58 − 1.69 (m, 2 H). LC-MS: Rₜ = 1.40 min; MS (ESI+): m/z = 628.1 [M + H]⁺. prep-HPLC (Method described herein, Water (Acid): MeCN, gradient E) |
| 141 | 2-(5-fluoro-2-(3-(4- methyl-1-(2,2,2- trifluoroethyl)-1H- indazole-3- carboxamido)-4- (piperidin-1- yl)benzamido) phenyl)acetic acid | 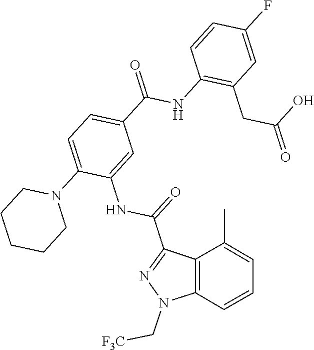 [SMILES: Cc1cccc2c1c(C(=O)Nc1cc(C(=O)Nc3ccc(F)cc3CC(=O)O)ccc1N1CCCCC1)nn2CC(F)(F)F] | ¹H NMR (400 MHz, DMSO-d6) δ: 12.41 (br s, 1 H), 10.08 (s, 1 H), 9.98 (s, 1 H), 8.91 − 9.01 (m, 1 H), 7.77 − 7.83 (m, 1 H), 7.72 (dd, J = 8.36, 2.03 Hz, 1 H), 7.46 − 7.51 (m, 1 H), 7.39 − 7.45 (m, 1 H), 7.34 − 7.39 (m, 1 H), 7.12 − 7.25 (m, 3 H), 5.57 − 5.69 (m, 2 H), 3.67 (s, 2 H), 2.85 − 2.94 (m, 7 H), 1.73 − 1.86 (m, 4 H), 1.54 − 1.65 (m, 2 H). UPLC-MS: Rₜ = 1.45 min; MS (ESI+): m/z = 612.6 [M + H]⁺. prep-HPLC (Method described herein, Water (Acid): MeCN, gradient F) |
| 143 | 2-(5-fluoro-2-(3-(6- methyl-1-(2,2,2- trifluoroethyl)-1H- indazole-3- carboxamido)-4- (piperidin-1- yl)benzamido) phenyl)acetic acid | 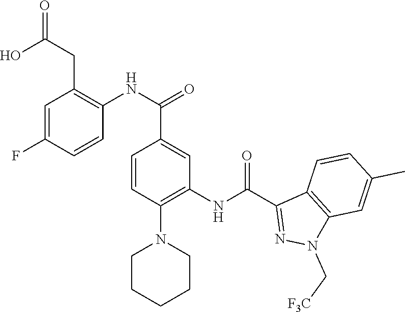 [SMILES: Cc1ccc2c(C(=O)Nc3cc(C(=O)Nc4ccc(F)cc4CC(=O)O)ccc3N3CCCCC3)nn(CC(F)(F)F)c2c1] | ¹H NMR (400 MHz, DMSO-d6) δ: 12.42 (br s, 1 H), 9.91 − 10.08 (m, 2 H), 8.91 − 9.03 (m, 1 H), 8.18 (d, J = 8.36 Hz, 1 H), 7.77 − 7.81 (m, 1 H), 7.69 − 7.75 (m, 1 H), 7.39 − 7.45 (m, 1 H), 7.34 − 7.39 (m, 1 H), 7.25 − 7.29 (m, 1 H), 7.19 − 7.25 (m, 1 H), 7.12 − 7.19 (m, 1 H), 5.53 − 5.66 (m, 2 H), 3.67 (s, 2 H), 2.83 − 2.95 (m, 4 H), 2.50 (s, 3H, behind DMSO), 1.74 − 1.88 (m, 4 H), 1.56 − 1.65 (m, 2 H). LC-MS: Rₜ = 1.4 min; MS (ESI+): m/z = 612.0 [M + H]⁺. prep-HPLC (Method described herein, Water (Acid): MeCN, gradient F) |
| 145 | 2-(5-fluoro-2-(4- (piperidin-1-yl)-3- (1-(2,2,2- trifluoroethyl)- 1,4,5,6- tetrahydrocyclopenta [c]pyrazole-3- carboxamido) benzamido) phenyl)acetic acid | 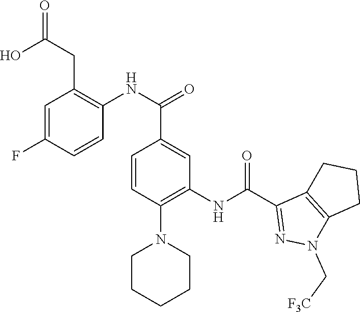 [SMILES: O=C(O)Cc1cc(F)ccc1NC(=O)c1ccc(N2CCCCC2)c(NC(=O)c2nn(CC(F)(F)F)c3c2CCC3)c1] | ¹H NMR (400 MHz. DMSO-d6) δ: 12.36 (br s, 1 H), 9.97 (s, 1 H), 9.67 (s, 1 H), 8.85-8.88 (m, 1 H), 7.63 − 7.73 (m, 1 H), 7.39 (dd, J = 8.74, 5.70 Hz, 1 H), 7.31 (d, J = 8.36 Hz, 1 H), 7.20 (dd, J = 9.76, 2.91 Hz, 1 H), 7.14 (td, J = 8.55, 2.92 Hz, 1 H), 5.17 (q, J = 9.04 Hz, 2 H), 3.64 (s, 2 H), 2.73 − 2.89 (m, 8 H), 2.52 − 2.59 (m, 2 H), 1.71 − 1.83 (m, 4 H), 1.52 − 1.63 (m, 2 H). ¹⁹F NMR (377 MHz, DMSO-d6) δ: − 70.12 (t, J = 9.16 Hz, 3 F), −119.47 − −116.14 (m, 1 F). LC-MS: Rₜ = 1.3 min; MS (ESI+): m/z = 588.1 [M + H]⁺. |
| | | | prep-HPLC (Method |
| | | | described herein, Water |
| | | | (Acid): MeCN, gradient E) |
| 146 | 2-(5-fluoro-2-(4- (piperidin-1-yl)-3- (1-(1,1,1- trifluoropropan-2- yl)-1H-indazole-3- carboxamido) benzamido) phenyl)acetic acid | 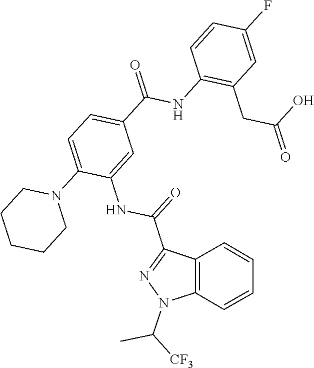 [SMILES: CC(n1nc(C(=O)Nc2cc(C(=O)Nc3ccc(F)cc3CC(=O)O)ccc2N2CCCCC2)c2ccccc21)C(F)(F)F] | ¹H NMR (600 MHz, DMSO-d6) δ: 12.11-12.67 (m, 1H), 10.14 (s, 1H), 10.01 (s, 1H), 9.03 (d, 1H), 8.34 (d, 1H), 8.02 (d, 1H), 7.73 (dd, 1H), 7.61 (ddd, 1H), 7.42-7.47 (m, 1H), 7.41-7.44 (m, 1H), 7.39 (d, 1H), 7.22 (dd, 1H), 7.16 (td, 1H), 6.16 (spt, 1H), 3.62- 3.72 (m, 2H), 2.81-2.98 (m, 4H), 1.93 (d, 3H), 1.76-1.88 (m, 4H), 1.57-1.69 (m, 2H). UPLC-MS: Rₜ = 1.45 min; MS (ESI+): m/z = 612.6 [M + H]⁺. prep-HPLC (Method described herein, Water (Acid): MeCN, gradient F) |
| 148 | 2-(5-fluoro-2-(4- (piperidin-1-yl)-3- (1-(1-(tetrahydro- 2H-pyran-4- yl)ethyl)-1H- indazole-3- carboxamido) benzamido) phenyl)acetic acid | 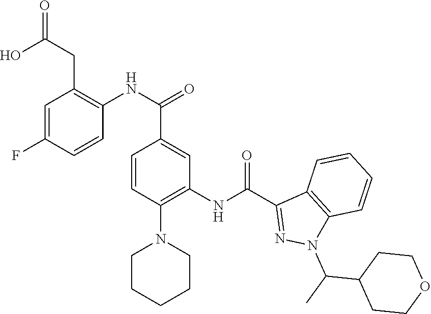 [SMILES: CC(C1CCOCC1)n1nc(C(=O)Nc2cc(C(=O)Nc3ccc(F)cc3CC(=O)O)ccc2N2CCCCC2)c2ccccc21] | ¹H NMR (400 MHz, DMSO-d6) δ: 12.40 (br s, 1 H), 10.09 (s, 1 H), 10.04 (br s, 1 H), 8.87 − 9.08 (m, 1 H), 8.24 − 8.33 (m, 1 H), 7.94 (d, J = 8.87 Hz, 1 H), 7.71 (dd, J = 8.36, 2.03 Hz, 1 H), 7.49-7.54 (m, 1 H), 7.43 (dd, J = 8.87, 5.58 Hz, 1 H), 7.33 − 7.39 (m, 2 H), 7.22 (dd, J = 9.76, 2.91 Hz, 1 H), 7.16 (td, J = 8.49, 3.04 Hz, 1 H), 4.77 − 4.92 (m, 1 H), 3.90 − 3.98 (m, 1 H), 3.70 − 3.79 (m, 1 H), 3.66 (s, 2 H), 3.20 − 3.30 (m, 1 H), 3.08- 3.17 (m, 1 H), 2.85 − 2.96 (m, 4 H), 2.16 − 2.27 (m, 1 H), 1.75 − 1.91 (m, 5 H), 1.61 − 1.70 (m, 2 H), 1.59 |
| | | | (d, J = 6.59 Hz, 3 H), 1.33 − |
| | | | 1.47 (m, 1 H), 1.17 − 1.32 |
| | | | (m, 1 H), 0.83 − 0.96 (m, 1 H). |
| | | | LC-MS: Rₜ = 1.37 min; MS |
| | | | (ESI+): m/z = 628.1 [M + H]⁺. |
| | | | prep-HPLC (Method |
| | | | described herein, Water |
| | | | (Acid): MeCN, gradient E) |

The following examples 103 and 135 were prepared in analogy to example 96 and converted to TFA salt after H-PLC purification, by dissolving the compound in DCM, adding few drops of TFA, evaporating the mixture, and drying the compound under the high vacuum; or crashed out as HCl salt in the reaction mixture, washed with DCM and dried under the high vacuum.

| Compound | | | |
|---|---|---|---|
| # | Name | Structure | Analytical Data |
| 103 | 2,2,2-trifluoroacetic acid--2-(5-fluoro-2- (4-(piperidin-1-yl)- 3-(1-(piperidin-4- ylmethyl)-1H- indazole-3- carboxamido) benzamido) phenyl)acetic acid (2/1) | 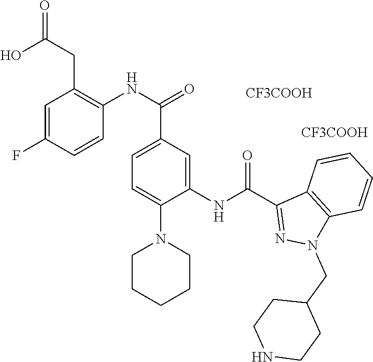 [SMILES: O=C(O)Cc1cc(F)ccc1NC(=O)c1ccc(N2CCCCC2)c(NC(=O)c2nn(CC3CCNCC3)c3ccccc23)c1 ; "CF3COOH" ; "CF3COOH"] | ¹H NMR (400 MHz, DMSO-d6) δ: 12.71 (s, 1H), 10.00 (s, 1H), 9.97 (s, 1H), 8.97-9.03 (m, 1H), 8.41-8.51 (m, 1H), 8.26-8.33 (m, IH), 8.07-8.22 (m, 1H), 7.89-7.95 (m, 1H), 7.72 (dd, J = 8.36, 2.03 Hz, 1H), 7.53-7.58 (m, 1H), 7.33-7.44 (m, 3 H), 7.23 (dd, J = 9.63, 3.04 Hz, 1H), 7.16 (td, J = 8.49, 3.04 Hz, 1H), 4.55 (d, J = 6.34 Hz, 2H), 3.67 (s, 2H), 3.20-3.32 (m, 2H), 2.90-2.97 (m, 4H), 2.75-2.89 (m, 2H), 2.19-2.31 (m, 1H), 1.77-1.88 (m, 6H), 1.61-1.72 (m, 2H), 1.39-1.54 (m, 2H). ¹⁹F NMR (377 MHz, DMSO-d6) δ: −74.41 (s, 6F), −119.20 − −116.07 (m, 1 F). UPLC-MS : Rₜ = 0.80 min; MS (ESI+): m/z = 614.0 [M + H]⁺. prep-HPLC (Method described herein, Water (Acid): MeCN, gradient C) |
| 135 | 2-(5-fluoro-2-(4- (piperidin-1-yl)-3- (2-(2,2,2- trifluoroethyl)-2H- indazole-3- carboxamido) benzamido) phenyl)acetic acid hydrochloride | 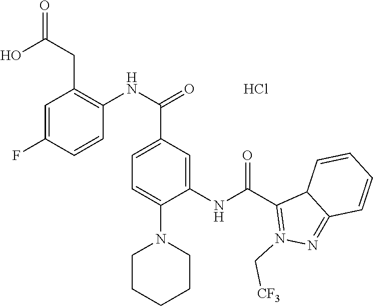 | ¹H NMR (400 MHz, DMSO-d₆) δ: 10.01 (s, 1 H), 9.90 (br s, 1 H), 8.65 − 8.73 (m, 1 H), 8.19 − 8.26 (m, 1 H), 7.88 − 7.93 (m, 1 H), 7.79 − 7.87 (m, 1 H), 7.38 - 7.54 (m, 4 H), 7.19 − 7.25 (m, 1 H), 7.16 (td, J = 8.62, 3.04 Hz, 1 H), 5.83 − 5.97 (m, 2 H), 3.67 (s, 2 H), 2.88 − 2.99 (m, 4 H), 1.58 − 1.70 (m, 4 H), 1.46 − 1.58 (m, 2 H). LC-MS: Rₜ = 1.43 min; MS (ESI+): m/z = 598.3 [M + H]⁺. |

### Example 22: 2,2,2-trifluoroacetic acid-2-(2-(3-(1-(2-aminoethyl)-1H-indazole-3-carboxamido)-4-(piperidin-1-yl)benzamido)-S-fluorophenyl)acetic acid

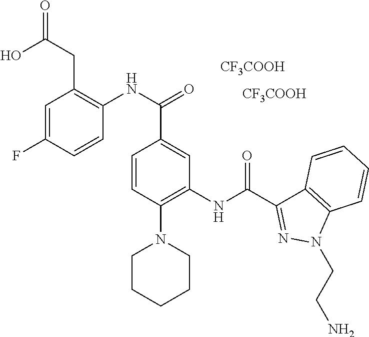 [SMILES: NCCn1nc(C(=O)Nc2cc(C(=O)Nc3ccc(F)cc3CC(=O)O)ccc2N2CCCCC2)c2ccccc21 ; "CF3COOH" ; "CF3COOH"]

Intermediate 10e (110 mg, 1 Eq, 154 μmol) was dissolved in DCM (2 mL) and TFA (877 mg, 593 μL, 50 Eq, 7.69 mmol) was added dropwise. The resulting mixture was stirred at 25° C. for 1 hour. The mixture was evaporated to dryness. The remaining crude was purified by prep-HPLC (Method described herein, Water (Basic):MeCN, gradient C). Product was dissolved in DCM and a few drops of TFA were added. The mixture was evaporated to dryness and dried under the high vacuum to give the title compound (32 mg, 26%).

¹H NMR (400 MHz, DMSO-d₆) δ: 12.41 (br s, 1H), 9.91-10.01 (m, 2H), 8.94-9.02 (m, 1H), 8.29-8.36 (m, 1H), 7.92-8.06 (m, 3H), 7.81-7.87 (m, 1H), 7.73 (dd, J=8.36, 2.03 Hz, 1H), 7.55-7.63 (m, 1H), 7.35-7.46 (m, 3H), 7.23 (dd, J=9.63, 3.04 Hz, 1H), 7.16 (td, J=8.55, 3.17 Hz, 1H), 4.76-4.82 (m, 2H), 3.67 (s, 2H), 3.47-3.54 (m, 2H), 2.86-2.99 (m, 4H), 1.77-1.88 (m, 4H), 1.60-1.72 (m, 2H).

¹⁹F NMR (377 MHz, DMSO-d₆) δ: −74.67 (s, 6F), −117.59-117.43 (m, 1F).

LC-MS: Rₜ=0.98 min; MS (ESI+): m/z=559.8 [M+H]⁺.

The following examples were prepared in a methodology analogous to the Example 22 with suitable reagents, precursors and starting materials:

| Compound# | Name | Structure | Analytical Data |
|---|---|---|---|
| 102 | 2-(2-(3-(1- (cyanomethyl)-1H- indazole-3- carboxamido)-4- (piperidin-1- yl)benzamido)-5- fluorophenyl)acetic acid | 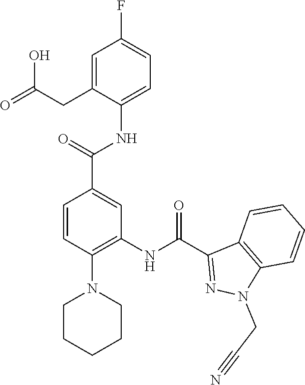 [SMILES: N#CCn1nc(C(=O)Nc2cc(C(=O)Nc3ccc(F)cc3CC(=O)O)ccc2N2CCCCC2)c2ccccc21] | ¹H NMR (400 MHz, DMSO-d₆) δ 10.05 (s, 1H), 9.98 (s, 1H), 8.97 (d, J = 2.0 Hz, 1H), 8.33 (d, ) = 8.1 Hz, 1H), 7.94 (d, J = 8.6 Hz, 1H), 7.73 (dd, J = 8.4, 2.0 Hz, 1H), 7.64 (ddd, J = 8.4, 7.0, 1.0 Hz, 1H), 7.51- 7.34 (m, 3H), 7.27- 7.11 (m, 2H), 6.00 (s, 2H), 3.67 (s, 2H), 2.96-2.87 (m, 4H), 1.93-1.80 (m, 4H), 1.62 (br s, 2H) UPLC-MS (method 5): Rₜ = 0.81 min. MS (ESI+): m/z = 555.8 [M + H]⁺. |
| 117 | 2-(2-(3-(4-cyclopropyl- 1-(2,2,2-trifluoroethyl)- 1H-pyrazole-3- carboxamido)-4- (piperidin-1- yl)benzamido)-5- fluorophenyl)acetic acid | 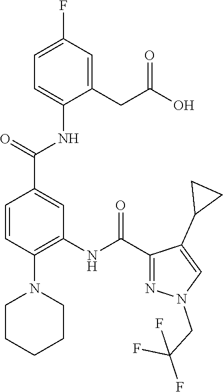 [SMILES: O=C(O)Cc1cc(F)ccc1NC(=O)c1ccc(N2CCCCC2)c(NC(=O)c2nn(CC(F)(F)F)cc2C2CC2)c1] | ¹H NMR (400 MHz, DMSO-d6) δ: 12.39 (br s, 1 H), 9.99 (br s, 1 H), 9.83 (s, 1 H), 8.87-8.98 (m, 1 H), 7.62-7.74 (m, 2 H), 7.37-7.43 (m, 1 H), 7.30-7.34 (m, 1 H), 7.18-7.23 (m, 1 H), 7.11-7.18 (m, 1 H), 5.11-5.22 (m, 2 H), 3.66 (s, 2 H), 2.81- 2.90 (m, 4 H), 2.39- 2.47 (m, 1 H), 1.71- 1.83 (m, 4 H), 1.57 (br s, 2 H), 0.90-0.97 (m, 2 H), 0.51-0.60 (m, 2 H). LC-MS: Rₜ = 1.3 min; MS (ESI+): m/z = 588.1 [M + H]⁺. prep-HPLC (Method described herein, Water (Acid): |
| | | | MeCN, gradient E) |
| 122 | 2-(5-fluoro-2-(4- (piperidin-1-yl)-3-(1- (prop-2-yn-1-yl)-1H- indazole-3- carboxamido)benzamido) phenyl)acetic acid | 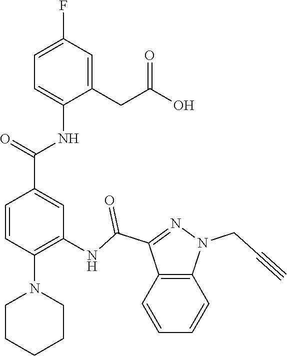 [SMILES: C#CCn1nc(C(=O)Nc2cc(C(=O)Nc3ccc(F)cc3CC(=O)O)ccc2N2CCCCC2)c2ccccc21] | ¹H NMR (400 MHz, DMSO-d6) δ: 12.42 (br s, 1 H), 9.97- 10.10 (m, 2 H), 8.93- 9.01 (m, 1 H), 8.26- 8.34 (m, 1 H), 7.88- 7.93 (m, 1 H), 7.72 (dd, J = 8.36, 2.03 Hz, 1 H), 7.58 (ddd, J = 8.43, 7.03, 1.01 Hz, 1 H), 7.38-7.46 (m, 2 H), 7.36 (d, J = 8.36 Hz, 1 H), 7.22 (dd, J = 9.63, 3.04 Hz, 1 H), 7.16 (td, J = 8.49, 3.04 Hz, 1 H), 5.53 (d, J = 2.53 Hz, 2 H), 3.67 (s, 2 H), 3.55 (t, J = 2.53 Hz, 1 H), 2.86- 2.98 (m, 4 H), 1.80- 1.93 (m, 4 H), 1.59- 1.71 (m, 2 H). |
| | | | LC-MS: Rₜ = 1.40 min; |
| | | | MS (ESI+): m/z = |
| | | | 554.3 [M + H]⁺. |
| | | | prep-HPLC (Method |
| | | | described herein, |
| | | | Water (Acid): |
| | | | MeCN, gradient D) |
| 127 | 2-(2-(3-(1-((1- cyanocyclopropyl) methyl)-1H-indazole-3- carboxamido)-4- (piperidin-1- yl)benzamido)-5- fluorophenyl)acetic acid | 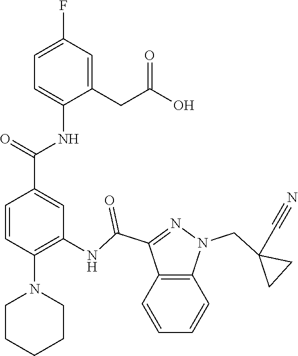 [SMILES: N#CC1(Cn2nc(C(=O)Nc3cc(C(=O)Nc4ccc(F)cc4CC(=O)O)ccc3N3CCCCC3)c3ccccc32)CC1] | ¹H NMR (400 MHz, DMSO-d6) δ: 12.43 (br s, 1 H), 10.03 (s, 1 H), 9.94 (s, 1 H), 8.95- 9.06 (m, 1 H), 8.29- 8.38 (m, 1 H), 7.96- 8.04 (m, 1 H), 7.70- 7.76 (m, 1 H), 7.54- 7.63 (m, 1 H), 7.35- 7.45 (m, 3 H), 7.20- 7.25 (m, 1 H), 7.12- 7.19 (m, 1 H), 4.81 (s, 2 H), 3.67 (s, 2 H), 2.87-2.96 (m, 4 H), 1.81-1.90 (m, 4 H), 1.63 (br s, 2 H), 1.44 (s, 4 H). LC-MS: Rₜ = 1.35 min; MS (ESI+): m/z = 595.4 [M + H]⁺. prep-HPLC (Method described herein, |
| | | | Water (Acid): |
| | | | MeCN, gradient D) |
| 128 | 2-(5-fluoro-2-(4- (piperidin-1-yl)-3-(1- (2,2,2-trifluoroethyl)- 1H-indole-3- carboxamido)benzamido) phenyl)acetic acid | 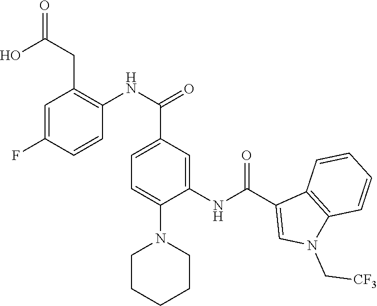 [SMILES: O=C(O)Cc1cc(F)ccc1NC(=O)c1ccc(N2CCCCC2)c(NC(=O)c2cn(CC(F)(F)F)c3ccccc23)c1] | ¹H NMR (400 MHz, DMSO-d6) δ: 12.42 (s, 1 H), 9.98 (s, 1 H), 9.22 (s, 1 H), 8.70 (d, J = 2.03 Hz, 1 H), 8.30 (s, 1 H), 8.22-8.27 (m, 1 H), 7.75-7.81 (m, 1 H), 7.72 (dd, J = 8.36, 2.28 Hz, 1 H), 7.39-7.45 (m, 1 H), 7.28-7.39 (m, 3 H), 7.19-7.23 (m, 1 H), 7.12-7.18 (m, 1 H), 5.33-5.47 (m, 2 H), 3.66 (m, 2 H), 2.85- 2.96 (m, 4 H), 1.67- 1.79 (m, 4 H), 1.49- 1.64 (m, 2 H). ¹⁹F NMR (377 MHz, |
| | | | DMSO-d6) δ: −70.42 |
| | | | (t, J = 9.73 Hz, 3F), |
| | | | −117.58-−117.70 (m, |
| | | | 1F). |
| | | | LC-MS: Rₜ = 1.40 min; |
| | | | MS (ESI+): m/z = |
| | | | 597.3 [M + H]⁺. |
| | | | prep-HPLC (Method |
| | | | described herein, |
| | | | Water (Acid); |
| | | | MeCN, gradient D) |
| 130 | 2-(5-fluoro-2-(3-(2- methyl-1-(2,2,2- trifluoroethyl)-1H- indole-3-carboxamido)- 4-(piperidin-1- yl)benzamido)phenyl) acetic acid | 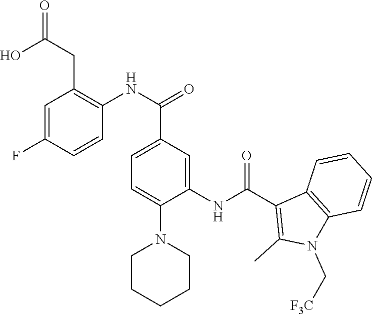 [SMILES: Cc1c(C(=O)Nc2cc(C(=O)Nc3ccc(F)cc3CC(=O)O)ccc2N2CCCCC2)c2ccccc2n1CC(F)(F)F] | ¹H NMR (400 MHz, DMSO-d6) δ: 12.43 (br s, 1 H), 10.05 (br s, 1 H), 9.38 (s, 1 H), 8.96-9.04 (m, 1 H), 8.02-8.12 (m, 1 H), 7.73-7.77 (m, 1 H), 7.69 (dd, J = 8.24, 2.15 Hz, 1 H), 7.40-7.46 (m, 2 H), 7.27-7.36 (m, 2 H), 7.19-7.24 (m, 1 H), 7.12-7.19 (m, 1 H), 5.31-5.41 (m, 2 H), 3.66 (s, 2 H), 2.85-2.90 (m, 4 H), 2.80 (s, 3 H), 1.59- 1.67 (m, 4 H), 1.46- 1.58 (m, 2 H). ¹⁹F NMR (377 MHz, |
| | | | DMSO-d6) δ: −69.06 |
| | | | (t, J = 9.16 Hz, 3 F), |
| | | | −117.59-−117.71 (m, |
| | | | 1 F). |
| | | | LC-MS: Rₜ = 1.45 min; |
| | | | MS (ESI+): m/z = |
| | | | 611.4 [M + H]⁺. |
| | | | prep-HPLC (Method |
| | | | described herein, |
| | | | Water (Acid) |
| | | | MeCN, gradient E) |
| 142 | 2-(5-fluoro-2-(3-(5- methyl-1-(2,2,2- trifluoroethyl)-1H- indazole-3- carboxamido)-4- (piperidin-1- yl)benzamido)phenyl) acetic acid | 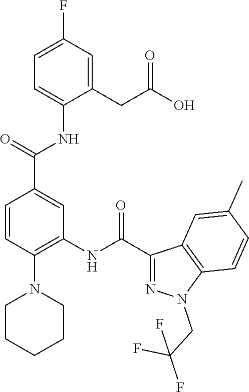 [SMILES: Cc1ccc2c(c1)c(C(=O)Nc1cc(C(=O)Nc3ccc(F)cc3CC(=O)O)ccc1N1CCCCC1)nn2CC(F)(F)F] | ¹H NMR (400 MHz, DMSO-d6) δ: 12.42 (s, 1 H), 10.03 (br s, 1 H), 9.98 (s, 1 H), 8.93- 9.02 (m, 1 H), 8.08- 8.13 (m, 1 H), 7.88 (d, J = 8.87 Hz, 1 H), 7.72 (dd, J = 8.36, 2.03 Hz, 1 H), 7.39-7.48 (m, 2 H), 7.37 (d, J = 8.36 Hz, 1 H), 7.22 (dd, J = 9.63, 3.04 Hz, 1 H), 7.16 (td, J = 8.55, 2.92 Hz, 1 H), 5.62 (g, J = 9.04 Hz, 2 H), 3.67 (s, 2 H), 2.83-2.99 (m, 4 H), 2.48 (br s, 3 H), 1.77-1.87 (m, 4 H), 1.52-1.70 (m, 2 H). LC-MS: Rt = 1.40 min; MS (ESI+): m/z = 612.1 [M + H]⁺. prep-HPLC (Method described herein, |
| | | | Water (Acid) |
| | | | MeCN, gradient F) |
| 144 | 2-(5-fluoro-2-(3-(7- methyl-1-(2,2,2- trifluoroethyl)-1H- indazole-3- carboxamido)-4- (piperidin-1- yl)benzamido)phenyl) acetic acid | 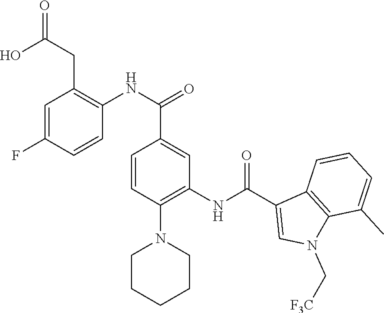 [SMILES: Cc1cccc2c(C(=O)Nc3cc(C(=O)Nc4ccc(F)cc4CC(=O)O)ccc3N3CCCCC3)cn(CC(F)(F)F)c12] | ¹H NMR (400 MHz, DMSO-d6) δ: 12.42 (br s, 1 H), 9.94- 10.07 (m, 2 H), 8.93- 9.05 (m, 1 H), 8.17- 8.29 (m, 1 H), 7.68- 7.77 (m, 1 H), 7.39- 7.45 (m, 1 H), 7.35- 7.39 (m, 1 H), 7.29- 7.39 (m, 2 H), 7.22 (dd, J = 9.63, 3.04 Hz, 1 H), 7.16 (td, J = 8.49, 3.04 Hz, 1 H), 5.58- 5.72 (m, 2 H), 3.66 (s, 2 H), 2.84-2.95 (m, 4 H), 2.75 (s, 3 H), 1.77- 1.86 (m, 4 H), 1.53- 1.68 (m, 2 H). ¹⁹F NMR (377 MHz, |
| | | | DMSO-d6) δ: −69.67 |
| | | | (t, J = 8.58 Hz, 3 F), |
| | | | −119.77-−116.15 (m, |
| | | | 1 F) |
| | | | LC-MS: Rt = 1.40 min; |
| | | | MS (ESI+): m/z = |
| | | | 612.1 [M + H] ⁺. |
| | | | prep-HPLC (Method |
| | | | described herein, |
| | | | Water (Acid); |
| | | | MeCN, gradient E) |
| 147 | 2-(2-(3-(1-(1- cyclopropylethyl)-1H- indazole-3- carboxamido)-4- (piperidin-1- yl)benzamido)-5- fluorophenyl)acetic acid | 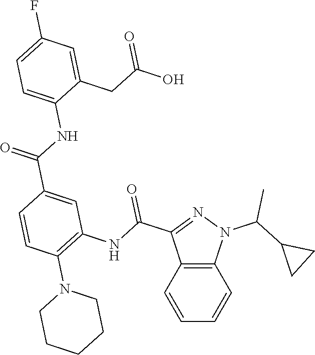 [SMILES: CC(C1CC1)n1nc(C(=O)Nc2cc(C(=O)Nc3ccc(F)cc3CC(=O)O)ccc2N2CCCCC2)c2ccccc21] | ¹H NMR (400 MHz, DMSO-d₆) δ: 12.42 (br s, 1 H), 10.15 (s, 1 H), 10.05 (br s, 1 H), 8.92-9.08 (m, 1 H), 8.28 (d, J = 8.11 Hz, 1 H), 7.85-7.92 (m, 1 H), 7.68-7.74 (m, 1 H), 7.47-7.53 (m, 1 H), 7.43 (dd, J = 8.87, 5.58 Hz, 1 H), 7.33- 7.39 (m, 2 H), 7.22 (dd, J = 9.63, 3.04 Hz, 1 H), 7.16 (td, J = 8.55, 2.91 Hz, 1 H), 4.36- 4.50 (m, 1 H), 3.66 (s, 2 H), 2.83-2.99 (m, 4 H), 1.79-1.90 (m, 4 H), 1.71 (d, J = 6.84 Hz, 3 H), 1.57- 1.68 (m, 2 H), 1.41- 1.50 (m, 1 H), 0.62- |
| | | | 0.74 (m, 1 H), 0.44- |
| | | | 0.54 (m, 1 H), 0.29- |
| | | | 0.44 (m, 2 H). |
| | | | UPLC-MS: Rₜ = |
| | | | 1.50 min; |
| | | | MS (ESI+): m/z = |
| | | | 584.6 [M + H]⁺. |
| | | | prep-HPLC (Method |
| | | | described herein, |
| | | | Water (Acid) |
| | | | MeCN, gradient F) |

### Example 23: 2,2,2-trifluoroacetic acid-2-(5-fluoro-2-(4-(piperidin-1-yl)-3-(1-(2-(pyrrolidin-1-yl)ethyl)-1H-indazole-3-carboxamido)benzamido)phenyl)acetic acid (2/1)

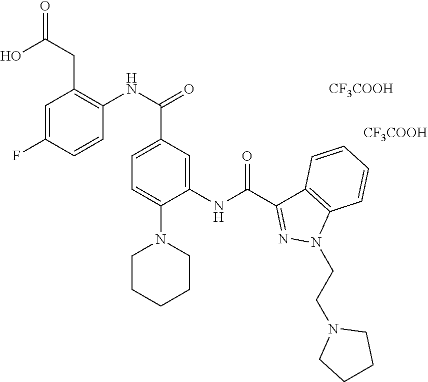 [SMILES: O=C(O)Cc1cc(F)ccc1NC(=O)c1ccc(N2CCCCC2)c(NC(=O)c2nn(CCN3CCCC3)c3ccccc23)c1 ; "CF3COOH" ; "CF3COOH"]

### Step 1: tert-butyl 2-(5-fluoro-2-(3-(J-(2-((methylsulfonyl)oxy)ethyl)-1H-indazole-3-carboxamido)-4-(piperidin-1-yl)benzamido)phenyl)acetate

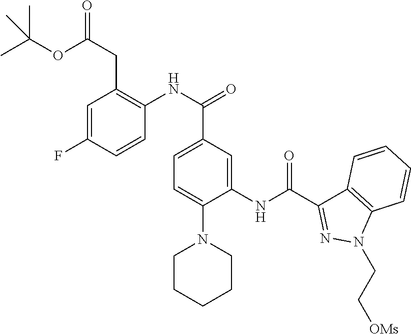 [SMILES: CC(C)(C)OC(=O)Cc1cc(F)ccc1NC(=O)c1ccc(N2CCCCC2)c(NC(=O)c2nn(CCOS(C)(=O)=O)c3ccccc23)c1]

Intermediate 10k (110 mg, 1 Eq, 179 μmol) was dissolved in DCM (2 mL) and cooled in an ice-bath. TEA (54.2 mg, 74.7 μL, 3 Eq, 536 μmol) and MsCl (61.4 mg, 41.8 μL, 3 Eq, 536 μmol) were added. Mixture is stirred for 30 min at 0° C., then 2 h at 25° C. Mixture is evaporated, EtOAc added, washed with water and brine. The obtained crude product is used in the next step without further purification.

UPLC-MS (method 3): Rₜ=1.51 min; MS (ESI+): m/z=694.8 [M+H]⁺.

### Step 2: tert-butyl 2-(S-fluoro-2-(4-(piperidin-1-yl)-3-(J-(2-(pyrrolidin-1-yl)ethyl)-1H-indazole-3-carboxamido)benzamido)phenyl)acetate

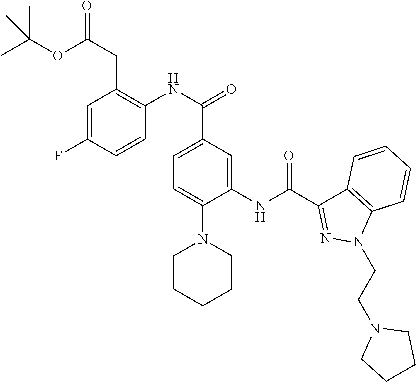 [SMILES: CC(C)(C)OC(=O)Cc1cc(F)ccc1NC(=O)c1ccc(N2CCCCC2)c(NC(=O)c2nn(CCN3CCCC3)c3ccccc23)c1]

Tert-butyl 2-(5-fluoro-2-(3-(1-(2-((methylsulfonyl)oxy)ethyl)-1H-indazole-3-carboxamido)-4-(piperidin-1-yl)benzamido)phenyl)acetate obtained in Step 1 (123 mg, 1 Eq, 177 μmol) was dissolved in acetonitrile (2.5 mL), then TEA (53.8 mg, 74.1 μL, 3 Eq, 532 μmol) and pyrrolidine (37.8 mg, 43.7 μL, 3 Eq, 532 μmol) were added and mixture stirred at 50° C. for 48 hours. Mixture is concentrated and the obtained crude was purified using Biotage flash silica gel chromatography (Sfaer Silica 25 g, DCM to DCM/MeOH=9/1), to give 94 mg of beige solid (790%).

UPLC-MS: Rₜ=1.67 min; MS (ESI+): m/z=670.1 [M+H]⁺.

### Step 3: 2,2,2-trifluoroacetic acid-2-(S-fluoro-2-(4-(piperidin-yl)-3-(1-(2-(pyrrolidin-1-yl)ethyl)-1H-indazole-3-carboxamido)benzamido)phenyl)acetic acid (24)

Tert-butyl 2-(5-fluoro-2-(4-(piperidin-1-yl)-3-(1-(2-(pyrrolidin-1-yl)ethyl)-1H-indazole-3-carboxamido)benzamido)phenyl)acetate obtained in Step 2 (94 mg, 1 Eq, 140 μmol) was dissolved in DCM (1 mL) and HCl in dioxane (128 mg, 0.88 mL, 4 molar, 25 Eq, 3.51 mmol) was added. Reaction mixture was stirred at rt for 1 h. Solvents were evaporated to dryness and the obtained crude was purified via prep-HPLC (Method described herein, Water (Basic): MeCN, gradient C). The obtained white powder showed poor solubility and was therefore converted to TFA salt by suspending it in DCM and adding couple of drops of TFA. Solvents were evaporated and the product was dried under the high vacuum overnight to give 22.6 mg of the title compound (19%).

¹H NMR (400 MHz, DMSO-d₆) δ: 12.33 (br s, 1H), 9.98 (s, 1H), 9.93 (s, 1H), 9.78 (br s, 1H), 8.96-9.03 (m, 1H), 8.30-8.37 (m, 1H), 7.95 (d, J=8.62 Hz, 1H), 7.71-7.77 (m, 1H), 7.58-7.65 (m, 1H), 7.35-7.46 (m, 3H), 7.20-7.25 (m, 1H), 7.16 (td, J=8.62, 3.04 Hz, 1H), 4.93-5.03 (m, 2H), 3.80-3.89 (m, 2H), 3.67 (s, 2H), 3.53-3.64 (m, 2H), 3.11-3.27 (m, 2H), 2.87-2.98 (m, 4H), 1.98-2.09 (m, 2H), 1.78-1.91 (m, 6H), 1.60-1.71 (m, 2H).

¹⁹F NMR (377 MHz, DMSO-d₆) δ: −74.83 (s, 6 F), −118.17-116.86 (m, 1 F).

UPLC-MS (method 3): R₅=1.67 min: MS (ESI+): m/z=613.8 [M+H]⁺.

### Example 24: 4-(2-(3-((5-((2-(carboxymethyl)-4-fluorophenyl)carbamoyl)-2-(piperidin-1-yl)phenyl)carbamoyl)-1H-indazol-1-yl)ethyl)morpholin-4-ium 2,2,2-trifluoroacetate

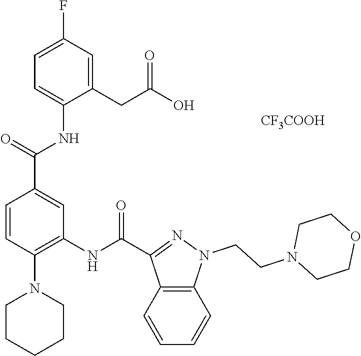 [SMILES: O=C(O)Cc1cc(F)ccc1NC(=O)c1ccc(N2CCCCC2)c(NC(=O)c2nn(CCN3CCOCC3)c3ccccc23)c1 ; "CF3COOH"]

### Step 1: tert-butyl 2-(S-fluoro-2-(3-(1-(2-morpholinoethyl)-1H-indazole-3-carboxamido)-4-(piperidin-1-yl)benzamido)phenyl)acetate

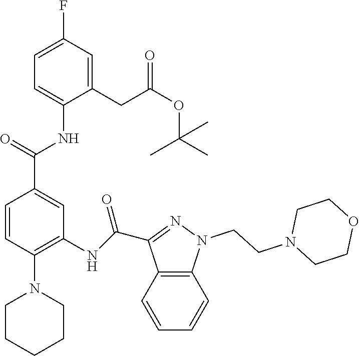 [SMILES: CC(C)(C)OC(=O)Cc1cc(F)ccc1NC(=O)c1ccc(N2CCCCC2)c(NC(=O)c2nn(CCN3CCOCC3)c3ccccc23)c1]

A mixture of tert-butyl 2-(5-fluoro-2-(3-(1-(2-((methylsulfonyl)oxy)ethyl)-1H-indazole-3-carboxamido)-4-(piperidin-1-yl)benzamido)phenyl)acetate (Step 1, Example 101) (79 mg, 1 Eq, 0.11 mmol), morpholine (30 mg, 3 Eq, 0.34 mmol), Et₃N (35 mg, 48 μL, 3 Eq, 0.34 mmol) and acetonitrile (2 mL) was stirred 80° C. for 18 hours. The mixture was evaporated to dryness and purified via prep-HPLC (Method described herein, Water (Acidic): MeCN, gradient E) to give the title compound (19 mg, 24%).

¹H NMR (400 MHz, DMSO-d₆) δ: 10.04 (s, 1H), 9.91 (s, 1H), 8.96-9.04 (m, 1H), 8.21-8.31 (m, 1H), 7.87-7.94 (m, 1H), 7.68-7.76 (m, 1H), 7.49-7.56 (m, 1H), 7.33-7.44 (m, 3H), 7.12-7.24 (m, 2H), 4.64-4.73 (m, 2H), 3.67 (s, 2H), 3.47-3.56 (m, 4H), 2.87-2.97 (m, 6H), 2.46-2.53 (m, 4H), 1.81-1.89 (m, 4H), 1.58-1.70 (m, 2H), 1.29 (s, 9H).

UPLC-MS: Rₜ=1.37 min; MS (ESI+): m/z=686.0 [M+H]⁺.

### Step 2: 4-(2-(3-((5-((2-(carboxymethyl)-4-fluorophenyl)carbamoyl)-2-(piperidin-1-yl)phenyl)carbamoyl)-1H-indazol-1-yl)ethyl)morpholin-4-ium 2,2,2-trifluoroacetate

A mixture of tert-butyl 2-(5-fluoro-2-(3-(1-(2-morpholinoethyl)-1H-indazole-3-carboxamido)-4-(piperidin-1-yl)benzamido)phenyl)acetate obtained in Step 1 (19 mg, 1 Eq, 28 μmol) in DCM (2 mL) and HCl in dioxane (25 mg, 0.17 mL, 4 molar, 25 Eq, 0.69 mmol) was stirred at 25° C. for 2 hour. The mixture was evaporated to dryness and obtained crude was purified via prep-HPLC (Method described herein, Water (Acidic): MeCN, gradient C). The obtained white powder showed poor solubility and was therefore converted to TFA salt by suspending it in DCM and adding couple of drops of TFA. Solvents were evaporated and the product was dried under the high vacuum overnight to give 10 mg of the title compound (49%).

¹H NMR (400 MHz, DMSO-d₆) δ: 12.42 (br s, 1H), 10.22 (br s, 1H), 9.97 (s, 1H), 9.91 (s, 1H), 8.93-9.02 (m, 1H), 8.33 (d, 0.1=8.36 Hz, 1H), 7.94 (d, J=8.62 Hz, 1H), 7.70-7.79 (m, 1H), 7.58-7.67 (m, 1H), 7.36-7.47 (m, 3H), 7.13-7.26 (m, 2H), 4.93-5.06 (m, 2H), 3.67 (s, 2H), 3.12-4.59 (m, 10H), 2.87-2.98 (m, 4H), 1.75-1.91 (m, 4H), 1.58-1.71 (m, 2H).

¹⁹F NMR (377 MHz, DMSO-d₆) δ: −74.51 (s, 3 F), −118.95-116.29 (m, 1 F).

LC-MS: Rₜ=1.05 min, MS (ESI+): m/z=629.4 [M+H]⁺.

### Example 25: 2-(5-fluoro-2-(4-(piperidin-1-yl)-3-((1-(2,2,2-trifluoroethyl)-1H-indazole)-3-sulfonamido)benzamido)phenyl)acetic acid

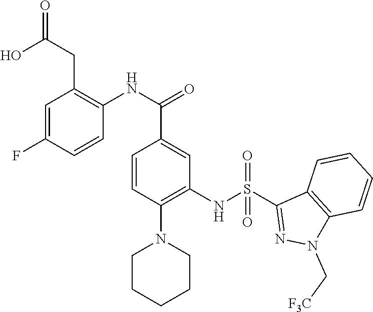 [SMILES: O=C(O)Cc1cc(F)ccc1NC(=O)c1ccc(N2CCCCC2)c(NS(=O)(=O)c2nn(CC(F)(F)F)c3ccccc23)c1]

### Step 1: 3-iodo-1-(2,2,2-trifluoroethyl)-1H-indazole

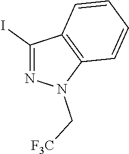 [SMILES: FC(F)(F)Cn1nc(I)c2ccccc21]

To a solution of 3-iodo-1H-indazole (3 g, 12.29 mmol, 1 eq) in THF (45 mL) was added NaH (737.61 mg, 18.44 mmol, 60%/6 purity, 1.5 eq) at 0° C. The mixture was stirred at 25° C. for 15 min. 2,2,2-trifluoroethyl trifluoromethanesulfonate (3.71 g, 15.98 mmol, 1.3 eq) was added dropwise at 0° C. The mixture was stirred at 25° C. for 16 h. The mixture was poured into ice-water (50 mL). The aqueous phase was extracted with ethyl acetate (50 mL×2). The combined organic phase was washed with brine (50 mL), dried with anhydrous Na₂SO₄, filtered, and concentrated in vacuum. The residue was purified by flash silica gel chromatography (40 g SepaFlash® Silica Flash Column, Eluent of 5-15% Ethyl acetate/Petroleum ether gradient @50 mL/min) to give the title compound (2.7 g, 8.28 mmol, 67.36% yield) as white solid.

¹H NMR (400 MHz, DMSO-d₆) δ=7.82 (dd, J=8.5 Hz, 1H), 7.59-7.54 (m, 1H), 7.48 (d, J=8.1 Hz, 1H), 7.33-7.27 (m, 1H), 5.51 (q, J=9.1 Hz, 2H).

LCMS-J; MS (ESIpos): m/z=327.0 [M+H]⁺.

### Step 2: 3-((4-methoxybenzyl)thio)-1-(2,2,2-trifluoroethyl)-1H-indazole

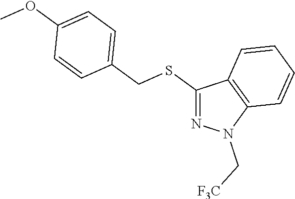 [SMILES: COc1ccc(CSc2nn(CC(F)(F)F)c3ccccc23)cc1]

To a solution of (4-methoxyphenyl)methanethiol (832.50 mg, 5.40 mmol, 1.1 eq) and 3-iodo-1-(2,2,2-trifluoroethyl)-1H-indazole (1.6 g, 4.91 mmol, 1 eq) in dioxane (15 mL) were added Pd₂(dba)₃ (224.68 mg, 245.36 μmol, 0.05 eq), DIPEA (1.27 g, 9.81 mmol, 1.71 mL, 2 eq) and Xantphos (141.97 mg, 245.36 μmol, 0.05 eq) under N₂. The mixture was stirred at 100° C. for 16 hr under N₂. The mixture was filtered, and the filtrate was concentrated under vacuum. The residue was purified by flash silica gel chromatography (40 g SepaFlash® Silica Flash Column, Eluent of 0-15% Ethyl acetate/Petroleum ether gradient @60 mL/min) to give the title compound (1.5 g, 4.26 mmol, 86.75% yield) as yellow oil.

¹H NMR (400 MHz, DMSO-d₆) δ=7.78 (d, J=8.6 Hz, 1H), 7.61 (d, J=8.1 Hz, 1H), 7.52-7.46 (m, 1H), 7.27-7.17 (m, 3H), 6.83-6.77 (m, 2H), 5.45 (q, J=9.1 Hz, 2H), 4.30 (s, 2H), 3.69 (s, 3H).

LCMS-A; MS (ESIpos): m/z=353.1 [M+H]⁺.

### Step 3: 1-(2,2,2-trifluoroethyl)-1H-indazole-3-sulfonyl chloride

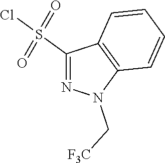 [SMILES: O=S(=O)(Cl)c1nn(CC(F)(F)F)c2ccccc12]

To a solution of 3-((4-methoxybenzyl)thio)-1-(2,2,2-trifluoroethyl)-1H-indazole (0.5 g, 1.42 mmol, 1 eq) in DCM (7 mL), AcOH (1 mL) and H₂O (2 mL) was added a solution of 1,3-dichloro-5,5-dimethyl-imidazolidine-2,4-dione (838.68 mg, 4.26 mmol, 3 eq) in DCM (4 mL) slowly at 0° C. The mixture was stirred at 0° C. for 1 hr. The mixture was poured into ice-water (50 mL). The aqueous phase was extracted with DCM (30 mL×2). The combined organic phase was washed with water (20 mL), dried with anhydrous Na₂SO₄, filtered, and concentrated in vacuum. The residue was purified by flash silica gel chromatography (25 g SepaFlash® Silica Flash Column, Eluent of 10-30% Ethylacetate/Petroleum ether gradient @60 mL/min) to give the title compound (0.4 g, 937.49 μmol, 66.07% yield, 70% purity) as yellow solid.

LCMS-AI; MS (ESIpos): m/z=347.9[M-C₁+piperidine]*.

### Step 4: tert-butyl 2-(S-fluoro-2-(4-(piperidin-1-yl)-3-((1-(2,2,2-trifluoroethyl)-1H-indazole)-3-sulfonamido)benzamido)phenyl)acetate

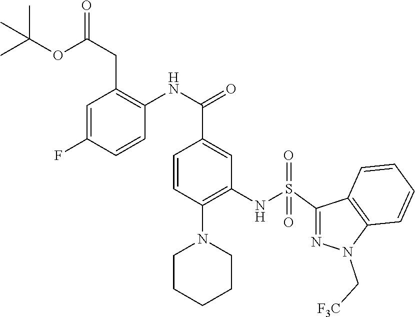 [SMILES: CC(C)(C)OC(=O)Cc1cc(F)ccc1NC(=O)c1ccc(N2CCCCC2)c(NS(=O)(=O)c2nn(CC(F)(F)F)c3ccccc23)c1]

A solution of 1-(2,2,2-trifluoroethyl)-1H-indazole-3-sulfonyl chloride (125.75 mg, 421.04 μmol, 1 0.5 eq) and tert-butyl 2-(2-(3-amino-4-(piperidin-1-yl)benzamido)-5-fluorophenyl)acetate (120 mg, 280.69 μmol, 1 eq) in Py (3 mL) was stirred at 25° C. for 16 h. The mixture was concentrated under vacuum. The residue was poured into water (30 mL). The aqueous phase was extracted with ethyl acetate (30 mL×2). The combined organic phase was washed with brine (20 mL), dried with anhydrous Na₂SO₄, filtered, and concentrated in vacuum. The residue was purified by flash silica gel chromatography (20 g SepaFlash® Silica Flash Column, Eluent of 20-40% Ethyl acetate/Petroleum ether gradient @40 mL/min) to give the title compound (0.18 g, 182.68 μmol, 65.08% yield, 70% purity) as brown oil.

LCMS-P; MS (ESIpos): m/z=690.2 [M+H]⁺.

### Step 5: 2-(5-fluoro-2-(4-(piperidin-yl)-3-((1-(2,2,2-trifluoroethyl)-1H-indazole)-3-sulfonamido)benzamido)phenylacetic acid

To a solution of tert-butyl 2-(5-fluoro-2-(4-(piperidin-1-yl)-3-((1-(2,2,2-trifluoroethyl)-1H-indazole)-3-sulfonamido)benzamido)phenyl)acetate (0.18 g, 182.68 μmol, 70% purity, 1 eq) in DCM (2 mL) was added TFA (3.08 g, 27.01 mmol, 2 mL, 147.86 eq). The mixture was stirred at 25° C. for 0.5 h. The mixture was concentrated under vacuum. The residue was purified by prep-HPLC (column: Unisil 3-100 C18 Ultra 150*50 mm*3 um, mobile phase: [water(FA)-ACN]; B %: 43%-73%, 7 min) followed by lyophilization to give the title compound (49.1 mg, 75.94 μmol, 41.57% yield, 98% purity) as white solid.

¹H NMR (400 MHz, DMSO-d₆) δ=12.84-12.01 (m, 1H), 10.04 (br s, 1H), 9.65-9.29 (m, 1H), 7.94 (d, J=2.0 Hz, 1H), 7.91 (d, J=8.7 Hz, 1H), 7.83 (d, J=8.2 Hz, 1H), 7.74 (dd, J=1.8, 8.4 Hz, 1H), 7.59 (t, J=7.8 Hz, 1H), 7.43-7.33 (m, 2H), 7.19 (dd, J=2.9, 9.7 Hz, 1H), 7.13 (dt, J=3.1, 8.6 Hz, 1H), 7.08 (d, J=8.4 Hz, 1H), 5.59 (q, J=9.0 Hz, 2H), 3.61 (s, 2H), 2.55-2.52 (m, 4H), 1.35-1.21 (m, 6H).

LCMS-N; HPLC-BM; MS (ESIpos): m/z=634.3 [M+H]⁺.

### Example 26: 2-(2-(3-((1-(cyanomethyl)-1H-indazol-3-yl)carbamoyl)-4-(piperidin-1-yl)benzamido)-5-fluorophenyl)acetic acid

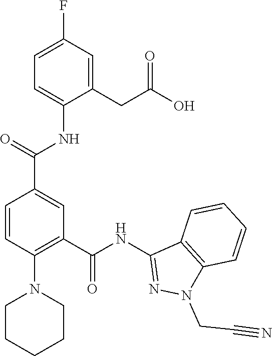 [SMILES: N#CCn1nc(NC(=O)c2cc(C(=O)Nc3ccc(F)cc3CC(=O)O)ccc2N2CCCCC2)c2ccccc21]

A mixture of intermediate 16a (50 mg, 1 Eq, 82 μmol), TFA (93 mg, 63 μL, 10 Eq, 0.82 mmol) and DCM (2 mL) was stirred at 25° C. for 1 hour. The mixture was evaporated to dryness. Crude was purified by prep-HPLC (Method described herein, Water (Acidic): MeCN, gradient D): to give 28 mg (62%) of the title compound.

¹H NMR (400 MHz, DMSO-d₆) δ: 12.42 (br s, 1H), 11.97 (s, 1H), 10.35 (br s, 1H), 8.40-8.48 (m, 1H), 8.03-8.13 (m, 2H), 7.74-7.82 (m, 1H), 7.51-7.57 (m, 1H), 7.38-7.46 (m, 2H), 7.11-7.27 (m, 3H), 5.77 (s, 2H), 3.64 (s, 2H), 3.05-3.15 (m, 4H), 1.68-1.79 (m, 4H), 1.50-1.65 (m, 2H).

LC-MS: Rₜ=1.10 min; MS (ESI+): m/z=555.1 [M+H]˜.

The following examples were prepared in a methodology analogous to the Example 26 with suitable reagents, precursors and starting materials:

| Compound # | Name | Structure | Analytical Data |
|---|---|---|---|
| 41 | 2-(5-fluoro-2-(4- (piperidin-1-yl)- 3-((1-(2,2,2- trifluoroethyl)- 1H-indazol-3- yl)carbamoyl) benzamido)phenyl) acetic acid | 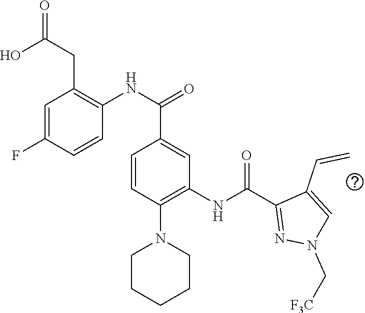 [SMILES: C=Cc1cn(CC(F)(F)F)nc1C(=O)Nc1cc(C(=O)Nc2ccc(F)cc2CC(=O)O)ccc1N1CCCCC1] | ¹H NMR (400 MHz, DMSO-d₆) δ = 12.04 (s, 1H), 11.20-10.48 (m, 1H), 8.47 (d, J = 2.1 Hz, 1H), 8.19- 8.02 (m, 2H), 7.77 (d, J = 8.4 Hz, 1H), 7.51 (q, J = 7.8 Hz, 2H), 7.40 (d, J = 8.6 Hz, 1H), 7.26-7.06 (m, 3H), 5.40 (q, J = 9.1 Hz, 2H), 3.60 (s, 2H), 3.16-3.05 (m, 4H), 1.83-1.64 (m, 4H), 1.60-1.41 (m, 2H). LCMS-A; HPLC- E; MS (ESIpos): |
| | | | m/z 598.2 |
| | | | [M + H]⁺. |
| 150 | 2-(5-fluoro-2-(4- (piperidin-1-yl)- 3-((1- ((tetrahydro-2H- pyran-4- yl)methyl)-1H- indazol-3- yl)carbamoyl) benzamido)phenyl) acetic acid | 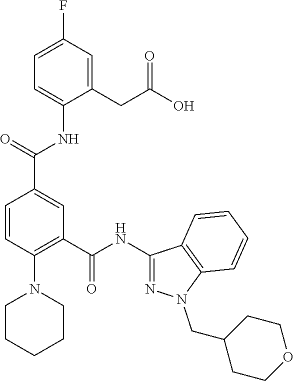 [SMILES: O=C(O)Cc1cc(F)ccc1NC(=O)c1ccc(N2CCCCC2)c(C(=O)Nc2nn(CC3CCOCC3)c3ccccc23)c1] | ¹H NMR (400 MHz, DMSO-d6) δ: 12.39 (br s, 1 H), 12.01 (s, 1 H), 10.15 (m, 1 H), 8.43-8.52 (m, 1 H), 8.01-8.13 (m, 2 H), 7.68 (d, J = 8.62 Hz, 1 H), 7.34-7.46 (m, 3 H), 7.21 (dd, J = 9.76, 2.91 Hz, 1 H), 7.08-7.18 (m, 2 H), 4.26 (d, J = 6.84 Hz, 2 H), 3.78-3.86 (m, 2 H), 3.65 (s, 2 H), 3.19-3.28 (m, 2 H), 3.03-3.16 (m, 4 H), 2.09-2.22 (m, 1 H), 1.70- 1.83 (m, 4 H), 1.52- 1.62 (m, 2 H), |
| | | | 1.26-1.47 (m, 4 H). |
| | | | UPLC-MS: Rₜ = |
| | | | 1.27 min; MS |
| | | | (ESI+): m/z = 614.8 |
| | | | [M + H]⁺. |
| | | | prep-HPLC |
| | | | (Method described |
| | | | herein, Water |
| | | | (Acidic); MeCN, |
| | | | gradient D) |

### Example 27: 2-(5-fluoro-2-(4-(piperidin-1-yl)-3-(3-(1-(2,2,2-trifluoroethyl)-1H-indazol-3-yl)ureido)benzamido)phenyl)acetic acid

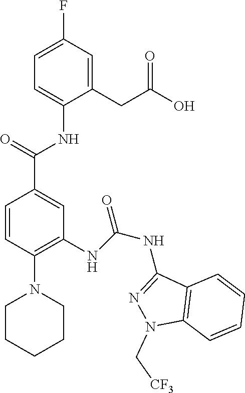 [SMILES: O=C(O)Cc1cc(F)ccc1NC(=O)c1ccc(N2CCCCC2)c(NC(=O)Nc2nn(CC(F)(F)F)c3ccccc23)c1]

To the solution of intermediate 18 (80 mg, 1 Eq, 0.12 mmol) in DCM (2 mL) was added HCl in dioxane (44 mg, 0.30 mL, 4 molar, 10 Eq, 1.2 mmol) and the resulting mixture was stirred at 25° C. for 2 hours. The solvent was removed under reduce pressure. The remaining crude material was purified by prep-HPLC (Method described herein, Water (Basic): MeCN, gradient C) to give 25 mg of the title compound (34%).

¹H NMR (400 MHz, DMSO-d₆) δ:10.81 (br s, 1H), 10.29 (s, 1H), 9.33 (br s, 1H), 8.58-8.61 (m, 1H), 8.08-8.18 (m, 1H), 7.66-7.79 (m, 2H), 7.46-7.59 (m, 2H), 7.05-7.26 (m, 4H), 5.26-5.40 (m, 2H), 3.57 (s, 2H), 2.80-2.90 (m, 4H), 1.67-1.77 (m, 4H), 1.50-1.61 (m, 2H).

LC-MS: Rₜ=1.30 min; MS (ESI+): m/z=613.1 [M+H].

### Example 28: 2-(4-(piperidin-1-yl)-3-(1-(2,2,2-trifluoroethyl)-1H-indazole-3-carboxamido)benzamido)benzoic acid

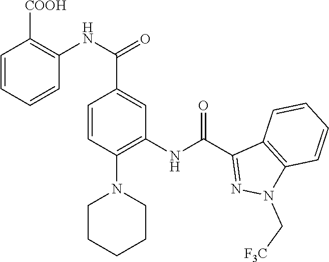 [SMILES: O=C(Nc1ccccc1C(=O)O)c1ccc(N2CCCCC2)c(NC(=O)c2nn(CC(F)(F)F)c3ccccc23)c1]

4-(piperidin-1-yl)-3-(1-(2,2,2-trifluoroethyl)-1H-indazole-3-carboxamido)benzoic acid (82 mg, 86% Wt, 1 Eq, 0.16 mmol) was dissolved in DMF (1.5 mL) then HATU (67 mg, 1.1 Eq, 0.18 mmol) followed by DIPEA (0.14 mL, 5 Eq, 0.8 mmol) were added, the reaction was stirred at rt for 1 h then 2-aminobenzoic acid (33 mg, 1.5 Eq, 0.24 mmol) was added and stirring was prolonged at rt for 16 hours. The reaction mixture was diluted with 0.5 mL of DMSO and 0.5 mL of CH₃CN/water 1:1, two drops of NaOH 1N were added and it was directly purified by preparative RP-HPLC, gradient C to obtain the desired material (61 mg, 67%).

¹H NMR (400 MHz, DMSO-d₆) δ 10.00 (s, 1H), 9.07 (d, J=2.0 Hz, 1H), 8.73 (dd, J=8.5, 0.9 Hz, 1H), 8.35-8.30 (m, 1H), 8.06 (dd, J=7.9, 1.5 Hz, 1H), 8.00 (d, J=8.6 Hz, 1H), 7.74 (dd, 1=8.2, 2.2 Hz, 1H), 7.66-7.54 (m, 2H), 7.47-7.39 (m, 2H), 7.18-7.12 (m, 1H), 5.66 (q, 1=9.0 Hz, 2H), 2.95-2.85 (m, 5H), 1.86-1.75 (m, 4H), 1.61 (br d, J=4.1 Hz, 2H).

LC-MS: Rₜ=1.58 min. MS (ESI+): m/z=566.4 [M+H]⁺.

### Example 29: 2-(3-fluoro-2-(4-(piperidin-1-yl)-3-(1-(2,2,2-trifluoroethyl)-1H-indazole-3-carboxamido)benzamido)phenyl)acetic acid

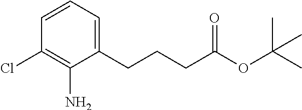 [SMILES: CC(C)(C)OC(=O)CCCc1cccc(Cl)c1N]

### Step 1: Tert-butyl 2-(3-fluoro-2-(4-(piperidin-1-yl)-3-((2,2,2-trifluoroethyl)-1H-indazole-3-carboxamido)benzamido)phenyl)acetate

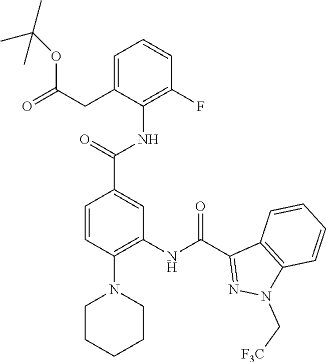 [SMILES: CC(C)(C)OC(=O)Cc1cccc(F)c1NC(=O)c1ccc(N2CCCCC2)c(NC(=O)c2nn(CC(F)(F)F)c3ccccc23)c1]

Tert-butyl 2-(2-amino-3-fluorophenyl)acetate (50 mg, 1. Eq, 0.22 mmol) dissolved in DCM (0.5 mL) and Py (1 mL), to this solution, a suspension of the 4-(piperidin-1-yl)-3-(1-(2,2,2-trifluoroethyl)-1H-indazole-3-carboxamido)benzoyl chloride (0.16 g, 86% Wt, 1.3 Eq, 0.29 mmol) in DCM (1 mL) was added, the reaction was stirred for 3 h then it was diluted with EtOAc and HCl 1N/ice, the organic phase was washed with brine, dried over sodium sulfate and reduced in vacuo. The residue was purified by automated flash column chromatography with a gradient 10-25-50% EtOAc in Hex to give the desired material (60 mg, 41%).

UPLC-MS: Rₜ=1.62 min. MS (ESI+): m/z=654.9 [M+H]⁺.

### Step 2: 2-(3-fluoro-2-(4-(piperidin-yl)-3-(1-(2,2,2-fluoroethyl)-3H-indazole-3-carboxamido)benzamido)phenyl)acetic acid

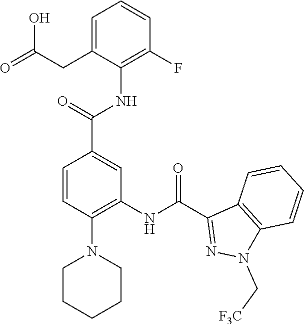 [SMILES: O=C(O)Cc1cccc(F)c1NC(=O)c1ccc(N2CCCCC2)c(NC(=O)c2nn(CC(F)(F)F)c3ccccc23)c1]

Tert-butyl 2-(3-fluoro-2-(4-(piperidin-1-yl)-3-(1-(2,2,2-trifluoroethyl)-1H-indazole-3-carboxamido)benzamido)phenyl)acetate (60 mg, 1 Eq, 92 μmol) was dissolved in DCM (3 mL) and TFA (1 mL) was added, the reaction was stirred 2h at rt then the solvent was removed in vacuo. The residue was purified with a by automated flash column chromatography with a gradient 10-20-40-70% eluent B (DCM MeOH 9:1) in eluent A (DCM) to give 50 mg of desired product (0.091 mmol, 91%).

¹H NMR (400 MHz, DMSO-d₆) δ 10.01 (s, 1H), 9.92 (s, 1H), 8.98 (d, J=2.0 Hz, 1H), 8.35-8.30 (m, 1H), 8.00 (d, J=8.6 Hz, 1H), 7.76 (dd, J=8.4, 2.0 Hz, 1H), 7.62 (ddd, J=8.4, 7.2, 1.1 Hz, 1H), 7.48-7.41 (m, 1H), 7.40-7.29 (m, 2H), 7.27-7.17 (m, 2H), 5.67 (q, J=9.2 Hz, 2H), 3.64 (s, 2H), 2.94-2.88 (m, 4H), 1.85-1.78 (m, 4H), 1.66-1.56 (m, 2H)

UPLC-MS: Rₜ=1.38 min. MS (EST+): m/z=598.7 [M+H]⁺.

### Example 30: 4-(4-(piperidin-1-yl)-3-(1-(2,2,2-trifluoroethyl)-1H-indazole-3-carboxamido)benzamido)nicotinic acid

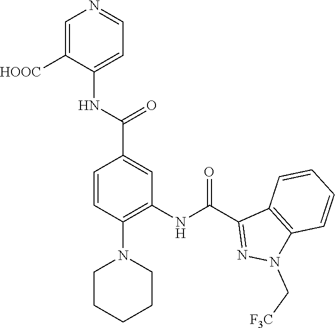 [SMILES: O=C(Nc1ccncc1C(=O)O)c1ccc(N2CCCCC2)c(NC(=O)c2nn(CC(F)(F)F)c3ccccc23)c1]

### Step 1: methyl 4-(4-(piperidin-1-yl)-3-(1-(2,2,2-trifluoroethyl)-1H-indazole-3-carboxamido)benzamido)nicotinate

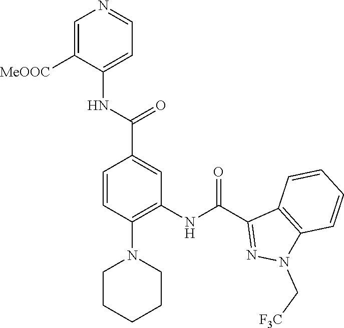 [SMILES: COC(=O)c1cnccc1NC(=O)c1ccc(N2CCCCC2)c(NC(=O)c2nn(CC(F)(F)F)c3ccccc23)c1]

The compound was synthesized following the coupling procedure described in coupling method described herein.

UPLC-MS (method 3): Rₜ=1.59 min. MS (ESI+): m/z=581.7 [M+H]⁺.

### Step 2: 4-(4-(piperidin-1-yl)-3-(1-(2,2,2-trifluoroethyl)-1H-indazole-3-carboxamido)benzamido)nicotinic acid

To a solution of methyl 4-(4-(piperidin-1-yl)-3-(1-(2,2,2-trifluoroethyl)-1H-indazole-3-carboxamido)benzamido)nicotinate (66 mg, 1 Eq, 0.11 mmol) in THF/H₂O 1:1 (6 mL), LiOH (14 mg, 5 Eq, 0.57 mmol) was added. The reaction was stirred at rt for 1h and then it was diluted with EtOAc and HCl 0.5N causing precipitation of the desired product that was collected by vacuum filtration. The material was suspended in DCM (0.2 mL) and TFA (0.2 mL) was added, the reaction was stirred for 1 h and then the solvent was removed in vacuo, the residue was redissolved and evaporated three times to give the desired product as a TFA salt.

¹H NMR (400 MHz, DMSO-d₆) δ 13.32-13.18 (m, 1H), 9.96 (s, 1H), 9.20-9.08 (m, 2H), 8.84-8.78 (m, 1H), 8.74 (d, J=6.1 Hz, 1H), 8.34-8.28 (m, 1H), 8.01 (d, J=8.6 Hz, 1H), 7.76 (dd, J=8.4, 2.3 Hz, 1H), 7.63 (ddd, J=8.4, 7.0, 1.0 Hz, 1H), 7.50-7.39 (m, 2H), 5.68 (q, J=9.1 Hz, 2H), 2.98-2.88 (m, 4H), 1.88-1.75 (m, 4H), 1.62 (br d, J=4.1 Hz, 2H)

UPLC-MS: Rₜ=0.86 min. MS (ESI+): m/z=567.7 [M+H]⁺.

### Example 31: 2-(4-(piperidin-1-yl)-3-(1-(2,2,2-trifluoroethyl)-1H-indazole-3-carboxamido)benzamido)nicotinic acid

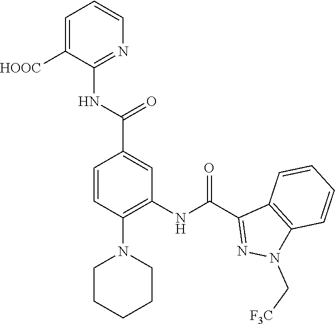 [SMILES: O=C(Nc1ncccc1C(=O)O)c1ccc(N2CCCCC2)c(NC(=O)c2nn(CC(F)(F)F)c3ccccc23)c1]

### Step 1: methyl 2-(4-(piperidin-1-yl)-3-(1-(2,2,2-trifluoroethyl)-1H-indazole-3-carboxamido)benzamido)nicotinate

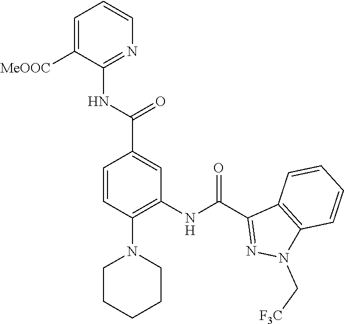 [SMILES: COC(=O)c1cccnc1NC(=O)c1ccc(N2CCCCC2)c(NC(=O)c2nn(CC(F)(F)F)c3ccccc23)c1]

The compound was synthesized following the coupling procedure described in coupling method described herein.

UPLC-MS (method 3): Rₜ=1.48 min. MS (ESI+): m/z=581.7 [M+H]⁺.

### Step 2: 2-(4-(piperidin-1-yl)-3-(1-(2,2,2-trifluormethyl)-1H-indazole-3-carboxanido)benzamido)nicotinic acid

To a solution of methyl 2-(4-(piperidin-1-yl)-3-(1-(2,2,2-trifluoroethyl)-1H-indazole-3-carboxamido)benzamido)nicotinate (113 mg, 1 Eq, 0.195 mmol) in THF/H₂O 1:1 (8 mL), LiOH (23 mg, 5 Eq, 0.97 mmol) was added. The reaction was stirred at rt for 1h, a precipitate formed the mixture was diluted with HCl 1N. The mixture was evaporated n vacuo, the residue thus obtained was diluted in DCM/MeOH/water and then filtered to give 90 mg of desired material (78%). Part of these (36.5 mg) were suspended in DCM (0.5 mL) and TFA (0.5 mL), the mixture was stirred for 1 hour at rt and then the solvent was removed in vacuo to obtain the desired product as a TFA salt.

¹H NMR (400 MHz, DMSO-d₆) δ 11.68-11.57 (m, 1H), 9.98 (s, 1H), 9.04 (d, J=2.0 Hz, 1H), 8.60 (dd, 1=4.8, 2.0 Hz, 1H), 8.32 (dt, J=8.1, 0.9 Hz, 1H), 8.27 (dd, =7.7, 1.9 Hz, 1H), 8.00 (d, J=8.6 Hz, 1H), 7.80 (dd, J=8.4, 2.3 Hz, 1H), 7.63 (ddd, J=8.4, 7.2, 1.1 Hz, 1H), 7.47-7.42 (m, 1H), 7.40 (d, J=8.4 Hz, 1H), 7.37-7.31 (m, 1H), 5.67 (q, J=9.0 Hz, 2H), 2.96-2.88 (m, 4H), 1.88-1.75 (m, 4H), 1.61 (br s, 2H).

UPLC-MS: Rₜ=0.82 min. MS (ESI+): m/z=567.7 [M+H]⁺.

### Example 32: 2-(2-(4-(Piperidin-1-yl)-3-(1-(2,2,2-trifluoroethyl)-1H-indazole-3-carboxamido)benzamido)phenyl)acetic acid

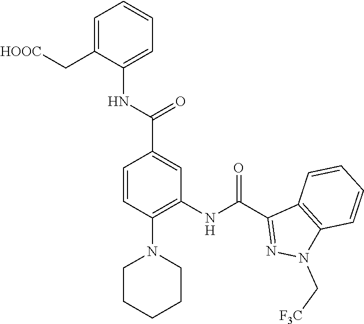 [SMILES: O=C(O)Cc1ccccc1NC(=O)c1ccc(N2CCCCC2)c(NC(=O)c2nn(CC(F)(F)F)c3ccccc23)c1]

Sodium 2-(2-aminophenyl)acetate (30 mg, 1.0 Eq, 0.17 mmol) was dissolved in THF (2 mL) and a saturated solution of NaHCO₃ (1 mL), while vigorously stirring the solution was cooled with an ice bath and 4-(piperidin-1-yl)-3-(1-(2,2,2-trifluoroethyl)-1H-indazole-3-carboxamido)benzoyl chloride (80 mg, 1 Eq, 0.17 mmol) was added, the reaction was stirred overnight. The reaction was diluted with EtOAc and water, the organic phase was evaporated, and the residue was purified by preparative RP-HPLC, gradient C to obtain the desired material (39 mg, 39%).

¹H NMR (400 MHz, DMSO-d₆) δ 10.00 (s, 1H), 9.02 (d, J=2.0 Hz, 1H), 8.37-8.29 (m, 1H), 7.99 (d, J=8.6 Hz, 1H), 7.86 (dd, J=8.4, 2.0 Hz, 1H), 7.73 (d, J=7.6 Hz, 1H), 7.62 (ddd, J=8.4, 7.0, 1.0 Hz, 1H), 7.44 (ddd, J=8.0, 7.2, 0.8 Hz, 1H), 7.33 (d, J=8.4 Hz, 1H), 7.28-7.20 (m, 2H), 7.14-7.05 (m, 1H), 5.67 (q, J=9.0 Hz, 2H), 3.51 (s, 2H), 2.94-2.87 (m, 4H), 1.86-1.76 (m, 4H), 1.60 (br s, 2H)

UPLC-MS: Rₜ=0.86 min. MS (EST+): m/z=580.7 [M+H]⁺.

The following examples were prepared in a methodology analogous to the Examples 28-32 with suitable reagents, precursors and starting materials unless otherwise stated.

| Compound# | Name | Structure/Coupling method | Analytical data |
|---|---|---|---|
| 3 | 5-(2-(4- (piperidin-1-yl)- 3-(1-(2,2,2- trifluoroethyl)- 1H-indazole-3- carboxamido) benzamido)-2- fluorophenyl) acetic acid | 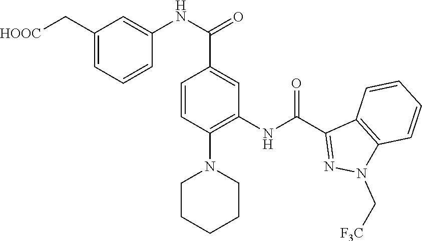 [SMILES: O=C(O)Cc1cccc(NC(=O)c2ccc(N3CCCCC3)c(NC(=O)c3nn(CC(F)(F)F)c4ccccc34)c2)c1] | ¹H NMR (400 MHz, DMSO-d₆) δ 10.15 (s, 1H), 10.02 (s, 1H), 8.97 (d, J = 2.0 Hz, 1H), 8.38-8.29 (m, 1H), 8.00 (d, J = 8.6 Hz, 1H), 7.73 (dd, J = 8.4, 2.0 Hz, 1H), 7.66- 7.58 (m, 2H), 7.55 (d, J = 1.5 Hz, 1H), 7.48-7.41 (m, 1H), 7.37 (d, J = 8.4 Hz, 1H), 7.16 (t, J = 7.9 Hz, 1H), 6.93 (d, J = 7.9 Hz, 1H), 5.67 (q, J = 9.1 Hz, 2H), 3.16 (s, 2H), 2.94-2.87 (m, 4H), 1.86-1.75 (m, 4H), 1.61 (br s, 2H) UPLC-MS: Rₜ = 0.89 min. MS (ESI+): m/z = 580.7 [M + H]⁺. |
| | | Example 28 | |
| 6 | 2-(5-fluoro-2-(4- (piperidin-1-yl)- 3-(1-(2,2,2- trifluoroethyl)- 1H-indazole-3- carboxamido) benzamido) phenyl) propanoic acid | 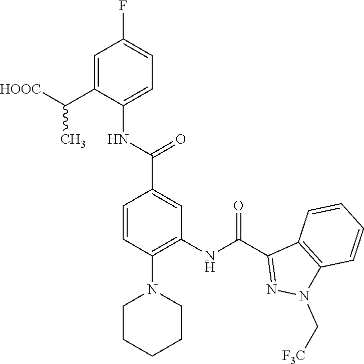 [SMILES: CC(C(=O)O)c1cc(F)ccc1NC(=O)c1ccc(N2CCCCC2)c(NC(=O)c2nn(CC(F)(F)F)c3ccccc23)c1] | ¹H NMR (400 MHz, DMSO-d₆) δ 10.01 (d, J = 5.1 Hz, 2H), 9.00 (d, J = 2.0 Hz, 1H), 8.32 (d, J = 8.1 Hz, 1H), 8.00 (d, J = 8.6 Hz, 1H), 7.77 (dd, J = 8.4, 2.0 Hz, 1H), 7.62 (ddd, J = 8.4, 7.1, 1.0 Hz, 1H), 7.44 (t, J = 7.5 Hz, 1H), 7.40-7.31 (m, 2H), 7.21-7.12 (m, 2H), 5.67 (q, J = 8.9 Hz, 2H), 3.98-3.89 (m, 1H), 2.95-2.86 (m, 4H), 1.82 (dt, J = 9.9, 5.3 Hz, 4H), 1.61 (br s, 2H), 1.40 (d, J = 7.1 Hz, 3H) UPLC-MS: Rₜ = 0.92 min. MS (ESI+): m/z = 612.8 [M + H]⁺. |
| | | Example 29 | |
| 10 | 3-(4-(piperidin- 1-yl)-3-(1-(2,2,2- trifluoroethyl)- 1H-indazole-3- carboxamido) benzamido) benzoic acid | 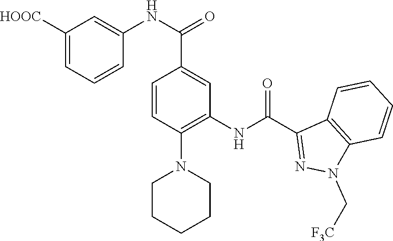 [SMILES: O=C(O)c1cccc(NC(=O)c2ccc(N3CCCCC3)c(NC(=O)c3nn(CC(F)(F)F)c4ccccc34)c2)c1] | ¹H NMR (400 MHz, DMSO-d₆) δ 10.37 (s, 1H), 10.01 (s, 1H), 8.99 (d, J = 2.0 Hz, 1H), 8.37-8.28 (m, 2H), 8.04-7.95 (m, 2H), 7.76 (dd, J = 8.2, 2.2 Hz, 1H), 7.68-7.56 (m, 2H), 7.47-7.34 (m, 3H), 5.74- 5.60 (m, 2H), 2.94-2.87 (m, 4H), 1.85-1.76 (m, 4H), 1.61 (br s, 2H) LC-MS: Rₜ = 1.48 min. MS (ESI+): m/z = 566.4 [M + H]⁺. |
| | | Example 28 | |
| 11 | 4-(4-(piperidin- 1-yl)-3-(1-(2,2,2- trifluoroethyl)- 1H-indazole-3- carboxamido) benzamido) benzoic acid | 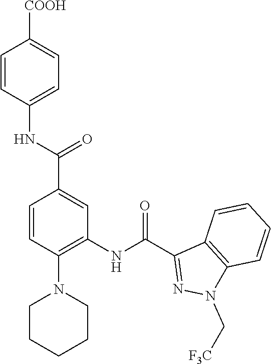 [SMILES: O=C(O)c1ccc(NC(=O)c2ccc(N3CCCCC3)c(NC(=O)c3nn(CC(F)(F)F)c4ccccc34)c2)cc1] | ¹H NMR (400 MHz, DMSO-d₆) δ 10.41 (s, 1H), 10.02 (s, 1H), 8.98 (d, J = 2.0 Hz, 1H), 8.38-8.30 (m, 1H), 8.00 (d, J = 8.6 Hz, 1H), 7.92- 7.84 (m, 2H), 7.80 (d, J = 8.6 Hz, 2H), 7.74 (dd, J = 8.4, 2.0 Hz, 1H), 7.63 (ddd, J = 8.4, 7.0, 1.0 Hz, 1H), 7.47-7.42 (m, 1H), 7.39 (d, J = 8.4 Hz, 1H), 5.67 (q, J = 9.0 Hz, 2H), 2.94-2.87 (m, 4H), 1.87- 1.76 (m, 4H), 1.61 (br s, 2H) UPLC-MS: Rₜ = 0.93 min. MS (ESI+): m/z = 566.7 [M + H]⁺. |
| | | Example 29 | |
| 12 | 2-(3-fluoro-4-(4- (piperidin-1-yl)- 3-(1-(2,2,2- trifluoroethyl)- 1H-indazole-3- carboxamido) benzamido) phenyl) acetic acid | 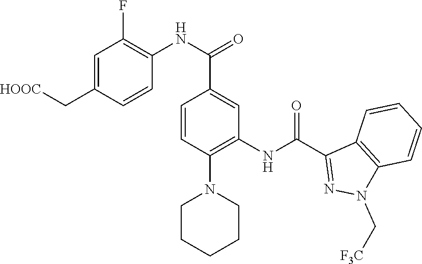 [SMILES: O=C(O)Cc1ccc(NC(=O)c2ccc(N3CCCCC3)c(NC(=O)c3nn(CC(F)(F)F)c4ccccc34)c2)c(F)c1] | ¹H NMR (400 MHz, DMSO-d₆) δ 10.06 (s, 1H), 10.00 (s, 1H), 8.99 (d, J = 2.0 Hz, 1H), 8.33 (d, J = 8.1 Hz, 1H), 8.00 (d, J = 8.6 Hz, 1H), 7.75 (dd, ) = 8.4, 2.0 Hz, 1H), 7.62 (ddd, J = 8.4, 7.1, 1.0 Hz, 1H), 7.52-7.41 (m, 2H), 7.37 (d, J = 8.4 Hz, 1H), 7.19 (dd, J = 11.7, 1.8 Hz, 1H), 7.09 (dd, J = 8.4, 1.5 Hz, 1H), 5.67 (q, J = 9.0 Hz, 2H), 3.55 (s, 2H), 2.93-2.86 (m, 4H), 1.86-1.75 (m, 4H), 1.61 (br s, 2H) LC-MS: Rₜ = 1.47 min. MS (ESI+): m/z = 598.4 [M + H]⁺. |
| | | Example 32, followed by | |
| | | TFA deprotection of tert- | |
| | | butyl ester | |
| 16 | N-(5-((2-(2- amino-2- oxoethyl) phenyl) carbamoyl)-2- (piperidin-1- yl)phenyl)-1- (2,2,2- trifluoroethyl)- 1H-indazole-3- carboxamide | 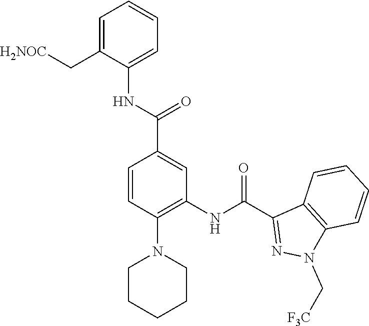 [SMILES: NC(=O)Cc1ccccc1NC(=O)c1ccc(N2CCCCC2)c(NC(=O)c2nn(CC(F)(F)F)c3ccccc23)c1] | ¹H NMR (400 MHz, DMSO-d₆) δ 11.18 (s, 1H), 10.00 (s, 1H), 9.05 (d, J = 2.0 Hz, 1H), 8.33 (dt, J = 8.2, 1.0 Hz, 1H), 8.00 (d, J = 8.6 Hz, 1H), 7.94 (br d, J = 1.8 Hz, 1H), 7.88 (d, J = 8.1 Hz, 1H), 7.80 (dd, J = 8.4, 2.3 Hz, 1H), 7.63 (ddd, J = 8.4, 7.0, 1.0 Hz, 1H), 7.44 (td, J = 7.6, 0.8 Hz, 1H), 7.38 (d, J = 8.4 Hz, 1H), 7.34-7.28 (m, 3H), 7.17- 7.11 (m, 1H), 5.67 (q, J = 9.0 Hz, 2H), 3.55 (s, 2H), 2.94-2.87 (m, 4H), 1.86-1.76 (m, 4H), 1.61 (br d, J = 3.8 Hz, 2H) UPLC-MS: Rₜ = 1.33 min. MS (ESI+): m/z = 579.7 [M + H]⁺. |
| | | Example 28 | |
| 18 | 2-((4-(piperidin- 1-yl)-3-(1-(2,2,2- trifluoroethyl)- 1H-indazole-3- carboxamido) benzamido) methyl) benzoic acid | 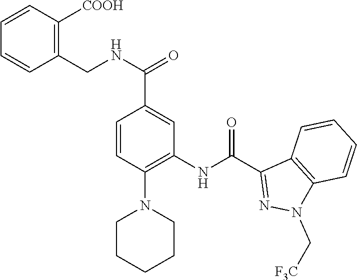 [SMILES: O=C(NCc1ccccc1C(=O)O)c1ccc(N2CCCCC2)c(NC(=O)c2nn(CC(F)(F)F)c3ccccc23)c1] | ¹H NMR (400 MHz, DMSO-d₆) δ 10.00 (s, 1H), 9.00 (br t, J = 4.8 Hz, 1H), 8.94 (d, J = 2.0 Hz, 1H), 8.32 (d, J = 8.4 Hz, 1H), 7.99 (d, J = 8.6 Hz, 1H), 7.88 (dd, J = 7.7, 1.1 Hz, 1H), 7.68 (dd, J = 8.4, 2.0 Hz, 1H), 7.62 (ddd, J = 8.4, 7.0, 1.0 Hz, 1H), 7.56-7.49 (m, 1H), 7.46-7.30 (m, 4H), 5.66 (q, J = 9.0 Hz, 2H), 4.81 (br d, J = 5.6 Hz, 2H), 2.92-2.83 (m, 4H), 1.85-1.74 (m, 4H), 1.60 (br s, 2H). LC-MS: Rₜ = 1.47 min. MS (ESI+): m/z = 580.4 [M + H]⁺. |
| | | Example 28 | |
| 19 | 3-((4-(piperidin- 1-yl)-3-(1-(2,2,2- trifluoroethyl)- 1H-indazole-3- carboxamido) benzamido) methyl) benzoic acid | 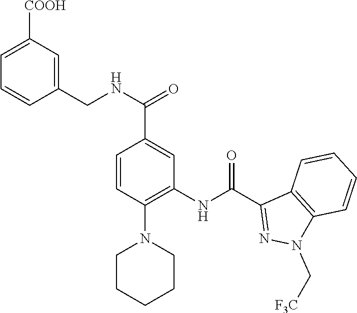 [SMILES: O=C(O)c1cccc(CNC(=O)c2ccc(N3CCCCC3)c(NC(=O)c3nn(CC(F)(F)F)c4ccccc34)c2)c1] | ¹H NMR (400 MHz, DMSO-d₆) δ 9.99 (s, 1H), 9.10 (t, J = 5.8 Hz, 1H), 8.93 (d, J = 2.3 Hz, 1H), 8.35- 8.28 (m, 1H), 7.99 (d, J = 8.6 Hz, 1H), 7.92 (s, 1H), 7.86-7.78 (m, 1H), 7.70-7.53 (m, 3H), 7.48- 7.39 (m, 2H), 7.34 (d, J = 8.4 Hz, 1H), 5.67 (q, J = 9.0 Hz, 2H), 4.54 (d, J = 5.8 Hz, 2H), 2.91-2.84 (m, 4H), 1.84-1.75 (m, 4H), 1.65 1.54 (m, 2H) LC-MS: Rₜ = 1.43 min. MS (ESI+): m/z = 580.4 [M + H]⁺. |
| | | Example 28 | |
| 22 | 2-(2-(4- (piperidin-1-yl)- 3-(1-(2,2,2- trifluoroethyl)- 1H-indazole-3- carboxamido) benzamido)-5- (trifluoromethyl) phenyl)acetic acid | 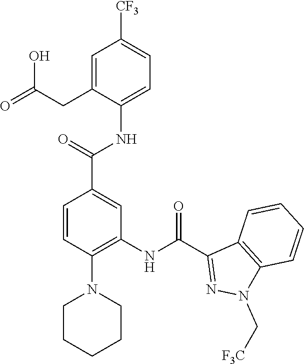 [SMILES: O=C(O)Cc1cc(C(F)(F)F)ccc1NC(=O)c1ccc(N2CCCCC2)c(NC(=O)c2nn(CC(F)(F)F)c3ccccc23)c1] | ¹H NMR (400 MHz, DMSO-d₆) δ 10.19 (s, 1H), 10.01 (s, 1H), 9.00 (d, J = 2.0 Hz, 1H), 8.32 (d, J = 8.4 Hz, 1H), 8.00 (d, J = 8.6 Hz, 1H), 7.78-7.72 (m, 3H), 7.71-7.59 (m, 2H), 7.48-7.37 (m, 2H), 5.67 (q, J = 9.0 Hz, 2H), 3.83 (s, 2H), 2.96-2.85 (m, 4H), 1.88-1.77 (m, 4H), 1.62 (br d, J = 2.8 Hz, 2H) UPLC-MS (method 3): Rₜ = 0.97 min. MS (ESI+): m/z = 648.7 [M + H]⁺. |
| | | Example 29 | |
| 23 | 2-(5-chloro-2-(4- (piperidin-1-yl)- 3-(1-(2,2,2- trifluoroethyl)- 1H-indazole-3- carboxamido) benzamido) phenyl) acetic acid | 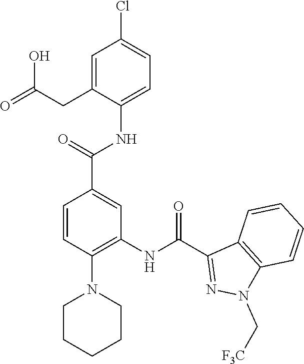 [SMILES: O=C(O)Cc1cc(Cl)ccc1NC(=O)c1ccc(N2CCCCC2)c(NC(=O)c2nn(CC(F)(F)F)c3ccccc23)c1] | 1H NMR (400 MHz, DMSO-d₆) δ 10.02 (d, J = 6.1 Hz, 2H), 8.98 (d, J = 2.0 Hz, 1H), 8.32 (d, J = 8.1 Hz, 1H), 8.00 (d, J = 8.6 Hz, 1H), 7.72 (dd, J = 8.4, 2.0 Hz, 1H), 7.62 (ddd, J = 8.4, 7.0, 1.0 Hz, 1H), 7.50- 7.41 (m, 3H), 7.40-7.32 (m, 2H), 5.67 (q, J = 9.0 Hz, 2H), 3.69 (s, 2H), 2.95-2.86 (m, 4H), 1.85- 1.78 (m, 4H), 1.61 (br d, J = 2.5 Hz, 2H) UPLC-MS (method 3): Rₜ = 0.93 min. MS (ESI+): m/z = 614.6 [M + H]⁺. |
| | | Example 29 | |
| 25 | 2-(3-chloro-2-(4- (piperidin-1-yl)- 3-(1-(2,2,2- trifluoroethyl)- 1H-indazole-3- carboxamido) benzamido) phenyl) acetic acid | 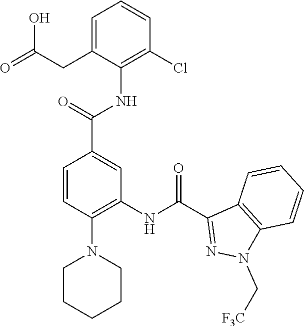 [SMILES: O=C(O)Cc1cccc(Cl)c1NC(=O)c1ccc(N2CCCCC2)c(NC(=O)c2nn(CC(F)(F)F)c3ccccc23)c1] | ¹H NMR (400 MHz, DMSO-d₆) δ 10.02 (s, 1H), 8.99 (d, J = 2.0 Hz, 1H), 8.32 (d, J = 8.1 Hz, 1H), 8.00 (d, J = 8.6 Hz, 1H), 7.81 (dd, J = 8.4, 2.0 Hz, 1H), 7.62 (td, J = 7.8, 0.9 Hz, 1H), 7.50-7.41 (m, 2H), 7.37 (d, J = 8.1 Hz, 1H), 7.34-7.18 (m, 2H), 5.67 (q, J = 9.0 Hz, 2H), 3.53 (s, 2H), 2.95-2.86 (m, 4H), 1.84-1.75 (m, 4H), 1.61 (br s, 2H) UPLC-MS (method 3): Rₜ = 0.88 min. MS (ESI+): m/z = 614.6 [M + H]⁺. |
| | | Example 29 | |
| 26 | 3-chloro-2-(4- (piperidin-1-yl)- 3-(1-(2,2,2- trifluoroethyl)- 1H-indazole-3- carboxamido) benzamido) benzoic acid | 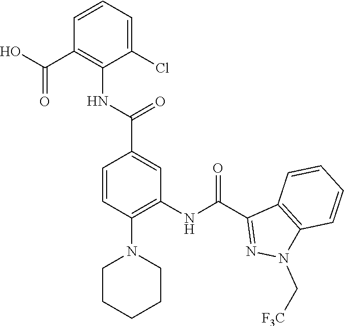 [SMILES: O=C(Nc1c(Cl)cccc1C(=O)O)c1ccc(N2CCCCC2)c(NC(=O)c2nn(CC(F)(F)F)c3ccccc23)c1] | ¹H NMR (400 MHz, DMSO-d₆) δ 11.66-10.98 (m, 1H), 11.86- 10.83 (m, 1H), 10.02 (s, 1H), 9.00 (d, J = 2.0 Hz, 1H), 8.32 (d, J = 8.1 Hz, 1H), 8.00 (d, J = 8.4 Hz, 1H), 7.86-7.73 (m, 2H), 7.70- 7.59 (m, 2H), 7.47-7.36 (m, 2H), 7.36-7.30 (m, 1H), 5.67 (q, J = 9.0 Hz, 2H), 3.34 (br s, 7H), 2.97- 2.84 (m, 4H), 1.89-1.74 (m, 4H), 1.72-1.50 (m, 3H) UPLC-MS (method 5): Rₜ = 0.95 min. MS (ESI+): m/z = 600.7 [M + H]⁺. |
| | | Example 32 | |
| 27 | 3-fluoro-2-(4- (piperidin-1-yl)- 3-(1-(2,2,2- trifluoroethyl)- 1H-indazole-3- carboxamido) benzamido) benzoic acid | 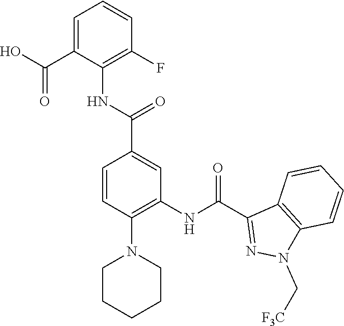 [SMILES: O=C(Nc1c(F)cccc1C(=O)O)c1ccc(N2CCCCC2)c(NC(=O)c2nn(CC(F)(F)F)c3ccccc23)c1] | ¹H NMR (400 MHz, DMSO-d₆) δ 11.90-11.17 (m, 1H), 12.10- 10.96 (m, 1H), 10.01 (s, 1H), 9.01 (d, J = 2.0 Hz, 1H), 8.32 (d, J = 8.1 Hz, 1H), 8.00 (d, J = 8.6 Hz, 1H), 7.76 (dd, J = 8.2, 2.2 Hz, 1H), 7.72-7.67 (m, 1H), 7.67-7.57 (m, 1H), 7.49-7.34 (m, 3H), 7.34- 7.24 (m, 1H), 5.67 (q, J = 9.0 Hz, 2H), 2.97-2.85 (m, 4H), 1.89- 1.73 (m, 4H), 1.72-1.50 (m, 2H) UPLC-MS: Rₜ = 0.90 min. MS (ESI+): m/z = 584.6 [M + H]⁺. |
| | | Example 32 | |
| 28 | 2-(5-fluoro-2-(4- (piperidin-1-yl)- 3-(1-(2,2,2- trifluoroethyl)- 1H-indazole-3- carboxamido) benzamido) phenyl) butanoic acid | 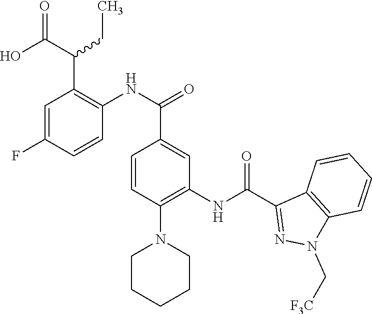 [SMILES: CCC(C(=O)O)c1cc(F)ccc1NC(=O)c1ccc(N2CCCCC2)c(NC(=O)c2nn(CC(F)(F)F)c3ccccc23)c1] | ¹H NMR (400 MHz, DMSO-d₆) δ 10.02 (d, J = 2.8 Hz, 2H), 9.00 (d, J = 2.0 Hz, 1H), 8.32 (dt, J = 8.2, 1.0 Hz, 1H), 8.00 (d, J = 8.6 Hz, 1H), 7.76 (dd, J = 8.2, 2.2 Hz, 1H), 7.62 (ddd, J = 8.5, 7.1, 1.1 Hz, 1H), 7.48-7.35 (m, 3H), 7.24-7.08 (m, 2H), 5.67 (q, J = 9.0 Hz, 2H), 3.78- 3.69 (m, 1H), 2.94-2.86 (m, 4H), 2.03-1.89 (m, 1H), 1.86- 1.77 (m, 4H), 1.74 (dt, J = 13.9, 6.7 Hz, 1H), 1.61 (br s, 2H), 0.86 (t, J = 7.4 Hz, 3H) UPLC-MS (method 3): Rₜ = 0.98 min. MS (ESI+): m/z = 626.7 [M + H]⁺. |
| | | Example 29 | |
| 30 | 2-(5-fluoro-2-(4- (piperidin-1-yl)- 3-(1-(2,2,2- trifluoroethyl)- 1H-indazole-3- carboxamido) benzamido) phenyl)-2- methylpropanoic acid | 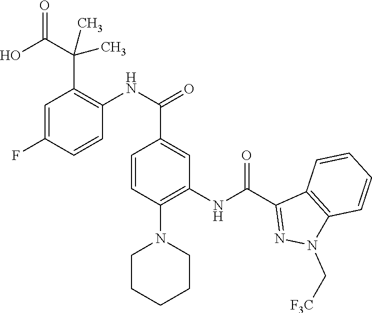 [SMILES: CC(C)(C(=O)O)c1cc(F)ccc1NC(=O)c1ccc(N2CCCCC2)c(NC(=O)c2nn(CC(F)(F)F)c3ccccc23)c1] | ¹H NMR (400 MHz, DMSO-d₆) δ 12.29 (br s, 1H), 10.01 (s, 1H), 9.19 (s, 1H), 8.95 (d, J = 2.0 Hz, 1H), 8.32 (d, J = 8.1 Hz, 1H), 8.00 (d, J = 8.6 Hz, 1H), 7.70 (dd, J = 8.4, 1.8 Hz, 1H), 7.66-7.58 (m, 1H), 7.44 (t, J = 7.5 Hz, 1H), 7.37-7.24 (m, 3H), 7.17 (td, J = 8.3, 2.9 Hz, 1H), 5.67 (q, J = 9.1 Hz, 2H), 2.95- 2.86 (m, 4H), 1.85-1.76 (m, 4H), 1.61 (br s, 2H), 1.48 (s, 6H). UPLC-MS: Rₜ = 0.87 min. MS (ESI+): m/z = 626.7 [M + H]⁺. |
| | | Example 29 | |
| 31 | 1-(5-fluoro-2-(4- (piperidin-1-yl)- 3-(1-(2,2,2- trifluoroethyl)- 1H-indazole-3- carboxamido) benzamido) phenyl) cyclopropane-1- carboxylic acid | 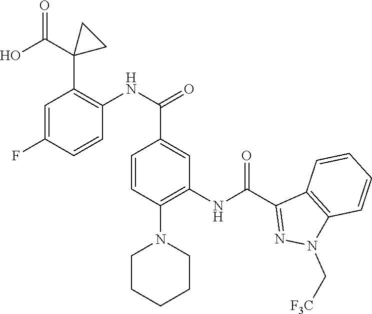 [SMILES: O=C(Nc1ccc(F)cc1C1(C(=O)O)CC1)c1ccc(N2CCCCC2)c(NC(=O)c2nn(CC(F)(F)F)c3ccccc23)c1] | ¹H NMR (400 MHz, DMSO-d₆) δ 10.01 (s, 1H), 9.00 (d, J = 2.0 Hz, 1H), 8.33 (d, J = 8.1 Hz, 1H), 8.00 (d, J = 8.6 Hz, 1H), 7.73-7.58 (m, 3H), 7.45 (t, J = 7.6 Hz, 1H), 7.36 (d, J = 8.1 Hz, 1H), 7.22-7.11 (m, 2H), 5.67 (q, J = 8.9 Hz, 2H), 2.94- 2.85 (m, 4H), 1.88-1.76 (m, 4H), 1.61 (br s, 2H), 1.45 (br d, J = 2.0 Hz, 2H) UPLC-MS (method 5): Rₜ = 0.92 min. MS (ESI+): m/z = 624.7 [M + H]⁺ |
| | | Example 29 | |
| 33 | 2-(5-methyl-2- (4-(piperidin-1- yl)-3-(1-(2,2,2- trifluoroethyl)- 1H-indazole-3- carboxamido) benzamido) phenyl) acetic acid | 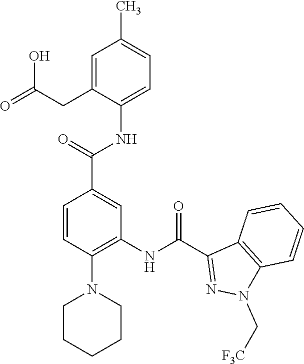 [SMILES: Cc1ccc(NC(=O)c2ccc(N3CCCCC3)c(NC(=O)c3nn(CC(F)(F)F)c4ccccc34)c2)c(CC(=O)O)c1] | ¹H NMR (400 MHz, DMSO-d₆) δ 10.04-9.99 (m, 1H), 9.90 (s, 1H), 8.97 (d, J = 2.0 Hz, 1H), 8.35-8.30 (m, 1H), 8.00 (d, J = 8.6 Hz, 1H), 7.72 (dd, J = 8.4, 2.0 Hz, 1H), 7.62 (ddd, J = 8.4, 7.0, 1.0 Hz, 1H), 7.48- 7.41 (m, 1H), 7.39-7.29 (m, 2H), 7.16-7.09 (m, 2H), 5.67 (q, J = 9.0 Hz, 2H), 3.61 (s, 2H), 2.95- 2.86 (m, 4H), 2.31 (s, 3H), 1.86- 1.76 (m, 4H), 1.61 (br d, J = 3.3 Hz, 2H) UPLC-MS: Rₜ = 0.87 min. MS (ESI+): m/z = 594.5 [M + H]⁺. |
| | | Example 29 | |
| 37 | 2-(2-fluoro-6-(4- (piperidin-1-yl)- 3-(1-(2,2,2- trifluoroethyl)- 1H-indazole-3- carboxamido) benzamido) phenyl) acetic acid | 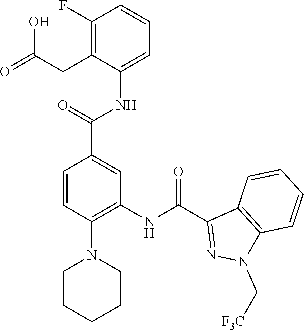 [SMILES: O=C(O)Cc1c(F)cccc1NC(=O)c1ccc(N2CCCCC2)c(NC(=O)c2nn(CC(F)(F)F)c3ccccc23)c1] | ¹H NMR (400 MHz, DMSO-d₆) δ 10.13 (s, 1H), 10.01 (s, 1H), 8.98 (d, J = 2.0 Hz, 1H), 8.35-8.29 (m, 1H), 8.00 (d, J = 8.6 Hz, 1H), 7.73 (dd, J = 8.4, 2.0 Hz, 1H), 7.62 (ddd, J = 8.4, 7.2, 1.1 Hz, 1H), 7.48- 7.41 (m, 1H), 7.40-7.32 (m, 2H), 7.31-7.26 (m, 1H), 7.17-7.06 (m, 1H), 5.67 (q, J = 9.0 Hz, 2H), 3.65 (s, 2H), 2.94-2.86 (m, 4H), 1.86- 1.76 (m, 4H), 1.66-1.57 (m, 2H) UPLC-MS: Rₜ = 0.88 min. MS (ESI+): m/z = 598.5 [M + H]⁺. |
| | | Example 29 | |
| 38 | 2-(4-fluoro-2-(4- (piperidin-1-yl)- 3-(1-(2,2,2- trifluoroethyl)- 1H-indazole-3- carboxamido) benzamido) phenyl) acetic acid | 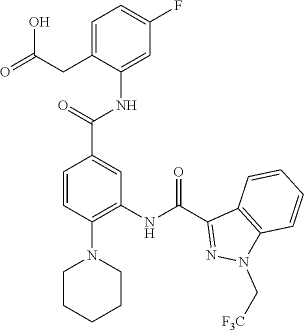 [SMILES: O=C(O)Cc1ccc(F)cc1NC(=O)c1ccc(N2CCCCC2)c(NC(=O)c2nn(CC(F)(F)F)c3ccccc23)c1] | ¹H NMR (400 MHz, DMSO-d₆) δ 10.00 (s, 1H), 9.01 (d, J = 2.0 Hz, 1H), 8.33 (d, J = 8.1 Hz, 1H), 8.00 (d, J = 8.4 Hz, 1H), 7.82 (dd, J = 8.2, 2.2 Hz, 1H), 7.66-7.55 (m, 2H), 7.44 (t, J = 7.4 Hz, 1H), 7.35 (d, J = 8.4 Hz, 1H), 7.29 (dd, J = 8.4, 6.6 Hz, 1H), 6.97 (td, J = 8.4, 2.8 Hz, 1H), 5.67 (q, J = 9.0 Hz, 2H), 3.57 (s, 2H), 2.93-2.86 (m, 4H), 1.85-1.76 (m, 4H), 1.61 (br s, 2H) UPLC-MS (method 5): Rₜ = 0.93 min. MS (ESI+): m/z = 598.6 [M + H]⁺. |
| | | Example 29 | |

### Example 33: 2,2-difluoro-2-(2-(4-(piperidin-1-yl)-3-(1-(2,2,2-trifluoroethyl)-1H-indazole-3-carboxamido)benzamido)phenyl)acetic acid

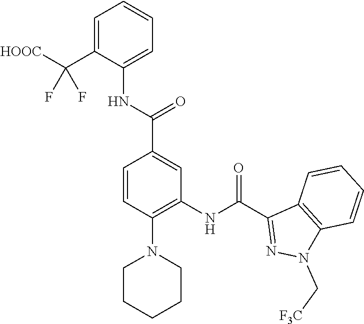 [SMILES: O=C(Nc1ccccc1C(F)(F)C(=O)O)c1ccc(N2CCCCC2)c(NC(=O)c2nn(CC(F)(F)F)c3ccccc23)c1]

### Step 1: 3,3-Difluoroindolin-2-one

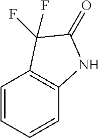 [SMILES: O=C1Nc2ccccc2C1(F)F]

A mixture of ethyl 2,2-difluoro-2-(2-nitrophenyl)acetate (150 mg, 1 Eq, 612 μmol), Pd—C (130 mg, 10% Wt, 0.2 Eq, 122 μmol) in EtOAc (5 mL) was stirred under an H₂ (2.47 mg, 2 Eq, 1.22 mmol) atmosphere at 25° C. for 17 hours. The mixture was filtered over a pad of celite, the organic solvent was evaporated to dryness to obtain 103 mg of residue (612 μmol, q.) that was directly used in the next step without further purification.

¹H NMR (400 MHz, DMSO-d₆) δ 11.20 (br s, 1H), 7.65 (dd, J=7.5, 1.9 Hz, 1H), 7.55-7.49 (m, 1H), 7.16 (t, J=7.6 Hz, 1H), 6.99 (dt, J=7.9, 0.8 Hz, 1H).

UPLC-MS: Rₜ=0.73 min. MS (ESI+): m/z=168.2 [M−H]—.

### Step 2: 2,2-difluoro-2-(2-(4-(piperidin-1-y)-3-(1-(2,2,2-trifluoroethyl)-1H-indazole-3-carboxarido)benzamido)phenyl)acetic acid

4-(Piperidin-1-yl)-3-(1-(2,2,2-trifluoroethyl)-1H-indazole-3-carboxamido)benzoyl chloride (80 mg, 1 Eq, 0.17 mmol) was dissolved in DCM (1.4 mL), triethylamine (17 mg, 1 Eq, 0.17 mmol) was added, followed by 3,3-difluoroindolin-2-one (29 mg, 1.0 Eq, 0.17 mmol). The reaction was stirred at room temperature for 72 hours, then it was evaporated in vacuo, the residue was purified by preparative RP-HPLC, gradient C to obtain the desired material (35 mg, 33%).

¹H NMR (400 MHz, DMSO-d&) S 9.02 (d, J=2.0 Hz, 1H), 8.33 (d, J=8.1 Hz, 1H), 8.02 (dd, J=17.0, 8.4 Hz, 2H), 7.89 (dd, J=8.4, 2.0 Hz, 1H), 7.62 (td, J=7.7, 1.0 Hz, 1H), 7.57-7.49 (m, 2H), 7.44 (t, J=7.6 Hz, 1H), 7.38 (d, J=8.4 Hz, 1H), 7.27 (t, J=7.7 Hz, 1H), 5.67 (q, J=9.3 Hz, 2H), 3.00-2.86 (m, 4H), 1.90-1.76 (m, 4H), 1.61 (br s, 2H).

UPLC-MS (method 5): Rₜ=0.95 min. MS (ESI+): m/z=616.8 [M+H]⁺.

### Example 34: 3-(2-(4-(piperidin-1-yl)-3-(1-(2,2,2-trifuoroethyl)-1H-indazole-3-carboxamido)benzamido)phenyl)propanoic acid

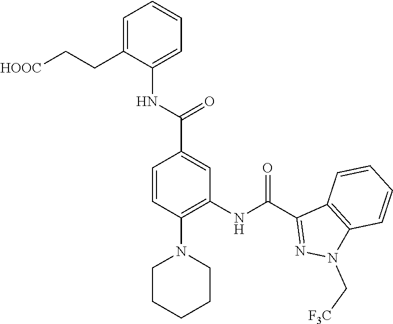 [SMILES: O=C(O)CCc1ccccc1NC(=O)c1ccc(N2CCCCC2)c(NC(=O)c2nn(CC(F)(F)F)c3ccccc23)c1]

### Step 1: Tert-butyl 3-(2-(4-(piperidin-1-yl)-3-(1-(2,2,2-trifluoroethyl)-1H-indazole-3-carboxanido)benzamido)phenyl)propanoate

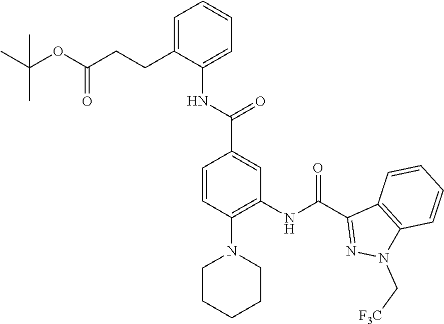 [SMILES: CC(C)(C)OC(=O)CCc1ccccc1NC(=O)c1ccc(N2CCCCC2)c(NC(=O)c2nn(CC(F)(F)F)c3ccccc23)c1]

Synthesized using coupling method described herein.

UPLC-MS: Rₜ=1.63 min. MS (ESI+): m/z=650.8 [M+H].

### Step 2: 3-(2-(4-(piperidin-1-yl)-3-(1-(2,2,2-trifluoroethyl)-1H-indazole-3-carboxanido)benzamido)phenyl)propanoic acid

Obtained from the corresponding tert-butyl ester using the procedure described in coupling method described herein.

¹H NMR (400 MHz, DMSO-d₆) δ 10.13 (br s, 1H), 10.02 (s, 1H), 9.01 (d, J=2.0 Hz, 1H), 8.33 (dt, J=8.2, 1.0 Hz, 1H), 8.00 (d, J=8.6 Hz, 1H), 7.79 (dd, J=8.4, 2.0 Hz, 1H), 7.62 (ddd, J=8.4, 7.2, 1.1 Hz, 1H), 7.44 (td, J=7.6, 0.8 Hz, 1H), 7.40-7.17 (m, 5H), 5.67 (q, J=9.0 Hz, 2H), 2.94-2.87 (m, 4H), 2.84 (t, J=7.6 Hz, 2H), 2.58-2.52 (m, 2H), 1.87-1.77 (m, 4H), 1.61 (br s, 2H).

UPLC-MS: Rₜ=0.93 min. MS (ESI+): m/z=594.8 [M+H]˜.

### Example 35: LPL ANGPTL4 Protection Assay

LPL is inactivated by angiopoietin-like 4 (ANGPTL4). Compounds of the present disclosure were for activity using the following protection assay. Briefly, the assay was performed in a black opaque 96-well microtiter plate with final volume of 100 μL using a buffer consisting of 50 mM HEPES (pH 7.2), 150 mM NaCl and 0.03% Triton X-100. Compounds were prepared as 10 mM stocks in DMSO, with subsequent serial dilutions performed in DMSO. Two microliters of test compounds were added to the microtiter plate, followed by 49-PL of LPL (native bovine milk protein affinity purified using heparin-affinity chromatography) in assay buffer. The plate contents were mixed using an orbital shaker and allowed to incubate for 5 minutes at room temperature. The enzymatic assay was initiated by the addition of a mixture of 49 μL of 50 μM resorufin butyrate (Santa Cruz #sc-208302; prepared from a 10 mM DMSO stock) and ANGPTL4 (N-terminal GST construct consisting of residues 26-164 expressed in E. coli) in assay buffer. The final concentrations of LPL and ANGPTL4 were 2.5 nM and 20 nM, respectively. The assay was allowed to proceed at 30° C. for 25 minutes with kinetic monitoring (Ex560/Em585) followed by an endpoint reading which was used for analysis.

Data was analyzed in Graphpad Prism (version 6.0, GraphPad Software, Inc., San Diego, CA) using a variable slope four parameter fit of the equation:

Y=Bottom+(Top-Bottom)/(1+10\hat{ }((Log EC50−X)*HillSlope)),

where X is the concentration of test compound, Y corresponds to the amount of product expressed in relative fluorescence units, and Top and Bottom refer to the plateaus of the curve. Percent activation (protection) of test compound was calculated relative to zero effect wells (ZEW; LPL, ANGPTL4 and substrate) and maximum effect wells (MEW; LPL plus substrate): ((test well−ZEW)/(MEW−ZEW))*100.

TABLE 1
Represents LPL activation activity of compounds of the present disclosure.
LPL Fluorescence Assay

| | | Compound |
|---|---|---|
| Compound | | Name EC₅₀ |
| No. | Compound Name | (uM) |
| 158 | 2-(5-fluoro-2-(4-(piperidin-1-yl)-3-(1-(2,2,2-trifluoroethyl)-1H- | 1.01 |
| | indazole-3-carboxamido)benzamido)phenyl)acetic acid; | |
| 159 | 2-(2-(3-(1-benzyl-1H-indazole-3-carboxamido)-4-(piperidin-1- | 0.85 |
| | yl)benzamido)-5-fluorophenyl)acetic acid; | |
| 160 | 2-(2-(3-(1-ethyl-1H-indazole-3-carboxamido)-4-(piperidin-1- | 1.66 |
| | yl)benzamido)-5-fluorophenyl)acetic acid; | |
| 161 | 2-(2-(4-(piperidin-1-yl)-3-(4,5,6,7-tetrahydropyrazolo[1,5-a]pyridine- | 3.20 |
| | 2-carboxamido)benzamido)phenyl)acetic acid; | |
| 162 | 2-(5-fluoro-2-(3-(1-isopropyl-1H-indazole-3-carboxamido)-4- | 3.25 |
| | (piperidin-1-yl)benzamido)phenyl)acetic acid; | |
| 163 | 2-(2-(3-(1-methyl-5-phenyl-1H-pyrazole-3-carboxamido)-4- | 4.13 |
| | (piperidin-1-yl)benzamido)phenyl)acetic acid | |
| 164 | N-(5-((4-(1H-tetrazol-5-yl)phenyl)carbamoyl)-2-(piperidin-1- | 5.16 |
| | yl)phenyl)-1-(2,2,2-trifluoroethyl)-1H-indazole-3-carboxamide; | |
| 165 | N-(5-((4-(1H-tetrazol-5-yl)phenyl)carbamoyl)-2-(piperidin-1- | 7.99 |
| | yl)phenyl)-6-fluoro-1-methyl-1H-indazole-3-carboxamide; | |
| 166 | N-(5-((2-fluoro-5-(1H-tetrazol-5-yl)phenyl)carbamoyl)-2-(piperidin- | 8.10 |
| | 1-yl)phenyl)-1-methyl-1H-indazole-3-carboxamide; | |
| 167 | 5-(6-(1H-tetrazol-5-yl)-2H-indazol-2-yl)-2-(piperidin-1-yl)-N-(p- | 9.41 |
| | tolyl)benzamide; | |
| 168 | N-(4-methylphenyl)-5-({[4-(5-oxo-4,5-dihydro-1,2,4-thiadiazol-3- | 14.90 |
| | yl)phenyl]methyl}amino)-2-(piperidin-1-yl)benzamide; | |
| 169 | 5-((2-fluoro-5-(1H-tetrazol-5-yl)benzyl)amino)-2-(piperidin-1-yl)-N- | 6.38 |
| | (p-tolyl)benzamide; | |
| 170 | 5-(1-oxo-5-(1H-tetrazol-5-yl)isoindolin-2-yl)-2-(piperidin-1-yl)-N- | 7.98 |
| | (p-tolyl)benzamide; and | |
| 172 | 1-ethyl-N-(5-((2-(2-(methylsulfonamido)-2- | 7.50 |
| | oxoethyl)phenyl)carbamoyl)-2-(piperidin-1-yl)phenyl)-1H-indazole- | |
| | 3-carboxamide; | |
| 173 | 2-(2-(3-(1-methyl-1H-indole-2-carboxamido)-4-(piperidin-1- | 5.85 |
| | yl)benzamido)phenyl)acetic acid | |
| 174 | 2-(2-(3-(1-methyl-1H-indazole-3-carboxamido)-4-(piperidin-1- | 3.99 |
| | yl)benzamido)phenyl)acetic acid | |
| 175 | 5-((4-(1H-tetrazol-5-yl)benzyl)amino)-2-(piperidin-1-yl)-N-(p- | 13.40 |
| | tolyl)benzamide | |
| 176 | 2-((3-((3-chloro-4-methylphenyl)carbamoyl)-4- | 5.74 |
| | (cyclopentylamino)phenyl)carbamoyl)benzoic acid | |

### Example 36: VLDL Assay Protocol

The assay was performed in black I536-well plates using purified proteins. To identify compounds that stimulate LPL activity either by directly activating LPL or by inhibiting the antagonist ANGPTL4, the LPL:sGPIHBPI complex was pre-incubated with ANGPTL4 to decrease LPL activity by 80%. After a 60 min pre-incubation, the coupled enzyme system supplemented with Amplex Red reagent was added. The enzymatic reaction was started by adding the VLDL substrate. 120 min after VLDL addition, the reaction was stopped using SDS and the fluorescence intensity was measured.

40 or 45 nl of compound solution in 100% DMSO were placed in test plate. Then LPL:sGPIHBPI was added to all wells (diluted in assay buffer to 80 nM; 1 μl/well). This was followed by an addition of ANGPTL4 to compounds and neutral controls (diluted in assay buffer to 84 nM; 1 μl/well). The assay buffer was added to stimulator control (1 μl/well). Then the plates were centrifuged and pre-incubated 60 min at RT. A NEFA (1:2 diluted in PBS) supplemented with Amplex Red (40 μM was further added to all wells (1 μl/well). The reaction was then started by adding substrate to all wells (diluted in VLDL buffer to 16 Vg/ml, 1 μl/well). The plates were further centrifuged (800 rpm for 1 my) and incubated 120 m at RT (=reaction time). The reaction was then by adding SDS to all wells (diluted in water to 2.5%, 2 μl/well) and further incubated 30 mi at RT. The fluorescence intensity (ex 540 nm/em 590 nm) was measured on a Pherastar (BMG) using 20 flashes.

Table 2 represents VLDL activation activity of cempounds of the present disclosure.

| Com- | | VLDL |
|---|---|---|
| pound | | qAC₅₀ |
| No. | Compound Name | (uM) |
| 25 | 2-(3-chloro-2-(4-(piperidin-1-yl)-3-(1-(2,2,2- | 5.0 |
| | trifluoroethyl)-1H-indazole-3- | |
| | carboxamido)benzamido)phenyl)acetic acid | |
| 30 | 2-(5-fluoro-2-(4-(piperidin-1-yl)-3-(1-(2,2,2- | 3.7 |
| | trifluoroethyl)-1H-indazole-3- | |
| | carboxamido)benzamido)phenyl)-2- | |
| | methylpropanoic acid | |
| 31 | 1-(5-fluoro-2-(4-(piperidin-1-yl)-3-(1-(2,2,2- | 3.8 |
| | trifluoroethyl)-1H-indazole-3- | |
| | carboxamido)benzamido)phenyl)cyclopropane-1- | |
| | carboxylic acid | |
| 45 | 2-(5-fluoro-2-(5-(piperidin-1-yl)-6-(1-(2,2,2- | >25 |
| | trifluoroethyl)-1H-indazole-3- | |
| | carboxamido)picolinamido)phenyl)acetic acid | |
| 63 | 2-(2-(4-(1,4-oxazepan-4-yl)-3-(1-(2,2,2- | 10 |
| | trifluoroethyl)-1H-indazole-3- | |
| | carboxamido)benzamido)-5-fluorophenyl)acetic acid | |
| 64 | 2-(5-fluoro-2-(4-(4-methylpiperidin-1-yl)-3-(1- | 1.2 |
| | (2,2,2-trifluoroethyl)-1H-indazole-3- | |
| | carboxamido)benzamido)phenyl)acetic acid | |
| 67 | 2-(5-fluoro-2-(4-(2-methylpiperidin-1-yl)-3-(1- | 2.5 |
| | (2,2,2-trifluoroethyl)-1H-indazole-3- | |
| | carboxamido)benzamido)phenyl)acetic acid | |
| 72 | 2-(2-(4-(2-ethylpiperidin-1-yl)-3-(1-(2,2,2- | 2.7 |
| | trifluoroethyl)-1H-indazole-3- | |
| | carboxamido)benzamido)-5-fluorophenyl)acetic acid | |
| 73 | 2-(5-fluoro-2-(4-(3-methylpiperidin-1-yl)-3-(1- | 2.2 |
| | (2,2,2-trifluoroethyl)-1H-indazole-3- | |
| | carboxamido)benzamido)phenyl)acetic acid | |
| 114 | 2-(5-fluoro-2-(4-(piperidin-1-yl)-3-((1-(2,2,2- | >25 |
| | trifluoroethyl)-1H-indazole)-3- | |
| | sulfonamido)benzamido)phenyl)acetic acid | |
| 120 | 2-(5-fluoro-2-(3-(1-(2-morpholinoethyl)-1H- | >25 |
| | indazole-3-carboxamido)-4-(piperidin-1- | |
| | yl)benzamido)phenyl)acetic acid | |
| 125 | 2-(5-fluoro-2-(3-(1-((5-methyl-1,2,4-oxadiazol-3- | 6.4 |
| | yl)methyl)-1H-indazole-3-carboxamido)-4- | |
| | (piperidin-1-yl)benzamido)phenyl)acetic acid | |
| 129 | 2-(5-fluoro-2-(4-(piperidin-1-yl)-3-(3-(1-(2,2,2- | 3.3 |
| | trifluoroethyl)-1H-indazol-3- | |
| | yl)ureido)benzamido)phenyl)acetic acid | |
| 132 | 2-(2-(3-(1-((1,4-dioxan-2-yl)methyl)-1H-indazole- | 7.4 |
| | 3-carboxamido)-4-(piperidin-1-yl)benzamido)-5- | |
| | fluorophenyl)acetic acid | |

### Example 37: RB Assay Protocol

The assay was performed in black 1536-well plates using purified proteins. To identify compounds that stimulate LPL activity either by directly activating LPL or by inhibiting the antagonist ANGPTL4, the LPL:sGPIHBP1 complex was pre-incubated with ANGPTL4 to decrease LPL activity by 80%. After a 60 min pre-incubation, the reaction was started by adding the RB substrate. 30 min after RB addition, the enzymatic reaction was stopped using SDS. Fluorescence intensity was measured 2 h after stopping the reaction.

20 or 40 nl of compound solution in 100% DMSO were placed in test plate. Then was added LPL:sGPIHBP1 to all wells (diluted in assay buffer to 5 nM; 2 μl/well). This was followed by an addition of ANGPTL4 to compounds and neutral controls (diluted in assay buffer to 20 nM; 1 μl/well). A buffer solution was added to the stimulator control (1 μl/well). The plates were then centrifuged and pre-incubated for 60 min at RT. The reaction was started by adding substrate to all wells (diluted in assay buffer to 40 μM, 1 μl/well). The plated were centrifuged (800 rpm for 1 min) and incubated 30 min at RT (=reaction time). The reaction was stopped by adding SDS to all wells (diluted in water to 2.5%, 2 μl/well); incubated 2 h at RT. The fluorescence intensity (ex 540 nm/em 590 nm) was measured on a Pherastar (BMG) using 5 flashes.

Table 3 represents RB activation activity of compounds of the present disclosure.

| Com- | | RB |
|---|---|---|
| pound | | qAC₅₀ |
| No. | Compound Name | (uM) |
| 1 | 2-(5-fluoro-2-(N-methyl-4-(piperidin-1-yl)-3-(1- | >25 |
| | (2,2,2-trifluoroethyl)-1H-indazole-3- | |
| | carboxamido)benzamido)phenyl)acetic acid | |
| 3 | 5-(2-(4-(piperidin-1-yl)-3-(1-(2,2,2-trifluoroethyl)- | >25 |
| | 1H-indazole-3-carboxamido)benzamido)-2- | |
| | fluorophenyl)acetic acid | |
| 4 | 2-(2-(4-(piperidin-1-yl)-3-(1-(2,2,2-trifluoroethyl)- | 2.6 |
| | 1H-indazole-3- | |
| | carboxamido)benzamido)phenyl)acetic acid | |
| 5 | 2-(4-(piperidin-1-yl)-3-(1-(2,2,2-trifluoroethyl)- | 4.6 |
| | 1H-indazole-3-carboxamido)benzamido)benzoic acid | |
| 6 | 2-(5-fluoro-2-(4-(piperidin-1-yl)-3-(1-(2,2,2- | 6.0 |
| | trifluoroethyl)-1H-indazole-3- | |
| | carboxamido)benzamido)phenyl)propanoic acid | |
| 9 | 3-(2-(4-(piperidin-1-yl)-3-(1-(2,2,2-trifluoroethyl)- | 24 |
| | 1H-indazole-3- | |
| | carboxamido)benzamido)phenyl)propanoic acid | |
| 10 | 3-(4-(piperidin-1-yl)-3-(1-(2,2,2-trifluoroethyl)- | >25 |
| | 1H-indazole-3-carboxamido)benzamido)benzoic acid | |
| 11 | 4-(4-(piperidin-1-yl)-3-(1-(2,2,2-trifluoroethyl)- | >25 |
| | 1H-indazole-3-carboxamido)benzamido)benzoic acid | |
| 16 | N-(5-((2-(2-amino-2-oxoethyl)phenyl)carbamoyl)- | >25 |
| | 2-(piperidin-1-yl)phenyl)-1-(2,2,2-trifluoroethyl)- | |
| | 1H-indazole-3-carboxamide | |
| 17 | 2-(4-(piperidin-1-yl)-3-(1-(2,2,2-trifluoroethyl)- | >25 |
| | 1H-indazole-3-carboxamido)benzamido)nicotinic acid | |
| 18 | 2-((4-(piperidin-1-yl)-3-(1-(2,2,2-trifluoroethyl)- | 11 |
| | 1H-indazole-3- | |
| | carboxamido)benzamido)methyl)benzoic acid | |
| 19 | 3-((4-(piperidin-1-yl)-3-(1-(2,2,2-trifluoroethyl)- | >7.1 |
| | 1H-indazole-3- | |
| | carboxamido)benzamido)methyl)benzoic acid | |
| 20 | 2-(5-fluoro-2-((4-(piperidin-1-yl)-3-(1-(2,2,2- | >7.1 |
| | trifluoroethyl)-1H-indazole-3- | |
| | carboxamido)phenyl)sulfonamido)phenyl)acetic acid | |
| 22 | 2-(2-(4-(piperidin-1-yl)-3-(1-(2,2,2-trifluoroethyl)- | 4.3 |
| | 1H-indazole-3-carboxamido)benzamido)-5- | |
| | (trifluoromethyl)phenyl)acetic acid | |
| 23 | 2-(5-chloro-2-(4-(piperidin-1-yl)-3-(1-(2,2,2- | 4.6 |
| | trifluoroethyl)-1H-indazole-3- | |
| | carboxamido)benzamido)phenyl)acetic acid | |
| 24 | 2-(3-fluoro-2-(4-(piperidin-1-yl)-3-(1-(2,2,2- | 3.2 |
| | trifluoroethyl)-1H-indazole-3- | |
| | carboxamido)benzamido)phenyl)acetic acid | |
| 26 | 3-chloro-2-(4-(piperidin-1-yl)-3-(1-(2,2,2- | 4.5 |
| | trifluoroethyl)-1H-indazole-3- | |
| | carboxamido)benzamido)benzoic acid | |
| 27 | 3-fluoro-2-(4-(piperidin-1-yl)-3-(1-(2,2,2- | 7.1 |
| | trifluoroethyl)-1H-indazole-3- | |
| | carboxamido)benzamido)benzoic acid | |
| 28 | 2-(5-fluoro-2-(4-(piperidin-1-yl)-3-(1-(2,2,2- | 4.2 |
| | trifluoroethyl)-1H-indazole-3- | |
| | carboxamido)benzamido)phenyl)butanoic acid | |
| 33 | 2-(5-methyl-2-(4-(piperidin-1-yl)-3-(1-(2,2,2- | 3.6 |
| | trifluoroethyl)-1H-indazole-3- | |
| | carboxamido)benzamido)phenyl)acetic acid | |
| 37 | 2-(2-fluoro-6-(4-(piperidin-1-yl)-3-(1-(2,2,2- | 5.0 |
| | trifluoroethyl)-1H-indazole-3- | |
| | carboxamido)benzamido)phenyl)acetic acid | |
| 38 | 2-(4-fluoro-2-(4-(piperidin-1-yl)-3-(1-(2,2,2- | 3.6 |
| | trifluoroethyl)-1H-indazole-3- | |
| | carboxamido)benzamido)phenyl)acetic acid | |
| 39 | 2-(2-(4-(azetidin-1-yl)-3-(1-(2,2,2-trifluoroethyl)- | >25 |
| | 1H-indazole-3-carboxamido)benzamido)-5- | |
| | fluorophenyl)acetic acid | |
| 40 | 2-(2-(4-(4,4-difluoropiperidin-1-yl)-3-(1-(2,2,2- | 3.5 |
| | trifluoroethyl)-1H-indazole-3- | |
| | carboxamido)benzamido)-5-fluorophenyl)acetic acid | |
| 41 | 2-(5-fluoro-2-(4-(piperidin-1-yl)-3-((1-(2,2,2- | 4.8 |
| | trifluoroethyl)-1H-indazol-3- | |
| | yl)carbamoyl)benzamido)phenyl)acetic acid | |
| 42 | 2-(5-fluoro-2-(2-methyl-4-(piperidin-1-yl)-3-(1- | >25 |
| | (2,2,2-trifluoroethyl)-1H-indazole-3- | |
| | carboxamido)benzamido)phenyl)acetic acid | |
| 43 | 2-(5-fluoro-2-(5-(piperidin-1-yl)-4-(1-(2,2,2- | >7.1 |
| | trifluoroethyl)-1H-indazole-3- | |
| | carboxamido)picolinamido)phenyl)acetic acid | |
| 44 | 2-(5-fluoro-2-(6-(piperidin-1-yl)-5-(1-(2,2,2- | >25 |
| | trifluoroethyl)-1H-indazole-3- | |
| | carboxamido)nicotinamido)phenyl)acetic acid | |
| 48 | 2-(2-(4-(4-cyanopiperidin-1-yl)-3-(1-(2,2,2- | >25 |
| | trifluoroethyl)-1H-indazole-3- | |
| | carboxamido)benzamido)-5-fluorophenyl)acetic acid | |
| 49 | 2-(2-(4-(2-azaspiro[3.3]heptan-2-yl)-3-(1-(2,2,2- | 20 |
| | trifluoroethyl)-1H-indazole-3- | |
| | carboxamido)benzamido)-5-fluorophenyl)acetic acid | |
| 50 | 2-(5-fluoro-2-(4-(pyrrolidin-1-yl)-3-(1-(2,2,2- | 24 |
| | trifluoroethyl)-1H-indazole-3- | |
| | carboxamido)benzamido)phenyl)acetic acid | |
| 51 | 2-(2-((4-(piperidin-1-yl)-3-((1-(2,2,2- | 4.1 |
| | trifluoroethyl)-1H-indazol-3- | |
| | yl)carbamoyl)phenyl)carbamoyl)phenyl)acetic acid | |
| 52 | 2-(2-(4-(3-azabicyclo[3.1.0]hexan-3-yl)-3-(1- | 5.0 |
| | (2,2,2-trifluoroethyl)-1H-indazole-3- | |
| | carboxamido)benzamido)-5-fluorophenyl)acetic acid | |
| 53 | 2-(2-(4-(5-azaspiro[2.4]heptan-5-yl)-3-(1-(2,2,2- | 6.8 |
| | trifluoroethyl)-1H-indazole-3- | |
| | carboxamido)benzamido)-5-fluorophenyl)acetic acid | |
| 54 | 2-(2-(4-((1s,4s)-7-azabicyclo[2.2.1]heptan-7-yl)-3- | 6.9 |
| | (1-(2,2,2-trifluoroethyl)-1H-indazole-3- | |
| | carboxamido)benzamido)-5-fluorophenyl)acetic acid | |
| 55 | 2-(5-fluoro-2-(2-methyl-4-(piperidin-1-yl)-5-(1- | 5.0 |
| | (2,2,2-trifluoroethyl)-1H-indazole-3- | |
| | carboxamido)benzamido)phenyl)acetic acid | |
| 56 | 2-(5-fluoro-2-(4-morpholino-3-(1-(2,2,2- | 15 |
| | trifluoroethyl)-1H-indazole-3- | |
| | carboxamido)benzamido)phenyl)acetic acid | |
| 57 | 2-(5-fluoro-2-(4-(4-methylpiperazin-1-yl)-3-(1- | >7.1 |
| | (2,2,2-trifluoroethyl)-1H-indazole-3- | |
| | carboxamido)benzamido)phenyl)acetic acid | |
| 58 | rac 2-(2-(4-(2-azabicyclo[2.2.1]heptan-2-yl)-3-(1- | 18 |
| | (2,2,2-trifluoroethyl)-1H-indazole-3- | |
| | carboxamido)benzamido)-5-fluorophenyl)acetic acid | |
| 59 | 2-(5-fluoro-2-(4-(2-oxopiperidin-1-yl)-3-(1-(2,2,2- | >25 |
| | trifluoroethyl)-1H-indazole-3- | |
| | carboxamido)benzamido)phenyl)acetic acid | |
| 60 | 2-(5-fluoro-2-(4-(2-oxooxazolidin-3-yl)-3-(1- | >25 |
| | (2,2,2-trifluoroethyl)-1H-indazole-3- | |
| | carboxamido)benzamido)phenyl)acetic acid | |
| 61 | 2-(2-(4-(4,4-dimethylpiperidin-1-yl)-3-(1-(2,2,2- | 3.6 |
| | trifluoroethyl)-1H-indazole-3- | |
| | carboxamido)benzamido)-5-fluorophenyl)acetic acid | |
| 62 | 2-(2-(4-(3,3-difluoropiperidin-1-yl)-3-(1-(2,2,2- | >7.1 |
| | trifluoroethyl)-1H-indazole-3- | |
| | carboxamido)benzamido)-5-fluorophenyl)acetic acid | |
| 65 | 2-(5-fluoro-2-(4-(4-methoxypiperidin-1-yl)-3-(1- | >7.1 |
| | (2,2,2-trifluoroethyl)-1H-indazole-3- | |
| | carboxamido)benzamido)phenyl)acetic acid | |
| 66 | 2-(2-(4-(1,1-dioxidothiomorpholino)-3-(1-(2,2,2- | >25 |
| | trifluoroethyl)-1H-indazole-3- | |
| | carboxamido)benzamido)-5-fluorophenyl)acetic acid | |
| 68 | 2-(2-(4-(2,6-dimethylpiperidin-1-yl)-3-(1-(2,2,2- | >7.1 |
| | trifluoroethyl)-1H-indazole-3- | |
| | carboxamido)benzamido)-5-fluorophenyl)acetic acid | |
| 71 | 2-(2-(4-(2-carbamoylpiperidin-1-yl)-3-(1-(2,2,2- | >7.1 |
| | trifluoroethyl)-1H-indazole-3- | |
| | carboxamido)benzamido)-5-fluorophenyl)acetic acid | |
| 74 | 2-(2-(4-(3,5-dimethylpiperidin-1-yl)-3-(1-(2,2,2- | >7.1 |
| | trifluoroethyl)-1H-indazole-3- | |
| | carboxamido)benzamido)-5-fluorophenyl)acetic acid | |
| 75 | 2-(5-fluoro-2-(4-(4-hydroxypiperidin-1-yl)-3-(1- | >25 |
| | (2,2,2-trifluoroethyl)-1H-indazole-3- | |
| | carboxamido)benzamido)phenyl)acetic acid | |
| 94 | 2-(2-(3-(1-(cyclopropylmethyl)-1H-indazole-3- | 5.9 |
| | carboxamido)-4-(piperidin-1-yl)benzamido)-5- | |
| | fluorophenyl)acetic acid | |
| 95 | 2-(5-fluoro-2-(3-(1-isobutyl-1H-indazole-3- | 5.0 |
| | carboxamido)-4-(piperidin-1- | |
| | yl)benzamido)phenyl)acetic acid | |
| 96 | 2-(2-(3-(1-(2,2-difluoroethyl)-1H-indazole-3- | 4.7 |
| | carboxamido)-4-(piperidin-1-yl)benzamido)-5- | |
| | fluorophenyl)acetic acid | |
| 97 | 2-(2-(3-(1-cyclopropyl-1H-indazole-3- | 13 |
| | carboxamido)-4-(piperidin-1-yl)benzamido)-5- | |
| | fluorophenyl)acetic acid | |
| 98 | 2-(2-(3-(1-(cyclobutylmethyl)-1H-indazole-3- | 6.1 |
| | carboxamido)-4-(piperidin-1-yl)benzamido)-5- | |
| | fluorophenyl)acetic acid | |
| 99 | 2-(5-fluoro-2-(3-(1-(2-hydroxyethyl)-1H-indazole- | >25 |
| | 3-carboxamido)-4-(piperidin-1- | |
| | yl)benzamido)phenyl)acetic acid | |
| 100 | 2-(2-(3-(1-(2-aminoethyl)-1H-indazole-3- | >25 |
| | carboxamido)-4-(piperidin-1-yl)benzamido)-5- | |
| | fluorophenyl)acetic acid | |
| 101 | 2-(5-fluoro-2-(4-(piperidin-1-yl)-3-(1-(2- | >25 |
| | (pyrrolidin-1-yl)ethyl)-1H-indazole-3- | |
| | carboxamido)benzamido)phenyl)acetic acid | |
| 102 | 2-(2-(3-(1-(cyanomethyl)-1H-indazole-3- | 5.1 |
| | carboxamido)-4-(piperidin-1-yl)benzamido)-5- | |
| | fluorophenyl)acetic acid | |
| 103 | 2-(5-fluoro-2-(4-(piperidin-1-yl)-3-(1-(piperidin-4- | >25 |
| | ylmethyl)-1H-indazole-3- | |
| | carboxamido)benzamido)phenyl)acetic acid | |
| 104 | 2-(5-fluoro-2-(4-(piperidin-1-yl)-3-(1-((tetrahydro- | 3.2 |
| | 2H-pyran-4-yl)methyl)-1H-indazole-3- | |
| | carboxamido)benzamido)phenyl)acetic acid | |
| 105 | 2-(5-fluoro-2-(3-(1-(2-methoxyethyl)-1H-indazole- | 14 |
| | 3-carboxamido)-4-(piperidin-1- | |
| | yl)benzamido)phenyl)acetic acid | |
| 106 | 2-(5-fluoro-2-(3-(1-(2-hydroxy-2-methylpropyl)- | 7.7 |
| | 1H-indazole-3-carboxamido)-4-(piperidin-1- | |
| | yl)benzamido)phenyl)acetic acid | |
| 107 | 2-(5-fluoro-2-(4-(piperidin-1-yl)-3-(1-(2,2,2- | >7.1 |
| | trifluoroethyl)-1H-pyrazolo[4,3-c]pyridine-3- | |
| | carboxamido)benzamido)phenyl)acetic acid | |
| 109 | 2-(5-fluoro-2-(3-(imidazo[1,2-a]pyridine-2- | >25 |
| | carboxamido)-4-(piperidin-1- | |
| | yl)benzamido)phenyl)acetic acid | |
| 110 | 2-(5-fluoro-2-(3-(1-methylimidazo[1,5-a]pyridine- | >7.1 |
| | 3-carboxamido)-4-(piperidin-1- | |
| | yl)benzamido)phenyl)acetic acid | |
| 111 | 2-(5-fluoro-2-(4-(piperidin-1-yl)-3-(1-(2,2,2- | 23 |
| | trifluoroethyl)-1H-pyrazolo[4,3-b]pyridine-3- | |
| | carboxamido)benzamido)phenyl)acetic acid | |
| 112 | 2-(5-fluoro-2-(4-(piperidin-1-yl)-3-(1-(2,2,2- | 6.4 |
| | trifluoroethyl)-1H-pyrazolo[3,4-c]pyridine-3- | |
| | carboxamido)benzamido)phenyl)acetic acid | |
| 113 | 2-(5-fluoro-2-(4-(piperidin-1-yl)-3-(1-(2,2,2- | 9.0 |
| | trifluoroethyl)-1H-pyrazolo[3,4-b]pyridine-3- | |
| | carboxamido)benzamido)phenyl)acetic acid | |
| 115 | 2-(5-fluoro-2-(3-(4-phenyl-1-(2,2,2-trifluoroethyl)- | >7.1 |
| | 1H-pyrazole-3-carboxamido)-4-(piperidin-1- | |
| | yl)benzamido)phenyl)acetic acid | |
| 116 | 2-(5-fluoro-2-(3-(5-phenyl-1-(2,2,2-trifluoroethyl)- | 23 |
| | 1H-pyrazole-3-carboxamido)-4-(piperidin-1- | |
| | yl)benzamido)phenyl)acetic acid | |
| 117 | 2-(2-(3-(4-cyclopropyl-1-(2,2,2-trifluoroethyl)-1H- | >25 |
| | pyrazole-3-carboxamido)-4-(piperidin-1- | |
| | yl)benzamido)-5-fluorophenyl)acetic acid | |
| 118 | 2-(5-fluoro-2-(4-(piperidin-1-yl)-3-(1- | >7.1 |
| | ((tetrahydrofuran-3-yl)methyl)-1H-indazole-3- | |
| | carboxamido)benzamido)phenyl)acetic acid | |
| 119 | 2-(5-fluoro-2-(4-(piperidin-1-yl)-3-(1- | 6.6 |
| | ((tetrahydrofuran-2-yl)methyl)-1H-indazole-3- | |
| | carboxamido)benzamido)phenyl)acetic acid | |
| 121 | 2-(5-fluoro-2-(4-(piperidin-1-yl)-3-(1-(3,3,3- | 4.2 |
| | trifluoropropyl)-1H-indazole-3- | |
| | carboxamido)benzamido)phenyl)acetic acid | |
| 122 | 2-(5-fluoro-2-(4-(piperidin-1-yl)-3-(1-(prop-2-yn- | 3.8 |
| | 1-yl)-1H-indazole-3- | |
| | carboxamido)benzamido)phenyl)acetic acid | |
| 123 | 2-(2-(3-(1-(but-2-yn-1-yl)-1H-indazole-3- | 3.1 |
| | carboxamido)-4-(piperidin-1-yl)benzamido)-5- | |
| | fluorophenyl)acetic acid | |
| 124 | 2-(5-fluoro-2-(3-(1-((5-methylisoxazol-3- | 4.7 |
| | yl)methyl)-1H-indazole-3-carboxamido)-4- | |
| | (piperidin-1-yl)benzamido)phenyl)acetic acid | |
| 127 | 2-(2-(3-(1-((1-cyanocyclopropyl)methyl)-1H- | 4.3 |
| | indazole-3-carboxamido)-4-(piperidin-1- | |
| | yl)benzamido)-5-fluorophenyl)acetic acid | |
| 128 | 2-(5-fluoro-2-(4-(piperidin-1-yl)-3-(1-(2,2,2- | 0.66 |
| | trifluoroethyl)-1H-indole-3- | |
| | carboxamido)benzamido)phenyl)acetic acid | |
| 130 | 2-(5-fluoro-2-(3-(2-methyl-1-(2,2,2-trifluoroethyl)- | >25 |
| | 1H-indole-3-carboxamido)-4-(piperidin-1- | |
| | yl)benzamido)phenyl)acetic acid | |
| 133 | 2-(5-fluoro-2-(4-(piperidin-1-yl)-3-(1-((tetrahydro- | 4.2 |
| | 2H-pyran-3-yl)methyl)-1H-indazole-3- | |
| | carboxamido)benzamido)phenyl)acetic acid | |
| 134 | 2-(5-fluoro-2-(3-(3-phenyl-1-(2,2,2-trifluoroethyl)- | 4.4 |
| | 1H-pyrazole-5-carboxamido)-4-(piperidin-1- | |
| | yl)benzamido)phenyl)acetic acid | |
| 135 | 2-(5-fluoro-2-(4-(piperidin-1-yl)-3-(2-(2,2,2- | 12 |
| | trifluoroethyl)-2H-indazole-3- | |
| | carboxamido)benzamido)phenyl)acetic acid | |
| | hydrochloride | |
| 136 | (S)-2-(5-fluoro-2-(4-(piperidin-1-yl)-3-(1- | >25 |
| | ((tetrahydrofuran-2-yl)methyl)-1H-indazole-3- | |
| | carboxamido)benzamido)phenyl)acetic acid | |
| 137 | (R)-2-(5-fluoro-2-(4-(piperidin-1-yl)-3-(1- | 3.8 |
| | ((tetrahydrofuran-2-yl)methyl)-1H-indazole-3- | |
| | carboxamido)benzamido)phenyl)acetic acid | |
| 138 | 2-(5-fluoro-2-(3-(5-fluoro-1-(2,2,2-trifluoroethyl)- | 4.8 |
| | 1H-indazole-3-carboxamido)-4-(piperidin-1- | |
| | yl)benzamido)phenyl)acetic acid | |
| 141 | 2-(5-fluoro-2-(3-(4-methyl-1-(2,2,2-trifluoroethyl)- | >25 |
| | 1H-indazole-3-carboxamido)-4-(piperidin-1- | |
| | yl)benzamido)phenyl)acetic acid | |
| 142 | 2-(5-fluoro-2-(3-(5-methyl-1-(2,2,2-trifluoroethyl)- | >7.1 |
| | 1H-indazole-3-carboxamido)-4-(piperidin-1- | |
| | yl)benzamido)phenyl)acetic acid | |
| 143 | 2-(5-fluoro-2-(3-(6-methyl-1-(2,2,2-trifluoroethyl)- | 5.0 |
| | 1H-indazole-3-carboxamido)-4-(piperidin-1- | |
| | yl)benzamido)phenyl)acetic acid | |
| 144 | 2-(5-fluoro-2-(3-(7-methyl-1-(2,2,2-trifluoroethyl)- | 5.8 |
| | 1H-indazole-3-carboxamido)-4-(piperidin-1- | |
| | yl)benzamido)phenyl)acetic acid | |
| 145 | 2-(5-fluoro-2-(4-(piperidin-1-yl)-3-(1-(2,2,2- | 15 |
| | trifluoroethyl)-1,4,5,6- | |
| | tetrahydrocyclopenta[c]pyrazole-3- | |
| | carboxamido)benzamido)phenyl)acetic acid | |
| 146 | 2-(5-fluoro-2-(4-(piperidin-1-yl)-3-(1-(1,1,1- | >7.1 |
| | trifluoropropan-2-yl)-1H-indazole-3- | |
| | carboxamido)benzamido)phenyl)acetic acid | |
| 148 | 2-(5-fluoro-2-(4-(piperidin-1-yl)-3-(1-(1- | >25 |
| | (tetrahydro-2H-pyran-4-yl)ethyl)-1H-indazole-3- | |
| | carboxamido)benzamido)phenyl)acetic acid | |

### Example 38: Activity of LPL Activators in Cellular Lipolysis Assay and Angptl4 Protection Assay

Chinese hamster ovary (CHO) mutant pgsA-745 cells were grown in F-12K media (ATCC) with 10% FBS and penicillin/streptomycin. One day prior to transfection 300,000 cells were seeded per well in a 6 well plate and cultured overnight. 2 μg of human GPIHBPI in pcDNA3.1(+)—C-HA (Genscript) were transfected using Lipofectamine 3000 (Invitrogen) and the cells were cultured for 24 hours. Cells were split into selection media (600 μg/ml G418 (Gibco)) and a stable pool was established and verified by qPCR and western blotting using anti-HA (Cell Signaling) and GPIHBP1 (Santa Cruz) antibodies (data not shown).

CHO pgsA-745 cells overexpressing GPIHBPI were seeded at 200,000 cells per well in 24 well plates and cultured overnight. Media was replaced with serum-free F-12K media containing 0.1 U/ml heparin and cultured for 2 hours to remove surface bound endogenous LPL, then cells were washed with PBS. Human LPL (R&D Systems) was diluted to 50 nM in serum free media and added for 30 minutes gently shaking at room temperature, then cells were washed with PBS to remove unbound LPL. Test compounds were diluted in serum free media and added to cells for 20 minutes at room temperature gently shaking, followed by 10× ANGPTL4 (30 nM final concentration) for another 20 minutes. Test compound and ANGPTL4 were removed and cells washed with PBS. Serum free media was added to the cells followed by 5× EnzChek substrate (Invitrogen, 1 mM stock) diluted 1:67 in 25 mM EPPS, 125 mM NaCl, 0.03% triton, 0.03% fatty acid free BSA (final concentration 3 uM) and the cells were incubated for 40 minutes at 37° C. Media was removed and fluorescence read at Ex482/Em515. For intrinsic activation studies Angptl4 was omitted and the compound incubated for 40 minutes.

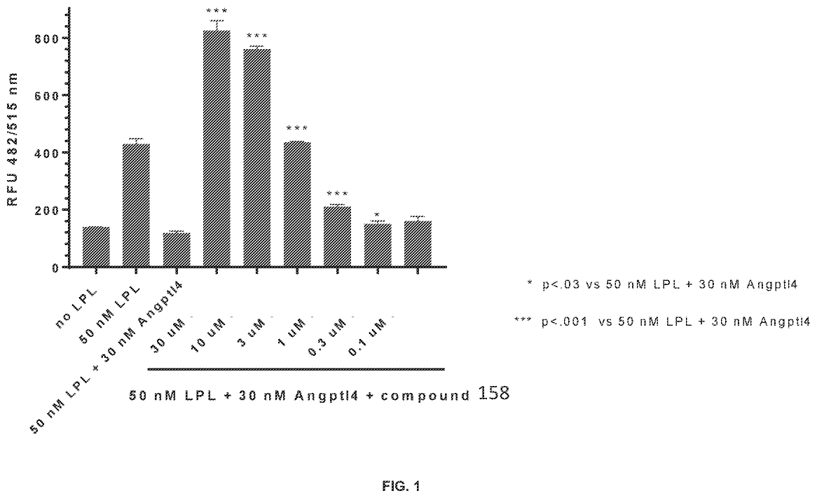

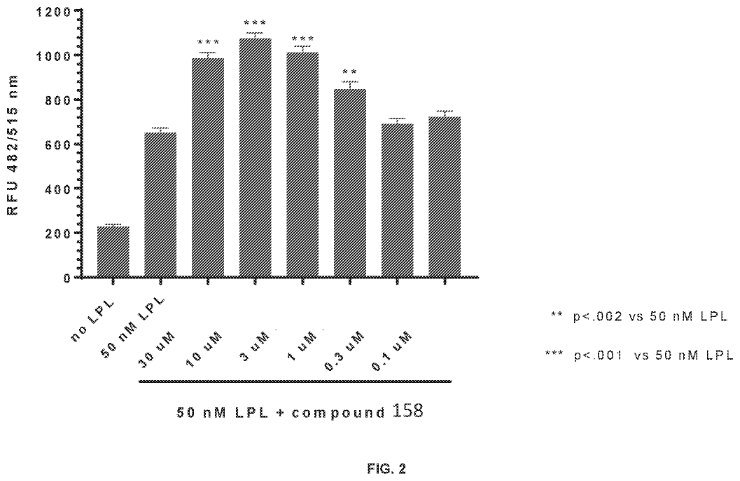

As shown in FIG. 1, addition of exogenous LPL increased activity, while addition of Angptl4 lowered activity as determined by fluorescent readout of the EnzChek substrate that produces a fluorescent product in the presence of lipases. As shown in FIG. 2, addition of Compound 158 significantly increased lipolysis activity at 30 μM, 10 μM, 3 μM, 1 M and 0.3 μM doses as compared to addition of 50 nM LPL with 30 nM Angptl4. For the intrinsic activation studies, addition of Compound 158 significantly increased lipolysis activity at 30 μM, 10 PM, 3 μM, and 1 μM doses as compared to addition of 50 nM LPL alone. Together this data suggests that Compound 158 activates LPL even in the presence of Angptl4, an inactivator of LPL.

### Example 39: LPL Effect on Postprandial Triglyceridaemia

To examine to effects of test compounds on postprandial triglycerdemia, 5-7 week old C₅₇BL/6 mice were fasted overnight (18 hours) on non-nutritive bedding (Alpha-Dri). Following the overnight fast, the mice are bled via retro-orbital sinus (25 μL) using microcapillaries (t-1 hr sample). The LPL activator test compound was then administered intraperitoneally (IP) in a dose volume of 10 ml/kg. One hour later the mice were bled for the t=0 timepoint. The mice were then immediately dosed with a 10 mL/kg oral bolus of olive oil. The mice were subsequently bled at 1-, 2-, and 3.5-hours post-olive oil administration.

Whole blood samples were collected into tubes containing 0.125% EDTA solution in saline. Diluted plasma samples were analyzed for triglyceride levels using Infinity Triglyceride reagent (Thermo) on a microtiter plate. Briefly, 10 μL of diluted sample was added the plate. 200 μL of the reagent was added and the plate was incubated for 10 minutes at 37° C. The plate was then read at 550 nM on a Molecular Devices M5 Spectrophotometer (San Jose, CA). The concentration of triglycerides in the sample was determined using a standard curve made using a dilution of triglyceride standard (Sigma-Aldrich, St. Louis, MO) using the Softmax software from Molecular Devices. All graphing and subsequent area under-the-curve and statistical analysis (Student's t Test) is performed using GraphPad Prism (version 8.0, GraphPad Software, Inc., San Diego, CA).

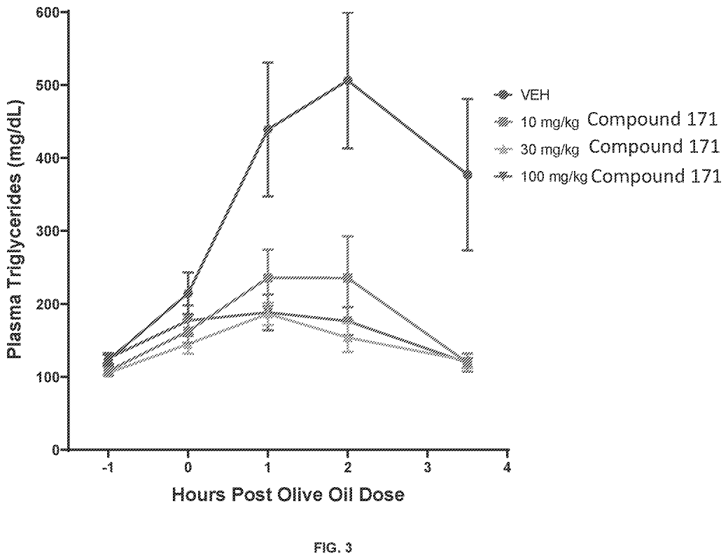

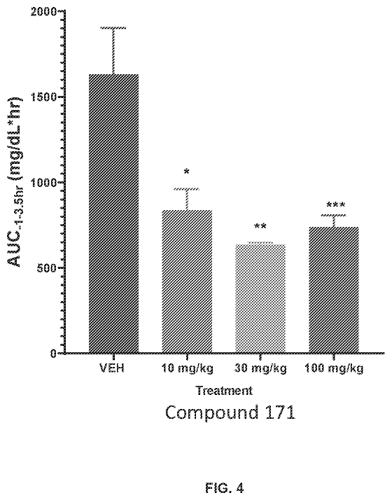

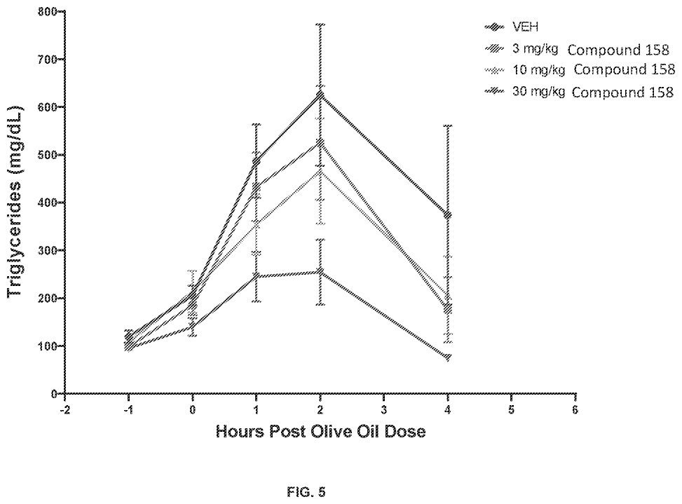

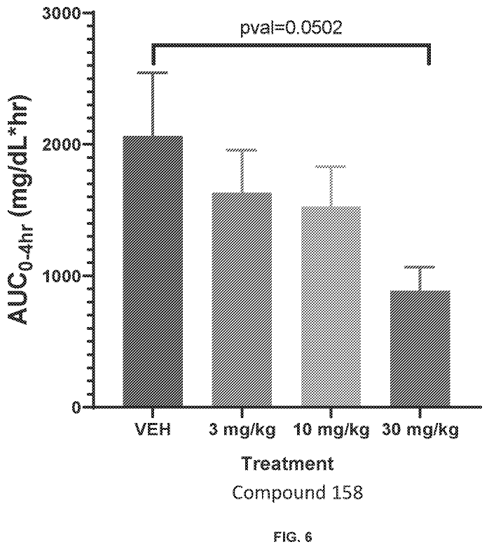

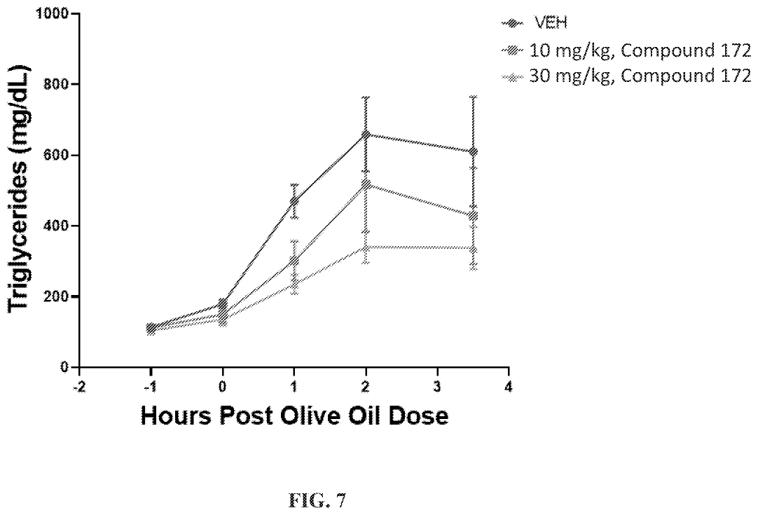

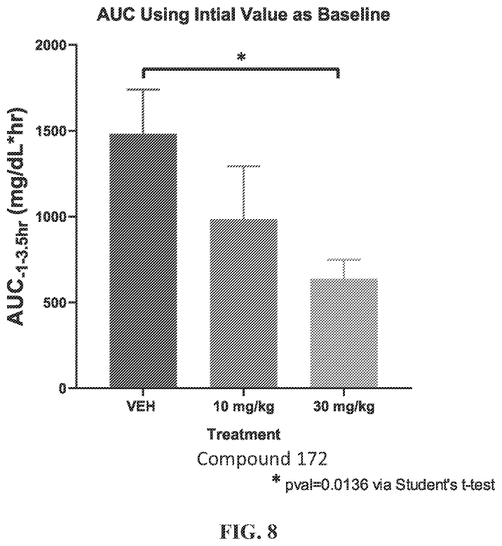

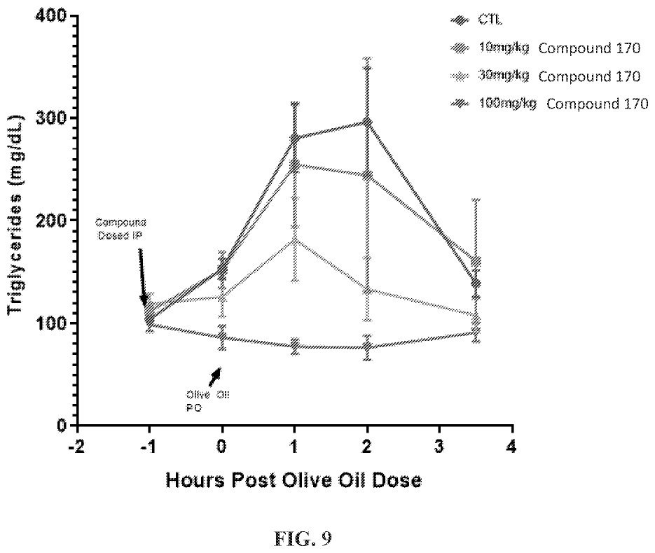

As shown in FIGS. 3-9, test compounds 158 and 170-172 blunt hypertriglyceridemia response to oral olive oil gavage in a dose dependent fashion. Taken together, these results suggest that the compounds may have significant effects on post-prandial triglyceride-rich lipoprotein excursion, and portend improved post-prandial triglyceridaemia profiles in humans, which has been demonstrated to be an important risk factor for CVD.

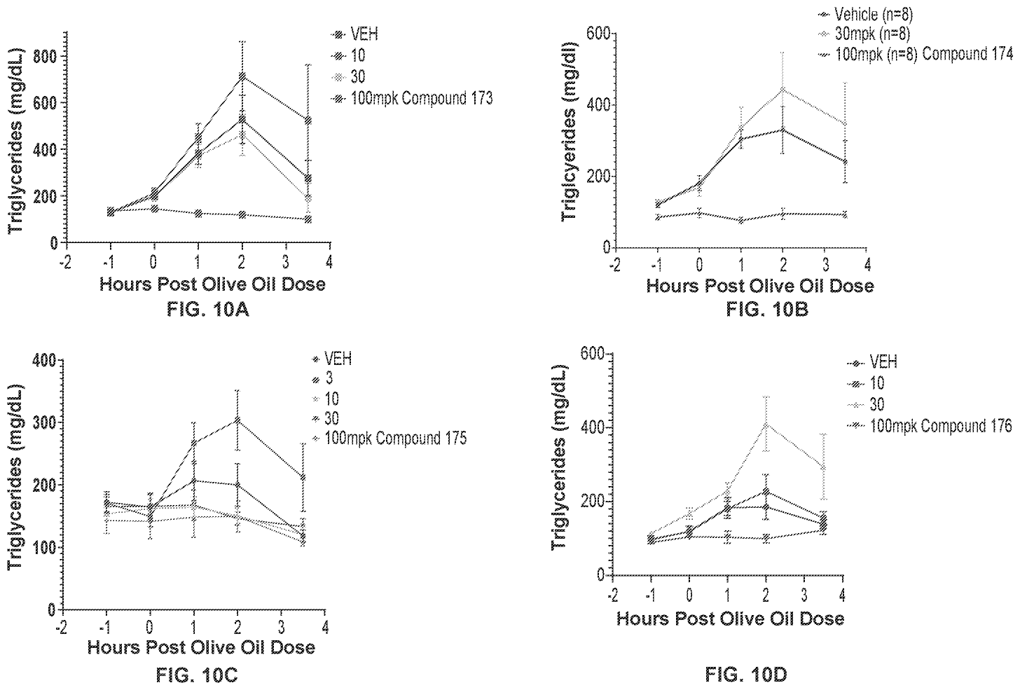

As shown in FIGS. 10A-10D, test compounds 173-176 blunt hypertriglyceridemia response to oral olive oil gavage in a dose dependent fashion. Taken together, these results suggest that the compounds may have significant effects on post-prandial triglyceride-rich lipoprotein excursion, and portend improved post-prandial triglyceridemia profiles in humans, which has been demonstrated to be an important risk factor for CVD.

### Example 40: In Vivo Effects on Fasting Triglycerides

In vivo effects of the test compounds on fasting triglycerides in an apoC-IIT hypertriglyceridemia mouse model will be examined. Briefly, C57B16 male mice aged ˜56d at 20-25g will be selected. Mice will be housed 5 per cage in a 12h day/night cycle and fed standard rodent chow and water ad libitum. For over expression of human apoC-III to drive elevated fasting triglyceride levels, mice will be injected IP with an adeno-associated viral vector (thyroxine binding globulin (TBG) promoter, serotype 8 (liver-directed)). Fourteen days following administration of 6×10¹¹ genome copies per mouse, all mice will be 6h fasted and bled via retro-orbital sinus. Plasma triglyceride levels will be determined. Plasma hApoC-III levels will be quantitated using an enzyme-linked immunosorbent assay (ELISA) for human ApoC-III. Sixteen mice will be selected and assigned to 2 groups to achieve equivalent starting mean hApoC-III levels and triglyceride levels. A cohort of naïve animals will be used as a normal control and also bled for baseline triglyceride levels.

At t-12 hours at the beginning of the dark cycle, hApoC-III animals will be administered test compound at a dose of 25 mg/kg in 2.5% DMSO/10% Solutol in 0.9% saline via a single intraperitoneal dose at 10 ml/kg dose volume. An additional hApoC-III animals will be administered vehicle via intraperitoneal injection. The cohort of naïve animals will be administered vehicle via intraperitoneal injection. At t=0 all of the animals will be bled via retro-orbital sinus. All food will be removed and animals will be returned to cages with free access to water. Blood samples will be taken via retro-orbital sinus at 1, 2, 4, 6, and 8 hours. All food will be returned at the conclusion of the study. Data will be plotted and analyzed, and individual AUC's using 0 (zero) as the baseline will be calculated. Statistical significance will be determined using ANOVA with a Tukey's multiple comparison post-test. All statistical analyses were performed using GraphPad Prism software (version 8.0, GraphPad Software, Inc., San Diego, CA).

It is contemplated that treatment with test compound will return fasted triglyceride levels of hAAV8-APOC3 transgenic mice to wild type levels and that treatment will significantly reduce the triglyceride AUC measured from 12 to 20 hours post-dose compared to vehicle treatment, with an AUC reduced to the level observed in the naïve (wild-type) mice with normal fasting triglyceride levels.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula I: 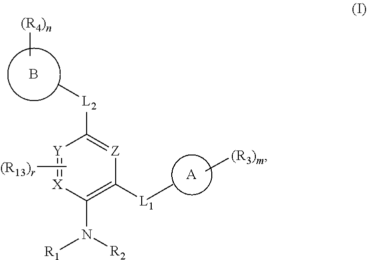
or a pharmaceutically acceptable salt, isomer, solvate, prodrug, or tautomer thereof, wherein:
X, Y, and Z are independently CH or N;
Ring A is heteroaryl;
Ring B is selected from aryl and heteroaryl;
L₁ is selected from —N(R₇)—C(O)—N(R₇)—, —(CH₂)ₚ—N(R₇)—C(O)—, —C(O)—N(R₇)—(CH₂)ₚ—, —NR, —S(O)₂—, and —S(O)₂—NR₇—;
L₂ is selected from —(CH₂)ₚ—N(R₇)—C(O)—, —C(O)—N(R₇HCH₂)ₚ—, —NR₇—CH₂—, —CH₂—NR₇—, —OCH₂—, —CH₂O—, —NR₇—S(O)₂—, —S(O)₂—NR₇—, —N(R₇)—NH₂—, or NH₂—N(R₇)—;
R₁ and R₂are taken together with the N atom to which they are attached to form 3- to 10-membered heterocyclyl, wherein the heterocyclyl is optionally substituted with one or more R₅;
each R₃ is independently selected from halogen, —CN, —OH, —C(O)CH₃, C₁-C₆ alkyl, C₁-C₆ alkoxy, C₂-C₆ alkynyl, C₃-C₁₀ cycloalkyl, and C₆-C₁₀ aryl, wherein the alkyl, alkoxy, alkynyl cycloalkyl, or aryl is optionally substituted with one or more R₆;
each R₄ is independently selected from halogen, —OH, —[C(R₁₀)₂]ₒ—COOH, —{C(R₁₀)₂]ₒ—CON(R₁₁)₂, C₁-C₆ alkyl, C₃-C₁₀ cycloalkyl, and 5- to 10-membered heteroaryl, wherein the alkyl, cycloalkyl, and the heteroaryl is optionally substituted with one or more groups selected from halogen, C₁-C₆ alkyl, —OH, and oxo;
each R₁₀ is independently halogen, C₁-C₆ alkyl, or C₃-C₁₀ carbocyclyl, or
two R₁₀ are taken together to form an C₃-C₆ cycloalkyl;
each R₁₁ is independently hydrogen, C₁-C₆ alkyl, or —S(═O)₂R₁₂;
R₁₂ is C₁-C₆ alkyl or C₃-C₁₀ cycloalkyl;
each R₅ is independently selected from oxo, —OH, halogen, —C(═O)NH₂, —C(═O)OH, —CN, C₁-C₆ alkyl, C₁-C₆ alkoxy, C₁-C₆ haloalkyl, C₃-C₁₀ cycloalkyl, and aryl; wherein the alkyl, alkoxy, haloalkyl, cycloalkyl, and aryl is optionally substituted with one or more R₈; or
two R₅, together with the intervening atoms, forms a C₃-C₁₀ cycloalkyl or 3- to 10-membered heterocyclyl;
R₆ is independently selected from halogen, —OH, —NH₂, —CN, C₁-C₆ alkyl, C₂-C₆ alkynyl, C₁-C₆ alkoxy, C₁-C₆ haloalkoxy, C₁-C₆ haloalkyl, C₃-C₁₀ cycloalkyl, 3- to 10-membered heterocyclyl, heteroaryl, and C₆-C₁₀ aryl, wherein the cycloalkyl, heteroaryl or aryl is optionally substituted with one or more R₁₃;
R₇ is selected from H and C₁-C₃ alkyl; or
when L₂ is —(CH₂)ₚ—N(R₇)—C(O)—, —C(O)—N(R₇)—(CH₂)ₚ—, —N(R₇)—NH₂—, or —NH₂—N(R₇)—, and at least one R₄ is ortho to the L₂ substituent, R₄ and R₇, together with the atoms to which they are attached, combine to form a 5- to 6-membered heterocyclyl or heteroaryl;
each R₈ is independently selected from —OH, —NH₂, and —C(═O)NH₂;
each R₁₃ is independently selected from halogen, —OH, —NH₂, —CN, C₁-C₆ alkyl, C₁-C₆ alkoxy, C₁-C₆ haloalkoxy, C₁-C₆ haloalkyl, C₃-C₁₀ cycloalkyl, 3- to 10-membered heterocyclyl, heteroaryl, and C₆-C₁₀ aryl;
m is an integer selected from 0, 1, 2, 3, 4, 5, and 6;
n is an integer selected from 0, 1, 2, 3, 4, and 5;
o is an integer selected from 0 to 2;
p is 0 or 1;
r is an integer selected from 0 to 3.

2. A compound of Formula I: 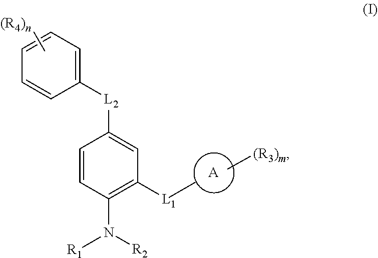
or a pharmaceutically acceptable salt, isomer, solvate, prodrug, or tautomer thereof, wherein:
Ring A is 5- to 10-membered heteroaryl;
L₁ is selected from —NH—C(O)— and —C(O)—NH—;
L₂ is selected from —NH—C(O)—, —C(O)—NH—, —N(R₇)—C(O)—, —C(O)—N(R₇)—, —NH—CH₂—, —CH₂—NH—, —N(R₇)—NH₂—, or —NH₂—N(R₇)—;
R₁ and R₂ are taken together with the N atom to which they are attached to form 3- to 10-membered heterocyclyl;
each R₃ is independently selected from halogen, C₁-C₆ alkyl, and C₆-C₁₀ aryl, wherein the alkyl is optionally substituted with one or more R₆;
each R₄ is independently selected from halogen, —(CH₂)ₒ-COOH, and 5- to 10-membered heteroaryl, wherein the heteroaryl is optionally substituted with oxo;
R₆ is independently selected from halogen and C₃-C₁₀ aryl;
R₇ is selected from H and C₁-C₃ alkyl; or
when L₂ is —N(R₇)—C(O)—, —C(O)—N(R₂)—, —N(R₇)—NH₂—, or —NH₂—N(R₇)—, and at least one R₄ is ortho to the L₂ substituent, R₄ and R₇, together with the atoms to which they are attached, combine to form a 5- to 6-membered heterocyclyl or heteroaryl;
m is an integer selected from 0, 1, and 2;
n is an integer selected from 0, 1, and 2; and
o is 1;
wherein when one R₄ and R₇, together with the atoms to which they are attached, combine to form a 5- to 6-membered heterocyclyl or heteroaryl, then n is 2, 3, 4 or 5 and no R₄ is —(CH₂)ₒ—COOH

3. The compound of any one of the previous claims, wherein the compound is of Formula (Ia): 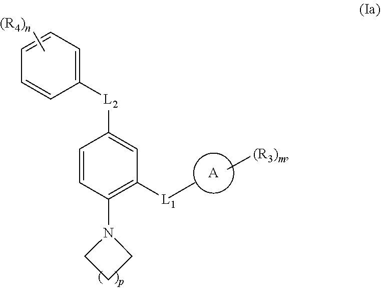
or a pharmaceutically acceptable salt, isomer, solvate, prodrug, or tautomer thereof, wherein p is an integer selected from 0, 1, 2, 3, 4, 5, 6, and 7.

4. The compound of any one of the previous claims, wherein the compound is of Formula (Ia-i): 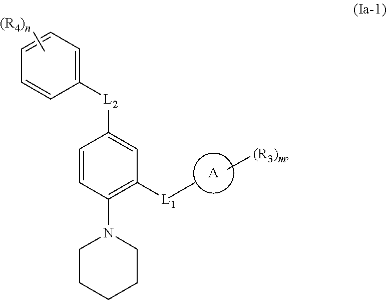
or a pharmaceutically acceptable salt, isomer, solvate, prodrug, or tautomer thereof.

5. The compound of any one of the previous claims, wherein the compound is of Formula (Ib-1), (Ib-2), (Ib-3), (Ib-4), or (Ib-5): 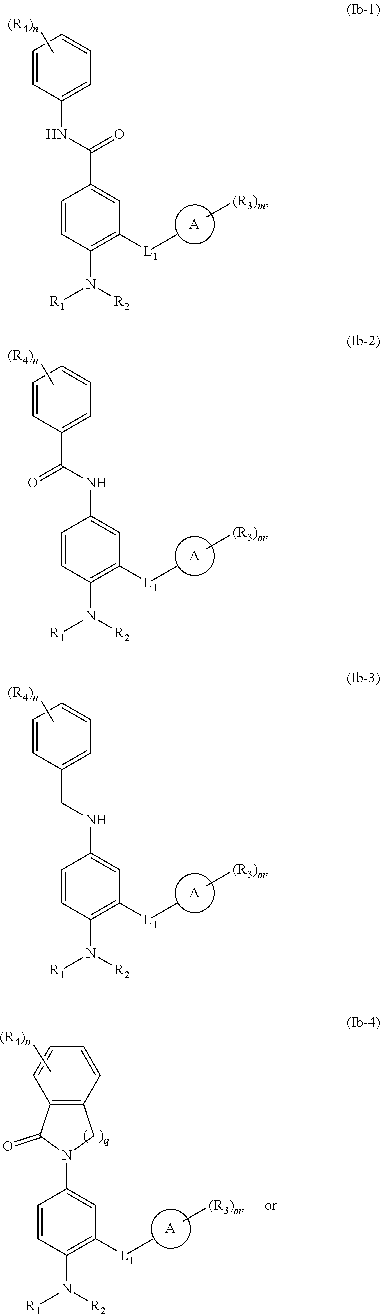 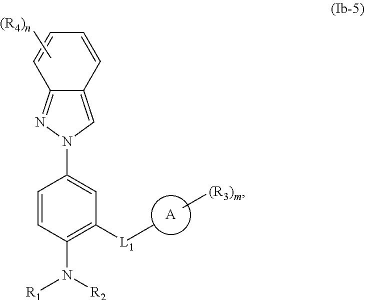
or a pharmaceutically acceptable salt, isomer, solvate, prodrug, or tautomer thereof, wherein q is an integer selected from 1 and 2.

6. The compound of any one of the previous claims, wherein the compound is of Formula (Ic-1) or (Ic-2): 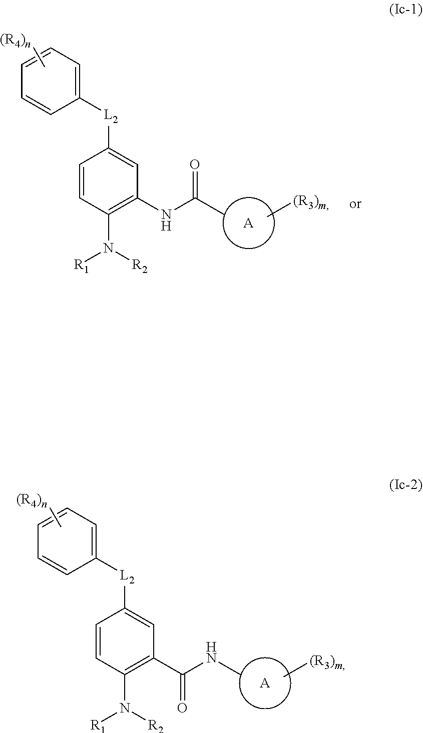
or a pharmaceutically acceptable salt, isomer, solvate, prodrug, or tautomer thereof.

7. The compound of any one of the previous claims, wherein the compound is of Formula (Id-1), (Id-2), (Id-3), (Id-4), (Id-5), (Id-6), or (Id-7): 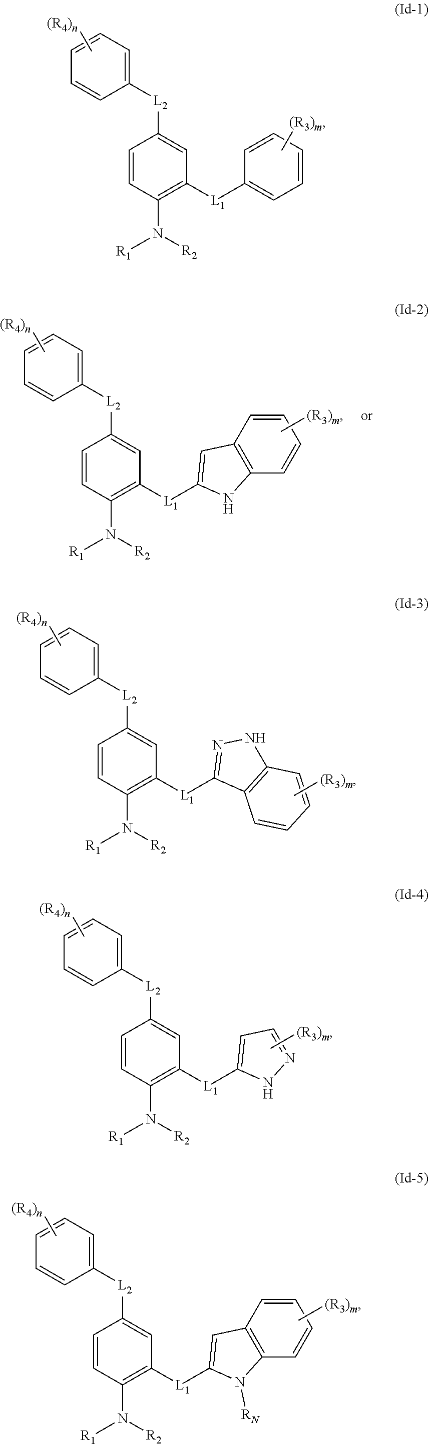 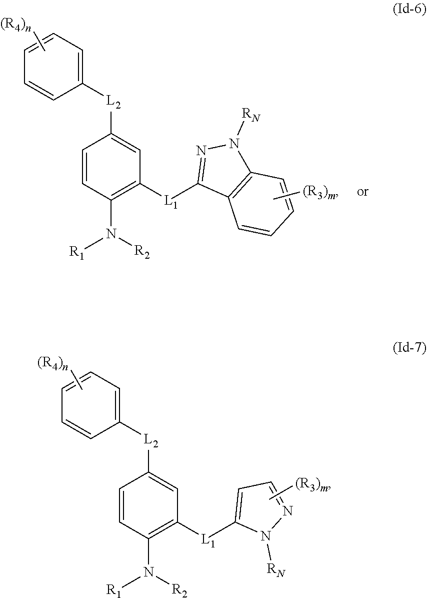
or a pharmaceutically acceptable salt, isomer, solvate, prodrug, or tautomer thereof; wherein R_{N} is selected from C₁-C₆ alkyl, wherein the alkyl is optionally substituted with one or more halo or aryl.

8. The compound of any one of the previous claims, wherein the compound is of Formulae (Ie-I) or (Ie-2): 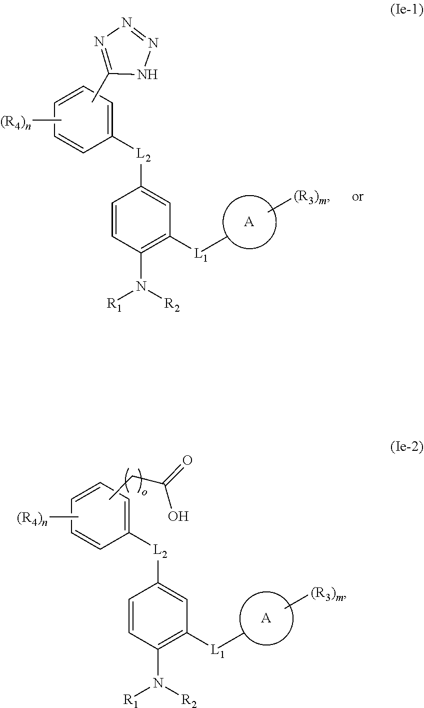
or a pharmaceutically acceptable salt, isomer, solvate, prodrug, or tautomer thereof.

9. The compound of any one of the previous claims, wherein L₂ is selected from —NH—C(O)— and —C(O)—NH—.

10. The compound of any one of the previous claims, wherein L₂ is selected from —NH—CH₂—and —CH₂—NH—.

11. The compound of any one of the previous claims, wherein L₂ is —N(R₇)—C(O)—, —C(O)—N(R₇)—, —N(R₇)—NH₂—, or —NH₂—N(R₇)—, at least one R₄ is ortho to the L₂ substituent, and R₄ and R₇, together with the atoms to which they are attached, combine to form a 5- to 6-membered heterocyclyl or heteroaryl.

12. The compound of any one of the previous claims, wherein Ring A is heteroaryl.

13. The compound of any one of the previous claims, wherein Ring A is bicyclic.

14. The compound of any one of the previous claims, wherein Ring A is selected from

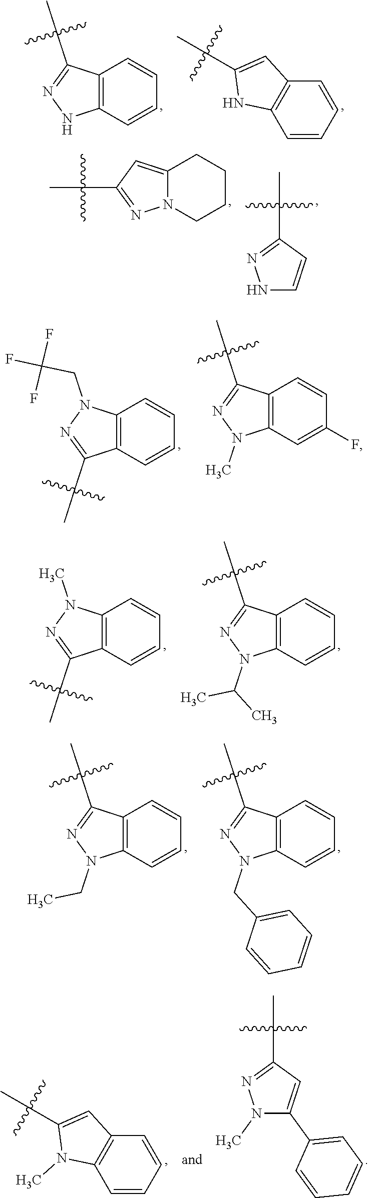

15. The compound ofany one of the previous claims, wherein at least one R₃ is C₁-C₆ alkyl.

16. The compound of any one of the previous claims, wherein at least one R₃ is C₁-C₆ alkyl substituted with one or more R₆.

17. The compound of any one of the previous claims, wherein at least one R₃ is C₁-C₆ alkyl substituted with one or more halogen.

18. The compound of any one of the previous claims, wherein at least one R₃ is C₁-C₆ alkyl substituted with one or more C₆-C₁₀ aryl.

19. The compound of any one of the previous claims, wherein at least one R₃ is methyl.

20. The compound of any one of the previous claims, wherein at least one R₃ is halogen.

21. The compound of any one of the previous claims, wherein at least one R₃ is C₆-C₁₀ aryl.

22. The compound of any one of the previous claims, wherein R₃ is selected from —F, —Cl, —CH₃,

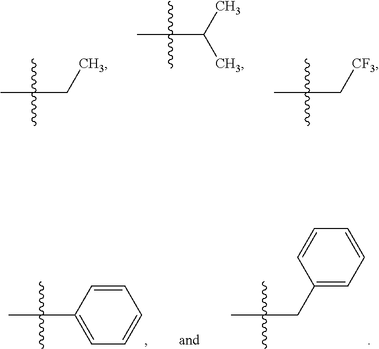

23. The compound of any one of the previous claims, wherein at least one R is 5- to 10-membered heteroaryl.

24. The compound of any one of the previous claims, wherein at least one R is tetrazole.

25. The compound of any one of the previous claims, wherein at least one R₄ is —CH₂—COOH.

26. The compound of any one of the previous claims, wherein at least one R₄ is halogen.

27. The compound of any one of the previous claims, wherein at least one R₄ is fluoro.

28. The compound of any one of the previous claims, wherein at least one R₄ is fluoro and at least one R₄ is —CH₂—COOH.

29. The compound of any one of the previous claims, wherein R₄ is selected from —CH₂—COOH,

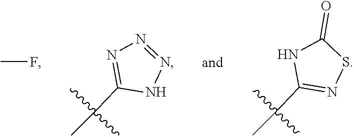

30. The compound of any one of the previous claims, wherein m is 0.

31. The compound of any one of the previous claims, wherein m is 1.

32. The compound of any one of the previous claims, wherein m is 2.

33. The compound of any one of the previous claims, wherein n is 0.

34. The compound of any one of the previous claims, wherein n is 1.

35. The compound of any one of the previous claims, wherein n is 2.

36. The compound of any one of the previous claims, wherein o is 1.

37. The compound of any one of the previous claims, wherein the compound is selected from:

| Compound # | Name |
|---|---|
| 1 | 2-(5-fluoro-2-(N-methyl-4-(piperidin-1-yl)-3-(1-(2,2,2-trifluoroethyl)-1H- |
| | indazole-3-carboxamido)benzamido)phenyl)acetic acid |
| 2 | 4-(4-(piperidin-1-yl)-3-(1-(2,2,2-trifluoroethyl)-1H-indazole-3- |
| | carboxamido)benzamido)nicotinic acid |
| 3 | 5-(2-(4-(piperidin-1-yl)-3-(1-(2,2,2-trifluoroethyl)-1H-indazole-3- |
| | carboxamido)benzamido)-2-fluorophenyl)acetic acid |
| 4 | 2-(2-(4-(piperidin-1-yl)-3-(1-(2,2,2-trifluoroethyl)-1H-indazole-3- |
| | carboxamido)benzamido)phenyl)acetic acid |
| 5 | 2-(4-(piperidin-1-yl)-3-(1-(2,2,2-trifluoroethyl)-1H-indazole-3- |
| | carboxamido)benzamido)benzoic acid |
| 6 | 2-(5-fluoro-2-(4-(piperidin-1-yl)-3-(1-(2,2,2-trifluoroethyl)-1H-indazole-3- |
| | carboxamido)benzamido)phenyl)propanoic acid |
| 7 | 2-fluoro-2-(5-fluoro-2-(4-(piperidin-1-yl)-3-(1-(2,2,2-trifluoroethyl)-1H- |
| | indazole-3-carboxamido)benzamido)phenyl)acetic acid |
| 7a | 2,2-difluoro-2-(5-fluoro-2-(4-(piperidin-1-yl)-3-(1-(2,2,2-trifluoroethyl)-1H- |
| | indazole-3-carboxamido)benzamido)phenyl)acetic acid |
| 8 | 2,2-difluoro-2-(2-(4-(piperidin-1-yl)-3-(1-(2,2,2-trifluoroethyl)-1H-indazole-3- |
| | carboxamido)benzamido)phenyl)acetic acid |
| 9 | 3-(2-(4-(piperidin-1-yl)-3-(1-(2,2,2-trifluoroethyl)-1H-indazole-3- |
| | carboxamido)benzamido)phenyl)propanoic acid |
| 10 | 3-(4-(piperidin-1-yl)-3-(1-(2,2,2-trifluoroethyl)-1H-indazole-3- |
| | carboxamido)benzamido)benzoic acid |
| 11 | 4-(4-(piperidin-1-yl)-3-(1-(2,2,2-trifluoroethyl)-1H-indazole-3- |
| | carboxamido)benzamido)benzoic acid |
| 11a | 2-(4-(4-(piperidin-1-yl)-3-(1-(2,2,2-trifluoroethyl)-1H-indazole-3- |
| | carboxamido)benzamido)phenyl)acetic acid |
| 12 | 2-(3-fluoro-4-(4-(piperidin-1-yl)-3-(1-(2,2,2-trifluoroethyl)-1H-indazole-3- |
| | carboxamido)benzamido)phenyl)acetic acid |
| 13 | N-(5-((4-fluoro-2-(2-(methylsulfonamido)-2-oxoethyl)phenyl)carbamoyl)-2- |
| | (piperidin-1-yl)phenyl)-1-(2,2,2-trifluoroethyl)-1H-indazole-3-carboxamide |
| 14 | N-(5-((2-(2-(ethylsulfonamido)-2-oxoethyl)-4-fluorophenyl)carbamoyl)-2- |
| | (piperidin-1-yl)phenyl)-1-(2,2,2-trifluoroethyl)-1H-indazole-3-carboxamide |
| 15 | N-(5-((2-(2-(cyclopropanesulfonamido)-2-oxoethyl)-4-fluorophenyl)carbamoyl)- |
| | 2-(piperidin-1-yl)phenyl)-1-(2,2,2-trifluoroethyl)-1H-indazole-3-carboxamide |
| 16 | N-(5-((2-(2-amino-2-oxoethyl)phenyl)carbamoyl)-2-(piperidin-1-yl)phenyl)-1- |
| | (2,2,2-trifluoroethyl)-1H-indazole-3-carboxamide |
| 16a | 2-(2-(4-(piperidin-1-yl)-3-(1-(2,2,2-trifluoroethyl)-1H-indazole-3- |
| | carboxamido)benzamido)pyridin-3-yl)acetic acid |
| 17 | 2-(4-(piperidin-1-yl)-3-(1-(2,2,2-trifluoroethyl)-1H-indazole-3- |
| | carboxamido)benzamido)nicotinic acid |
| 18 | 2-((4-(piperidin-1-yl)-3-(1-(2,2,2-trifluoroethyl)-1H-indazole-3- |
| | carboxamido)benzamido)methyl)benzoic acid |
| 19 | 3-((4-(piperidin-1-yl)-3-(1-(2,2,2-trifluoroethyl)-1H-indazole-3- |
| | carboxamido)benzamido)methyl)benzoic acid |
| 20 | 2-(5-fluoro-2-((4-(piperidin-1-yl)-3-(1-(2,2,2-trifluoroethyl)-1H-indazole-3- |
| | carboxamido)phenyl)sulfonamido)phenyl)acetic acid |
| 21 | 2-(5-fluoro-2-((N-methyl-4-(piperidin-1-yl)-3-(1-(2,2,2-trifluoroethyl)-1H- |
| | indazole-3-carboxamido)phenyl)sulfonamido)phenyl)acetic acid |
| 22 | 2-(2-(4-(piperidin-1-yl)-3-(1-(2,2,2-trifluoroethyl)-1H-indazole-3- |
| | carboxamido)benzamido)-5-(trifluoromethyl)phenyl)acetic acid |
| 23 | 2-(5-chloro-2-(4-(piperidin-1-yl)-3-(1-(2,2,2-trifluoroethyl)-1H-indazole-3- |
| | carboxamido)benzamido)phenyl)acetic acid |
| 24 | 2-(3-fluoro-2-(4-(piperidin-1-yl)-3-(1-(2,2,2-trifluoroethyl)-1H-indazole-3- |
| | carboxamido)benzamido)phenyl)acetic acid |
| 25 | 2-(3-chloro-2-(4-(piperidin-1-yl)-3-(1-(2,2,2-trifluoroethyl)-1H-indazole-3- |
| | carboxamido)benzamido)phenyl)acetic acid |
| 26 | 3-chloro-2-(4-(piperidin-1-yl)-3-(1-(2,2,2-trifluoroethyl)-1H-indazole-3- |
| | carboxamido)benzamido)benzoic acid |
| 27 | 3-fluoro-2-(4-(piperidin-1-yl)-3-(1-(2,2,2-trifluoroethyl)-1H-indazole-3- |
| | carboxamido)benzamido)benzoic acid |
| 28 | 2-(5-fluoro-2-(4-(piperidin-1-yl)-3-(1-(2,2,2-trifluoroethyl)-1H-indazole-3- |
| | carboxamido)benzamido)phenyl)butanoic acid |
| 29 | 2-cyclopropyl-2-(5-fluoro-2-(4-(piperidin-1-yl)-3-(1-(2,2,2-trifluoroethyl)-1H- |
| | indazole-3-carboxamido)benzamido)phenyl)acetic acid |
| 30 | 2-(5-fluoro-2-(4-(piperidin-1-yl)-3-(1-(2,2,2-trifluoroethyl)-1H-indazole-3- |
| | carboxamido)benzamido)phenyl)-2-methylpropanoic acid |
| 31 | 1-(5-fluoro-2-(4-(piperidin-1-yl)-3-(1-(2,2,2-trifluoroethyl)-1H-indazole-3- |
| | carboxamido)benzamido)phenyl)cyclopropane-1-carboxylic acid |
| 32 | 2-(2-methyl-6-(4-(piperidin-1-yl)-3-(1-(2,2,2-trifluoroethyl)-1H-indazole-3- |
| | carboxamido)benzamido)phenyl)acetic acid |
| 33 | 2-(5-methyl-2-(4-(piperidin-1-yl)-3-(1-(2,2,2-trifluoroethyl)-1H-indazole-3- |
| | carboxamido)benzamido)phenyl)acetic acid |
| 34 | 2-(4-methyl-2-(4-(piperidin-1-yl)-3-(1-(2,2,2-trifluoroethyl)-1H-indazole-3- |
| | carboxamido)benzamido)phenyl)acetic acid |
| 35 | 2-(3-methyl-2-(4-(piperidin-1-yl)-3-(1-(2,2,2-trifluoroethyl)-1H-indazole-3- |
| | carboxamido)benzamido)phenyl)acetic acid |
| 36 | 2-(5-cyclopropyl-2-(4-(piperidin-1-yl)-3-(1-(2,2,2-trifluoroethyl)-1H-indazole-3- |
| | carboxamido)benzamido)phenyl)acetic acid |
| 37 | 2-(2-fluoro-6-(4-(piperidin-1-yl)-3-(1-(2,2,2-trifluoroethyl)-1H-indazole-3- |
| | carboxamido)benzamido)phenyl)acetic acid |
| 38 | 2-(4-fluoro-2-(4-(piperidin-1-yl)-3-(1-(2,2,2-trifluoroethyl)-1H-indazole-3- |
| | carboxamido)benzamido)phenyl)acetic acid |
| 39 | 2-(2-(4-(azetidin-1-yl)-3-(1-(2,2,2-trifluoroethyl)-1H-indazole-3- |
| | carboxamido)benzamido)-5-fluorophenyl)acetic acid |
| 40 | 2-(2-(4-(4,4-difluoropiperidin-1-yl)-3-(1-(2,2,2-trifluoroethyl)-1H-indazole-3- |
| | carboxamido)benzamido)-5-fluorophenyl)acetic acid |
| 41 | 2-(5-fluoro-2-(4-(piperidin-1-yl)-3-((1-(2,2,2-trifluoroethyl)-1H-indazol-3- |
| | yl)carbamoyl)benzamido)phenyl)acetic acid |
| 42 | 2-(5-fluoro-2-(2-methyl-4-(piperidin-1-yl)-3-(1-(2,2,2-trifluoroethyl)-1H- |
| | indazole-3-carboxamido)benzamido)phenyl)acetic acid |
| 43 | 2-(5-fluoro-2-(5-(piperidin-1-yl)-4-(1-(2,2,2-trifluoroethyl)-1H-indazole-3- |
| | carboxamido)picolinamido)phenyl)acetic acid |
| 44 | 2-(5-fluoro-2-(6-(piperidin-1-yl)-5-(1-(2,2,2-trifluoroethyl)-1H-indazole-3- |
| | carboxamido)nicotinamido)phenyl)acetic acid |
| 45 | 2-(5-fluoro-2-(5-(piperidin-1-yl)-6-(1-(2,2,2-trifluoroethyl)-1H-indazole-3- |
| | carboxamido)picolinamido)phenyl)acetic acid |
| 46 | 1-(4-((2-(carboxymethyl)-4-fluorophenyl)carbamoyl)-2-(1-(2,2,2-trifluoroethyl)- |
| | 1H-indazole-3-carboxamido)phenyl)piperidine-4-carboxylic acid |
| 47 | 2-(5-fluoro-2-(4-(piperazin-1-yl)-3-(1-(2,2,2-trifluoroethyl)-1H-indazole-3- |
| | carboxamido)benzamido)phenyl)acetic acid |
| 48 | 2-(2-(4-(4-cyanopiperidin-1-yl)-3-(1-(2,2,2-trifluoroethyl)-1H-indazole-3- |
| | carboxamido)benzamido)-5-fluorophenyl)acetic acid |
| 49 | 2-(2-(4-(2-azaspiro[3.3]heptan-2-yl)-3-(1-(2,2,2-trifluoroethyl)-1H-indazole-3- |
| | carboxamido)benzamido)-5-fluorophenyl)acetic acid |
| 50 | 2-(5-fluoro-2-(4-(pyrrolidin-1-yl)-3-(1-(2,2,2-trifluoroethyl)-1H-indazole-3- |
| | carboxamido)benzamido)phenyl)acetic acid |
| 51a | 2-(5-fluoro-2-((4-(piperidin-1-yl)-3-((1-(2,2,2-trifluoroethyl)-1H-indazol-3- |
| | yl)carbamoyl)phenyl)carbamoyl)phenyl)acetic acid |
| 51 | 2-(2-((4-(piperidin-1-yl)-3-((1-(2,2,2-trifluoroethyl)-1H-indazol-3- |
| | yl)carbamoyl)phenyl)carbamoyl)phenyl)acetic acid |
| 52 | 2-(2-(4-(3-azabicyclo[3.1.0]hexan-3-yl)-3-(1-(2,2,2-trifluoroethyl)-1H-indazole- |
| | 3-carboxamido)benzamido)-5-fluorophenyl)acetic acid |
| 53 | 2-(2-(4-(5-azaspiro[2.4]heptan-5-yl)-3-(1-(2,2,2-trifluoroethyl)-1H-indazole-3- |
| | carboxamido)benzamido)-5-fluorophenyl)acetic acid |
| 54 | 2-(2-(4-((1s,4s)-7-azabicyclo[2.2.1]heptan-7-yl)-3-(1-(2,2,2-trifluoroethyl)-1H- |
| | indazole-3-carboxamido)benzamido)-5-fluorophenyl)acetic acid |
| 55 | 2-(5-fluoro-2-(2-methyl-4-(piperidin-1-yl)-5-(1-(2,2,2-trifluoroethyl)-1H- |
| | indazole-3-carboxamido)benzamido)phenyl)acetic acid |
| 56 | 2-(5-fluoro-2-(4-morpholino-3-(1-(2,2,2-trifluoroethyl)-1H-indazole-3- |
| | carboxamido)benzamido)phenyl)acetic acid |
| 57 | 2-(5-fluoro-2-(4-(4-methylpiperazin-1-yl)-3-(1-(2,2,2-trifluoroethyl)-1H- |
| | indazole-3-carboxamido)benzamido)phenyl)acetic acid |
| 58 | rac 2-(2-(4-(2-azabicyclo[2.2.1]heptan-2-yl)-3-(1-(2,2,2-trifluoroethyl)-1H- |
| | indazole-3-carboxamido)benzamido)-5-fluorophenyl)acetic acid |
| 59 | 2-(5-fluoro-2-(4-(2-oxopiperidin-1-yl)-3-(1-(2,2,2-trifluoroethyl)-1H-indazole-3- |
| | carboxamido)benzamido)phenyl)acetic acid |
| 60 | 2-(5-fluoro-2-(4-(2-oxooxazolidin-3-yl)-3-(1-(2,2,2-trifluoroethyl)-1H-indazole- |
| | 3-carboxamido)benzamido)phenyl)acetic acid |
| 61 | 2-(2-(4-(4,4-dimethylpiperidin-1-yl)-3-(1-(2,2,2-trifluoroethyl)-1H-indazole-3- |
| | carboxamido)benzamido)-5-fluorophenyl)acetic acid |
| 62 | 2-(2-(4-(3,3-difluoropiperidin-1-yl)-3-(1-(2,2,2-trifluoroethyl)-1H-indazole-3- |
| | carboxamido)benzamido)-5-fluorophenyl)acetic acid |
| 63 | 2-(2-(4-(1,4-oxazepan-4-yl)-3-(1-(2,2,2-trifluoroethyl)-1H-indazole-3- |
| | carboxamido)benzamido)-5-fluorophenyl)acetic acid |
| 64 | 2-(5-fluoro-2-(4-(4-methylpiperidin-1-yl)-3-(1-(2,2,2-trifluoroethyl)-1H- |
| | indazole-3-carboxamido)benzamido)phenyl)acetic acid |
| 65 | 2-(5-fluoro-2-(4-(4-methoxypiperidin-1-yl)-3-(1-(2,2,2-trifluoroethyl)-1H- |
| | indazole-3-carboxamido)benzamido)phenyl)acetic acid |
| 66 | 2-(2-(4-(1,1-dioxidothiomorpholino)-3-(1-(2,2,2-trifluoroethyl)-1H-indazole-3- |
| | carboxamido)benzamido)-5-fluorophenyl)acetic acid |
| 67 | 2-(5-fluoro-2-(4-(2-methylpiperidin-1-yl)-3-(1-(2,2,2-trifluoroethyl)-1H- |
| | indazole-3-carboxamido)benzamido)phenyl)acetic acid |
| 68 | 2-(2-(4-(2,6-dimethylpiperidin-1-yl)-3-(1-(2,2,2-trifluoroethyl)-1H-indazole-3- |
| | carboxamido)benzamido)-5-fluorophenyl)acetic acid |
| 69 | 2-(5-fluoro-2-(4-(2-(hydroxymethyl)piperidin-1-yl)-3-(1-(2,2,2-trifluoroethyl)- |
| | 1H-indazole-3-carboxamido)benzamido)phenyl)acetic acid |
| 70 | 2-(2-(4-(2-(aminomethyl)piperidin-1-yl)-3-(1-(2,2,2-trifluoroethyl)-1H-indazole- |
| | 3-carboxamido)benzamido)-5-fluorophenyl)acetic acid |
| 71 | 2-(2-(4-(2-carbamoylpiperidin-1-yl)-3-(1-(2,2,2-trifluoroethyl)-1H-indazole-3- |
| | carboxamido)benzamido)-5-fluorophenyl)acetic acid |
| 72 | 2-(2-(4-(2-ethylpiperidin-1-yl)-3-(1-(2,2,2-trifluoroethyl)-1H-indazole-3- |
| | carboxamido)benzamido)-5-fluorophenyl)acetic acid |
| 73 | 2-(5-fluoro-2-(4-(3-methylpiperidin-1-yl)-3-(1-(2,2,2-trifluoroethyl)-1H- |
| | indazole-3-carboxamido)benzamido)phenyl)acetic acid |
| 74 | 2-(2-(4-(3,5-dimethylpiperidin-1-yl)-3-(1-(2,2,2-trifluoroethyl)-1H-indazole-3- |
| | carboxamido)benzamido)-5-fluorophenyl)acetic acid |
| 75 | 2-(5-fluoro-2-(4-(4-hydroxypiperidin-1-yl)-3-(1-(2,2,2-trifluoroethyl)-1H- |
| | indazole-3-carboxamido)benzamido)phenyl)acetic acid |
| 76 | 2-(2-(4-(2-oxa-6-azaspiro[3.3]heptan-6-yl)-3-(1-(2,2,2-trifluoroethyl)-1H- |
| | indazole-3-carboxamido)benzamido)-5-fluorophenyl)acetic acid |
| 77 | (R)-2-(5-fluoro-2-(4-(2-methylpiperidin-1-yl)-3-(1-(2,2,2-trifluoroethyl)-1H- |
| | indazole-3-carboxamido)benzamido)phenyl)acetic acid |
| 78 | (S)-2-(5-fluoro-2-(4-(2-methylpiperidin-1-yl)-3-(1-(2,2,2-trifluoroethyl)-1H- |
| | indazole-3-carboxamido)benzamido)phenyl)acetic acid |
| 79 | (R)-2-(5-fluoro-2-(4-(3-methylpiperidin-1-yl)-3-(1-(2,2,2-trifluoroethyl)-1H- |
| | indazole-3-carboxamido)benzamido)phenyl)acetic acid |
| 80 | (S)-2-(5-fluoro-2-(4-(3-methylpiperidin-1-yl)-3-(1-(2,2,2-trifluoroethyl)-1H- |
| | indazole-3-carboxamido)benzamido)phenyl)acetic acid |
| 81 | 2-(2-(4-(2-(difluoromethyl)piperidin-1-yl)-3-(1-(2,2,2-trifluoroethyl)-1H- |
| | indazole-3-carboxamido)benzamido)-5-fluorophenyl)acetic acid |
| 82 | 2-(2-(4-(3-(difluoromethyl)piperidin-1-yl)-3-(1-(2,2,2-trifluoroethyl)-1H- |
| | indazole-3-carboxamido)benzamido)-5-fluorophenyl)acetic acid |
| 83 | 2-(2-(4-(4-(difluoromethyl)piperidin-1-yl)-3-(1-(2,2,2-trifluoroethyl)-1H- |
| | indazole-3-carboxamido)benzamido)-5-fluorophenyl)acetic acid |
| 84 | 2-(5-fluoro-2-(4-(3-methoxypiperidin-1-yl)-3-(1-(2,2,2-trifluoroethyl)-1H- |
| | indazole-3-carboxamido)benzamido)phenyl)acetic acid |
| 85 | 2-(5-fluoro-2-(4-(3-hydroxypiperidin-1-yl)-3-(1-(2,2,2-trifluoroethyl)-1H- |
| | indazole-3-carboxamido)benzamido)phenyl)acetic acid |
| 86 | 2-(5-fluoro-2-(4-(3-(hydroxymethyl)piperidin-1-yl)-3-(1-(2,2,2-trifluoroethyl)- |
| | 1H-indazole-3-carboxamido)benzamido)phenyl)acetic acid |
| 87 | 2-(2-(4-(3-cyanopiperidin-1-yl)-3-(1-(2,2,2-trifluoroethyl)-1H-indazole-3- |
| | carboxamido)benzamido)-5-fluorophenyl)acetic acid |
| 88 | 2-(2-(4-(3-carbamoylpiperidin-1-yl)-3-(1-(2,2,2-trifluoroethyl)-1H-indazole-3- |
| | carboxamido)benzamido)-5-fluorophenyl)acetic acid |
| 89 | 2-(2-(4-(3-ethylpiperidin-1-yl)-3-(1-(2,2,2-trifluoroethyl)-1H-indazole-3- |
| | carboxamido)benzamido)-5-fluorophenyl)acetic acid |
| 90 | 2-(2-(4-(4-ethylpiperidin-1-yl)-3-(1-(2,2,2-trifluoroethyl)-1H-indazole-3- |
| | carboxamido)benzamido)-5-fluorophenyl)acetic acid |
| 91 | 2-(2-(4-(4-azaspiro[2.5]octan-4-yl)-3-(1-(2,2,2-trifluoroethyl)-1H-indazole-3- |
| | carboxamido)benzamido)-5-fluorophenyl)acetic acid |
| 92 | 2-(2-(4-(5-azaspiro[2.5]octan-5-yl)-3-(1-(2,2,2-trifluoroethyl)-1H-indazole-3- |
| | carboxamido)benzamido)-5-fluorophenyl)acetic acid |
| 93 | 2-(2-(4-(6-azaspiro[2.5]octan-6-yl)-3-(1-(2,2,2-trifluoroethyl)-1H-indazole-3- |
| | carboxamido)benzamido)-5-fluorophenyl)acetic acid |
| 94 | 2-(2-(3-(1-(cyclopropylmethyl)-1H-indazole-3-carboxamido)-4-(piperidin-1- |
| | yl)benzamido)-5-fluorophenyl)acetic acid |
| 95 | 2-(5-fluoro-2-(3-(1-isobutyl-1H-indazole-3-carboxamido)-4-(piperidin-1- |
| | yl)benzamido)phenyl)acetic acid |
| 96 | 2-(2-(3-(1-(2,2-difluoroethyl)-1H-indazole-3-carboxamido)-4-(piperidin-1- |
| | yl)benzamido)-5-fluorophenyl)acetic acid |
| 97 | 2-(2-(3-(1-cyclopropyl-1H-indazole-3-carboxamido)-4-(piperidin-1- |
| | yl)benzamido)-5-fluorophenyl)acetic acid |
| 98 | 2-(2-(3-(1-(cyclobutylmethyl)-1H-indazole-3-carboxamido)-4-(piperidin-1- |
| | yl)benzamido)-5-fluorophenyl)acetic acid |
| 99 | 2-(5-fluoro-2-(3-(1-(2-hydroxyethyl)-1H-indazole-3-carboxamido)-4-(piperidin- |
| | 1-yl)benzamido)phenyl)acetic acid |
| 100 | 2-(2-(3-(1-(2-aminoethyl)-1H-indazole-3-carboxamido)-4-(piperidin-1- |
| | yl)benzamido)-5-fluorophenyl)acetic acid |
| 101 | 2-(5-fluoro-2-(4-(piperidin-1-yl)-3-(1-(2-(pyrrolidin-1-yl)ethyl)-1H-indazole-3- |
| | carboxamido)benzamido)phenyl)acetic acid |
| 102 | 2-(2-(3-(1-(cyanomethyl)-1H-indazole-3-carboxamido)-4-(piperidin-1- |
| | yl)benzamido)-5-fluorophenyl)acetic acid |
| 103 | 2-(5-fluoro-2-(4-(piperidin-1-yl)-3-(1-(piperidin-4-ylmethyl)-1H-indazole-3- |
| | carboxamido)benzamido)phenyl)acetic acid |
| 104 | 2-(5-fluoro-2-(4-(piperidin-1-yl)-3-(1-((tetrahydro-2H-pyran-4-yl)methyl)-1H- |
| | indazole-3-carboxamido)benzamido)phenyl)acetic acid |
| 105 | 2-(5-fluoro-2-(3-(1-(2-methoxyethyl)-1H-indazole-3-carboxamido)-4-(piperidin- |
| | 1-yl)benzamido)phenyl)acetic acid |
| 106 | 2-(5-fluoro-2-(3-(1-(2-hydroxy-2-methylpropyl)-1H-indazole-3-carboxamido)-4- |
| | (piperidin-1-yl)benzamido)phenyl)acetic acid |
| 107 | 2-(5-fluoro-2-(4-(piperidin-1-yl)-3-(1-(2,2,2-trifluoroethyl)-1H-pyrazolo[4,3- |
| | c]pyridine-3-carboxamido)benzamido)phenyl)acetic acid |
| 108 | 2-(5-fluoro-2-(3-(imidazo[1,2-a]pyridine-3-carboxamido)-4-(piperidin-1- |
| | yl)benzamido)phenyl)acetic acid |
| 109 | 2-(5-fluoro-2-(3-(imidazo[1,2-a]pyridine-2-carboxamido)-4-(piperidin-1- |
| | yl)benzamido)phenyl)acetic acid |
| 110 | 2-(5-fluoro-2-(3-(1-methylimidazo[1,5-a]pyridine-3-carboxamido)-4-(piperidin- |
| | 1-yl)benzamido)phenyl)acetic acid |
| 111 | 2-(5-fluoro-2-(4-(piperidin-1-yl)-3-(1-(2,2,2-trifluoroethyl)-1H-pyrazolo[4,3- |
| | b]pyridine-3-carboxamido)benzamido)phenyl)acetic acid |
| 112 | 2-(5-fluoro-2-(4-(piperidin-1-yl)-3-(1-(2,2,2-trifluoroethyl)-1H-pyrazolo[3,4- |
| | c]pyridine-3-carboxamido)benzamido)phenyl)acetic acid |
| 113 | 2-(5-fluoro-2-(4-(piperidin-1-yl)-3-(1-(2,2,2-trifluoroethyl)-1H-pyrazolo[3,4- |
| | b]pyridine-3-carboxamido)benzamido)phenyl)acetic acid |
| 114 | 2-(5-fluoro-2-(4-(piperidin-1-yl)-3-((1-(2,2,2-trifluoroethyl)-1H-indazole)-3- |
| | sulfonamido)benzamido)phenyl)acetic acid |
| 115 | 2-(5-fluoro-2-(3-(4-phenyl-1-(2,2,2-trifluoroethyl)-1H-pyrazole-3-carboxamido)- |
| | 4-(piperidin-1-yl)benzamido)phenyl)acetic acid |
| 116 | 2-(5-fluoro-2-(3-(5-phenyl-1-(2,2,2-trifluoroethyl)-1H-pyrazole-3-carboxamido)- |
| | 4-(piperidin-1-yl)benzamido)phenyl)acetic acid |
| 117 | 2-(2-(3-(4-cyclopropyl-1-(2,2,2-trifluoroethyl)-1H-pyrazole-3-carboxamido)-4- |
| | (piperidin-1-yl)benzamido)-5-fluorophenyl)acetic acid |
| 118 | 2-(5-fluoro-2-(4-(piperidin-1-yl)-3-(1-((tetrahydrofuran-3-yl)methyl)-1H- |
| | indazole-3-carboxamido)benzamido)phenyl)acetic acid |
| 119 | 2-(5-fluoro-2-(4-(piperidin-1-yl)-3-(1-((tetrahydrofuran-2-yl)methyl)-1H- |
| | indazole-3-carboxamido)benzamido)phenyl)acetic acid |
| 120 | 2-(5-fluoro-2-(3-(1-(2-morpholinoethyl)-1H-indazole-3-carboxamido)-4- |
| | (piperidin-1-yl)benzamido)phenyl)acetic acid |
| 121 | 2-(5-fluoro-2-(4-(piperidin-1-yl)-3-(1-(3,3,3-trifluoropropyl)-1H-indazole-3- |
| | carboxamido)benzamido)phenyl)acetic acid |
| 122 | 2-(5-fluoro-2-(4-(piperidin-1-yl)-3-(1-(prop-2-yn-1-yl)-1H-indazole-3- |
| | carboxamido)benzamido)phenyl)acetic acid |
| 123 | 2-(2-(3-(1-(but-2-yn-1-yl)-1H-indazole-3-carboxamido)-4-(piperidin-1- |
| | yl)benzamido)-5-fluorophenyl)acetic acid |
| 124 | 2-(5-fluoro-2-(3-(1-((5-methylisoxazol-3-yl)methyl)-1H-indazole-3- |
| | carboxamido)-4-(piperidin-1-yl)benzamido)phenyl)acetic acid |
| 125 | 2-(5-fluoro-2-(3-(1-((5-methyl-1,2,4-oxadiazol-3-yl)methyl)-1H-indazole-3- |
| | carboxamido)-4-(piperidin-1-yl)benzamido)phenyl)acetic acid |
| 126 | 2-(5-fluoro-2-(3-(1-((5-methyl-1,3,4-oxadiazol-2-yl)methyl)-1H-indazole-3- |
| | carboxamido)-4-(piperidin-1-yl)benzamido)phenyl)acetic acid |
| 127 | 2-(2-(3-(1-((1-cyanocyclopropyl)methyl)-1H-indazole-3-carboxamido)-4- |
| | (piperidin-1-yl)benzamido)-5-fluorophenyl)acetic acid |
| 128 | 2-(5-fluoro-2-(4-(piperidin-1-yl)-3-(1-(2,2,2-trifluoroethyl)-1H-indole-3- |
| | carboxamido)benzamido)phenyl)acetic acid |
| 129 | 2-(5-fluoro-2-(4-(piperidin-1-yl)-3-(3-(1-(2,2,2-trifluoroethyl)-1H-indazol-3- |
| | yl)ureido)benzamido)phenyl)acetic acid |
| 130 | 2-(5-fluoro-2-(3-(2-methyl-1-(2,2,2-trifluoroethyl)-1H-indole-3-carboxamido)-4- |
| | (piperidin-1-yl)benzamido)phenyl)acetic acid |
| 131 | 2-(5-fluoro-2-(4-(piperidin-1-yl)-3-(1-(2-(trifluoromethoxy)ethyl)-1H-indazole- |
| | 3-carboxamido)benzamido)phenyl)acetic acid |
| 132 | 2-(2-(3-(1-((1,4-dioxan-2-yl)methyl)-1H-indazole-3-carboxamido)-4-(piperidin- |
| | 1-yl)benzamido)-5-fluorophenyl)acetic acid |
| 133 | 2-(5-fluoro-2-(4-(piperidin-1-yl)-3-(1-((tetrahydro-2H-pyran-3-yl)methyl)-1H- |
| | indazole-3-carboxamido)benzamido)phenyl)acetic acid |
| 134 | 2-(5-fluoro-2-(3-(3-phenyl-1-(2,2,2-trifluoroethyl)-1H-pyrazole-5-carboxamido)- |
| | 4-(piperidin-1-yl)benzamido)phenyl)acetic acid |
| 135 | 2-(5-fluoro-2-(4-(piperidin-1-yl)-3-(2-(2,2,2-trifluoroethyl)-2H-indazole-3- |
| | carboxamido)benzamido)phenyl)acetic acid hydrochloride |
| 136 | (S)-2-(5-fluoro-2-(4-(piperidin-1-yl)-3-(1-((tetrahydrofuran-2-yl)methyl)-1H- |
| | indazole-3-carboxamido)benzamido)phenyl)acetic acid |
| 137 | (R)-2-(5-fluoro-2-(4-(piperidin-1-yl)-3-(1-((tetrahydrofuran-2-yl)methyl)-1H- |
| | indazole-3-carboxamido)benzamido)phenyl)acetic acid |
| 138 | 2-(5-fluoro-2-(3-(5-fluoro-1-(2,2,2-trifluoroethyl)-1H-indazole-3-carboxamido)- |
| | 4-(piperidin-1-yl)benzamido)phenyl)acetic acid |
| 139 | 2-(5-chloro-2-(3-(5-fluoro-1-(2,2,2-trifluoroethyl)-1H-indazole-3-carboxamido)- |
| | 4-(piperidin-1-yl)benzamido)phenyl)acetic acid |
| 140 | 2-(5-fluoro-2-(3-(5-methoxy-1-(2,2,2-trifluoroethyl)-1H-indazole-3- |
| | carboxamido)-4-(piperidin-1-yl)benzamido)phenyl)acetic acid |
| 141 | 2-(5-fluoro-2-(3-(4-methyl-1-(2,2,2-trifluoroethyl)-1H-indazole-3-carboxamido)- |
| | 4-(piperidin-1-yl)benzamido)phenyl)acetic acid |
| 142 | 2-(5-fluoro-2-(3-(5-methyl-1-(2,2,2-trifluoroethyl)-1H-indazole-3-carboxamido)- |
| | 4-(piperidin-1-yl)benzamido)phenyl)acetic acid |
| 143 | 2-(5-fluoro-2-(3-(6-methyl-1-(2,2,2-trifluoroethyl)-1H-indazole-3-carboxamido)- |
| | 4-(piperidin-1-yl)benzamido)phenyl)acetic acid |
| 144 | 2-(5-fluoro-2-(3-(7-methyl-1-(2,2,2-trifluoroethyl)-1H-indazole-3-carboxamido)- |
| | 4-(piperidin-1-yl)benzamido)phenyl)acetic acid |
| 145 | 2-(5-fluoro-2-(4-(piperidin-1-yl)-3-(1-(2,2,2-trifluoroethyl)-1,4,5,6- |
| | tetrahydrocyclopenta[c]pyrazole-3-carboxamido)benzamido)phenyl)acetic acid |
| 146 | 2-(5-fluoro-2-(4-(piperidin-1-yl)-3-(1-(1,1,1-trifluoropropan-2-yl)-1H-indazole- |
| | 3-carboxamido)benzamido)phenyl)acetic acid |
| 147 | 2-(2-(3-(1-(1-cyclopropylethyl)-1H-indazole-3-carboxamido)-4-(piperidin-1- |
| | yl)benzamido)-5-fluorophenyl)acetic acid |
| 148 | 2-(5-fluoro-2-(4-(piperidin-1-yl)-3-(1-(1-(tetrahydro-2H-pyran-4-yl)ethyl)-1H- |
| | indazole-3-carboxamido)benzamido)phenyl)acetic acid |
| 149 | 2-(2-(3-((1-(cyanomethyl)-1H-indazol-3-yl)carbamoyl)-4-(piperidin-1- |
| | yl)benzamido)-5-fluorophenyl)acetic acid |
| 150 | 2-(5-fluoro-2-(4-(piperidin-1-yl)-3-((1-((tetrahydro-2H-pyran-4-yl)methyl)-1H- |
| | indazol-3-yl)carbamoyl)benzamido)phenyl)acetic acid |
| 151 | 2-(5-fluoro-2-(4-(piperidin-1-yl)-3-((1-(2,2,2-trifluoroethyl)-4,5,6,7-tetrahydro- |
| | 1H-indazol-3-yl)carbamoyl)benzamido)phenyl)acetic acid |
| 152 | (R)-2-(2-(4-(2-ethylpiperidin-1-yl)-3-(1-(2,2,2-trifluoroethyl)-1H-indazole-3- |
| | carboxamido)benzamido)-5-fluorophenyl)acetic acid |
| 153 | (S)-2-(2-(4-(2-ethylpiperidin-1-yl)-3-(1-(2,2,2-trifluoroethyl)-1H-indazole-3- |
| | carboxamido)benzamido)-5-fluorophenyl)acetic acid |
| 154 | 2-(5-fluoro-2-(4-(piperidin-1-yl)-3-(1-(2,2,2-trifluoroethyl)-4,5,6,7-tetrahydro- |
| | 1H-indazole-3-carboxamido)benzamido)phenyl)acetic acid |
| 155 | 2-(2-(3-(5,5-difluoro-5,6-dihydro-4H-pyrrolo[1,2-b]pyrazole-2-carboxamido)-4- |
| | (piperidin-1-yl)benzamido)-5-fluorophenyl)acetic acid |
| 156 | 2-(5-fluoro-2-(4-(piperidin-1-yl)-3-(1-(2,2,2-trifluoroethyl)-1,4,6,7- |
| | tetrahydropyrano[4,3-c]pyrazole-3-carboxamido)benzamido)phenyl)acetic acid |
| 157 | 2-(5-fluoro-2-(4-(piperidin-1-yl)-3-(2-(1-(2,2,2-trifluoroethyl)-1H-indazol-3- |
| | yl)acetamido)benzamido)phenyl)acetic acid |
| 158 | 2-(5-fluoro-2-(4-(piperidin-1-yl)-3-(1-(2,2,2-trifluoroethyl)-1H-indazole-3- |
| | carboxamido)benzamido)phenyl)acetic acid; |
| 159 | 2-(2-(3-(1-benzyl-1H-indazole-3-carboxamido)-4-(piperidin-1-yl)benzamido)-5- |
| | fluorophenyl)acetic acid; |
| 160 | 2-(2-(3-(1-ethyl-1H-indazole-3-carboxamido)-4-(piperidin-1-yl)benzamido)-5- |
| | fluorophenyl)acetic acid; |
| 161 | 2-(2-(4-(piperidin-1-yl)-3-(4,5,6,7-tetrahydropyrazolo[1,5-a]pyridine-2- |
| | carboxamido)benzamido)phenyl)acetic acid; |
| 162 | 2-(5-fluoro-2-(3-(1-isopropyl-1H-indazole-3-carboxamido)-4-(piperidin-1 - |
| | yl)benzamido)phenyl)acetic acid; |
| 163 | 2-(2-(3-(1-methyl-5-phenyl-1H-pyrazole-3-carboxamido)-4-(piperidin-1- |
| | yl)benzamido)phenyl)acetic acid; |
| 164 | N-(5-((4-(1H-tetrazol-5-yl)phenyl)carbamoyl)-2-(piperidin-1-yl)phenyl)-1-(2,2,2- |
| | trifluoroethyl)-1H-indazole-3-carboxamide; |
| 165 | N-(5-((4-(1H-tetrazol-5-yl)phenyl)carbamoyl)-2-(piperidin-1-yl)phenyl)-6-fluoro-1- |
| | methyl-1H-indazole-3-carboxamide; |
| 166 | N-(5-((2-fluoro-5-(1H-tetrazol-5-yl)phenyl)carbamoyl)-2-(piperidin-1-yl)phenyl)-1- |
| | methyl-1H-indazole-3-carboxamide; |
| 167 | 5-(6-(1H-tetrazol-5-yl)-2H-indazol-2-yl)-2-(piperidin-1-yl)-N-(p-tolyl)benzamide; |
| 168 | N-(4-methylphenyl)-5-({[4-(5-oxo-4,5-dihydro-1,2,4-thiadiazol-3- |
| | yl)phenyl]methyl}amino)-2-(piperidin-1-yl)benzamide; |
| 169 | 5-((2-fluoro-5-(1H-tetrazol-5-yl)benzyl)amino)-2-(piperidin-1-yl)-N-(p- |
| | tolyl)benzamide; |
| 170 | 5-(1-oxo-5-(1H-tetrazol-5-yl)isoindolin-2-yl)-2-(piperidin-1-yl)-N-(p-tolyl)benzamide; |
| | and |
| 172 | 1-ethyl-N-(5-((2-(2-(methylsulfonamido)-2-oxoethyl)phenyl)carbamoyl)-2-(piperidin-1- |
| | yl)phenyl)-1H-indazole-3-carboxamide; |

or a pharmaceutically acceptable salt, isomer, solvate, prodrug or tautomer thereof.

38. The compound of any one of the previous claims, wherein the compound is selected from:

| Compound No. | Compound Name |
|---|---|
| 7 | 2-(5-fluoro-2-(4-(piperidin-1-yl)-3-(1-(2,2,2-trifluoroethyl)-1H-indazole-3- |
| | carboxamido)benzamido)phenyl)acetic acid |
| 30 | 2-(5-fluoro-2-(4-(piperidin-1-yl)-3-(1-(2,2,2-trifluoroethyl)-1H-indazole-3- |
| | carboxamido)benzamido)phenyl)-2-methylpropanoic acid |
| 31 | 1-(5-fluoro-2-(4-(piperidin-1-yl)-3-(1-(2,2,2-trifluoroethyl)-1H-indazole-3- |
| | carboxamido)benzamido)phenyl)cyclopropane-1-carboxylic acid |
| 73 | 2-(5-fluoro-2-(4-(3-methylpiperidin-1-yl)-3-(1-(2,2,2-trifluoroethyl)-1H- |
| | indazole-3-carboxamido)benzamido)phenyl)acetic acid |
| 122 | 2-(5-fluoro-2-(4-(piperidin-1-yl)-3-(1-(prop-2-yn-1-yl)-1H-indazole-3- |
| | carboxamido)benzamido)phenyl)acetic acid |
| 22 | 2-(2-(4-(piperidin-1-yl)-3-(1-(2,2,2-trifluoroethyl)-1H-indazole-3- |
| | carboxamido)benzamido)-5-(trifluoromethyl)phenyl)acetic acid |
| 28 | 2-(5-fluoro-2-(4-(piperidin-1-yl)-3-(1-(2,2,2-trifluoroethyl)-1H-indazole-3- |
| | carboxamido)benzamido)phenyl)butanoic acid |
| 61 | 2-(2-(4-(4,4-dimethylpiperidin-1-yl)-3-(1-(2,2,2-trifluoroethyl)-1H-indazole- |
| | 3-carboxamido)benzamido)-5-fluorophenyl)acetic acid |
| 104 | 2-(5-fluoro-2-(4-(piperidin-1-yl)-3-(1-((tetrahydro-2H-pyran-4-yl)methyl)-1H- |
| | indazole-3-carboxamido)benzamido)phenyl)acetic acid |
| 121 | 2-(5-fluoro-2-(4-(piperidin-1-yl)-3-(1-(3,3,3-trifluoropropyl)-1H-indazole-3- |
| | carboxamido)benzamido)phenyl)acetic acid |
| 40 | 2-(2-(4-(4,4-difluoropiperidin-1-yl)-3-(1-(2,2,2-trifluoroethyl)-1H-indazole-3- |
| | carboxamido)benzamido)-5-fluorophenyl)acetic acid |
| 25 | 2-(3-chloro-2-(4-(piperidin-1-yl)-3-(1-(2,2,2-trifluoroethyl)-1H-indazole-3- |
| | carboxamido)benzamido)phenyl)acetic acid |
| 123 | 2-(2-(3-(1-(but-2-yn-1-yl)-1H-indazole-3-carboxamido)-4-(piperidin-1- |
| | yl)benzamido)-5-fluorophenyl)acetic acid |
| 158 | 2-(5-fluoro-2-(4-(piperidin-1-yl)-3-(1-(2,2,2-trifluoroethyl)-1H-indazole-3- |
| | carboxamido)benzamido)phenyl)acetic acid; |
| 159 | 2-(2-(3-(1-benzyl-1H-indazole-3-carboxamido)-4-(piperidin-1-yl)benzamido)-5- |
| | fluorophenyl)acetic acid; |
| 160 | 2-(2-(3-(1-ethyl-1H-indazole-3-carboxamido)-4-(piperidin-1-yl)benzamido)-5- |
| | fluorophenyl)acetic acid; |
| 161 | 2-(2-(4-(piperidin-1-yl)-3-(4,5,6,7-tetrahydropyrazolo[1,5-a]pyridine-2- |
| | carboxamido)benzamido)phenyl)acetic acid; |
| 162 | 2-(5-fluoro-2-(3-(1-isopropyl-1H-indazole-3-carboxamido)-4-(piperidin-1- |
| | yl)benzamido)phenyl)acetic acid; and |
| 163 | 2-(2-(3-(1-methyl-5-phenyl-1H-pyrazole-3-carboxamido)-4-(piperidin-1- |
| | yl)benzamido)phenyl)acetic acid |

or a pharmaceutically acceptable salt, isomer, solvate, prodrug, or tautomer thereof.

39. A pharmaceutical composition comprising the compound and pharmaceutically acceptable salts, prodrugs, solvates, hydrates, isomers, or tautomers thereof of any one of claims 1-38, and a pharmaceutically acceptable carrier.

40. The pharmaceutical composition of claim 39, further comprising an additional pharmaceutically active agent.

41. A method of activating an LPL enzyme in a cell, the method comprising contacting the cell with the compound and pharmaceutically acceptable salts, prodrugs, solvates, hydrates, isomers, or tautomers thereof of any one of claims 1-38.

42. The method of claim 41, wherein the contacting is in vitro or in vivo.

43. The method of claim 41 or 42, wherein the subject is a mammal.

44. The method of claim 43, wherein the subject is a human.

45. A method for the treatment or prevention of a condition comprising administering to a subject in need thereof a therapeutically effective amount of a composition comprising the compound of any one of claims 1-38, or combination thereof.

46. The method of claim 45, wherein the condition is selected from hypertriglyceridemia, moderate hypertriglyceridemia, severe hypertriglyceridemia, mixed dyslipidemia, familial chylomicronemia, type 1 hyperlipoproteinemia, type Ib hyperlipoproteinemia, type III hyperlipoproteinemia, type IV hyperlipoproteinemia, type V hyperlipoproteinemia, metabolic syndrome, partial lipodystrophy, prediabetes, type 11 diabetes, atherosclerotic cardiovascular disease, myocardial infarction, coronary heart disease, ischemic heart disease, peripheral vascular disease, cerebrovascular disease, peripheral arterial disease, acute pancreatitis.

47. The method of claim 46, wherein the subject has high polygenic risk for coronary heart disease.

48. The method of claim 46, wherein the subject has high polygenic risk for hypertriglyceridemia.

49. The method of claim 46, wherein the subject has high polygenic risk for lipodystrophy.

50. The method of claim 46, wherein the subject has high polygenic risk for peripheral vascular disease.

51. The method of claim 46, wherein the subject has high polygenic risk for cerebrovascular disease.

52. The method of claim 46, wherein the subject has rare damaging heterozygous or homozygous mutations in LPL, APOC2, APOAS, LMF1, GPIHBPI, APOE, APOB.
